(19) United States
(12) Patent Application Publication
(10) Pub. No.: US 2020/0308660 A1
(43) Pub. Date: Oct. 1, 2020

(54) QUANTITATIVE TRAIT LOCI AFFECTING FATTY ACID COMPOSITION IN MILK

(71) Applicants: TINE SA, Oslo (NO); GENO SA, Hamar (NO)

(72) Inventors: Sigbjørn Lien, SON (NO); Hanne Gro Olsen, ÅS (NO); Tim Martin Knutsen, ÅS (NO); Harald Martens, Trondheim (NO); Achim Kohler, ÅS (NO)

(21) Appl. No.: 16/312,654

(22) PCT Filed: Jun. 27, 2017
(86) PCT No.: PCT/EP2017/065798

§ 371 Date: Dec. 21, 2018

(30) Foreign Application Priority Data
Jun. 29, 2016 (NO) 20161081
Jan. 31, 2017 (NO) 20170149

(51) Int. Cl. C12Q 1/6888

(52) U.S. Cl. CPC C12Q 1/6888; C12Q 2600/124; C12Q 2600/156

(57) ABSTRACT

The present invention relates to polymorphisms indicative of altered milk fatty acid composition in female milk-producing cattle. In particular, the present invention provides methods for selecting a cattle which possesses a genotype which in female milk-producing cattle is indicative of a desired milk fatty acid composition and cattle selected by said method. Further, the present invention provides milk produced by the female milk-producing cattle, methods for selective breeding and non-human gametes. Use of a nucleic acid molecule or an oligonucleotide in an in vitro method for determining the presence of at least one allele, which in a female milk-producing cattle is indicative of a desired milk fatty acid composition, is also part of the present invention.

### FIELD OF THE INVENTION

The present invention relates to polymorphisms indicative of altered milk fatty acid composition in female milk-producing cattle. In particular, the present invention provides methods for selecting a cattle which possesses a genotype which in female milk-producing cattle is indicative of a desired milk fatty acid composition and cattle selected by said method. Further, the present invention provides milk produced by the female milk-producing cattle, methods for selective breeding and non-human gametes. Use of a nucleic acid molecule or an oligonucleotide in an in vitro method for determining the presence of at least one allele, which in a female milk-producing cattle is indicative of a desired milk fatty acid composition, is also part of the present invention.

### BACKGROUND OF THE INVENTION

Bovine milk is widely regarded as a valuable food source in human nutrition, and serves as an important source of proteins, minerals, vitamins and fats in western diets. In addition to being an important source of energy, the milk fat contains valuable fat-soluble vitamins and bio-active lipid components.

Of the roughly 400 different fatty acids found in Bovine milk, only around 15 are present at the 1% level or higher. The milk fatty acids are derived via two major pathways: direct transport from rumen to mammary gland by the blood, and de novo synthesis in the mammary gland. The short- and medium-chained saturated fatty acids C4:0 to C14:0 together with about half of the C16:0 are synthesized de novo in the mammary gland from acetate and β-hydroxybutyrate. Acetate and butyric acid are generated in the rumen by fermentation of feed components and butyric acid is converted to β-hydroxybutyrate during absorption through the rumen epithelium. The remaining C16:0 and the long-chain fatty acids typically originate from dietary lipids and from lipolysis of adipose tissue triacylglycerols.

Both the long- and medium-chained fatty acids may be desaturated by Δ⁹-desaturase to their cis-9 monounsaturated counterparts. Monounsaturated fatty acids constitutes approximately 25% of the fatty acids in milk, with oleic acid (18:1) accounting for about 24% by weight of the total fatty acids. Poly-unsaturated fatty acids constitutes about 2% by weight of the total fatty acids and the main poly-unsaturated fatty acids are linoleic acid (18:2) and α-linolenic acid (18:3) accounting for 1.6 and 0.7% by weight of the total fatty acids.

Due to ruminal biohydrogenation of unsaturated fatty acids from the diet, the saturated fatty acids present in milk account for approximately 70% by weight of the total fatty acids. The most important saturated fatty acid from a quantitative viewpoint is palmitic acid (16:0), which accounts for approximately 30% by weight. Myristic acid (14:0) and stearic acid (18:0) make up 11 and 12% by weight, respectively. Of the saturated fatty acids, about 10.9% are short-chain fatty acids (C4:0-C10:0).

The net effect of dairy fat on human health is debated because while mono- and polyunsaturated fatty acids as well as short saturated fatty acids typically have been associated with positive effects on cardiovascular health and diabetes, medium and long-chain saturated fatty acids have been associated with cardiovascular disease and obesity. It is therefore of great interest to identify factors that may influence fatty acid composition in bovine milk.

Many factors are known to be associated with variations in the amount and fatty acid composition of bovine milk lipids. They may be of animal origin, i.e. related to genetics (breeding and selection), stage of lactation, mastitis and ruminal fermentation, or they may be feed-related factors, i.e. related to fiber and energy intake, dietary fats, and seasonal and regional effects.

The present inventors have identified factors of animal origin, i.e. related to genetics that influence the fatty acid composition of bovine milk. The factors identified are polymorphisms, including single nucleotide polymorphisms (SNP), within the bovine genome which in a female milk-producing cattle influence the milk fatty acid composition.

### SUMMARY OF THE INVENTION

Method for Selecting a Cattle

The present invention provides in a first aspect a method for selecting a cattle which possesses a genotype which in a female milk-producing cattle is indicative of a desired milk fatty acid composition, the method comprising:

determining the presence of at least one allele, which in a female milk-producing cattle is indicative of a desired milk fatty acid composition, within the genome of said cattle; and

selecting said cattle when the at least one allele, which in a female milk-producing cattle is indicative of a desired milk fatty acid composition, is present.

According to particular embodiments, the at least one allele is an allele of at least one polymorphism selected from the polymorphisms listed in table 1. The at least one allele is preferably an allele of at least one polymorphism selected from the group consisting of P #1 to P #916, such as P #1 to P #310.

According to other particular embodiments, the at least one allele is an allele of at least one polymorphism selected from the group consisting of P #1 to P #916, such as P #1 to P #310.

According to other particular embodiments, a desired milk fatty acid composition is decreased amount of C16:0 in milk; increased amount of C18:1 in milk; increased amount of C14:1 cis-9 in milk; increased amount of one or more fatty acids selected from the group consisting of C6:0, C8:0, C10:0, C12:0 and C14:0 in milk; or any combination thereof.

According to other particular embodiments, the at least one allele is a non-fat allele” for C16:0 of at least one polymorphism selected from the polymorphisms listed in table 1; “fat allele” for C18:1 of at least one polymorphism selected from the polymorphisms listed in table 1; “fat allele” for C14:1 cis-9 of at least one polymorphism selected from the polymorphisms listed in table 1; “fat allele” for C6:0 of at least one polymorphism selected from the polymorphisms listed in table 1; “fat allele” for C8:0 of at least one polymorphism selected from the polymorphisms listed in table 1; “fat allele” for C10:0 of at least one polymorphism selected from the polymorphisms listed in table 1; “fat allele” for C12:0 of at least one polymorphism selected from the polymorphisms listed in table 1; “fat allele” for C14:0 of at least one polymorphism selected from the polymorphisms listed in table 1; or any combination thereof.

According to other particular embodiments, the at least one allele is:

a “non-fat allele” for C16:0 of at least one polymorphism selected from the group consisting of P #33, P #241-P #248, P #303-P #312, P #314-P #344, P #346-P #475 and, P #477-P #481;

a “fat allele” for C18:1 of at least one polymorphism selected from the group consisting of P #19, P #34, P #38, P #39, P #141, P #148, P #153, P #233-P #240, P #242-P #246, P #271-P #278, P #280-P #283, P #285, P #289-P #302, P #311-P #334, P #339, P #340, P #343-P #475, P #477-P #481, P #484-P #494, P #497, P #543, P #588, P #589, P #916, P #911, P #14, P #37, P #903 and P #7;

a “fat allele” for C14:1 cis-9 of at least one polymorphism selected from the group consisting of P #243-P #246, P #314-P #332, P #334-P #340, P #346-P #352, P #354-P #363, P #366-P #392, P #394-P #396, P #398-P #410, P #412-P #475 and P #477-P #481;

a “fat allele” for C6:0 of at least one polymorphism selected from the group consisting of P #1, P #12, P #15-P #17, P #24, P #41, P #63, P #64, P #69-P #71, P #74-P #79, P #83, P #85-P #87, P #89-P #93, P #97-P #103, P #112-P #118, P #120, P #121, P #123-P #135, P #138-P #141, P #148, P #153-P #168, P #218, P #220-P #226, P #249, P #250, P #253-P #260, P #267, P #495, P #499, P #501, P #503-P #514, P #517-P #539, P #541, P #542, P #544-P #547, P #554-P #585, P #590-P #592, P #595-P #597, P #599-P #614, P #617-P #621, P #623-P #652, P #654-P #669, P #672-P #674, P #676-P #678, P #681, P #682, P #688-P #744, P #746-P #769, P #771-P #813, P #815, P #818-P #824, P #826-P #831 and P #872;

a “fat allele” for C8:0 of at least one polymorphism selected from the group consisting of P #1, P #10-P #12, P #16-P #19, P #40-P #45, P #48, P #49, P #51, P #56-P #60, P #63-P #66, P #68-P #93, P #97, P #99-P #101, P #103-P #108, P #111-P #118, P #120, P #121, P #123-P #218, P #220, P #224-P #231, P #254, P #258, P #259, P #268, P #270, P #285-P #288, P #302, P #495, P #496, P #498-P #539, P #541-P #551, P #554-P #586, P #590-P #594, P #628, P #682 and P #683;

a “fat allele” for C10:0 of at least one polymorphism selected from the group consisting of P #3, P #8-P #10, P #16-P #20, P #49-P #51, P #56-P #62, P #64, P #69-P #72, P #74, P #76-P #78, P #80-P #96, P #104-P #109, P #111-P #115, P #117, P #118, P #120, P #121, P #127, P #128, P #132, P #133, P #136-P #142, P #148-P #153, P #156-P #166, P #224-P #226, P #285-P #288, P #297-P #302, P #490-P #494, P #501-P #514, P #517-P #539, P #541-P #547, P #551, P #554-P #580, P #585, P #586, P #589-P #593, P #628, P #889-P #891, P #899, P #900, P #902-P #916;

a “fat allele” for C12:0 of at least one polymorphism selected from the group consisting of P #3, P #9, P #10, P #16-P #20, P #49-P #53, P #61, P #62, P #64, P #67, P #69-P #72, P #74, P #83, P #85-P #87, P #89-P #96, P #104-P #113, P #115, P #117-P #122, P #127, P #128, P #132, P #133, P #138, P #139, P #141, P #142, P #148-P #151, P #153, P #219, P #224-P #226, P #276, P #285-P #288, P #290-P #302, P #487-P #495, P #497, P #501-P #515, P #517-P #547, P #551, P #554-P #580, P #585, P #586, P #588-P #594, P #887-P #891, P #893-P #916;

a “fat allele” for C14:0 of at least one polymorphism selected from the group consisting of P #9, P #10, P #17-P #19, P #34, P #69, P #70, P #83, P #85, P #117, P #120, P #121, P #127, P #128, P #132, P #133, P #139, P #141, P #148-P #151, P #153, P #224-P #226, P #275, P #276, P #279-P #281, P #284-P #302, P #483-P #494, P #497, P #501-P #514, P #517-P #539, P #541, P #543-P #580, P #584-P #594, P #889-P #892, P #899, P #900, P #902-P #916; or

any combination thereof.

According to other particular embodiments, the at least one allele is a “non-fat allele” for C16:0 of at least one polymorphism selected from the group consisting of P #33, P #241-P #248, P #303-P #312, P #314-P #344, P #346-P #475 and, P #477-P #481.

The presence of a “fat-allele” for a specific trait is indicative of increased amount of that trait in milk; and the presence of a “non-fat allele” for a specific trait is indicative of decreased amount of that trait in milk. “fat allele”, “non-fat allele” and the respective traits are specified in table 1.

According to other particular embodiments, the present invention provides a method for selecting a cattle which possesses a genotype which in a female milk-producing cattle is indicative of a desired milk fatty acid composition, the method comprising:

determining the identity of one or more nucleotides of at least one allele of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 1 to 916, such as SEQ ID NOs: 1 to 310; or at a position corresponding to position 60 of a nucleotide sequence which is derived from any one of SEQ ID NOs: 1 to 916, such as SEQ ID NOs: 1 to 310, by 1 to 30 nucleotide substitutions; and

selecting said cattle when the one or more nucleotides of the at least one allele is one or more nucleotides corresponding to the allele which in a female milk-producing cattle is indicative of the desired milk fatty acid composition.

Other particular embodiments relates to a method for selecting a cattle which possesses a genotype which in a female milk-producing cattle is indicative of a desired milk fatty acid composition, the method comprising:

determining the identity of one or more nucleotides of at least one allele of at least one polymorphism, which in a female milk-producing cattle is indicative of decreased amount of C16:0 in milk, within the genome of said cattle, said at least one polymorphism being located within said genome at a position corresponding to position 60 of the nucleotide sequence set forth in any one of SEQ ID NO 33, 241-248, 303-312, 314-344, 346-475 and 477-481; or at a position corresponding to position 60 of a nucleotide sequence which is derived from any one of SEQ ID NO 33, 241-248, 303-312, 314-344, 346-475 and 477-481 by 1 to 30 nucleotide substitutions; and

selecting said cattle when the one or more nucleotides of the at least one allele is one or more nucleotides corresponding to the “non-fat allele” for C16:0.

Non-Human Gamete

The present invention provides in a second aspect a non-human gamete, such as an isolated non-human gamete, comprising within its genome at least one allele which in a female milk-producing cattle is indicative of a desired milk fatty acid composition.

According to particular embodiments, the at least one allele is an allele of at least one polymorphism. The at least one polymorphism may be selected from the polymorphisms listed in table 1.

According to other particular embodiments, the at least one allele is:

a “non-fat allele” for C16:0 of at least one polymorphism selected from the group consisting of P #33, P #241-P #248, P #303-P #312, P #314-P #344, P #346-P #475 and, P #477-P #481;

a “fat allele” for C18:1 of at least one polymorphism selected from the group consisting of P #19, P #34, P #38, P #39, P #141, P #148, P #153, P #233-P #240, P #242-P #246, P #271-P #278, P #280-P #283, P #285, P #289-P #302, P #311-P #334, P #339, P #340, P #343-P #475, P #477-P #481, P #484-P #494, P #497, P #543, P #588, P #589, P #916, P #911, P #14, P #37, P #903 and P #7;

a “fat allele” for C14:1 cis-9 of at least one polymorphism selected from the group consisting of P #243-P #246, P #314-P #332, P #334-P #340, P #346-P #352, P #354-P #363, P #366-P #392, P #394-P #396, P #398-P #410, P #412-P #475 and P #477-P #481;

a “fat allele” for C6:0 of at least one polymorphism selected from the group consisting of P #1, P #12, P #15-P #17, P #24, P #41, P #63, P #64, P #69-P #71, P #74-P #79, P #83, P #85-P #87, P #89-P #93, P #97-P #103, P #112-P #118, P #120, P #121, P #123-P #135, P #138-P #141, P #148, P #153-P #168, P #218, P #220-P #226, P #249, P #250, P #253-P #260, P #267, P #495, P #499, P #501, P #503-P #514, P #517-P #539, P #541, P #542, P #544-P #547, P #554-P #585, P #590-P #592, P #595-P #597, P #599-P #614, P #617-P #621, P #623-P #652, P #654-P #669, P #672-P #674, P #676-P #678, P #681, P #682, P #688-P #744, P #746-P #769, P #771-P #813, P #815, P #818-P #824, P #826-P #831 and P #872;

a “fat allele” for C8:0 of at least one polymorphism selected from the group consisting of P #1, P #10-P #12, P #16-P #19, P #40-P #45, P #48, P #49, P #51, P #56-P #60, P #63-P #66, P #68-P #93, P #97, P #99-P #101, P #103-P #108, P #111-P #118, P #120, P #121, P #123-P #218, P #220, P #224-P #231, P #254, P #258, P #259, P #268, P #270, P #285-P #288, P #302, P #495, P #496, P #498-P #539, P #541-P #551, P #554-P #586, P #590-P #594, P #628, P #682 and P #683;

a “fat allele” for C10:0 of at least one polymorphism selected from the group consisting of P #3, P #8-P #10, P #16-P #20, P #49-P #51, P #56-P #62, P #64, P #69-P #72, P #74, P #76-P #78, P #80-P #96, P #104-P #109, P #111-P #115, P #117, P #118, P #120, P #121, P #127, P #128, P #132, P #133, P #136-P #142, P #148-P #153, P #156-P #166, P #224-P #226, P #285-P #288, P #297-P #302, P #490-P #494, P #501-P #514, P #517-P #539, P #541-P #547, P #551, P #554-P #580, P #585, P #586, P #589-P #593, P #628, P #889-P #891, P #899, P #900, P #902-P #916;

a “fat allele” for C12:0 of at least one polymorphism selected from the group consisting of P #3, P #9, P #10, P #16-P #20, P #49-P #53, P #61, P #62, P #64, P #67, P #69-P #72, P #74, P #83, P #85-P #87, P #89-P #96, P #104-P #113, P #115, P #117-P #122, P #127, P #128, P #132, P #133, P #138, P #139, P #141, P #142, P #148-P #151, P #153, P #219, P #224-P #226, P #276, P #285-P #288, P #290-P #302, P #487-P #495, P #497, P #501-P #515, P #517-P #547, P #551, P #554-P #580, P #585, P #586, P #588-P #594, P #887-P #891, P #893-P #916;

a “fat allele” for C14:0 of at least one polymorphism selected from the group consisting of P #9, P #10, P #17-P #19, P #34, P #69, P #70, P #83, P #85, P #117, P #120, P #121, P #127, P #128, P #132, P #133, P #139, P #141, P #148-P #151, P #153, P #224-P #226, P #275, P #276, P #279-P #281, P #284-P #302, P #483-P #494, P #497, P #501-P #514, P #517-P #539, P #541, P #543-P #580, P #584-P #594, P #889-P #892, P #899, P #900, P #902-P #916; or any combination thereof.

According to other particular embodiments, the at least one allele is a “non-fat allele” for C16:0 of at least one polymorphism selected from the group consisting of P #33, P #241-P #248, P #303-P #312, P #314-P #344, P #346-P #475 and, P #477-P #481.

According to other particular embodiments, the gamete comprises within its genome at least one nucleotide sequence selected from the group consisting of a) the nucleotide sequences set forth in any one of SEQ ID NO:33, 241-248, 303-312, 314-344, 346-475 and 477-481; and b) nucleotide sequences which are derived from any one of SEQ ID NO:33, 241-248, 303-312, 314-344, 346-475 and 477-481 by 1 to 30 nucleotide substitutions;

wherein the one or more nucleotides at position 60 of the nucleotide sequence set forth in any one of SEQ ID NO:33, 241-248, 303-312, 314-344, 346-475 and 477-481 corresponds to the “non-fat allele” for C16:0.

According to other particular embodiments, said non-human gamete is non-human semen or non-human sperm.

According to other particular embodiments, said non-human gamete is non-human ovum.

Method for Selective Breeding

The present invention provides in a third aspect a method for selective breeding of a cattle, the method comprises:

providing non-human semen or non-human sperm comprising within its genome at least one allele which in a female milk-producing cattle is indicative of a desired milk fatty acid composition; and

fertilizing a female (milk-producing) cattle using the non-human semen or non-human sperm.

According to particular embodiments, the present invention provides a method for determining the presence of at least one allele which in a female milk-producing cattle is indicative of a desired milk fatty acid composition within the genome of a female (milk-producing) cattle;

selecting the female (milk-producing) cattle when the at least one allele, which in a female milk-producing cattle is indicative of a desired milk fatty acid composition, is present;

providing non-human semen or non-human sperm comprising within its genome at least one allele which in a female milk-producing cattle is indicative of a desired milk fatty acid composition;

fertilizing the selected female (milk-producing) cattle using the non-human semen or non-human sperm according to the second aspect of the present invention.

According to other particular embodiments, the at least one allele is an allele of at least one polymorphism. The at least one polymorphism may be selected from the polymorphisms listed in table 1.

According to other particular embodiments, the at least one allele is:

a “non-fat allele” for C16:0 of at least one polymorphism selected from the group consisting of P #33, P #241-P #248, P #303-P #312, P #314-P #344, P #346-P #475 and, P #477-P #481;

a “fat allele” for C18:1 of at least one polymorphism selected from the group consisting of P #19, P #34, P #38, P #39, P #141, P #148, P #153, P #233-P #240, P #242-P #246, P #271-P #278, P #280-P #283, P #285, P #289-P #302, P #311-P #334, P #339, P #340, P #343-P #475, P #477-P #481, P #484-P #494, P #497, P #543, P #588, P #589, P #916, P #911, P #14, P #37, P #903 and P #7;

a “fat allele” for C14:1 cis-9 of at least one polymorphism selected from the group consisting of P #243-P #246, P #314-P #332, P #334-P #340, P #346-P #352, P #354-P #363, P #366-P #392, P #394-P #396, P #398-P #410, P #412-P #475 and P #477-P #481;

a “fat allele” for C6:0 of at least one polymorphism selected from the group consisting of P #1, P #12, P #15-P #17, P #24, P #41, P #63, P #64, P #69-P #71, P #74-P #79, P #83, P #85-P #87, P #89-P #93, P #97-P #103, P #112-P #118, P #120, P #121, P #123-P #135, P #138-P #141, P #148, P #153-P #168, P #218, P #220-P #226, P #249, P #250, P #253-P #260, P #267, P #495, P #499, P #501, P #503-P #514, P #517-P #539, P #541, P #542, P #544-P #547, P #554-P #585, P #590-P #592, P #595-P #597, P #599-P #614, P #617-P #621, P #623-P #652, P #654-P #669, P #672-P #674, P #676-P #678, P #681, P #682, P #688-P #744, P #746-P #769, P #771-P #813, P #815, P #818-P #824, P #826-P #831 and P #872;

a “fat allele” for C8:0 of at least one polymorphism selected from the group consisting of P #1, P #10-P #12, P #16-P #19, P #40-P #45, P #48, P #49, P #51, P #56-P #60, P #63-P #66, P #68-P #93, P #97, P #99-P #101, P #103-P #108, P #111-P #118, P #120, P #121, P #123-P #218, P #220, P #224-P #231, P #254, P #258, P #259, P #268, P #270, P #285-P #288, P #302, P #495, P #496, P #498-P #539, P #541-P #551, P #554-P #586, P #590-P #594, P #628, P #682 and P #683;

a “fat allele” for C10:0 of at least one polymorphism selected from the group consisting of P #3, P #8-P #10, P #16-P #20, P #49-P #51, P #56-P #62, P #64, P #69-P #72, P #74, P #76-P #78, P #80-P #96, P #104-P #109, P #111-P #115, P #117, P #118, P #120, P #121, P #127, P #128, P #132, P #133, P #136-P #142, P #148-P #153, P #156-P #166, P #224-P #226, P #285-P #288, P #297-P #302, P #490-P #494, P #501-P #514, P #517-P #539, P #541-P #547, P #551, P #554-P #580, P #585, P #586, P #589-P #593, P #628, P #889-P #891, P #899, P #900, P #902-P #916;

a “fat allele” for C12:0 of at least one polymorphism selected from the group consisting of P #3, P #9, P #10, P #16-P #20, P #49-P #53, P #61, P #62, P #64, P #67, P #69-P #72, P #74, P #83, P #85-P #87, P #89-P #96, P #104-P #113, P #115, P #117-P #122, P #127, P #128, P #132, P #133, P #138, P #139, P #141, P #142, P #148-P #151, P #153, P #219, P #224-P #226, P #276, P #285-P #288, P #290-P #302, P #487-P #495, P #497, P #501-P #515, P #517-P #547, P #551, P #554-P #580, P #585, P #586, P #588-P #594, P #887-P #891, P #893-P #916;

a “fat allele” for C14:0 of at least one polymorphism selected from the group consisting of P #9, P #10, P #17-P #19, P #34, P #69, P #70, P #83, P #85, P #117, P #120, P #121, P #127, P #128, P #132, P #133, P #139, P #141, P #148-P #151, P #153, P #224-P #226, P #275, P #276, P #279-P #281, P #284-P #302, P #483-P #494, P #497, P #501-P #514, P #517-P #539, P #541, P #543-P #580, P #584-P #594, P #889-P #892, P #899, P #900, P #902-P #916; or

any combination thereof.

According to other particular embodiments, the at least one allele is a “non-fat allele” for C16:0 of at least one polymorphism selected from the group consisting of P #33, P #241-P #248, P #303-P #312, P #314-P #344, P #346-P #475 and, P #477-P #481.

According to particular embodiments, the present invention provides a method for selective breeding of a cattle, the method comprises:

determining the identity of one or more nucleotides of at least one allele of at least one polymorphism, which in a female milk-producing cattle is indicative of decreased amount of C16:0 in milk, within the genome of a (suitable) female (milk-producing) cattle, said at least one polymorphism being located within said genome at a position corresponding to position 60 of the nucleotide sequence set forth in any one of SEQ ID NO 33, 241-248, 303-312, 314-344, 346-475 and 477-481; or at a position corresponding to position 60 of a nucleotide sequence which is derived from any one of SEQ ID NO 33, 241-248, 303-312, 314-344, 346-475 and 477-481 by 1 to 30 nucleotide substitutions; and

selecting said female (milk-producing) cattle when the one or more nucleotides of the at least one allele is one or more nucleotides corresponding to the “non-fat allele” for C16:0;

providing non-human semen or non-human sperm comprising within its genome at least one allele which in a female milk-producing cattle is indicative of a desired milk fatty acid composition; and

fertilizing the selected female (milk-producing) cattle using the non-human semen or non-human sperm.

In Vitro Method for Selective Breeding

The present invention provides in a fourth aspect a method for selective breeding of a cattle, the method comprises:

in vitro fertilizing the non-human ovum according to the second aspect of the present invention using the non-human semen or non-human sperm according to the second aspect of the present invention; and

implanting the in vitro fertilized non-human ovum in the uterus of a female (milk-producing) cattle.

Cattle

The present invention provides in a fifth aspect, a cattle obtainable by the method according to the first aspect of the present invention, the method according to the third aspect of the present invention or the method according to the fourth aspect of the present invention.

The present invention further provides in a sixth aspect, a cattle comprising within its genome at least one allele which in a female milk-producing cattle is indicative of a desired milk fatty acid composition.

The at least one allele may be an allele of at least one polymorphism. The at least one polymorphism may be selected from the polymorphisms listed in table 1.

According to particular embodiments, the at least one allele is:

a “non-fat allele” for C16:0 of at least one polymorphism selected from the group consisting of P #33, P #241-P #248, P #303-P #312, P #314-P #344, P #346-P #475 and, P #477-P #481;

a “fat allele” for C18:1 of at least one polymorphism selected from the group consisting of P #19, P #34, P #38, P #39, P #141, P #148, P #153, P #233-P #240, P #242-P #246, P #271-P #278, P #280-P #283, P #285, P #289-P #302, P #311-P #334, P #339, P #340, P #343-P #475, P #477-P #481, P #484-P #494, P #497, P #543, P #588, P #589, P #916, P #911, P #14, P #37, P #903 and P #7;

a “fat allele” for C14:1 cis-9 of at least one polymorphism selected from the group consisting of P #243-P #246, P #314-P #332, P #334-P #340, P #346-P #352, P #354-P #363, P #366-P #392, P #394-P #396, P #398-P #410, P #412-P #475 and P #477-P #481;

a “fat allele” for C6:0 of at least one polymorphism selected from the group consisting of P #1, P #12, P #15-P #17, P #24, P #41, P #63, P #64, P #69-P #71, P #74-P #79, P #83, P #85-P #87, P #89-P #93, P #97-P #103, P #112-P #118, P #120, P #121, P #123-P #135, P #138-P #141, P #148, P #153-P #168, P #218, P #220-P #226, P #249, P #250, P #253-P #260, P #267, P #495, P #499, P #501, P #503-P #514, P #517-P #539, P #541, P #542, P #544-P #547, P #554-P #585, P #590-P #592, P #595-P #597, P #599-P #614, P #617-P #621, P #623-P #652, P #654-P #669, P #672-P #674, P #676-P #678, P #681, P #682, P #688-P #744, P #746-P #769, P #771-P #813, P #815, P #818-P #824, P #826-P #831 and P #872;

a “fat allele” for C8:0 of at least one polymorphism selected from the group consisting of P #1, P #10-P #12, P #16-P #19, P #40-P #45, P #48, P #49, P #51, P #56-P #60, P #63-P #66, P #68-P #93, P #97, P #99-P #101, P #103-P #108, P #111-P #118, P #120, P #121, P #123-P #218, P #220, P #224-P #231, P #254, P #258, P #259, P #268, P #270, P #285-P #288, P #302, P #495, P #496, P #498-P #539, P #541-P #551, P #554-P #586, P #590-P #594, P #628, P #682 and P #683;

a “fat allele” for C10:0 of at least one polymorphism selected from the group consisting of P #3, P #8-P #10, P #16-P #20, P #49-P #51, P #56-P #62, P #64, P #69-P #72, P #74, P #76-P #78, P #80-P #96, P #104-P #109, P #111-P #115, P #117, P #118, P #120, P #121, P #127, P #128, P #132, P #133, P #136-P #142, P #148-P #153, P #156-P #166, P #224-P #226, P #285-P #288, P #297-P #302, P #490-P #494, P #501-P #514, P #517-P #539, P #541-P #547, P #551, P #554-P #580, P #585, P #586, P #589-P #593, P #628, P #889-P #891, P #899, P #900, P #902-P #916;

a “fat allele” for C12:0 of at least one polymorphism selected from the group consisting of P #3, P #9, P #10, P #16-P #20, P #49-P #53, P #61, P #62, P #64, P #67, P #69-P #72, P #74, P #83, P #85-P #87, P #89-P #96, P #104-P #113, P #115, P #117-P #122, P #127, P #128, P #132, P #133, P #138, P #139, P #141, P #142, P #148-P #151, P #153, P #219, P #224-P #226, P #276, P #285-P #288, P #290-P #302, P #487-P #495, P #497, P #501-P #515, P #517-P #547, P #551, P #554-P #580, P #585, P #586, P #588-P #594, P #887-P #891, P #893-P #916;

a “fat allele” for C14:0 of at least one polymorphism selected from the group consisting of P #9, P #10, P #17-P #19, P #34, P #69, P #70, P #83, P #85, P #117, P #120, P #121, P #127, P #128, P #132, P #133, P #139, P #141, P #148-P #151, P #153, P #224-P #226, P #275, P #276, P #279-P #281, P #284-P #302, P #483-P #494, P #497, P #501-P #514, P #517-P #539, P #541, P #543-P #580, P #584-P #594, P #889-P #892, P #899, P #900, P #902-P #916; or

any combination thereof.

According to other particular embodiments, the at least one allele is a “non-fat allele” for C16:0 of at least one polymorphism selected from the group consisting of P #33, P #241-P #248, P #303-P #312, P #314-P #344, P #346-P #475 and, P #477-P #481.

According to particular embodiments, said cattle comprises within its genome at least one nucleotide sequence selected from the group consisting of a) a nucleotide sequence set forth in any one of SEQ ID NO 33, 241-248, 303-312, 314-344, 346-475 and 477-481; and b) nucleotide sequences which are derived from any one of SEQ ID NO 33, 241-248, 303-312, 314-344, 346-475 and 477-481; by 1 to 30 nucleotide substitutions;

wherein the one or more nucleotides at position 60 of the nucleotide sequence set forth in any one of SEQ ID NO 33, 241-248, 303-312, 314-344, 346-475 and 477-481; corresponds to the “non-fat allele” for C16:0.

In one embodiment according to the fifth or sixth aspect of the present invention, said cattle is a (isolated) male cattle.

In another embodiment according to the fifth or sixth aspect of the present invention, said cattle is a (isolated) female milk-producing cattle.

Milk

A seventh aspect of the present invention, relates to milk produced by the (isolated) female milk-producing cattle according to the fifth or sixth aspect of the present invention.

Use

The present invention provides in an eighth aspect, use of an (isolated) nucleic acid molecule in an in vitro method for determining the presence of at least one allele, which in a female milk-producing cattle is indicative of a desired milk fatty acid composition, within the genome of a cattle;

wherein the (isolated) nucleic acid molecule comprises at least one nucleotide sequence selected from the group consisting of a) a nucleotide sequence set forth in any one of SEQ ID NO: 1 to 916, such as SEQ ID NOs: 1 to 310; b) a nucleotide sequence derived from any one of SEQ ID NO: 1 to 916, such as SEQ ID NOs: 1 to 310, by 1 to 30 nucleotide substitutions; and c) complements to a) and b); the one or more nucleotides at position 60 of said nucleotide sequences being selected from the two alternative forms of the allele to be determined.

The present invention provides in a ninth aspect, use of an (isolated) oligonucleotide in an in vitro method for determining the presence of at least one allele, which in a female milk-producing cattle is indicative of a desired milk fatty acid composition, within the genome of a cattle;

wherein the (isolated) oligonucleotide comprises at least 20 contiguous nucleotides of a nucleotide sequence selected from the group consisting of a) a nucleotide sequence set forth in any one of SEQ ID NO: 1 to 916, such as SEQ ID NOs: 1 to 310; b) a nucleotide sequence derived from any one of SEQ ID NO: 1 to 916, such as SEQ ID NOs: 1 to 310, by 1 to 30 nucleotide substitutions; and c) complements to a) and b); said at least 20 contiguous nucleotides include the one or more nucleotides at position 60 of a) or b); and the one or more nucleotides at position 60 of said nucleotide sequences being selected from the two alternative forms of the allele to be determined.

### DETAILED DESCRIPTION OF THE INVENTION

Unless specifically defined herein, all technical and scientific terms used have the same meaning as commonly understood by a skilled artisan in the fields of genetics, biochemistry, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail.

Where a numerical limit or range is stated herein, the endpoints are included. Also, all values and sub ranges within a numerical limit or range are specifically included as if explicitly written out.

Polymorphisms of the Invention

As used herein, a “polymorphism” is a variation in a genomic sequence. In particular, a polymorphism is an allelic variant that is generally found between individuals of a population. The polymorphism may be a single nucleotide difference present at a locus, or may be an insertion or deletion of one or a few nucleotides at a position of a gene.

As used herein, a “single nucleotide polymorphism” or “SNP” refers to a single base (nucleotide) polymorphism in a DNA sequence among individuals in a population. As such, a single nucleotide polymorphism is characterized by the presence in a population of one or two, three or four nucleotides (i.e. adenine, cytosine, guanine or thymine), typically less than all four nucleotides, at a particular locus in a genome.

As used herein, “polymorphic sequence” refers to a nucleotide sequence including a polymorphic site,

As used herein, a “polymorphic site” is the locus or position within a given sequence at which divergence occurs. Preferred polymorphic sites have at least two alleles, each occurring at frequency greater than 1%, such as greater than 5%. Those skilled in the art will recognize that nucleic acid molecules may be double-stranded molecules and that reference to a particular site on one strand refers, as well, to the corresponding site on a complementary strand. In defining a polymorphic site or allele reference to an adenine, a thymine, a cytosine, or a guanine at a particular site on one strand of a nucleic acid molecule also defines the thymine, adenine, guanine, or cytosine (respectively) at the corresponding site on a complementary strand of the nucleic acid.

The present inventors have identified quantitative trait locus (QTL) responsible for at least some of the genetic variation in milk fatty acid composition in female milk-producing Norwegian Red cattle. More specifically, the present inventors have identified polymorphisms within the genome, more particularly on chromosome 1, 4, 5, 6, 10, 11, 12, 13, 15, 17, 19, 23, 26 and 27 of Norwegian Red cattle which are associated with altered milk fatty acid composition in female milk-producing Norwegian Red cattle. Specific details of polymorphisms of the invention are provided in table 1 and table 2 below. The respective nucleotide sequences including the polymorphism at position 60 are shown in Table 2.

The polymorphism of the present invention can be present in either of two forms, i.e., the polymorphisms have a total of two alleles.

For the polymorphisms influencing only one trait (e.g. P #1), one allele can be characterized as being a “fat allele” and the other can be characterized as being a “non-fat allele”. A “fat allele” is associated with an increase in the amount of the fatty acid to which it relates while a “non-fat allele” is associated with a decrease in the amount of the fatty acid to which it relates, i.e. a “fat allele” for C18:1 is associated with an increase in the amount of C18:1 while a “non-fat allele” for C18:1 is associated with a decrease in the amount of C18:1. Thus, a female milk-producing cattle having a “fat allele” at the position of a polymorphism detailed herein provides milk with increased amount of the fatty acid to which the “fat allele” relates while a female milk-producing cattle having a “non-fat allele” at the position of a polymorphism detailed herein provides milk with decreased amount of the fatty acid to which the “fat allele” relates.

For the polymorphisms influencing more than one trait, one polymorphism allele may actually represent a “fat allele” for some of the traits and “non-fat allele” for the others. In order to clarify this even further, reference is made to P #15 where an adenine at the position of the polymorphic site is a “fat allele” for C4:0 and C6:0 while being a “non-fat allele” for CLA. Thus, a female milk-producing cattle having an adenine at the position of the polymorphic site of P #15 provides milk with increased amounts of C4:0 and C6:0 while the amount of CLA is decreased. On the other hand, a female milk-producing cattle having a guanine at the position of the polymorphic site of P #15 provides milk with decreased amounts of C4:0 and C6:0 while the amount of CLA is increased.

Since some of the polymorphisms according to the present invention influence only one trait while others influence more than one trait, the term “a polymorphism allele” is herein meant to refer to one of the two alternative forms of the polymorphism. Said in other words, P #15 has a total of two polymorphism alleles (A/G), i.e. there may be an adenine at the polymorphic site or a guanine at the polymorphic site. An adenine at the polymorphic site represents one polymorphism allele while a guanine at the polymorphic site represents the other polymorphism allele.

Nearly all mammals, including non-human mammals such as cattle, in particular Norwegian Red cattle, are diploid organisms and thus possess at least one copy of the polymorphisms of the invention.

As demonstrated herein, if at least one allele of a polymorphism is the respective “fat-allele” for a specific trait; then a female milk-producing cattle will be able to provide milk with increased content of the trait as compared to a female milk-producing cattle where both alleles are “non-fat allele” for the same trait (homozygous). It would be expected that milk from a female milk-producing cattle being homozygous for a “fat-allele” for a specific trait will contain higher amounts of that trait as compared to milk from a female milk-producing cattle being heterozygous for the “fat-allele” for the same specific trait.

A polymorphism of the invention may be any of several polymorphisms indicative of altered milk fatty acid composition in female milk-producing cattle. Particularly, a polymorphism of the invention is a polymorphism located on chromosome 1, 4, 5, 6, 10, 11, 12, 13, 15, 17, 19, 23, 26 and/or 27 of a cattle, i.e. a polymorphism found to be located on chromosome 1, 4, 5, 6, 10, 11, 12, 13, 15, 17, 19, 23, 26 and/or 27 on the basis of genetic linkage analysis, Fluorescence in situ Hybridization (FISH) or any other method that assigns DNA polymorphisms to their respective chromosomes.

As used herein, “genetic linkage analysis” refers to a statistical procedure where genotype data, coming from sets of animals comprising parents and their offspring, are investigated in order to test for the presence of genetic linkage between polymorphisms. Genetic linkage analysis can be used in order to assign polymorphisms to chromosomes, provided that the analysis incorporates polymorphisms that have already been assigned to chromosome using e.g.

Fluorescence In Situ Hybridiation.

As used herein “Fluorescence In Situ Hybridiation” or “FISH” refers to a technique that detect the presence or absence of specific DNA sequences on chromosomes. FISH can be used in order to assign known DNA polymorphisms to chromosomes.

As used herein, “genetic linkage” refers to the tendency of polymorphisms that are located close to each other on a chromosome to be inherited together during meiosis. Thus, polymorphisms located close to each other on the same chromosome are said to be genetically linked. Alleles at two such genetically linked loci are co-inherited (from parents to offspring) more often than they are not. Assume, for example, two polymorphisms; polymorphism A having alleles A1 and A2, and polymorphism B having alleles B1 and B2. Assume further that a given cattle carries all of the alleles A1, A2, B1, and B2 (in other words, this cattle is heterozygous at both marker and marker B). If alleles A1 and B1 are, in this particular cattle, located on the same chromosome copy, then alleles A1 and B1 are co-inherited, to the offspring of the cattle, more often than not.

The unit “centiMorgan” is a unit of measurement, used to describe genetic distances, where genetic distance is a measure of the extent to which two polymorphisms are genetically linked.

A polymorphism of the invention may be any polymorphism, including single nucleotide polymorphism, which is in strong linkage disequilibrium (LD) with a polymorphism selected from the group consisting of P #1 to P #916. Here, two polymorphisms are defined to be in strong LD if the square of the correlation coefficient between the two loci (r2, the most commonly used measure of LD) is equal to or larger than 0.7 such as equal to or larger than 0.75. A person who is skilled in the art will know how to estimate r2, as well as what data material is required for this estimation.

Linkage disequilibrium (LD) or, more precisely, gametic phase linkage disequilibrium is used in order to describe the co-inheritance of alleles at genetically linked polymorphisms, at the population level. Assume, for example, two polymorphisms located on the same chromosome; polymorphism A having alleles A1 and A2, and polymorphism B having alleles B1 and B2. All copies of the chromosome in question will harbor a combination of alleles at the two loci (i.e. a haplotype), and there are four possible haplotypes: A1-B1, A1-B2, A2-B1, and A2-B2. The two loci are in said to be LD with each other if the number of A1-B1 and A2-B2 haplotypes within the population are significantly larger or significantly smaller than the number of A1-B2 and A2-B1 haplotypes.

A polymorphism of the invention may be at least one of the polymorphisms listed in Table 1. Therefore, according to certain embodiments, the at least one polymorphism of the invention is selected from the polymorphisms listed in Table 1. Each of the polymorphisms listed in Table 1 is contemplated as being disclosed individually as part of the present invention.

TABLE 1
Polymorphisms indicative of altered milk fatty acid composition. P# is the
number of the polymorphism according to the present invention. Trait refers to one
or more fatty acid that is under the influence of the polymorphism in question. C4:0
is butyric acid, C6:0 is caproic acid, C8:0 is octanoic acid, C10:0 is decanoic acid,
C12:0 is dodecanoic acid, C14:0 is tetradecanoic acid, C14:1cis-9 is myristoleic
acid, C16:0 is hexadecanoic acid, C18:1 is Oleic acid, C18:1cis-9, C18:1cis-10,
C18:1cis-11 and C18:1trans-9 are different isomers of Oleic acid. CLA is
conjugated linoleic acid, DHA is docosahexaenoic acid, DNS represents de novo-
synthesized acids (i.e., C6:0 to C16:0), MUFA is monounsaturated acids, NEFA are
free fatty acids, SAT are saturated fatty acids. BTA# is the chromosome to which
the polymorphism is positioned. The Ref# is the ID number of the polymorphism
from the Single Nucleotide Polymorphism database. Position (bp) is the
chromosome position from the UMD 3.1 assembly (NCBI assembly
accession GCA_000003055.4). Those skilled in the art may easily identify the
reference sequence and the position of the polymorphism within the NCBI (National
Center for Biotechnology Information) Single Nucleotide Polymorphism Database.
A = Adenine, G = Guanine; C = Cytosine, T = Thymine. “Fat allele” indicates the allele
that is associated with an increase in the amount of the respective trait. “Non-Fat
allele” indicates the allele that is associated with a decrease in the amount of the
respective trait. The P# and the SEQIDNO are identical.

| P#/ | | | | Position | | Non-fat |
|---|---|---|---|---|---|---|
| SEQIDNO | Trait | BTA # | Ref # | (bp) | Fat allele | allele |
| 1 | C8:0 | 1 | rs29027221 | 126338729 | A | C |
| | C6:0 | | | | A | C |
| 2 | DHA | 1 | rs41639338 | 126596653 | A | C |
| 3 | DNS | 1 | rs29021652 | 131979848 | A | G |
| | C12:0 | | | | A | G |
| | C10:0 | | | | A | G |
| 4 | CLA | 4 | rs41650703 | 48649499 | A | T |
| 5 | DHA | 4 | rs41612156 | 49857470 | A | G |
| 6 | CLA | 5 | rs41611305 | 91471989 | T | C |
| | SAT | | | | C | T |
| 7 | C18:1trans-9 | 6 | rs29024684 | 87396306 | C | A |
| 8 | C10:0 | 10 | rs43707537 | 36369609 | A | G |
| | DNS | | | | A | G |
| 9 | DNS | 10 | rs41623983 | 38738818 | T | A |
| | C14:0 | | | | A | T |
| | C12:0 | | | | A | T |
| | C10:0 | | | | A | T |
| 10 | DNS | 10 | rs41623991 | 38968764 | G | A |
| | C8:0 | | | | G | A |
| | C14:0 | | | | G | A |
| | C12:0 | | | | G | A |
| | C10:0 | | | | G | A |
| 11 | C8:0 | 10 | rs43707029 | 40424349 | G | A |
| 12 | C8:0 | 10 | s43707539 | 43031877 | G | A |
| | C6:0 | | | | G | A |
| 13 | C4:0 | 10 | rs41568266 | 46642818 | G | A |
| 14 | C18:1cis-11 | 12 | s41569014 | 88316926 | C | T |
| 15 | C4:0 | 13 | rs29012218 | 55409520 | A | G |
| | C6:0 | | | | A | G |
| | CLA | | | | G | A |
| 16 | DNS | 13 | rs41631605 | 64500844 | G | A |
| | C8:0 | | | | G | A |
| | C6:0 | | | | G | A |
| | C10:0 | | | | G | A |
| | C12:0 | | | | G | A |
| 17 | DNS | 13 | rs29018443 | 64841951 | C | T |
| | C8:0 | | | | C | T |
| | C6:0 | | | | C | T |
| | C14:0 | | | | C | T |
| | C12:0 | | | | C | T |
| | C10:0 | | | | C | T |
| 18 | DNS | 13 | rs41631563 | 66080035 | C | A |
| | C8:0 | | | | C | A |
| | C14:0 | | | | C | A |
| | C12:0 | | | | C | A |
| | C10:0 | | | | C | A |
| 19 | C10:0 | 15 | s41582050 | 28098734 | T | C |
| | C12:0 | | | | T | C |
| | C14:0 | | | | T | C |
| | C18:1cis-9 | | | | C | T |
| | C8:0 | | | | T | C |
| | DNS | | | | T | C |
| 20 | C12:0 | 15 | rs41622342 | 64373612 | G | A |
| | C10:0 | | | | A | G |
| 21 | C4:0 | 17 | rs41637576 | 40693717 | A | G |
| 22 | C4:0 | 17 | rs41637616 | 43463045 | A | G |
| 23 | C4:0 | 17 | rs41637627 | 45145609 | A | C |
| 24 | C6:0 | 17 | rs41636968 | 45302499 | C | T |
| | C4:0 | | | | C | T |
| 25 | C4:0 | 17 | rs41585556 | 45632698 | C | A |
| 26 | C4:0 | 17 | rs41638773 | 47179691 | G | C |
| 27 | C4:0 | 17 | rs41633197 | 47684549 | A | G |
| 28 | C4:0 | 17 | rs41666579 | 47995988 | A | G |
| 29 | C4:0 | 17 | rs41638780 | 49227724 | A | G |
| 30 | C4:0 | 17 | rs41661878 | 51024797 | G | A |
| 31 | C4:0 | 17 | rs41660449 | 53592758 | G | T |
| 32 | C4:0 | 17 | rs41660450 | 53620065 | C | A |
| 33 | C16:0 | 17 | s41630100 | 59778912 | G | A |
| 34 | SAT | 17 | rs41576270 | 61082159 | A | G |
| | MUFA | | | | G | A |
| | C18:1cis-9 | | | | G | A |
| | C14:0 | | | | A | G |
| 35 | SAT | 17 | rs41634411 | 72384103 | A | G |
| 36 | NEFA | 23 | rs29019650 | 11907305 | A | C |
| 37 | C18:1cis-11 | 23 | rs41617401 | 45507722 | C | A |
| 38 | SAT | 26 | rs41664843 | 27325539 | T | A |
| | C18:1cis-9 | | | | A | T |
| 39 | SAT | 26 | rs41636621 | 28096889 | C | G |
| | MUFA | | | | G | C |
| | C18:1cis-9 | | | | G | C |
| 40 | C8:0 | 13 | rs42476347 | 36576754 | C | T |
| 41 | C6:0 | 13 | rs41577018 | 36579808 | G | T |
| | C8:0 | | | | G | T |
| 42 | C8:0 | 13 | rs29016293 | 36591954 | T | C |
| 43 | C8:0 | 13 | rs109346582 | 39102096 | T | C |
| 44 | C8:0 | 13 | rs43259281 | 39542292 | T | G |
| | DNS | | | | T | G |
| 45 | C8:0 | 13 | rs135689087 | 40632008 | T | C |
| 46 | C4:0 | 13 | rs43434744 | 46062881 | C | T |
| 47 | C4:0 | 13 | rs41696754 | 55421364 | C | T |
| 48 | C8:0 | 13 | rs41698815 | 58523914 | G | A |
| 49 | DNS | 13 | rs110795124 | 59579696 | A | G |
| | C8:0 | | | | A | G |
| | C12:0 | | | | A | G |
| | C10:0 | | | | A | G |
| 50 | C12:0 | 13 | rs41701362 | 60112222 | A | C |
| | C10:0 | | | | A | C |
| 51 | C10:0 | 13 | rs133849041 | 60507659 | G | T |
| | C12:0 | | | | G | T |
| | C8:0 | | | | G | T |
| | DNS | | | | G | T |
| 52 | C12:0 | 13 | rs41705197 | 60515732 | G | T |
| 53 | C12:0 | 13 | rs135049699 | 60521218 | A | C |
| 54 | C4:0 | 13 | rs132915254 | 60527253 | G | A |
| 55 | C4:0 | 13 | rs41699542 | 60570356 | G | A |
| 56 | C10:0 | 13 | rs109785720 | 60627246 | C | T |
| | C8:0 | | | | C | T |
| | DNS | | | | C | T |
| 57 | DNS | 13 | rs109565416 | 60628472 | G | A |
| | C8:0 | | | | G | A |
| | C10:0 | | | | G | A |
| 58 | C10:0 | 13 | rs41576886 | 60643827 | C | T |
| | C8:0 | | | | C | T |
| | DNS | | | | C | T |
| 59 | DNS | 13 | rs132981660 | 60688808 | A | C |
| | C8:0 | | | | A | C |
| | C10:0 | | | | A | C |
| 60 | C10:0 | 13 | rs41703753 | 60691132 | C | T |
| | C8:0 | | | | C | T |
| | DNS | | | | C | T |
| 61 | C12:0 | 13 | rs110362163 | 60695568 | A | G |
| | C10:0 | | | | A | G |
| 62 | C12:0 | 13 | rs133399057 | 60873458 | G | A |
| | C10:0 | | | | G | A |
| 63 | C8:0 | 13 | rs109001485 | 61902355 | A | G |
| | C6:0 | | | | A | G |
| 64 | C10:0 | 13 | rs109396998 | 61984979 | C | T |
| | C12:0 | | | | C | T |
| | C6:0 | | | | C | T |
| | C8:0 | | | | C | T |
| | DNS | | | | C | T |
| 65 | C8:0 | 13 | rs136665696 | 62065597 | C | T |
| | DNS | | | | C | T |
| 66 | DNS | 13 | rs109446607 | 62067270 | A | G |
| | C8:0 | | | | A | G |
| 67 | C12:0 | 13 | rs109256322 | 62068335 | A | G |
| 68 | C8:0 | 13 | rs132702901 | 62069070 | C | T |
| | DNS | | | | C | T |
| 69 | DNS | 13 | rs109995538 | 62448949 | G | A |
| | C8:0 | | | | G | A |
| | C6:0 | | | | G | A |
| | C12:0 | | | | G | A |
| | C10:0 | | | | G | A |
| | C14:0 | | | | G | A |
| 70 | C10:0 | 13 | rs110285330 | 62450667 | T | G |
| | C12:0 | | | | T | G |
| | C6:0 | | | | T | G |
| | C8:0 | | | | T | G |
| | DNS | | | | T | G |
| | C14:0 | | | | T | G |
| 71 | DNS | 13 | rs110309401 | 62527818 | A | G |
| | C8:0 | | | | A | G |
| | C6:0 | | | | A | G |
| | C12:0 | | | | A | G |
| | C10:0 | | | | A | G |
| 72 | DNS | 13 | rs41706412 | 62528401 | A | C |
| | C8:0 | | | | A | C |
| | C12:0 | | | | A | C |
| | C10:0 | | | | A | C |
| 73 | C8:0 | 13 | rs43206444 | 62553264 | C | T |
| | DNS | | | | C | T |
| 74 | DNS | 13 | rs41707249 | 62556854 | G | A |
| | C8:0 | | | | G | A |
| | C6:0 | | | | G | A |
| | C12:0 | | | | G | A |
| | C10:0 | | | | G | A |
| 75 | C6:0 | 13 | rs134395454 | 62719766 | T | C |
| | C8:0 | | | | T | C |
| | DNS | | | | T | C |
| 76 | DNS | 13 | rs136727738 | 62727770 | A | G |
| | C8:0 | | | | A | G |
| | C6:0 | | | | A | G |
| | C10:0 | | | | A | G |
| 77 | C10:0 | 13 | rs41700824 | 62836175 | C | T |
| | C6:0 | | | | C | T |
| | C8:0 | | | | C | T |
| | DNS | | | | C | T |
| 78 | DNS | 13 | rs41701735 | 62858269 | A | G |
| | C8:0 | | | | A | G |
| | C6:0 | | | | A | G |
| | C10:0 | | | | A | G |
| 79 | C6:0 | 13 | rs136662836 | 62885520 | C | T |
| | C8:0 | | | | C | T |
| 80 | C8:0 | 13 | rs110784592 | 62916334 | A | G |
| | C10:0 | | | | A | G |
| 81 | C8:0 | 13 | rs109587232 | 62925849 | A | G |
| | C10:0 | | | | A | G |
| 82 | C10:0 | 13 | rs110489811 | 62928756 | T | C |
| | C8:0 | | | | T | C |
| 83 | C10:0 | 13 | rs109862645 | 62933067 | G | T |
| | C12:0 | | | | G | T |
| | C6:0 | | | | G | T |
| | C8:0 | | | | G | T |
| | DNS | | | | G | T |
| | C14:0 | | | | G | T |
| 84 | C10:0 | 13 | rs109018242 | 62942668 | T | C |
| | C8:0 | | | | T | C |
| 85 | DNS | 13 | rs133076301 | 62944250 | G | A |
| | C8:0 | | | | G | A |
| | C6:0 | | | | G | A |
| | C12:0 | | | | G | A |
| | C10:0 | | | | G | A |
| | C14:0 | | | | G | A |
| 86 | C10:0 | 13 | rs109518939 | 62947465 | C | T |
| | C12:0 | | | | C | T |
| | C6:0 | | | | C | T |
| | C8:0 | | | | C | T |
| | DNS | | | | C | T |
| 87 | DNS | 13 | rs110743763 | 62950126 | A | G |
| | C8:0 | | | | A | G |
| | C6:0 | | | | A | G |
| | C12:0 | | | | A | G |
| | C10:0 | | | | A | G |
| 88 | C10:0 | 13 | rs41602440 | 62951257 | T | C |
| | C8:0 | | | | T | C |
| 89 | C10:0 | 13 | rs110608478 | 62953893 | G | T |
| | C12:0 | | | | G | T |
| | C6:0 | | | | G | T |
| | C8:0 | | | | G | T |
| | DNS | | | | G | T |
| 90 | C10:0 | 13 | rs110813518 | 62954588 | T | G |
| | C12:0 | | | | T | G |
| | C6:0 | | | | T | G |
| | C8:0 | | | | T | G |
| | DNS | | | | T | G |
| 91 | C10:0 | 13 | rs110767129 | 62955647 | C | T |
| | C12:0 | | | | C | T |
| | C6:0 | | | | C | T |
| | C8:0 | | | | C | T |
| | DNS | | | | C | T |
| 92 | DNS | 13 | rs110671382 | 62956547 | A | G |
| | C8:0 | | | | A | G |
| | C6:0 | | | | A | G |
| | C12:0 | | | | A | G |
| | C10:0 | | | | A | G |
| 93 | C10:0 | 13 | rs108989331 | 62960536 | T | C |
| | C12:0 | | | | T | C |
| | C6:0 | | | | T | C |
| | C8:0 | | | | T | C |
| | DNS | | | | T | C |
| 94 | C12:0 | 13 | rs110858883 | 62967761 | C | A |
| | C10:0 | | | | C | A |
| 95 | C10:0 | 13 | rs109248916 | 62969467 | T | C |
| | C12:0 | | | | T | C |
| 96 | C12:0 | 13 | rs108958622 | 62971963 | A | G |
| | C10:0 | | | | A | G |
| 97 | C8:0 | 13 | rs41624061 | 62987632 | A | G |
| | C6:0 | | | | A | G |
| 98 | C6:0 | 13 | rs135592700 | 63079589 | A | G |
| 99 | C6:0 | 13 | rs109116663 | 63085508 | T | G |
| | C8:0 | | | | T | G |
| 100 | C6:0 | 13 | rs109001474 | 63093719 | C | T |
| | C8:0 | | | | C | T |
| 101 | C8:0 | 13 | rs110684557 | 63096370 | G | A |
| | C6:0 | | | | G | A |
| 102 | C6:0 | 13 | rs133997573 | 63109630 | T | C |
| 103 | C6:0 | 13 | rs109083431 | 63115088 | T | C |
| | C8:0 | | | | T | C |
| 104 | DNS | 13 | rs137086701 | 63142046 | G | A |
| | C8:0 | | | | G | A |
| | C12:0 | | | | G | A |
| | C10:0 | | | | G | A |
| 105 | DNS | 13 | rs135714488 | 63144732 | A | G |
| | C8:0 | | | | A | G |
| | C12:0 | | | | A | G |
| | C10:0 | | | | A | G |
| 106 | DNS | 13 | rs136873029 | 63146420 | G | A |
| | C8:0 | | | | G | A |
| | C12:0 | | | | G | A |
| | C10:0 | | | | G | A |
| 107 | DNS | 13 | rs135016598 | 63146983 | C | A |
| | C8:0 | | | | C | A |
| | C12:0 | | | | C | A |
| | C10:0 | | | | C | A |
| 108 | C10:0 | 13 | rs133539257 | 63152787 | T | C |
| | C12:0 | | | | T | C |
| | C8:0 | | | | T | C |
| | DNS | | | | T | C |
| 109 | C12:0 | 13 | rs110667975 | 63157239 | A | G |
| | C10:0 | | | | A | G |
| 110 | C12:0 | 13 | rs110377320 | 63163288 | A | G |
| 111 | DNS | 13 | rs133516432 | 63173874 | G | A |
| | C8:0 | | | | G | A |
| | C12:0 | | | | G | A |
| | C10:0 | | | | G | A |
| 112 | DNS | 13 | rs133227293 | 63235326 | A | G |
| | C8:0 | | | | A | G |
| | C6:0 | | | | A | G |
| | C12:0 | | | | A | G |
| | C10:0 | | | | A | G |
| 113 | C10:0 | 13 | rs109626824 | 63236944 | T | C |
| | C12:0 | | | | T | C |
| | C6:0 | | | | T | C |
| | C8:0 | | | | T | C |
| | DNS | | | | T | C |
| 114 | DNS | 13 | rs135705002 | 63238798 | A | G |
| | C8:0 | | | | A | G |
| | C6:0 | | | | A | G |
| | C10:0 | | | | A | G |
| 115 | DNS | 13 | rs41631546 | 63239686 | A | C |
| | C8:0 | | | | A | C |
| | C6:0 | | | | A | C |
| | C12:0 | | | | A | C |
| | C10:0 | | | | A | C |
| 116 | C8:0 | 13 | rs41631542 | 63242901 | A | G |
| | C6:0 | | | | A | G |
| 117 | DNS | 13 | rs137753247 | 63267570 | G | A |
| | C8:0 | | | | G | A |
| | C6:0 | | | | G | A |
| | C12:0 | | | | G | A |
| | C10:0 | | | | G | A |
| | C14:0 | | | | G | A |
| 118 | C10:0 | 13 | rs110620812 | 63428186 | C | T |
| | C12:0 | | | | C | T |
| | C6:0 | | | | C | T |
| | C8:0 | | | | C | T |
| | DNS | | | | C | T |
| 119 | C12:0 | 13 | rs41616451 | 63715266 | T | C |
| 120 | C10:0 | 13 | rs41707948 | 63843713 | T | C |
| | C12:0 | | | | T | C |
| | C6:0 | | | | T | C |
| | C8:0 | | | | T | C |
| | DNS | | | | T | C |
| | C14:0 | | | | T | C |
| 121 | DNS | 13 | rs41707954 | 63848241 | A | G |
| | C8:0 | | | | A | G |
| | C6:0 | | | | A | G |
| | C12:0 | | | | A | G |
| | C10:0 | | | | A | G |
| 122 | C12:0 | 13 | rs136064116 | 64028977 | C | T |
| 123 | C6:0 | 13 | rs134716771 | 64150567 | T | C |
| | C8:0 | | | | T | C |
| 124 | C6:0 | 13 | rs134107495 | 64220819 | C | T |
| | C8:0 | | | | C | T |
| 125 | C8:0 | 13 | rs137438204 | 64226872 | A | G |
| | C6:0 | | | | A | G |
| 126 | C8:0 | 13 | rs132924857 | 64228423 | A | G |
| | C6:0 | | | | A | G |
| 127 | C10:0 | 13 | rs134956071 | 64349171 | C | T |
| | C6:0 | | | | C | T |
| | C8:0 | | | | C | T |
| | DNS | | | | C | T |
| | C12:0 | | | | C | T |
| | C14:0 | | | | C | T |
| 128 | DNS | 13 | rs132687845 | 64354283 | G | A |
| | C8:0 | | | | G | A |
| | C6:0 | | | | G | A |
| | C12:0 | | | | G | A |
| | C10:0 | | | | G | A |
| | C14:0 | | | | G | A |
| 129 | C8:0 | 13 | rs135163820 | 64366788 | A | G |
| | C6:0 | | | | A | G |
| 130 | C8:0 | 13 | rs134080061 | 64398138 | G | A |
| | C6:0 | | | | G | A |
| 131 | C8:0 | 13 | rs29014977 | 64399733 | T | C |
| | C6:0 | | | | T | C |
| 132 | C10:0 | 13 | rs137134199 | 64411593 | C | T |
| | C6:0 | | | | C | T |
| | C8:0 | | | | C | T |
| | DNS | | | | C | T |
| | C12:0 | | | | C | T |
| | C14:0 | | | | C | T |
| 133 | C10:0 | 13 | rs109718644 | 64429162 | C | T |
| | C6:0 | | | | C | T |
| | C8:0 | | | | C | T |
| | DNS | | | | C | T |
| | C12:0 | | | | C | T |
| | C14:0 | | | | C | T |
| 134 | C8:0 | 13 | rs109390067 | 64432139 | A | G |
| | C6:0 | | | | A | G |
| 135 | C8:0 | 13 | rs136344221 | 64453214 | G | A |
| | C6:0 | | | | G | A |
| 136 | DNS | 13 | rs110904633 | 64478327 | A | G |
| | C8:0 | | | | A | G |
| | C10:0 | | | | A | G |
| 137 | DNS | 13 | rs109739218 | 64480379 | G | A |
| | C8:0 | | | | G | A |
| | C10:0 | | | | G | A |
| 138 | DNS | 13 | rs136096188 | 64496757 | G | A |
| | C8:0 | | | | G | A |
| | C6:0 | | | | G | A |
| | C12:0 | | | | G | A |
| | C10:0 | | | | G | A |
| 139 | C10:0 | 13 | rs43771381 | 64589486 | T | C |
| | C12:0 | | | | T | C |
| | C6:0 | | | | T | C |
| | C8:0 | | | | T | C |
| | DNS | | | | T | C |
| | C14:0 | | | | T | C |
| 140 | DNS | 13 | rs41700748 | 64621429 | G | A |
| | C8:0 | | | | G | A |
| | C6:0 | | | | G | A |
| | C10:0 | | | | G | A |
| 141 | C10:0 | 13 | rs41700740 | 64650276 | T | C |
| | C12:0 | | | | T | C |
| | C6:0 | | | | T | C |
| | C8:0 | | | | T | C |
| | DNS | | | | T | C |
| | C14:0 | | | | T | C |
| | C18:1cis-9 | | | | C | T |
| 142 | DNS | 13 | rs43717470 | 64795019 | G | A |
| | C8:0 | | | | G | A |
| | C12:0 | | | | G | A |
| | C10:0 | | | | G | A |
| 143 | DNS | 13 | rs43717461 | 64804947 | C | A |
| | C8:0 | | | | C | A |
| 144 | C8:0 | 13 | rs43717457 | 64831414 | C | T |
| | DNS | | | | C | T |
| 145 | C8:0 | 13 | rs43708452 | 64839762 | T | C |
| | DNS | | | | T | C |
| 146 | DNS | 13 | rs43717453 | 64840460 | C | A |
| | C8:0 | | | | C | A |
| 147 | DNS | 13 | rs43717439 | 64865042 | G | A |
| | C8:0 | | | | G | A |
| 148 | DNS | 13 | rs43711970 | 65246092 | A | G |
| | C8:0 | | | | A | G |
| | C6:0 | | | | A | G |
| | C12:0 | | | | A | G |
| | C10:0 | | | | A | G |
| | C14:0 | | | | A | G |
| | C18:1cis-9 | | | | G | A |
| 149 | C10:0 | 13 | rs110318473 | 65250364 | T | C |
| | C12:0 | | | | T | C |
| | C14:0 | | | | T | C |
| | C8:0 | | | | T | C |
| | DNS | | | | T | C |
| 150 | C12:0 | 13 | rs109153523 | 65255565 | G | A |
| | C10:0 | | | | G | A |
| | C14:0 | | | | G | A |
| | C8:0 | | | | G | A |
| | DNS | | | | G | A |
| 151 | C10:0 | 13 | rs133947598 | 65274414 | C | T |
| | C8:0 | | | | C | T |
| | C12:0 | | | | C | T |
| | C14:0 | | | | C | T |
| | DNS | | | | C | T |
| 152 | C8:0 | 13 | rs43712870 | 65283149 | G | A |
| | C10:0 | | | | G | A |
| | DNS | | | | G | A |
| 153 | DNS | 13 | rs43712867 | 65285989 | G | A |
| | C8:0 | | | | G | A |
| | C6:0 | | | | G | A |
| | C12:0 | | | | G | A |
| | C10:0 | | | | G | A |
| | C14:0 | | | | G | A |
| | C18:1cis-9 | | | | A | G |
| 154 | C8:0 | 13 | rs132985406 | 66563331 | G | A |
| | C6:0 | | | | G | A |
| 155 | C6:0 | 13 | rs132795765 | 66913824 | C | T |
| | C8:0 | | | | C | T |
| 156 | C10:0 | 13 | rs41700666 | 67463110 | T | C |
| | C6:0 | | | | T | C |
| | C8:0 | | | | T | C |
| | DNS | | | | T | C |
| 157 | DNS | 13 | rs109518616 | 67464466 | G | A |
| | C8:0 | | | | G | A |
| | C6:0 | | | | G | A |
| | C10:0 | | | | G | A |
| 158 | C10:0 | 13 | rs110730675 | 67465157 | T | C |
| | C6:0 | | | | T | C |
| | C8:0 | | | | T | C |
| | DNS | | | | T | C |
| 159 | DNS | 13 | rs109963351 | 67465890 | G | A |
| | C8:0 | | | | G | A |
| | C6:0 | | | | G | A |
| | C10:0 | | | | G | A |
| 160 | DNS | 13 | rs110439451 | 67466746 | A | G |
| | C8:0 | | | | A | G |
| | C6:0 | | | | A | G |
| | C10:0 | | | | A | G |
| 161 | DNS | 13 | rs109452111 | 67467905 | G | A |
| | C8:0 | | | | G | A |
| | C6:0 | | | | G | A |
| | C10:0 | | | | G | A |
| 162 | C10:0 | 13 | rs110654124 | 67468746 | G | T |
| | C6:0 | | | | G | T |
| | C8:0 | | | | G | T |
| | DNS | | | | G | T |
| 163 | C10:0 | 13 | rs41700654 | 67469211 | C | T |
| | C6:0 | | | | C | T |
| | C8:0 | | | | C | T |
| | DNS | | | | C | T |
| 164 | DNS | 13 | rs41700644 | 67472330 | A | C |
| | C8:0 | | | | A | C |
| | C6:0 | | | | A | C |
| | C10:0 | | | | A | C |
| 165 | DNS | 13 | rs41700641 | 67473098 | A | G |
| | C8:0 | | | | A | G |
| | C6:0 | | | | A | G |
| | C10:0 | | | | A | G |
| 166 | DNS | 13 | rs109703974 | 67481524 | G | A |
| | C8:0 | | | | G | A |
| | C6:0 | | | | G | A |
| | C10:0 | | | | G | A |
| 167 | C6:0 | 13 | rs133268469 | 67697067 | C | T |
| | C8:0 | | | | C | T |
| | DNS | | | | C | T |
| 168 | C8:0 | 13 | rs133086737 | 67932600 | C | A |
| | C6:0 | | | | C | A |
| 169 | C8:0 | 13 | rs133249272 | 68348045 | G | A |
| 170 | DNS | 13 | rs110626829 | 68349659 | G | A |
| | C8:0 | | | | G | A |
| 171 | C8:0 | 13 | rs109374207 | 68354415 | C | T |
| 172 | C8:0 | 13 | rs109972468 | 68355260 | T | C |
| 173 | C8:0 | 13 | rs134624250 | 68357436 | G | A |
| 174 | C8:0 | 13 | rs109084883 | 68359199 | T | C |
| 175 | C8:0 | 13 | rs134805509 | 68361570 | C | T |
| 176 | C8:0 | 13 | rs109769171 | 68362587 | A | G |
| 177 | C8:0 | 13 | rs133781741 | 68364357 | T | C |
| | DNS | | | | T | C |
| 178 | C8:0 | 13 | rs109399712 | 68370273 | A | G |
| 179 | C8:0 | 13 | rs109404095 | 68375092 | A | G |
| 180 | C8:0 | 13 | rs109590629 | 68376188 | T | C |
| | DNS | | | | T | C |
| 181 | C8:0 | 13 | rs110486144 | 68379681 | T | C |
| | DNS | | | | T | C |
| 182 | C8:0 | 13 | rs133277229 | 68399388 | C | T |
| 183 | C8:0 | 13 | rs135144073 | 68419080 | G | T |
| 184 | C8:0 | 13 | rs136438584 | 68422769 | C | T |
| 185 | C8:0 | 13 | rs109162250 | 68425212 | A | C |
| 186 | C8:0 | 13 | rs135924615 | 68433715 | T | C |
| 187 | C8:0 | 13 | rs110112896 | 68435354 | A | G |
| 188 | C8:0 | 13 | rs108943806 | 68436084 | A | G |
| 189 | C8:0 | 13 | rs29027215 | 68436648 | G | A |
| 190 | C8:0 | 13 | rs29027216 | 68437003 | T | C |
| 191 | C8:0 | 13 | rs110456460 | 68437969 | T | C |
| 192 | C8:0 | 13 | rs109738599 | 68443709 | T | C |
| 193 | C8:0 | 13 | rs110322269 | 68444812 | G | A |
| 194 | C8:0 | 13 | rs110931262 | 68446011 | G | A |
| 195 | C8:0 | 13 | rs109583653 | 68448206 | A | C |
| 196 | C8:0 | 13 | rs109860180 | 68448900 | T | C |
| 197 | C8:0 | 13 | rs108999453 | 68449455 | A | C |
| 198 | C8:0 | 13 | rs110200669 | 68452106 | A | G |
| 199 | C8:0 | 13 | rs110695408 | 68462615 | A | G |
| 200 | C8:0 | 13 | rs41631532 | 68468981 | G | A |
| 201 | C8:0 | 13 | rs133762197 | 68473203 | T | C |
| 202 | C8:0 | 13 | rs109215283 | 68487794 | A | G |
| 203 | C8:0 | 13 | rs109548237 | 68492293 | T | G |
| 204 | C8:0 | 13 | rs109802350 | 68503376 | A | G |
| 205 | C8:0 | 13 | rs133387349 | 68505109 | T | C |
| 206 | C8:0 | 13 | rs109762226 | 68506186 | A | G |
| 207 | C8:0 | 13 | rs136130202 | 68521184 | A | G |
| 208 | C8:0 | 13 | rs133603285 | 68521969 | T | C |
| 209 | C8:0 | 13 | rs134928364 | 68523290 | T | G |
| 210 | C8:0 | 13 | rs109812256 | 68524141 | A | G |
| 211 | C8:0 | 13 | rs110322216 | 68524730 | G | A |
| 212 | C8:0 | 13 | rs136586035 | 68905151 | A | G |
| 213 | C8:0 | 13 | rs109707307 | 68906269 | C | T |
| 214 | C8:0 | 13 | rs137634182 | 68911842 | T | C |
| 215 | C8:0 | 13 | rs134363250 | 68920327 | C | T |
| 216 | C8:0 | 13 | rs133185011 | 68925287 | C | T |
| 217 | C8:0 | 13 | rs109620329 | 68931367 | C | T |
| 218 | C6:0 | 13 | rs110840242 | 69070669 | G | T |
| | C8:0 | | | | G | T |
| 219 | C12:0 | 13 | rs109403285 | 73750114 | C | T |
| 220 | C6:0 | 13 | rs109085782 | 77948974 | G | T |
| | C8:0 | | | | G | T |
| 221 | C6:0 | 13 | rs42924695 | 77962539 | T | C |
| 222 | C6:0 | 13 | rs42924711 | 77971426 | A | G |
| 223 | C6:0 | 13 | rs41713942 | 77991304 | C | A |
| 224 | C10:0 | 13 | rs41700745 | 64639392 | C | T |
| | C12:0 | | | | C | T |
| | C14:0 | | | | C | T |
| | C6:0 | | | | C | T |
| | C8:0 | | | | C | T |
| | DNS | | | | C | T |
| 225 | C10:0 | 13 | rs41700742 | 64648620 | A | G |
| | C12:0 | | | | A | G |
| | C14:0 | | | | A | G |
| | C6:0 | | | | A | G |
| | C8:0 | | | | A | G |
| | DNS | | | | A | G |
| 226 | C10:0 | 13 | rs41700737 | 64655588 | A | G |
| | C12:0 | | | | A | G |
| | C14:0 | | | | A | G |
| | C6:0 | | | | A | G |
| | C8:0 | | | | A | G |
| | DNS | | | | A | G |
| 227 | C8:0 | 13 | rs41700736 | 64665113 | A | G |
| 228 | C8:0 | 13 | rs109284257 | 64675657 | T | G |
| 229 | C8:0 | 13 | rs43717522 | 64677620 | T | C |
| 230 | C8:0 | 13 | rs43717523 | 64679414 | G | A |
| 231 | C8:0 | 13 | rs43717484 | 64717871 | A | T |
| | DNS | | | | A | T |
| 232 | DNS | 13 | rs41631583 | 65280649 | G | A |
| 233 | C18:1cis-9 | 5 | rs136287575 | 93943798 | C | T |
| 234 | C18:1cis-9 | 5 | rs110489943 | 94901838 | C | A |
| 235 | C18:1cis-9 | 5 | rs136663774 | 94903186 | G | A |
| 236 | C18:1cis-9 | 5 | rs133770935 | 94908043 | C | A |
| 237 | C18:1cis-9 | 5 | rs135667197 | 94913073 | C | A |
| 238 | C18:1cis-9 | 5 | rs109696313 | 94984140 | T | C |
| 239 | C18:1cis-9 | 5 | rs137700249 | 95007471 | C | T |
| 240 | C18:1cis-9 | 5 | rs134037603 | 95025828 | A | G |
| 241 | C16:0 | 11 | rs109331017 | 102823112 | T | G |
| 242 | C18:1cis-9 | 11 | rs135586826 | 103293363 | G | A |
| | C16:0 | | | | A | G |
| | C4:0 | | | | A | G |
| 243 | C4:0 | 11 | rs110186753 | 103302351 | T | C |
| | C16:0 | | | | T | C |
| | C18:1cis-9 | | | | C | T |
| | C14:1cis-9 | | | | C | T |
| 244 | C4:0 | 11 | rs110143060 | 103304643 | T | C |
| | C16:0 | | | | T | C |
| | C18:1cis-9 | | | | C | T |
| | C14:1cis-9 | | | | C | T |
| 245 | C18:1cis-9 | 11 | rs109087963 | 103308330 | A | G |
| | C16:0 | | | | G | A |
| | C4:0 | | | | G | A |
| | C14:1cis-9 | | | | A | G |
| 246 | C4:0 | 11 | rs135786912 | 103317601 | G | T |
| | C16:0 | | | | G | T |
| | C18:1cis-9 | | | | T | G |
| | C14:1cis-9 | | | | T | G |
| 247 | C16:0 | 11 | rs136428773 | 103319019 | C | T |
| | C4:0 | | | | C | T |
| 248 | C16:0 | 11 | rs110755821 | 103321851 | C | T |
| 249 | C6:0 | 17 | rs137785016 | 48355536 | A | G |
| 250 | C6:0 | 17 | rs135583765 | 48361819 | C | T |
| 251 | C4:0 | 17 | rs41572286 | 48976925 | G | A |
| 252 | C4:0 | 17 | rs109188645 | 49580330 | C | T |
| 253 | C6:0 | 17 | rs135427193 | 50117287 | G | T |
| 254 | C8:0 | 17 | rs109783607 | 51076762 | A | G |
| | C6:0 | | | | A | G |
| 255 | C6:0 | 17 | rs109106552 | 51078591 | C | T |
| 256 | C6:0 | 17 | rs109169489 | 51097333 | T | G |
| 257 | C6:0 | 17 | rs137580316 | 51599696 | T | C |
| 258 | C6:0 | 17 | rs110872295 | 51668957 | C | T |
| | C8:0 | | | | C | T |
| 259 | C6:0 | 17 | rs137014685 | 51682489 | C | T |
| | C8:0 | | | | C | T |
| 260 | C6:0 | 17 | rs136965059 | 51768322 | A | G |
| 261 | C4:0 | 17 | rs110689162 | 52740162 | C | T |
| 262 | C4:0 | 17 | rs109629120 | 52743106 | C | T |
| 263 | C4:0 | 17 | rs110726640 | 52743970 | G | T |
| 264 | C4:0 | 17 | rs109533435 | 52745714 | C | A |
| 265 | C4:0 | 17 | rs110836032 | 52746731 | C | T |
| 266 | C4:0 | 17 | rs109989624 | 52747491 | G | A |
| 267 | C6:0 | 17 | rs137761118 | 52772547 | G | A |
| | C4:0 | | | | G | A |
| 268 | C8:0 | 17 | rs109371141 | 53866646 | A | G |
| 269 | C4:0 | 17 | rs133978310 | 53946879 | A | G |
| 270 | C8:0 | 17 | rs137653132 | 56174646 | T | C |
| 271 | C18:1cis-9 | 19 | rs109672704 | 31379078 | A | G |
| 272 | C18:1cis-9 | 19 | rs109327080 | 31382204 | T | C |
| 273 | C18:1cis-9 | 19 | rs109555691 | 31391100 | A | G |
| 274 | C18:1cis-9 | 19 | rs41908647 | 35648763 | C | A |
| 275 | C14:0 | 19 | rs29017737 | 36822248 | T | C |
| | C18:1cis-9 | | | | C | T |
| 276 | C12:0 | 19 | rs134057897 | 36832507 | C | T |
| | C14:0 | | | | C | T |
| | C18:1cis-9 | | | | T | C |
| 277 | C18:1cis-9 | 19 | rs109993655 | 36833660 | A | G |
| 278 | C18:1cis-9 | 19 | rs17870773 | 36834825 | C | T |
| 279 | C14:0 | 19 | rs110311467 | 37277118 | C | T |
| 280 | C14:0 | 19 | rs133472242 | 37278241 | C | T |
| | C18:1cis-9 | | | | T | C |
| 281 | C18:1cis-9 | 19 | rs110273711 | 37281135 | A | C |
| | C14:0 | | | | C | A |
| 282 | C18:1cis-9 | 19 | rs41913837 | 37281739 | A | G |
| 283 | C18:1cis-9 | 19 | rs133455441 | 37289888 | C | T |
| 284 | C14:0 | 19 | rs136512659 | 37294490 | C | T |
| 285 | C8:0 | 19 | rs41913893 | 37304511 | C | T |
| | C10:0 | | | | C | T |
| | C12:0 | | | | C | T |
| | C14:0 | | | | C | T |
| | C18:1cis-9 | | | | T | C |
| 286 | C8:0 | 19 | rs136626835 | 37324253 | T | G |
| | C10:0 | | | | T | G |
| | C12:0 | | | | T | G |
| | C14:0 | | | | T | G |
| 287 | C8:0 | 19 | rs41913877 | 37326298 | T | C |
| | C10:0 | | | | T | C |
| | C12:0 | | | | T | C |
| | C14:0 | | | | T | C |
| 288 | C8:0 | 19 | rs41576366 | 37396572 | T | G |
| | C10:0 | | | | T | G |
| | C12:0 | | | | T | G |
| | C14:0 | | | | T | G |
| 289 | C14:0 | 19 | rs41922131 | 51307828 | C | T |
| | C18:1cis-9 | | | | T | C |
| 290 | C18:1cis-9 | 19 | rs108973837 | 51312108 | G | A |
| | C14:0 | | | | A | G |
| | C12:0 | | | | A | G |
| 291 | C14:0 | 19 | rs41921159 | 51319695 | T | C |
| | C18:1cis-9 | | | | C | T |
| | C12:0 | | | | T | C |
| 292 | C18:1cis-9 | 19 | rs41921164 | 51320976 | G | A |
| | C14:0 | | | | A | G |
| | C12:0 | | | | A | G |
| 293 | C14:0 | 19 | rs41921166 | 51322878 | T | C |
| | C18:1cis-9 | | | | C | T |
| | C12:0 | | | | T | C |
| 294 | C14:0 | 19 | rs41921170 | 51323848 | G | T |
| | C18:1cis-9 | | | | T | G |
| | C12:0 | | | | G | T |
| 295 | C18:1cis-9 | 19 | rs41921171 | 51325151 | G | A |
| | C14:0 | | | | A | G |
| | C12:0 | | | | A | G |
| 296 | C18:1cis-9 | 19 | rs41921177 | 51326750 | G | A |
| | C14:0 | | | | A | G |
| | C12:0 | | | | A | G |
| 297 | C10:0 | 19 | rs136244801 | 51333432 | T | C |
| | C12:0 | | | | T | C |
| | C14:0 | | | | T | C |
| | C18:1cis-9 | | | | C | T |
| 298 | C18:1cis-9 | 19 | rs41921146 | 51341014 | G | A |
| | C14:0 | | | | A | G |
| | C12:0 | | | | A | G |
| | C10:0 | | | | A | G |
| 299 | C18:1cis-9 | 19 | rs41921143 | 51343311 | G | A |
| | C14:0 | | | | A | G |
| | C12:0 | | | | A | G |
| | C10:0 | | | | A | G |
| 300 | C18:1cis-9 | 19 | rs41921140 | 51349695 | G | A |
| | C14:0 | | | | A | G |
| | C12:0 | | | | A | G |
| | C10:0 | | | | A | G |
| 301 | C18:1cis-9 | 19 | rs41920007 | 51380689 | C | A |
| | C14:0 | | | | A | C |
| | C12:0 | | | | A | C |
| | C10:0 | | | | A | C |
| 302 | C8:0 | 19 | rs137372738 | 51386735 | C | T |
| | C10:0 | | | | C | T |
| | C12:0 | | | | C | T |
| | C14:0 | | | | C | T |
| | C18:1cis-9 | | | | T | C |
| 303 | C16:0 | 27 | rs110519353 | 36155097 | C | T |
| 304 | C16:0 | 27 | rs41585476 | 36208029 | A | G |
| 305 | C16:0 | 27 | rs109248310 | 36258043 | A | G |
| 306 | C16:0 | 27 | rs137660318 | 36259278 | T | C |
| 307 | C16:0 | 27 | rs133436102 | 36260026 | T | C |
| 308 | C16:0 | 27 | rs109632695 | 36263175 | A | G |
| 309 | C16:0 | 27 | rs109528207 | 36263939 | T | C |
| 310 | C16:0 | 27 | rs135357086 | 36265705 | C | T |
| 311 | C4:0 | 11 | rs110270048 | 103291134 | T | C |
| | C16:0 | | | | T | C |
| | C18:1cis-9 | | | | C | T |
| 312 | C4:0 | 11 | rs109333988 | 103291146 | C | G |
| | C16:0 | | | | C | G |
| | C18:1cis-9 | | | | G | C |
| 313 | C4:0 | 11 | rs136445488 | 103291159 | C | T |
| | C18:1cis-9 | | | | T | C |
| 314 | C4:0 | 11 | rs110051628 | 103291319 | A | G |
| | C16:0 | | | | A | G |
| | C14:1cis-9 | | | | G | A |
| | C18:1cis-9 | | | | G | A |
| 315 | C4:0 | 11 | rs381584633 | 103291361 | TA | T |
| | C16:0 | | | | TA | T |
| | C14:1cis-9 | | | | T | TA |
| | C18:1cis-9 | | | | T | TA |
| 316 | C4:0 | 11 | rs384433424 | 103291484 | GC | G |
| | C16:0 | | | | GC | G |
| | C14:1cis-9 | | | | G | GC |
| | C18:1cis-9 | | | | G | GC |
| 317 | C4:0 | 11 | rs208736858 | 103291675 | A | G |
| | C16:0 | | | | A | G |
| | C14:1cis-9 | | | | G | A |
| | C18:1cis-9 | | | | G | A |
| 318 | C4:0 | 11 | rs110769099 | 103291679 | T | C |
| | C16:0 | | | | T | C |
| | C14:1cis-9 | | | | C | T |
| | C18:1cis-9 | | | | C | T |
| 319 | C4:0 | 11 | rs209171393 | 103291891 | C | T |
| | C16:0 | | | | C | T |
| | C14:1cis-9 | | | | T | C |
| | C18:1cis-9 | | | | T | C |
| 320 | C4:0 | 11 | rs210927678 | 103291906 | A | T |
| | C16:0 | | | | A | T |
| | C14:1cis-9 | | | | T | A |
| | C18:1cis-9 | | | | T | A |
| 321 | C4:0 | 11 | rs110531467 | 103291944 | T | G |
| | C16:0 | | | | T | G |
| | C14:1cis-9 | | | | G | T |
| | C18:1cis-9 | | | | G | T |
| 322 | C4:0 | 11 | rs209455741 | 103292014 | T | G |
| | C16:0 | | | | T | G |
| | C14:1cis-9 | | | | G | T |
| | C18:1cis-9 | | | | G | T |
| 323 | C4:0 | 11 | rs110837274 | 103292067 | C | A |
| | C16:0 | | | | C | A |
| | C14:1cis-9 | | | | A | C |
| | C18:1cis-9 | | | | A | C |
| 324 | C4:0 | 11 | rs383493080 | 103292127 | A | G |
| | C16:0 | | | | A | G |
| | C14:1cis-9 | | | | G | A |
| | C18:1cis-9 | | | | G | A |
| 325 | C4:0 | 11 | rs377820370 | 103292307 | G | A |
| | C16:0 | | | | G | A |
| | C14:1cis-9 | | | | A | G |
| | C18:1cis-9 | | | | A | G |
| 326 | C4:0 | 11 | rs382086188 | 103292320 | A | G |
| | C16:0 | | | | A | G |
| | C14:1cis-9 | | | | G | A |
| | C18:1cis-9 | | | | G | A |
| 327 | C4:0 | 11 | rs383398415 | 103292402 | C | A |
| | C16:0 | | | | C | A |
| | C14:1cis-9 | | | | A | C |
| | C18:1cis-9 | | | | A | C |
| 328 | C4:0 | 11 | rs378684690 | 103292448 | G | T |
| | C16:0 | | | | G | T |
| | C14:1cis-9 | | | | T | G |
| | C18:1cis-9 | | | | T | G |
| 329 | C4:0 | 11 | rs381316841 | 103292468 | A | T |
| | C16:0 | | | | A | T |
| | C14:1cis-9 | | | | T | A |
| | C18:1cis-9 | | | | T | A |
| 330 | C4:0 | 11 | rs384539253 | 103292523 | C | T |
| | C16:0 | | | | C | T |
| | C14:1cis-9 | | | | T | C |
| | C18:1cis-9 | | | | T | C |
| 331 | C4:0 | 11 | rs379221045 | 103292572 | G | A |
| | C16:0 | | | | G | A |
| | C14:1cis-9 | | | | A | G |
| | C18:1cis-9 | | | | A | G |
| 332 | C4:0 | 11 | rs381050299 | 103292575 | C | T |
| | C16:0 | | | | C | T |
| | C14:1cis-9 | | | | T | C |
| | C18:1cis-9 | | | | T | C |
| 333 | C16:0 | 11 | rs384126999 | 103292628 | A | C |
| | C18:1cis-9 | | | | C | A |
| 334 | C4:0 | 11 | rs383134813 | 103292677 | C | G |
| | C16:0 | | | | C | G |
| | C14:1cis-9 | | | | G | C |
| | C18:1cis-9 | | | | G | C |
| 335 | C4:0 | 11 | rs517746215 | 103292741 | G | C |
| | C16:0 | | | | G | C |
| | C14:1cis-9 | | | | C | G |
| 336 | C4:0 | 11 | rs438581757 | 103292742 | G | A |
| | C16:0 | | | | G | A |
| | C14:1cis-9 | | | | A | G |
| 337 | C4:0 | 11 | rs458709486 | 103292747 | A | C |
| | C16:0 | | | | A | C |
| | C14:1cis-9 | | | | C | A |
| 338 | C4:0 | 11 | rs799698615 | 103292760 | T | C |
| | C16:0 | | | | T | C |
| | C14:1cis-9 | | | | C | T |
| 339 | C4:0 | 11 | rs797673592 | 103292762 | G | T |
| | C16:0 | | | | G | T |
| | C14:1cis-9 | | | | T | G |
| | C18:1cis-9 | | | | T | G |
| 340 | C4:0 | 11 | rs379863485 | 103292781 | T | C |
| | C16:0 | | | | T | C |
| | C14:1cis-9 | | | | C | T |
| | C18:1cis-9 | | | | C | T |
| 341 | C4:0 | 11 | rs382950378 | 103292802 | G | T |
| | C16:0 | | | | G | T |
| 342 | C4:0 | 11 | rs385886579 | 103292803 | A | G |
| | C16:0 | | | | A | G |
| 343 | C4:0 | 11 | rs380505424 | 103292935 | C | T |
| | C16:0 | | | | C | T |
| | C18:1cis-9 | | | | T | C |
| 344 | C16:0 | 11 | rs383532834 | 103292945 | A | G |
| | C18:1cis-9 | | | | G | A |
| 345 | C18:1cis-9 | 11 | rs385457481 | 103292970 | A | G |
| 346 | C4:0 | 11 | rs383579476 | 103293198 | G | T |
| | C16:0 | | | | G | T |
| | C14:1cis-9 | | | | T | G |
| | C18:1cis-9 | | | | T | G |
| 347 | C4:0 | 11 | rs109971913 | 103293370 | A | G |
| | C16:0 | | | | A | G |
| | C14:1cis-9 | | | | G | A |
| | C18:1cis-9 | | | | G | A |
| 348 | C4:0 | 11 | rs109539834 | 103293458 | T | C |
| | C16:0 | | | | T | C |
| | C14:1cis-9 | | | | C | T |
| | C18:1cis-9 | | | | C | T |
| 349 | C4:0 | 11 | rs109209173 | 103293485 | C | T |
| | C16:0 | | | | C | T |
| | C14:1cis-9 | | | | T | C |
| | C18:1cis-9 | | | | T | C |
| 350 | C4:0 | 11 | rs209767544 | 103293584 | C | T |
| | C16:0 | | | | C | T |
| | C14:1cis-9 | | | | T | C |
| | C18:1cis-9 | | | | T | C |
| 351 | C4:0 | 11 | rs207729763 | 103293600 | T | C |
| | C16:0 | | | | T | C |
| | C14:1cis-9 | | | | C | T |
| | C18:1cis-9 | | | | C | T |
| 352 | C4:0 | 11 | rs109454381 | 103293623 | A | T |
| | C16:0 | | | | A | T |
| | C14:1cis-9 | | | | T | A |
| | C18:1cis-9 | | | | T | A |
| 353 | C4:0 | 11 | rs109026837 | 103293813 | T | C |
| | C16:0 | | | | T | C |
| | C18:1cis-9 | | | | C | T |
| 354 | C4:0 | 11 | rs380721908 | 103293911 | A | G |
| | C16:0 | | | | A | G |
| | C14:1cis-9 | | | | G | A |
| | C18:1cis-9 | | | | G | A |
| 355 | C4:0 | 11 | rs110303032 | 103293921 | T | C |
| | C16:0 | | | | T | C |
| | C14:1cis-9 | | | | C | T |
| | C18:1cis-9 | | | | C | T |
| 356 | C4:0 | 11 | rs209767082 | 103293968 | A | G |
| | C16:0 | | | | A | G |
| | C14:1cis-9 | | | | G | A |
| | C18:1cis-9 | | | | G | A |
| 357 | C4:0 | 11 | rs109272848 | 103294139 | T | C |
| | C16:0 | | | | T | C |
| | C14:1cis-9 | | | | C | T |
| | C18:1cis-9 | | | | C | T |
| 358 | C4:0 | 11 | rs455411619 | 103294245 | G | GGT |
| | C16:0 | | | | G | GGT |
| | C14:1cis-9 | | | | GGT | G |
| | C18:1cis-9 | | | | GGT | G |
| 359 | C4:0 | 11 | rs383822350 | 103294259 | G | T |
| | C16:0 | | | | G | T |
| | C14:1cis-9 | | | | T | G |
| | C18:1cis-9 | | | | T | G |
| 360 | C4:0 | 11 | rs377841524 | 103294366 | A | G |
| | C16:0 | | | | A | G |
| | C14:1cis-9 | | | | G | A |
| | C18:1cis-9 | | | | G | A |
| 361 | C4:0 | 11 | rs109733270 | 103294403 | G | C |
| | C16:0 | | | | G | C |
| | C14:1cis-9 | | | | C | G |
| | C18:1cis-9 | | | | C | G |
| 362 | C4:0 | 11 | rs109531047 | 103294429 | A | G |
| | C16:0 | | | | A | G |
| | C14:1cis-9 | | | | G | A |
| | C18:1cis-9 | | | | G | A |
| 363 | C4:0 | 11 | rs110076614 | 103294434 | A | T |
| | C16:0 | | | | A | T |
| | C14:1cis-9 | | | | T | A |
| | C18:1cis-9 | | | | T | A |
| 364 | C4:0 | 11 | rs110372120 | 103294605 | A | T |
| | C16:0 | | | | A | T |
| | C18:1cis-9 | | | | T | A |
| 365 | C4:0 | 11 | rs110063984 | 103294607 | G | T |
| | C16:0 | | | | G | T |
| | C18:1cis-9 | | | | T | G |
| 366 | C4:0 | 11 | rs110388337 | 103294682 | C | A |
| | C16:0 | | | | C | A |
| | C14:1cis-9 | | | | A | C |
| | C18:1cis-9 | | | | A | C |
| 367 | C4:0 | 11 | rs207607053 | 103294772 | C | T |
| | C16:0 | | | | C | T |
| | C14:1cis-9 | | | | T | C |
| | C18:1cis-9 | | | | T | C |
| 368 | C4:0 | 11 | rs109361146 | 103294818 | T | G |
| | C16:0 | | | | T | G |
| | C14:1cis-9 | | | | G | T |
| | C18:1cis-9 | | | | G | T |
| 369 | C4:0 | 11 | rs110377516 | 103294930 | G | A |
| | C16:0 | | | | G | A |
| | C14:1cis-9 | | | | A | G |
| | C18:1cis-9 | | | | A | G |
| 370 | C4:0 | 11 | rs110143023 | 103295050 | C | G |
| | C16:0 | | | | C | G |
| | C14:1cis-9 | | | | G | C |
| | C18:1cis-9 | | | | G | C |
| 371 | C4:0 | 11 | rs109286526 | 103295211 | G | C |
| | C16:0 | | | | G | C |
| | C14:1cis-9 | | | | C | G |
| | C18:1cis-9 | | | | C | G |
| 372 | C4:0 | 11 | rs208026057 | 103295526 | T | C |
| | C16:0 | | | | T | C |
| | C14:1cis-9 | | | | C | T |
| | C18:1cis-9 | | | | C | T |
| 373 | C4:0 | 11 | rs110512392 | 103295907 | A | G |
| | C16:0 | | | | A | G |
| | C14:1cis-9 | | | | G | A |
| | C18:1cis-9 | | | | G | A |
| 374 | C4:0 | 11 | rs381989107 | 103296192 | C | A |
| | C16:0 | | | | C | A |
| | C14:1cis-9 | | | | A | C |
| | C18:1cis-9 | | | | A | C |
| 375 | C4:0 | 11 | rs385106761 | 103296261 | C | G |
| | C16:0 | | | | C | G |
| | C14:1cis-9 | | | | G | C |
| | C18:1cis-9 | | | | G | C |
| 376 | C4:0 | 11 | rs379692402 | 103296321 | C | T |
| | C16:0 | | | | C | T |
| | C14:1cis-9 | | | | T | C |
| | C18:1cis-9 | | | | T | C |
| 377 | C4:0 | 11 | rs383037149 | 103296337 | G | C |
| | C16:0 | | | | G | C |
| | C14:1cis-9 | | | | C | G |
| | C18:1cis-9 | | | | C | G |
| 378 | C4:0 | 11 | rs385328050 | 103296383 | C | T |
| | C16:0 | | | | C | T |
| | C14:1cis-9 | | | | T | C |
| | C18:1cis-9 | | | | T | C |
| 379 | C4:0 | 11 | rs380406578 | 103296877 | C | T |
| | C16:0 | | | | C | T |
| | C14:1cis-9 | | | | T | C |
| | C18:1cis-9 | | | | T | C |
| 380 | C4:0 | 11 | rs383436530 | 103296920 | C | T |
| | C16:0 | | | | C | T |
| | C14:1cis-9 | | | | T | C |
| | C18:1cis-9 | | | | T | C |
| 381 | C4:0 | 11 | rs109628546 | 103297341 | G | A |
| | C16:0 | | | | G | A |
| | C14:1cis-9 | | | | A | G |
| | C18:1cis-9 | | | | A | G |
| 382 | C4:0 | 11 | rs208280659 | 103297793 | T | C |
| | C16:0 | | | | T | C |
| | C14:1cis-9 | | | | C | T |
| | C18:1cis-9 | | | | C | T |
| 383 | C4:0 | 11 | rs109817665 | 103297968 | C | T |
| | C16:0 | | | | C | T |
| | C14:1cis-9 | | | | T | C |
| | C18:1cis-9 | | | | T | C |
| 384 | C4:0 | 11 | rs211084229 | 103298098 | C | T |
| | C16:0 | | | | C | T |
| | C14:1cis-9 | | | | T | C |
| | C18:1cis-9 | | | | T | C |
| 385 | C4:0 | 11 | rs208564616 | 103298237 | C | G |
| | C16:0 | | | | C | G |
| | C14:1cis-9 | | | | G | C |
| | C18:1cis-9 | | | | G | C |
| 386 | C4:0 | 11 | rs211132416 | 103298241 | A | G |
| | C16:0 | | | | A | G |
| | C14:1cis-9 | | | | G | A |
| | C18:1cis-9 | | | | G | A |
| 387 | C4:0 | 11 | rs109990597 | 103298254 | A | G |
| | C16:0 | | | | A | G |
| | C14:1cis-9 | | | | G | A |
| | C18:1cis-9 | | | | G | A |
| 388 | C4:0 | 11 | rs110281106 | 103298257 | C | T |
| | C16:0 | | | | C | T |
| | C14:1cis-9 | | | | T | C |
| | C18:1cis-9 | | | | T | C |
| 389 | C4:0 | 11 | rs109955364 | 103298374 | T | C |
| | C16:0 | | | | T | C |
| | C14:1cis-9 | | | | C | T |
| | C18:1cis-9 | | | | C | T |
| 390 | C4:0 | 11 | rs109046993 | 103298385 | T | C |
| | C16:0 | | | | T | C |
| | C14:1cis-9 | | | | C | T |
| | C18:1cis-9 | | | | C | T |
| 391 | C4:0 | 11 | rs109166248 | 103298431 | A | T |
| | C16:0 | | | | A | T |
| | C14:1cis-9 | | | | T | A |
| | C18:1cis-9 | | | | T | A |
| 392 | C4:0 | 11 | rs110661775 | 103298458 | T | G |
| | C16:0 | | | | T | G |
| | C14:1cis-9 | | | | G | T |
| | C18:1cis-9 | | | | G | T |
| 393 | C4:0 | 11 | rs110276402 | 103298698 | T | A |
| | C16:0 | | | | T | A |
| | C18:1cis-9 | | | | A | T |
| 394 | C4:0 | 11 | rs109008083 | 103298745 | C | T |
| | C16:0 | | | | C | T |
| | C14:1cis-9 | | | | T | C |
| | C18:1cis-9 | | | | T | C |
| 395 | C4:0 | 11 | rs110703191 | 103299004 | T | C |
| | C16:0 | | | | T | C |
| | C14:1cis-9 | | | | C | T |
| | C18:1cis-9 | | | | C | T |
| 396 | C4:0 | 11 | rs209121343 | 103299184 | A | G |
| | C16:0 | | | | A | G |
| | C14:1cis-9 | | | | G | A |
| | C18:1cis-9 | | | | G | A |
| 397 | C4:0 | 11 | rs110563549 | 103299272 | G | A |
| | C16:0 | | | | G | A |
| | C18:1cis-9 | | | | A | G |
| 398 | C4:0 | 11 | rs109554394 | 103299275 | G | C |
| | C16:0 | | | | G | C |
| | C14:1cis-9 | | | | C | G |
| | C18:1cis-9 | | | | C | G |
| 399 | C4:0 | 11 | rs110860386 | 103299387 | G | A |
| | C16:0 | | | | G | A |
| | C14:1cis-9 | | | | A | G |
| | C18:1cis-9 | | | | A | G |
| 400 | C4:0 | 11 | rs110802466 | 103299453 | A | G |
| | C16:0 | | | | A | G |
| | C14:1cis-9 | | | | G | A |
| | C18:1cis-9 | | | | G | A |
| 401 | C4:0 | 11 | rs110933198 | 103299655 | C | T |
| | C16:0 | | | | C | T |
| | C14:1cis-9 | | | | T | C |
| | C18:1cis-9 | | | | T | C |
| 402 | C4:0 | 11 | rs109688459 | 103299687 | T | C |
| | C16:0 | | | | T | C |
| | C14:1cis-9 | | | | C | T |
| | C18:1cis-9 | | | | C | T |
| 403 | C4:0 | 11 | rs110659098 | 103299696 | C | T |
| | C16:0 | | | | C | T |
| | C14:1cis-9 | | | | T | C |
| | C18:1cis-9 | | | | T | C |
| 404 | C4:0 | 11 | rs109733140 | 103299747 | T | C |
| | C16:0 | | | | T | C |
| | C14:1cis-9 | | | | C | T |
| | C18:1cis-9 | | | | C | T |
| 405 | C4:0 | 11 | rs109509741 | 103299801 | C | T |
| | C16:0 | | | | C | T |
| | C14:1cis-9 | | | | T | C |
| | C18:1cis-9 | | | | T | C |
| 406 | C4:0 | 11 | rs461776512 | 103299807 | TG | T |
| | C16:0 | | | | TG | T |
| | C14:1cis-9 | | | | T | TG |
| | C18:1cis-9 | | | | T | TG |
| 407 | C4:0 | 11 | rs110710904 | 103299858 | C | G |
| | C16:0 | | | | C | G |
| | C14:1cis-9 | | | | G | C |
| | C18:1cis-9 | | | | G | C |
| 408 | C4:0 | 11 | rs209693172 | 103299916 | C | G |
| | C16:0 | | | | C | G |
| | C14:1cis-9 | | | | G | C |
| | C18:1cis-9 | | | | G | C |
| 409 | C4:0 | 11 | rs211534131 | 103299937 | C | T |
| | C16:0 | | | | C | T |
| | C14:1cis-9 | | | | T | C |
| | C18:1cis-9 | | | | T | C |
| 410 | C4:0 | 11 | rs208570651 | 103299941 | C | T |
| | C16:0 | | | | C | T |
| | C14:1cis-9 | | | | T | C |
| | C18:1cis-9 | | | | T | C |
| 411 | C4:0 | 11 | rs110387275 | 103300020 | T | C |
| | C16:0 | | | | T | C |
| | C18:1cis-9 | | | | C | T |
| 412 | C4:0 | 11 | rs109843613 | 103300067 | C | T |
| | C16:0 | | | | C | T |
| | C14:1cis-9 | | | | T | C |
| | C18:1cis-9 | | | | T | C |
| 413 | C4:0 | 11 | rs110750786 | 103300078 | G | T |
| | C16:0 | | | | G | T |
| | C14:1cis-9 | | | | T | G |
| | C18:1cis-9 | | | | T | G |
| 414 | C4:0 | 11 | rs110888023 | 103300244 | A | G |
| | C16:0 | | | | A | G |
| | C14:1cis-9 | | | | G | A |
| | C18:1cis-9 | | | | G | A |
| 415 | C4:0 | 11 | rs110053249 | 103300324 | A | G |
| | C16:0 | | | | A | G |
| | C14:1cis-9 | | | | G | A |
| | C18:1cis-9 | | | | G | A |
| 416 | C4:0 | 11 | rs109982707 | 103300548 | G | A |
| | C16:0 | | | | G | A |
| | C14:1cis-9 | | | | A | G |
| | C18:1cis-9 | | | | A | G |
| 417 | C4:0 | 11 | rs109105837 | 103300608 | C | A |
| | C16:0 | | | | C | A |
| | C14:1cis-9 | | | | A | C |
| | C18:1cis-9 | | | | A | C |
| 418 | C4:0 | 11 | rs110024463 | 103300672 | G | C |
| | C16:0 | | | | G | C |
| | C14:1cis-9 | | | | C | G |
| | C18:1cis-9 | | | | C | G |
| 419 | C4:0 | 11 | rs109837926 | 103300697 | A | C |
| | C16:0 | | | | A | C |
| | C14:1cis-9 | | | | C | A |
| | C18:1cis-9 | | | | C | A |
| 420 | C4:0 | 11 | rs110920335 | 103300718 | C | T |
| | C16:0 | | | | C | T |
| | C14:1cis-9 | | | | T | C |
| | C18:1cis-9 | | | | T | C |
| 421 | C4:0 | 11 | rs110335984 | 103300719 | A | G |
| | C16:0 | | | | A | G |
| | C14:1cis-9 | | | | G | A |
| | C18:1cis-9 | | | | G | A |
| 422 | C4:0 | 11 | rs109897196 | 103300725 | G | C |
| | C16:0 | | | | G | C |
| | C14:1cis-9 | | | | C | G |
| | C18:1cis-9 | | | | C | G |
| 423 | C4:0 | 11 | rs110766819 | 103300899 | G | A |
| | C16:0 | | | | G | A |
| | C14:1cis-9 | | | | A | G |
| | C18:1cis-9 | | | | A | G |
| 424 | C4:0 | 11 | rs41255675 | 103301002 | C | G |
| | C16:0 | | | | C | G |
| | C14:1cis-9 | | | | G | C |
| | C18:1cis-9 | | | | G | C |
| 425 | C4:0 | 11 | rs41255676 | 103301030 | T | C |
| | C16:0 | | | | T | C |
| | C14:1cis-9 | | | | C | T |
| | C18:1cis-9 | | | | C | T |
| 426 | C4:0 | 11 | rs41255677 | 103301063 | C | A |
| | C16:0 | | | | C | A |
| | C14:1cis-9 | | | | A | C |
| | C18:1cis-9 | | | | A | C |
| 427 | C4:0 | 11 | rs41255678 | 103301202 | G | C |
| | C16:0 | | | | G | C |
| | C14:1cis-9 | | | | C | G |
| | C18:1cis-9 | | | | C | G |
| 428 | C4:0 | 11 | rs41255679 | 103301229 | G | C |
| | C16:0 | | | | G | C |
| | C14:1cis-9 | | | | C | G |
| | C18:1cis-9 | | | | C | G |
| 429 | C4:0 | 11 | rs41255682 | 103301443 | T | C |
| | C16:0 | | | | T | C |
| | C14:1cis-9 | | | | C | T |
| | C18:1cis-9 | | | | C | T |
| 430 | C4:0 | 11 | rs41255683 | 103301455 | C | G |
| | C16:0 | | | | C | G |
| | C14:1cis-9 | | | | G | C |
| | C18:1cis-9 | | | | G | C |
| 431 | C4:0 | 11 | rs41255684 | 103301561 | C | G |
| | C16:0 | | | | C | G |
| | C14:1cis-9 | | | | G | C |
| | C18:1cis-9 | | | | G | C |
| 432 | C4:0 | 11 | rs41255685 | 103301690 | T | C |
| | C16:0 | | | | T | C |
| | C14:1cis-9 | | | | C | T |
| | C18:1cis-9 | | | | C | T |
| 433 | C4:0 | 11 | rs41255686 | 103301694 | G | A |
| | C16:0 | | | | G | A |
| | C14:1cis-9 | | | | A | G |
| | C18:1cis-9 | | | | A | G |
| 434 | C4:0 | 11 | rs41255687 | 103301736 | C | T |
| | C16:0 | | | | C | T |
| | C14:1cis-9 | | | | T | C |
| | C18:1cis-9 | | | | T | C |
| 435 | C4:0 | 11 | rs109116595 | 103301757 | C | T |
| | C16:0 | | | | C | T |
| | C14:1cis-9 | | | | T | C |
| | C18:1cis-9 | | | | T | C |
| 436 | C4:0 | 11 | rs110788821 | 103301805 | G | C |
| | C16:0 | | | | G | C |
| | C14:1cis-9 | | | | C | G |
| | C18:1cis-9 | | | | C | G |
| 437 | C4:0 | 11 | rs109907194 | 103301982 | A | G |
| | C16:0 | | | | A | G |
| | C14:1cis-9 | | | | G | A |
| | C18:1cis-9 | | | | G | A |
| 438 | C4:0 | 11 | rs110874271 | 103302129 | G | A |
| | C16:0 | | | | G | A |
| | C14:1cis-9 | | | | A | G |
| | C18:1cis-9 | | | | A | G |
| 439 | C4:0 | 11 | rs109947864 | 103302272 | C | T |
| | C16:0 | | | | C | T |
| | C14:1cis-9 | | | | T | C |
| | C18:1cis-9 | | | | T | C |
| 440 | C4:0 | 11 | rs109755032 | 103302429 | G | C |
| | C16:0 | | | | G | C |
| | C14:1cis-9 | | | | C | G |
| | C18:1cis-9 | | | | C | G |
| 441 | C4:0 | 11 | rs383883089 | 103302617 | C | CA |
| | C16:0 | | | | C | CA |
| | C14:1cis-9 | | | | CA | C |
| | C18:1cis-9 | | | | CA | C |
| 442 | C4:0 | 11 | rs110537205 | 103302766 | T | C |
| | C16:0 | | | | T | C |
| | C14:1cis-9 | | | | C | T |
| | C18:1cis-9 | | | | C | T |
| 443 | C4:0 | 11 | rs43691047 | 103303242 | G | A |
| | C16:0 | | | | G | A |
| | C14:1cis-9 | | | | A | G |
| | C18:1cis-9 | | | | A | G |
| 444 | C4:0 | 11 | rs43691048 | 103303327 | G | A |
| | C16:0 | | | | G | A |
| | C14:1cis-9 | | | | A | G |
| | C18:1cis-9 | | | | A | G |
| 445 | C4:0 | 11 | rs43691049 | 103303343 | T | C |
| | C16:0 | | | | T | C |
| | C14:1cis-9 | | | | C | T |
| | C18:1cis-9 | | | | C | T |
| 446 | C4:0 | 11 | rs110066229 | 103303475 | A | G |
| | C16:0 | | | | A | G |
| | C14:1cis-9 | | | | G | A |
| | C18:1cis-9 | | | | G | A |
| 447 | C4:0 | 11 | rs110886675 | 103303619 | A | G |
| | C16:0 | | | | A | G |
| | C14:1cis-9 | | | | G | A |
| | C18:1cis-9 | | | | G | A |
| 448 | C4:0 | 11 | rs110205058 | 103303620 | G | A |
| | C16:0 | | | | G | A |
| | C14:1cis-9 | | | | A | G |
| | C18:1cis-9 | | | | A | G |
| 449 | C4:0 | 11 | rs110962910 | 103303700 | C | T |
| | C16:0 | | | | C | T |
| | C14:1cis-9 | | | | T | C |
| | C18:1cis-9 | | | | T | C |
| 450 | C4:0 | 11 | rs109498796 | 103303701 | A | G |
| | C16:0 | | | | A | G |
| | C14:1cis-9 | | | | G | A |
| | C18:1cis-9 | | | | G | A |
| 451 | C4:0 | 11 | rs109863113 | 103303792 | T | C |
| | C16:0 | | | | T | C |
| | C14:1cis-9 | | | | C | T |
| | C18:1cis-9 | | | | C | T |
| 452 | C4:0 | 11 | rs382320425 | 103303839 | T | TA |
| | C16:0 | | | | T | TA |
| | C14:1cis-9 | | | | TA | T |
| | C18:1cis-9 | | | | TA | T |
| 453 | C4:0 | 11 | rs211153297 | 103304094 | A | G |
| | C16:0 | | | | A | G |
| | C14:1cis-9 | | | | G | A |
| | C18:1cis-9 | | | | G | A |
| 454 | C4:0 | 11 | rs208613318 | 103304197 | G | A |
| | C16:0 | | | | G | A |
| | C14:1cis-9 | | | | A | G |
| | C18:1cis-9 | | | | A | G |
| 455 | C4:0 | 11 | rs210362322 | 103304235 | T | C |
| | C16:0 | | | | T | C |
| | C14:1cis-9 | | | | C | T |
| | C18:1cis-9 | | | | C | T |
| 456 | C4:0 | 11 | rs110941132 | 103304468 | G | A |
| | C16:0 | | | | G | A |
| | C14:1cis-9 | | | | A | G |
| | C18:1cis-9 | | | | A | G |
| 457 | C4:0 | 11 | rs110684335 | 103304476 | A | G |
| | C16:0 | | | | A | G |
| | C14:1cis-9 | | | | G | A |
| | C18:1cis-9 | | | | G | A |
| 458 | C4:0 | 11 | rs110992345 | 103304509 | C | T |
| | C16:0 | | | | C | T |
| | C14:1cis-9 | | | | T | C |
| | C18:1cis-9 | | | | T | C |
| 459 | C4:0 | 11 | rs110688769 | 103304510 | A | G |
| | C16:0 | | | | A | G |
| | C14:1cis-9 | | | | G | A |
| | C18:1cis-9 | | | | G | A |
| 460 | C4:0 | 11 | rs109890044 | 103304593 | C | T |
| | C16:0 | | | | C | T |
| | C14:1cis-9 | | | | T | C |
| | C18:1cis-9 | | | | T | C |
| 461 | C4:0 | 11 | rs109990218 | 103304656 | A | T |
| | C16:0 | | | | A | T |
| | C14:1cis-9 | | | | T | A |
| | C18:1cis-9 | | | | T | A |
| 462 | C4:0 | 11 | rs110641366 | 103304668 | C | T |
| | C16:0 | | | | C | T |
| | C14:1cis-9 | | | | T | C |
| | C18:1cis-9 | | | | T | C |
| 463 | C4:0 | 11 | rs109625649 | 103304757 | T | C |
| | C16:0 | | | | T | C |
| | C14:1cis-9 | | | | C | T |
| | C18:1cis-9 | | | | C | T |
| 464 | C4:0 | 11 | rs137003236 | 103304944 | T | C |
| | C16:0 | | | | T | C |
| | C14:1cis-9 | | | | C | T |
| | C18:1cis-9 | | | | C | T |
| 465 | C4:0 | 11 | rs207607418 | 103305230 | T | G |
| | C16:0 | | | | T | G |
| | C14:1cis-9 | | | | G | T |
| | C18:1cis-9 | | | | G | T |
| 466 | C4:0 | 11 | rs109021803 | 103305742 | A | G |
| | C16:0 | | | | A | G |
| | C14:1cis-9 | | | | G | A |
| | C18:1cis-9 | | | | G | A |
| 467 | C4:0 | 11 | rs110480949 | 103305744 | C | T |
| | C16:0 | | | | C | T |
| | C14:1cis-9 | | | | T | C |
| | C18:1cis-9 | | | | T | C |
| 468 | C4:0 | 11 | rs110405716 | 103306167 | G | A |
| | C16:0 | | | | G | A |
| | C14:1cis-9 | | | | A | G |
| | C18:1cis-9 | | | | A | G |
| 469 | C4:0 | 11 | rs383482709 | 103307124 | G | A |
| | C16:0 | | | | G | A |
| | C14:1cis-9 | | | | A | G |
| | C18:1cis-9 | | | | A | G |
| 470 | C4:0 | 11 | rs110935748 | 103307330 | G | A |
| | C16:0 | | | | G | A |
| | C14:1cis-9 | | | | A | G |
| | C18:1cis-9 | | | | A | G |
| 471 | C4:0 | 11 | rs110328877 | 103308459 | A | G |
| | C16:0 | | | | A | G |
| | C14:1cis-9 | | | | G | A |
| | C18:1cis-9 | | | | G | A |
| 472 | C4:0 | 11 | rs110351291 | 103312638 | G | A |
| | C16:0 | | | | G | A |
| | C14:1cis-9 | | | | A | G |
| | C18:1cis-9 | | | | A | G |
| 473 | C4:0 | 11 | rs210855396 | 103313920 | C | A |
| | C16:0 | | | | C | A |
| | C14:1cis-9 | | | | A | C |
| | C18:1cis-9 | | | | A | C |
| 474 | C4:0 | 11 | rs109880410 | 103315024 | G | A |
| | C16:0 | | | | G | A |
| | C14:1cis-9 | | | | A | G |
| | C18:1cis-9 | | | | A | G |
| 475 | C4:0 | 11 | rs133607453 | 103318190 | G | A |
| | C16:0 | | | | G | A |
| | C14:1cis-9 | | | | A | G |
| | C18:1cis-9 | | | | A | G |
| 476 | C4:0 | 11 | rs110513870 | 103321477 | C | T |
| 477 | C4:0 | 11 | rs210419726 | 103321929 | T | G |
| | C16:0 | | | | T | G |
| | C14:1cis-9 | | | | G | T |
| | C18:1cis-9 | | | | G | T |
| 478 | C4:0 | 11 | rs470960381 | 103321961 | T | TA |
| | C16:0 | | | | T | TA |
| | C14:1cis-9 | | | | TA | T |
| | C18:1cis-9 | | | | TA | T |
| 479 | C4:0 | 11 | rs109891490 | 103322072 | G | A |
| | C16:0 | | | | G | A |
| | C14:1cis-9 | | | | A | G |
| | C18:1cis-9 | | | | A | G |
| 480 | C4:0 | 11 | rs209097885 | 103322398 | G | A |
| | C16:0 | | | | G | A |
| | C14:1cis-9 | | | | A | G |
| | C18:1cis-9 | | | | A | G |
| 481 | C4:0 | 11 | rs110788899 | 103322890 | T | C |
| | C16:0 | | | | T | C |
| | C14:1cis-9 | | | | C | T |
| | C18:1cis-9 | | | | C | T |
| 482 | C4:0 | 11 | rs465985450 | 103323672 | A | AG |
| 483 | C14:0 | 13 | | 63086920 | A | C |
| 484 | C14:0 | 13 | | 63086927 | G | A |
| | C18:1cis-9 | | | | A | G |
| 485 | C14:0 | 13 | | 63086938 | G | A |
| | C18:1cis-9 | | | | A | G |
| 486 | C14:0 | 13 | | 63086943 | C | T |
| | C18:1cis-9 | | | | T | C |
| 487 | C12:0 | 13 | | 63086946 | G | A |
| | C14:0 | | | | G | A |
| | C18:1cis-9 | | | | A | G |
| 488 | C12:0 | 13 | | 63086958 | T | C |
| | C14:0 | | | | T | C |
| | C18:1cis-9 | | | | C | T |
| 489 | C12:0 | 13 | rs469941426 | 63086971 | C | T |
| | C14:0 | | | | C | T |
| | C18:1cis-9 | | | | T | C |
| 490 | C10:0 | 13 | | 63086974 | C | A |
| | C12:0 | | | | C | A |
| | C14:0 | | | | C | A |
| | C18:1cis-9 | | | | A | C |
| 491 | C10:0 | 13 | | 63086976 | C | T |
| | C12:0 | | | | C | T |
| | C14:0 | | | | C | T |
| | C18:1cis-9 | | | | T | C |
| 492 | C10:0 | 13 | | 63086979 | T | A |
| | C12:0 | | | | T | A |
| | C14:0 | | | | T | A |
| | C18:1cis-9 | | | | A | T |
| 493 | C10:0 | 13 | | 63086983 | C | A |
| | C12:0 | | | | C | A |
| | C14:0 | | | | C | A |
| | C18:1cis-9 | | | | A | C |
| 494 | C10:0 | 13 | | 63086994 | T | A |
| | C12:0 | | | | T | A |
| | C14:0 | | | | T | A |
| | C18:1cis-9 | | | | A | T |
| 495 | C6:0 | 13 | rs450388115 | 63095347 | C | T |
| | C8:0 | | | | C | T |
| | C12:0 | | | | C | T |
| 496 | C8:0 | 13 | rs460746052 | 63140316 | A | C |
| 497 | C12:0 | 13 | rs134471111 | 63228180 | A | G |
| | C14:0 | | | | A | G |
| | C18:1cis-9 | | | | G | A |
| 498 | C8:0 | 13 | rs384099886 | 63266009 | T | C |
| 499 | C6:0 | 13 | rs43711793 | 63270097 | A | T |
| | C8:0 | | | | A | T |
| 500 | C8:0 | 13 | rs384853631 | 63302895 | C | T |
| 501 | C6:0 | 13 | rs466662937 | 63507251 | G | A |
| | C8:0 | | | | G | A |
| | C10:0 | | | | G | A |
| | C12:0 | | | | G | A |
| | C14:0 | | | | G | A |
| 502 | C8:0 | 13 | rs476559105 | 63509737 | C | G |
| | C10:0 | | | | C | G |
| | C12:0 | | | | C | G |
| | C14:0 | | | | C | G |
| 503 | C6:0 | 13 | | 63521011 | G | A |
| | C8:0 | | | | G | A |
| | C10:0 | | | | G | A |
| | C12:0 | | | | G | A |
| | C14:0 | | | | G | A |
| 504 | C6:0 | 13 | | 63524193 | T | C |
| | C8:0 | | | | T | C |
| | C10:0 | | | | T | C |
| | C12:0 | | | | T | C |
| | C14:0 | | | | T | C |
| 505 | C6:0 | 13 | | 63525254 | G | GA |
| | C8:0 | | | | G | GA |
| | C10:0 | | | | G | GA |
| | C12:0 | | | | G | GA |
| | C14:0 | | | | G | GA |
| 506 | C6:0 | 13 | rs440895334 | 63525614 | G | A |
| | C8:0 | | | | G | A |
| | C10:0 | | | | G | A |
| | C12:0 | | | | G | A |
| | C14:0 | | | | G | A |
| 507 | C6:0 | 13 | rs446522735 | 63544624 | C | T |
| | C8:0 | | | | C | T |
| | C10:0 | | | | C | T |
| | C12:0 | | | | C | T |
| | C14:0 | | | | C | T |
| 508 | C6:0 | 13 | rs379551233 | 63572279 | G | A |
| | C8:0 | | | | G | A |
| | C10:0 | | | | G | A |
| | C12:0 | | | | G | A |
| | C14:0 | | | | G | A |
| 509 | C6:0 | 13 | rs382779775 | 63572280 | T | G |
| | C8:0 | | | | T | G |
| | C10:0 | | | | T | G |
| | C12:0 | | | | T | G |
| | C14:0 | | | | T | G |
| 510 | C6:0 | 13 | rs380845667 | 63617034 | G | A |
| | C8:0 | | | | G | A |
| | C10:0 | | | | G | A |
| | C12:0 | | | | G | A |
| | C14:0 | | | | G | A |
| 511 | C6:0 | 13 | rs471902607 | 63660381 | C | T |
| | C8:0 | | | | C | T |
| | C10:0 | | | | C | T |
| | C12:0 | | | | C | T |
| | C14:0 | | | | C | T |
| 512 | C6:0 | 13 | rs385104196 | 63728651 | G | A |
| | C8:0 | | | | G | A |
| | C10:0 | | | | G | A |
| | C12:0 | | | | G | A |
| | C14:0 | | | | G | A |
| 513 | C6:0 | 13 | rs378811160 | 63731561 | C | T |
| | C8:0 | | | | C | T |
| | C10:0 | | | | C | T |
| | C12:0 | | | | C | T |
| | C14:0 | | | | C | T |
| 514 | C6:0 | 13 | rs378303170 | 63734337 | C | T |
| | C8:0 | | | | C | T |
| | C10:0 | | | | C | T |
| | C12:0 | | | | C | T |
| | C14:0 | | | | C | T |
| 515 | C8:0 | 13 | rs454344378 | 63734389 | A | G |
| | C12:0 | | | | A | G |
| 516 | C8:0 | 13 | | 63735056 | GAGATGAG | GAGATGA |
| | | | | | TGCAATTG | GTGCAAT |
| | | | | | TGCA | TGTGCG |
| 517 | C6:0 | 13 | rs379649986 | 63741818 | T | C |
| | C8:0 | | | | T | C |
| | C10:0 | | | | T | C |
| | C12:0 | | | | T | C |
| | C14:0 | | | | T | C |
| 518 | C6:0 | 13 | rs380223633 | 63742135 | C | A |
| | C8:0 | | | | C | A |
| | C10:0 | | | | C | A |
| | C12:0 | | | | C | A |
| | C14:0 | | | | C | A |
| 519 | C6:0 | 13 | rs480865010 | 63742388 | T | G |
| | C8:0 | | | | T | G |
| | C10:0 | | | | T | G |
| | C12:0 | | | | T | G |
| | C14:0 | | | | T | G |
| 520 | C6:0 | 13 | rs380980005 | 63743062 | T | G |
| | C8:0 | | | | T | G |
| | C10:0 | | | | T | G |
| | C12:0 | | | | T | G |
| | C14:0 | | | | T | G |
| 521 | C6:0 | 13 | rs473249925 | 63743159 | G | A |
| | C8:0 | | | | G | A |
| | C10:0 | | | | G | A |
| | C12:0 | | | | G | A |
| | C14:0 | | | | G | A |
| 522 | C6:0 | 13 | rs441218700 | 63743195 | C | A |
| | C8:0 | | | | C | A |
| | C10:0 | | | | C | A |
| | C12:0 | | | | C | A |
| | C14:0 | | | | C | A |
| 523 | C6:0 | 13 | rs383480158 | 63743346 | T | G |
| | C8:0 | | | | T | G |
| | C10:0 | | | | T | G |
| | C12:0 | | | | T | G |
| | C14:0 | | | | T | G |
| 524 | C6:0 | 13 | rs377841389 | 63743596 | G | C |
| | C8:0 | | | | G | C |
| | C10:0 | | | | G | C |
| | C12:0 | | | | G | C |
| | C14:0 | | | | G | C |
| 525 | C6:0 | 13 | rs381415308 | 63747026 | T | C |
| | C8:0 | | | | T | C |
| | C10:0 | | | | T | C |
| | C12:0 | | | | T | C |
| | C14:0 | | | | T | C |
| 526 | C6:0 | 13 | rs380484412 | 63747747 | C | T |
| | C8:0 | | | | C | T |
| | C10:0 | | | | C | T |
| | C12:0 | | | | C | T |
| | C14:0 | | | | C | T |
| 527 | C6:0 | 13 | rs377908725 | 63753096 | C | T |
| | C8:0 | | | | C | T |
| | C10:0 | | | | C | T |
| | C12:0 | | | | C | T |
| | C14:0 | | | | C | T |
| 528 | C6:0 | 13 | rs464634573 | 63761735 | C | T |
| | C8:0 | | | | C | T |
| | C10:0 | | | | C | T |
| | C12:0 | | | | C | T |
| | C14:0 | | | | C | T |
| 529 | C6:0 | 13 | rs441127270 | 63765767 | C | T |
| | C8:0 | | | | C | T |
| | C10:0 | | | | C | T |
| | C12:0 | | | | C | T |
| | C14:0 | | | | C | T |
| 530 | C6:0 | 13 | rs720136600 | 63770658 | G | A |
| | C8:0 | | | | G | A |
| | C10:0 | | | | G | A |
| | C12:0 | | | | G | A |
| | C14:0 | | | | G | A |
| 531 | C6:0 | 13 | rs719865499 | 63773948 | G | A |
| | C8:0 | | | | G | A |
| | C10:0 | | | | G | A |
| | C12:0 | | | | G | A |
| | C14:0 | | | | G | A |
| 532 | C6:0 | 13 | rs475450155 | 63792022 | G | A |
| | C8:0 | | | | G | A |
| | C10:0 | | | | G | A |
| | C12:0 | | | | G | A |
| | C14:0 | | | | G | A |
| 533 | C6:0 | 13 | rs384902852 | 63792952 | C | T |
| | C8:0 | | | | C | T |
| | C10:0 | | | | C | T |
| | C12:0 | | | | C | T |
| | C14:0 | | | | C | T |
| 534 | C6:0 | 13 | rs382787142 | 63804097 | G | A |
| | C8:0 | | | | G | A |
| | C10:0 | | | | G | A |
| | C12:0 | | | | G | A |
| | C14:0 | | | | G | A |
| 535 | C6:0 | 13 | rs467176730 | 63808494 | G | T |
| | C8:0 | | | | G | T |
| | C10:0 | | | | G | T |
| | C12:0 | | | | G | T |
| | C14:0 | | | | G | T |
| 536 | C6:0 | 13 | rs378623574 | 63810159 | C | T |
| | C8:0 | | | | C | T |
| | C10:0 | | | | C | T |
| | C12:0 | | | | C | T |
| | C14:0 | | | | C | T |
| 537 | C6:0 | 13 | rs518047916 | 63816410 | A | T |
| | C8:0 | | | | A | T |
| | C10:0 | | | | A | T |
| | C12:0 | | | | A | T |
| | C14:0 | | | | A | T |
| 538 | C6:0 | 13 | | 63860287 | C | G |
| | C8:0 | | | | C | G |
| | C10:0 | | | | C | G |
| | C12:0 | | | | C | G |
| | C14:0 | | | | C | G |
| 539 | C6:0 | 13 | rs380118486 | 63882786 | G | T |
| | C8:0 | | | | G | T |
| | C10:0 | | | | G | T |
| | C12:0 | | | | G | T |
| | C14:0 | | | | G | T |
| 540 | C12:0 | 13 | rs386055644 | 63889282 | T | C |
| 541 | C6:0 | 13 | | 63986107 | G | A |
| | C8:0 | | | | G | A |
| | C10:0 | | | | G | A |
| | C12:0 | | | | G | A |
| | C14:0 | | | | G | A |
| 542 | C6:0 | 13 | | 64122383 | G | A |
| | C8:0 | | | | G | A |
| | C10:0 | | | | G | A |
| | C12:0 | | | | G | A |
| 543 | C8:0 | 13 | rs436218085 | 64233632 | T | TA |
| | C10:0 | | | | T | TA |
| | C12:0 | | | | T | TA |
| | C14:0 | | | | T | TA |
| | C18:1cis-9 | | | | TA | T |
| 544 | C6:0 | 13 | rs380237156 | 64326275 | G | A |
| | C8:0 | | | | G | A |
| | C10:0 | | | | G | A |
| | C12:0 | | | | G | A |
| | C14:0 | | | | G | A |
| 545 | C6:0 | 13 | | 64430137 | C | T |
| | C8:0 | | | | C | T |
| | C10:0 | | | | C | T |
| | C12:0 | | | | C | T |
| | C14:0 | | | | C | T |
| 546 | C6:0 | 13 | | 64437619 | T | G |
| | C8:0 | | | | T | G |
| | C10:0 | | | | T | G |
| | C12:0 | | | | T | G |
| | C14:0 | | | | T | G |
| 547 | C6:0 | 13 | rs379219552 | 64459763 | A | G |
| | C8:0 | | | | A | G |
| | C10:0 | | | | A | G |
| | C12:0 | | | | A | G |
| | C14:0 | | | | A | G |
| 548 | C8:0 | 13 | rs109108602 | 64493021 | G | T |
| | C14:0 | | | | G | T |
| 549 | C8:0 | 13 | rs109442789 | 64493180 | C | T |
| | C14:0 | | | | C | T |
| 550 | C8:0 | 13 | rs381918818 | 64493633 | G | T |
| | C14:0 | | | | G | T |
| 551 | C8:0 | 13 | rs457495420 | 64497075 | C | T |
| | C10:0 | | | | C | T |
| | C12:0 | | | | C | T |
| | C14:0 | | | | C | T |
| 552 | C14:0 | 13 | rs109417777 | 64498134 | G | T |
| 553 | C14:0 | 13 | rs207527222 | 64501066 | G | C |
| 554 | C6:0 | 13 | rs477403418 | 64507457 | C | T |
| | C8:0 | | | | C | T |
| | C10:0 | | | | C | T |
| | C12:0 | | | | C | T |
| | C14:0 | | | | C | T |
| 555 | C6:0 | 13 | rs461887762 | 64511038 | CAGA | C |
| | C8:0 | | | | CAGA | C |
| | C10:0 | | | | CAGA | C |
| | C12:0 | | | | CAGA | C |
| | C14:0 | | | | CAGA | C |
| 556 | C6:0 | 13 | rs449972870 | 64513840 | G | A |
| | C8:0 | | | | G | A |
| | C10:0 | | | | G | A |
| | C12:0 | | | | G | A |
| | C14:0 | | | | G | A |
| 557 | C6:0 | 13 | rs459399631 | 64516203 | TTA | T |
| | C8:0 | | | | TTA | T |
| | C10:0 | | | | TTA | T |
| | C12:0 | | | | TTA | T |
| | C14:0 | | | | TTA | T |
| 558 | C6:0 | 13 | rs459530428 | 64519771 | A | AC |
| | C8:0 | | | | A | AC |
| | C10:0 | | | | A | AC |
| | C12:0 | | | | A | AC |
| | C14:0 | | | | A | AC |
| 559 | C6:0 | 13 | rs449337366 | 64523547 | C | G |
| | C8:0 | | | | C | G |
| | C10:0 | | | | C | G |
| | C12:0 | | | | C | G |
| | C14:0 | | | | C | G |
| 560 | C6:0 | 13 | rs381037433 | 64523817 | G | GA |
| | C8:0 | | | | G | GA |
| | C10:0 | | | | G | GA |
| | C12:0 | | | | G | GA |
| | C14:0 | | | | G | GA |
| 561 | C6:0 | 13 | rs435926822 | 64523974 | G | A |
| | C8:0 | | | | G | A |
| | C10:0 | | | | G | A |
| | C12:0 | | | | G | A |
| | C14:0 | | | | G | A |
| 562 | C6:0 | 13 | rs459756383 | 64526071 | G | A |
| | C8:0 | | | | G | A |
| | C10:0 | | | | G | A |
| | C12:0 | | | | G | A |
| | C14:0 | | | | G | A |
| 563 | C6:0 | 13 | rs467272315 | 64527018 | C | G |
| | C8:0 | | | | C | G |
| | C10:0 | | | | C | G |
| | C12:0 | | | | C | G |
| | C14:0 | | | | C | G |
| 564 | C6:0 | 13 | rs446495267 | 64529334 | C | T |
| | C8:0 | | | | C | T |
| | C10:0 | | | | C | T |
| | C12:0 | | | | C | T |
| | C14:0 | | | | C | T |
| 565 | C6:0 | 13 | rs720522852 | 64529994 | C | T |
| | C8:0 | | | | C | T |
| | C10:0 | | | | C | T |
| | C12:0 | | | | C | T |
| | C14:0 | | | | C | T |
| 566 | C6:0 | 13 | rs480211939 | 64544921 | T | G |
| | C8:0 | | | | T | G |
| | C10:0 | | | | T | G |
| | C12:0 | | | | T | G |
| | C14:0 | | | | T | G |
| 567 | C6:0 | 13 | rs458143544 | 64550756 | T | C |
| | C8:0 | | | | T | C |
| | C10:0 | | | | T | C |
| | C12:0 | | | | T | C |
| | C14:0 | | | | T | C |
| 568 | C6:0 | 13 | | 64555339 | G | C |
| | C8:0 | | | | G | C |
| | C10:0 | | | | G | C |
| | C12:0 | | | | G | C |
| | C14:0 | | | | G | C |
| 569 | C6:0 | 13 | rs523108279 | 64568326 | C | T |
| | C8:0 | | | | C | T |
| | C10:0 | | | | C | T |
| | C12:0 | | | | C | T |
| | C14:0 | | | | C | T |
| 570 | C6:0 | 13 | | 64569790 | C | T |
| | C8:0 | | | | C | T |
| | C10:0 | | | | C | T |
| | C12:0 | | | | C | T |
| | C14:0 | | | | C | T |
| 571 | C6:0 | 13 | rs41700747 | 64632478 | T | A |
| | C8:0 | | | | T | A |
| | C10:0 | | | | T | A |
| | C12:0 | | | | T | A |
| | C14:0 | | | | T | A |
| 572 | C6:0 | 13 | rs473665961 | 64641380 | G | A |
| | C8:0 | | | | G | A |
| | C10:0 | | | | G | A |
| | C12:0 | | | | G | A |
| | C14:0 | | | | G | A |
| 573 | C6:0 | 13 | rs475365460 | 64641613 | G | A |
| | C8:0 | | | | G | A |
| | C10:0 | | | | G | A |
| | C12:0 | | | | G | A |
| | C14:0 | | | | G | A |
| 574 | C6:0 | 13 | rs384960422 | 64642051 | T | C |
| | C8:0 | | | | T | C |
| | C10:0 | | | | T | C |
| | C12:0 | | | | T | C |
| | C14:0 | | | | T | C |
| 575 | C6:0 | 13 | rs378971460 | 64642826 | G | C |
| | C8:0 | | | | G | C |
| | C10:0 | | | | G | C |
| | C12:0 | | | | G | C |
| | C14:0 | | | | G | C |
| 576 | C6:0 | 13 | rs384124018 | 64644542 | C | T |
| | C8:0 | | | | C | T |
| | C10:0 | | | | C | T |
| | C12:0 | | | | C | T |
| | C14:0 | | | | C | T |
| 577 | C6:0 | 13 | | 64654792 | TATAC | T |
| | C8:0 | | | | TATAC | T |
| | C10:0 | | | | TATAC | T |
| | C12:0 | | | | TATAC | T |
| | C14:0 | | | | TATAC | T |
| 578 | C6:0 | 13 | rs41700738 | 64655523 | C | T |
| | C8:0 | | | | C | T |
| | C10:0 | | | | C | T |
| | C12:0 | | | | C | T |
| | C14:0 | | | | C | T |
| 579 | C6:0 | 13 | | 64690602 | A | G |
| | C8:0 | | | | A | G |
| | C10:0 | | | | A | G |
| | C12:0 | | | | A | G |
| | C14:0 | | | | A | G |
| 580 | C6:0 | 13 | | 64729354 | C | T |
| | C8:0 | | | | C | T |
| | C10:0 | | | | C | T |
| | C12:0 | | | | C | T |
| | C14:0 | | | | C | T |
| 581 | C6:0 | 13 | | 64782240 | C | T |
| | C8:0 | | | | C | T |
| 582 | C6:0 | 13 | | 64837445 | G | A |
| | C8:0 | | | | G | A |
| 583 | C6:0 | 13 | | 64906333 | C | T |
| | C8:0 | | | | C | T |
| 584 | C6:0 | 13 | | 65032275 | C | T |
| | C8:0 | | | | C | T |
| | C14:0 | | | | C | T |
| 585 | C6:0 | 13 | | 65186495 | C | G |
| | C8:0 | | | | C | G |
| | C10:0 | | | | C | G |
| | C12:0 | | | | C | G |
| | C14:0 | | | | C | G |
| 586 | C8:0 | 13 | rs211165594 | 65200902 | A | G |
| | C10:0 | | | | A | G |
| | C12:0 | | | | A | G |
| | C14:0 | | | | A | G |
| 587 | C14:0 | 13 | rs43711986 | 65214101 | G | A |
| 588 | C12:0 | 13 | rs110083815 | 65243590 | G | A |
| | C14:0 | | | | G | A |
| | C18:1cis-9 | | | | A | G |
| 589 | C10:0 | 13 | rs109278546 | 65248121 | C | T |
| | C12:0 | | | | C | T |
| | C14:0 | | | | C | T |
| | C18:1cis-9 | | | | T | C |
| 590 | C6:0 | 13 | | 65296364 | A | G |
| | C8:0 | | | | A | G |
| | C10:0 | | | | A | G |
| | C12:0 | | | | A | G |
| | C14:0 | | | | A | G |
| 591 | C6:0 | 13 | rs522985070 | 65310168 | A | T |
| | C8:0 | | | | A | T |
| | C10:0 | | | | A | T |
| | C12:0 | | | | A | T |
| | C14:0 | | | | A | T |
| 592 | C6:0 | 13 | | 65321350 | G | T |
| | C8:0 | | | | G | T |
| | C10:0 | | | | G | T |
| | C12:0 | | | | G | T |
| | C14:0 | | | | G | T |
| 593 | C8:0 | 13 | | 65351040 | T | G |
| | C10:0 | | | | T | G |
| | C12:0 | | | | T | G |
| | C14:0 | | | | T | G |
| 594 | C8:0 | 13 | | 65487713 | C | G |
| | C12:0 | | | | C | G |
| | C14:0 | | | | C | G |
| 595 | C4:0 | 17 | rs210620686 | 52971285 | C | T |
| | C6:0 | | | | C | T |
| 596 | C4:0 | 17 | rs207997694 | 52971731 | T | C |
| | C6:0 | | | | T | C |
| 597 | C4:0 | 17 | rs211685274 | 52975212 | G | A |
| | C6:0 | | | | G | A |
| 598 | C4:0 | 17 | rs384356231 | 52981667 | T | C |
| 599 | C4:0 | 17 | rs385196649 | 52983677 | A | G |
| | C6:0 | | | | A | G |
| 600 | C4:0 | 17 | rs383939492 | 52988005 | T | C |
| | C6:0 | | | | T | C |
| 601 | C4:0 | 17 | rs209715690 | 52993411 | T | C |
| | C6:0 | | | | T | C |
| 602 | C4:0 | 17 | rs378897148 | 52995059 | T | C |
| | C6:0 | | | | T | C |
| 603 | C6:0 | 17 | | 52998445 | A | G |
| 604 | C4:0 | 17 | rs207984047 | 52999963 | T | A |
| | C6:0 | | | | T | A |
| 605 | C4:0 | 17 | rs41638794 | 53010785 | A | C |
| | C6:0 | | | | A | C |
| 606 | C4:0 | 17 | rs208784259 | 53021531 | C | T |
| | C6:0 | | | | C | T |
| 607 | C4:0 | 17 | rs379775146 | 53023240 | C | A |
| | C6:0 | | | | C | A |
| 608 | C4:0 | 17 | rs208914703 | 53025568 | G | A |
| | C6:0 | | | | G | A |
| 609 | C6:0 | 17 | rs465933786 | 53027601 | T | C |
| 610 | C6:0 | 17 | rs436736287 | 53028054 | A | G |
| 611 | C4:0 | 17 | rs382516208 | 53029882 | G | A |
| | C6:0 | | | | G | A |
| 612 | C6:0 | 17 | | 53032498 | A | C |
| 613 | C6:0 | 17 | | 53033208 | T | C |
| 614 | C6:0 | 17 | rs461037541 | 53034516 | G | A |
| 615 | C4:0 | 17 | rs446350814 | 53038789 | C | T |
| 616 | C4:0 | 17 | rs469684844 | 53041483 | C | A |
| 617 | C6:0 | 17 | rs470804340 | 53043920 | A | G |
| 618 | C6:0 | 17 | rs479802539 | 53049691 | G | A |
| 619 | C6:0 | 17 | rs449032230 | 53051338 | G | A |
| 620 | C6:0 | 17 | rs465071565 | 53052990 | A | G |
| 621 | C6:0 | 17 | rs481844245 | 53053021 | A | C |
| 622 | C4:0 | 17 | rs444541341 | 53053566 | T | A |
| 623 | C4:0 | 17 | rs381799511 | 53054196 | G | A |
| | C6:0 | | | | G | A |
| 624 | C4:0 | 17 | rs378454875 | 53054197 | G | A |
| | C6:0 | | | | G | A |
| 625 | C4:0 | 17 | | 53058249 | C | T |
| | C6:0 | | | | C | T |
| 626 | C6:0 | 17 | rs435036894 | 53065665 | T | C |
| 627 | C4:0 | 17 | rs521892164 | 53070436 | T | C |
| | C6:0 | | | | T | C |
| 628 | C6:0 | 17 | | 53071500 | TACACACA | T |
| | | | | | CACACACA | |
| | | | | | CACACACA | |
| | | | | | CACACAC | |
| | C8:0 | | | | TACACACA | T |
| | | | | | CACACACA | |
| | | | | | CACACACA | |
| | | | | | CACACAC | |
| | C10:0 | | | | TACACACA | T |
| | | | | | CACACACA | |
| | | | | | CACACACA | |
| | | | | | CACACAC | |
| 629 | C4:0 | 17 | rs384625792 | 53072209 | C | T |
| | C6:0 | | | | C | T |
| 630 | C6:0 | 17 | rs448501071 | 53072959 | T | C |
| 631 | C6:0 | 17 | rs444137034 | 53073328 | T | C |
| 632 | C6:0 | 17 | rs447613358 | 53073930 | C | T |
| 633 | C4:0 | 17 | rs432216053 | 53074086 | AAC | A |
| | C6:0 | | | | AAC | A |
| 634 | C4:0 | 17 | rs452468649 | 53074119 | A | G |
| | C6:0 | | | | A | G |
| 635 | C4:0 | 17 | rs437826696 | 53074186 | A | G |
| | C6:0 | | | | A | G |
| 636 | C4:0 | 17 | rs470578491 | 53074609 | A | G |
| | C6:0 | | | | A | G |
| 637 | C4:0 | 17 | rs209474685 | 53075027 | A | C |
| | C6:0 | | | | A | C |
| 638 | C4:0 | 17 | rs442299364 | 53075861 | T | C |
| | C6:0 | | | | T | C |
| 639 | C4:0 | 17 | rs209723941 | 53076386 | T | C |
| | C6:0 | | | | T | C |
| 640 | C4:0 | 17 | rs468384620 | 53076606 | G | C |
| | C6:0 | | | | G | C |
| 641 | C4:0 | 17 | rs455946322 | 53076818 | T | G |
| | C6:0 | | | | T | G |
| 642 | C4:0 | 17 | rs211126328 | 53076897 | T | C |
| | C6:0 | | | | T | C |
| 643 | C4:0 | 17 | rs208545225 | 53076931 | A | G |
| | C6:0 | | | | A | G |
| 644 | C4:0 | 17 | rs210323226 | 53076936 | T | C |
| | C6:0 | | | | T | C |
| 645 | C4:0 | 17 | rs471850752 | 53077298 | A | C |
| | C6:0 | | | | A | C |
| 646 | C4:0 | 17 | rs463715300 | 53077601 | T | C |
| | C6:0 | | | | T | C |
| 647 | C4:0 | 17 | rs479051665 | 53077974 | G | GT |
| | C6:0 | | | | G | GT |
| 648 | C4:0 | 17 | rs477658921 | 53078216 | G | GAAAGTGA |
| | C6:0 | | | | G | GAAAGTGA |
| 649 | C4:0 | 17 | rs211135086 | 53078316 | C | T |
| | C6:0 | | | | C | T |
| 650 | C4:0 | 17 | rs207911941 | 53078394 | T | C |
| | C6:0 | | | | T | C |
| 651 | C4:0 | 17 | rs210115102 | 53078460 | A | C |
| | C6:0 | | | | A | C |
| 652 | C4:0 | 17 | rs382245802 | 53078595 | C | T |
| | C6:0 | | | | C | T |
| 653 | C4:0 | 17 | rs451762433 | 53078815 | GT | G |
| 654 | C4:0 | 17 | rs209787441 | 53079293 | C | T |
| | C6:0 | | | | C | T |
| 655 | C4:0 | 17 | rs211616670 | 53079307 | A | G |
| | C6:0 | | | | A | G |
| 656 | C4:0 | 17 | rs208766232 | 53079405 | C | G |
| | C6:0 | | | | C | G |
| 657 | C4:0 | 17 | rs210631983 | 53079464 | A | C |
| | C6:0 | | | | A | C |
| 658 | C4:0 | 17 | rs211666757 | 53079468 | C | T |
| | C6:0 | | | | C | T |
| 659 | C4:0 | 17 | rs208735464 | 53079507 | C | T |
| | C6:0 | | | | C | T |
| 660 | C4:0 | 17 | rs448754537 | 53079557 | G | GT |
| | C6:0 | | | | G | GT |
| 661 | C4:0 | 17 | rs209241644 | 53079602 | C | G |
| | C6:0 | | | | C | G |
| 662 | C4:0 | 17 | rs452024516 | 53079609 | TC | T |
| | C6:0 | | | | TC | T |
| 663 | C4:0 | 17 | rs210089874 | 53079641 | T | A |
| | C6:0 | | | | T | A |
| 664 | C4:0 | 17 | rs210545931 | 53079660 | A | G |
| | C6:0 | | | | A | G |
| 665 | C4:0 | 17 | rs378278234 | 53079698 | A | T |
| | C6:0 | | | | A | T |
| 666 | C4:0 | 17 | rs474259058 | 53079752 | A | G |
| | C6:0 | | | | A | G |
| 667 | C4:0 | 17 | rs211135705 | 53079788 | G | A |
| | C6:0 | | | | G | A |
| 668 | C6:0 | 17 | rs441081928 | 53080179 | A | G |
| 669 | C4:0 | 17 | rs207630811 | 53080284 | A | G |
| | C6:0 | | | | A | G |
| 670 | C4:0 | 17 | rs41638800 | 53080800 | A | C |
| 671 | C4:0 | 17 | rs41638803 | 53080902 | G | A |
| 672 | C4:0 | 17 | rs211197242 | 53081441 | A | G |
| | C6:0 | | | | A | G |
| 673 | C4:0 | 17 | rs381484462 | 53081452 | T | C |
| | C6:0 | | | | T | C |
| 674 | C4:0 | 17 | rs381040511 | 53081745 | C | T |
| | C6:0 | | | | C | T |
| 675 | C4:0 | 17 | rs471482330 | 53081900 | T | A |
| 676 | C4:0 | 17 | rs379587739 | 53082256 | A | T |
| | C6:0 | | | | A | T |
| 677 | C4:0 | 17 | rs384944618 | 53089449 | C | T |
| | C6:0 | | | | C | T |
| 678 | C6:0 | 17 | rs477456528 | 53090245 | T | C |
| 679 | C4:0 | 17 | rs453170809 | 53091178 | T | G |
| 680 | C4:0 | 17 | rs445816688 | 53092921 | A | G |
| 681 | C4:0 | 17 | rs381627279 | 53092938 | C | T |
| | C6:0 | | | | C | T |
| 682 | C6:0 | 17 | rs136527636 | 53101097 | G | A |
| | C8:0 | | | | G | A |
| 683 | C8:0 | 17 | rs378585850 | 53102051 | AGAGCCT | A |
| 684 | C4:0 | 19 | rs110710863 | 37420803 | C | T |
| 685 | C4:0 | 19 | rs110201122 | 37420824 | T | C |
| 686 | C4:0 | 19 | rs110171922 | 37421093 | T | G |
| 687 | C4:0 | 19 | rs110370931 | 37421119 | A | G |
| 688 | C6:0 | 19 | rs110601471 | 37421292 | C | G |
| 689 | C4:0 | 19 | rs133598433 | 37421427 | T | C |
| | C6:0 | | | | T | C |
| 690 | C4:0 | 19 | rs109273604 | 37421447 | G | A |
| | C6:0 | | | | G | A |
| 691 | C4:0 | 19 | rs110609264 | 37421511 | A | G |
| | C6:0 | | | | A | G |
| 692 | C4:0 | 19 | rs476079746 | 37421626 | GAAAAAA | G |
| | C6:0 | | | | GAAAAAA | G |
| 693 | C4:0 | 19 | rs110416810 | 37421766 | A | C |
| | C6:0 | | | | A | C |
| 694 | C4:0 | 19 | rs137408608 | 37421789 | T | C |
| | C6:0 | | | | T | C |
| 695 | C4:0 | 19 | rs379413235 | 37421873 | T | C |
| | C6:0 | | | | T | C |
| 696 | C4:0 | 19 | rs382079281 | 37421874 | G | A |
| | C6:0 | | | | G | A |
| 697 | C4:0 | 19 | rs109168645 | 37421939 | C | A |
| | C6:0 | | | | C | A |
| 698 | C4:0 | 19 | rs137183595 | 37422196 | C | T |
| | C6:0 | | | | C | T |
| 699 | C4:0 | 19 | rs135812358 | 37422409 | C | G |
| | C6:0 | | | | C | G |
| 700 | C4:0 | 19 | rs133509820 | 37422432 | G | C |
| | C6:0 | | | | G | C |
| 701 | C4:0 | 19 | rs134576159 | 37422449 | C | T |
| | C6:0 | | | | C | T |
| 702 | C4:0 | 19 | rs136067775 | 37422461 | A | G |
| | C6:0 | | | | A | G |
| 703 | C4:0 | 19 | rs133648926 | 37422538 | A | G |
| | C6:0 | | | | A | G |
| 704 | C4:0 | 19 | rs134775921 | 37422561 | A | G |
| | C6:0 | | | | A | G |
| 705 | C4:0 | 19 | rs137767490 | 37422691 | C | A |
| | C6:0 | | | | C | A |
| 706 | C4:0 | 19 | rs109729348 | 37422810 | T | A |
| | C6:0 | | | | T | A |
| 707 | C4:0 | 19 | rs135001727 | 37422935 | T | C |
| | C6:0 | | | | T | C |
| 708 | C4:0 | 19 | rs133802021 | 37422972 | T | G |
| | C6:0 | | | | T | G |
| 709 | C4:0 | 19 | rs135728427 | 37423317 | A | G |
| | C6:0 | | | | A | G |
| 710 | C4:0 | 19 | rs133979612 | 37423324 | T | C |
| | C6:0 | | | | T | C |
| 711 | C4:0 | 19 | rs135123627 | 37423391 | CT | C |
| | C6:0 | | | | CT | C |
| 712 | C4:0 | 19 | rs134162730 | 37423657 | G | C |
| | C6:0 | | | | G | C |
| 713 | C4:0 | 19 | rs137382226 | 37423683 | C | T |
| | C6:0 | | | | C | T |
| 714 | C4:0 | 19 | rs109960587 | 37423701 | T | C |
| | C6:0 | | | | T | C |
| 715 | C4:0 | 19 | rs134677753 | 37423782 | G | A |
| | C6:0 | | | | G | A |
| 716 | C4:0 | 19 | rs133315299 | 37423848 | G | A |
| | C6:0 | | | | G | A |
| 717 | C4:0 | 19 | rs137181081 | 37424353 | C | G |
| | C6:0 | | | | C | G |
| 718 | C4:0 | 19 | rs135802186 | 37424369 | A | C |
| | C6:0 | | | | A | C |
| 719 | C4:0 | 19 | rs136803646 | 37424464 | C | T |
| | C6:0 | | | | C | T |
| 720 | C4:0 | 19 | rs135719507 | 37425463 | C | G |
| | C6:0 | | | | C | G |
| 721 | C4:0 | 19 | rs134757858 | 37425768 | T | G |
| | C6:0 | | | | T | G |
| 722 | C4:0 | 19 | rs135912187 | 37425818 | G | A |
| | C6:0 | | | | G | A |
| 723 | C4:0 | 19 | rs133675471 | 37426461 | G | A |
| | C6:0 | | | | G | A |
| 724 | C4:0 | 19 | rs137377991 | 37426466 | T | G |
| | C6:0 | | | | T | G |
| 725 | C4:0 | 19 | rs133655171 | 37426504 | C | CT |
| | C6:0 | | | | C | CT |
| 726 | C4:0 | 19 | rs136258047 | 37426562 | T | C |
| | C6:0 | | | | T | C |
| 727 | C4:0 | 19 | rs136959782 | 37426570 | A | G |
| | C6:0 | | | | A | G |
| 728 | C4:0 | 19 | rs135043167 | 37426743 | T | C |
| | C6:0 | | | | T | C |
| 729 | C4:0 | 19 | rs134134839 | 37427073 | G | A |
| | C6:0 | | | | G | A |
| 730 | C4:0 | 19 | rs134357578 | 37427295 | T | C |
| | C6:0 | | | | T | C |
| 731 | C4:0 | 19 | rs135860451 | 37427334 | T | C |
| | C6:0 | | | | T | C |
| 732 | C4:0 | 19 | rs132850606 | 37427373 | A | C |
| | C6:0 | | | | A | C |
| 733 | C4:0 | 19 | rs134153528 | 37427512 | C | A |
| | C6:0 | | | | C | A |
| 734 | C4:0 | 19 | rs137607495 | 37427695 | A | G |
| | C6:0 | | | | A | G |
| 735 | C4:0 | 19 | rs137124042 | 37427696 | C | T |
| | C6:0 | | | | C | T |
| 736 | C4:0 | 19 | rs135479468 | 37427811 | T | C |
| | C6:0 | | | | T | C |
| 737 | C4:0 | 19 | rs208812779 | 37427934 | C | T |
| | C6:0 | | | | C | T |
| 738 | C4:0 | 19 | rs210462806 | 37427947 | T | C |
| | C6:0 | | | | T | C |
| 739 | C4:0 | 19 | rs211367926 | 37427957 | A | G |
| | C6:0 | | | | A | G |
| 740 | C4:0 | 19 | rs380334338 | 37427983 | G | A |
| | C6:0 | | | | G | A |
| 741 | C4:0 | 19 | rs378112208 | 37428145 | T | A |
| | C6:0 | | | | T | A |
| 742 | C4:0 | 19 | rs381773378 | 37428203 | G | A |
| | C6:0 | | | | G | A |
| 743 | C4:0 | 19 | rs384061903 | 37428214 | G | A |
| | C6:0 | | | | G | A |
| 744 | C4:0 | 19 | rs381452316 | 37428217 | G | A |
| | C6:0 | | | | G | A |
| 745 | C4:0 | 19 | rs454426041 | 37428406 | T | TGCTG |
| 746 | C4:0 | 19 | rs133864523 | 37428532 | A | G |
| | C6:0 | | | | A | G |
| 747 | C4:0 | 19 | rs109760848 | 37428964 | G | C |
| | C6:0 | | | | G | C |
| 748 | C4:0 | 19 | rs136957290 | 37429174 | T | C |
| | C6:0 | | | | T | C |
| 749 | C4:0 | 19 | rs135031930 | 37429200 | G | A |
| | C6:0 | | | | G | A |
| 750 | C4:0 | 19 | rs136577796 | 37429299 | C | T |
| | C6:0 | | | | C | T |
| 751 | C4:0 | 19 | rs134461158 | 37429505 | C | T |
| | C6:0 | | | | C | T |
| 752 | C4:0 | 19 | rs470727529 | 37429535 | GTCA | G |
| | C6:0 | | | | GTCA | G |
| 753 | C4:0 | 19 | rs132894782 | 37429584 | T | C |
| | C6:0 | | | | T | C |
| 754 | C4:0 | 19 | rs379929109 | 37429611 | G | A |
| | C6:0 | | | | G | A |
| 755 | C4:0 | 19 | rs207642909 | 37429715 | G | C |
| | C6:0 | | | | G | C |
| 756 | C4:0 | 19 | rs377908592 | 37429731 | C | T |
| | C6:0 | | | | C | T |
| 757 | C4:0 | 19 | rs385022671 | 37429766 | G | A |
| | C6:0 | | | | G | A |
| 758 | C4:0 | 19 | rs384668978 | 37429842 | C | T |
| | C6:0 | | | | C | T |
| 759 | C4:0 | 19 | rs381945799 | 37429893 | C | G |
| | C6:0 | | | | C | G |
| 760 | C4:0 | 19 | rs378103579 | 37430038 | G | A |
| | C6:0 | | | | G | A |
| 761 | C4:0 | 19 | rs379612849 | 37430398 | T | C |
| | C6:0 | | | | T | C |
| 762 | C4:0 | 19 | rs209313162 | 37430599 | G | A |
| | C6:0 | | | | G | A |
| 763 | C4:0 | 19 | rs137694497 | 37430691 | T | C |
| | C6:0 | | | | T | C |
| 764 | C4:0 | 19 | rs133969987 | 37430745 | G | A |
| | C6:0 | | | | G | A |
| 765 | C4:0 | 19 | rs208203050 | 37431116 | G | C |
| | C6:0 | | | | G | C |
| 766 | C4:0 | 19 | rs209459815 | 37431323 | C | T |
| | C6:0 | | | | C | T |
| 767 | C6:0 | 19 | rs134974631 | 37431439 | A | AGTAT |
| 768 | C4:0 | 19 | rs134982853 | 37431723 | C | G |
| | C6:0 | | | | C | G |
| 769 | C4:0 | 19 | rs132959868 | 37431903 | G | A |
| | C6:0 | | | | G | A |
| 770 | C4:0 | 19 | rs135144890 | 37432217 | A | G |
| 771 | C4:0 | 19 | rs136585582 | 37432258 | G | A |
| | C6:0 | | | | G | A |
| 772 | C4:0 | 19 | rs134430106 | 37432259 | G | A |
| 773 | C6:0 | 19 | rs136278512 | 37432404 | G | A |
| | C4:0 | | | | C | T |
| | C6:0 | | | | C | T |
| 774 | C4:0 | 19 | rs133391765 | 37432454 | C | T |
| | C6:0 | | | | C | T |
| 775 | C4:0 | 19 | rs134565972 | 37432565 | A | C |
| | C6:0 | | | | A | C |
| 776 | C4:0 | 19 | rs136065115 | 37432605 | A | G |
| | C6:0 | | | | A | G |
| 777 | C4:0 | 19 | rs133641031 | 37432642 | A | G |
| | C6:0 | | | | A | G |
| 778 | C4:0 | 19 | rs135308492 | 37433580 | T | C |
| | C6:0 | | | | T | C |
| 779 | C4:0 | 19 | rs136163473 | 37433644 | A | G |
| | C6:0 | | | | A | G |
| 780 | C4:0 | 19 | rs134866950 | 37433917 | A | G |
| | C6:0 | | | | A | G |
| 781 | C4:0 | 19 | rs137830904 | 37434273 | A | T |
| | C6:0 | | | | A | T |
| 782 | C4:0 | 19 | rs133758652 | 37434308 | G | A |
| | C6:0 | | | | G | A |
| 783 | C4:0 | 19 | rs135079368 | 37434328 | A | C |
| | C6:0 | | | | A | C |
| 784 | C4:0 | 19 | rs135793795 | 37434398 | C | T |
| | C6:0 | | | | C | T |
| 785 | C4:0 | 19 | rs134005788 | 37434407 | C | A |
| | C6:0 | | | | C | A |
| 786 | C4:0 | 19 | rs133547101 | 37434481 | A | G |
| | C6:0 | | | | A | G |
| 787 | C4:0 | 19 | rs132832565 | 37434487 | A | G |
| | C6:0 | | | | A | G |
| 788 | C4:0 | 19 | rs137090886 | 37434518 | T | C |
| | C6:0 | | | | T | C |
| 789 | C4:0 | 19 | rs136064330 | 37435003 | T | TCTGA |
| | C6:0 | | | | T | TCTGA |
| 790 | C4:0 | 19 | rs136671929 | 37435359 | G | A |
| | C6:0 | | | | G | A |
| 791 | C4:0 | 19 | rs134672095 | 37435570 | T | C |
| | C6:0 | | | | T | C |
| 792 | C4:0 | 19 | rs136283595 | 37435651 | C | T |
| | C6:0 | | | | C | T |
| 793 | C4:0 | 19 | rs137840008 | 37435743 | A | AAT |
| | C6:0 | | | | A | AAT |
| 794 | C4:0 | 19 | rs133591435 | 37435788 | C | CCACAAAA |
| | | | | | | TGAGTTTT |
| | | | | | | GG |
| | C6:0 | | | | C | CCACAAAA |
| | | | | | | TGAGTTTT |
| | | | | | | GG |
| 795 | C4:0 | 19 | rs135485508 | 37435982 | T | C |
| | C6:0 | | | | T | C |
| 796 | C4:0 | 19 | rs134871207 | 37436054 | C | T |
| | C6:0 | | | | C | T |
| 797 | C4:0 | 19 | rs137633584 | 37436062 | A | G |
| | C6:0 | | | | A | G |
| 798 | C4:0 | 19 | rs133408506 | 37436088 | A | G |
| | C6:0 | | | | A | G |
| 799 | C4:0 | 19 | rs137244033 | 37436090 | T | A |
| | C6:0 | | | | T | A |
| 800 | C4:0 | 19 | rs135981635 | 37436245 | G | A |
| | C6:0 | | | | G | A |
| 801 | C4:0 | 19 | rs133519775 | 37436425 | C | G |
| | C6:0 | | | | C | G |
| 802 | C4:0 | 19 | rs134784649 | 37436570 | C | T |
| | C6:0 | | | | C | T |
| 803 | C4:0 | 19 | rs135868292 | 37436588 | A | G |
| | C6:0 | | | | A | G |
| 804 | C4:0 | 19 | rs135105101 | 37436847 | T | TTGGTTCC |
| | | | | | | CAAGACAG |
| | C6:0 | | | | T | TTGGTTCC |
| | | | | | | CAAGACAG |
| 805 | C4:0 | 19 | rs133602694 | 37436886 | C | T |
| | C6:0 | | | | C | T |
| 806 | C4:0 | 19 | rs134512402 | 37436977 | C | T |
| | C6:0 | | | | C | T |
| 807 | C4:0 | 19 | rs137828242 | 37436988 | C | G |
| | C6:0 | | | | C | G |
| 808 | C4:0 | 19 | rs133799248 | 37437026 | A | G |
| | C6:0 | | | | A | G |
| 809 | C4:0 | 19 | rs133883757 | 37437039 | A | ACC |
| | C6:0 | | | | A | ACC |
| 810 | C4:0 | 19 | rs132715652 | 37437163 | T | C |
| | C6:0 | | | | T | C |
| 811 | C4:0 | 19 | rs110189669 | 37437563 | G | A |
| | C6:0 | | | | G | A |
| 812 | C4:0 | 19 | rs109530290 | 37437573 | G | T |
| | C6:0 | | | | G | T |
| 813 | C4:0 | 19 | rs41576369 | 37437695 | G | A |
| | C6:0 | | | | G | A |
| 814 | C4:0 | 19 | rs41576370 | 37437821 | A | G |
| 815 | C4:0 | 19 | rs135468541 | 37438010 | C | T |
| | C6:0 | | | | C | T |
| 816 | C4:0 | 19 | rs136678786 | 37438105 | T | G |
| 817 | C4:0 | 19 | rs134336883 | 37438112 | T | C |
| 818 | C4:0 | 19 | rs137607056 | 37438113 | G | A |
| | C6:0 | | | | G | A |
| 819 | C4:0 | 19 | rs133108726 | 37438114 | T | G |
| | C6:0 | | | | T | G |
| 820 | C4:0 | 19 | rs134873693 | 37438226 | G | A |
| | C6:0 | | | | G | A |
| 821 | C4:0 | 19 | rs135640211 | 37438269 | A | G |
| | C6:0 | | | | A | G |
| 822 | C4:0 | 19 | rs133351905 | 37438337 | A | C |
| | C6:0 | | | | A | C |
| 823 | C4:0 | 19 | rs137241977 | 37438365 | T | C |
| | C6:0 | | | | T | C |
| 824 | C4:0 | 19 | rs135974828 | 37438383 | C | G |
| | C6:0 | | | | C | G |
| 825 | C4:0 | 19 | rs137028398 | 37438429 | A | G |
| 826 | C4:0 | 19 | rs110201546 | 37438468 | G | C |
| | C6:0 | | | | G | C |
| 827 | C4:0 | 19 | rs136172294 | 37438784 | C | T |
| | C6:0 | | | | C | T |
| 828 | C4:0 | 19 | rs133860679 | 37438863 | A | C |
| | C6:0 | | | | A | C |
| 829 | C4:0 | 19 | rs135538891 | 37438870 | A | G |
| | C6:0 | | | | A | G |
| 830 | C4:0 | 19 | rs133066628 | 37438939 | A | G |
| | C6:0 | | | | A | G |
| 831 | C4:0 | 19 | rs137103770 | 37438958 | AG | A |
| | C6:0 | | | | AG | A |
| 832 | C4:0 | 19 | rs133819098 | 37439297 | A | G |
| 833 | C4:0 | 19 | rs137438705 | 37439300 | C | G |
| 834 | C4:0 | 19 | rs132754289 | 37439381 | C | T |
| 835 | C4:0 | 19 | rs137181696 | 37439454 | G | C |
| 836 | C4:0 | 19 | rs135564283 | 37439496 | A | G |
| 837 | C4:0 | 19 | rs134154369 | 37439746 | T | C |
| 838 | C4:0 | 19 | rs135333508 | 37439816 | C | T |
| 839 | C4:0 | 19 | rs136839550 | 37439830 | T | C |
| 840 | C4:0 | 19 | rs134774547 | 37439869 | T | A |
| 841 | C4:0 | 19 | rs41576372 | 37439918 | C | G |
| 842 | C4:0 | 19 | rs135774487 | 37440020 | C | CA |
| 843 | C4:0 | 19 | rs41576371 | 37440101 | A | T |
| 844 | C4:0 | 19 | rs137347913 | 37440250 | C | G |
| 845 | C4:0 | 19 | rs136518355 | 37440578 | G | A |
| 846 | C4:0 | 19 | rs133518147 | 37440629 | C | A |
| 847 | C4:0 | 19 | rs135080269 | 37440705 | G | C |
| 848 | C4:0 | 19 | rs137036091 | 37440870 | T | C |
| 849 | C4:0 | 19 | rs137757787 | 37441036 | A | G |
| 850 | C4:0 | 19 | rs133731495 | 37441042 | A | G |
| 851 | C4:0 | 19 | rs134981038 | 37441076 | T | A |
| 852 | C4:0 | 19 | rs132957417 | 37441125 | T | A |
| 853 | C4:0 | 19 | rs134239990 | 37441243 | T | A |
| 854 | C4:0 | 19 | rs135142449 | 37441273 | A | G |
| 855 | C4:0 | 19 | rs132748248 | 37441281 | T | C |
| 856 | C4:0 | 19 | rs137179189 | 37441306 | C | T |
| 857 | C4:0 | 19 | rs135794148 | 37441360 | C | G |
| 858 | C4:0 | 19 | rs136685374 | 37441365 | C | CA |
| 859 | C4:0 | 19 | rs136090847 | 37441412 | C | G |
| 860 | C4:0 | 19 | rs135345504 | 37441479 | G | A |
| 861 | C4:0 | 19 | rs136451321 | 37441591 | C | T |
| 862 | C4:0 | 19 | rs133208066 | 37441708 | C | T |
| 863 | C4:0 | 19 | rs137212631 | 37441806 | A | G |
| 864 | C4:0 | 19 | rs136257382 | 37441808 | G | A |
| 865 | C4:0 | 19 | rs136959108 | 37442365 | A | C |
| 866 | C4:0 | 19 | rs134396590 | 37442684 | C | CA |
| 867 | C4:0 | 19 | rs135065747 | 37442917 | T | C |
| 868 | C4:0 | 19 | rs135788452 | 37442938 | G | A |
| 869 | C4:0 | 19 | rs134002361 | 37442979 | C | T |
| 870 | C4:0 | 19 | rs136223156 | 37443245 | C | CCTG |
| 871 | C4:0 | 19 | rs135205937 | 37443292 | C | T |
| 872 | C4:0 | 19 | rs472388394 | 37443331 | T | TGGGATTC |
| | | | | | | TCTA |
| | C6:0 | | | | T | TGGGATTC |
| | | | | | | TCTA |
| 873 | C4:0 | 19 | rs136613713 | 37443426 | G | A |
| 874 | C4:0 | 19 | rs134151301 | 37443497 | A | T |
| 875 | C4:0 | 19 | rs133021715 | 37443823 | C | T |
| 876 | C4:0 | 19 | rs134773341 | 37443851 | A | G |
| 877 | C4:0 | 19 | rs136303013 | 37444043 | A | G |
| 878 | C4:0 | 19 | rs133271003 | 37444088 | T | C |
| 879 | C4:0 | 19 | rs134904840 | 37444601 | A | G |
| 880 | C4:0 | 19 | rs137789177 | 37444654 | G | A |
| 881 | C4:0 | 19 | rs136810376 | 37444705 | T | G |
| 882 | C4:0 | 19 | rs134580708 | 37444849 | A | G |
| 883 | C4:0 | 19 | rs137534572 | 37445264 | C | T |
| 884 | C4:0 | 19 | rs133511250 | 37445303 | G | A |
| 885 | C4:0 | 19 | rs134781037 | 37445766 | C | G |
| 886 | C4:0 | 19 | rs135866327 | 37445876 | G | A |
| 887 | C12:0 | 19 | rs41921229 | 51307470 | A | T |
| 888 | C12:0 | 19 | rs41921230 | 51307589 | T | G |
| 889 | C10:0 | 19 | rs135377429 | 51308704 | T | A |
| | C12:0 | | | | T | A |
| | C14:0 | | | | T | A |
| 890 | C10:0 | 19 | rs41922135 | 51310989 | T | C |
| | C12:0 | | | | T | C |
| | C14:0 | | | | T | C |
| 891 | C10:0 | 19 | rs41922136 | 51311036 | T | G |
| | C12:0 | | | | T | G |
| | C14:0 | | | | T | G |
| 892 | C14:0 | 19 | rs41636874 | 51312886 | G | A |
| 893 | C12:0 | 19 | rs41921153 | 51317626 | G | C |
| 894 | C12:0 | 19 | rs41921154 | 51317647 | T | C |
| 895 | C12:0 | 19 | rs41921156 | 51318839 | T | C |
| 896 | C12:0 | 19 | rs41921157 | 51319275 | C | T |
| 897 | C12:0 | 19 | rs41921178 | 51326867 | T | C |
| 898 | C12:0 | 19 | rs41921180 | 51327163 | A | G |
| 899 | C10:0 | 19 | rs136995961 | 51327392 | C | T |
| | C12:0 | | | | C | T |
| | C14:0 | | | | C | T |
| 900 | C10:0 | 19 | rs382822995 | 51328364 | TC | T |
| | C12:0 | | | | TC | T |
| | C14:0 | | | | TC | T |
| 901 | C12:0 | 19 | rs41921183 | 51329756 | T | C |
| 902 | C10:0 | 19 | rs134213494 | 51329757 | G | A |
| | C12:0 | | | | G | A |
| | C14:0 | | | | G | A |
| 903 | C10:0 | 19 | rs457952543 | 51334328 | C | CT |
| | C12:0 | | | | C | CT |
| | C14:0 | | | | C | CT |
| | C18:1cis-11 | | | | CT | C |
| 904 | C10:0 | 19 | rs135163996 | 51342769 | G | GT |
| | C12:0 | | | | G | GT |
| | C14:0 | | | | G | GT |
| 905 | C10:0 | 19 | rs210011361 | 51343102 | T | A |
| | C12:0 | | | | T | A |
| | C14:0 | | | | T | A |
| 906 | C10:0 | 19 | rs136902713 | 51347031 | A | G |
| | C12:0 | | | | A | G |
| | C14:0 | | | | A | G |
| 907 | C10:0 | 19 | rs135090927 | 51349516 | C | G |
| | C12:0 | | | | C | G |
| | C14:0 | | | | C | G |
| 908 | C10:0 | 19 | rs210269014 | 51367626 | A | G |
| | C12:0 | | | | A | G |
| | C14:0 | | | | A | G |
| 909 | C10:0 | 19 | rs137787102 | 51372470 | C | T |
| | C12:0 | | | | C | T |
| | C14:0 | | | | C | T |
| 910 | C10:0 | 19 | rs135860613 | 51373688 | T | C |
| | C12:0 | | | | T | C |
| | C14:0 | | | | T | C |
| 911 | C10:0 | 19 | rs109016955 | 51381233 | G | C |
| | C12:0 | | | | G | C |
| | C14:0 | | | | G | C |
| | C18:1cis-10 | | | | C | G |
| 912 | C10:0 | 19 | rs136067046 | 51383847 | C | G |
| | C12:0 | | | | C | G |
| | C14:0 | | | | C | G |
| 913 | C10:0 | 19 | rs110674576 | 51386344 | A | G |
| | C12:0 | | | | A | G |
| | C14:0 | | | | A | G |
| 914 | C10:0 | 19 | rs137117849 | 51388187 | G | A |
| | C12:0 | | | | G | A |
| | C14:0 | | | | G | A |
| 915 | C10:0 | 19 | rs110687534 | 51388582 | G | A |
| | C12:0 | | | | G | A |
| | C14:0 | | | | G | A |
| 916 | C10:0 | 19 | rs446880702 | 51410942 | T | C |
| | C12:0 | | | | T | C |
| | C14:0 | | | | T | C |
| | C18:1cis-9 | | | | C | T |

TABLE 2
Polymorphisms including flanking sequences. A = Adenine, G = Guanine; C = Cytosine, T = Thymine. ″n″ indicates the polymorphic site,
″n1″ represents one of the alternative forms of the polymorphism and ″n2″ represents the other alternative form of the polymorphism.

| P# | | | |
|---|---|---|---|
| SEQ | | | |
| ID | | | |
| NO | Flanking Sequence | n1 | n2 |
| 1 | TCTATATCCCCTTAGTCCCACTTAAGTAAAGTTCAACACTAGCCGGTAGCATACTCAGTnAGTGGTGGGTAGGATGATAATGCAAGAGAGCTGACCAAAGAAATACAACAACAACGGCAA | A | C |
| 2 | TTTAAGTTTCTGATTTACAGATGTCTTCCCAGAAAATGATAATGGTGTAAAGAAAGATAnACGTCGACATTCTATTCTTGGTTCTTATCAAATAGAGGAAGGTCTGGCTAAGGGTCAGGG | A | C |
| 3 | CACCTCAGAAGTGACACAGTCCAGACAGGCTACCTGTCTCCTTATTTAAAAAAGGGGGGnAAAAGGGCTTTCTTTGTTTCAGTTTTAGAGGCAGTAAATACCTTTGCCTTGGAGCTATCC | A | G |
| 4 | ACTCAAAGCACCTTGATTTGTAATAGCCAAAAACTGGAAACAACCCAAATTGTCCATCCnCAAGTGAATGCATGTACAAACTGTAGTATACTTGAACAACAGAACACTATACAGAAGTAC | A | T |
| 5 | AATATAAATCAGGTATTAAAAGTGATGTACGAGAATAAAATACATGAAGGGATAGTAAGnTGTTTGTTATTTTAGATAAATAATTTAAGAAAGACAGCTATCCACCCATGTCCACATTAT | A | G |
| 6 | CCTTTTGGGGGCTTGGTGTGTGGTTTGGCCCTGAAGGCCTACTGAGTTATGTAGGGCTGnCTGTGTCTACACCTGTGATTCTCAAACTGCCCTTTAGGAAAGAATCACCCTGTATGCTTG | T | C |
| 7 | CATACAATGAAAATTTATAGGCTTTAGATTTGGATAGAACTGAATTAAAGTTTTGCTCAnAATCGCTTGCTTAACTGTGTGGCTCTGGGTAATAAATAAGGTGGAAAGTACATTTTTTGA | C | A |
| 8 | GCAATACTTTCTCGTTGCTATGTTGTATTCCTCCTATTCATAATACCAATGATGGATATnAGGATTATTTCTAGTTTTTAGCTTTCAGGACAAAGATGGCTGAGAAAATTCCTGCATACA | A | G |
| 9 | TTATCTTGGTATATAAGCTACCCCTTCCTGGAATGTCTTCCTTCCAAATTCACTTCTTTnTATGTCCTCCCCTTCTGAATTCACATCTTTAAAATGCCCTCCATTTCTTAGATCAGGCTT | T | A |
| 10 | TTTTGGCCAATCCAATAAAATAATCCCAAGAAAGAATAGATAGATAAGGATATGAAAGAnTGAGCAAAGTAAGAAGTGTACCCACAGAGTTTAGTTAATTTTACTATTTTCTTTGATGAT | G | A |
| 11 | ACCTTGAGAGCAGTACACTAAGTGAGATAAGCTACACAGAGAAAGATGAAAACTCTGTGnTATCACTAATATGTGGACTCTAAAAATCCTGAACTCACAGAAAGAGACAAAATGATGGTT | G | A |
| 12 | TTGTTAAACCATTAGTAACGCTGCCATGGTGGGAGTATTGACACTGTGGAAATTGGCAAnTACTACAAATTAGAGTTTGTTTTTTTGTTTTGTTTTGTTTTGTTTTCCCCAGAGAGCTGC | G | A |
| 13 | GCATTTGGCAGGCAGGTTCTTTACCACTGTGTCACCTGGAAGCCTAACGTTTCAGTACCnTAACAGAAATCGGAAAGTGTAAAAATCTTTAAAGACACCATCTTTCATGATTGAAAGAAA | A | G |
| 14 | TAATAATATGAGATAAAGTCCGAGTCTGAGGGGGTATTAAATGTACTCTCAAACTGGTTnTCTAGCAATTACAGCGTTGTCAGGATAATAAACAGGGGTGCTATTCTGGGTAATAAAGCA | C | T |
| 15 | CCTAAATCCTGACCCTCTGCTGTCCAGAGCCCCGGGCAGCTCCTGGTGGCAGTAACACTnGTCCTCTCAGCCTGGGTCACCCTTGATGGAGGGTGGACGAAGCAGAGTCCTGAGCTGCGG | A | G |
| 16 | TCAACACAGATGGGGAGATGTAAGCCTGGAGCTGGAGGAGCACCTGGCTAGGGTCTAAAnGCAAGTCAGAGGTGGGCGGTGGAGCCTGGGGTCTCAGCCTCAGACCCCCCAGGCCCCTGT | G | A |
| 17 | CATCATAACTGTAATGCTCGATCATGGTTGCTCGCTCTGCTAGAGACCGTCTCTCTTCCnTGTGATCCTGTGGGAGCATTAGTGAGTGACTAAGGACTGAGATTTCAGGGTCCTATCCAT | C | T |
| 18 | TTATGAGGAAACTGAGGCTCTGAGAGGCAAAAAGAAGTTGCCCTGGTTCAAAAGAAAGTnAAAGCTTCTCTCCCAGTTTTGTCCTTATTTCCCACAGATGTCTTCCATGCTCATAGTCAT | C | A |
| 19 | CTGACTACTACATTAACATCTCCTTCCCCATGATATTGATTTTAGGTATAATGAATCTCnCGGAATGATGAGAAATTCACAAAGATTTTATCAAGTCTACACTATTCAAAATATGACAAG | T | C |
| 20 | TATGAGTGGGACAGACACGCTAGACAGAGTTCTCCATACACAGGGAACTCAGTGAACTCnGGGTTATCACTCTACTGTTTTGAAGATAGATTTTATACTAAGGAGTACTGTGTGGGGCTC | G | A |
| 21 | TTACCACTAGCGCCACCTAGGAAGCACCAGCTGTAAAGTCACCATGGATTTTAAACTCAnAGAGGGTAGGCACCCCTAAACTCCTCAACTTTCAAGAGGCAACTGTATTTGCATACAATC | A | G |
| 22 | ATCCTTGGCAAAGCACTTGCTTCTATTTCAGTGAGCATCTATCATTTTTCTATTAAGAAnGGAAAGTAAGCTCTCAGATAATGTCGTCTACCTTTTCAGTAATAGTCTCATTGTTGTTTA | A | G |
| 23 | GGAATGCTAAAGGCAAATCTTCATAGTGTCCAGCTATCCCTATAGGGAAATCCATTCAAnTTAACTTTAAAAACTTACTCTCCTGGGGACAGAAATAAGAACTAGACACTATGTGATCAA | A | C |
| 24 | GCTATGCTTCACCTTTGCATTTTCACATGATTTTCAGAATAAGCTTATGGACAAAAATGnATCTACTATGACTCTGAGATGGCATTGAATTTATCAATCAATTTGGGAAATGTTTAAGAA | C | T |
| 25 | AGCACCTTCCACTCCACAGCACACAGATGCAACACCTGGACATCAGCAGCACAACTCCCnATCAAGAAACTTGCTGTTAAAAAAAAAAAATCCATGAAGAGTAATGGGATGGAATGGCTG | C | A |
| 26 | AAAGATGCTCAACATTGCTGATTATCAGAGAAATGCAAATCAAAACTACAGTGAGGCACnATCTCACACCCGTCAGAATGGCTACCACTGAAAAGTCTACAAATAACAAATGCTGGAGAG | G | C |
| 27 | TCCCCACCTAGTTCATGCTCTCCTGAAATGCTCATGGTCAGAGCAGCAGAAACAGGGATnGGTGGCTGAACTCCAAATACCTAAAGGAGCCCCCAGCCAGTGGAGAGTTATCTGTATGCA | A | G |
| 28 | TATGGGGAATGGGTCCATGCTGCGTCATTGAACAGATTATTTATCTTTTTGAAAAGGTCnTGTATGTTTAGCGTGAAGATTCATCAGGAGCAGAAAATATAAAATGAAAAGTAAGCTTCC | A | G |
| 29 | TCCCGAAGAGACGCCGCTGTGCTTGTTAGCTTTAAGAAGCACACAGCTTTATTGACAAAnGCATCACCTCCATAACTAAGAAAAGGGGTGATCGACAATTTCATATTTCCACTTTCAGGG | A | G |
| 30 | AAAACACAGTCTTGCATTTTAAAACTCGGCAATAAATACGAGAAATCCATTCATTGAGAnCCATCTTCATTACTGAAATGGAAATAGATTCACTGAGACATTATTGACTATCTCACGAGA | G | A |
| 31 | AGGGGTAGGTGAGGGAGAAGAAGGTGCCAGGCTGATCACCTAGGGCTGGTGACGAGTTTnGGGTTTTATTATCTGTGCTAGAGGCTAGTAATGAGGTTTATGTGAAGTGATGTGATGGGG | G | T |
| 32 | TTTATTGACACAGGGCCACACACACCTGTTGAGGAATTACCATGGCAGCTTTTCTGTTAnAACAGCAGAGTTGAATAGTCATGACAAACCGCAAAGCTTGACATATTCACCATCTGGTCC | C | A |
| 33 | AGCAGCTAAGCACAGCACAGGGTTGCCTGTAGTTTTCCAGTAGAGCATGGGTTTCTGAAnCCATCGTTAGAAGAGGCCCCAAGTTGCTATCTGAGCACTGGGTTCAGCCATCACTTCTGC | G | A |
| 34 | GGCATTTTGTCTGTCTGTAGTTGTTAACTATTACAGGCAATGCTGTAAGTGTTATCTGTnTAGTCTTAGAAGTGGCAAAAGCCTCCTTAGGTCATCAGTATTGTCCTCCTTGCCCTCTTG | A | G |
| 35 | ACCTGTTAGCTTCCTGCCCTTTTTAAATGAGTTATCTAATCTCCTTATACCGTACCTTAnTTATTTGTAAGTAAATAATGGTGTTGGCTTCCCAGCCCGGTGGTAAGGAATCAGCCCGCC | A | G |
| 36 | TTTAAAAAAATAGCTTTTGAAAAGATTTCAGGTAATTTAATTGGTATTTCTATTTACTTnCTCAGTTTGGTCTTAAATACTTTATATTGGTGTTTAAAGTTCTCCATTATTAGGAAGAAA | A | C |
| 37 | TGTGTTTTCTGGAATATTAGGGAATATGGTAGTGAAGAGTTCTAACCTTGAGGCTATTTnTCCAAGTTAAAATGGTCTTGAAGATTCCCATTAAGCTCGAAGGTTGGGAACTGGGTGGGT | C | A |
| 38 | TTAGGAAAGGGGCAAAAATGAGCCACTTTACAGTGGAGAAATTCAAATAATACTACCTTnTCCATGTTCTCAATATCACCATCAACAGTGATGTTGGTAGTGTGTATCCTTGATATAATG | T | A |
| 39 | GAGGGAGCTGTCGATTGGACATTTTCCACCAAGAAAAGCAGTTGCTCTTCAGTTTCAAAnATAGTTACACCTAGTTTTGATATTTATAATACCAGCAAGACAAAGCGTTCTAAACAGATA | C | G |
| 40 | AAGTGTGGGAAGCAGGCACAGACTCCCGTGCAGAGAGAAGGTGCCAAGAATGAGGCCATnGTGGAGGAAATGATGCACAGGTGACTGGGGAAGAAAAGGGAACAGGAAAGGTGAGAGGAA | C | T |
| 41 | TTTCTGTCCTCTCTTCAGAGATGACATATTTTTATTCTAGGCTCAAAGAAGTGGGGAAGnGTAAAGGGTAAAATAGGCTAACAAATTCTTGCTGCTGCTGCTGCTGCTGCTAAGTCACTT | G | T |
| 42 | TTTGCTAAAATATGAGCGAACAAGTCCAGTGTAGGGAGTTGAACAGAGCACTATGGTATnGAGTGTGTAGTTTCTTGCCTTACGATGCCTACTATGATTTTAAAGTTACTTAAAATTGTT | T | C |
| 43 | TGTGATCAGTCAGCAGACAGAGGACAGAGCTGCTGAGGTCTTGGGGGCCCAATGGGTACnTGGGTGCTTGTAACATTCCTGAGGATAGTAGGGACTCTTTTGATAACAGCCTTTCTCTGG | T | C |
| 44 | GTAAACAGTCTAACTGCATCCCAGATGCAGAACTCTCATATTCCGGTTATTACAGGCTGnCAGAGGAGCCCCTGATTCCTCTCCCAGCATCCTAGAAATGTACCTGATGTCCTCTGGTGA | T | G |
| 45 | ATAGAACCAATATTTCTCAGCTCAGGATATCATTGGTATAAAATCTCCAATTGCCTGAAnGCTGATTCTTCCTCGTGCTTCAAAAAGCTCGTCTCTGCTTCTCTCCTTTCCCCAAGGTGT | T | C |
| 46 | AATGAATACCCACAATCTTTCCTGAATAAGCCATCTTCTTCCCAACTGATTAGAAACAAnGCTTTTATAAGTACTCATTTTTATTTTGTATAATGATCAAATGTACCATCTATTCTCATT | C | T |
| 47 | CCACCCCCCAGGTGCGCACCCGGCTTCTTCGGGAACCCCCTGGTGCTGGGCAGCTCGTGnCAGCCCTGTGACTGTAGTGGGAACGGTGACCCCAACATGCTCTTCAGCGACTGCGACCCC | C | T |
| 48 | GAAGTGGACCCTCCAGGCTTAGGCAGGAGGATTCAGGAGGGGTTGTCTGCTCAGGGAATnGGGTGGTTGTTGGGAGGTGCAGAGGAAGGAAACCAATGTTTTTCTCCCTTTGCCCCCATA | G | A |
| 49 | AGGGCTACAGTCCATGGGGTCGCAAAGAGTTGGACACGACCGAGCCACTAACATTGTCAnTCACCAGTCAGGATGCACAGCACTTCTGGTCCATCTCTGTTCCCCTGCTTGCTCCCCCAG | A | G |
| 50 | TGCTGCGACTTGCTTAGCTTTTCCTCCTCTGTGCCCTTGATATTGCTGCCTGTGGTCACnGTGATTTTGCTGGGGTCCTGCACACAAACCAAAGAAAGGGAGACACTTGAGGGCAGGAGC | A | C |
| 51 | GCATGGCAGAGCAGAACAGTAGCAGTGCTTCCTCAGCCACTCGGCAACAAATGCCTCCTnCACCCCAAAGGAAGACTGACGAAAAGTTCTAAAATTAGAAGATGAGCTGGGGCTGAAAGG | G | T |
| 52 | TTTCTAAATAATTTAAAAGAAAAAGGAGACTAACTCCAGCAATGAAAAGGACAGGCAAAnGACATCAAAATAGAGGGAGAATGCTAATTAATACCAGTGTTTAGGAAGGCAACTGGGCAA | G | T |
| 53 | TTAGTCCCTCAAAATTTAGTTCATCATGCATTATCTCAGTGAAGCCCTTCCTTCTATATnCTGTCCTAACTCAACCCTTCAGAATAATCACCCTCTGTGAGCTTCAACAGACCTGAGACA | A | C |
| 54 | GGCAACACGGTATGGACGTTCGGGGTGAGACACAGACCTGTATGCTATACCAAGGGGCCnAGGCTGCTGCTTCCTGCTCAGAACATAACCCAAGGCTGTTCAGTCCAATGGGCCCTCTGC | G | A |
| 55 | TTGTGTGACCACAGGCACATCCTTCTCTTACAGTACAATCGCCCCTTTTACACAAGGAAnAGAATGGAAAGGAAATTTTCAATCAGCAAACTCTAAAGGAGACACAATTTGGGACTCTAT | G | A |
| 56 | AGGTGGCTCCAGGCTGTTCACAGTCTCCTGGGAGTGGTTACCTGCACCAGTAGGGTCTGnGAAGTGAGAACAGAGGACAGTCCTTCTTCCTCCAAATCTAAACCCTGAAAGCCTTTTGCA | C | T |
| 57 | TGGGGCCACTCCAGGTACCAGTATGGAAAGGTGAGGGGGGGTAGGTGGTGTGCCAGCACnATGCTGCGAGGGTGGTCCTCCTTCTGGAACACTGTTCAACATCCGGAGGATGCAATGGAT | G | A |
| 58 | GACAGCAACCTCCTGGGAGACTCTGAGCCAGAAGCCCCCACCCATACCAGCTGCAGAGTnCTAGAAAAAAGTGGATCTCCTGACAAAAAGTGACCAGATCCGCTGAGTACAGAGGAAGAG | C | T |
| 59 | CCTCAGGGAAGCCTTCCCCTTTAAAGCCACACAGTCGGTCACTCAGGCTTCCTGTCCGCnGTGGCCTGTGTTTGCATTAGTAGCGCTCTTATCCCAGGATCTCATAAGTAGCTGTTTAAA | A | C |
| 60 | CCGGGAGTCACTCAAAAGTAATGCCCAGAACTCCAGCCCAGGCTGTGCCTCTGAGTAAGnGAGGGCCCTCCTGGGCCTCAGTGGCCTTACCTGAGAAATGGGACTTGATTGTGTGTTTGT | C | T |
| 61 | TGGGGGCGAGGGACGCTGACTCCCAGTCCCGGATTACTAGGTCTAAAGTTGAGTTGACTnCACTTCTGATTCTGATCTCAAGGCGTAATCTGATTCTACAGAGTTGATATTCTACATGGA | A | G |
| 62 | CATTTATTCCCAGAATCCAGAGGGCTGAACCAACAAGCACCCCTCTTCACCCCATTTGAnGAAGGGACCAATCTAGCCTCCAGCTGGAGACATCACAGGTCCAGGGTTTGTGAGGCTGCA | G | A |
| 63 | GAGTCCCCCTTACCCCATGGACTTGGATCTCACCAAGGGCAGGACTGAGATGTCAGGAGnAAAAGAAAACTCATCTTCTTAATACAGTTATTGGTATAATCTGAAAATATGACTAATAAT | A | G |
| 64 | GCAGGCTGTCACCACCTGTGCCTACTGTGCCAGTGGGCTCGCAGCAGGGGCACAATAAAnGAATTGCCCCTCCTCACTGTGTGTCCCTGTTCCTCCCATGAAACTGTCAGGGCACCGCTG | C | T |
| 65 | ATCTCAGGCACTGTTTGTTCACACCATTCCTTCTGCCTGGAGTGACCTTCCTGTACACAnGCCCTTCAAATCCTGCTTCCCTTCAAACCCCGTTTGCCACAGGCTTGGTGAATGGCTCTC | C | T |
| 66 | GCAAGGTGAGGAGGGATCACTCAGAACCTTTCCCATTGCCACGCAGAGGGCATAGGCCCnAGAGGTAAGTAACCAGGCTTCAGATCAGAGCCAGACCAGGCAGAAACTCCAGGCCCTTGT | A | G |
| 67 | CCCAGCCTTAGTCAGTTCCCAAGCCCCCTCAGCAGCAAGCCTGCGGGCTGGAGATCTTGnGGTTTTCTCCCTGGTCTGTTGCTCTGACTGGGCTTCCACTGTTCAGCTGGAGGGTTCCAA | A | G |
| 68 | GCAAGTCTGTGCTACAAGGCTGCCTATCTGGGCTTGGGACAACTACCTCCTCCTCCTGCnAGGTTCTAGCTGAGCCTGGCCTCTCCCCATCTTTATACAGGGACCAGTTCTGTTCCAAAG | C | T |
| 69 | TGTCTGTACCATCAGCCCGGCAGTTGGGCAGCAGAGAACTCAGCTGCAGCGGCCAGCACnGTGGCTCTCTGCCCTTTCTCCCCCGAGACAGCCTTAAGTCCTTCCAAGAGTGGGGAGCGG | G | A |
| 70 | CATTGAAACTTTCATTAAAAAATAAAGGAGTGTGTACAATCCATTACTTCTGGGGCCCCnCTTCCTAGACCCCACGTGACTCCTGGCTCTGGCTTCAGACACCCTGCAGGCTGCAGCCTT | T | G |
| 71 | TTCCAGGGAACTTTTTGCCGAAATGTCGGGGTGCCCGGCTGCAGGCACAACATAAGATCnATTTCATTCTCACCCCAGGCACTCAGGGAGGCAGAAAGTCCACAGGCTACTTGGACACAT | A | G |
| 72 | CCCTCTGCTCCGTGGGCCTGGAAGCTCTCCTCCCTGGAATCTGGGGCCAGAGGGGAGAAnGCCCCTGGCCTGACATCCAAGGCCGCACAGTCAGACCCTATCTATCTTCCAAATCTCTGC | A | C |
| 73 | TTCAGGGAGTCAAACAGACTTGAGGTTGAATCCTGACTGCCATTTGCTGTGGCATTCTTnGTGCCCACAGATCCCAGTTGACTGCTTCCCACCTGAATTAGCCCATCAGTTGACCCTTTG | C | T |
| 74 | GAGCCTTCTCATAGGACGCAGAAGAAGCTGACACTGGATCTGTCCCGTCACCTGAACTCnTCGGTAATTTATTGATGCTGGATGCTGGCACCTAGCAGCTTGCCGGGTCAGCCTCAGGGA | G | A |
| 75 | GAGGAATCTGAAACCTGAGGCCATGAGACAGCACATCTGTCTCACCTGGCTGTGTTTCCnTGGCATTTGCCTGCCCTGACATGCTGGGACCCTAGGATGTGTGAGGATCCAGCTAAAGTA | T | C |
| 76 | AATAATCCATTTTTTTATAGAAATTCATTGTAAAACCAATTTTTTGGCTACTGGGGTGGnGAATTTGTAACTGGATCCCGCCTTCTAGGCGGGGTCACATCTGCCCGAGGTGGGATCTCA | A | G |
| 77 | GACAAAGGCAGAGATTTGTATCTGGAATGAGTCAGCAACTGAGTAGGGTCCAGAAGTGGnAAGAGGGAAAGGAACTTATGAAGGTGACATTGACCGGTTAGGGTGAGGCCAGTCAAGGGT | C | T |
| 78 | TATTTGGAGAGGGACTCTGGGCTGGCAGCCACATTTGGGACCCCCAGAGGGTCTGAGGAnTCTGAGGCCCAGGGAGGGGCTGGGACCCGCCAAGCTCTCCCCGTGAGTCAGGTGGCTGCT | A | G |
| 79 | GGTCACCATGAAGACAGGCAAGAGCCTGCTGCACCTCCACGGCACCCTGGAGATGTTTGnTGCACGGTGGCGGGGCAAGCCGCCTGTATCCCTCTTTGTCCTGGAAACTGTGAGTGGACT | C | T |
| 80 | TGCGGCCCTGGCAATCATAGAGGTGAGTTTCCCATGGTGGGGAGGGTAAGCCTTGATGGnCCTAAAGCTTGTCTCTGGAGAGGGGACTGCTCTGCCTTTGACAAGGTCTGTCGCTGCCCA | A | G |
| 81 | GACTGGATACCTGCCTGGGTGACCCTTTTCTGAACCTCAGTGATCTTGTCTGCAGCATGnGCATAGTCAGGAGACCTCTCAGGGATCAACAGAAGGGCCTGAGTCCCTTGAATGACATGG | A | G |
| 82 | CTGAATGAGACCTTTGCTTCTCCGCAGCCATCTCGGGCACCTTGCAGCAAAGTGATGCTnTTCGCTCAGCCCTGAGAGAGGTGCCCATGGGTAAAGCTGGTGGTGATGGTGGCGGGCCTC | T | C |
| 83 | TCCATTCCTGGAGGCACACAGGTGACCCAGAAGGCGAGAGGGACCCAGGGGGCTGGGACnCGTCCCTGGGGGTCTGAAAAGGGCCCTAAGGAATGCGGAGGGACAGATTGGTGAGAGGAT | G | T |
| 84 | GGGGGTCTCACAGGGAGCCCCCAGCGAGCAATGACCGCTGTATGATGGCAGTGACATTCnATTCACCTTCCCTTTCCTCCAGGACACAGAACTCTTGGCCATGTTTTCCGTGGAGAATGA | T | C |
| 85 | TTGGTTCCTCCTTCGTGGAATTCCTAAAGGATCTTGCCAGGTAGTGATGATGCTCATGCnGGCATAGCACCCACCGGGTACCAGGGGTGATTCTATGGGATTTACTTGAATTACCCTATT | G | A |
| 86 | GACTTCCTCTCCAGCCACAGGGCTGGGGTTCGGGGTGGGGGCTCAAAGATCCCTTCAGAnCTGAGCGAATCTCTGCCTTTTCATTCTTCCTCTGCAGGTTGGCCTCTTGGAGGTGCTGGT | C | T |
| 87 | GGTAAGGGGAGATTAGAGCTCTGTTCTGCCTTCCTCTGCAAACCCAGGAATTGGGAGGAnGAAGAGAAGGGAAGGGAAGGGCAGGACAATAGCAGCCTCTACCTTCAAAGGATGGGGCTG | A | G |
| 88 | CTTGAATGGGCAATGTGGGTCAGACGCCGGCTCTGGGCCATCCTTGGGGGCACCAGTCGnGGTCCCTTCTCTTGAGCGCCTGCAGGACCTCAGCACTACTCTGAGCCTCAGTTTCCTCAT | T | C |
| 89 | CTCCACTGTTCAGAGTTACTGCTGCCGAGAAATGCGACTGGCCTGACCCTAAGGCAGGCnGCTGTCTGCTGAGCTGTAGCCAGAAGCCACCCCATTCACTAACCAGCTCTGTGTTCTGCC | G | T |
| 90 | CATTCCTCAATTCTCCCACCCCAACTCCACAGCGCACGGGGAGGGTCAGCACTCAGAAGnAGCAAGTTCAAAGTCATTAGACTGACTGCACACTGCTGGAGGGCTGCCCTCTGGCCTAAC | T | G |
| 91 | CTGGCCATGTACGGTCTGCTCAGAGCAACTGCTCCCCAAGCCCCATCCCAAAGCTCCTTnTCTGCCAGACAGTGAGGAAAACGGCCAATCTCTTCATGTTGAAAACAATCAGACAGCATT | C | T |
| 92 | GAGTCCTTGCCAGGAGAGAAGAGGAGTCTTTCTTCTTCCTATTGTCATGGGATGTGAGAnTTCCCCATTCTGGCCTCCCAACTCTACTGAAGTTTCTGTTTAATTTGTGCACCTCTGATC | A | G |
| 93 | GACTTTGAGGAATATTGCTTTCGTTTACCATCCCCATTGGGCAGTCAGGGACACTGAGGnGGGAAAGAGCAAGCAGGTGCCAGGGTTAGGCTTGGACTCGGTTGGGCTTGACCTCTTTGT | T | C |
| 94 | GGGGGTCAGGGGCCTCTGACTTGCCTTGCCTCTGATTCCTCTCCACTGGGCATGGGCCCnGGGAGACCCCCAGTGAAGAGGAGCGAACAGTAACAGAGACATCCATCCTCGTGGTCACAA | C | A |
| 95 | TTGGAGTAAGGAGCAAATTCCCAGGGGGCGAAGGTTGCCTTGGGGAATCAGGTGAAAGCnGTGGTTTCTTCCCAGAACTTAGTGGATGTGCCCTTGGAGACATGAGCACACCAGAGAGCC | T | C |
| 96 | GGATGCCGAGGGGAGGGGCTGGGTGAGGATTGTTACACTGAGTGTCTGCAAACAGCAACnATAGTCCTAGCTGTCTTATTCCATTTGTTCTACTTATGAGGAATAATTCCTGCCTCCTTC | A | G |
| 97 | TCTGGGTTTCTCACCAAGACTGGGTCTCAGGCAGGAGGCAGGAAGGAGATGGGACCCCCnTCAAGCAGGAGGGGCAGGCTGTTGTCCCCCTGGGGAGCCAAGGAGGGCCTGACTCATCCA | A | G |
| 98 | TTCCCTCAGGGAAGTCACCCATGGGCAGGGTCCCTGGTCTATGTGCTATAATAACAGCAnATGCAACATGTCAGGAGACACGTTCATCAGCGACGATCACATCGTTATGACACAGGGGAC | A | G |
| 99 | GCCTCCCACCCATCACCACAATATAAGGGCCATCAGCACCCAGAATTCCAGGAGACTGCnCAGTGGAGCAAGGATTTTGTAAGTATTCTCCTCCAAACCTGTGAATGGTGGGCTTCCCCC | T | G |
| 100 | AGGTGGTTTGCAGGGTGCTTCCAGTGGATAATGATCAGTCCTCTTTCTCTCGTGTCTCTnCTCTCCCCGATCTCCATCCCAACATCATTCCTGTCTTCCTCTCTCCATCTCCCGCTGTGT | C | T |
| 101 | ATGAAGGCTGCCTCACAAGGTCAACTCACGTCAGGGAACCCTTGAGCCCCTGGGGTTATnTCCGGGGCCTTCAACCAGCTCTCCTCAGCTCTGCCGAGCGGACCTTAGGTCAGGCTGTGC | G | A |
| 102 | TGCTTCTTAAGCTGTGTACCACACTAAATGGAGACTGTTTTGCAGCTATCTTTCAGCAAnTGAAGACTGATTTCATGCTCCCCCAGGAATCCAATACTTCTGAGGAGGGCCAGGAGAAGG | T | C |
| 103 | CAGACTTCAGTACACCAAGCTTCCCTGTCCTTGTTCAGAGAGATCCCATATAATGTCCAnATAGAAGAGAGACTAAAGTGGGCAATGTGAAGGGACTTCTCCATCTCCATTCACTGTGAA | T | C |
| 104 | GTTCTGGCTGAGAGAGAACTAGTCTGGAGTTGAAGGCTGGGGCAAGTTGGCCTTAGCAGnTAACAAGGCTGAGTCATGAGCAGAGGTCAGTTCATCCTGCCCAACAGGTGTCTGATCACA | G | A |
| 105 | AAAGCCTGGATGCAGATTATCTTAAGAACCACATCACTAAGTCACAGCTTGGGGAAATGnTGAGGGGGCATCCTATGCATCCTTACAGGGGAGGGCATGCTGCTCTATGCCAGCTGCACC | A | G |
| 106 | CAGGAGTGACAACTGAGCCCAGTCAAAGGACACTCTCAGATACTCCTCCCTACAATCAGnACACACTATGCTCATCATCCTCCATCCCCAGCAATAAAGAGCCATTTCTGCATCCTGTGT | G | A |
| 107 | CAGCCACCTCTCCCCAGCCCCCAGCAGGTCCCGCTGTCCTGGCCTTGATCTCTCCCACCnAGGACTGATGAGTTTTCTCTCGGGGCCCTTCTCAAAGTGCAAGTCCTCAGTTTCCCCTTT | C | A |
| 108 | ACCCTGCTACCTTAGTCTCCCAATCTGTAAAATAAGGAGGCCTTTGCAGCTCTAGAATCnTCACCTTTAAAATGTTCTAAGAAACTTTTCCCATAATCAAGTCTCTCTTTAAAAAGAATC | T | C |
| 109 | TCCAAAACTAGCAACACATTTGTAGACCATGTCTCCCTCCTCCAATCTGATGCCTACTCnAGGGTCACACAACCACACTGGGAACAACAAATATCAAAGCCACAGGAGAGCTTAGAGGTC | A | G |
| 110 | CTGGGGACCCAAATGCTGGGCCCTTTCCTTGTGAGATCTTGGCCAGTCACCTCCCTTCCnTAAACCTCTTTCCTCAGCAGAAAATGGGGCTTTCTGAACCTCCTCACAGAGTTGTCCTGA | A | G |
| 111 | CCTCTCCCTCCTCACCTCCTGGCTTCTTTCTTTCCTTCCCTCTTTCCAATTCTCTCTGCnTGTGTCTCTCTCTCTTTTGCTGGCTCTGTATGACCCTCAGCTGTGCCCACGATTCCGCGA | G | A |
| 112 | TTAGGCTGGCTCTGAGAACAAGCCCAGTGGCTGGGTCTGCAGTTGGCTGTGGGTTTCACnAGACAGCTGTATGGAGCTGTTAGGGGTGCTTGGGGTCAGCTTGTAGCCCAGGACTCCTAC | A | G |
| 113 | GTCCCATCAGTGACCTCCTGGCAACCCTCACCTGCTCCCTCTCTTTCCATCCTCACACCnACTGGCCGGGCTCATAGTCCACTTTCTGAACCTCCTCCGGCTTTCCCTGACTCAGACCTC | T | C |
| 114 | GGGAACTCACTGTGAATGGAAGACACAAGAAGTGATTAGATATCGTTTGAAGAGTTTCAnTGCAAATGTTCCCAGCACTGGGCCTTGGTCATCATGAAAGGATGAGAGACCCCTTCAGTG | A | G |
| 115 | TGCTGGATTCCAAGGGAGTGAAGAATGGTGATAGAGTGGTAAAGTAGAAACATTGCCCCnAAAAGATCTGCATTCAAGTTCAATTCAACCCAATAAGAAGTTCCATGAGCATATGGACAT | A | C |
| 116 | CTGGAAGGTCATAGTCCAGACAGATGGGCTAAGGTCCTGCCCAAGGCCCAAGGAATCGCnACACAGAACACTTTTGGGTGTCCAGCAAAAGAGTAAAAAGTAAAGGCTAAATTTAAAGGG | A | G |
| 117 | GATCAGAGCAGGCATTATTAGTTTGCCAGGTGAGGGAATTCTACCTCGTAATATAAATAnTGTCTTGAACAGGACTATAACTTTTTTCTTTTAAAATATATTTCCTAAAAGCCAACCAGT | G | A |
| 118 | GAAGGTGGATTTTTAACCACTGGACTGCCAGGGAAGTCCCTCCTTTCTGCCATTCTGCAnCTATTGTTGCCTCCTTCCAAACCGGGTTGATGCTGTAAAGCCCAAAGCTGACCATGTGAG | C | T |
| 119 | AAAAAAAGAAAGCTAAGACGCAATCAAGAATGAGATTCCTAGGCCCCATATAAATATCAnTTCTTCCAGGGAGCACTCAGGTATTGAAGTTACTGGCTTCCTCCATTGTCCACCCCAACC | T | C |
| 120 | GAGGCCAAGAGAGGTGGTGGCATTGATTTGACCCAGTGGTCAGACTCTAGATGAGGCCCnGACAGGGAATTCTGTCTTTATTTCAGTTGCTCAGTCCTTTCTATGATAGAGAATGCTGTA | T | C |
| 121 | GGCGCTTTCTCCCCGTCTGGTGTCTCGTGCAGTGCACAGCTGGGGCTCATAGGTAGCCAnAACAGCTTTGGGTTTTTTAGAGACCTTGTTTATAAATCCTTCATTTCCTTCTTTGAGCCC | A | G |
| 122 | GACAACGTGGGTATCAGGCTTTAGAGCTGGAATGTGGAGGATCCAGACTGCCAGGCTAAnGCATCTCTGCACAGGACATTGGGCATTTGAATTTCTTATGTAAATATCTCCTGGTCACAG | C | T |
| 123 | TAGTATAGCCCCCTACCCTGCCACCTAAGGGTACTTCTGTCCTTGGACTCCTGGCCCATnTGGACTTTCTTCAGGCCCAGATTGACAGAAGTTCTAACAATAAAGTTCAGAAGAATGAGA | T | C |
| 124 | ATTTGAAACAGGATTCCATTCCAAGATAAAAGTTGTGATTATTAACTTATATGGTACTTnTTCTTGTGTAACTTTTAAATTGCCTTTAGAATTCTAAAGATATTTTAAGCAACTTGCAGC | C | T |
| 125 | TCAGTATTTGGTACAGCTAGTTTGGCTGAGAAACTAAACCAGAACAAAAATTAAGTGTTnCTTCTTCCAGCTTTTGGCATGTCAGAAACTGTGGCTATGAGCCAGAAGAGTAACAGGCTT | A | G |
| 126 | TTAAGGGTACACAAGTAGCCTTTACAAAATCCTTCTTTGTGCGACTACTTAACCTCCCCnAAGATCAAGAAGCAGGAATCAGAAGTTATGGATTCATTGAATGTTAAAATAGGTCTTTCC | A | G |
| 127 | TGAGATCGTGCTGTTTTGTAGCTCAAAGGGGAAAAACAGGCAGGAGATGGAGATGCGTGnGAATGGATCAGAGATTACAAATCCACCCCTGAAGACAGACCACATTTTGCTTGGTAGGAG | C | T |
| 128 | ATGGGCATTGAAGCTTGAGAACCACAGCACTAGATGATATGAATGGGGTAGGGTTATAGnAATCTGTATCTTTAGCATGCTTTCCTAAGGTATATTGCAGGAAACACTGACAACGGAGGA | G | A |
| 129 | ATAAGGATCTTCATTACTCTCAGCCGTGGCACTCTCTTTAATGGTGGATCATGTATATAnTCTCATAATTTCCCAGCTTTAAAAGAATAGAAAAGGTAAAATCAAAGTAGTTCAATTGCT | A | G |
| 130 | TATTTACACATTTACACCTTAAACTTTAAAATCATGCCAGTTCATTAGTCACACTTTCTnTGATTTGACTCTTAATTTATAGAAATAACATGAATAGCCATTTTGCTGTTCCAGTTTGAA | G | A |
| 131 | CCCAGAAGAATTTAAACTTCATTTTTCAGGCGTTAGGGAATCATTGAAAAGTATTGACAnGGTCACATCTGTTCACAATTCTAGTAGCTATGAGGAATTGGGCTGGAGAAGTGAGAGTAA | T | C |
| 132 | GCATATTATAACCTTAGAATGTATTAGCATCTGGGTTCTCAGGATTCTTTATCTTGTTCnTAAACATCTAAACTTACATATACTAATAGATCAGGTAAATATGTATGGTGAGATAAACAG | C | T |
| 133 | TTGGGGCATATGCTTTATTTGTGGAACCAGGGATGGCTCTGATATTAAATGAGAAGCACnTGATAGACCCTCATCATTGATCCAGGAAATAAAATCCCAGTTTATAGCCTATGTTGATGT | C | T |
| 134 | ATAACAAAGAAGCAGTTAAGTTACATACCCCCATTATATTTTCAGAAGTGAGATGGTACnATATGAAAAAAAAAATCTCTGAATGGAGATGTCAGTTTCCATCCTGCATCAGCTAGAGTC | A | G |
| 135 | AAGGCCTACCACCCACGGTGATAGGTGCATTTTCTGGTGCATCTGACCCTCGCAGTTCAnCTAAGATATGAAATGATCTTTTTTTCCTTTTTTGTTTAGTATTCTAAACGCATATGACCT | G | A |
| 136 | ATCACTCCCGTGCAGGCATGATCTCAGACTCGAGTAACTGCCCTGCAGGACAGTGGCACnGTGCTGGGACTATAGAACGGGGGCGCTGACCACGTCCAGGGGTGGAGGGAGACGTGAGGG | A | G |
| 137 | TGGTTGCACCAGGTAAGGGGTCTTCCCCACCCCTACTTGAGAGCAGACCCTCACTCCATnCTTGGTAGCTATCGGGAAGGCTTCGGGTGTGGGTGTGTCCATGGGTTCCACAGAAAGGCC | G | A |
| 138 | CTTTCAAGCTCCTTTGTAGTGTCTTGCTCCAAGTGTCTCATGTGTCAATCCCCCACCCCnACCACCTTTATCTTTCTCTAACCGAGAACGCCACAAAGCCAAGGCCGTGTGTTTTAGTGC | G | A |
| 139 | AGGGAGCATCATGTGCGAATCTCCCGCCAAATCCCACTCCAGCCAGCATGCACTTCCCTnATCCTGACCCTTCCTCAGATCACGCCTCTCAGCGCAAAACCCGTGTGGGACACACCCCCT | T | C |
| 140 | TTGCACCCCCAGAGCTGCCAGCTAGAAAGGATTGACCTGGACTAGTGCTGGTGATTACAnAGCGTGTGCTCCCCTGTCTCTGCTCACAGAGAAGCACCGGAGACCCAGCAGGAGAGCCTC | G | A |
| 141 | CCACTGTTGTTAGGGAAGCTCCCAGGTACAGGCGGATTGGCCAAAGGAGTAGGACTGACnAATACATTTCTTGGCAACTCTACATTTTGCAACAAGGTTGCATTTGTTTGAGAGGCAAGT | T | C |
| 142 | GCAGGGCCCGGGCGGGCCTGAGGGACGTCAGCTAGGAGAGAAGTGGGGGTTCACAGGAGnGGGAGGAATTGGGGGTGTTCCGTCGGGAAATGGAAGTTCCGTGAGGAGATGGAGGTTCCC | G | A |
| 143 | GATATTGCTGCAGAATCCTTGAGGCCCTTCTTTCTAGTTTCCCTACCATCTGTACTTGAnTAGAGCCTGTCTTTCCACATATGTAAAATGAAAGGAGTAGATTAGATCAGTGTTTTCCCA | C | A |
| 144 | CTGTGCTTTGCCCTTGCGTGTACCTAGTGCCTTTTCTGTGACCATCCACACCAGCACCAnAGACTAGCTGCATTTGCTCAAAGCACTATTGACACACTTCTTCTTAAACACATTAGTGCC | C | T |
| 145 | GTTGTTGGGGAGGGGCTGTAGTGAGCTAGTTTCTCAAACCACAAACAGATCCCAGGGGCnGACTGCCCTCTTCCCACTCAGTCTCCATCATGTTAATAATGATTGACATTTGTAGAATTC | T | C |
| 146 | GGAAAAATGTGTAATGATTTGTTGGTTACAGTTAGAGAAAAGATTGGCCCCATTGCCACnCCGGATTACATCCAGAATGCACCTGGATTGCCTAAAACTCGCTCAGGTATGTTCAGAGGC | C | A |
| 147 | TTTGGAAAAGAATACACATCCTAGGCCCTGCTAGAGATTCTGATTCAAGAGGTCTAAGCnGAGGGAAGAGCTGAACCTCTCTTTTTAAAATAAATATTCCTGGAATGGGAACCACTGCAA | G | A |
| 148 | AGACATTTATGTGGCTCCTTAATGGAGGAACTGATGATGGAGGGACTGATGGAAGATCAnGTTGAAACTCTTCCGTGAGCTCTGTAAGGTTCCTAGTGTTCCTAGTTCTTGGTCGTCTTT | A | G |
| 149 | GTTTGTGATTGAAATTCAGAGAAAGAATTTTGTGGTTACCAGGGTAAGAAAACATCAAGnGGGCGACTGAAGTTTTTTCTTCTCATGCTAACAAGATAAAGCTGATTAACTCTGTGATGT | T | C |
| 150 | CTCCGTCCCTCCGTTCAGCACTGGGGGCTGCCTAGCTTCCTGCACCATCCTGTCAGCACnCTCAACCCTGCCCTGGGCCAGCTTCTCTAGGTAAAGTCTGTGGCAAAAACAGAACATAAG | G | A |
| 151 | CTTGCCAAAGTATTATAGGTTGTCAGCGATCAAGGAGCTTGTTTTTAAGTTCCTGAGGAnACTGCAGGCCCCCAGTCAGGATCTTTCTATTCAAACTAATTGTGAACTGAACAATGAACT | C | T |
| 152 | CTCACTCGGAACATCTTACAATTAATTTTTCCTCTCCTAATAATTTCTCAAAACTCCATnTCCTTCAGGTCTAGACCCCAGAAGGCTCCTTGACTATTCCAGGCTGCCCCATGGCCCCAA | G | A |
| 153 | GTGTGTGTGTGTGTGTGTGTATGAGAATTCAATCTGTGTTGAGAAGATCTATGGCACAGnAAGAGGGAAGTTGAAGTCCAAATACCTGTTGCTTGTAATTCAGTGAAATCAATTAGTGGA | G | A |
| 154 | TCCGAAGAGGGAAGACTTTTCCTTAATTCGTATGGTAATTTGGTCCTTTTCAAGACCCTnACACAATTCTGCTTCTTATATGTGCGTAAGGCATTTTTCAATTGATGTGAAATTCTCCCA | G | A |
| 155 | TGGATCAGTAGCAAAAAATATAGTACCATTAATTTGACTTTTTATTAATGCGTCATGCCnGAAAATGGTCCTCAGTTATGCAGGGATTCCATTGCTGGCTTTCCTCTGCATGCCCCCATA | C | T |
| 156 | GAGGAGAATACCAATTTGATAAAAGCTGTGTGTGCTGAAAAAGCCCAGAAATTGCTGTTnAGCAGATTGGAGCCCGATCGTTCCATTATTCCTGGGCTCTGCGTGTCCTCAGCTGATTCA | T | C |
| 157 | CTGATGAGAAATTGCCTGGTGTGGGGGAGCTGCTGGATTGCCAGTGAGGAAGAGGTCCCnACTGGAACGGATATGTGGACCCTCAGAGGAGCCCCAGGAAGCCCTTCACTGCAGCAAACA | G | A |
| 158 | TCAGCCTTTTCCCCAAGCAAGGGGAGCCGCAGGCGGCCAGCAGGACTGGGAATTCAACTnGCTGCCACCGTGACAGCAGGCGTGTTGGGGGAAGTCTCTTGGTGACAAGTAAGTGGGCAG | T | C |
| 159 | GGCCCATGTTCCCAATGCTGTTGTGTCCCAGAGAGGAGGGAGAGGCCCTTGGTCCCATGnTGATGACCTCTACATTCCACACCCGAGCTGTGCAGCAGGGGACCTGGCCACCCCCAGACC | G | A |
| 160 | GGTCTCGCTAGGGGGATAAATCCACTCCTTTACCAGCTGGTGACCCCAGGATCGCTCTGnTACCTGCTGCTGTGGAGAAGGGGGGTGGGCAGAGGGGCTGCTGCTGCTCCAGGTCCCCCT | A | G |
| 161 | TCTGGGTGGCTTGGCAGGTCCTTGCCCCTCTCTGGGCCTCATTTTCCCACCTGAACAGCnCCAAGTTCTCCTAAGCTCTAAGCATCTAGAACACCTCCATCTGTATCCTCCCAAGTGCTG | G | A |
| 162 | CAGCCCCCTCCAGGCAGGTGCCCTTGGGACGTGAGGTGTGGAGGCTCTGGGAGCATCTGnAAGGTTAGAGCTGGAGCCAGGAGGGGGGCCTCGTTCAGAGCTATCAATTAGGGGGCTGGC | G | T |
| 163 | TGAATGACCACACGGGGAAGCTACCAGGGGCGCTGGACCTTGTCTAGACTAATGGTTCCnAGTTTTTTTTGGAGGTCACGATCCTCTTGAGAATTGGACTGGTTGATGAAAACTGGGTTA | C | T |
| 164 | ACATACTTGAGTCAGACACTCACTTTATAAAAACACTAATGGATTTAATCCTTACAACAnGGGCATAGATGTAAACAACAGCTGATTCCCTTTGCTGTATAGCAGAAACTAACCACATTA | A | C |
| 165 | GTCCCTCTTTTTCCTCTGGGTCCTCTGCCCCTTTTGCCAACACATCAGCCCTCCAGTGCnCAGAGCATGGTCTGAAGAGCCTGAGGCTGCAGAGATGAAGGCAGCCTCCGATTGGATGGG | A | G |
| 166 | CCTCCTTTCACCCTGATGGAAACCCATCGCTGCCCTTCCTTCCACCCGTGAGACCCACCnTACCATCTCCCATCCTCCTGTGGGACGGACTCACCCAGCCCTAGGAGGAGAAGTGCCATG | G | A |
| 167 | AGCCTGCAAGAGCAAGCCTGTTCTCAGGCCTCTCCTCCACTGGATGCTAGGAGCTCCTCnCCATCCGCTGAGACCAAGGAGACAGCCAGGATTGGCCTACACACCATGAGGGACTAACCA | C | T |
| 168 | AGATTTGGGTGCATTTTGTCATTGGACCCTGCTCCCACGTCGTTCAGAGTCAGACCATGnAGGAGGCTACAAAATGCTGACGGAATGGGTGAACAGGAGCAGCAGTCATCACATCTCATG | C | A |
| 169 | GAGTTCTGAACAGTCTTCCCCCACCCGGAACTTCTTTCCAGTCTGCTCAGGCTCTGCAAnCACAGGGAAGAGGCAATCTTGAATTTCAAGTGTATTTCATTGAACTCATCTAATGATTGG | G | A |
| 170 | GTGCAGGTGAACAAGTTACCTAGAGACGTGCCCATCGCTTAGTGCTCAGGAACTGGGACnGGACCCCACGATGGTTGTTACCCATGTGATGGACAAGATTGAACACAGAACAAGCCTGGA | G | A |
| 171 | GGATATATATCCAGCAGTGGAAGTAGTTCTCTTTTTTAGGTTTTTGTGCCTCATGCTCTnTTCTAAGAAAGGGGTCCCTGCTCAGCCCAGCACTCAAGAGTTGAGTCAGGTAGTTGGATT | C | T |
| 172 | ACAAAAAAACCCGAAACCTGTTTGCCAGAAACTTATAGCCACCTGACCCCCGGATGACGnGGCATCTGGCCATCAGGGCTGTGTTCTGTTGGACCTCTGAGACCCCGTCTACAGGGCTCA | T | C |
| 173 | GGGGAACAGATGGAACCCCAGCCTGGTGTTGCTTCTTTATCTCAGAGCCTTCGGAGGGAnGTCAGCTGGCCATCGCCCTTAAACCAGCTGGATCCTGTCTGATGGGGTTGCCTACCGTGG | G | A |
| 174 | TCTGTCACCCCAGAAAGCTCCTACGTTCCCTCTTCCAGGCAGCCCTTCTACCCCAAAGAnATGAGTGTGTGCTTAGTCACTTCAGTCGTGTTCAACTCTTTGGGACCCCATGGACTGTAC | T | C |
| 175 | GGCAGCAGCGAAGGCAAGGCCCCCTTGATCGGAGGCAGAACTTCTCCGTACAGTAGCAAnGGGACCTCGGTGTATTACACGGTCACCAGTGGAGACCCCCCACTCCTGAAGTTCAAGGCC | C | T |
| 176 | CTGCTCCCCACAGCCTTGCCCATGCCTAGAGCCAGCTGTGTGAGCACCCAGGGGCCAAGnTAGAACACCAGGTAATTCTGCTCCTGGAATAATCCAAATCTATTTTGCTCTTGGATGGAA | A | G |
| 177 | TTTCAGTCAGGGATAAACCATTCCCATCAAGTCCCAGGGAAACTTTCCCTGTGGATCTCnTTGGTGTCGAGGGACAAGCTGGGGCCAAGGTCATTGAGTTATTCACTCTGTAGTTTATTG | T | C |
| 178 | GCAGTACCCAAGGTGGCGGGGGCTGGTAGGGTGTAGCAGACACCTTCCTCCAGAGCCATnATCAACTGCCACCTTGCTTGCTAACTGGCAGTGCCCAGCTTCCTGGCTGTTTGCCTGTGA | A | G |
| 179 | TCAGTTGTATTGACCTGTGGACCTAGCTACTCCACGTGTGTATATCTGTCCCAAAGACAnATCTGATACTTAAATTGCTAATTTTGTAATAACAGCCCATGAAACTTAGAGAAAACAATA | A | G |
| 180 | AAGTTTAAAGAGGAAAAAGAAAAAGTCCAGTCCACTGTGTACTTTATGCTGGAACAGTAnAGAGGAAACGCCACCATATGTGCCTGGCCCCTCACTCCTGGGGCCAGTGAAAAACTGGTT | T | C |
| 181 | TATCTTCTGTCCTGTTGGCTCCATCACTGCCCTTGTCACTCTTTAAACCTCTTCCTGTCnGTGACCTGTATGATCCTGAGCCTTGCTTGGCGTCCAAGACCTTCCATGCTCTTGCACACT | T | C |
| 182 | GGCCTCGGAAGCTTAGGCGTGCACAGTGGTCCCGAGGGATTCAGGGATCCGCCCCCGACnTGGGCAGAAATTCTCTGTGACCTCGTTACAGTCTCAGCCTCCCCGGGCCTCGTTTTCCTC | C | T |
| 183 | AATGACCTCACATTTCCAAACCTCAGATCCCTCATTTGCAAAATAATTTTTAAAATATCnGCTGAAACAAGAACCATCTGCTATTTCCTTTGTCAGTAATTTTAACCCAGGGTCATCCTG | G | T |
| 184 | AATAGAACTTATACAGTAGAGGTAAAAAAAAAAAAATCACTAAAAAAGCCCATAGAATAnGGCTTTCCAGATGTCTAGTGGTATGACCATACAGAAAATTTCTAAAAAGGAACTTGAAAA | C | T |
| 185 | TAGAACCAGAGCTCAAAGCTGTCCTTTCTCGTGTGTTTCACTGTTTCTCCCCACCGATCnACGTAGCTGTCTAAATATAGAAACTTAGCTATAAAAGACTTAAAAACAGGGACAAATAGT | A | C |
| 186 | TGTGAGTTCCCAGGTGAGAGTAAGAGGGCGAAGAAGAGGAGGGAATGGGCTGAAGTGGAnAAGAGAGGAGGAGTGTGTGAGAGTGGTGAATGGGGGGGCGTGGCTCAGGTAGTATTTTCA | T | C |
| 187 | ACGTTGTACTCGATTCAGGTTCTCAGGGCACCCCTGGATCCTGGTTTCCAGGAGCAGACnTGGACGTGTTTAGGGGAGATACCTGGGAAGGAAAGAAGACAGGCCTGGTGATCCCATGGG | A | G |
| 188 | AAAATTATAAGAATAAAAATATGATTTTTTGAATAATTATTTTGTTATCCTTAATCTGTnTTACGTATAGCCAATGCCTATCCCCCCACCCCCTGCTCTGCCAGTTTTATATTATTTTCA | A | G |
| 189 | GTTGCAGGGAGGAGGGCTCTAGAACGGTCCAAACTGGGCTTTACAAGACTGTCAACTCGnGTGGTATTGGTGATTTGAATCAATTGGTTCTCAGCCCTCTACATCTTAGCAGGAAGGCCT | G | A |
| 190 | CACTGGGACAAGAGACCCACTTTTCCTGGCCAGGGAATCTTTGCATGCTAAAGATAGGAnTTTGGTTTGCACGGGCTTTATTGCTTTTCTGTCTCTGCAAATAAAGCCAAAAATAGTAGT | T | C |
| 191 | ATCCTGCACGAGGCACATGCTGCGCCTCTGTGTCGCTGCTGCACCTTTTTCCCAGTGACnTTTGCAGCTCACCCTGGAGGGAACAGGTTGGCACACCACTGGAAAAACAACCAGACAGGC | T | C |
| 192 | CAGCTGACAGTAATGGGGTACAGCAGATCCAGCCTCTAGGTTTCCTCCACCAGTGCTTCnAGAGATCACAGCTCTGAGCAGGGCGCCACCTCCTCTGACCCTTAACAGTTTCCCAGATCC | T | C |
| 193 | TGGGAGGATTTTGTCTATAATCCTTCTCTAAATGATCCATAGACTTTCTGAATATACACnAAAAGGGTTTAATCTCTTCCTCCCTGTTTTTTTCCCTGACCCTTTTTATCTAGCTTCTTT | G | A |
| 194 | AGACACAGGAAAGGCCCGCAGCCCAGTTAAGTCATGTCTGAGGCGAGACCTGAAACCCAnGTGGGATTTTAGCGAGTCAGGAGGGGTGGAGTGATCCAGGAGTGTGTCGGCATGATAAAG | G | A |
| 195 | TGTTCTGTAATTATTCAGAACCTCATTCAGCGCTCGGTCATTCCTGTTACTCATGTAGTnTCGGCGTAAAGACAGGGGTCAAGCCCCCTTTTAGGTGCTGTGGGTATGTTTCCTCTTCGT | A | C |
| 196 | AGTAAAGGTTCGTGGAAAGAAAGAAGACATGAGGATCCAGAGACTCAGACTGGAAGTCAnAGTATCAGCTTTGTTAAACTTCAGATTGTCTAGCATTCCCTCTATTTCCAGAAGTTCAAA | T | C |
| 197 | CAGTCGATGGTTCAAGCACTGGAGTTACTCTATGCTCTGGGAGGTACGTCTGTCTCTTAnTCTGTGTTTCTTACGTATTTATTAGTATGTGATATTAGGTTGTATTCACTCTAGGGCTCT | A | C |
| 198 | GCGGTTCTCATTGATTTTATTTTGTCGGGAAAGTTGTTTCTCAAATTCTGTGTACCTTGnGGGAGGGGGCACAATGTAGCATGTGCCAGCATTTTTTTTAAGCCTGAAGGAGTTTTCATC | A | G |
| 199 | TGTCCTCACTCTCGGTATAGCTGTGCAGAGAGAGGAATCCAGGTCCCATTCCACAGCCCnACAGAGAACAGATGGACTCCAGTACCTGGGGAGAAAGGCTTCCGTAGAGGGATGTATTTG | A | G |
| 200 | CATGTCCCGATGCCACTAGCGAAAGCTCATCAGCTCGAAACCCATCCCAGATCCTCGGTnGCAGAAATTCACAACCGTTTAGAGGCTCTAAGCTTCTGTAGCTCAGGAACATAACTTGTC | G | A |
| 201 | CACTTCCCATTTCATTTTACTTCTGTTACTCCTTTCTCTTAGTTTGCTTAATTGGTGTCnGTGGTTGGCAAAAGGCCTAACTGTAGGTGTGAGAGCAGGAGAAAAGATGTTTGCAAGATG | T | C |
| 202 | AGTGGCACCATTTGTGCAGAAACACCTGTATATATGGAGCGGGAGTGAATTTGCTGGATnCTTGCAGTAAAGACAGTGAGGCCTCCTTCCTGCTCGCCCTCTCTCCCTCCCTCTCTCTTG | A | G |
| 203 | TATGCAGCTGTATTGTGCTAACACCTAACTGATGGTAACTAGCAACCCTGCATGGACACnTAGATAGCTGCACCCACAGTGAAAGCATATCCTTCAGATATACACATTGAGTGGGTAGCT | T | G |
| 204 | CATTTAAAAAAACAGTAGGTGTTCAGTTAACCCTCTGAAGGTGTGAAGGCATCTTCCTAnTCCTTCCCTACCCTGCAACTCACCTTGGGTGGTGCACACACCCCAGCATCAGTGCACGGG | A | G |
| 205 | CCTCAGTCTTCAGAACAGTTTCTCAGTAGAGGGATGCTGGAAAAGAATGGTGTTTATTTnAGAGGATGTCGGTGAAGGCAGTGGCTCCCGGGCTTACCTGCACAAGCGGGATCATTTGTG | T | C |
| 206 | ACTGTGCTAACCACATTGTGTCTATTAACTTACTTGATCTTGATGAAAGCTCCGCAGGAnGGAATGGAAGTTTGAAAAGGTCACCGCTTTCCAGGGATCACACTCCCTGGTTTATAACAG | A | G |
| 207 | ACCCCCTGAGGCCCAAATGGAGAGCTAACACTTCTATGAAGCTACTCCCTTCCTGAAACnGGGTGGAAGCCCTTCTTCTGTGGAATCTCAGAGCACTTTCTGTACCTTGTATATTTCATT | A | G |
| 208 | ACCCACGGTTTCAAAGACATGTAGAGACGTGAAGACACATAGATATACATATATGAACGnGCATTTCAAAACACACAGATTCACATGGATATGTGGACAGAATACAGACACACCCCCATT | T | C |
| 209 | GACTAGGGTCAGGGTCTGTGGGTGGGGACCACCCTCCCCTTCAGAGCTCAGGCAGGCTTnTGATGTCCTTGAGTGCTAGGGGAGTAGGTAAAACTGAGTTTTCTCATTTTTAGCAACCCA | T | G |
| 210 | ATGCAACAGGAATCCTAGGCCATGGCCGAGGTGGTTATCCCTTCATGGAATTCCCTTCCnGCCCTGATGTGCATCTGTGGAACCCCTGGCATCTGGATGTAGATGTCACCTCTTCCCTGA | A | G |
| 211 | CTTGGGATGGGGCAATAAGCACAGAGACCTCAGCTCTGGTCTGGGCTCCATCCTGACTCnTTTCTAGTCTGGTTTGAAGCCAGATAAGTCTTATTTCCTCTCCCTGAGCATCTCAAGCAG | G | A |
| 212 | GCATGGGAGGCATTGCAGCTTAGAAATCTATCTTTGCTTTCCTTTTTGCTGATAAGAACnTGAACTCTCTCCTGTTGGTCCCTCATGGAATGATAAGTGGTGGATCCATTCCCTCACAAC | A | G |
| 213 | GCAAATGACAGGGGATGTTACACAGGTTAGTATAATCCTCAGTGTGAAAATGATTTGAAnTCCAGATCCCAGGCAGGTATTTGGGACTGTGGATTTCTTCCCATGCTCTCTTTTCTGCTC | C | T |
| 214 | GCTTTACACAGAAAAACAAAACAAAAACAGAAAATAGAGATAACAGCTTCTATGGGTACnTCAAGTTCTCTTAGAACTGCATCAAGGGGGAAAAAAATTCTCACCAAATATTTGAGTTAG | T | C |
| 215 | ACCCTGAATCAGGAGTTCATTTACACAGATGTCTAGAAACTCCCGCACATCTACACACAnTCACTCACATTCTTTCTAAATACATGTTTTATCTTCCTTGTTATCAATGCTCTCCTAACG | C | T |
| 216 | CCACGAACTAGGACTCTGATTTGTGTTGGGGCTTCCCTTGTCCCTCATTTCTAAGGAGCnTCCTGTCTGGCAGGGCCGGGTTTAGGATTTGGAAATCAATGTCAGAATGGAAGGGCCGAG | C | T |
| 217 | TCCTCGCAGAGGCAGGACGAAGCCTCTCCAGGCATTTCTGAGTTGCCCTTCAGGGCAGCnCTTTTGTGATGTGAATGCAATGATGAAAGGCCCCAGATCTGTGGCTCTGGGTGGAATACC | C | T |
| 218 | ATTTCCATGGGGAAGGGACTTCCACATACTCTCTGCAGTTGTCTGCAAGTCTAAGAAAGnATGGAGGGAGGGAAGGGCTGGGGCCTCAGGCTAGAGCATGGTGCTGATGAAGTTCCAGCC | G | T |
| 219 | AAGGCCACGACTAGCTGAGACCCACTGCTGCCCCAGGCTCCCACGTACCTGTGAAGTAGnGGAGAAGATGCCGGGGTTCCACAGAGCCACCACTCCTCGGCCCAGTATCTAATGAGCCCT | C | T |
| 220 | GCCAGAGGCAGGGTCGGGCCCCGCACGTCCCTGTCCGTCTCCTCCATCACTATTACAGTnACGGCTCTGGGAGAGCACTCAGGCGTGAAAAAGCTCCTTAGAAACACTGGGAACTGAGGT | G | T |
| 221 | AGAAAAACATGAAAATGATCTAAGTTTTAAAATTTAAGAGAATTAAGCCATGTTAAAAAnGGAATGAGCAGACATCAAGAAGCCTGCTATTCAGGCCCCAGTTTATGATAGCTGATGCAC | T | C |
| 222 | AACTTTTTTTTTTTTAACACACACTAAAATGTAGTAGGCTGTGCACTTAAGATACCTACnTTCACTATATAATAGACTTTAGCATAGAGGAAGAATAGTCAAGTCACGAAAGCAATGCTC | A | G |
| 223 | ACAGAGGGAAACTGCTGGCTTTATTGGCTATGCTCTAGAGAAGATGAACTGATCCACCAnACAATCACAAGTGCTAAAATTATCAAGAACAATTATGTAAAGTTTAAAAATAAAATCAAA | C | A |
| 224 | TCAGAAGCCACAGCTGGCTCGTCAGTGCGCGAGTTTCTTCGGCTTGGAGTTTTGGACTTnTCAATTATCTCTTTGGGCTCACTGCTAACAGAGGGAAGCACAAAGTCCAAAGAGGTGAGG | C | T |
| 225 | CCACCTGCAAGTGGCTCAGATGCTATGCCAACATGATTGCTTTTTGTGGTCGGCGCCGCnGAGATGAGTTCAGAGGGTACCAGAGCGGTTACTATTGACCGTACATTGGTGGGGAGAGTA | A | G |
| 226 | CGGTGGCTCCAACAATCCATAACACCTTGCAAGAAATTGTAGACTGGCTAATGAAAATGnTAGTCAGAGAACAAAGAAAGCTGGAGAATACCAAAACAAAGCTAATTAATTTTGTTGCAG | A | G |
| 227 | AGCTGGGCAGTTAGAAATCTGGCATCAGAGAGAGAGCTGAATTCTATGATGCTCTGTGCnGGGAGTGCAGCCTCTTCAATTCTCAAAGGTTGGAGGTCAATACCTGACTGAGAAGCAGGG | A | G |
| 228 | ATCCTTTAAACACTGAACAGTACCTGGTGTACATAACAGGCACTCATTAGCTATTTACTnACTACATGATGAAAAGTCATCTTATCATAACCTTACAAAGAATCATAGGAGTTTTTCCAT | T | G |
| 229 | TCAAGGAAACAAAATCACATCTGATGGCCTCAAGGGTTGTGTTTTTGAAATGAATCTTGnTGATCTGCAGAATGATGAAGCTGCCTTTAGACAATTCAAGCTAATTACGGAGGATGTTCA | T | C |
| 230 | GAAAAGGGAATGGCAGGGATATCCATACAGAGGAGACACCAATGCGAAAGACCTGTGACnGGAAGAAGAAATGTATATTCAAGGAACCAAAAGAATGCTGAAGAGGCTAGAACAGACAGC | G | A |
| 231 | TTTCTTCCTGAGTGAGACTGCAGTCTGGTCTTAAGAAAATATACATAAATGTACTAATAnTCAACAGGTTCAATATCAGTAAAGGAGCTGGTTTTTGGCAATTTTTGTTGCCTCTTTGTG | A | T |
| 232 | GCAAGTTAAAAGAGAGCCTAGCAAAACACAGTTACAGAACCTCCTGAATGATTAAAGAGnTTAAAGTATGTTGGAAGGTAAGAAGAAAATAAGTAAAATGAGTTCAAAATGGTAAACAAG | G | A |
| 233 | CCAACGTAGTGGGTTATTGTCAAGATTTAAGTGAGATAACATGTGGAACATACCTAGCAnAGTGCCAGGTCCATGAAAACCCTCAATAAAGCTTATTATCTCCTTATCACGATCATCACG | C | T |
| 234 | GAAATGCAGATCTTCTTGAACTCACCCCAGTGATATTTCCATGATGCTACTTTATCCTCnCAAACCTACCAAATCAATTAATTTTTTTCTATTTCTGCCACAAGAAATAAAGCTGACTTA | C | A |
| 235 | CGACTTAGCAGCAGCAGTAGCAACAGCAGTAGCAGCTATATTGCTATAATATTGTTACCnATTTGTGACTCTATTGTTACACTCCCCAGAAGTAAACACAATGTATAAGTTTTTGAAAAC | G | A |
| 236 | AAGAAACAGTTAACAGCAAAAAGTACTTCATATATCCATCACCCAGGAAACACTGTTACnTTTTCTGACTTTTTCCATGTTGTTCTATGTGCTAAGTCACTTCAGTCATGTCCGGCTCTT | C | A |
| 237 | TACAGATGCTCCCTCTGGGCTTCTGTTTCACCACAAACTCCTCATGTAACCTCTCCCTGnCTAGCCCCTCCTCAATCTCCTTTGCCTGCCGCTACCATCTTCCTTCTGCATGTTTTTTAT | C | A |
| 238 | ACCCATTGAGCCATTGGGGAAGCCATTAACAAGTGAGGTATAAGTTAAATATTTAGTTCnCCACTATAATTTCATTTTACTGCATGCTGTATCATTGATTTATGTATTGCATATTTGCTA | T | C |
| 239 | AAAATTTTTGGTCAACCCAATACATGAAAGAGAGGTTTAGATTCTTTATTTAGTTTTGAnTAGCAACATGTAATTTTAACTGAAGCAGTTTTAAGTATAAAATTGCAGAAGAAATTTTAC | C | T |
| 240 | ATAAACCTTTTTAGATTTCATCACTGAGATTCCACCAACAACTTGGCATTTAATAATGGnATGCTATGTACTCACTGCAGATGGTGAATGCAGCCATGAAATTAAAAGACACTTACTCCT | A | G |
| 241 | CCATCTCACGGGGACGGGTGCCATCAGCTCAATGGGATCAGTCCCTGGCCTTCCTGGCCnCGGGTGCAGGCTGCAGATTGCCACCATGACCGTCCTAAAATACGGTGCACAGGAGAACGT | T | G |
| 242 | AGCTGTTTTCCAGAATTTAACACATCGTTTAACCAGACATATTCTTAGCAGCACTCAAGnCGTTCCGTGGAGTGGGTGTATTTGCAAATTCTGGTCACGCAGAGCTGATTCACAACCACA | G | A |
| 243 | CTCATCTGGGTCCCTCTTTGGGCTCAGAGTGAGTCTGGGGAGAGCATTCCTCAGGGTGCnGAGTTGGGGGGAGCATCTCAGGGCTGCCCAGGCCAGGGTGGGACAGAGAGCCCACTGTGG | T | C |
| 244 | TGTGGGGGGCGCTGGGGGCTGACCAGAAACCCCCCTCCTGCTGGAACTCACTTTCCTCCnGTCTTGATCTCTACCAGCCTTGAACGAGAACAAAGTCCTTGTGCTGGACACCGACTACAA | T | C |
| 245 | TTTCTCTCTAGTTGCAGTATGAGAACTTCTCTCATTGCGGAGTGCAGTCTCTAGAATGCnTGAGCTCAGTAGCTGCGTCGTGCAGGCTTAGATGTGGGATCTTTGTTCCCTGACCAGGGA | A | G |
| 246 | TATTTGTTGAGGTGTTTTTCGTAGGAGCTGTTCAACCTGAGTCCGAGGTCGTGGATGACnCCATTCAGGTACTCCTCCGCGAGGTTCAGGACCTCCTGGGGTGTGCCGCCCACAAATGCA | G | T |
| 247 | CACCAGCGATAACAGGTAAGACTTGTGGAGCCCCTTTCTGCGTGCCGGACGCCGTGGCTnCTGCGGTCTGGGTCGTGAGCATGTCGTGGACAATTTTTTTCAATCTTTAAAGAGGCATAA | C | T |
| 248 | GAGACAGTCCCCTGCGTGGCTGTCTTCCCCTTATGTTTGCAGACTCCAAAGAGCTGAGCnGACTCATTGGAAAAGATCCTGATGCTGGAAAAGATTGATGGCAGGATGATAAGGGGGCAA | C | T |
| 249 | GAGGTTGAAATTGAGAATGTATAGGCTGAATATAACTCAGAGTATTTTTTTAACTTTTTnTTTTATATTGGGGTATAGTTGATTAACAGTGTTGTGATTGTCTCACGTGAACAACAAAGA | A | G |
| 250 | GAATCCCCCAGGAATGGGATTATTCTGCCCAGAGGGCATTTGTGCCCAAGATCTCCAGGnACCCGGCATGGCCAATGCACAAAAAAGCAGCCTGTGTTCCCTTCAGGCTCCCTCAAACAA | C | T |
| 251 | GCCATGGCTGACTTGAGCATATTGGGGGACAGTGTTTCCATTCACCAAAATACCGAAAAnCAGGTTCGTGGGGACAGGAGGAGATGTGAAGTTCAAACAGTATAGCCACACCGTGTTTGA | G | A |
| 252 | CCTTCAATTCTGGAGTAGCCACTGTTGGTTGTGTACCAACATCCATTCTTTCCCTCTAAnTTACTAACAGAACCTGTTTGTTCAGTGTGTGATGAGCCCAAAGTAAGAATTTTCACCTCT | C | T |
| 253 | CAAGAATACAGTTGTATAAGAATACATAATTTTACACGGGGTTTCCTACATTCACGATTnTCTTTGGGAGGGCCATTTATGTGGTCAGGTGTGCCATTCATTCATTCACTGTGCTGCAGT | G | T |
| 254 | TTGCCATTCCCTTCTCCCGGAAAATCCATTTGTGTACCCTAACTCAGGCACACGGAGGCnCCAGGCCTAAGAGGAGCTGGGGGTGGGCAGGAGAAGGCACTCAGGGTGGGAGCAGGTCCC | A | G |
| 255 | CAACCTCAGGTATTTGGAAAATTAAATCTTTTGTGCAAAGAGGCATGTGGTGGTGGTGGnGGTTTGGTTGCTAAGTCGTGTCCAACTCTTGCAACCCAGTGGACTGTAGCCTGCCAGGCT | C | T |
| 256 | CCACACGTCCATTCTCTACATCTGTGTCTCTATTCCCGCCCTGAAAATAGATAGACATTnCTCCAAGAAAGATAGACAGATGGCCGAGAGACACAGGAAAAGATGCTCAATAACACTAAT | T | G |
| 257 | CACTAAACGCTTTGTGCCATGAGTAAGTTATACAGACACCACAGCACATGTGGAATAAGnCCGGCTCCTCCTCTCGTGGAAATGTCAATATCAAGTACTGAGCTTGACCAAGCAACACAA | T | C |
| 258 | CACCCTGAAGCGGGTAGGGAGGAGTTCTATAGTGCTCAGTGAGCAGGGTGTGGTCAGCTnATGCACAGTTCTAGGATTGGTTGCCATTGAACTGAAGTTTCCAGCGTCATCAAACTTCTG | C | T |
| 259 | TAATAGAACAATCTTCTTACTAGTCTTTGCCACCAGGAGATAATGAATGAGGAAATCCAnTGTGGGGGGCATAATGTTAGACACCTGAGAAATGAAAGAAATGTTTCCTGCTGTATCAGT | C | T |
| 260 | CTGTAGCACAAATCCCACCACCTTTTCCAGTGAGCTAACCCTGAATTCTTCACTCTGCCnTCCTTGGCTTTCCCCAGGAATGCCCATAAAGTCATGGCCTAACTTTCCCCTCACTGTTGT | A | G |
| 261 | GGAAACCCTTCAGAGCAGAGTCCTCGAAGGCATGGATTTCAGCTCTGAGGACAGGCACGnGCAAAGTGGGGTTTTAATTCCAGTGAGAACTTCCCCAAAGCAACACGTGCTGACTCCTGA | C | T |
| 262 | GCATATAAGGACTATGCAGACTTCACGGTGCTGGTGGTAGTGACAAGAACATTCAGATGnTGGCCCTTTCATAAGCCAACTCAGCTCTAAATGCTGACATTTCCAAGAATCATCCCCTTG | C | T |
| 263 | CATCATTGTAAGCACAGAAAGCAAATATTAAACAAAGGGAACCTACAGAAACAATCAGAnATCCTCTCCCTTTGTGATATCCATCACTTCAGAATAAATATTAATAAACAAACCTAAGGT | G | T |
| 264 | CAGCAGATTAGATGATGAGAGGAGAGAAGTTGGGGTACTGATACCCTGGCTTCTTCCTGnTGGTGATGGTGGCTGAGCTTCCTGCCCTGTGAGCATGGCTCCTGTGGTCCAGCCCTGTCC | C | A |
| 265 | TGAAACTTCTCTACTCAAACTTCATGCTGGTGCCTCTGTGGCAACTTTGGCCACCAAGGnTGGCTGAGGGGTAGGGAAGCTGGCATGGATTTAAAGGCCTCGAGACTAGGGGTGACCCCA | C | T |
| 266 | CGTGAGCCTCAGGGTAAGGTGGACATCTTATTCCACTTCATTCCACTCCCCCCCAGTTCnTGGATGGAAGGGAAGACCCATAATGGGTGTGAACATCAGTCTACAAAGTCATTATTTTCT | G | A |
| 267 | TGCACACACATACAGAAACTATTAACCAGTGAAGTTTCAGCTGCCAAACTGGGAATGATnTGCACAATACATTCCTGGATGTGATGGAGCTACACCCTGTGAGTATTTTGATCGGCAAGA | G | A |
| 268 | ATCTGAAGGCTTGACCAGGGCTGGAGGGTCCATTTTCAAGTTGGCTCTTCCATGCGACTnGCCAAATAGTGCTGGCTGTTGGCAGGAGACCTCAGCTCCTCGCCTTGTAGACCTGCCCAG | A | G |
| 269 | CAAAGACTGCCCATATGAAGGATTCCACACAGGACAGAAATGGCTGACCCTGTGCCCCCnCCACACTCAGGCACTGCCTGGGGGCTGCCTCTGCTGAAAAGCAGAGATGGAGACTAGATG | A | G |
| 270 | GCAGGTGGAGGCAGCAGGATGGGGAAAGGGAAACAAGGCTGGAGGGCTGAGTCTAGAAAnAAGTCTGGGGTCAAGGGGAAGGACCGGGGTGGCAGTGGAGAGTCTCAATTTGCTGTGCTA | T | C |
| 271 | TCTCTGTGGTTTTTCTACTCTCTTCTCTTCCTCAGAGTCCCCAGCACAGTTCTCTCAGCnGCATTGACCACTGGAGCCTGAGATGTTGGCAGATAGCCCTTTGCCAGCAGGTAGGATCCG | A | G |
| 272 | AGTGGGTGAGAGGGTTTGCCTGCAGGGGCGGCCTGGCCGCTGGGCTCTGGGGTTTTCTGnGCCCTCCTGAGGCGGCTGTGTTTGGCACTGACCATCGACGGAACTTATTTTCAGCAGGTT | T | C |
| 273 | TAGGGGCCCACCCACTCTCTACAGTGTTGACTAACCTGCTTAAGTGAAGTTGCTCAGTCnTGTCCGACTCTTCTCAACCCCATGGACTGCAGCCCACCAGGCTCCCCCATGGGATTTTCC | A | G |
| 274 | AGGTTATAGCCCGGCCTCTTCAGGGAGATGGTTGTTGATGTTTATTATCATCATTTTATnTCCATTTTACAGAAAACACACATGAGACTCGGGGCATTTAAAGAACTTGCCTGTGGTTAC | C | A |
| 275 | TGATGACAAAGGGATCTGGCTTTTAGGGTTTAAGAGTGCAGCATGATCCTCACAATCCTnGGGGCTTGGGATGAGGCCTTTGGACTCAGTGGAAGGGTGAGGACCTCAGGGACCCAGGTC | T | C |
| 276 | CAGGTGTAGCGGTACCATGCGTCCTTCTGGGCTGGCAACCCTAGAGCAGGGTCCAAGAAnCCTAAGCACAGGCTGCTGCCATGTCCCCTCCCTACCCATGTTCAAGGGCTCTCAGAGTAC | C | T |
| 277 | GATCCATGCTGCCCTGCGAACAGGGGAGAAGCACAACTTTCTTATTTGCAGGCTGCTGCnTTATATGTTTACCTGGCCCCAGAGAGGGCAAGTTGGTCCAGGCACTGAGATACCAGAGCA | A | G |
| 278 | CACTGCGCCTGTCCTCCATGCTGTTGACACCCTCACCTGCTGCATGATGGCGCCATTTTnATTGGACCCCAGGCCTTTTAACACCTGAGAGACCCTCTGCCTGGAGCCAGGAAACTGAGG | C | T |
| 279 | ATTCACAGTGATGCAATGACGATGATGCTTTAGCCTGAACCACAAATCACAGGTGACAAnGCAACATTAGCTAAAAATATATCTAATTTTTTCTGGGTTAGATTTACACCTATCGCATTT | C | T |
| 280 | TCTTGATGATAGCTCTTCTTACTGAATATGAGGTAAGATACCTCACTGTGGTTTCGTTTnGCATTTCTCTAATAATCAGTGATATGGAGTATCTTTTTATGTGTCTTTGGCCACCTGTGT | C | T |
| 281 | GTCTTTCCACTGTTTATAAGCAAATATACCCCCTATTTTTAGGTAACTGGTAGCATACTnTACTGGAGAAGGAAATGGCAACCCATTCCAGTGTTCTCGCCTGGAGAATCCTGTGGACAG | A | C |
| 282 | ATACTAGAACAGAAACTTCAGTGACCACACATGATGAGTAATATCATCTTTGCAAAAATnGTTTGGAAAAGTCACTAAGCAAATGGATGACTATATTCCTCAACAAGCAACAACAGCAAA | A | G |
| 283 | CACACTTTCTGTGCACTCTCTCACAGGTGCTGCCCTGAGACTCTTGGCCCTCTGTTCTCnCCCCACCGCCCTGTCCTAGACCATTGGACCCACGCCATCAGACTTCACTTACCACAAGAT | C | T |
| 284 | AAAGAAAAAACTAGAACATTTTCATGAGCCTGGGTCCTGCCACTTTGGGCATGTTTGCCnGTCATTTGCCCCGCCACCCCACACACCTTTAACCACCTACCTCACAGACACCCAGCGGCC | C | T |
| 285 | TCCTTTGTCACTCAGCCGGCTAGTCACTGCTAACTGGATGACTCTTCTCTTTCCAGGCCnCCAGTCCTCGCTCTGTGGAGGCAGAGCCTGGGTCTCACCTTTACTGTCTTGTCCCCAGCT | C | T |
| 286 | CAAACACAATCCCACCCGATGCGGTTTTGATTCTTGGCGCAGAATAAGCGCCCTCGGCGnTGGGATTTATGCTTCACTCGCAGTCCTCCGACCTCGGACTCTGCCCAGATGCCGGGAACT | T | G |
| 287 | CCGCCGCAAACCAAACTCCGCACCCTTTAACCCTGTGTACCACCAAGTTGGGCTCTGGGnGTGATGTTGGGGGGAGAGTGGACAAGGAGAGAGGGGTGGGAGGCAGCACAGTCCCTCCAT | T | C |
| 288 | AAATATTTCGTATTAGTGGCTGCTTCTCTTTTCTAAATGAAGGCGGCCCCTTCTGAGGGnGTAAGAGGAGGGTTGGAGTGAAGGTGAATACCTGCCAGGTCCTTCAGGGGAGAGCCAACC | T | G |
| 289 | TCTGGCTCCAAGAGACATGTCCTGGCCCCTACTTCCTCACACACTTGGTGTTGGCTTGGnGTGCTACAGGTGTTGTACCTGGGCTGGTGGGAGGGATGGGTAGATATTTCTTCTTGGGTA | C | T |
| 290 | GGGCATAGAGAGAAAACAAATGCCTCTTTTTTGTTGTTTTTATTAATGAGCCTCCCTACnCAAATTTTTCTTTTTGTATTTTACTGCCAGGCTGGCAATTACAAGAAGACATTTGTCTCC | G | A |
| 291 | GACGCCACAGATGTCCTTCTCCCCAAGGGAACAGTGGGCAATTGGAAGCGCCCCCAGGAnGGTAGGAATGTTATTCTGACAAAATCGCTCTTTCTGCAGGAGGAGCTGTGCCAGTCGATA | T | C |
| 292 | TGGAGAACCACTAAACATTTCCACAGTGGCTGAGCACTTTCCACTCCCAGAAGGTGCACnAGGGCCCCCGTGTCTCACGTCCTCACCAGCAGCAACTGCTTTCTGTCTTTTTGACTCTAG | G | A |
| 293 | AAAGGCCTCAGATACTGCAGCTGTTGGAGGAGAGGTGGGTGCCCTCTGGTGCTCTGCATnCTGTGCCGGGACTTGTCTGGGGTGCAGTCTTGGCCAGTGGGGAGCCTCCCCTCCCTCCCT | T | C |
| 294 | TGAAGGGCCTCGAAGGGAGAATACGCAGCCCAGGACGCTGTCTGGCCCAGTGAACACGAnGGGGGCCACTGTGCCTTGGCAGAGGCAAACATGTGAGGAGTGTCTTTAGGGGCTGGTGAG | G | T |
| 295 | GACACCACTTCCAAGGGATCAGCTAGCTTCCTTTGCCAACATTGGGACAGAGGGACATCnTGAGCCAATGGACAGGAGAGGGATAGCCTCACTCCTGACTCCCAAGTATGAGATTTTGGA | G | A |
| 296 | GCTGTGCGACTTTCCAAAGGGGTCGTCATGTCAGCCGCCTGGGGCCTGTGCAGCCCCATnTGCGAATTCCCACTCCAGGTCTCCCACCAGTCCTGGTCCTGTGCCCTTGAGAGACCCTGA | G | A |
| 297 | ATAATTGGAGCCTGTGATGTTTTCTTAATCCAGTACACAGCTGGGATTCTTTAGAGTGTnGGTTATCAGGATCTTAACATCTGACCATTTTGGAAGACTTTATTTTGGAGGGCAAGTAAT | T | C |
| 298 | CCAGTTCTAACTGTTGCTTCTTGACCTGCATAGTCCTGTAAAGCAAATAGTCCAAGTCCnCTCCTGTTGCCTCTTGACATGCAAGTCAGATAAAGCAGAAGAGAGGAAACCTTTTTATAG | G | A |
| 299 | CGCAATCCATGTTGCCAGTCAAATCTAAGTAGAAACAAGAAGACACCCTTTGAACCTACnCAAATTATTATATTGCCAGGAAAGGAAGAATACTCAACAAGAGTTAACAAGTTCTGGAGG | G | A |
| 300 | GAAAGAAGAAATGCAGGATCACTGAGGCCTAGGCACCCTGGGCTGGGCCTCACCTGGGCnGGTCTGCTGGAACCCGATAGGCTTTGTTCCAGACTCCTCTCATGTGTGTGCGTGTGCACA | G | A |
| 301 | TCGTGCAAAGACAGTTGTTGCCGGTATTTAGGGAGGGCTGCCCACACTTAGAGCGTCTGnTATAGCTCCAGGGTCCTTGGCCTGGGCCTGGCCACGCGCATCAGGGCTGCTGTGCCGCTG | C | A |
| 302 | GCTGGCCATGTGGATCTTTGTTGCAGGGCTTTCTGAGCTGCTGTTCTCAGACCCTTGAGnGGGCCAGATGGAGGAGGGAGTTCATGAAGCCAGGGTTGGGAAGCAGCTGGGTCTCCAGCG | C | T |
| 303 | CTGGCACGTAGACTCCAGTCCACACCTCATCTTTGCCAGCTCCCTGTCTCCGTGCAGGAnCTCCCAGGGGGCCTGGAGTCCAGGCCCACAAACTGAGGCTAGGACAGCCCACCTTGAATG | C | T |
| 304 | GTTTCTCCCTGGTCACAAGAAGTTCTACGCAACTGGCCCAGCATGGAGGAAGTGTTAGCnTAGCTCGGGGTGTTTGAGAGAGAGGAGGTCCTGGGGGTTACAGAGCGGGGCTGGGAAATG | A | G |
| 305 | GAGAGGGATGATTCTGGGGTAATCGCTCTTCCTGTCTCTGGGTGAGCTCAGCTCCAGGAnGCATTGGGACCAGCCGGCAGCGGCAGGGGTGGCCAGGGAGTGTGGACCTCCGGTCGCCAC | A | G |
| 306 | CTGAGCTGTTCCCCTGCTTTGAACCAACTGTCACCATCTCATCTCAGCTCATTTTTAGGnATCAAGTCTACAACTGTGCCACAGGACCTCTAGGCAATTTGGGAAGAAAAACCAGAATGG | T | C |
| 307 | CTTCCACTCTCTCTCAGTCAGTTCTCATTGTCTGCAGGGGGCATGTTCTCTCAAGTCGCnAGGACCCCTGAATTAGCCAGGCCTGGACCACTGCTCCCCAGCAAAGTGCTGCTCAGGGTT | T | C |
| 308 | TGGCGAACGACTTTGCGAACGATCTGGAAAAGTGAGAGGGAGGGTAGGAGGGGGCTTGGnGAAGCCGTCGAGGAAGAGCTTGAAAGGGGGCAGGTCCCAGAGGGTGGTGCAGTGGGTGCG | A | G |
| 309 | CCGCCCCAGCTGAGCTCAGGCTGTATCAGGCAAAGTAAGGACCGCCACGTCTGGAACTGnAGCAGGTACCCGAACCGTCCCATCTGAGCCGGGCTTACCCTGCTGTTAGGGTAGTAAGAA | T | C |
| 310 | TAAAGGGCGTTTTGAACAACGCTGCCCCCACCCCCACCACCGAGAGCCCGCCCCTATGCnAGCCAGAGGCAGCTTTAGCTGTGCCCAGGAAAAGGGTCCCATTTAGGACTCGGGAAAAAA | C | T |
| 311 | TGACCGCACACCGTCCTTTGGCTTTATTCTTTGTGTGTGCATACGTGCTCCGTTGTGTCnGACTCTTTGCAGCCCCAAGGACTGCGGCCTGCCAGGCTCCTCTGTCCATGGAATTCTCC | T | C |
| 312 | GTCCTTTGGCTTTATTCTTTGTGTGTGCATACGTGCTCCGTTGTGTCCGACTCTTTGCAnCCCCAAGGACTGCGGCCTGCCAGGCTCCTCTGTCCATGGAATTCTCCAGGCAAGAAGAC | C | G |
| 313 | ATTCTTTGTGTGTGCATACGTGCTCCGTTGTGTCCGACTCTTTGCAGCCCCAAGGACTGnGGCCTGCCAGGCTCCTCTGTCCATGGAATTCTCCAGGCAAGAAGACTGGAGTGGGTTGC | C | T |
| 314 | CCCACATCTCTTTCATCTCCTACATTGGTAGGCAGATTCTTTAGCACTGAGCCGCCTGGnAAGCTGAAGAAAACTACACCAGGACATGAATAGCAAATTATTAAAAACCAGCAACAGAG | A | G |
| 315 | AGCACTGAGCCGCCTGGGAAGCTGAAGAAAACTACACCAGGACATGAATAGCAAATTATnAAAACCAGCAACAGAGAAAAAATTCAGGGGAAAAAAAGGAACAAAGTTAAGGATGGCAG | TA | T |
| 316 | TTTTAAGAATGATTTTTGCTGGAAATTGTGGTTTTATACATTCCATTAGGTTAAAGAAAnCCTTCTGTTTCTATTTAAATAAAAGGTTTTGTTTTGGCTTGCTTTTAACCAAGAATTGC | GC | G |
| 317 | AAATTGCATTAATACGTTTTATAACATCAGATGATCCTTGCATTGTTGGGGAAAAAACAnTCACGATGGATTGTTATCCAGTGCTGATTTGATTTTCTACTCACGCCCTGGGGTGCATT | A | G |
| 318 | TGCATTAATACGTTTTATAACATCAGATGATCCTTGCATTGTTGGGGAAAAAACAGTCAnGATGGATTGTTATCCAGTGCTGATTTGATTTTCTACTCACGCCCTGGGGTGCATTTACC | T | C |
| 319 | GTGACAGAGCTCGAGTCAAACATATTAGAAGTAATATCATATGGATGTCAATTTAAAGAnGTGCCATATTCTTTTTTACCAGCCTCTGGTTCTTTCCACAGAAGGAATTACTGCAGGAG | C | T |
| 320 | TCAAACATATTAGAAGTAATATCATATGGATGTCAATTTAAAGATGTGCCATATTCTTTnTTACCAGCCTCTGGTTCTTTCCACAGAAGGAATTACTGCAGGAGCTATTAAACTAAAGA | A | T |
| 321 | TAAAGATGTGCCATATTCTTTTTTACCAGCCTCTGGTTCTTTCCACAGAAGGAATTACTnCAGGAGCTATTAAACTAAAGACACCGCTACTGCTATATAAATGTTCCGTAGTTATTAAA | T | G |
| 322 | TAAACTAAAGACACCGCTACTGCTATATAAATGTTCCGTAGTTATTAAAATAATTATACnTTTAATAGTTGCATTATTCAGTTCAGTTCAGTTCAGTCGCTCAGTCATATCCAACTCTT | T | G |
| 323 | TTATACGTTTAATAGTTGCATTATTCAGTTCAGTTCAGTTCAGTCGCTCAGTCATATCCnACTCTTTGCAACCCCATGGACCGCAGCATGCCAGGCCTCCCTGTCCATCACCAACTCCC | C | A |
| 324 | ACTCTTTGCAACCCCATGGACCGCAGCATGCCAGGCCTCCCTGTCCATCACCAACTCCCnGAGTTTATCCAAGCTCATGTCCATTGAGTCGGTGATGCCATCCAACCATCTCATCTTCT | A | G |
| 325 | AGTTCTTTGCATCAGGTGGCCAGAGTATTGGAGTTTCAGCTTCAACATCAGTCCCTCCAnTGAATATTCAGGGCTTGTTTCCTTTAGGATGGACTGGTTAGATCTCCTTGCAGTCCAAA | G | A |
| 326 | AGGTGGCCAGAGTATTGGAGTTTCAGCTTCAACATCAGTCCCTCCAATGAATATTCAGGnCTTGTTTCCTTTAGGATGGACTGGTTAGATCTCCTTGCAGTCCAAAGGACTCTCAAGAG | A | G |
| 327 | GGTTAGATCTCCTTGCAGTCCAAAGGACTCTCAAGAGTCTTCTCCAACACCACAGTTTCnAAAGCATCAATTCTTCAGCACTTAGCTTTCTTCACAGTCCAACTCTCACATCCATACAT | C | A |
| 328 | ACACCACAGTTTCAAAAGCATCAATTCTTCAGCACTTAGCTTTCTTCACAGTCCAACTCnCACATCCATACATGACTACTGGAAAAACCATAGCCTTGACTAGATGGACCTTTGTTGAC | G | T |
| 329 | TCAATTCTTCAGCACTTAGCTTTCTTCACAGTCCAACTCTCACATCCATACATGACTACnGGAAAAACCATAGCCTTGACTAGATGGACCTTTGTTGACAAAGTATTGTCTCTGTGTTT | A | T |
| 330 | CTACTGGAAAAACCATAGCCTTGACTAGATGGACCTTTGTTGACAAAGTATTGTCTCTGnGTTTTTAATATGCTGTCTAAGTTGGTCATAACTTTTCTTCCAAGGAGTAAGTGTCTTTT | C | T |
| 331 | ATTGTCTCTGTGTTTTTAATATGCTGTCTAAGTTGGTCATAACTTTTCTTCCAAGGAGTnAGTGTCTTTTAATTTTATGGCTGCAGTCACCATCTGCAGTGATTTTAGAGCCCCCCAAA | G | A |
| 332 | GTCTCTGTGTTTTTAATATGCTGTCTAAGTTGGTCATAACTTTTCTTCCAAGGAGTAAGnGTCTTTTAATTTTATGGCTGCAGTCACCATCTGCAGTGATTTTAGAGCCCCCCAAAATA | C | T |
| 333 | AGTAAGTGTCTTTTAATTTTATGGCTGCAGTCACCATCTGCAGTGATTTTAGAGCCCCCnAAAATAAAGTCAGCCACTGTTTCCACTGTTTCCCCAACTATTTGCCGTGAAGTGATGGG | A | C |
| 334 | TAGAGCCCCCCAAAATAAAGTCAGCCACTGTTTCCACTGTTTCCCCAACTATTTGCCGTnAAGTGATGGGACCGGATGCCATGATCTTAGTTTCCTGAATGTTGAGCTTTAAGCCAACT | C | G |
| 335 | GATGGGACCGGATGCCATGATCTTAGTTTCCTGAATGTTGAGCTTTAAGCCAACTTTTTnACTCTCCTCTTTCACTTTCATCAAGAGGCTCTTTAGTTCCTCTTCACTTTCTGCCATAA | G | C |
| 336 | ATGGGACCGGATGCCATGATCTTAGTTTCCTGAATGTTGAGCTTTAAGCCAACTTTTTCnCTCTCCTCTTTCACTTTCATCAAGAGGCTCTTTAGTTCCTCTTCACTTTCTGCCATAAG | G | A |
| 337 | ACCGGATGCCATGATCTTAGTTTCCTGAATGTTGAGCTTTAAGCCAACTTTTTCACTCTnCTCTTTCACTTTCATCAAGAGGCTCTTTAGTTCCTCTTCACTTTCTGCCATAAGTGTGG | A | C |
| 338 | ATCTTAGTTTCCTGAATGTTGAGCTTTAAGCCAACTTTTTCACTCTCCTCTTTCACTTTnATCAAGAGGCTCTTTAGTTCCTCTTCACTTTCTGCCATAAGTGTGGTGTCATCTGCATA | T | C |
| 339 | CTTAGTTTCCTGAATGTTGAGCTTTAAGCCAACTTTTTCACTCTCCTCTTTCACTTTCAnCAAGAGGCTCTTTAGTTCCTCTTCACTTTCTGCCATAAGTGTGGTGTCATCTGCATATC | G | T |
| 340 | AGCTTTAAGCCAACTTTTTCACTCTCCTCTTTCACTTTCATCAAGAGGCTCTTTAGTTCnTCTTCACTTTCTGCCATAAGTGTGGTGTCATCTGCATATCTGAGGTTATTGATATTTCT | T | C |
| 341 | CTCTCCTCTTTCACTTTCATCAAGAGGCTCTTTAGTTCCTCTTCACTTTCTGCCATAAGnGTGGTGTCATCTGCATATCTGAGGTTATTGATATTTCTCCCGGCAATCTTGATTCTAGC | G | T |
| 342 | TCTCCTCTTTCACTTTCATCAAGAGGCTCTTTAGTTCCTCTTCACTTTCTGCCATAAGTnTGGTGTCATCTGCATATCTGAGGTTATTGATATTTCTCCCGGCAATCTTGATTCTAGCC | A | G |
| 343 | GCCCAGCGTTTCTCATGATGTACTCTGCATATAAGTTAAATAAGCAGGGTGACAATATAnAGCCTTGACGTACTCCTTTTCCTATTTGGAACCAATCTGTTGTTCCATGTCCAGTTCTA | C | T |
| 344 | TCTCATGATGTACTCTGCATATAAGTTAAATAAGCAGGGTGACAATATATAGCCTTGACnTACTCCTTTTCCTATTTGGAACCAATCTGTTGTTCCATGTCCAGTTCTAACTGCTGCTT | A | G |
| 345 | TTAAATAAGCAGGGTGACAATATATAGCCTTGACGTACTCCTTTTCCTATTTGGAACCAnTCTGTTGTTCCATGTCCAGTTCTAACTGCTGCTTCCTGACCTGCATACAGGTTTCTCAA | A | G |
| 346 | TTTTTCTGGAACTCTCTTGCTTTTTCGATGATCCAGCAGATGTTGGCAATTTGATCTCTnGTTCCTCTGCCTTTTCTAAAACCAGGTTGAACATCTGGAAGTTCACAGTTCACGTATTG | G | T |
| 347 | TTCCAGAATTTAACACATCGTTTAACCAGACATATTCTTAGCAGCACTCAAGACGTTCCnTGGAGTGGGTGTATTTGCAAATTCTGGTCACGCAGAGCTGATTCACAACCACATTTCTT | A | G |
| 348 | CACGCAGAGCTGATTCACAACCACATTTCTTGCTCTGTGGCACCAGTCTTTTCCCCCAGnCTCTCTTCTGAGCTTCGTGTTATCCTTTGCTGAATTTCCCAAGGAGAAGGCGGAGAATC | T | C |
| 349 | TCTTGCTCTGTGGCACCAGTCTTTTCCCCCAGCCTCTCTTCTGAGCTTCGTGTTATCCTnTGCTGAATTTCCCAAGGAGAAGGCGGAGAATCTGGCTTCAGAATGTGTGTTTCCCTCAA | C | T |
| 350 | AGAATGTGTGTTTCCCTCAAAGAGACACCAAGCAGACCTTGCATGAGGACTGTCTCAGCnGAGATACCCGTCCTGCCCCTGGCCGTGTTTCTGGGGTTTGGGCCATAGACCCCGCAGGG | C | T |
| 351 | TCAAAGAGACACCAAGCAGACCTTGCATGAGGACTGTCTCAGCTGAGATACCCGTCCTGnCCCTGGCCGTGTTTCTGGGGTTTGGGCCATAGACCCCGCAGGGCAGACGGGGGGCTCTG | T | C |
| 352 | TGCATGAGGACTGTCTCAGCTGAGATACCCGTCCTGCCCCTGGCCGTGTTTCTGGGGTTnGGGCCATAGACCCCGCAGGGCAGACGGGGGGCTCTGGCCCTGGGCTCACCTTTGTTCCC | A | T |
| 353 | AAACCAAACTTGGATGCTCTCACTCACACGCAGTAAAGCCAGTCTCCTGACGCCAGGGTnGTGGTGAAGAAAAGGGCAGTGTTTCCTGGGTTGGGAATATTCCCTGGAGAAGGAAATGG | T | C |
| 354 | ATTCCCTGGAGAAGGAAATGGCAATCCACTCCAGCACTCTTGCCTGGAAAATCCCATGGnCGGAGGAACCTGGTAGGCTGCAGTCCACGGGGTCGCAAAGAGTCGGACACGACTGAGCG | A | G |
| 355 | GAAGGAAATGGCAATCCACTCCAGCACTCTTGCCTGGAAAATCCCATGGGCGGAGGAACnTGGTAGGCTGCAGTCCACGGGGTCGCAAAGAGTCGGACACGACTGAGCGACTTCAATTT | T | C |
| 356 | GGGCGGAGGAACCTGGTAGGCTGCAGTCCACGGGGTCGCAAAGAGTCGGACACGACTGAnCGACTTCAATTTCACTTTTACTTTACTGCAGGTGCCAAGCAAGGAGTCCAAGTGCTCAG | A | G |
| 357 | TGGGCTACAGGTTATGTGGGCTACAGGGTATCTGATCAGCTTGTGGACATTCTTCTTATnGGTTAACGTTGAGGTCATCGGGGGTCACCGTGGTCAGCCTTCTAGTTCCACCTGATCTG | T | C |
| 358 | TCCACCTGATCTGGGGTCCACCTGGTCTGTGGGCAGCATATAGTTACCTTCTTCCACCTnGTGTGGGGGGTGGTTCTCAGTATCTGCAGAAAAACTCACAGGACATGGCTCAGGATATT | G | GGT |
| 359 | GGTCCACCTGGTCTGTGGGCAGCATATAGTTACCTTCTTCCACCTGGTGTGGGGGGTGGnTCTCAGTATCTGCAGAAAAACTCACAGGACATGGCTCAGGATATTCTCCATAGCCCTGG | G | T |
| 360 | CCATAGCCCTGGTTTAGTCGCTAAGTCGTGTCTCACTCTTTTGTGACCCCATGGACTGTnGCCCACCAGGCTCCTCTGTCCATGGGATTCTCCAGGCAAGAATACTGGAGTGGGTTGCC | A | G |
| 361 | CTTTTGTGACCCCATGGACTGTGGCCCACCAGGCTCCTCTGTCCATGGGATTCTCCAGGnAAGAATACTGGAGTGGGTTGCCATAGCCCTTGAGGAGGAATTAAATGTCCTGGACTTTG | G | C |
| 362 | CACCAGGCTCCTCTGTCCATGGGATTCTCCAGGCAAGAATACTGGAGTGGGTTGCCATAnCCCTTGAGGAGGAATTAAATGTCCTGGACTTTGCTTAATGGCTAAGCTATTGCTGTATT | A | G |
| 363 | GGCTCCTCTGTCCATGGGATTCTCCAGGCAAGAATACTGGAGTGGGTTGCCATAGCCCTnGAGGAGGAATTAAATGTCCTGGACTTTGCTTAATGGCTAAGCTATTGCTGTATTTACTT | A | T |
| 364 | CTCTTTGGAAGTGCGTGAAAGCTTAGGAGGCTAAAGCTTTTTTACAGACAAGAGGCAGGnGTAGGACATGGGGTGGGGGAAAAGCCTGTTTTGGGGAGACCCCATGGGGTCCTGCTCAG | A | T |
| 365 | CTTTGGAAGTGCGTGAAAGCTTAGGAGGCTAAAGCTTTTTTACAGACAAGAGGCAGGTGnAGGACATGGGGTGGGGGAAAAGCCTGTTTTGGGGAGACCCCATGGGGTCCTGCTCAGTT | G | T |
| 366 | GGAAAAGCCTGTTTTGGGGAGACCCCATGGGGTCCTGCTCAGTTTCACTAGGACATTGGnGAGGGCAGTGACTCCTCCCTGCCCCAGGACATCGCTGACCATATTTTTCCTGATTTATT | C | A |
| 367 | ATCGCTGACCATATTTTTCCTGATTTATTTGAACTATAGTCCTGTAAACAAACCTTCATnAAAACTCAATCATGCATCCTCCTAACTCACGCACTCCTGCGTCTAGTCTGTCCACAAAC | C | T |
| 368 | AACAAACCTTCATTAAAACTCAATCATGCATCCTCCTAACTCACGCACTCCTGCGTCTAnTCTGTCCACAAACACGGACCCTGCTGGGAGATGAATGAGCTCATAGCCCTGACCTGGAG | T | G |
| 369 | CCTGGAGCAATTCTATCTCATCTCCCTTTTTTTATGTGTTTTCTCTCAGCAGCCCCACCnTGCTTTTAAGAAATTAGGAGACAAAGAAGGAGTGATGACTCTCTAAGCCGCTCAGTCTA | G | A |
| 370 | TCCCTACTAGTTAGATCCAGTCCAAACCATGTGTGTTGAGGCACGTGTGTTGCGGTGAAnTTCAGGAGGCCATTCTGACAATTAGCACAGTTGCCCAGGAAACAGCAGTGTTCTGGACG | C | G |
| 371 | GTATTGCAGTCCAGGTAACACGCCTGTCTCATCTGTGCCCTCTTAGCCACTTCTTCCGCnACACTCAGGACCAGGATCTCACCTGCTGCTTAGCTGCTCAGGCTTCAGCATGTGCAGCA | G | C |
| 372 | TATCTTAAATATCTTTACTAAGATAATCTTCTCAAAAATCTCCAAAGGCTCCTTATTGTnTAGTGAATTAAGTAAAATGTTTCTCTGACTTTTTGAGATTTCCAAATACGTAAACCTAG | T | C |
| 373 | AAAGTAGTCTGGTTGGATTCTTTTGCATGCAGCTGAAGAGTTTTCCTTACGAACAAAGCnAGTGGAGGTGATGGAACCCCACTTGAGCTACTTCAAATCCTGAAAGATGATGCTGTGAG | A | G |
| 374 | CAAAATTCTCCAAGCCAGGCTTCAGCAATATGTGATCCGTGAACTTCCAGATGTTCAAGnTGGTTTTAGAAAAGGCAGAGGAACCAGAGATCAAATTGCCAACATCTGCTGGATCATGG | C | A |
| 375 | AAAAGGCAGAGGAACCAGAGATCAAATTGCCAACATCTGCTGGATCATGGAAAAAGCAAnAGAGTTCCAGAAAAACATCTACTTCTGCTTTATTGACTATGCCAAAGCCTTTGACTGTG | C | G |
| 376 | AGAGTTCCAGAAAAACATCTACTTCTGCTTTATTGACTATGCCAAAGCCTTTGACTGTGnGGATCACAATAAACTCTGGAAAATTCTGAAAGAGATGGGAATACCAGACCTCCTGATCT | C | T |
| 377 | ATCTACTTCTGCTTTATTGACTATGCCAAAGCCTTTGACTGTGTGGATCACAATAAACTnTGGAAAATTCTGAAAGAGATGGGAATACCAGACCTCCTGATCTGCTTCTTGAGAAACCT | G | C |
| 378 | ATCACAATAAACTCTGGAAAATTCTGAAAGAGATGGGAATACCAGACCTCCTGATCTGCnTCTTGAGAAACCTGTATGCAGGTCATAAAGCAACAGTTAGAACTGGACGTGGAACAACA | C | T |
| 379 | TGCAGCCATGAAATTAAAAGACACTTACTCCTTGGAAGGAAAGTTATGACCAAACTAGAnAGCATATTGAAAAGCAGAGACATTACTTTGTCAACAAAGGTCTGTCTAGTCAAGGCTAT | C | T |
| 380 | TTATGACCAAACTAGATAGCATATTGAAAAGCAGAGACATTACTTTGTCAACAAAGGTCnGTCTAGTCAAGGCTATGGTTTTTCCAGTGGTCATGTATGGATGTGAGAGTAGGACTATA | C | T |
| 381 | GGAGTTGGTGATGGACAGGGAGGCCTGGCATGCTGCAGTTCATGGGGTCACAAAGAATCnGACATGACTGAATGACTGAACTGAAGAGTTTTCCCAGCACCATTTATTGAAGAGACTGT | G | A |
| 382 | GGTATATCAGTATAGTCAGTATAGATCAGTATAGATCAGTGTCAGTATAGTCACTTTAAnAATATTAACTCTTCCAATCTGTGAGCATGATATATTTTCCCCTCTATATCATCTTCAAT | T | C |
| 383 | GGTTAGAGTCATTCCTCGGTATTTTATTCCTTCTGGTACAATTGTGAATGAGATAATTTnCTTAGTTTCTCTTTCTGATAGCTCATTGTTAGTGTATAGAGAAGCAACAGATTTCTATG | C | T |
| 384 | TATCCTGCTATTTTACTGAATTCACTTATTAGCTTTTTGGTGACATCTAAAGATTTTCTnAAGAAAATGGCATGGTATGGTATGACAAGCTGTCAAGCCACCTGCAAACAGTGGTGGTT | C | T |
| 385 | CGGATTTCTTTTATTTCTTTTTGTCTGAGTACTGTGACTAGGATTCCCAGTACTGTATTnAATGAAAGTGGCAAGAGTGTACTTCCTTGCCTTATTTTTCTGGTCTTAGAGGAAATGCT | C | G |
| 386 | TTTCTTTTATTTCTTTTTGTCTGAGTACTGTGACTAGGATTCCCAGTACTGTATTGAATnAAAGTGGCAAGAGTGTACTTCCTTGCCTTATTTTTCTGGTCTTAGAGGAAATGCTCTCA | A | G |
| 387 | TTTTTGTCTGAGTACTGTGACTAGGATTCCCAGTACTGTATTGAATGAAAGTGGCAAGAnTGTACTTCCTTGCCTTATTTTTCTGGTCTTAGAGGAAATGCTCTCAGGTTTTCACCATC | A | G |
| 388 | TTGTCTGAGTACTGTGACTAGGATTCCCAGTACTGTATTGAATGAAAGTGGCAAGAGTGnACTTCCTTGCCTTATTTTTCTGGTCTTAGAGGAAATGCTCTCAGGTTTTCACCATCAAT | C | T |
| 389 | ATTATGATGTTTGCTATGGGCTTGTCATATGTGGCCTTTATTATATGGAGGTTTATTCCnTCTATACCCACTTTGTTGAGAGTTTGTATCATAAAAGTATGTTGAATTTTGTCAAATGG | T | C |
| 390 | TGCTATGGGCTTGTCATATGTGGCCTTTATTATATGGAGGTTTATTCCCTCTATACCCAnTTTGTTGAGAGTTTGTATCATAAAAGTATGTTGAATTTTGTCAAATGGTTTTCCTGCAT | T | C |
| 391 | CCCTCTATACCCACTTTGTTGAGAGTTTGTATCATAAAAGTATGTTGAATTTTGTCAAAnGGTTTTCCTGCATCTATTGAGATGATGTTTATTTTTCAATTCATTAATGATTTTTATTC | A | T |
| 392 | TGTATCATAAAAGTATGTTGAATTTTGTCAAATGGTTTTCCTGCATCTATTGAGATGATnTTTATTTTTCAATTCATTAATGATTTTTATTCTTCAATTTGTTAATGTGGTATATCCCA | T | G |
| 393 | CAATGATATTGACCTAAGGTTTTTTTTTTTTTTTTTGTAAAGTTTTTGTCTGGTTTTAGnATCAGGGTGATGCTGGCCTCATAGAGAGAGTTTAGAAGCATTTCCTTCTCTTTGATTTT | T | A |
| 394 | GTCTGGTTTTAGAATCAGGGTGATGCTGGCCTCATAGAGAGAGTTTAGAAGCATTTCCTnCTCTTTGATTTTTTGGAATAGTTTGAGTAGGATAGGTATTAACTCTTCTTTAAAGGTCT | C | T |
| 395 | CACCCACTGTGTGCCCACGGGCCACAGCCAGAGAAAACCCACAGACGGCAATGAAGTCCnAGCACAACCAGAAAAAGAAGTTCGGTAGATACAGCTGTGAAGCCCTCTGGTCCTGGACT | T | C |
| 396 | CATATTTTCTATTTCTTCCTGGCTCAGTCTTGGGTTTGTACATGTCTAGGAATGTATCCnTTTCTTCTGGGTTGTCCCTTTTATTAGACATGTGTGGGGGCACACAGCACCGACCAGTG | A | G |
| 397 | CATGTGTGGGGGCACACAGCACCGACCAGTGAGACTCATACCGGCTTCCTGGGGCCAGGnTGCGGGCCCCAAGCAGCACGGCATCCTAGACTGTGTGAATGCCCACTGACCCTGCCCAG | G | A |
| 398 | GTGTGGGGGCACACAGCACCGACCAGTGAGACTCATACCGGCTTCCTGGGGCCAGGATGnGGGCCCCAAGCAGCACGGCATCCTAGACTGTGTGAATGCCCACTGACCCTGCCCAGCCC | G | C |
| 399 | CCAGCCCCACAGTTTCATTCTGAGAGAAGTGATTTCTTGCTTCTGCACTTACAGGCCCAnGACCTGACCTGCTTCTGAAGAGCAGGGGTTTTGGCAGGAGGGGGAGATGCTGAGAGCCG | G | A |
| 400 | ACCTGCTTCTGAAGAGCAGGGGTTTTGGCAGGAGGGGGAGATGCTGAGAGCCGATGGGGnTCCAGGTCTCCTCCCAGGTCCCACTCTCTGGGGCAGCGCTTGGAAAAGATTGTCCAGCC | A | G |
| 401 | AGCTGAAGGTTCCTGGAAGTTATGAATAGCTTTGCCATGAAGGGCATGGTTTGTGGTCAnGGTTCACAGGAACTTGGGAGACCCTGCAGCTCGGACGTCCTGAGGTTGGTGGCACCCTG | C | T |
| 402 | TGCCATGAAGGGCATGGTTTGTGGTCATGGTTCACAGGAACTTGGGAGACCCTGCAGCTnGGACGTCCTGAGGTTGGTGGCACCCTGATTTCCTAAGCTCGCTGGGGAACGGGGTGCTA | T | C |
| 403 | GGGCATGGTTTGTGGTCATGGTTCACAGGAACTTGGGAGACCCTGCAGCTCGGACGTCCnGAGGTTGGTGGCACCCTGATTTCCTAAGCTCGCTGGGGAACGGGGTGCTACTTCTCCCT | C | T |
| 404 | GGACGTCCTGAGGTTGGTGGCACCCTGATTTCCTAAGCTCGCTGGGGAACGGGGTGCTAnTTCTCCCTGGCTGACCTCCCTCTGCTCTGCATCACCCAGTTCTGAGAGCAGAGTGGTGC | T | C |
| 405 | TGCTACTTCTCCCTGGCTGACCTCCCTCTGCTCTGCATCACCCAGTTCTGAGAGCAGAGnGGTGCTGGGGGCACAGCCTCTCGCATCTGACACTTGTGTTCAAACCACCCATGCTGGTG | C | T |
| 406 | TTCTCCCTGGCTGACCTCCCTCTGCTCTGCATCACCCAGTTCTGAGAGCAGAGTGGTGCnGGGGCACAGCCTCTCGCATCTGACACTTGTGTTCAAACCACCCATGCTGGTGTTCGGGG | TG | T |
| 407 | AGTGGTGCTGGGGGCACAGCCTCTCGCATCTGACACTTGTGTTCAAACCACCCATGCTGnTGTTCGGGGGGCCACCTATGGGGAAGGCTCCTCACTGCAGGGGTGCCCCTGTCCCCTGA | C | G |
| 408 | GGTGTTCGGGGGGCCACCTATGGGGAAGGCTCCTCACTGCAGGGGTGCCCCTGTCCCCTnAGAGATCAGAAGTCCCAGTCTGGATGTCGAATGGCCGAGCTCCCTCCAGAGGCTCCAGG | C | G |
| 409 | GGGGAAGGCTCCTCACTGCAGGGGTGCCCCTGTCCCCTGAGAGATCAGAAGTCCCAGTCnGGATGTCGAATGGCCGAGCTCCCTCCAGAGGCTCCAGGGAGGGATCCTTGCCCCCTCCG | C | T |
| 410 | AAGGCTCCTCACTGCAGGGGTGCCCCTGTCCCCTGAGAGATCAGAAGTCCCAGTCTGGAnGTCGAATGGCCGAGCTCCCTCCAGAGGCTCCAGGGAGGGATCCTTGCCCCCTCCGCCGC | C | T |
| 411 | TCCAGAGGCTCCAGGGAGGGATCCTTGCCCCCTCCGCCGCCGCCTCCAGCTCCTGGTGCnGCACCCTTGGGCCCGATCTCGTAGACGCCTCAGTCCAGTCTCTGCCTCCGTGTTCACTG | T | C |
| 412 | AGCTCCTGGTGCCGCACCCTTGGGCCCGATCTCGTAGACGCCTCAGTCCAGTCTCTGCCnCCGTGTTCACTGGCATTCTCCCCATGTCCCCTCTGTGTCCCCGTTTTCTCTCACAAGGA | C | T |
| 413 | CCGCACCCTTGGGCCCGATCTCGTAGACGCCTCAGTCCAGTCTCTGCCTCCGTGTTCACnGGCATTCTCCCCATGTCCCCTCTGTGTCCCCGTTTTCTCTCACAAGGACACCGGACATA | G | T |
| 414 | GACCTAGATTCCAAACAAGATTCCATCCTGAAGTTCCTGGTGGACGTGAGTTCTGGAGCnACGCCCTTCAACCCCATCACAGCTTGCGGTTCATCGCAAAACACGGAACCTGGGATTTA | A | G |
| 415 | AGCTTGCGGTTCATCGCAAAACACGGAACCTGGGATTTATCGTAAAACCCAGGTTCTTCnTGAAACACTGAGCTTCGAGGCTTGTTGCAAGAATTAAAGGTGCTAATACAGATCAGGGC | A | G |
| 416 | AACAGTCTCTCCGGGAAGGAAACCAGAGGCCAGAGAGCAAGCCAGAGCTAGTCTAGGAGnTCCCTGAGCCTCCACCCAAGATGCCGACCAGGCCAGCGGGCCCCCTGGAAAGACCCTAC | G | A |
| 417 | TCCCTGAGCCTCCACCCAAGATGCCGACCAGGCCAGCGGGCCCCCTGGAAAGACCCTACnGTCTAGGGGGGGAACAGGAGCCGACCCGCCAGGCCCCCGCTATCAGGAGACACCCCAAC | C | A |
| 418 | AGGGGGGGAACAGGAGCCGACCCGCCAGGCCCCCGCTATCAGGAGACACCCCAACCTTGnTCCTGTTCCCCTACCCCAGTACGCCCACCCGACCCCTGAGATGAGTGGTTTACTTGCTT | G | C |
| 419 | CAGGCCCCCGCTATCAGGAGACACCCCAACCTTGCTCCTGTTCCCCTACCCCAGTACGCnCACCCGACCCCTGAGATGAGTGGTTTACTTGCTTAGAATGTCAATTGAAGGCTTTTGTA | A | C |
| 420 | CACCCCAACCTTGCTCCTGTTCCCCTACCCCAGTACGCCCACCCGACCCCTGAGATGAGnGGTTTACTTGCTTAGAATGTCAATTGAAGGCTTTTGTACCCCCTTTGCCAGTGGCACAG | C | T |
| 421 | ACCCCAACCTTGCTCCTGTTCCCCTACCCCAGTACGCCCACCCGACCCCTGAGATGAGTnGTTTACTTGCTTAGAATGTCAATTGAAGGCTTTTGTACCCCCTTTGCCAGTGGCACAGG | A | G |
| 422 | ACCTTGCTCCTGTTCCCCTACCCCAGTACGCCCACCCGACCCCTGAGATGAGTGGTTTAnTTGCTTAGAATGTCAATTGAAGGCTTTTGTACCCCCTTTGCCAGTGGCACAGGGCACCC | G | C |
| 423 | CCCTCCCTGCTCGGGCCCCCTCCATACTCAGCGACACACCCAGCACCAGCATTCCCACCnCTCCTGAGGTCTGAAGGCAGCTCGCTGTGGTCTGAGCGGTGCGGAGGGAAGTGCCCTGG | G | A |
| 424 | GAGGGAAGTGCCCTGGGAGATTTAAAATGTGAGAGGTGGGAGGTGGGAGGTTGGGTCCTnTAGGCCTTCCCATCCCACGTGCCTGCACGGAGCCCTAGTGCTACTCAGTCATGCCCCCG | C | G |
| 425 | GTGAGAGGTGGGAGGTGGGAGGTTGGGTCCTGTAGGCCTTCCCATCCCACGTGCCTGCAnGGAGCCCTAGTGCTACTCAGTCATGCCCCCGCAGCAGGGGTCAGGTCACTTTCCCATCC | T | C |
| 426 | AGGCCTTCCCATCCCACGTGCCTGCACGGAGCCCTAGTGCTACTCAGTCATGCCCCCGCnGCAGGGGTCAGGTCACTTTCCCATCCTGGGGGTTATTATGACTGTTGTCATTGTTGTTG | C | A |
| 427 | GGGCAGCGGGTGCTTGCAGAGCCCTCGATACTGACCAGGTTCCCCCCTCGGAGCTCGACnTGAACCCCATGTCACCCTCGCCCCAGCCTGCAGAGGGTGGGGTGACTGCAGAGATCCCT | G | C |
| 428 | ATACTGACCAGGTTCCCCCCTCGGAGCTCGACCTGAACCCCATGTCACCCTCGCCCCAGnCTGCAGAGGGTGGGGTGACTGCAGAGATCCCTTTACCCAAGGCCACAGTCACATGGTTT | G | C |
| 429 | AAGTGTTCCTGGCGCTGGCAGCCAGCCTGGACCCAGAGCCTGGACACCCCCTGCGCCCCnACTTCTGGGGCGTACCAGGAACCGTCCAGGCCCAGAGGGGGCCTTCCTGCTTGGCCTCG | T | C |
| 430 | CGCTGGCAGCCAGCCTGGACCCAGAGCCTGGACACCCCCTGCGCCCCCACTTCTGGGGCnTACCAGGAACCGTCCAGGCCCAGAGGGGGCCTTCCTGCTTGGCCTCGAATGGAAGAAGG | C | G |
| 431 | GAATGGAAGAAGGCCTCCTATTGTCCTCGTAGAGGAAGCAACCCCAGGGCCCAAGGATAnGCCAGGGGGGATTCGGGGAACCGCGTGGCTGGGGGCCCGGCCCGGGCTGGCTGGCTGGC | C | G |
| 432 | TATAAGGCCCCGAGCCCACTGTCTCAGCCCTCCACTCCCTGCAGAGCTCAGAAGCGTGAnCCCAGCTGCAGCCATGAAGTGCCTCCTGCTTGCCCTGGCCCTCACTTGTGGCGCCCAGG | T | C |
| 433 | AGGCCCCGAGCCCACTGTCTCAGCCCTCCACTCCCTGCAGAGCTCAGAAGCGTGACCCCnGCTGCAGCCATGAAGTGCCTCCTGCTTGCCCTGGCCCTCACTTGTGGCGCCCAGGCCCT | G | A |
| 434 | CTCAGAAGCGTGACCCCAGCTGCAGCCATGAAGTGCCTCCTGCTTGCCCTGGCCCTCACnTGTGGCGCCCAGGCCCTCATTGTCACCCAGACCATGAAGGGCCTGGATATCCAGAAGGT | C | T |
| 435 | GCAGCCATGAAGTGCCTCCTGCTTGCCCTGGCCCTCACTTGTGGCGCCCAGGCCCTCATnGTCACCCAGACCATGAAGGGCCTGGATATCCAGAAGGTTCGAGGGTGCCCGGGTGGGTG | C | T |
| 436 | CAGGCCCTCATTGTCACCCAGACCATGAAGGGCCTGGATATCCAGAAGGTTCGAGGGTGnCCGGGTGGGTGGTGAGTTGCAGGGCAGGCAGGGGAGCTGGGCCTCAGAGACCAAGGGAG | G | C |
| 437 | GGGCAGCTTCAACCAGGCGTTTAGTGTCTTGCATTCTGGAGGCTGGAAGCCTGCAATCCnGGCATCGGCCCAGCTGGCTTCTCCTGCGGCCACTCTCCGGGGAGCAGACAGCCATCTTC | A | G |
| 438 | CCTCTTCCTGTGAGGTCACCAGGCCTGCTGGATCCACGCCCGCCCACACAGCCTCACGTnACCTTTGTCATCTCTTTAAAGGCCGTGTCTCCAGTCCTGTGTTGAGGTTCTGGGGGTTA | G | A |
| 439 | AGAGTCACTCTGCCCCTCAAATTTTCCCCACCTCCAGCTATGTCTCCCCAAGATCCAAAnGTTGCCACGTGTGCGGGGGCTCATCTGGGTCCCTCTTTGGGCTCAGAGTGAGTCTGGGG | C | T |
| 440 | CAGGGCTGCCCAGGCCAGGGTGGGACAGAGAGCCCACTGTGGGGCTGGGGGCCCCTTCCnGCCCCTGGAGTGCAGCTCAAGGTCCCTCCCCAGGTGGCGGGGACTTGGTACTCCTTGGC | G | C |
| 441 | GAAGCCCACCCCTGAGGGCGACCTGGAGATCCTGCTGCAGAAATGGTGGGCGTCCCCCCnAAAAAAAGCATGGAACCCCCACTCCCCAGGGATATGGACCCCCCCGGGGTGGGGTGCAG | C | CA |
| 442 | GGGCTTGGAGTTTCCTGGTACCCCTGGAGGTCCACCCAAGGCTGCTTATCCAGGGCTTTnTCTTTCTTTTTTTCCCCCAACTTTTATTAATTTGATGCTTCAGAACATCATCAAACAAA | T | C |
| 443 | AGATGGGGACCTGAACCCCAGGACTGCCTTTTGGGGTGCCTGTGGTCAAGGCTCTCCCCnACCTTTTCTCCCTGGCTCCATCTGACTTCTCCTGGCCCATCCACCCGGTCACCTGTGGC | G | A |
| 444 | CTTCTCCTGGCCCATCCACCCGGTCACCTGTGGCCCCAGAGGTGACAGTGAGTGCAGCCnAGGCCGGTTGGCCAGCCGGCCCCCTATGCCCACGCCACCCGCCTCCAGCCCCTCCTGGG | G | A |
| 445 | CACCCGGTCACCTGTGGCCCCAGAGGTGACAGTGAGTGCAGCCAAGGCCGGTTGGCCAGnCGGCCCCCTATGCCCACGCCACCCGCCTCCAGCCCCTCCTGGGGCCGCCTTCTGCCCCT | T | C |
| 446 | TCCTGATGAAAATGGTCCATGCCCGTGGCTCAGAAAGCAGCTGTCTTTCAGGGAGAACGnTGAGTGTGCTCAGAAGAAGATCATTGCAGAAAAAACCAAGATCCCTGCGGTGTTCAAGA | A | G |
| 447 | GGGACGCCCACCACCCCCCAGGGACTGTGGGCAGGTGCAGGGGGCTGGCGTCAGGCCCCnAGATGCTAAGGGGCTGGTGGTGATGAAGACACTGCCGTGCCACCTGCTTCCCTGGCCTC | A | G |
| 448 | GGACGCCCACCACCCCCCAGGGACTGTGGGCAGGTGCAGGGGGCTGGCGTCAGGCCCCGnGATGCTAAGGGGCTGGTGGTGATGAAGACACTGCCGTGCCACCTGCTTCCCTGGCCTCC | G | A |
| 449 | GATGAAGACACTGCCGTGCCACCTGCTTCCCTGGCCTCCCTGCCACCTGCCCGGGGCCTnGGGGCCGGTGGCCGTGGGCAGGTCCCGGCTGGGCAGGTCTGACACCCCAGGGTGACACC | C | T |
| 450 | ATGAAGACACTGCCGTGCCACCTGCTTCCCTGGCCTCCCTGCCACCTGCCCGGGGCCTTnGGGCCGGTGGCCGTGGGCAGGTCCCGGCTGGGCAGGTCTGACACCCCAGGGTGACACCC | A | G |
| 451 | GCAGGTCTGACACCCCAGGGTGACACCCGAGCTCTCTTTGCTGAGGGTGGGGTGGTGCTnGGGGCCCTCAGGCTGAGCTCAGGAGGTCCCTGTGCCCACCCAGGGGTAACCGAGAGCCG | T | C |
| 452 | TGGGGTGGTGCTCGGGGCCCTCAGGCTGAGCTCAGGAGGTCCCTGTGCCCACCCAGGGGnAACCGAGAGCCGCTGCCCGCTCCAGGGGTCCAGGTGCCCCACGATCCCAGCCCACCCCA | T | TA |
| 453 | CACATCTGCCCTTGGCCTCTTCAGGACTCACTCTGACTGGAGGCCCTGCACTGACTGATnCCAGGGTGCCCAGCCCAGGGTCTCCTGTGCCATCCGGCTGCACGGGGTTTGGATGCTGG | A | G |
| 454 | GGGGTTTGGATGCTGGTCCTGCCCCCAAGCTGCCCAGACACTGCAGGGCAGCTGGGGCCnCCCGCAGGCCTCGGTCAGGGAGAGCCCCAGCTGCCCCCGCTCAGCGCTGCCCCCCAACA | G | A |
| 455 | CACTGCAGGGCAGCTGGGGCCACCCGCAGGCCTCGGTCAGGGAGAGCCCCAGCTGCCCCnGCTCAGCGCTGCCCCCCAACAATTCCCCAGTCCTCAGGACGCATCCCTCTTCCCTTGCT | T | C |
| 456 | GTGGAGGGTCCCTGGCGGATCCAGAGTTGGGCTTCCAGAGTGAGGGCTTCCTGGGCCCCnTGTGCCTGGCAGTGGCAGCAGGGAAGGGGCCACACCATTTTGGGGCTGGGGGATGCCAG | G | A |
| 457 | TCCCTGGCGGATCCAGAGTTGGGCTTCCAGAGTGAGGGCTTCCTGGGCCCCATGTGCCTnGCAGTGGCAGCAGGGAAGGGGCCACACCATTTTGGGGCTGGGGGATGCCAGAGGGCGCT | A | G |
| 458 | GAGGGCTTCCTGGGCCCCATGTGCCTGGCAGTGGCAGCAGGGAAGGGGCCACACCATTTnGGGGCTGGGGGATGCCAGAGGGCGCTCCCCACCCCGTCCTCACCAAGTGGTGACCCCGG | C | T |
| 459 | AGGGCTTCCTGGGCCCCATGTGCCTGGCAGTGGCAGCAGGGAAGGGGCCACACCATTTTnGGGCTGGGGGATGCCAGAGGGCGCTCCCCACCCCGTCCTCACCAAGTGGTGACCCCGGG | A | G |
| 460 | CTCCCCACCCCGTCCTCACCAAGTGGTGACCCCGGGGGAGCCCCGCTGGTTGTGGGGGGnGCTGGGGGCTGACCAGAAACCCCCCTCCTGCTGGAACTCACTTTCCTCCTGTCTTGATC | C | T |
| 461 | GGGGGCTGACCAGAAACCCCCCTCCTGCTGGAACTCACTTTCCTCCTGTCTTGATCTCTnCCAGCCTTGAACGAGAACAAAGTCCTTGTGCTGGACACCGACTACAAAAAGTACCTGCT | A | T |
| 462 | GAAACCCCCCTCCTGCTGGAACTCACTTTCCTCCTGTCTTGATCTCTACCAGCCTTGAAnGAGAACAAAGTCCTTGTGCTGGACACCGACTACAAAAAGTACCTGCTCTTCTGCATGGA | C | T |
| 463 | CTACAAAAAGTACCTGCTCTTCTGCATGGAGAACAGTGCTGAGCCCGAGCAAAGCCTGGnCTGCCAGTGCCTGGGTGGGTGCCAACCCTGGCTGCCCAGGGAGACCAGCTGTGTGGTCC | T | C |
| 464 | GGGTCCCCGAGTCCCGCCAGGAGAGGGTGGTCATATACCGGGAGCCGGTGTCCTGGGGGnCTGTGGGTGACTGGGGACGGGGGCCAGACACACAGGCTGGGAGACGGGGGGCTGCAGCG | T | C |
| 465 | TGCCACTGGGCATTTTCAGGGCCCATGTGCCAGGAGGGCGTGGGCATCGGCGAGTGGAGnCTCCTGGCCGTGTCAGCTGGCCCAGGGGGAGGAGGGGACCCAGACAGCCAGAGGTGGGG | T | G |
| 466 | CCCCCTCCCCACCCCCTCCCCAGCTCCCTCTGTCCTGGGGTGTCCAGTCCCATCCTGACnCTCCCCCGCCACGGCTCTCCCTCCCCCACAGAGCAGTGCCACATCTAGGTGAGCCCCTG | A | G |
| 467 | CCCTCCCCACCCCCTCCCCAGCTCCCTCTGTCCTGGGGTGTCCAGTCCCATCCTGACGCnCCCCCGCCACGGCTCTCCCTCCCCCACAGAGCAGTGCCACATCTAGGTGAGCCCCTGCC | C | T |
| 468 | CCAAGGAGGGGCCGTCCTGAATCCCCAGCCACGGACAGGCTGGCAAGGGTCTGGCAGGTnCCCCAGGAATCACAGGGGAGCCCCATGTCCATTTCAGAGCCCGGGAGCCTTGGCCCCTC | G | A |
| 469 | GCATCACCGACAAAATGGACATGGGTTTGGGTGGACTCCAGGAGTTGGTGATGGACAGAnAGGCCTGGCATGCTGCGGTCCATGGGGTCACAAAGAGTCGGACACGACTGAGCGACTGA | G | A |
| 470 | ACTCATATTTATATATATACGTGAATGCTCAGTCACACTCAGTCATATCTGACTCTGTGnCCCATGGACTGTAGCCCTCCAGGCTCCTTCTGTCCACAGAATTCTCCAGGCAAGAATAC | G | A |
| 471 | ATCCCCTAGCCTGGAAGGCAGATTCTTAACCACTGGACCACCAGGGAAGTCCCCTGCCTnCCTGCATCTTAACCTCAGTTCAGTCGCTCAGTTGTGTCCGACTCTTTGCGACCCGATGA | A | G |
| 472 | GACACAGCATTGCTGTCCGGCGAATGGAGCCACCATCTGGATCCTTCAAGTGACCCTGGnCGGGGCAGGGCTGGCACTGGCCGTCCGGCCTGCACTTCCGTCGGGCTCCGGACTCCAGG | G | A |
| 473 | ACAAGAAGTGTGGGAGAAATGACCAAGATATCTTGAGGGAAAAAAATAAGGTTATATATnTAACTCTGCACATTATCTTAAAATTAATTCTGAATGAAAAGAAATCTTGAAAGCTCTAT | C | A |
| 474 | TATACCTTAAAAACTAAAAAAATAAAATAAAATAGGCTTTGATGTCCCAAGCAATCAAAnAAAAAGATTCTATGTTTGCTTATCAAATTAGCAAAATTTTTTAAAAATTTAACTCAGCA | G | A |
| 475 | TTTTCGGAAGGGCATCACCAAACACTATCCACATGGGAGGCGCCAGGTCGACACTGCAGnATTCCCAGGGAGGAAGGCAGGAGGTCGGGCCTGTATTTCAGCTGCAATGGTTACAGAAG | G | A |
| 476 | TGTTTTGTAGAATAATATTAGAAACCAAGGTCTGGGGCTGGGAGTGCTCGCTGCTCCTGnGGGCTCCTCGTTTCTAGGTCCTCTCAGCTGGGCAAGCAGGGAAGGCATGTGTGTAGTTG | C | T |
| 477 | CTGATGCTGGAAAAGATTGATGGCAGGATGATAAGGGGGCAACCGAGGATAAGATGCTTnGATGGCATCACCAACTCAATGGACATGAATTTAAGCAGATTCCAGGAGTTGGTGAAGGA | T | G |
| 478 | AAGGGGGCAACCGAGGATAAGATGCTTGGATGGCATCACCAACTCAATGGACATGAATTnAAGCAGATTCCAGGAGTTGGTGAAGGACAGGGAAGCCTGGCGTGCTGCAGTCCATGGGC | T | TA |
| 479 | CCATGGGCTGAGAAACAACAAAGCACAGACTCCACTGGTTTCCAAGGTGACTTCTCTCAnCACCTTCCTCAGCCCTCAGGGAGCTTACTTCTGGCATGTTTGATACAGTTCGATGACTT | G | A |
| 480 | CTTTACTGTCTCTGCAGTTTTGCCTTTTCCAGAATTTCACGTGGCTGGAATCATACAGTnGGTAGTCTTTTCAGATTGGCTTCTGGCTTCTTTCCCTTACCAATGTGTACTGATACTTC | G | A |
| 481 | GTGTCCGACTCTGTGTGACCCCATAGACTATACAGTCCATGGAATTCTCCAGGCCAGAAnACTGGAGTGGATAGCCATTCCCTTCTCCAGGGGATCTTCCCAACCCAGGGATCGAACTC | T | C |
| 482 | TAGGCTATCCCAGGGACTTCCCTGGTGGCCCAGTGGCTAGGACTCTGCGGTTCCAGTTCnGGGGGCCTGGGTTTGACCCCAGGTCAGGGAACTAGATCCCACTTGCCAAAACTAAGGGT | A | AG |
| 483 | CAAAAATATACTTTCCAGTTTCATTGCTGATAGAACTGATCTCGGAAACAGAGGGTCTGnATCAAGGTCACCTAGTAGGTTACAGGACCAGATGTAGTTTCCATCTCTGTCTGCACCAT | A | C |
| 484 | ATACTTTCCAGTTTCATTGCTGATAGAACTGATCTCGGAAACAGAGGGTCTGAATCAAGnTCACCTAGTAGGTTACAGGACCAGATGTAGTTTCCATCTCTGTCTGCACCATTATCATT | G | A |
| 485 | TTTCATTGCTGATAGAACTGATCTCGGAAACAGAGGGTCTGAATCAAGGTCACCTAGTAnGTTACAGGACCAGATGTAGTTTCCATCTCTGTCTGCACCATTATCATTAGGTGATTCTG | G | A |
| 486 | TTGCTGATAGAACTGATCTCGGAAACAGAGGGTCTGAATCAAGGTCACCTAGTAGGTTAnAGGACCAGATGTAGTTTCCATCTCTGTCTGCACCATTATCATTAGGTGATTCTGTGTTG | C | T |
| 487 | CTGATAGAACTGATCTCGGAAACAGAGGGTCTGAATCAAGGTCACCTAGTAGGTTACAGnACCAGATGTAGTTTCCATCTCTGTCTGCACCATTATCATTAGGTGATTCTGTGTTGCAG | G | A |
| 488 | ATCTCGGAAACAGAGGGTCTGAATCAAGGTCACCTAGTAGGTTACAGGACCAGATGTAGnTTCCATCTCTGTCTGCACCATTATCATTAGGTGATTCTGTGTTGCAGAGCCAAGTAGCA | T | C |
| 489 | AGGGTCTGAATCAAGGTCACCTAGTAGGTTACAGGACCAGATGTAGTTTCCATCTCTGTnTGCACCATTATCATTAGGTGATTCTGTGTTGCAGAGCCAAGTAGCACTACTATGATTAC | C | T |
| 490 | GTCTGAATCAAGGTCACCTAGTAGGTTACAGGACCAGATGTAGTTTCCATCTCTGTCTGnACCATTATCATTAGGTGATTCTGTGTTGCAGAGCCAAGTAGCACTACTATGATTACATA | C | A |
| 491 | CTGAATCAAGGTCACCTAGTAGGTTACAGGACCAGATGTAGTTTCCATCTCTGTCTGCAnCATTATCATTAGGTGATTCTGTGTTGCAGAGCCAAGTAGCACTACTATGATTACATATC | C | T |
| 492 | AATCAAGGTCACCTAGTAGGTTACAGGACCAGATGTAGTTTCCATCTCTGTCTGCACCAnTATCATTAGGTGATTCTGTGTTGCAGAGCCAAGTAGCACTACTATGATTACATATCTTT | T | A |
| 493 | AAGGTCACCTAGTAGGTTACAGGACCAGATGTAGTTTCCATCTCTGTCTGCACCATTATnATTAGGTGATTCTGTGTTGCAGAGCCAAGTAGCACTACTATGATTACATATCTTTTCCT | C | A |
| 494 | GTAGGTTACAGGACCAGATGTAGTTTCCATCTCTGTCTGCACCATTATCATTAGGTGATnCTGTGTTGCAGAGCCAAGTAGCACTACTATGATTACATATCTTTTCCTGTAGCCTTTTG | T | A |
| 495 | GTACCATCTCCCTCTGGGACGGTCGGTGCTGCCACCATCCCCCAGGAGTGACAACTGAGnCCAGTCAAGGACACTCTCAGATACGCCTCCTCACAGTCAGGATACCCTATGCTACTCGC | C | T |
| 496 | CCTGAGATAGTTACTTAAATTCTCTATACCACATGTTTCCTCATTCTGAAAACAGAGATnATGATAGTACTTTTACAAATGCTATTGCTGTAGAGATTAAATGTATTAATCCAGGTAAA | A | C |
| 497 | CAGGCACAATAGCAAGGCCCCTCTTGTGTTATTTTATGTTATTTTCCCCTCAACCTCTTnTTGTACTGTTTTTTCTGGATATTATTTTATGTTATTTTCCCCTAAACCTCTTATTATTC | A | G |
| 498 | TTTCAGCCATTGGCCACTACACGTCTCCTTTTTCTACTAATATCTGATCTAAAGCAATTnCATTTTCTGAAAAATTCAATAGTTATAAGAGGATACCTTGAAACCATAATTACAGCTAC | T | C |
| 499 | CAATTAAAATAAATAAATTAAAAATATATATAAAATATAAATAAAAATTTTTCAGGATTnAAAAAAAAGGTCACAACACTGGAATTTCAAGTTACCAAAATCTGCTGCTAAGCTAAGTC | A | T |
| 500 | CTCCTGCCTGTGCCTCCATGTGGGGCCCCTTTCATTTATTTTCCACCTATCCTCAGAGCnTTGTTCAAATTCCGCCTCCTCCAGGAAGCCTTCCGGGTCAATCCAGCTGGAAATGCGCT | C | T |
| 501 | CCCAAGCTTATTCTTCTATCCATGAGACTCTCCTGCAAGCACCCTCCTCTGTAGGCTTCnGCTTCCCCACCTGCAGTAATCAGAGGGGCCATCAGTGAGGGCCTCCTGAGGGCCAGGCA | G | A |
| 502 | ACAATTATAAACAATGCATGGTGCTTGTGGAAACCCAGACTCAAGGCATTTATGAAAACnAAACTCCCCCGTGGAGGGCAGGAAGGGCAGTGGGATAGAGGAGTAGAGGAATGCAATGG | C | G |
| 503 | CCTGCCACGCAGAAGAGGCAGGTTCAATCCCTGGGTCAGGAAGAGAAGGAAATGGCAGCnCACTCCAGTATCCCATGACAGAGGAACCTGGTAGGTATAGTCTATGGGGTTGCAAAAGA | G | A |
| 504 | ATAGGTATGATGCAGTTCATAATAAAATGTCTAAAAAGAGACCAAAATAAATAAAGAAGnGATGAGAGGACTTTCTGGTAGTCCAACGATTAAGAATCCACCCACCAGTGCAGGGAATG | T | C |
| 505 | AATGAGTAGTTTCTAGTGACATGAAAAAACGCTGATAGTGACATGAAAAAATACAGATAnAAAAAAAAAGATAATAGTGAAAAAAAGCTGGATGCAGAACACATTAACAGCATGGTCTG | G | GA |
| 506 | AAACTCCCAGCCATACCAGGTATTTCTCTGGTGTATAGATAGGCTTTCTAAAGCACCATnAGCATTTCAGACTCCTGTTTACCCAACAAGGATCATATACCTTTAAATTGCTTCCCCTC | G | A |
| 507 | TCCCATTCTGTTGTTTTCCTCTATTTCTTTGCATTGCTGAGGAAGGCTTTCTTATCTCTnCTTGCTATTCTTTGGAACTCTGCATTCAAATGAGTATATCTTTCCTTTACTCCTTTGCT | C | T |
| 508 | TTTCATGCACTGGAGGAGGAAATGGCAACCCACTCCAGTGTTCTTGCCTGGAGAATCCCnTGGACAGAGGAGCCTGGTGGGCTGCCGTCTATGGGGTCGCACAGAGTCGGACACGACTG | G | A |
| 509 | TTCATGCACTGGAGGAGGAAATGGCAACCCACTCCAGTGTTCTTGCCTGGAGAATCCCGnGGACAGAGGAGCCTGGTGGGCTGCCGTCTATGGGGTCGCACAGAGTCGGACACGACTGA | T | G |
| 510 | ACATTAGTTTTAGGCTTCTTTGTAATTTTTTCTTGTTTGTTGATGGTTTGACAAAAGGCnAAGTTTTTAAATACTCGGTTTTGAAAGGTGTTTTTTTATTTTTTATTTAAGTGTATTTG | G | A |
| 511 | TCTTTTTGTTTGGAGCAACCTCATGAAGTTGTTTTGTTTTTTGTTTTTTAACAGTCAAAnTGTATGGCTCATTGTAGAAAATACAGAAACATATAAAGAATTTACTAGTTCATCATCCC | C | T |
| 512 | ATCACACATATATTCCTGATAGGAATGTAAGATAGGACAATCACTCTGGAAAATACTTCnTCAGTCTCCTCTAAAACTAAATATATATGTACCGTGTGTATGTGCTCAGACCCTGAGTC | G | A |
| 513 | GTCACACAGGGATTGGTGAACTACAGACCCTCAGATATGGCAGGGAGCCACGAGCTCGTnGTCTTTGACTCCCATCCCCAACTCTATACCTGAATCCTGCCCCATCCCTCAGCCCTCTC | C | T |
| 514 | TGTCTCTGCTTTTGAATATGCTATCTAGCTTGGTCATAACCTTCCTTCCAAGGAGTAAGnGTATTTTCATTTCATGGCTGCAGTCACCATCTGCAGTGATTTTGGAGCCCAAAAAAATA | C | T |
| 515 | GAGTAAGCGTATTTTCATTTCATGGCTGCAGTCACCATCTGCAGTGATTTTGGAGCCCAnAAAAATAAAGTCTGACACTGTTTCCACTATTTCCCCATCTATTTCCCATGAAGTGATGG | A | G |
| 516 | GGTTCACATATTGCTGAAGCCTGGCTTGGAGAATTTTGAGCATTACTTTACTAGCGTGTnAGATGAGTGCAATTGTGCAGTAGTTTGAGCATTCTTTGGCATTGCCTTTCTTTGGGATT | GAGATG | GAGATG |
| | | AGTGCA | AGTGCA |
| | | ATTGTG | ATTGTG |
| | | CA | CG |
| 517 | GGCCAGGGGTCCCCAAATGGAGGAGACAGACTACAAGTGTCAGACATTTTAAACTATCTnTTAAGTGGCAGGAGGAAACAAACAAGTGTTAGATTTCTTCCCCTCCTCTATACAAATTT | T | C |
| 518 | GCCTGGAGAATCCCAGGGACCGGGGAGCCTGGTGGGCTGCCATCTATGGGGTCGCACAGnGTCGGACACGACTGAATCGACTTAGCAGCAGCAGCAGTAGCTACATAACATCCAACTAA | C | A |
| 519 | TCTCCTCCGGAGGCCAAGAATCCTGGCGTCTTTCATGGCTCAGCAACAACCTTTCAATAnAAGTACTATTATCAGCCCCATTTTATAAATATTGAGGCTTAGAGAGCCTAAATGAGTTG | T | G |
| 520 | ACCCAAATGAAGGGATACAGGGGCTGGCATGGGAAATAGTCAAGACAATAGGGCCAGGCnGCAAAGCACGGAGTGCCTAGAAAAAGCGTAGTTAGACCCCCGGCTCAGAAGGCGCACAG | T | G |
| 521 | CCCCGGCTCAGAAGGCGCACAGAACCCAAAATCCAGCGCCCACTGCGCCGACAGTGGACnCCAAGACCTAGAGACACACTAGGGCTGCTCAAGGGCGCACAGCGCAGCGGCGCCGGTCC | G | A |
| 522 | CGCCCACTGCGCCGACAGTGGACGCCAAGACCTAGAGACACACTAGGGCTGCTCAAGGGnGCACAGCGCAGCGGCGCCGGTCCCAGAGGCAGCTTCTCCAACCCCACCAGATCGATCCT | C | A |
| 523 | CACTGCCCCGTTCCGGAAGCCCTTAAGCATGGCCAGTGCTGCGTGGTAGCGGCGCTTGCnCAATAGTGCGTTGATCGCAAAGAGCAGAGCCCGCAGCTGCGGTGGGGCGACCATAATGC | T | G |
| 524 | AGGATGTGTCTGCTGTCACTCATACGAGCAGGGCCGATCACAGAGCTCACTCAACTCACnAGGGGAATGGGACGCAAGCACTCTCCCGAAGAGCGGTCTGATTGGAAGAGAGGGAAAGC | G | C |
| 525 | AGGAGTACATCAAAGCTGTATACTGTCACCCTGCTGATTTAACTTATATGCAGAGTACAnCATGAGAAACGCTGGGCTGGAAGAAGCACAAGCTGGAATCAAGATTGCCAGAAGAAATA | T | C |
| 526 | GTCTTTTCTGGCAATTTGATCTCTGGTTCCTCTGCCTTTTCTAAAACCAGCTTGAACATnGGGAAGTTCACGGTTCATGTACTGTTGAAGCCAAGATTGGAGAATTTTGAGCATTACTT | C | T |
| 527 | TTTTCTAATGAGTCAGCTCTTCGCACCAAGTGGCCATAGTATTGGAACTTCAGCTTCAGnATCAGTCCTTCCAATGAATATTCAGGGTTGATTTCCTTTAAGATGGACTCATTTGATCT | C | T |
| 528 | TGGATGGGGAAGCTGATGTGTTCAGCTGGGACCCACAGCCTGACTTTGGGGTTTCATAGnCATAGTCCAGTTCTTTTGACTCGTACTTTTATTCTTTTTTTTAAAAATTACTATATTTT | C | T |
| 529 | AATAGATGCCACCTCTCAATGGGAAAAACTGCAGGTCACACTGGAAAGGGCCTGGGTACnAGAAGAAGTAAAGAGTCGAGGCCATTCAGTCAGTGTACAGGATACCTTGCTTCATTGCA | C | T |
| 530 | AAGGCCTGACCTCGGGGTTAGTGATGCTCTATGAACGTGCCTCTGCAAGTTCTCTGCTCnCTCAGTACTAACCATGACACCCCTTGTCCCAAGAACATGCAAGGAGCCCCTCTGGGCCT | G | A |
| 531 | AGCTCAAGTCCCACATGACCCAGCACAAGAATGAGCAGGTAGGTGAGGGGGCGGGAGCCnGGGTGGGGCACAACCACCCAAGTCATGAGCCTTGGCTGATGCTGGGGTCTTGAACACGG | G | A |
| 532 | GGACTGCAGGTGAGGAGGCTGGGGCAATATTGGGGTTCTCGTAGCTAAATCTGTCCTAGnTTGGCATATGGGAGACGGGCCCAGAGAGGGGAAGGCCCACGGTCAAGGGTCACTCAAAG | G | A |
| 533 | TTTTCATGTTCCTGGCCCACTTTACTCATTCATCTTATTTATAGACTCTGGGTATATGAnTTTGACCCTGGTGGCTCAGATGTAAAGAATCTGCCTGCAATGCAGGAGACCTAGGTTTG | C | T |
| 534 | ACTTCCCTGGTGGTCCTGTGGTTAAAACGCCACGCTTCCACTGCAGTGAGCATAGTCTGnATCCCTGGTTGAGAACTAAGATCCCACATGCCGATGATGTGGCCAAAAGAAGGGGATGG | G | A |
| 535 | TGCAGCTGAAGAAGCTGATGGTCAGGGACAGGAGGATACTTTTACCAGCAAGCAGAGGTnGAATGTGAAATCAGGTCACCTGATCCCAAACTTAGAGCTTTTTTTTACTAGACCATATC | G | T |
| 536 | CTTTTATCAGCTACTTGTACGTGTGTGTGTCATCTGCACTGAACTGTGTGCCCTTGTAGnGTCTCATCCATCTTTCTACCTCTTAGTGTGCCTGAGACTATAGCCGGGAAACCAAGGAA | C | T |
| 537 | AAAGGAGATCAGTCCTGGGTGTTCATTGGAGGGACTGATGTTGAAGCTGAAATCCCAATnCTTTGGCCACCTGATGCGGAGAGCTGATTCATTTGAAAAGACCCTGATGCTGGGAAAGA | A | T |
| 538 | TCTGAGCGCCTACTGCACACTGCCAGGAAAGATGGGCTTGTGTCAGTGCGGGGAGCCTGnCTTCTCCCTGTCCCTGGCCATGCAGACCCTCCTTGCTACCTCTCAGCTCTCAGAACAGC | C | G |
| 539 | GAGCCGTATTCATCGTGTCCCTGAGGCCTGGGCTTCAGATGAACAGACCCTCTGCACAAnGGGAAGTTGAGGCAGGACAGAGTCATGCCACTTACCCTAAAAAATACCTGCTACTGAAA | G | T |
| 540 | AGAGTCCACATGCCAAGGGACAACCATCACATGCCAAGTATCACAACCATTGAACCCTGnGGGCTCTAGAGCCTGTGCTCTGCAACAAGAGAAGACACTGCAAGGAGAAGCATGTGCCC | T | C |
| 541 | CCATGTCTTGACTGTTTGCAGCCCTGTGGACTGTAGCATGCCAGGCTCCTCTGTCCACTnTCTCCCAAAGTTTGCTCAGATTCATGTCCATTGAGTCAATGATGCTATCTAACCATCTC | G | A |
| 542 | TGCAGTTTGCTAACACCTAAGCTACAGTGTGAAGAAAGAGCTTAAGTCATGGAGTTATTnGAACCAGTTGTAGGTCGTACACTGTAATTTTAACAATGCTACTGCAAGAGCTATCTGGT | G | A |
| 543 | ACCAAAACATAAAACAGAAGCAATATTATTACAAATAAAGGCTTTAAAAATGGTTCACAnAAAAAAAAAATCTTAAGAAAATAAAGGGAATATATTGGCTTGAGTGTGTGCTGAGTCAC | T | TA |
| 544 | CCACCATTCTCAGTATGGGTTTCATCATTAATTATTAAGGTCATCTCACAGTCCAAGAGnACTGCCTGAATTCCAGTCAGAAAATGGATTTTCTCTCTTTCTAGGCAGAAGGAAAAAAA | G | A |
| 545 | ACTAGTGATTTGTATGGTTTCCTTATTCTTTTAAGTTTTCTATCCACTTTATCTTACCTnGTTAGCTTTTGATGGAGTTTTGTTTTTGTTTTGAAATGTTTTTAAGCTTTTATTTAGAA | C | T |
| 546 | CCAGGTTTTCCACATCAGTGCCAACCGTTGTTACTCTGTGTGTATGTGTGGTGGTGGTGnTCAGTTGCTAAGTCGTGTCCAACTCTGCGACCCCATAGACTGCAGCATGACAGGCTTCC | T | G |
| 547 | TTGCTATTGAAGAAACAGAAGGCTTTGGACAAGAGTGAATTCTGAGAGCCTGCACCACCnAAGGACAAGAATTTATTTGCAACGTTTGTCCTTTTCCTTCTTTGCCTATTGCACACTTG | A | G |
| 548 | AAGGTGATAACTGCTCTGGGGAAAATTAAGCAAGTACTAGGAGGGAGGATAGTCACTGGnGTGGGAACTGCAATATTCAGTAAGATCATTCAGGAAGGCTTCCCTTGAGGAGACCTTGG | G | T |
| 549 | TCCTGGGGGATTCCCAGAATTGTGATAGAAGGCCGCCAGTGCAGTTGAGGCCAAGAGGGnAAGAGTGAGGTAGATAAACCTAGAGAAGCAACAGGGAGGAAGATCCTACAGGCCTCACG | C | T |
| 550 | AGAGTCTATTTTGGGGAAGATGATGGGTTCATCTTGAGTAGGTCAAGTCTGAGATGTCTnCTAGACTTCCATCTAGAGACATAACACATGCCAGGCCTTTGGATTCAGGGAAGATGCAG | G | T |
| 551 | GGGAGACGGCGCCCCTCCGCGCCCGCCCCTGCGCAGGCTGTGACGTCACAGGACTGTGAnGCGCCCTGGGAGCCCCCGCCCCTTCGCGGCGCGGGGCCGGGAACACAGGCTCTCTGCGC | C | T |
| 552 | AGGTGGGGTATGGGACCGGGAGCTGGGTCCCTGTTCTGGGGAGGTTGGGAAAGGTCAAGnTCAAGGCTGGGGTCAGTGTCCAGTCAGAGGTAACTGCCTCTAGAAGGGCCTGGGATGGG | G | T |
| 553 | CCATTCTGTCACCGCAGGAACTCATCTAGAAGGCTGATGGAACCCATCTTACAGACGAGnAAACTGAGGCACCAAGAGTTAGTTGGCCTAAGGCTGTACTGGGAGAGCTGGACTGGTGG | G | C |
| 554 | CATGGCCAACAGCCCTGGGCTGCAGTGTCCTGGAGAGGCAGGGAACCCCAGGCATGTCCnGGTTCCCCAGGTCTCATTGTGTTGGGGATGCTGACACCTGACAGCCACGACTGGGAGAA | C | T |
| 555 | GTGCTATGAAGGGAACACACAGAATGCCAAGACAGAGACCGTGGGGCAGGGAAACCTCAnAGATCTCTCTGGGGAGGTGACCATTAACTGCTATGAACTTCTAAATGTCCATTTGCACT | CAGA | C |
| 556 | CTGAGTCTGCTGCACCAGCCAGAAGAGAATAAATGTGTCTGCAGTTCCTATGGCTCCTCnCGTCTTCTTCCACCCTCTTAGCTTGCGCCTTGCCTATCCTGGGTTCAACTAATAGTTCG | G | A |
| 557 | TAATATGCAATTTATATATTCTATATATAATACAGAATACATATCTTATATATTCTATAnTATATATATATTCTATATTATATTATATAACCTAAACCATCCCTCCCCACTCCATCTAT | TTA | T |
| 558 | CTGCCAGAGCAACTCCGTCTTAGGGGAAAGAGGACCAACGTCCCCAGACTCATCCCATCnCAAACTAGGGTATGTTCACACGTTCTGTGCCTTTGGGACTTGCCAAGGGGCCCCAGGCT | A | AC |
| 559 | TCCGCAGCAAGAGACCACCGCAATGACAAGCTAGCGCATCCTAACTGGAGTGTAGCCCCnACTCGCCACAAATAAAGAAAAGCCTGTATAGCAACAGAGACCCAGCAGAGGCAAAAGTA | C | G |
| 560 | GGAGGTCAGGTATCTTCACACCTTCTGTTCCCACAGCTCTGTGGCCCTTGGTGGGGGTTnGGGGGGGGGTGTCACAAGATGGCGGTGGCCCCCAGGAGCTATCTGTGGCCTTTCACACG | G | GA |
| 561 | GAGGACAGGTGTGCTGGGTTCCCTGCTTCTTTTAGCCCAGACCCAGGCCTGACCAGAACnCGGGTAGGACATATGGACCACAGACAGCTGGAGAGCCTGGCTGAGGAAGGCTGCCAGAA | G | A |
| 562 | ACATTAAAAACATACATATGGGGTTGAAACGGATGTCAATTCAGTTCAGTCGCTCAGTCnTATCCAACTCTTCGCGACCCCATGAATCGCAGCACGCCAGGCCTCCCTCCCTGTCCATC | G | A |
| 563 | ACGTACTCCTTTTCCTATTTGGAACCAGTCTGTTGTTCCATGTCCAGTTCTAACTGTTGnTTCCTGACCTGCATACAGATTTCTCAAGAGGCAGCTCAGGTGGAGTGGTATTCCCATCT | C | G |
| 564 | GAAGCCATAGACTGCAGGAATGAAATCCCAGGAACTAAGAAGGAAGAAGGAGAGGCAAAnGACTACCACTAGACTTCCCATATACATCATGGCGTACTCCCAAAAGAAGATCCAAAAGG | C | T |
| 565 | AATATATAGAACAAACATGGACAGAGTTACAGAGGAAAAATACACAAATCCACCATCAGnGGATAACTGGTAGATCTAATAGACAAAAATCATAATGAAATAGAAATATGAATAGCATA | C | T |
| 566 | TCCAGATTTCTGGTGAGCAGTGTGGTCTTGCATAAAAAAACTATAAAATTTCTCACACTnTTGGTGCTAAAAATTTAGCTGGAAGAACACACCTTAAGGAAATTAAATGTCTATACTAC | T | G |
| 567 | CATGCCCCAACTAGAAGTTCCCATGCTGCAAGTAAAGATCCCACATGTAGCTATGAAGAnAGAAGATCCTGTGCACCACAACTAAGTCCTGGCTCAGCCAAATAATTAATTAATTAATT | T | C |
| 568 | AGCTACAATTAGCCACCAGGTCTGTCTCACTAAAAAAAATGCACAACCTAAAAAGTTGAnATTTATATTTTATTTGGTGGACAAGACTGAGGACTTAAGCTTGAGACAAACCATCTCAG | G | C |
| 569 | TGGAGGCTGGAACCCAGATCAGCATCGAGTGAAAAAGCATCTGTGAAGGGTTGATTGTGnGTGTGCCTGTGTGAGGCTTTGTCCCAAAAGAGACAGAATGGCTCTGTCTCTTTTCCTGG | C | T |
| 570 | TGGTCCAGTGGCTAAGATTCTGAGCTCCCAATGCAGAGGGCCCAAGTTCGATCCCTGGTnAGGGAACTAGATCCCACATACTGCAACGAAGACCTGGTGCAGGCAATTAAGTAAATAAA | C | T |
| 571 | CTTTGGGAGCTTCTCCTTTGTTATGATATGAATGATCAAGTGGTGTGTCTAAGCAACACnTGTGGCTGGGAAGCATGGTTAGCAGTGGGGGAATAATCAACCTTAGGCTCCACCCTCCA | T | A |
| 572 | ACTAGAAGAACTAAAATATACACCATAAATACTGCTGAATTGTAGGAATACTGGCTAACnGATAACATTCTTCTTCATAATTCTCTGATCTGAATTTCAATTTATCTTTAGTAAACTCT | G | A |
| 573 | CAAAAAACCAGTCAAAAGGATGCCTCTTCTGAGAAGTCTTCCATGACCTCAGTCCCCAAnAGGAGTTGCCCCTTCTGCCTTTTGGGTTCAGAGTCTGTGTCTGGCATTCTAGAGCACCC | G | A |
| 574 | TAATGGAGGTGCCTCTTTCTCCTGCTAAAACAAAAGCCTCAGCAATACTGTCCTGGTGTnTATAGGGTCAATGGTCAGGCGACTCAGGGAACAAACACCTATAAGGTATCTTTGGTCTG | T | C |
| 575 | CTACAGTTGTATTTCATCTTGTTAGGTTATGTGGGACAGCACAGTATAAAAGACAAAGCnTGAGTTTTGTGGTCAGAGCCCATCTGCCTTTCTCTCACTTGAGGATCAAACAGCTACAC | G | C |
| 576 | TTAAAATAATCACAACTTCTACCTTACATTTTAAAAAGTTTTTTATTCATTTATTTATTnGACTGCTCTGTTCTGGTGTGGCTTGTGGCATTTTAGTTCCCTGACCGGGGAATGAACTG | C | T |
| 577 | ATTTTATGAGGACCTAATATAACAAAGACTAATTTGAGCAAAATACACACACAAATACAnATACACGTGCACATACACACACTTTCAGAGGTTATATTTGAAACTTTTTACTTACTAAC | TATAC | T |
| 578 | TATCTTTAGGGTAGCTATCAAATTCAACCAGGGAATCCAGACTTCTTTTTTTGTGAATAnTGCAACGGTGGCTCCAACAATCCATAACACCTTGCAAGAAATTGTAGACTGGCTAATGA | C | T |
| 579 | CCCACCAGCCATCAGTTTTCAATTTCTAATTAAGTCTAGTCAGTTTCTAATTAAGTCTAnTAAGTCTAAGTCTAACCTGGATTGAGTTAAATCCAAGTATAACTTACAAAACTGGTAGA | A | G |
| 580 | CTGCCTGCAATGCAGGAGACCCAGGTTCAATCCCTGGGTGGGGAAGATCCCTTGGCGAAnGGAACGGCTACCCGCTCCAGTATTCTTGCCTGGAGAATCCCACGAACAGAGGAGCCTGG | C | T |
| 581 | AATCGGAAGAGAAGCACTTCCTGGCTTCGGACCCAAATCACAGAGGTTTCCTCTCCCCTnTCCATCAACCTCATGCCTTTCCCTCCACCCCCAGGCCCCACTGACCTAGATCATGGGTC | C | T |
| 582 | AGAGTTAGGATTCCAAGCCTAGACTGTCTCCAAAGCATATACTTTGTTCTACTTCTTGGnAATGGCATTCAGGCTATTTCTCCTAAGGTTTGGCAAAGACGTAACTGTCTTCCTGTGAG | G | A |
| 583 | TAGCTGGCATCTATTGATCTCTCAGTGCCAGCCAGACACTGGGGAAGGGCTCAGTAATCnCTCAAGGAGGAAGTTACAACCATCCCTGAAGTATGAAGAAATTGAGGCTCGGGATGAAC | C | T |
| 584 | TGGGTTTCCTGCATTGCAAGTGGATGCTTTTACCAACTGAGCCACCAGGGAAGTGACTCnATTCAGTAGGTTGTCACTTTGTTTTATCAATGGTTTCCTTTGCAATGCGAAAGCTTGTA | C | T |
| 585 | CTCGTCCCTGTACCTGTTGAAGAAACTGGGTCATTTGTCCTGAGTATAGGCATTAGACTnAACTAGATTCGGGTTCACTTTTTTTTATATATAATTGTTTCTTTTTTATTGGAGTATAG | C | G |
| 586 | GCATCTCTGGCTCCCAGCCTAGAGGCAAATAGACAAGTAAGGTGGCAACAAAGGCAGTAnGGGAAGGCACTTGGGAGAGAGCATCTGAGCAGAGGGCCCTGTGAGAGGGGGTTGTCAGG | A | G |
| 587 | CTAGGTCTCAGCTTCCATATTTATATTATAGGAAAGATGATACCTACGGTCCTAGAATTnTATACCTACCAGAATCTATCCTGTAGGGTATTGTGAGTGAGGATGAATGATGAGAAGTA | G | A |
| 588 | TTTAGGGCTGGCATGCTGAGGACTAAATCCTCACAAGGCCTGAGTGCCACACAGAGTGTnTTCTATCCAGTCACATCCTTCTGTCTGCTTCCCTTTATTGCTCCTGAGTACCTTCCGTG | G | A |
| 589 | GAGATGGCTGTCTGTGGAAGTTAGTCATTTACTCTCTTGACAAGGCATTCAAACACATCnGCAAGCCCGTAATCACCCTGAAAAGAAGCTGATGGGGTACAGTAAGGTCCTGGGAAGAT | C | T |
| 590 | TGTATACTGTCACCCTGCTTATTTAACTTCTATGCTGGGCTGGATGAATCACCAGCTGGnATTAAGATTAAGATATCAACAACCTCATATATGCAGATGATACCACCGTAATGGTAGAA | A | G |
| 591 | ATGAGGAAACTAAGACCCAGGGAGAGGCTGCAACTTAAGGTAGTTTTAGGACAAAAAAAnTTTTTTTAAAGTTTCCATTTACAAGATGAGGAGAAAATTTACCCAAGGAGCATGAACAT | A | T |
| 592 | GGCAGAATATTGAAAGCATGCTCCAAAATGCACACAGAGCCCCTCAACAAATACTGAGAnACTTACTGGTTCCACATTTAAGGAAATCTCCATCCAATCATTACTTGACCACTAAGCTA | G | T |
| 593 | TTGAAACAGAGTGCAGAGGTACTGATAGGAGATGAGGTCAGATCATGCCAGCACCACAAnAAGTATGGCCTCACGAGCCAGAGAAAAGAGGGTGGATTTTATTTTTAGTGCAGTGGGGA | T | G |
| 594 | CACCCACTGCCATCCATGGGCTCAGGCCGCTGAGCTGGGGTTCCCAGTTCCTCCTCCTCnTCCACTGGCCCGACTCCCATGTCTGACTCCAGCAGGAGCTGTTTCTCCCCTTCTGTGTC | C | G |
| 595 | GTAGGGATAGGTTGCTGGGTGGAGGGAAGACTTTTCCAGAAATCAGCTTGCCAATTTTCnCCTTCCCTCCTCTCTTCTGTCACTCGTGGGAGACGACCAGGGGCACAGAACACGGCAGC | C | T |
| 596 | GACACAGTCAGTGTGAAGTGGGTTCCAAGCAGTGGGCCATAGGAGAGGGCTGAGGGGTCnCAGGCCTTACCTGAGCCCTGGGAGGGAGGCTGGCAGGCCGGCAGACCGGTCATATGGGG | T | C |
| 597 | GTGGCTGAGGGACATGCTAGCCAGCTCGGCTCAAAAGTCCTGCAGCTGAGGGATGTCCCnGTACAAATCCAGGGTCTCGGGATTCGAGAAGGCCCCTCGGTGCTCCACCGTCTTCCCAG | G | A |
| 598 | ACAGGTGTGATGTGGGGCTGGGGGCAGAACTGAACTAACAGTTCTCACCCCCAAAGGCAnGCCTCCTAGTGGGGTCCACCCTACACCCAGTTCTGAGATGCAGCGGTGCGTGAGAAACA | T | C |
| 599 | GAGGAGGCAGGGATCAGAGCTTTAAATAAAGAGACTTCCCAGCTCAGGGGCAGGGTGCCnCTCCAAAGATGAGCGAAGAGGGCAGCCTGAGTGAGTGACGGGCCTGAAATTAATTATCC | A | G |
| 600 | GGTTAACACTCCACAGCTTCCAGTACAGGGGGCATGGGTTCGATCCCTGGTAGGGGAACnAAGATCCTACATGCCATGTGGTGTGGCCAAAAAAATAAAACCTCCAGGTCTCACCTCTC | T | C |
| 601 | CATGTTGCCATGAAGAATGTGTTCCTTCAGGTGCTGGATGAGCGTCCCCTGGGGAGAAGnGAGTGAGCCAAGCAGCTTCCTCCGGGGCACAAAGGCACGCGTGGAATAGACACTGGGCC | T | C |
| 602 | ATCCAACCATCTCATCCCCTTCTCCTGCCTTCAATCTTTCCCAGCATGAGGGTGTTTTCnAATGAGTCAGCTCCTTGCCTTAGGTGACCAAAGTATGAATCTTAAGGAGTCAAATGAAT | T | C |
| 603 | TTTAGAGATCTCTTCTGACTCGTGAAGGTCACAGCAAGAAAGTGGGATCAAGGTCCCACnAGAGTTTCTATGACAGAAATCCTGCCAGACAATGATCCTGTTCTCACTGATTGCCTATA | A | G |
| 604 | GAAGAAAGTACTATTATTCCCATTTTACAGATGGGGAAACCCAGGCACAGCAAGGTTGAnCGCTAGCCCAGGGCCGCACAGCTGACATACGACCCAGAGCAGTCTGGCTGGAAGCCACA | T | A |
| 605 | CTCCTGCTAATGCAGGGGTCACGGGTTCAATCCCTGGTCCAGGAAGATCCATCATGCTGnGGAGCAACTTAGCTCATGCACCTCAGCTATCGAGCCCACACACCCTAGAGCCCTTACTC | A | C |
| 606 | CTAGGACCAGGGAGGAGGCCAAGAGCCCAGGCCATGGGGCATCTATGACAGCACAGATCnGGGCAAAGTAGGAAGAGTCAGCACAGCTGTGATGAGGGGAAGGGGAGGCCTTAGGGCTC | C | T |
| 607 | GAAGCCAATGACCACATTGGGAGGAACTCAAGCAGTCCTAGGTGGCAAGTAGCGGAGGCnCCCTGCCCACAACCAGCAATGACTCACTGCCATGCGAGCCAGCCCCTGAATAGTGTGGC | C | A |
| 608 | GCAGAGCGCTCAGCCTGATGGCCTTCACGCCCAGCCAATGATGTCAGTCCTCTCATTATnTCCATTTTACAGATGAGGGAAACCAAGGCTCGGGAGATCCAGTGACTTCCCTCTGACCA | G | A |
| 609 | AGGACCGGGAGCAGGCCGGGGACCGAGCCGGGGGCGGCAGCAGAGGCCGGGAGCGGGACnCCGGGCGAGCGGCGAGGCAGACGTGGAGCAACTGCACGCGGAGCAGCCGCCCCGCCCGA | T | C |
| 610 | GTGTAGGCTTTGCAGATACATCACCGAGCAAAGCGTTAATTCCTGCCCTCAGTCAACAGnAGATAAGGGGTAGGGGACAGGTGTGGGGAGGAGCGGGGTCAATCAACAAATACATTATA | A | G |
| 611 | TTATGGGTGGAGAAGGCAATGGCAACCCACTCCAGTATTCTTGCCTGGAGAATCCCAGGnATGGAGGAGCCTGGTGGGCTGCCGTCTATGGGGTCACACAGAGTTGGACACAACTGACG | G | A |
| 612 | TACACCTGACAGCTACTTGGTTCATTCTTATTTCTTTCAGCTCAGAGCCCCCCACCCCCnCCCCCATAGAGGCACAGGACCTGTAGGGGAAGCCAAGGGAGAAGTAAGCTACAGTCAAA | A | C |
| 613 | ACCAAGAATCTTAAACTTTCTGTGGGTAACCCCTCTTAGGTCTCTGAAATTCCCATCTTnCCCATCTTACCAGTTGCTTTCTTTTTTCTTTTTTGGCTGCACTGGCTCATGGTTGCTAA | T | C |
| 614 | GGCCTGAGGCATGCTTGGCACAGTCAAAAGGTAGTGACCATACGTATAGGGCCTTGGGAnGCTCTAGGGTAAGGACTTCAGATTTCATTCAAAGTGCAATCAGAAGCCACTTGGAAAGT | G | A |
| 615 | CAAAAGCTTTTCAGACAAGCAAAAGCTGAGAGAATTCTGCACCACCTACATGTCTATATnCTTAAAGAGATGTGAATACCAGACCACCTGACCTGCCAGTTGAGAAATCTGTATCCTGG | C | T |
| 616 | CCATGTGCCACAACTAAAGATCCTGCATGCTGCAACTAAGGCCCAGTGCAGCCAAATAAnTAAGTATTTTTTAGGAAAGGAAGATGAAGCCATAAAATCTGTTTGTTTGATTTTTTAAG | C | A |
| 617 | GCAAGCTCCGGGAGTTGGTGAAGGACAGGGAGGCCTGGCATGCAGCAGTCCATGGGGTCnCAAAGAGTCGGACACGACTGAGCAACTAAACTGAGCTGACTCGAAGAAAGGCAAATACA | A | G |
| 618 | CACTCATCTCACATGCTAGTAAAGTAATGCTTAAAATTCTCCAAGCCAGGCAAAACATGnACCATGAACTTCCAGATGTTTAAGCTGGTTTTAGAAAAGGCAGAGGAACCAGAGATCAA | G | A |
| 619 | GTTAGAGGAAACTTTTGGGATTAAAGATTTTGATTCGATATGTACTTATAAGAGAGTATnTTAAGTTGCATGATAAAAATGCAACTTTAAACAGAAAACCTTGTGAATCTGTGACTGTA | G | A |
| 620 | ACAATGGGCATGCTGCTGCTACTGCTGCTAAGTCGCTTCAGTCGTGTCCGACTCTGTGCnACCCCATAGACGGCAACCCACCAGGCTCCCCCGTCCCTGGGATTCTCCAGGCAAGAACA | A | G |
| 621 | GTCGCTTCAGTCGTGTCCGACTCTGTGCGACCCCATAGACGGCAACCCACCAGGCTCCCnCGTCCCTGGGATTCTCCAGGCAAGAACACTGGAGTGGGTTGCCATTTCCTTCTCCAGTG | A | C |
| 622 | CAACCACTTGCATAAAAGTAATAGAAACACAATGGGGACTTTTTTTTTTCTTTTTTTTTnAATTTTATTTAATTTTTAAACTTTACATAATTGTATTAGTTTTGCCAAATATCAAAATG | T | A |
| 623 | ACAAATAATAAATGCTGGAGAGGGTGTGGAGAAAAGGGAACCCTCTTACACTGTTGGTGnGAATGCAAACTAGTACAGCCACTATGGAGAACAGTGTGGAGATTCCTTAAAAAACTGGA | G | A |
| 624 | CAAATAATAAATGCTGGAGAGGGTGTGGAGAAAAGGGAACCCTCTTACACTGTTGGTGGnAATGCAAACTAGTACAGCCACTATGGAGAACAGTGTGGAGATTCCTTAAAAAACTGGAA | G | A |
| 625 | AGACTCTTGAGAGTCCCTTGGACTGCAAGGAGATCCAACCAGTCCATCCTAAAGGAGATnAGTCCTGGGTGTTCTTTGGAAGGAATGATGCTACAGCTGAAACTCCAGTACTTTGGCCA | C | T |
| 626 | TCTATACCCTGTGTGAAAACAGATCTGTCTCTAAGTACCTGCTGCTGCTGCTGCTAAGTnGCTTCAGTCGTGTCCAACTCTGTGCGACCCCATAGATGGCAGCCCACCAGGCTCCTGCG | T | C |
| 627 | ATCCCCCCACCCTTTTCTTTTCCTGGCTGCACCATGTAGTACGTGAGATCTCCGTTCCCnGATAAAGGATTGAACCCGCACCCTCAGCAGTGAAAGCTCAGAGTCCTAACCGCTAGACC | T | C |
| 628 | GAGTTGAATTCTCTGTCACCGCATGGTATCTGCACATTGCTAGCTGGTGGGGGGAAGCCnACACACACACACACACACTTTGGAATTTGGTCCAGGAACCTTTTTCAGTCGCACGCCTA | TACACA | T |
| | | CACACA | |
| | | CACACA | |
| | | CACACA | |
| | | CACACA | |
| | | C | |
| 629 | TTTTTAACATGGCAATTAGAACGTTTAACGTCACATATGTGGCTGGCATTCTATTTCTAnGGAACCACTCCAGGCTAAGGACTCTCCATTTTAACCAGTACCTTGAAACATTCTGAAGC | C | T |
| 630 | GAATACGGTGGTTCAAAGCCAGAGGGTGGGTTCCCAGGGATGGAGGGGATGACAGTGGGnGCGTACCAGGGTATGGGTTTGGGCCAGCAGCCAGCTCAACTCCAAGGACTCGGTCTCTG | T | C |
| 631 | GGCTCAAGTCGCTCCAGCTGGTCCCTGGCTCATGGACTGCACCCCTCCTTCTCTGTCTCnGTTTTCACATTCACATGGCCATCATCTCTGTGTGTCTGTGTCTCTGTCCAAATTTCCCT | T | C |
| 632 | CCTTGCTCACGGATCTCAGAGGGGAGATGAGTAACAAACACACACACGTGGGAGAAGGAnAGGAGGTGGAAGAATGTGAGTGTGAGAACTGAGCATGAGGTTCCTCACACAAGGTGGTT | C | T |
| 633 | AACCAAGCATGGGAAAGACAGGAGCAGGCAAAGGGCTGGGAAGATCGTCCCCAGCAGGGnACAGAGACTGGAAGGCGTCAAGCTGGGTATGTCCAGGGGCTAGGCAGCATCCTTGGTGT | AAC | A |
| 634 | GGCTGGGAAGATCGTCCCCAGCAGGGAACACAGAGACTGGAAGGCGTCAAGCTGGGTATnTCCAGGGGCTAGGCAGCATCCTTGGTGTGAACTTTGTCAATTGAAAGTGCATCAGGAAA | A | G |
| 635 | GCTAGGCAGCATCCTTGGTGTGAACTTTGTCAATTGAAAGTGCATCAGGAAATCACTGGnCAGTTTTAACGAGAGTGACTTAATCTGACTTTTACATTTTGTTTTTTTAAAATCACTAA | A | G |
| 636 | TGTGTTTGTTAATCAGGTGGTTCTGAAAAGCCTCTAAAAATGTAGGCTGGTTGCCAAGGnAACCAACTTGGTTATTAGAGGGTTGAGCCTTTCGATTGACCCTCCTGACCTCTTGGGAG | A | G |
| 637 | TGGGACTAAGCCTTTGACCCGTAGGATCAGATGCTACCTCCAGGGAGGCAGGGTCAGAAnTGAGTTCAATCTGGGCCTGGAGGTTTTGCAAACAACGTGGTTCACACTGATATATGCTT | A | C |
| 638 | CCCTTCTCCAGGGGATCTTCCTGACCCAGGGATTGAAGCCAGGTCTCCTGCATTGTAGGnGGATTCTTTACCATCTGAGCCAACAGGCAAGTCTAGGGACAGACAAGTTGGCAATACCC | T | C |
| 639 | AACCCCAGGGCTTGGCCAAATCGCCCTGTGGATTAATGGAGCATAGGACTAGCTTGGGGnGCCAAGAACCAAGTGAATATGGTCAAATCTCCCTCCTGGACTTCCCTGGTGGTCCAGTG | T | C |
| 640 | GAAGCCCATGAGCCCTAGAGCCCGTACTCTGCAAAAAGAGAAGCCACTGCAATGAGAAGnCTGCACACTGCAGCTAGAGTAGCCCCAGCTCTCGAAACTACAGAAGAGCTGCCTGCAGC | G | C |
| 641 | CCAGAAGCCCTCTGCAAAAGTGCTCACTACACTTCTGATTACTCCCAGAGCCCTCTGCTnACTCTTGTCAAAGTCCCTGCCTGAATTCTCATTTCTAAATGGTGAAGGTGTTGCAACCA | T | G |
| 642 | CCTGAATTCTCATTTCTAAATGGTGAAGGTGTTGCAACCAACCTATTTCACCCAGAGGCnGGGTGCTGCTGCATGGGAGCAACTCTAAAGGGCGAGAACGGCGGTTCTCACAGTTGAGT | T | C |
| 643 | CAACCAACCTATTTCACCCAGAGGCCGGGTGCTGCTGCATGGGAGCAACTCTAAAGGGCnAGAACGGCGGTTCTCACAGTTGAGTCCCATCGTCACATCTCGGGCACGTGCAAACGACA | A | G |
| 644 | AACCTATTTCACCCAGAGGCCGGGTGCTGCTGCATGGGAGCAACTCTAAAGGGCGAGAAnGGCGGTTCTCACAGTTGAGTCCCATCGTCACATCTCGGGCACGTGCAAACGACAGAGAC | T | C |
| 645 | ACAGGGACTTCCTGGTGGTCCAGCAGATAAGACGACTCTGAGCTGCCAATGCAGGGGGGnCAGGGTTTGATTCCTGGTCAGGGAACTAGGATCCTGCCTGCCCTGCAGTGTGACCAAAA | A | C |
| 646 | TAGCACTCAAGCTTAGTTGCCCTGTGGCATGTGGAATCTTATTGGACCAGGGGTGGAACnCATGTCCTCTGCATTGGCAGGTGGATTCTTAACCACTGGAGCACCAGGGAAGTCCTCAA | T | C |
| 647 | GATGTATCATTTACATACTATAAGGTTCTCGTCTTTAAAATGCATGATTCAGTGTTGTTnTTTTTTTTTAAAAAATCCCCCATAATTGAATTTTAGAACATCTTCATCTCCCCAAAAGG | G | GT |
| 648 | CCCTGGGATTCTCCAGGCAAGAACACTGGAGTGGGTTGCCATTTCCTTCCCCAATGCATnAAAGTGAAAAGTGAAAAGTGAAAGTGAAATCGCTCAGTCATGTCCGACTCTTAGCGACC | G | GAAAGT |
| | | | GA |
| 649 | TGTCCGACTCTTAGCGACCCCATGGACTGCAGCCTACCAGGCTCCTCTGTCCATGGGAGnTTCCAGGCAAGAGTACTGGAGTGGGTTGCCAGTGCCTTCTCCCTCTCAGCCTAGGCAAC | C | T |
| 650 | GAGTGGGTTGCCAGTGCCTTCTCCCTCTCAGCCTAGGCAACCACTAATCTTCTTCCCGTnTCCACGGTTTTGCCTTTCCTGGACTTTCTGTACAAATGAAATCAGGCAATCCGTGACCT | T | C |
| 651 | GTTTTGCCTTTCCTGGACTTTCTGTACAAATGAAATCAGGCAATCCGTGACCTTTTGTGnCTGGCTTCTTGCACTCAAAATAATAATGTCTGGCTTCACCCATGTGGCTGCGTGAATCA | A | C |
| 652 | TCTTGCCAAATAGCACTCCCTTCCAGGGTTCTCCACGCTTTGTTCCTCCTCTCGTTATTnTTTAAAAGCATCACAGTTTGTGCCCATTTGTCCATAGGTAGCAATTTTTACATTTTGTT | C | T |
| 653 | CTATTTTGGGAAGAAGGGACATTACAGCTTCTGCTGCCGCTGACCTTCTCTGTGCCTTAnTTCTCAGCTGTAAAATGGGACTGAGGAAGAATGGACATTAGGGAACTTCCCTGTCCACC | GT | G |
| 654 | GCTCAGAGGAAAGGTGAGGCCCACCTTGGGGACCAGCCAGGGCAGGAAAGGGTGATACAnGGAAAGGCTCATCGGGGGCCTTTTGGACCGGGTGGGTGGGAACACTCTGCCCCCTGGGG | C | T |
| 655 | TGAGGCCCACCTTGGGGACCAGCCAGGGCAGGAAAGGGTGATACATGGAAAGGCTCATCnGGGGCCTTTTGGACCGGGTGGGTGGGAACACTCTGCCCCCTGGGGCTGGCACCGCTCAC | A | G |
| 656 | CCTGGGGCTGGCACCGCTCACTGTGGCTTCTGCTCCAGCAAGTTAGTCCTTGGCTCCCTnTCTGTGTGAAGACACAGGAGCCCTGAGGGGATGCTGTGGGCAGCACTGTGTTCCTCCAC | C | G |
| 657 | GTCTGTGTGAAGACACAGGAGCCCTGAGGGGATGCTGTGGGCAGCACTGTGTTCCTCCAnATCTCAGTGAAGGTGAACTGAGGCCAATGTGGAGGACCTGGATCCAACAGGACTGGGGG | A | C |
| 658 | GTGTGAAGACACAGGAGCCCTGAGGGGATGCTGTGGGCAGCACTGTGTTCCTCCACATCnCAGTGAAGGTGAACTGAGGCCAATGTGGAGGACCTGGATCCAACAGGACTGGGGGCCCT | C | T |
| 659 | GCACTGTGTTCCTCCACATCTCAGTGAAGGTGAACTGAGGCCAATGTGGAGGACCTGGAnCCAACAGGACTGGGGGCCCTACAAGAAGAGATGGGGGGCTTCCCCACAGGTCCCCCAGT | C | T |
| 660 | GGACCTGGATCCAACAGGACTGGGGGCCCTACAAGAAGAGATGGGGGGCTTCCCCACAGnTCCCCCAGTGGTTAGAACTCGGTGTGGTCAGTGCTACAGGCGCAGGTTCAGTCCTGGTC | G | GT |
| 661 | GGGCTTCCCCACAGGTCCCCCAGTGGTTAGAACTCGGTGTGGTCAGTGCTACAGGCGCAnGTTCAGTCCTGGTCAGAGAACTAGGATCCCGCATGCTGAGCAGCGTGGCAAAAAAAAGG | C | G |
| 662 | CCCACAGGTCCCCCAGTGGTTAGAACTCGGTGTGGTCAGTGCTACAGGCGCAGGTTCAGnCTGGTCAGAGAACTAGGATCCCGCATGCTGAGCAGCGTGGCAAAAAAAAGGTTAACACT | TC | T |
| 663 | TGGTCAGTGCTACAGGCGCAGGTTCAGTCCTGGTCAGAGAACTAGGATCCCGCATGCTGnGCAGCGTGGCAAAAAAAAGGTTAACACTGCATTTGATGGGAAAAAAAATTTTTTTTAAA | T | A |
| 664 | AGGTTCAGTCCTGGTCAGAGAACTAGGATCCCGCATGCTGAGCAGCGTGGCAAAAAAAAnGTTAACACTGCATTTGATGGGAAAAAAAATTTTTTTTAAAAGAAAGAAAATGAACCCAC | A | G |
| 665 | TGAGCAGCGTGGCAAAAAAAAGGTTAACACTGCATTTGATGGGAAAAAAAATTTTTTTTnAAAGAAAGAAAATGAACCCACTCCAGTCTTCTTGCCCAGGAAATCCCATGGACGAAGGA | A | T |
| 666 | TTTTTAAAAGAAAGAAAATGAACCCACTCCAGTCTTCTTGCCCAGGAAATCCCATGGACnAAGGAGCCTGGTGAGCTACAGTCCAAGGGGCTGCAAAGAGTCAGGCACCACCATGCCCA | A | G |
| 667 | CTTGCCCAGGAAATCCCATGGACGAAGGAGCCTGGTGAGCTACAGTCCAAGGGGCTGCAnAGAGTCAGGCACCACCATGCCCATGTTTAACTGAATCTCTGCTGTACACCTGAAACTAA | G | A |
| 668 | GTTCAGTGAAGCCCAGAGCCTGAAGCAGTTCACAGAAATAGGTGAAAAGCCAAACCAACnGCACAGGTTGTATGTTTTTTCTACAACCTATAAAATATAGCCAAAATTTTATAGTAACT | A | G |
| 669 | ATTTTATAGTAACTATAAATGAAGTATAACCTATAAAAAGTGTGAATGTTGTATACCGCnAACAGCACTGTATAATCATATACTACTGTACATCAACTCTACCTCAATTCAAAAATGTA | A | G |
| 670 | GGTTCATCATACAATTCTTTCTCCCTTTGTGTCTCCTGGGCAATATTCAGGATGAACAGnTAAAATGAAAAAGATTCAAGTGAGCCAACTCAGGAGGCCCGCGGCCATGGCCAGTCCAA | A | C |
| 671 | GGCCATGGCCAGTCCAAGGGAGAAGACACAATTACTCCAGCTCAGCATTTCATCACCCCnTCCTAAATCTGGAGGGCATCCTAAACTCTCCTCACCCCCATTACCCCCAAGCATATTTA | G | A |
| 672 | GCTGGGTAACTGAGGTACCATGTGATATATGCTGAGGCCAAAAAGTAAGATAAACCACGnGGAGAAACCATGTCAGTCCTACTAGAATGGTTACACTCAAAAAGTGTAACCTACGTCAA | A | G |
| 673 | GAGGTACCATGTGATATATGCTGAGGCCAAAAAGTAAGATAAACCACGGGGAGAAACCAnGTCAGTCCTACTAGAATGGTTACACTCAAAAAGTGTAACCTACGTCAACAGGTGAATGG | T | C |
| 674 | CAACTCTGAGCTTCCACTGCACGGGGTGCAGGTCCTGGTTGGGGAACTAGGACCTGACAnGCTGTGAGGCAAGGTCAAAGAGAAAGACAACTACTGTATGATAACACTTTGTGGGGAAT | C | T |
| 675 | AGAGGCAGACTCACAGATACAGAGAACAAACTAGCGGTTACCAGCAGGGAAAGGAAAGAnGGGGGATGGACAAGATAGGGGTAGAGGCTAAGAGATACAACTACTATGTATAAAGTAAG | T | A |
| 676 | TGCTGATAGGAACGTCAAATGGTCTGAACAGAAACTCCTTGAGATGTCAGAGCTGCCATnTGCTGTGCCGTGTGCTTACTCACTCAGCTGCCCAACTCTTTGCGACCCCATGGACTGTC | A | T |
| 677 | AAACATTTTGGACCCTAACTTGATGTGACCTCCACATCTAACTCACAGTTTACAGGAAAnATGGGGGATGGAAGCAGAAAAATTAGAAACAGCAGAAGCTAAAGTCAGAATGTGGAACG | C | T |
| 678 | TACCCAGGTCAAGAAAAGGAACATTATCAGTTTTGCCAAAAGGCTCCCTTTGCCCTCTCnTGTTCACTACACCCCCTCCAAAATTTAGGGGATTCATTTTGCCTGTTTTTGAACTTCTG | T | C |
| 679 | TCTCCTCCTGTCTTCAGTCTTTCTCAGCATCAGGGTCTTTTTCAATGTGTCAATTCTTCnCATCAGGTGGCCAAAGTATTGGAGCTTGAGCTTCAGTATCAGTCCTTCCAATGAATATT | T | G |
| 680 | GAATATATAGAATATTTTGCATACATTCATACTAAACAGATAAAGTACAAGAGGTGTTTnGTGAGTTGCAGAGTTTCGTGCGATACAGAGGGAAGACCCCATTGAGAAAGCATTGGATC | A | G |
| 681 | TGCATACATTCATACTAAACAGATAAAGTACAAGAGGTGTTTGGTGAGTTGCAGAGTTTnGTGCGATACAGAGGGAAGACCCCATTGAGAAAGCATTGGATCCTTAAGACCTGAGGGCC | C | T |
| 682 | CGATAGGGAAAGTATCTTCTGGGATAAGAGCTCGGTGACCAGCTGAGAACAGGGGGGCCnTCCCATACTGAGAACTGCGTGAGCAAAGGCCCTGAGGCAAGAGGGCGCATGGGTCTCAG | G | A |
| 683 | TAACAGACCTTGAAGTGCCACTGGCTTCACGCCACAAAGGTTTCCTTGTAGGTGATGTCnACCCACTGGCAAGTGGGGGGTATGGGATTCTGGTCCACACACTGATTCAGCGACCCAGG | AGAGCC | A |
| | | T | |
| 684 | CTCTTTCCCATTGCTCCTTTGGCACTGCCCACTTCCTCCTGGCCACCCTCTGGCTTCACnGATCCTCACAGTCCACCCACCGCCACCCTGCCCCCGGCGGCCTCCCGCTTCAAGCCCTG | C | T |
| 685 | GCACTGCCCACTTCCTCCTGGCCACCCTCTGGCTTCACTGATCCTCACAGTCCACCCACnGCCACCCTGCCCCCGGCGGCCTCCCGCTTCAAGCCCTGACAGTGTCCCGGCCCTCCCCC | T | C |
| 686 | AACACTATCCTCGCCCACCCGCCCCCCTGGGTGCCAGAGACTTGCTGACTCCTGCCAAAnTCGGCATGGTCACCCCCTGCCTCAGGGCCAAACTACGGCTCCAGCAGCACTGACTGGTC | T | G |
| 687 | CTGGGTGCCAGAGACTTGCTGACTCCTGCCAAAGTCGGCATGGTCACCCCCTGCCTCAGnGCCAAACTACGGCTCCAGCAGCACTGACTGGTCCAGCCACTGTCACCCCTTGGGTCCTC | A | G |
| 688 | ACAGCTTCACTGTAGCAGCAGCAGCAGCAAAGACTGTCAGCACCCCTCACGGGCCCCAGnGCAGCAGCATCCAGACCACAGGAAGTCCCCAGGACCACTGGCTGACGATGGGCCTTCAG | C | G |
| 689 | CTCTTAACAGGAAACTGAAAGCAGACCCTGTGCCTGTGGGCTCAAACGCTGGTGGGAGCnCGCCTACCCCGGGTCAGCCAGGTTTCTGCCCCCAAGTCTGTCACTGAATCTTTCTCCGC | T | C |
| 690 | GCAGACCCTGTGCCTGTGGGCTCAAACGCTGGTGGGAGCCCGCCTACCCCGGGTCAGCCnGGTTTCTGCCCCCAAGTCTGTCACTGAATCTTTCTCCGCATTTCCAGCCTGGATGGGAA | G | A |
| 691 | TCTGCCCCCAAGTCTGTCACTGAATCTTTCTCCGCATTTCCAGCCTGGATGGGAAGGGCnTTTGGGGGAGAACGGATCCATGTATGTAGATGGCTGAGTCCCTTCCCTGCTCACCTGAA | A | G |
| 692 | TGAAACTATCACAACATTGTTAACTGGCTACACCCCAACACAAAACAAAAAATTCGAAAnAAAAACTTCCTCCACTTTTCATACTGTTAACCTTTCCTGTTTTCAACTTTGTTTTTCCT | GAAAAA | G |
| | | A | |
| 693 | CTCTTTTTTTCAAAATGCTTCAGTTATATCAGGCACATAAAAATGTATAGATAATCTCAnAGCAAACTTCAATGCTCACTACCTAGGTTGGTTTGGGGGGCTGCTTTGCTTTTTCCCTC | A | C |
| 694 | TTATATCAGGCACATAAAAATGTATAGATAATCTCACAGCAAACTTCAATGCTCACTACnTAGGTTGGTTTGGGGGGCTGCTTTGCTTTTTCCCTCCCTCCTCCTCCTACACATTCACT | T | C |
| 695 | GCTTTTTCCCTCCCTCCTCCTCCTACACATTCACTACATGTGTATGTCCATTTATAATAnAGATATATGTTTTTCATGCTTTTAAATTCTATGTAGATGGACCCATAGTCTAGGTATCC | T | C |
| 696 | CTTTTTCCCTCCCTCCTCCTCCTACACATTCACTACATGTGTATGTCCATTTATAATACnGATATATGTTTTTCATGCTTTTAAATTCTATGTAGATGGACCCATAGTCTAGGTATCCT | G | A |
| 697 | ATGTTTTTCATGCTTTTAAATTCTATGTAGATGGACCCATAGTCTAGGTATCCTGATATnACCTGGTTTTTTTTAGCCCAACACTTTATGAGTTTTATCCACGTTGATATAGACACTCT | C | A |
| 698 | ATATTCAAATCATGTAGGGTACTGAATAAATAATGGAAACCTCCCTTTACCTACTTCTCnATCTCAAAATTTACCACCATGACCACCTATGTGTATTCTGCCAGCCCTTTTTCTAGGCA | C | T |
| 699 | TAACTCCAGTGTCTGCTGGATAAAAGCCACACTCCCCATTCTGGCACTCAGGGCCTCGTnTCTCAGTTCCCCAGTCCTCAGGCAGCCTGTACAGGGTCTTACTGAACCAGCGTTCCTCT | C | G |
| 700 | AAGCCACACTCCCCATTCTGGCACTCAGGGCCTCGTGTCTCAGTTCCCCAGTCCTCAGGnAGCCTGTACAGGGTCTTACTGAACCAGCGTTCCTCTGAGAGCCCACGTGTTCCAGACCT | G | C |
| 701 | CTGGCACTCAGGGCCTCGTGTCTCAGTTCCCCAGTCCTCAGGCAGCCTGTACAGGGTCTnACTGAACCAGCGTTCCTCTGAGAGCCCACGTGTTCCAGACCTCGGTCAGAGAGAGCAGG | C | T |
| 702 | GCCTCGTGTCTCAGTTCCCCAGTCCTCAGGCAGCCTGTACAGGGTCTTACTGAACCAGCnTTCCTCTGAGAGCCCACGTGTTCCAGACCTCGGTCAGAGAGAGCAGGACAGGGGTCAAT | A | G |
| 703 | GTGTTCCAGACCTCGGTCAGAGAGAGCAGGACAGGGGTCAATCCTGAGTCTCATGCCCCnCCCGCACTAGCCAAGTTAGCCCGCTCATGGGTGCCACTTCCACTTCTGAGAGGCTGGCT | A | G |
| 704 | GAGCAGGACAGGGGTCAATCCTGAGTCTCATGCCCCGCCCGCACTAGCCAAGTTAGCCCnCTCATGGGTGCCACTTCCACTTCTGAGAGGCTGGCTCCAGTGTCCTCCCTGACTTGAGA | A | G |
| 705 | CTGCAGACAGTGGGGTCTGGAGGCCTCACTTGGTAGACAAAGGATGGTGCATTATAATTnTGTTTTCACTTAGCAGTTCACTTCTCTGCCACACTCGCTAACTAAGATCCAGGAAGAAA | C | A |
| 706 | ATGCCAATTGGATTCATGAGCTTCGCCTCTCCCACTAATGGGGTCAGGCTCTGGCGGCCnTTATTGCACAGAGGCTCCGGGAGAAGCAAACACACAGGCGAGACTTCACCGCCACTTAT | T | A |
| 707 | AGGGCTGCGCAGAACCCACATTCCCTAATGTGGGGGATCGACATACCTCCCTCAGCATCnAGGCCTGAAGCTTCTGAACCCTGACTTTAAGGCCGGGCACACTGAAACTTGTCGGAGAG | T | C |
| 708 | TCGACATACCTCCCTCAGCATCCAGGCCTGAAGCTTCTGAACCCTGACTTTAAGGCCGGnCACACTGAAACTTGTCGGAGAGACCTAGCCACAGGGAGTTCCTAGTCTGAGAGAAACCC | T | G |
| 709 | GAAGTAAGTATATTATTATTCCTATTTTATGGTGAAGGTCAGAAAGGTTAGATGTCTGAnTTTACACACTGTTAAGTGATGGGGTCAGAATTCAAACCCGGAGTCATCTTAGACACTAC | A | G |
| 710 | GTATATTATTATTCCTATTTTATGGTGAAGGTCAGAAAGGTTAGATGTCTGAGTTTACAnACTGTTAAGTGATGGGGTCAGAATTCAAACCCGGAGTCATCTTAGACACTACACTTTGC | T | C |
| 711 | AGTGATGGGGTCAGAATTCAAACCCGGAGTCATCTTAGACACTACACTTTGCTTCTACTnTTTTTTTTTCCCCTAATAAAAGTCTTTTTTCTTCAATTAATTTGCTTCTCTTGACTTCT | CT | C |
| 712 | GAAGTCCCAGGGCCATAGGCCTGGGCCAAGAAGCACCTTTGATGGTGGGTGAGGGGTCTnATGAAAAGGGCAAGAAAGGAACTGTTTTCAATCATTCTCAGGACGCAAGAATTTCTGGT | G | C |
| 713 | CAAGAAGCACCTTTGATGGTGGGTGAGGGGTCTCATGAAAAGGGCAAGAAAGGAACTGTnTTCAATCATTCTCAGGACGCAAGAATTTCTGGTACTGGGCCACCTATACCTTCAGGCCC | C | T |
| 714 | GTGGGTGAGGGGTCTCATGAAAAGGGCAAGAAAGGAACTGTTTTCAATCATTCTCAGGAnGCAAGAATTTCTGGTACTGGGCCACCTATACCTTCAGGCCCTCTCAGAAATCCTTGGGA | T | C |
| 715 | CCACCTATACCTTCAGGCCCTCTCAGAAATCCTTGGGATATTATTGACAGGCAACAAACnCCGCCAAATTGTCTGCCATGCCTACACAATGTGTGGGCACAGAGGGTTATGGCTTCATT | G | A |
| 716 | AATTGTCTGCCATGCCTACACAATGTGTGGGCACAGAGGGTTATGGCTTCATTTGCTCCnTCTATAATCCATGTGTAGAGACCCTCTTTTCCGAACCCAAAGTGTTCTGTCTAGGTCAA | G | A |
| 717 | CAATGGATGAATGCATTAATGTAATCTCAAAGATTCGCAGTGAAACTAAGCCATTGCCGnTGAAAGGGTCCAAGCCCAGGGTGCAAAGATTATTGCCCCAATTCCACACTCCGGGCTAC | C | G |
| 718 | TAATGTAATCTCAAAGATTCGCAGTGAAACTAAGCCATTGCCGGTGAAAGGGTCCAAGCnCAGGGTGCAAAGATTATTGCCCCAATTCCACACTCCGGGCTACTTGTACCAGGAACCAC | A | C |
| 719 | CGGGCTACTTGTACCAGGAACCACCCTGCCCCCTCCACTGGAACGACCACGCTGCGCATnCCAGGGAGGCTCGCCTCCTCTGGCGGCCCAGCTTCCCTTTCTCCTCGCGTCTTTCTGTC | C | T |
| 720 | TCGGGATTTTCTCTCCCTTCTTCCTGCCGCCCTCTCCTCTACCCTGTGACCCCTGCCCCnTCCCCGCTGTGCCCCCACCACACTCTGTCAAACTGGGGCCAGGAGACCAGCGCTAAGGT | C | G |
| 721 | AAGGTCACTGGGTTTCAACCTGCAGAATGGGAAGCCATCTCCCCTGTCTACAGGTGAATnCTGTTACTCTGCCTCCAGCCATGGACTGCTTGCTCTTGTGGGAAAGTGGAAAGCAGCTG | T | G |
| 722 | CAGGTGAATGCTGTTACTCTGCCTCCAGCCATGGACTGCTTGCTCTTGTGGGAAAGTGGnAAGCAGCTGACCAGGGACCGGGGATGCTAGTCAACCAGTAATTCTCAGTGTAACCTAGA | G | A |
| 723 | AGTCCCTAAACTATCAGGTTGGGATTCACCAAGGAGATTCCTGAAAAGGAGAGAAAGTAnCGTGGCCTTGCCTAAACCTCCACATTTGCCCTTATAACATCTCTGCCTTCACTGGCCTG | G | A |
| 724 | CTAAACTATCAGGTTGGGATTCACCAAGGAGATTCCTGAAAAGGAGAGAAAGTAACGTGnCCTTGCCTAAACCTCCACATTTGCCCTTATAACATCTCTGCCTTCACTGGCCTGGGCGT | T | G |
| 725 | AAAAGGAGAGAAAGTAACGTGGCCTTGCCTAAACCTCCACATTTGCCCTTATAACATCTnTGCCTTCACTGGCCTGGGCGTGGAGTGATTGCATGTTTCTTAAGGACCCACTGTGTGCC | C | CT |
| 726 | TCTGCCTTCACTGGCCTGGGCGTGGAGTGATTGCATGTTTCTTAAGGACCCACTGTGTGnCAAGCACGCATATATCACAGCCCCACTCTCAGAGCGCTCGTGGCATAATAGAGCAGCGT | T | C |
| 727 | CACTGGCCTGGGCGTGGAGTGATTGCATGTTTCTTAAGGACCCACTGTGTGCCAAGCACnCATATATCACAGCCCCACTCTCAGAGCGCTCGTGGCATAATAGAGCAGCGTGGCAGGGC | A | G |
| 728 | GGGGGTGAAGCTTCATGCCAGGCTGTACTGCACAATGAGAGAGCGAGGGCCAGGTTCTGnCTGCACTCCCCCGGTCAGCAGCCTCCTCAGCGCAGGACACGACCTGCTCAGCCTCTGAC | T | C |
| 729 | TATCCTGTATGGGAACAGGTGGGGAAGCTGTCTGCAGGCAAGAAATGGGCAGACTGAGGnCATGGAGCACAGCCTTCTCTCCCCTCCACCTCTTGCTGGCACAAGCAAACTGAATGCAG | G | A |
| 730 | CTATAATAGTTAATGGGTATTAAATATTTACCATCATGCACTGTGCTAAGTGCCCCACAnGCATTATCTAATTTCATCCTCAAACAACTTTGGAAATACGTGCTATTAATAATCTCATT | T | C |
| 731 | ACTGTGCTAAGTGCCCCACACGCATTATCTAATTTCATCCTCAAACAACTTTGGAAATAnGTGCTATTAATAATCTCATTTTACTGATGAGGATTCTACGTAACATGCCTAAGGTCACA | T | C |
| 732 | CTCAAACAACTTTGGAAATACGTGCTATTAATAATCTCATTTTACTGATGAGGATTCTAnGTAACATGCCTAAGGTCACACAGCTATTTAGTGAGGGCCCTGGGGATGGAATTCACAGC | A | C |
| 733 | GTTCTATATCTGTTGGACTAAGTGGAGAAGGGCTCTGAATTCCATGCTAGGGAGCTTGAnCTTTACACCATGGGCAATGGGGAGACACTGGACGGTTGTAAGCAGGGAGAGACGGGTCA | C | A |
| 734 | GAGCAAGAGGCCATTTTTCTGGCAAATCCCAATTCCTCCTTGTATTTTTTTTAAGTCTTnTCTTATCTCTTAGGGCAAGACTTATACGCACAGTACATCATGCAAAATGCTGGACTGGA | A | G |
| 735 | AGCAAGAGGCCATTTTTCTGGCAAATCCCAATTCCTCCTTGTATTTTTTTTAAGTCTTGnCTTATCTCTTAGGGCAAGACTTATACGCACAGTACATCATGCAAAATGCTGGACTGGAT | C | T |
| 736 | GGATGAAGCATAAGCTGGAACCAAGATTGCAGGGAGAAATATCAGTAACCTCAGATATGnGGATAAGTAACCTCAGATATGCGGATAACACCACCCTTATGGCAGAAAGCAAAGAGGCA | T | C |
| 737 | AGAGCCTCTTGATGAAGGTGAAAGAGGAGAATGAAAAAGCTGGCTCAAAACTCAACATTnGGAAAACAAAGACCATGGCATCGGGTCCCATCACTTCACGGCAAATAGATAGGGAAACA | C | T |
| 738 | GAAGGTGAAAGAGGAGAATGAAAAAGCTGGCTCAAAACTCAACATTTGGAAAACAAAGAnCATGGCATCGGGTCCCATCACTTCACGGCAAATAGATAGGGAAACAATGGAAACAGTGA | T | C |
| 739 | GAGGAGAATGAAAAAGCTGGCTCAAAACTCAACATTTGGAAAACAAAGACCATGGCATCnGGTCCCATCACTTCACGGCAAATAGATAGGGAAACAATGGAAACAGTGACAGACTTTAT | A | G |
| 740 | ACTCAACATTTGGAAAACAAAGACCATGGCATCGGGTCCCATCACTTCACGGCAAATAGnTAGGGAAACAATGGAAACAGTGACAGACTTTATTTTCTTGGGTTCCAAAATCATTGCAG | G | A |
| 741 | AAGAAAAGCTATGACAAACTTAGCCAGAGGCATTATTTTGCTGACAAAGGTCTGTCTAGnCAAAGCTATGGTTTTACCAGTAATCATATATGGATGTGAGAGTTGGACTATAAAGAAAG | T | A |
| 742 | GACAAAGCTATGGTTTTACCAGTAATCATATATGGATGTGAGAGTTGGACTATAAAGAAnGCTGAGTGCCAAAAAATTGATCCTTTTGAACTGTGGTATTGGAGAAGACTCTTAAGAGT | G | A |
| 743 | GGTTTTACCAGTAATCATATATGGATGTGAGAGTTGGACTATAAAGAAAGCTGAGTGCCnAAAAATTGATCCTTTTGAACTGTGGTATTGGAGAAGACTCTTAAGAGTCCCTTGGACAC | G | A |
| 744 | TTTACCAGTAATCATATATGGATGTGAGAGTTGGACTATAAAGAAAGCTGAGTGCCAAAnAATTGATCCTTTTGAACTGTGGTATTGGAGAAGACTCTTAAGAGTCCCTTGGACACCAA | G | A |
| 745 | GAAGCTCCAATACTTTGACCACTTGATGTGAAGAACTGACTCACTGGAAAAGACCCTGAnGCTGGCTGGGAAAGATTGAAAGCAGGAGGAGAAGGGGATGACAGAGGATGAAATGGTTG | T | TGCTG |
| 746 | TCACTGACTTGATGGACATGAGTTTGAGCAAGCTCTGGGAGTTGGTGATGGATAGGGAAnCCTAGCGTGCTGCAGTCATGGAGTCTCAAAGAGTCAGACAGGACTGAGCAACTGAACTA | A | G |
| 747 | CCCTTCTCTGGGCAGCTCATGATATCACTGGCTTTTCCAGCAAGTGGGGTGGGGAGTTAnAGGAAATGGCCTGGGGCATCAGATAAGCTGAAGAAAGAGGCCGAGTAGCTGGAAAGACG | G | C |
| 748 | GGGTCCCAAGATGCTGCTCTGCGTCACCTTGCTTCTCCTCCTGGGGCTGTCTGCATGCAnTGTGGCAGGTGACAAGGAACTGGCAATCAATGCTGAAGTTGGCTCCTGGGTGGCTGTGA | T | C |
| 749 | CCTTGCTTCTCCTCCTGGGGCTGTCTGCATGCACTGTGGCAGGTGACAAGGAACTGGCAnTCAATGCTGAAGTTGGCTCCTGGGTGGCTGTGACCCTGGAGGTAGGTACCTTTGGGGAA | G | A |
| 750 | AGGTAGGTACCTTTGGGGAAAGCAGGGCATGATAGCAAGAGAGCAGGCTGTACACTGCAnGGTAGGGTGTCTGCCTCCCCTCTCTGGGCATGTTCAAGGCTGTTGCCCCTGGCCTGATA | C | T |
| 751 | TCAGGCTTCCCTGTCCTTCACCATCTCCCAGAGCTTGCTCAAACTCATGTTCATCAAGTnGGTGATGCCATCCAACCATCTCATCCTCTGTCCCCTTCTCCTCCTGCCTTCAATCTTTC | C | T |
| 752 | GAGCTTGCTCAAACTCATGTTCATCAAGTTGGTGATGCCATCCAACCATCTCATCCTCTnCCTTCTCCTCCTGCCTTCAATCTTTCCCAGCATCAGGGTCTTTTCCAGTGAGTCAGTTC | GTCA | G |
| 753 | CTCATCCTCTGTCCCCTTCTCCTCCTGCCTTCAATCTTTCCCAGCATCAGGGTCTTTTCnAGTGAGTCAGTTCTTCACATCAGGTAATCAAAGTATTGGAGCTTCAGCTTCAGCATCAG | T | C |
| 754 | CCTTCAATCTTTCCCAGCATCAGGGTCTTTTCCAGTGAGTCAGTTCTTCACATCAGGTAnTCAAAGTATTGGAGCTTCAGCTTCAGCATCAGTCCTTCCAATGAATATTCAGGACTGAT | G | A |
| 755 | ATATTCAGGACTGATTTCCTTTAGGATTGACTGATTTGGTCTCCTTGCTGTCCAAGGGAnTCTCAAGAGTCTTCTTCAATACCACAGTTCAAAAGGATCAATTTTTTGGCACTCAGCCT | G | C |
| 756 | TCCTTTAGGATTGACTGATTTGGTCTCCTTGCTGTCCAAGGGACTCTCAAGAGTCTTCTnCAATACCACAGTTCAAAAGGATCAATTTTTTGGCACTCAGCCTTCTTTATAGTCCAACT | C | T |
| 757 | CCAAGGGACTCTCAAGAGTCTTCTTCAATACCACAGTTCAAAAGGATCAATTTTTTGGCnCTCAGCCTTCTTTATAGTCCAACTCTCACATCCATACATGACTACTGGAAAAACCATAG | G | A |
| 758 | GTCCAACTCTCACATCCATACATGACTACTGGAAAAACCATAGCTTTGACTAGACAGAAnTTTGTCAGCAAAATAATGTCTCTGCTTCTTAATATGCTGAGTTTGTCATAGCTTTTCTT | C | T |
| 759 | AGACAGAATTTTGTCAGCAAAATAATGTCTCTGCTTCTTAATATGCTGAGTTTGTCATAnCTTTTCTTCCAAGGAGCAAGCATCTTTTAATTTCATGGCTGCGGTCACCATCTGCAGTG | C | G |
| 760 | TGTCACTGTTATCTATTTGCCATGAAGTGATGGGACCCAATGCCATGAACTTTGTTTTCnAATGTTGAGTTTTGAGCCAGCTTTTTCATTCTCCTCTTTCACCTTCATCAAGAGGCTCT | G | A |
| 761 | ATACAGGTTTCTCAGGAGGCAGATAAGGTGGCCTGGTATTCCCATCTCTTTAAGAATTTnCCACAGTTTGTTGTGATCCACACAGTCAAAGGTTTTAGCGTAGTCAACGAAGCAGAAGT | T | C |
| 762 | AAATCCAGCTTAAACATCTGGAAGTTCTCGGTTCACATACTGTTGAAGCCTAACTTGGAnAATTTTGAGCATTACTTTGCTAGCATGTGACATTAGTGTAGTTGTGCGGTAATTTGAAC | G | A |
| 763 | TTAGTGTAGTTGTGCGGTAATTTGAACATTCTTTGGAATTGCCTTTCTTGGGATTGGAAnGAAAACTTTTTCCAGTCCTATGGCAACTGCTGAGTTTTTCCAAATTTTCTGGCATATTG | T | C |
| 764 | TGGAACGAAAACTTTTTCCAGTCCTATGGCAACTGCTGAGTTTTTCCAAATTTTCTGGCnTATTGAGTGCAGCACTTTCACAGCATCATCTTTTAGGATTTGAAATATCTCAACTGGAA | G | A |
| 765 | AGATGTCTAGTCTTTCCCATTCTATTGTTTTCCTCTATTTGCATTGATCACTTAAGAAGnCTTTCTTATCTCTCCTTGCTATTCTCTGGTCTCTGCATTCAGATGGATATATCTTTCCT | G | C |
| 766 | TTTTCTTGGGGATGGTTTTGATCACCGCCTCCTATACAATGTGAACCTCCGTCCATAGTnCTTCAGGCACTCTATCAGATCTAATCCTTTGAATCTATTTGTCACGTCACCTGTATAAT | C | T |
| 767 | AATTGTAAGGGATTTGATTTATCTCATACCTGAATGGCCTAGTGATATATGGAATTAACnGTATGTGTGTTCATGCTTCATTTAACAAACTTATTTAATATATACATGTTCCTGACACT | A | AGTAT |
| 768 | CAATGTCTTATTCTGTGTATCTGTGCCTCGGAGGACATTGTCCAGCACATCCTTTCAAGnGGGTCAACTTGGAGTGGGATCACTGTTTTGTCTCATGTGTAGAAACTTATTCCCTGACA | C | G |
| 769 | AATCTCACAGCTAGAGACACAGCGTCTGTGCCCCACCCCATCTAGTCAGAGAGACAAAGnTCCTGGCCTCGGAGGTTCCCATCAGACAGGGGCACGAGGCCAGTGGACTAGAGACACAC | G | A |
| 770 | AGCAGGCTGAGGAGATAAAGGTTCAAGTCTCAGCTTCTCCACCGACTCTGTGTGACCTGnGGACATCAGTTTCCTCACTTGTAAAATGGGAGAGGGGGGGAAAAAGATGGCCCCCAAGG | A | G |
| 771 | CCGACTCTGTGTGACCTGGGGACATCAGTTTCCTCACTTGTAAAATGGGAGAGGGGGGGnAAAAGATGGCCCCCAAGGTTTTTTTTCCAGCTCTGACTTGGAGTCTGTGCCCATCAGGG | G | A |
| 772 | CGACTCTGTGTGACCTGGGGACATCAGTTTCCTCACTTGTAAAATGGGAGAGGGGGGGAnAAAGATGGCCCCCAAGGTTTTTTTTCCAGCTCTGACTTGGAGTCTGTGCCCATCAGGGG | G | A |
| 773 | GTAATAGGGACCACTCTACACGGTTACGTGGGAAACATCATCGATTTCAACCAGTGCCTnGTGGGGCTTTCCATGCTGCAGCTGCATCTGAAGCTTCTTCTTGGCTCACTCTCATTTAA | C | T |
| 774 | CCAGTGCCTTGTGGGGCTTTCCATGCTGCAGCTGCATCTGAAGCTTCTTCTTGGCTCACnCTCATTTAACATCCCTCTACTTCCCAAGACACAGAATTTGGGATATAGGAACCATCTCA | C | T |
| 775 | CCATCTCATCTGAATGAGGAATGATGCCTCTGTTAGGGATGATCTGATTTTCTCCCTTGnTCTCAGGACTCCCAGTTAGCCCTGGAGCTGGGGATTTGAGTTAAAGGGGTAAGAAGAGC | A | C |
| 776 | GATCTGATTTTCTCCCTTGCTCTCAGGACTCCCAGTTAGCCCTGGAGCTGGGGATTTGAnTTAAAGGGGTAAGAAGAGCTTCTGGTCAGGGCAAGAGTCCTTCCTGAGTAAAAGACACC | A | G |
| 777 | AGCCCTGGAGCTGGGGATTTGAGTTAAAGGGGTAAGAAGAGCTTCTGGTCAGGGCAAGAnTCCTTCCTGAGTAAAAGACACCTCCCAACGGGCCCCAGAAACCTAATGGGTGGGAGAAA | A | G |
| 778 | GAAGATCCTCTGGGGGAAGATCCCCTGGAGAAGGAAACGGCAACCCACTCCAGTACTCTnGCCTGGAAAATCCCATAGATGGAGGAGCCTAGGAGGCTGCAGTCCATGGGGTCACAAAG | T | C |
| 779 | GGAAAATCCCATAGATGGAGGAGCCTAGGAGGCTGCAGTCCATGGGGTCACAAAGAGTCnGACACGACTGAGTGACTTCACTTTGAGCTAAAGGATGAAAATGTCTTTGCCTGAGGGAG | A | G |
| 780 | CTAATCACAAACCTGTGTCCCCATCTGTAGATGGATATTTCACATGGTGGTTGTGAAGCnTAAATGAGATTATGCCTGGAGTGTCTGAGCCTTTGAGTTAGATGACCTGGGTTCGAGGC | A | G |
| 781 | TTCAGACTGTTTTGTTGTCAGCCTGCCCTCAACTCTAAGGAACTCTCTGCTTCTTCAGGnAGATGTCATCCCCAGCATCCTGCTTCAACTCCGGAACGTGAAGAAGGGGAAAGCCAGCC | A | T |
| 782 | TAAGGAACTCTCTGCTTCTTCAGGTAGATGTCATCCCCAGCATCCTGCTTCAACTCCGGnACGTGAAGAAGGGGAAAGCCAGCCAGTTCTTTGGGCTAATGGGGAAGCAGGTAGGAGGT | G | A |
| 783 | CAGGTAGATGTCATCCCCAGCATCCTGCTTCAACTCCGGAACGTGAAGAAGGGGAAAGCnAGCCAGTTCTTTGGGCTAATGGGGAAGCAGGTAGGAGGTGAGTGACAGATGTGGGCGGA | A | C |
| 784 | TTGGGCTAATGGGGAAGCAGGTAGGAGGTGAGTGACAGATGTGGGCGGACACCCAGGGAnAAAGGCTTATGGGTGGGTTCTGTTACCCCAAACAGAGGCCCTAACTTAAAGCCCAGGAC | C | T |
| 785 | TGGGGAAGCAGGTAGGAGGTGAGTGACAGATGTGGGCGGACACCCAGGGATAAAGGCTTnTGGGTGGGTTCTGTTACCCCAAACAGAGGCCCTAACTTAAAGCCCAGGACTCAGCATCC | C | A |
| 786 | TACCCCAAACAGAGGCCCTAACTTAAAGCCCAGGACTCAGCATCCCTTACTCCAGTCCAnTAGTGGTCACTCACGCTATGGAATGGGATACAGTGTCCATATCAATTGTTATCCAAATC | A | G |
| 787 | AAACAGAGGCCCTAACTTAAAGCCCAGGACTCAGCATCCCTTACTCCAGTCCAGTAGTGnTCACTCACGCTATGGAATGGGATACAGTGTCCATATCAATTGTTATCCAAATCAGGACA | A | G |
| 788 | CAGCATCCCTTACTCCAGTCCAGTAGTGGTCACTCACGCTATGGAATGGGATACAGTGTnCATATCAATTGTTATCCAAATCAGGACATTTCAGAGAGTGTTAATTATGCCAGGCAACA | T | C |
| 789 | CACTGTCCATTTGGAGGACATTTTGGCAATATCCATCAAAGTTGGCAACATGCATACCTnCTGACTGATAGGTTCGCTTCAGGAAATCCATCAAATAGAAATGCTATTGCTACATAAGC | T | TCTGA |
| 790 | AAGGTGAATTCTTAACCACTGGACCACCAGGGAAGTCTGGCACATCCATAGCCTTTGTGnCCATTGAAGTGGATGAGGCAGATCTGTGGAAAGATAAAGATTTCCAAGATATTGGCTAA | G | A |
| 791 | CACACATGAAAACATAGGGAGAGTTCCAGGATAAATATCAAACTGTCACCAGTGTTTTAnATCTAGGATAGAAGTGAAGGTGAGGGAGAGCAAAAGTTAAGGGGGACTTTTACTACACT | T | C |
| 792 | GAGGGAGAGCAAAAGTTAAGGGGGACTTTTACTACACTAAACTCTACCTTACCACTTGAnAGTGGGCATTTATTCAGAAACTGCTTTTGTAACTTTAAAAAACACATTTAAAAATAGAG | C | T |
| 793 | CTTTAAAAAACACATTTAAAAATAGAGAAAATCTTCCTGCTACATGCACCATAAAATAAnATAGAGAAAATTAACTTGTGACCAAACTTTGGCCTCAGTTTCTTCCACAAAATGAGTTT | A | AAT |
| 794 | GCACCATAAAATAAAATAGAGAAAATTAACTTGTGACCAAACTTTGGCCTCAGTTTCTTnCACAAAATGAGTTTTGGCCTGGGCCATTTCAGAGATCTCTTTCAAATCCAGAAACCCAG | C | CCACAA |
| | | | AATGAG |
| | | | TTTTGG |
| 795 | CCTTTATCACCAGCAGGGAAACTAAGGTTCACATAGGCTAAGTACTTTCCCAAGGACGCnTGACTAGCACCAGGCAGACTCCACAGTATGCACACTTCCCACTACCCTAAACTGCCTCC | T | C |
| 796 | GGCAGACTCCACAGTATGCACACTTCCCACTACCCTAAACTGCCTCCTACCCAGATGTAnCTGGTGTGGCCCAGTCACACCAGGACAACAGGGGGAGACCAGAGTCCCCAAAGACCCAC | C | T |
| 797 | CCACAGTATGCACACTTCCCACTACCCTAAACTGCCTCCTACCCAGATGTATCTGGTGTnGCCCAGTCACACCAGGACAACAGGGGGAGACCAGAGTCCCCAAAGACCCACCAAAGGCA | A | G |
| 798 | CTAAACTGCCTCCTACCCAGATGTATCTGGTGTGGCCCAGTCACACCAGGACAACAGGGnGAGACCAGAGTCCCCAAAGACCCACCAAAGGCATGGATCCCAAAGAGCCATGAGCTGTT | A | G |
| 799 | AAACTGCCTCCTACCCAGATGTATCTGGTGTGGCCCAGTCACACCAGGACAACAGGGGGnGACCAGAGTCCCCAAAGACCCACCAAAGGCATGGATCCCAAAGAGCCATGAGCTGTTTC | T | A |
| 800 | ATTTCTTCTCTTTCCCCTAGGAATACCTCCCATCCAGTCAGAGGGAACAGGTAAGTGTTnTCCACACCTTCCCCCCACATGCTCCCTGGGGAAGACCGAGTTGTCAGGGAGGTGAGGGA | G | A |
| 801 | GCATAGGCTTCCTGGTTGGTTCCTTGGAGAGGTGGTCTGTGTGCGCCGAGGGAGCAGCTnCAAGGATGAGGAGATGGCGATGAATCCAGTTCTGCCAAAGGTTCAAAATTACCATGGGT | C | G |
| 802 | CAATCCAGAAGGACCTTAAAACTGAAAACCCTAACCCTGTCTTCATGGCCTGGGCATGGnGCCCTCCTTGGCATACTGCCCTGCACTGTTCTGCTGGGTCACATGGCTGGTGTCCTGAG | C | T |
| 803 | AAACTGAAAACCCTAACCCTGTCTTCATGGCCTGGGCATGGTGCCCTCCTTGGCATACTnCCCTGCACTGTTCTGCTGGGTCACATGGCTGGTGTCCTGAGAGTAAAAACCACCCCACG | A | G |
| 804 | ACCTCAGTTCCTAGAGAAATTACTCTATTCCTGAGTCCCCAGTTCTGGTCCTGTCTTTTnTGGTTCCCAAGACAGTCTTCCTCTGCTTCACTTTCTCTTTTTCTTCTCTGTCTCCTCTC | T | TTGGTT |
| | | | CCCAAG |
| | | | ACAG |
| 805 | CAGTTCTGGTCCTGTCTTTTTTGGTTCCCAAGACAGTCTTCCTCTGCTTCACTTTCTCTnTTTCTTCTCTGTCTCCTCTCTTCATCACAAGGGTATCAGCGAGAACCAGTGGTCCAGGG | C | T |
| 806 | GGTATCAGCGAGAACCAGTGGTCCAGGGGCGAGGAGGAGCAACTACAGAAGGTAGGTGAnAGCTTCTCCAGCAGCACACACAGGCACATTCTGCTCCCCACTCTCAGGGCCAGCTGAAG | C | T |
| 807 | GAACCAGTGGTCCAGGGGCGAGGAGGAGCAACTACAGAAGGTAGGTGATAGCTTCTCCAnCAGCACACACAGGCACATTCTGCTCCCCACTCTCAGGGCCAGCTGAAGCCACCGAGAAT | C | G |
| 808 | AGGTAGGTGATAGCTTCTCCAGCAGCACACACAGGCACATTCTGCTCCCCACTCTCAGGnCCAGCTGAAGCCACCGAGAATGCTGTGTGGTTCTCCAGACCCTGGAACACGCCCCTTAT | A | G |
| 809 | CTTCTCCAGCAGCACACACAGGCACATTCTGCTCCCCACTCTCAGGGCCAGCTGAAGCCnCCGAGAATGCTGTGTGGTTCTCCAGACCCTGGAACACGCCCCTTATTCCCTAATTGTTA | A | ACC |
| 810 | TCTTAGGTATTTTCTGTCTTCAACAACCTAAATAAAGGCAATATCACTCTGGGAGGGGTnATCAGCTGACGCTTTCTGGCCTGGGCCACAACCACAAAGGCGTTCAAGGGGATGTTCAA | T | C |
| 811 | TTGAGCCATTTCATTTGAAGGGCCATGGAAGTCACCCCAGTGCCAACATGGAAATGCACnTCCCTCCATTCAAAGCGCTGGCAGGAACCTTAGAAATCATCATTTTTCAGGGATGGAAA | G | A |
| 812 | TCATTTGAAGGGCCATGGAAGTCACCCCAGTGCCAACATGGAAATGCACATCCCTCCATnCAAAGCGCTGGCAGGAACCTTAGAAATCATCATTTTTCAGGGATGGAAACAATTTGCCC | G | T |
| 813 | GTCACACGGTGAACTGTTAACAGAGCCAGGACTAAACACCGTCTCCTGCTCCCTAGTCCnGGCACTTCATGGGACCCTCAGAGGGCCATTGGAATTTTAAGTGCTGGATTACCACTCCC | G | A |
| 814 | AGAGAGGATTAGTGAGGGTTGGCTCATCCACTATCTAGAGGCATTTATGATTGAAAAACnGAAGCTCTGCCAGATTAACCTGGAATTTGATTGCCCTAAATAACTACAGTTGGAGAAAC | A | G |
| 815 | GGCTCCCCTAACCACTGCTTATGTGCAGCACTCTTCCCAGGCAAAGAGGCTGAGAACCAnGGGTCAGAGTGAGAGCCCCCAGCCCCGCCTCCCAGAGGACGAAACCCCTCTCCTTACCT | C | T |
| 816 | AGGACGAAACCCCTCTCCTTACCTGGACGTGGCAGCCAACCAGCCGACCAGGCCAGCCCnCTGCTCCAGGTCTTCTGTGCTCGTGTGTCCCTCCCACCACGCTGAACTCACTGGGCTCC | T | G |
| 817 | AACCCCTCTCCTTACCTGGACGTGGCAGCCAACCAGCCGACCAGGCCAGCCCGCTGCTCnAGGTCTTCTGTGCTCGTGTGTCCCTCCCACCACGCTGAACTCACTGGGCTCCTCCCAGG | T | C |
| 818 | ACCCCTCTCCTTACCTGGACGTGGCAGCCAACCAGCCGACCAGGCCAGCCCGCTGCTCCnGGTCTTCTGTGCTCGTGTGTCCCTCCCACCACGCTGAACTCACTGGGCTCCTCCCAGGA | G | A |
| 819 | CCCCTCTCCTTACCTGGACGTGGCAGCCAACCAGCCGACCAGGCCAGCCCGCTGCTCCAnGTCTTCTGTGCTCGTGTGTCCCTCCCACCACGCTGAACTCACTGGGCTCCTCCCAGGAC | T | G |
| 820 | CCAGGACTCTCGGTCTCAAGCAGCAGTCGTGCACAAGTGTGCTGTTGTCACCTCTGAAAnCTAGTGAATGACCCCACACACTCGGACCTCACTGCATAGCAGGAGTCGTCCCACTGATA | G | A |
| 821 | GTTGTCACCTCTGAAAACTAGTGAATGACCCCACACACTCGGACCTCACTGCATAGCAGnAGTCGTCCCACTGATACCTTCCTACCCCCGTTTCTCTCAATCCTGTGCCCATAACACAG | A | G |
| 822 | CACTGATACCTTCCTACCCCCGTTTCTCTCAATCCTGTGCCCATAACACAGCAAGTGTTnAATAAACCAATGAAGAGCGAATGATTTCTTCTCTGGTGCAGGGCAGCGGACAGGGATGA | A | C |
| 823 | TCAATCCTGTGCCCATAACACAGCAAGTGTTCAATAAACCAATGAAGAGCGAATGATTTnTTCTCTGGTGCAGGGCAGCGGACAGGGATGACCTGGGCTCAACACCCTGCCCAGGGGCC | T | C |
| 824 | CACAGCAAGTGTTCAATAAACCAATGAAGAGCGAATGATTTCTTCTCTGGTGCAGGGCAnCGGACAGGGATGACCTGGGCTCAACACCCTGCCCAGGGGCCTGGCGTGTCAGCAGCCAG | C | G |
| 825 | CTGGTGCAGGGCAGCGGACAGGGATGACCTGGGCTCAACACCCTGCCCAGGGGCCTGGCnTGTCAGCAGCCAGGAGACCTCTTCCAGAGTTGTAAAGGCAAACACTAAATAGCCCCGGT | A | G |
| 826 | ACCCTGCCCAGGGGCCTGGCGTGTCAGCAGCCAGGAGACCTCTTCCAGAGTTGTAAAGGnAAACACTAAATAGCCCCGGTCCTCTGAGGTGGGAAAACGGGCTTGGGACACAGGTAGCC | G | C |
| 827 | GGGGGCCACTGTCCTGGCCCAGCTGTGGTCCTGGGAGGTGGCTCACTGTGACAACCCCAnGTGACGAAGAGAGGGCTGAAGATTTTATTCCCATGTTTGAAGCTGGAGACATTCAAGCT | C | T |
| 828 | AGATTTTATTCCCATGTTTGAAGCTGGAGACATTCAAGCTTCGGCTGAAGCCACTGACCnTTCAGTGAGTTCCCTCCTTGGGCTGGCACTTTGCTGGGCCCTCAGGCGGGACTGGGAGC | A | C |
| 829 | ATTCCCATGTTTGAAGCTGGAGACATTCAAGCTTCGGCTGAAGCCACTGACCCTTCAGTnAGTTCCCTCCTTGGGCTGGCACTTTGCTGGGCCCTCAGGCGGGACTGGGAGCAGGGAGG | A | G |
| 830 | CTTGGGCTGGCACTTTGCTGGGCCCTCAGGCGGGACTGGGAGCAGGGAGGGAGACTGGAnTTTGTCCTTGAAGAACTAAGAACAAAGAGGGCAGAAGCAATGTACTCACTCAAGGCAGC | A | G |
| 831 | GGGCCCTCAGGCGGGACTGGGAGCAGGGAGGGAGACTGGAGTTTGTCCTTGAAGAACTAnAACAAAGAGGGCAGAAGCAATGTACTCACTCAAGGCAGCCCTTGCTCTCCAACAACTCG | AG | A |
| 832 | CCCCGTGCCAATAGAGCTCAGAGGTCCTTGGAGAGGGTTTTCACGAAGGACTCACGTAGnATGCATGGTCCTCATGCTCAGCTTTTGACCTGCAGCCCCACGACAAACTGCTTTCGTTG | A | G |
| 833 | CGTGCCAATAGAGCTCAGAGGTCCTTGGAGAGGGTTTTCACGAAGGACTCACGTAGGATnCATGGTCCTCATGCTCAGCTTTTGACCTGCAGCCCCACGACAAACTGCTTTCGTTGGCT | C | G |
| 834 | TTGACCTGCAGCCCCACGACAAACTGCTTTCGTTGGCTCAGGTCTACAGACGGCACTGCnGCACACCCTGCTGTGGGCCAGAAACCTGGGAGTGACTCCCCAGCCCTCACTGCCCCCTC | C | T |
| 835 | TGGGCCAGAAACCTGGGAGTGACTCCCCAGCCCTCACTGCCCCCTCTAAACACCTCCCCnTAGTTAGTCATTCTTGAAAATATACCTAAATTCTTCTTGCCGCTCTTACTACACTTATT | G | C |
| 836 | CCTCTAAACACCTCCCCCTAGTTAGTCATTCTTGAAAATATACCTAAATTCTTCTTGCCnCTCTTACTACACTTATTCACAGGAGGTACTATCTTCCCTTGCCTAGGGGGCAGCAGCCT | A | G |
| 837 | CCTTCAAGGCCCTGTGCTCCTTGATCCTCACCCCCCATCCCCCCTCAGCCACTACCAGAnATTCTCTCAGACCCTTAGGCACACCAGAGGCTCGCCAGCTTTTGTGATCTGTGCTTAGG | T | C |
| 838 | ACCCTTAGGCACACCAGAGGCTCGCCAGCTTTTGTGATCTGTGCTTAGGCTCTCCCTCTnGGAACGCTGCCCACGCCCCCCTCCCCACCCAACTGGCTAACCCAATCATCTAAGCAAGC | C | T |
| 839 | CAGAGGCTCGCCAGCTTTTGTGATCTGTGCTTAGGCTCTCCCTCTTGGAACGCTGCCCAnGCCCCCCTCCCCACCCAACTGGCTAACCCAATCATCTAAGCAAGCTCCCTTCTCAGTCA | T | C |
| 840 | CCCTCTTGGAACGCTGCCCACGCCCCCCTCCCCACCCAACTGGCTAACCCAATCATCTAnGCAAGCTCCCTTCTCAGTCAGGGCTTCCTGAATAACCAAGCTCAGCTAGTTCTCTCCAG | T | A |
| 841 | CAATCATCTAAGCAAGCTCCCTTCTCAGTCAGGGCTTCCTGAATAACCAAGCTCAGCTAnTTCTCTCCAGTTATGCTTACCCAACACCTGTCCTTTGCCCTTCAGGGCACTGATCACAA | C | G |
| 842 | CAGGGCACTGATCACAATTAGGAATTTAATGCTCATCTTCTCCAGGAGACCAAGTTCTAnATCTTTTGTGTACAAATGAATGACTGAATATATTAGGCACTCTAAAAGACAAGTTACCT | C | CA |
| 843 | GACTGAATATATTAGGCACTCTAAAAGACAAGTTACCTCCACAAGTATTATCGTCAATCnGCACCGGAAATCCCAGAGCCAGGATTTGAGGGAAGGTCCTTCACTCCAAGCACATTTGA | A | T |
| 844 | TACAGACATGTCTCAGGCAGAAAAATAAGGAAGTCTTCACAAGGAATTCCTTTTTAGTTnGGCCATTTCAGTAAGTACCAAAGAAACATCTAAATGTCTGCCCCCACAGCATACTGGTC | C | G |
| 845 | GACTAATCCAGCTGTATGTGGAGGCTGTGTAGGGGAATGATGAAAACTACCCAGGTGGAnTCCACTTATGGAAACTTGAAAGCCAAGATAAATGGGAACTTAATCCAAGAATGGGAAGC | G | A |
| 846 | CAGGTGGAATCCACTTATGGAAACTTGAAAGCCAAGATAAATGGGAACTTAATCCAAGAnTGGGAAGCCACATGAACTCCAGGGGAACTGGAGACCTATTTGTAACAGGATATTCACCT | C | A |
| 847 | CTCCAGGGGAACTGGAGACCTATTTGTAACAGGATATTCACCTTTCCTGATCCACTTCTnTAAGGTGGTGACTAAGAATTCTAAGGCTAGGCAAGGACCTGAGAGGCAACTTGACTGGA | G | C |
| 848 | CATGAATACTGCTCCCCTGGAGCTACACAAGGTGGAGGTCCTCCTCTGGGCACATCTGGnTTCCAATCACAGGCAATCATTCACCAAAATAAAACTACAAGGCCCAACTGTGAAGGCAG | T | C |
| 849 | ATTAATTAACCCAAGTTCAGAAATGACTTCAAAGTCTGTTTCCTAAACCGTATACAAATnTGTGCGTATCTGCATTTATGTACACTTTTCTTTTAAAAAACCCAATACTTTTATTAGAT | A | G |
| 850 | TAACCCAAGTTCAGAAATGACTTCAAAGTCTGTTTCCTAAACCGTATACAAATGTGTGCnTATCTGCATTTATGTACACTTTTCTTTTAAAAAACCCAATACTTTTATTAGATTCTCAA | A | G |
| 851 | TCCTAAACCGTATACAAATGTGTGCGTATCTGCATTTATGTACACTTTTCTTTTAAAAAnCCCAATACTTTTATTAGATTCTCAAAAGGTCAGAAGGAAAGGTCCTTTACTCTCAAGTC | T | A |
| 852 | CTTTTAAAAAACCCAATACTTTTATTAGATTCTCAAAAGGTCAGAAGGAAAGGTCCTTTnCTCTCAAGTCCTTTTTTCACTTACACACTGCTATGTATCTGTATGTATATCTAACCATC | T | A |
| 853 | CCCAAAATAGTCAGTGGTTTAAAGTACTTCCAGGGACTTCCCTGGCAGTCCAATTGTTAnGACTGTGCACATCTACTGTAGGGGTCATGGGTTTGATCCCTAGTCAGGGAACTAAGATC | T | A |
| 854 | CAGGGACTTCCCTGGCAGTCCAATTGTTAAGACTGTGCACATCTACTGTAGGGGTCATGnGTTTGATCCCTAGTCAGGGAACTAAGATCCCATATGCCACTCAGCATGGCCAAAAAAAT | A | G |
| 855 | TCCCTGGCAGTCCAATTGTTAAGACTGTGCACATCTACTGTAGGGGTCATGGGTTTGATnCCTAGTCAGGGAACTAAGATCCCATATGCCACTCAGCATGGCCAAAAAAATAAAACAGT | T | C |
| 856 | TGTGCACATCTACTGTAGGGGTCATGGGTTTGATCCCTAGTCAGGGAACTAAGATCCCAnATGCCACTCAGCATGGCCAAAAAAATAAAACAGTACTTCTCATGATTCTCTAGGGTGGC | C | T |
| 857 | TCCCATATGCCACTCAGCATGGCCAAAAAAATAAAACAGTACTTCTCATGATTCTCTAGnGTGGCAACCCTTAGCTGGAGGATTCTTTAGTTTCAGGAGGTCAGCAGGGTAGAGGGTCT | C | G |
| 858 | TATGCCACTCAGCATGGCCAAAAAAATAAAACAGTACTTCTCATGATTCTCTAGGGTGGnAACCCTTAGCTGGAGGATTCTTTAGTTTCAGGAGGTCAGCAGGGTAGAGGGTCTGGATT | C | CA |
| 859 | TCTCTAGGGTGGCAACCCTTAGCTGGAGGATTCTTTAGTTTCAGGAGGTCAGCAGGGTAnAGGGTCTGGATTTTTGACACTTAAGTTTCTGGTCTGAAAATGTCAGGTTTCCAACTGCA | C | G |
| 860 | GGATTTTTGACACTTAAGTTTCTGGTCTGAAAATGTCAGGTTTCCAACTGCAAAAGTACnAACAGCTAAAATATCACTTCTGGCACCTTTGATTAGTGAGAAGTCCAGCCCACACCCTT | G | A |
| 861 | CACCCTTAAGGAAAATTAGCTCTTATTGGAAGGAGCAGCACTCATATACAGAGATGGGAnTGTTGGTGACCATTTCTGGAGGTGACTTACCACACCTTTTTTTTGAGATAAGGGTATAG | C | T |
| 862 | AGATTTCTAAAAACTCTGCATATGATTGAATAAAACGTCAGTGGTTTCCAAAACATGTAnCGAGTTAAGAAACACTGGTTTTGTGAATGGACATGACTGGTCCAGGGTAAAGATGTTCT | C | T |
| 863 | GGTCCAGGGTAAAGATGTTCTCACACAGAGCATCCAGGGGGAGCAGCGGAGCCAAAGCTnTACGACAACACAGGTTATTTTCTCCACTCGGGCACAAATCAACAAACCCCAAACCCCAG | A | G |
| 864 | TCCAGGGTAAAGATGTTCTCACACAGAGCATCCAGGGGGAGCAGCGGAGCCAAAGCTGTnCGACAACACAGGTTATTTTCTCCACTCGGGCACAAATCAACAAACCCCAAACCCCAGAA | G | A |
| 865 | GGACCTCACTTAATGGGACCTGTCACCTGGGCCTGCACAAACCATGCACAAGCCGAGGGnTGAGTGAAGGCTGGCAGGTGGGGCTCCCGTCTACCTTTTCACTGGAGCTTTCTCAAACC | A | C |
| 866 | AACTGCTCTTCAAAGGAGAAAAAGGCAGACATGTTTATGCCTAGTGACTAATATACTTCnAAAAAAACTGCTTTTCACTCATTTCATATTCCAGAAGGAATGCATGATGGAAGCAAGTT | C | CA |
| 867 | GAATCATCTTGCCATATAAAGGGCACACCATATTCTTAAAAACTACTTAATGTGACTCAnTGTTCCCCCTAGTGAGAAACAGGAAAGCAGGCACCGAGGCATCAGATGACTCACAGTAG | T | C |
| 868 | GGCACACCATATTCTTAAAAACTACTTAATGTGACTCACTGTTCCCCCTAGTGAGAAACnGGAAAGCAGGCACCGAGGCATCAGATGACTCACAGTAGTTTTGTCAGAAGCATGGGATC | G | A |
| 869 | TTCCCCCTAGTGAGAAACAGGAAAGCAGGCACCGAGGCATCAGATGACTCACAGTAGTTnTGTCAGAAGCATGGGATCTGGACTCAAGAGAAGCAACGCTTTCATTCTTTTTCTATAAC | C | T |
| 870 | AGAACCAGAATGAGGCACATCTCTGTATCCTGGTTTAGGTCTCTGACCTACAAATGCTGnCTGCTGCTGCTGCTGCTAAGTCGCTTCAGTCGTGTCCGACTCTGCGTGACCCCATAGTC | C | CCTG |
| 871 | CTACAAATGCTGCCTGCTGCTGCTGCTGCTAAGTCGCTTCAGTCGTGTCCGACTCTGCGnGACCCCATAGTCGGCAGCCCACTAGGCACCCCTGTCCCTGGGATTCTCTAGGCAAGAAC | C | T |
| 872 | CAGTCGTGTCCGACTCTGCGTGACCCCATAGTCGGCAGCCCACTAGGCACCCCTGTCCCnGGGATTCTCTAGGCAAGAACATTGGAGTGGGTTGCCATTTCCTTCTCCAATGCATGAAA | T | TGGGAT |
| | | | TCTCTA |
| 873 | CATTTCCTTCTCCAATGCATGAAAGTGAAAAGGGAAAGTGAAGTCGCTCAGTTGTACCCnACTCTTAGCGACCCCATGGACTGCAGCCTACCAGGCTCCTCCGTCCATGGGATTTTCCA | G | A |
| 874 | CCCCATGGACTGCAGCCTACCAGGCTCCTCCGTCCATGGGATTTTCCAGGCAAGAGTATnGGAGTGGGTCGCCACTGCCTTCTCCAAATGCTGCCTACAGGAGTAACTAAACTCAATTC | A | T |
| 875 | GAAGCAATCAGCACATGCTGAGGCTTTTTTCCTCTAGAAATGGTTGGTGCTTCCACTTAnGGAGTCTCAGAGATGCAGGAATGACTCGAGACTTTTGCTCTTCTAACGAAGGAGCCTAA | C | T |
| 876 | TTCCTCTAGAAATGGTTGGTGCTTCCACTTATGGAGTCTCAGAGATGCAGGAATGACTCnAGACTTTTGCTCTTCTAACGAAGGAGCCTAAGAAGCTAATCTTCTTTTGTACAGGTACA | A | G |
| 877 | TTTAAAGCAGAGAAATCTTCCTTTGATTTCTAGGTGAGGAAAGAGGGATGACAATCACCnAAGACTTGTGATCTGCACTGTCTATCCACTATCATTGAGGTCCACATTACCTTCCTCAA | A | G |
| 878 | GGATGACAATCACCGAAGACTTGTGATCTGCACTGTCTATCCACTATCATTGAGGTCCAnATTACCTTCCTCAATTTTTCCAAAATGGGAGAGACAAGAGAATTTCCCTTTGATGCAAA | T | C |
| 879 | TGTTCCCACTTCCCACAGCTATCCCACCTAGCCTTACCTCCTGCTGGGTCTTCAAATGGnTAACAGAAACTACTCACAGCTACGCATACCTGTTAGCTGGAACCAGAGTGTGACACACA | A | G |
| 880 | AAATGGGTAACAGAAACTACTCACAGCTACGCATACCTGTTAGCTGGAACCAGAGTGTGnCACACAGAATTTACTCCAGCAACTAGACAAGCTCAGAGTTAAGCACTCCCGCCAGATAG | G | A |
| 881 | AGAGTGTGACACACAGAATTTACTCCAGCAACTAGACAAGCTCAGAGTTAAGCACTCCCnCCAGATAGCCTGCATCAGAGGAAAAAACAACTTTCCCCACCCACAAATCATGTCCCTGA | T | G |
| 882 | AAACCTCAAGTGCATTTATTAGTGGTAGGCCAGCGGCACCATTTCACACTCAAATGAGAnTTCCTGGGTGAAACAAATTTCAGCAGAGAACATGCTTTATTGAAGAGTAGATATAGAAC | A | G |
| 883 | CAAAAGCTGCCCCAAGATAGCAGGTTATAATGGGCTAGAGAAATTAGAGGGAGCATCTCnTCCTTCACTTAAACGACACCAAAAATAGAGGAGACCAGAGAATGGGGGGTGGTGGTAAA | C | T |
| 884 | GAAATTAGAGGGAGCATCTCTTCCTTCACTTAAACGACACCAAAAATAGAGGAGACCAGnGAATGGGGGGTGGTGGTAAATCCCAAAAAGGAAATGGAGTTCTTGGAAGGGCATAAGCA | G | A |
| 885 | TTTCATTCCCTTTCACCAAGATGTTGCCCACTGTTCACAGGTGAACTCAAATCCAAATAnAGAATCACCACCTCTCAATTCTTTTCTCCATCTCATTTAGAGCATATGCTGTAACCAAG | C | G |
| 886 | GTAACCAAGACTAATCCCTGCAAGAGTCTTCAGAAGTATTTGGCTCTGGTCAGCCTGATnCTGATTAAAAACAGGTCTTTCAGTAAAAATGGAGGAAATAACTTAAAGTGAGATGAAAT | G | A |
| 887 | CATGGCCCACCCTTGGGCCTGTGGCTCTGGGCAGTGGCCAGTGCGTGACCATAGTATTTnCCCCACAGCATGCTGAATGCAGCTTAGGAGGGAAACAGGCCAGAGCTGCAAATAGGGTG | A | T |
| 888 | ACTACCCTGCAAGCTTTGCGTGGTTAATACCATAGCTACTCTAGTCAATATCTCTAGATnTTTTTCTCAAGTTTTGCTTCTTGGGAGATTTATCAGATTTGTCGAGATTCTACCAATGA | T | G |
| 889 | ATTTCATCTGCGTAGAATCCTACAGTTTATACTCTTTTGTGTCTGGTTTCTTTGGATATnTATTTGAATTTTCAGATTTTCTATTTTGTGCACGTGTCACTTGGTAATTTGTGTCTTTC | T | A |
| 890 | GGCAGCTGCCTGTGGGGCTGGATGCATAGGTGGCCCCAGCATTTGCTGTGCTCCTCTGGnTCCGAGGCCACATATTTCCTGGGGTTCCTCACCCTCGTGTCTGCTCGCTGCTTCCCAGC | T | C |
| 891 | GTGCTCCTCTGGCTCCGAGGCCACATATTTCCTGGGGTTCCTCACCCTCGTGTCTGCTCnCTGCTTCCCAGCAGGGTAGGGAGCTGGTCTGGACCTTGAAAGTGGCACTGCTCCCTTCT | T | G |
| 892 | CTGAGTGATCACCCCACAGAGACCAAGGCAGCACCGCCAGGCAGGCTGCTAGGGCAAACnCAGCCACAGCCACAGGGACAGAAGATTCACCTTGGGAGTGGGCTACACTGCAACTTTGA | G | A |
| 893 | ATCTTTTACTTTTTTTTCCTTGTGTTATTTCCCCTGACTGAACTCTTTATTATGATGTTnATCATAGCTGACTTTCTTCCTGATCCCGGAGAAGAATTTTCAACACTTTACTTTTAAGT | G | C |
| 894 | GTGTTATTTCCCCTGACTGAACTCTTTATTATGATGTTGATCATAGCTGACTTTCTTCCnGATCCCGGAGAAGAATTTTCAACACTTTACTTTTAAGTTTAATTGTTACTTTTTTGTAT | T | C |
| 895 | TATTCCATTTGTTTTGAGACTGTATTATATTGCCTGTTGGAATTGCTTTAATCTGTCTTnTTGATAGGTCTTTTATCATCCAAGGTACAATTTTTCTTGTTGTTGGTATAACGAGTGAT | T | C |
| 896 | AAAACAATCTTCTGTGGCTATAAATACTTTTGAAAGGACAGCTTTAGATGCACTCCTTAnGTTCTGATAAGCCATGCTTTTATGGTAATTCGGCTCAAAGTAATTTCTAATAGCCACTG | C | T |
| 897 | TGAGCCAAGGCCTTTCTCTGATGTTCCCTTTGCCATCTCACCTCCATATGCCCCCTTCAnGCCACATGCCAGGCATGTGCTGGAGGCACTGCTGTGCGGTCAGCGTGGCTCCAAGGCCA | T | C |
| 898 | ACCTGCTTCCCTCGGGGCCTTGGACACCACTGGCTCTTCCTGGGCCCCTGCCCGGCACAnAGGCAGCCACACACATTCTCCTTTTTCCCTTTCTCTTTTCCCCACACCCCACCCCGTTC | A | G |
| 899 | GTACCGCCTGGGCAAGGGGGCTCAGAGCCTGGGTGTTTTTCCCGGGACTACCTCCTGGCnCACAAGCTGAGACCCCAAGCAGCCACTGCTCCTGCCTTTGGCCTCCTTTGATTTCTGCA | C | T |
| 900 | GCTCCTGGAGCTGGGAAACCGGCTGCGAGCGGAGCTGGGGCACCTTACCGGTGGGTCATnCCCCCCACTCCGCCCCTGCTCTGGGGATGGAGGTGCAGGGTGCAGGGCAGGGCTGGCCC | TC | T |
| 901 | AGTGTGCGAGCAGCAACCGAGGGCGTCTTTCACGGGAGCTGGCTCCTTCCCTTGGGACCnGTTTGACAAAGCCCTGCTTTCGTTCAGTCCTCCTTCTGTTCCACAAGTTGAAGGGGAAG | T | C |
| 902 | GTGTGCGAGCAGCAACCGAGGGCGTCTTTCACGGGAGCTGGCTCCTTCCCTTGGGACCTnTTTGACAAAGCCCTGCTTTCGTTCAGTCCTCCTTCTGTTCCACAAGTTGAAGGGGAAGG | G | A |
| 903 | GTCATAGAGTGGGGATCACACAATATGTATTCTCTGATACCTAACATTTCTTAAAGGCTnTTTTTTTTTTTCCCTTGATGTTGAAAATGCTATTTGCTCACTATAAAAATTTCAATGAA | C | CT |
| 904 | AGAGGCAGTACCCTGACAGCTCCTCAGGATGTGCGGTAGGCAATGCAGTTTCCATAGCTnTTTTTTTTTTTCCCTTTAGTTTCTTAGCTTTTGATACAATTCATGTGAAAGATTATTCT | G | GT |
| 905 | CCTTTTTTCTTGAGGTTTCCAGAATATCCTGAGGCTCCTGGACCTGCCAGGAGATGAACnTTCTTATCCCCTCATGAGGCTGGCACCTGGCAGTTTTCCTGGGAGGGCCTTATGAGCCT | T | A |
| 906 | AAGTTGGGAGGAGCTGTTAAAAACAGAGTCCATTGGAACAAATTGGAACTTCAAAGCACnGTGGCTTTTATTGGCTGGGCCATTGCCAGGCAGAGATAAAATCCTTCTCCAGCTGCCAA | A | G |
| 907 | AATGGGTCAGGGGGACAGAGAAGCCTAGGGAGGCAGTGTGCCAGACCAGGAGACACTGGnCTGAGAAACAGAAAGGCTGGCTCTGAGAGGGGCCCAGGAGGCACAGTGTTTTCCACAAC | C | G |
| 908 | CTGTTTTCAGACGTGGGAAAATAGGTAGCAGAGGACCGGGATCCTTGAACGAGGAAGACnAACGAGGCGCCTGTCGGGCCCCCTGGCTTTCTGCCCCGAGGCATCTTCTGGGCTCAGTC | A | G |
| 909 | GCCAGGCCCCTCTGTCCATGAGATTCTCCAGGCAAGAATACTGGAGTGGGTTGCCATTTnCTCCTCCACGGGATCTTTCTGACCGACCCAGGGATCGAACCGAGGTCTCCTGCGTTACA | C | T |
| 910 | TTCTTGTTCCCAAGTTCGTTCTGTTCCTTTCTCTCCAGTTTCTCCCCCTGAAGGCTAGTnCATCTCCTGGTAGATGAGATGTTAGTATTTCCAGATAAGTGTATTGGCAGCCGCTTGAG | T | C |
| 911 | GAGCACCCAGCACCGACAGCCTGTAGCCTCCAGCACCCTGAGGGGAGCCCCACTGTGACnTGGGCAGCCTTTGGCGTTGAAGGGATGAAGATGGAAGCCCAGACAAGGGGCAAGCCTAC | G | C |
| 912 | TTTCTGACCTCCCAAGGGTCTATCAGTGGTCCCAGGAGATAGTAAGGGCCGTGCAGGGCnTGGCCACCAGTCAGTCCTTCCTGGGCTCACGTGATTCCCGATCCGTGTTGTTTTCAGTC | C | G |
| 913 | GGCCTTGGCACTGTTGAGGAGACCAAGCTGTGGTTCTGGCCTGGAGGGCAGCTCGAGATnCTGAGCGCAGAGCCAGCTAGTCAGTGTGGGCACTTCTTCCCACCCCCACATTTGCCCTT | A | G |
| 914 | TGCACACCAGAGGAATCCCAGACATGTCCTGTCACCCCAGTTTCCTCACTCCCCATTGCnTGGCTGATCTTGGAGGCGAGGGCAGGACACCAGGTGCCAGGCAGAGCAGGAGGCAGGAT | G | A |
| 915 | GACCCTTGAGTGTGACAGGGTCCTGGACAGGCACTGCCTGGGGGGAGGGGCGGGGAGGCnGAGGCCTTCCCACTGCCCAGGCCCCAGCCGGAGACCTTGGATGCTGCTCTCCAGGGGCT | G | A |
| 916 | AGACAAAGAGATGGCTGGATGGCATCACTGACTAGATGGACGTGAGTCTGAGTGAACTCnGGGAGTTGGTGATGGACAGGGAGGCCTGGCGTGCTGTGATTCATGGGGTCGCAAAGAGT | T | C |

The column in Table 2 labeled “flanking sequence” provides the sequence information for a reference nucleotide sequence for identification of the polymorphism within the genome of a cattle, such as Norwegian Red cattle. The sequences SEQ ID NO: 1 to 916 are each polymorphic sequences including a polymorphic site (“n”). All or only part of the polymorphic sequence flanking the polymorphic site can be used by the skilled practitioner to identify the polymorphism within the genome of a cattle, such as Norwegian Red cattle. It is to be understood that the information provided in table 2 is a supplement to the information provided in table 1, i.e. P #1 in table 2 and P #1 in table 1 refers to the same polymorphism.

According to particular embodiments, the at least one polymorphism of the invention is selected from the group consisting of P #1 to P #916. According to particular embodiments, the at least one polymorphism of the invention is selected from the group consisting of P #1 to P #310. According to particular embodiments, the at least one polymorphism of the invention is selected from the group consisting of P #13, P #15, P #21-P #32, P #46, P #47, P #54, P #55, P #242-P #247, P #251, P #252, P #261-P #267, P #269, P #311-P #332, P #334-P #343, P #346-P #482, P #595-P #602, P #604-P #608, P #611, P #615, P #616, P #622-P #625, P #627, P #629, P #633-P #667, P #669-P #677, P #679-P #681, P #684-P #687, P #689-P #766, P #768-P #886 (C4:0). According to particular embodiments, the at least one polymorphism of the invention is selected from the group consisting of P #33, P #241-P #248, P #303-P #312, P #314-P #344, P #346-P #475, P #477-P #481(C16:0). According to particular embodiments, the at least one polymorphism of the invention is selected from the group consisting of P #19, P #34, P #38, P #39, P #141, P #148, P #153, P #233-P #240, P #242-P #246, P #271-P #278, P #280-P #283, P #285, P #289-P #302, P #311-P #334, P #339, P #340, P #343-P #475, P #477-P #481, P #484-P #494, P #497, P #543, P #588, P #589, P #916, P #911, P #14, P #37, P #903, P #7 (C18:1). According to particular embodiments, the at least one polymorphism of the invention is selected from the group consisting of P #1, P #12, P #15-P #17, P #24, P #41, P #63, P #64, P #69-P #71, P #74-P #79, P #83, P #85-P #87, P #89-P #93, P #97-P #103, P #112-P #118, P #120, P #121, P #123-P #135, P #138-P #141, P #148, P #153-P #168, P #218, P #220-P #226, P #249, P #250, P #253-P #260, P #267, P #495, P #499, P #501, P #503-P #514, P #517-P #539, P #541, P #542, P #544-P #547, P #554-P #585, P #590-P #592, P #595-P #597, P #599-P #614, P #617-P #621, P #623-P #652, P #654-P #669, P #672-P #674, P #676-P #678, P #681, P #682, P #688-P #744, P #746-P #769, P #771-P #813, P #815, P #818-P #824, P #826-P #831, P #872 (C6:0). According to particular embodiments, the at least one polymorphism of the invention is selected from the group consisting of P #1, P #10-P #12, P #16-P #19, P #40-P #45, P #48, P #49, P #51, P #56-P #60, P #63-P #66, P #68-P #93, P #97, P #99-P #101, P #103-P #108, P #111-P #118, P #120, P #121, P #123-P #218, P #220, P #224-P #231, P #254, P #258, P #259, P #268, P #270, P #285-P #288, P #302, P #495, P #496, P #498-P #539, P #541-P #551, P #554-P #586, P #590-P #594, P #628, P #682, P #683 (C8:0). According to particular embodiments, the at least one polymorphism of the invention is selected from the group consisting of P #3, P #8-P #10, P #16-P #20, P #49-P #51, P #56-P #62, P #64, P #69-P #72, P #74, P #76-P #78, P #80-P #96, P #104-P #109, P #111-P #115, P #117, P #118, P #120, P #121, P #127, P #128, P #132, P #133, P #136-P #142, P #148-P #153, P #156-P #166, P #224-P #226, P #285-P #288, P #297-P #302, P #490-P #494, P #501-P #514, P #517-P #539, P #541-P #547, P #551, P #554-P #580, P #585, P #586, P #589-P #593, P #628, P #889-P #891, P #899, P #900, P #902-P #916 (C10:0). According to particular embodiments, the at least one polymorphism of the invention is selected from the group consisting of P #3, P #9, P #10, P #16-P #20, P #49-P #53, P #61, P #62, P #64, P #67, P #69-P #72, P #74, P #83, P #85-P #87, P #89-P #96, P #104-P #113, P #115, P #117-P #122, P #127, P #128, P #132, P #133, P #138, P #139, P #141, P #142, P #148-P #151, P #153, P #219, P #224-P #226, P #276, P #285-P #288, P #290-P #302, P #487-P #495, P #497, P #501-P #515, P #517-P #547, P #551, P #554-P #580, P #585, P #586, P #588-P #594, P #887-P #891, P #893-P #916 (C12:0). According to particular embodiments, the at least one polymorphism of the invention is selected from the group consisting of P #9, P #10, P #17-P #19, P #34, P #69, P #70, P #83, P #85, P #117, P #120, P #121, P #127, P #128, P #132, P #133, P #139, P #141, P #148-P #151, P #153, P #224-P #226, P #275, P #276, P #279-P #281, P #284-P #302, P #483-P #494, P #497, P #501-P #514, P #517-P #539, P #541, P #543-P #580, P #584-P #594, P #889-P #892, P #899, P #900, P #902-P #916 (C14:0). According to particular embodiments, the at least one polymorphism of the invention is selected from the group consisting of P #243-P #246, P #314-P #332, P #334-P #340, P #346-P #352, P #354-P #363, P #366-P #392, P #394-P #396, P #398-P #410, P #412-P #475, P #477-P #481(C14:1 cis-9). According to particular embodiments, the at least one polymorphism of the invention is selected from the group consisting of P #4, P #6, P #15 (CLA). According to particular embodiments, the at least one polymorphism of the invention is selected from the group consisting of P #2, P #5 (DHA). According to particular embodiments, the at least one polymorphism of the invention is selected from the group consisting of P #3, P #8-P #10, P #16-P #19, P #44, P #49, P #51, P #56-P #60, P #64-P #66, P #68-P #78, P #83, P #85-P #87, P #89-P #93, P #104-P #108, P #111-P #115, P #117, P #118, P #120, P #121, P #127, P #128, P #132, P #133, P #136-P #153, P #156-P #167, P #170, P #177, P #180, P #181, P #224-P #226, P #231, P #232 (DNS). According to particular embodiments, the at least one polymorphism of the invention is selected from the group consisting of P #34, P #39 (MUFA). According to particular embodiments, the at least one polymorphism of the invention is selected from the group consisting of P #36 (NEFA). According to particular embodiments, the at least one polymorphism of the invention is selected from the group consisting of P #6, P #34, P #35, P #38, P #39 (SAT).

It is understood that the foregoing disclosure regarding the polymorphisms of the invention is applicable to the following aspects.

Method for Selecting a Cattle

The present invention provides in a first aspect a method for selecting a cattle, such as Norwegian Red Cattle, which possesses a genotype which in a female milk-producing cattle is indicative of a desired milk fatty acid composition, the method comprising:

determining the presence of at least one allele, such as at least two, three or four alleles, which in a female milk-producing cattle is indicative of the desired milk fatty acid composition, within the genome of said cattle; and

selecting said cattle when the at least one allele, such as the at least two, three or four alleles, which in a female milk-producing cattle is indicative of a desired milk fatty acid composition, is present.

It is to be understood that the term “desired fatty acid composition” as used herein does not necessarily refers to a specific fatty acid composition in milk but rather increase or decrease in the amount of certain fatty acids.

The genotype referred to in the method according to the first aspect of the present invention refers to that part of the genetic make-up of a cattle which determines a specific phenotype, i.e. milk fatty acid composition, only in female milk-producing cattle. Said cattle may be male or female, but the genotype referred to will only determine the specific phenotype in female milk-producing cattle.

A female milk-producing cattle selected by the method according to the first aspect of the present invention will have a desired milk fatty acid composition. A male cattle selected by the method according to the first aspect of the present invention possesses a genotype which in a female milk-producing cattle is indicative of a desired milk fatty acid composition and may therefore find use as a breeding animal or as a producer of semen or sperm which may find use in various breeding programs.

Since the genotype referred to in the above method determines a specific phenotype only in female milk-producing cattle, it has been decided to differentiate between a female cattle, which not necessarily produces milk, and a female milk-producing cattle, which produces milk by definition.

The cattle referred to in the above method may be male or female. In one embodiment according to the present invention, said cattle is male. In another embodiment according to the present invention, said cattle is female, preferably a female milk-producing cattle.

In one embodiment according to the present invention, said cattle is Norwegian Red cattle.

In one embodiment according to the present invention, said female milk-producing cattle is female milk-producing Norwegian Red cattle.

A female milk-producing individual having a desired milk fatty acid composition has a higher probability of a desired milk fatty acid composition than a random female individual (under the same conditions) with whom it is comparable. Two female individuals are comparable if they are, with regards to all discriminating factors except the genotype at the polymorphic site which is used for predicting milk fatty acid composition, random representatives of one and the same population of female cattle.

In one embodiment according to the first aspect of the present invention, the at least one allele is an allele of at least one polymorphism, such as at least one single nucleotide polymorphism (SNP). In a preferred embodiment, the at least one polymorphism is selected from the polymorphisms listed in table 1.

The polymorphism of the present invention can be present in either of two forms, i.e., the polymorphisms have a total of two alleles. The at least one allele referred to above is herein meant to refer to one of the two alternative forms of the polymorphism, i.e. one of the two alternatives that is present at the polymorphic site. Said in other words, P #15 has a total of two polymorphism alleles (A/G), i.e. there may be an adenine at the polymorphic site or a guanine at the polymorphic site. An adenine at the polymorphic represents one polymorphism allele while a guanine at the polymorphic site represents the other polymorphism allele. A polymorphism allele referred to as a “fat allele” for a specific trait is indicative of increased amount of that trait in milk while a polymorphism allele referred to as a “non-fat allele” for a specific trait is indicative of decreased amount of that trait in milk.

The knowledge provided in table 1 makes it possible to select a cattle possessing a genotype which in a female milk-producing cattle is indicative of a desired milk fatty acid composition. If it e.g. is desirable to reduce the amount of C16:0 in milk in order to provide milk with a healthier fatty acid profile, the at least one allele referred to above should preferably represent a “non-fat allele” for C16:0. One example of such an allele is P #33 where an adenine is positioned at the polymorphic site.

In another embodiment according to the first aspect of the present invention, the at least one allele is an allele of at least one polymorphism selected from the group consisting of P #1 to P #916, such as P #1 to P #310. In another embodiment according to the first aspect of the present invention, the at least one allele is an allele of at least one polymorphism selected from the group consisting of P #33, P #241-P #248, P #303-P #312, P #314-P #344, P #346-P #475 and, P #477-P #481 (C16:0), preferably the at least one allele represents a “non-fat allele” for C16:0. In another embodiment according to the first aspect of the present invention, the at least one allele is an allele of at least one polymorphism selected from the group consisting of P #19, P #34, P #38, P #39, P #141, P #148, P #153, P #233-P #240, P #242-P #246, P #271-P #278, P #280-P #283, P #285, P #289-P #302, P #311-P #334, P #339, P #340, P #343-P #475, P #477-P #481, P #484-P #494, P #497, P #543, P #588, P #589, P #916, P #911, P #14, P #37, P #903 and P #7 (C18:1), preferably the at least one allele represents a “fat allele” for C18:1. In another embodiment according to the first aspect of the present invention, the at least one allele is an allele of at least one polymorphism selected from the group consisting of P #1, P #12, P #15-P #17, P #24, P #41, P #63, P #64, P #69-P #71, P #74-P #79, P #83, P #85-P #87, P #89-P #93, P #97-P #103, P #112-P #118, P #120, P #121, P #123-P #135, P #138-P #141, P #148, P #153-P #168, P #218, P #220-P #226, P #249, P #250, P #253-P #260, P #267, P #495, P #499, P #501, P #503-P #514, P #517-P #539, P #541, P #542, P #544-P #547, P #554-P #585, P #590-P #592, P #595-P #597, P #599-P #614, P #617-P #621, P #623-P #652, P #654-P #669, P #672-P #674, P #676-P #678, P #681, P #682, P #688-P #744, P #746-P #769, P #771-P #813, P #815, P #818-P #824, P #826-P #831 and P #872 (C6:0), preferably the at least one allele represents a “fat allele” for C6:0. In another embodiment according to the first aspect of the present invention, the at least one allele is an allele of at least one polymorphism selected from the group consisting of P #1, P #10-P #12, P #16-P #19, P #40-P #45, P #48, P #49, P #51, P #56-P #60, P #63-P #66, P #68-P #93, P #97, P #99-P #101, P #103-P #108, P #111-P #118, P #120, P #121, P #123-P #218, P #220, P #224-P #231, P #254, P #258, P #259, P #268, P #270, P #285-P #288, P #302, P #495, P #496, P #498-P #539, P #541-P #551, P #554-P #586, P #590-P #594, P #628, P #682 and P #683 (C8:0), preferably the at least one allele represents a “fat allele” for C8:0. In another embodiment according to the first aspect of the present invention, the at least one allele is an allele of at least one polymorphism selected from the group consisting of P #3, P #8-P #10, P #16-P #20, P #49-P #51, P #56-P #62, P #64, P #69-P #72, P #74, P #76-P #78, P #80-P #96, P #104-P #109, P #111-P #115, P #117, P #118, P #120, P #121, P #127, P #128, P #132, P #133, P #136-P #142, P #148-P #153, P #156-P #166, P #224-P #226, P #285-P #288, P #297-P #302, P #490-P #494, P #501-P #514, P #517-P #539, P #541-P #547, P #551, P #554-P #580, P #585, P #586, P #589-P #593, P #628, P #889-P #891, P #899, P #900, P #902-P #916 (C10:0), preferably the at least one allele represents a “fat allele” for C10:0. In another embodiment according to the first aspect of the present invention, the at least one allele is an allele of at least one polymorphism selected from the group consisting of P #3, P #9, P #10, P #16-P #20, P #49-P #53, P #61, P #62, P #64, P #67, P #69-P #72, P #74, P #83, P #85-P #87, P #89-P #96, P #104-P #113, P #115, P #117-P #122, P #127, P #128, P #132, P #133, P #138, P #139, P #141, P #142, P #148-P #151, P #153, P #219, P #224-P #226, P #276, P #285-P #288, P #290-P #302, P #487-P #495, P #497, P #501-P #515, P #517-P #547, P #551, P #554-P #580, P #585, P #586, P #588-P #594, P #887-P #891, P #893-P #916 (C12:0), preferably the at least one allele represents a “fat allele” for C12:0. In another embodiment according to the first aspect of the present invention, the at least one allele is an allele of at least one polymorphism selected from the group consisting of P #9, P #10, P #17-P #19, P #34, P #69, P #70, P #83, P #85, P #117, P #120, P #121, P #127, P #128, P #132, P #133, P #139, P #141, P #148-P #151, P #153, P #224-P #226, P #275, P #276, P #279-P #281, P #284-P #302, P #483-P #494, P #497, P #501-P #514, P #517-P #539, P #541, P #543-P #580, P #584-P #594, P #889-P #892, P #899, P #900, P #902-P #916 (C14:0), preferably the at least one allele represents a “fat allele” for C14:0. In another embodiment according to the first aspect of the present invention, the at least one allele is an allele of at least one polymorphism selected from the group consisting of P #243-P #246, P #314-P #332, P #334-P #340, P #346-P #352, P #354-P #363, P #366-P #392, P #394-P #396, P #398-P #410, P #412-P #475 and P #477-P #481 (C14:1 cis-9), preferably the at least one allele represents a “fat allele” for C14:1 cis-9.

In another embodiment according to the first aspect of the present invention, a desired milk fatty acid composition is decreased amount of C16:0 in milk; and/or increased amount of C18:1 in milk; and/or increased amount of C14:1 cis-9 in milk; and/or increased amount of one or more fatty acids selected from the group consisting of C6:0, C8:0, C10:0, C12:0 and C14:0 in milk; and/or optionally any combination thereof.

A female individual having decreased amount of one or more fatty acids in the milk has a higher probability of decreased amount of the one or more fatty acids in the milk than a random female individual (under the same conditions) with whom it is comparable. Two female individuals are comparable if they are, with regards to all discriminating factors except the genotype at the polymorphic site which is used for predicting decreased amount of one or more fatty acids in the milk, random representatives of one and the same population of cattle.

A female individual having increased amount of one or more fatty acids in the milk has a higher probability of increased amount of the one or more fatty acids in the milk than a random female individual (under the same conditions) with whom it is comparable. Two female individuals are comparable if they are, with regards to all discriminating factors except the genotype at the polymorphic site which is used for predicting increased amount of one or more fatty acids in the milk, random representatives of one and the same population of cattle.

Nearly all mammals, including non-human mammals such as cattle and in particular Norwegian Red cattle, are diploid organisms and thus possess at least one copy of the polymorphisms of the invention. In a preferred embodiment, the selected cattle is homozygote with respect to the at least one allele. In another embodiment according to the present invention, the selected cattle is heterozygote with respect to the at least one allele.

The at least one allele, which in a female milk-producing cattle is indicative of a desired milk fatty acid composition, may affect the fatty acid composition in female milk-producing cattle through a number of different mechanisms. The milk fatty acid composition may e.g. be the result of:
a change in a regulatory sequence of a gene which e.g. may affect the level of transcription and/or translation; and/or
a change in amino acid sequence of a protein which e.g. may affect the activity of an enzyme.

Since the at least one allele, which in a female milk-producing cattle is indicative of a desired milk fatty acid composition, may affect the fatty acid composition in female milk-producing cattle through a number of different mechanisms, it is to be understood that presence of the at least one allele may be determined e.g. by a) identifying a change in DNA sequence, b) identifying a change in RNA sequence, such as mRNA sequence, c) identifying a change in protein sequence, d) identifying a change in transcription level, e) identifying a change in expression level and/or f) identifying a change in protein activity, such as enzymatic activity in case the protein in question is an enzyme.

Numerous techniques are known in the art for a) identifying a change in a DNA sequence, b) identifying a change in a RNA sequence, such as a mRNA sequence, c) identifying a change in protein sequence, d) identifying a change in transcription level, e) identifying a change in expression level, and/or f) identifying a change in protein activity, and a person skilled in the art will easily know how to identify such changes.

According to other particular embodiments, the at least one allele is a “fat allele” or a “non-fat allele”, preferably the latter, for C16:0 of at least one polymorphism selected from the group consisting of P #33, P #241-P #248, P #303-P #312, P #314-P #344, P #346-P #475 and, P #477-P #481.

According to other particular embodiments, the at least one allele is a “non-fat allele” or a “fat allele”, preferably the latter, for C18:1 of at least one polymorphism selected from the group consisting of P #19, P #34, P #38, P #39, P #141, P #148, P #153, P #233-P #240, P #242-P #246, P #271-P #278, P #280-P #283, P #285, P #289-P #302, P #311-P #334, P #339, P #340, P #343-P #475, P #477-P #481, P #484-P #494, P #497, P #543, P #588, P #589, P #916, P #911, P #14, P #37, P #903 and P #7.

According to other particular embodiments, the at least one allele is a “non-fat allele” or a “fat allele”, preferably the latter, for C6:0 of at least one polymorphism selected from the group consisting of P #1, P #12, P #15-P #17, P #24, P #41, P #63, P #64, P #69-P #71, P #74-P #79, P #83, P #85-P #87, P #89-P #93, P #97-P #103, P #112-P #118, P #120, P #121, P #123-P #135, P #138-P #141, P #148, P #153-P #168, P #218, P #220-P #226, P #249, P #250, P #253-P #260, P #267, P #495, P #499, P #501, P #503-P #514, P #517-P #539, P #541, P #542, P #544-P #547, P #554-P #585, P #590-P #592, P #595-P #597, P #599-P #614, P #617-P #621, P #623-P #652, P #654-P #669, P #672-P #674, P #676-P #678, P #681, P #682, P #688-P #744, P #746-P #769, P #771-P #813, P #815, P #818-P #824, P #826-P #831 and P #872.

According to other particular embodiments, the at least one allele is a “non-fat allele” or a “fat allele”, preferably the latter, for C8:0 of at least one polymorphism selected from the group consisting of P #1, P #10-P #12, P #16-P #19, P #40-P #45, P #48, P #49, P #51, P #56-P #60, P #63-P #66, P #68-P #93, P #97, P #99-P #101, P #103-P #108, P #111-P #118, P #120, P #121, P #123-P #218, P #220, P #224-P #231, P #254, P #258, P #259, P #268, P #270, P #285-P #288, P #302, P #495, P #496, P #498-P #539, P #541-P #551, P #554-P #586, P #590-P #594, P #628, P #682 and P #683.

According to other particular embodiments, the at least one allele is a “non-fat allele” or a “fat allele”, preferably the latter, for C10:0 of at least one polymorphism selected from the group consisting of P #3, P #8-P #10, P #16-P #20, P #49-P #51, P #56-P #62, P #64, P #69-P #72, P #74, P #76-P #78, P #80-P #96, P #104-P #109, P #111-P #115, P #117, P #118, P #120, P #121, P #127, P #128, P #132, P #133, P #136-P #142, P #148-P #153, P #156-P #166, P #224-P #226, P #285-P #288, P #297-P #302, P #490-P #494, P #501-P #514, P #517-P #539, P #541-P #547, P #551, P #554-P #580, P #585, P #586, P #589-P #593, P #628, P #889-P #891, P #899, P #900, P #902-P #916.

According to other particular embodiments, the at least one allele is a “non-fat allele” or a “fat allele”, preferably the latter, for C12:0 of at least one polymorphism selected from the group consisting of P #3, P #9, P #10, P #16-P #20, P #49-P #53, P #61, P #62, P #64, P #67, P #69-P #72, P #74, P #83, P #85-P #87, P #89-P #96, P #104-P #113, P #115, P #117-P #122, P #127, P #128, P #132, P #133, P #138, P #139, P #141, P #142, P #148-P #151, P #153, P #219, P #224-P #226, P #276, P #285-P #288, P #290-P #302, P #487-P #495, P #497, P #501-P #515, P #517-P #547, P #551, P #554-P #580, P #585, P #586, P #588-P #594, P #887-P #891, P #893-P #916.

According to other particular embodiments, the at least one allele is a “non-fat allele” or a “fat allele”, preferably the latter, for C14:0 of at least one polymorphism selected from the group consisting of P #9, P #10, P #17-P #19, P #34, P #69, P #70, P #83, P #85, P #117, P #120, P #121, P #127, P #128, P #132, P #133, P #139, P #141, P #148-P #151, P #153, P #224-P #226, P #275, P #276, P #279-P #281, P #284-P #302, P #483-P #494, P #497, P #501-P #514, P #517-P #539, P #541, P #543-P #580, P #584-P #594, P #889-P #892, P #899, P #900, P #902-P #916.

According to other particular embodiments, the at least one allele is a “non-fat allele” or a “fat allele”, preferably the latter, for C14:1 cis-9 of at least one polymorphism selected from the group consisting of P #243-P #246, P #314-P #332, P #334-P #340, P #346-P #352, P #354-P #363, P #366-P #392, P #394-P #396, P #398-P #410, P #412-P #475 and P #477-P #481.

It is to be understood that a “fat-allele” for a specific trait is indicative of increased amount of that trait in milk; and a “non-fat allele” for a specific trait is indicative of decreased amount of that trait in milk (“fat allele”, “non-fat allele” and the respective traits are specified in table 1).

According to other particular embodiments, the present invention provides a method for selecting a cattle which possesses a genotype which in a female milk-producing cattle is indicative of a desired milk fatty acid composition, the method comprising:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 1 to 916, such as SEQ ID NOs: 1 to 310; or at a position corresponding to position 60 of a nucleotide sequence which is derived from any one of SEQ ID NOs: 1 to 916, such as SEQ ID NOs: 1 to 310, by 1 to 30, such as 1 to 20, 1 to 15, 1 to 10, 1 to 5, 1 to 3 or 1 to 2 nucleotide substitutions and/or deletions (preferably substitutions); and

selecting said cattle when the one or more nucleotides of the at least one allele is one or more nucleotides corresponding to the allele which in a female milk-producing cattle is indicative of a desired milk fatty acid composition.

In one embodiment according to the present invention, the nucleotide substitution(s) and/or nucleotide deletions (preferably substitutions) are not in the polymorphic site (i.e. position 60) of the derived sequence.

If e.g. the polymorphism is located within the genome of the cattle at a position corresponding to position 60 of the nucleotide sequence set forth in SEQ ID NOs: 1, the “respective polymorphism” referred to above is P #1. If e.g. the polymorphism is located within the genome of the cattle at a position corresponding to position 60 of the nucleotide sequence set forth in SEQ ID NOs: 10, the “respective polymorphism” referred to above is P #10. Similarly, if e.g. the polymorphism is located within the genome of the cattle at a position corresponding to position 60 of a nucleotide sequence which is derived from the nucleotide sequence set forth in SEQ ID NOs: 1 by 1 to 5 nucleotide substitutions, the “respective polymorphism” referred to above is still P #1. Similarly, if e.g. the polymorphism is located within the genome of the cattle at a position corresponding to position 60 of a nucleotide sequence which is derived from the nucleotide sequence set forth in SEQ ID NOs: 10 by 1 to 5 nucleotide substitutions, the “respective polymorphism” referred to above is still P #10.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQIDNOs 33, 241-248, 303-312, 314-344, 346-475 and, 477-481 (C16:0); or at a position corresponding to position 60 of a nucleotide sequence which is derived from any one of 33, 241-248, 303-312, 314-344, 346-475 and, 477-481 (C16:0) by 1 to 30, such as 1 to 20, 1 to 15, 1 to 10, 1 to 5, 1 to 3 or 1 to 2 nucleotide substitutions and/or deletions (preferably substitutions); and

selecting said cattle when the one or more nucleotides of the at least one allele is one or more nucleotide corresponding to the allele which in a female milk-producing cattle is indicative of a desired milk fatty acid composition. In one embodiment, a desired milk fatty acid composition is decreased content of C16:0 in milk and the at least one allele is a “non-fat allele” for C16:0.

In one embodiment according to the present invention, the nucleotide substitution(s) and/or nucleotide deletions (preferably substitutions) are not in the polymorphic site (i.e. position 60) of the derived sequence.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 19, 34, 38, 39, 141, 148, 153, 233-240, 242-246, 271-278, 280-283, 285, 289-302, 311-334, 339, 340, 343-475, 477-481, 484-494, 497, 543, 588, 589, 916, 911, 14, 37, 903 and 7 (C18:1); or at a position corresponding to position 60 of a nucleotide sequence which is derived from any one of SEQ ID NOs: 19, 34, 38, 39, 141, 148, 153, 233-240, 242-246, 271-278, 280-283, 285, 289-302, 311-334, 339, 340, 343-475, 477-481, 484-494, 497, 543, 588, 589, 916, 911, 14, 37, 903 and 7 (C18:1) by 1 to 30, such as 1 to 20, 1 to 15, 1 to 10, 1 to 5, 1 to 3 or 1 to 2 nucleotide substitutions and/or deletions (preferably substitutions); and

selecting said cattle when the one or more nucleotides of the at least one allele is one or more nucleotides corresponding to the allele which in a female milk-producing cattle is indicative of a desired milk fatty acid composition. In one embodiment, a desired milk fatty acid composition is increased content of C18:1 in milk and the at least one allele is a “fat allele” for C18:1.

In one embodiment according to the present invention, the nucleotide substitution(s) and/or nucleotide deletions (preferably substitutions) are not in the polymorphic site (i.e. position 60) of the derived sequence.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 243-246, 314-332, 334-340, 346-352, 354-363, 366-392, 394-396, 398-410, 412-475 and 477-481 (C14:1 cis-9); or at a position corresponding to position 60 of a nucleotide sequence which is derived from any one of SEQ ID NOs: 243-246, 314-332, 334-340, 346-352, 354-363, 366-392, 394-396, 398-410, 412-475 and 477-481 (C14:1 cis-9) by 1 to 30, such as 1 to 20, 1 to 15, 1 to 10, 1 to 5, 1 to 3 or 1 to 2 nucleotide substitutions and/or deletions (preferably substitutions); and

selecting said cattle when the one or more nucleotides of the at least one allele is one or more nucleotides corresponding to the allele which in a female milk-producing cattle is indicative of a desired milk fatty acid composition. In one embodiment, a desired milk fatty acid composition is increased content of C14:1 cis-9 in milk and the at least one allele is a “fat allele” for C14:1 cis-9.

In one embodiment according to the present invention, the nucleotide substitution(s) and/or nucleotide deletions (preferably substitutions) are not in the polymorphic site (i.e. position 60) of the derived sequence.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 1, 12, 15-17, 24, 41, 63, 64, 69-71, 74-79, 83, 85-87, 89-93, 97-103, 112-118, 120, 121, 123-135, 138-141, 148, 153-168, 218, 220-226, 249, 250, 253-260, 267, 495, 499, 501, 503-514, 517-539, 541, 542, 544-547, 554-585, 590-592, 595-597, 599-614, 617-621, 623-652, 654-669, 672-674, 676-678, 681, 682, 688-744, 746-769, 771-813, 815, 818-824, 826-831 and 872 (C6:0); or at a position corresponding to position 60 of a nucleotide sequence which is derived from any one of SEQ ID NOs: 1, 12, 15-17, 24, 41, 63, 64, 69-71, 74-79, 83, 85-87, 89-93, 97-103, 112-118, 120, 121, 123-135, 138-141, 148, 153-168, 218, 220-226, 249, 250, 253-260, 267, 495, 499, 501, 503-514, 517-539, 541, 542, 544-547, 554-585, 590-592, 595-597, 599-614, 617-621, 623-652, 654-669, 672-674, 676-678, 681, 682, 688-744, 746-769, 771-813, 815, 818-824, 826-831 and 872 (C6:0) by 1 to 30, such as 1 to 20, 1 to 15, 1 to 10, 1 to 5, 1 to 3 or 1 to 2 nucleotide substitutions and/or deletions (preferably substitutions); and

selecting said cattle when the one or more nucleotides of the at least one allele is one or more nucleotides corresponding to the allele which in a female milk-producing cattle is indicative of a desired milk fatty acid composition. In one embodiment, a desired milk fatty acid composition is increased content of C6:0 in milk and the at least one allele is a “fat allele” for C6:0.

In one embodiment according to the present invention, the nucleotide substitution(s) and/or nucleotide deletions (preferably substitutions) are not in the polymorphic site (i.e. position 60) of the derived sequence.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 1, 10-12, 16-19, 40-45, 48, 49, 51, 56-60, 63-66, 68-93, 97, 99-101, 103-108, 111-118, 120, 121, 123-218, 220, 224-231, 254, 258, 259, 268, 270, 285-288, 302, 495, 496, 498-539, 541-551, 554-586, 590-594, 628, 682 and 683 (C8:0); or at a position corresponding to position 60 of a nucleotide sequence which is derived from any one of SEQ ID NOs: 1, 10-12, 16-19, 40-45, 48, 49, 51, 56-60, 63-66, 68-93, 97, 99-101, 103-108, 111-118, 120, 121, 123-218, 220, 224-231, 254, 258, 259, 268, 270, 285-288, 302, 495, 496, 498-539, 541-551, 554-586, 590-594, 628, 682 and 683 (C8:0) by 1 to 30, such as 1 to 20, 1 to 15, 1 to 10, 1 to 5, 1 to 3 or 1 to 2 nucleotide substitutions and/or deletions (preferably substitutions); and

selecting said cattle when the one or more nucleotides of the at least one allele is one or more nucleotides corresponding to the allele which in a female milk-producing cattle is indicative of a desired milk fatty acid composition. In one embodiment, a desired milk fatty acid composition is increased content of C8:0 in milk and the at least one allele is a “fat allele” for C8:0.

In one embodiment according to the present invention, the nucleotide substitution(s) and/or nucleotide deletions (preferably substitutions) are not in the polymorphic site (i.e. position 60) of the derived sequence.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 3, 8-10, 16-20, 49-51, 56-62, 64, 69-72, 74, 76-78, 80-96, 104-109, 111-115, 117, 118, 120, 121, 127, 128, 132, 133, 136-142, 148-153, 156-166, 224-226, 285-288, 297-302, 490-494, 501-514, 517-539, 541-547, 551, 554-580, 585, 586, 589-593, 628, 889-891, 899, 900, 902-916 (C10:0); or at a position corresponding to position 60 of a nucleotide sequence which is derived from any one of SEQ ID NOs: 3, 8-10, 16-20, 49-51, 56-62, 64, 69-72, 74, 76-78, 80-96, 104-109, 111-115, 117, 118, 120, 121, 127, 128, 132, 133, 136-142, 148-153, 156-166, 224-226, 285-288, 297-302, 490-494, 501-514, 517-539, 541-547, 551, 554-580, 585, 586, 589-593, 628, 889-891, 899, 900, 902-916 (C10:0) by 1 to 30, such as 1 to 20, 1 to 15, 1 to 10, 1 to 5, 1 to 3 or 1 to 2 nucleotide substitutions and/or deletions (preferably substitutions); and

selecting said cattle when the one or more nucleotides of the at least one allele is one or more nucleotides corresponding to the allele which in a female milk-producing cattle is indicative of a desired milk fatty acid composition. In one embodiment, a desired milk fatty acid composition is increased content of C10:0 in milk and the at least one allele is a “fat allele” for C10:0.

In one embodiment according to the present invention, the nucleotide substitution(s) and/or nucleotide deletions (preferably substitutions) are not in the polymorphic site (i.e. position 60) of the derived sequence.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 3, 9, 10, 16-20, 49-53, 61, 62, 64, 67, 69-72, 74, 83, 85-87, 89-96, 104-113, 115, 117-122, 127, 128, 132, 133, 138, 139, 141, 142, 148-151, 153, 219, 224-226, 276, 285-288, 290-302, 487-495, 497, 501-515, 517-547, 551, 554-580, 585, 586, 588-594, 887-891, 893-916 (C12:0); or at a position corresponding to position 60 of a nucleotide sequence which is derived from any one of SEQ ID NOs: 3, 9, 10, 16-20, 49-53, 61, 62, 64, 67, 69-72, 74, 83, 85-87, 89-96, 104-113, 115, 117-122, 127, 128, 132, 133, 138, 139, 141, 142, 148-151, 153, 219, 224-226, 276, 285-288, 290-302, 487-495, 497, 501-515, 517-547, 551, 554-580, 585, 586, 588-594, 887-891, 893-916 (C12:0) by 1 to 30, such as 1 to 20, 1 to 15, 1 to 10, 1 to 5, 1 to 3 or 1 to 2 nucleotide substitutions and/or deletions (preferably substitutions); and

selecting said cattle when the one or more nucleotides of the at least one allele is one or more nucleotides corresponding to the allele which in a female milk-producing cattle is indicative of a desired milk fatty acid composition. In one embodiment, a desired milk fatty acid composition is increased content of C12:0 in milk and the at least one allele is a “fat allele” for C12:0.

In one embodiment according to the present invention, the nucleotide substitution(s) and/or nucleotide deletions (preferably substitutions) are not in the polymorphic site (i.e. position 60) of the derived sequence.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 9, 10, 17-19, 34, 69, 70, 83, 85, 117, 120, 121, 127, 128, 132, 133, 139, 141, 148-151, 153, 224-226, 275, 276, 279-281, 284-302, 483-494, 497, 501-514, 517-539, 541, 543-580, 584-594, 889-892, 899, 900, 902-916 (C14:0); or at a position corresponding to position 60 of a nucleotide sequence which is derived from any one of SEQ ID NOs: 9, 10, 17-19, 34, 69, 70, 83, 85, 117, 120, 121, 127, 128, 132, 133, 139, 141, 148-151, 153, 224-226, 275, 276, 279-281, 284-302, 483-494, 497, 501-514, 517-539, 541, 543-580, 584-594, 889-892, 899, 900, 902-916 (C14:0) by 1 to 30, such as 1 to 20, 1 to 15, 1 to 10, 1 to 5, 1 to 3 or 1 to 2 nucleotide substitutions and/or deletions (preferably substitutions); and

selecting said cattle when the one or more nucleotides of the at least one allele is one or more nucleotides corresponding to the allele which in a female milk-producing cattle is indicative of a desired milk fatty acid composition. In one embodiment, a desired milk fatty acid composition is increased content of C14:0 in milk and the at least one allele is a “fat allele” for C14:0.

In one embodiment according to the present invention, the nucleotide substitution(s) and/or nucleotide deletions (preferably substitutions) are not in the polymorphic site (i.e. position 60) of the derived sequence.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being selected from the group consisting of P #1 to P #916; and

selecting said cattle when the one or more nucleotides of the at least one allele is one or more nucleotides corresponding to the allele, which in a female milk-producing cattle is indicative of a desired milk fatty acid composition, of the respective polymorphisms listed in table 1.

If e.g. the at least one polymorphism is P #1, the respective polymorphism listed in table 1 is P #1. Similarly, if e.g. the at least one polymorphism is P #10, the respective polymorphism listed in table 1 is P #10.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being selected from the group consisting of P #33, P #241-P #248, P #303-P #312, P #314-P #344, P #346-P #475 and, P #477-P #481 (C16:0); and

selecting said cattle when the one or more nucleotides of the at least one allele is a nucleotide corresponding to the “non-fat allele” for C16:0 of the respective polymorphisms listed in table 1.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being selected from the group consisting of P #19, P #34, P #38, P #39, P #141, P #148, P #153, P #233-P #240, P #242-P #246, P #271-P #278, P #280-P #283, P #285, P #289-P #302, P #311-P #334, P #339, P #340, P #343-P #475, P #477-P #481, P #484-P #494, P #497, P #543, P #588, P #589, P #916, P #911, P #14, P #37, P #903 and P #7 (C18:1); and selecting said cattle when the one or more nucleotides of the at least one allele is one or more nucleotides corresponding to the “fat allele” for C18:1 of the respective polymorphisms listed in table 1.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being selected from the group consisting of P #243-P #246, P #314-P #332, P #334-P #340, P #346-P #352, P #354-P #363, P #366-P #392, P #394-P #396, P #398-P #410, P #412-P #475 and P #477-P #481 (C14:1 cis-9); and

selecting said cattle when the one or more nucleotides of the at least one allele is one or more nucleotides corresponding to the “fat allele” for C14:1 cis-9 of the respective polymorphisms listed in table 1.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being selected from the group consisting of P #1, P #12, P #15-P #17, P #24, P #41, P #63, P #64, P #69-P #71, P #74-P #79, P #83, P #85-P #87, P #89-P #93, P #97-P #103, P #112-P #118, P #120, P #121, P #123-P #135, P #138-P #141, P #148, P #153-P #168, P #218, P #220-P #226, P #249, P #250, P #253-P #260, P #267, P #495, P #499, P #501, P #503-P #514, P #517-P #539, P #541, P #542, P #544-P #547, P #554-P #585, P #590-P #592, P #595-P #597, P #599-P #614, P #617-P #621, P #623-P #652, P #654-P #669, P #672-P #674, P #676-P #678, P #681, P #682, P #688-P #744, P #746-P #769, P #771-P #813, P #815, P #818-P #824, P #826-P #831 and P #872 (C6:0); and

selecting said cattle when the one or more nucleotides of the at least one allele is one or more nucleotides corresponding to the “fat allele” for C6:0 of the respective polymorphisms listed in table 1.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being selected from the group consisting of P #1, P #10-P #12, P #16-P #19, P #40-P #45, P #48, P #49, P #51, P #56-P #60, P #63-P #66, P #68-P #93, P #97, P #99-P #101, P #103-P #108, P #111-P #118, P #120, P #121, P #123-P #218, P #220, P #224-P #231, P #254, P #258, P #259, P #268, P #270, P #285-P #288, P #302, P #495, P #496, P #498-P #539, P #541-P #551, P #554-P #586, P #590-P #594, P #628, P #682 and P #683 (C8:0); and

selecting said cattle when the one or more nucleotides of the at least one allele is one or more nucleotides corresponding to the “fat allele” for C8:0 of the respective polymorphisms listed in table 1.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being selected from the group consisting of P #3, P #8-P #10, P #16-P #20, P #49-P #51, P #56-P #62, P #64, P #69-P #72, P #74, P #76-P #78, P #80-P #96, P #104-P #109, P #111-P #115, P #117, P #118, P #120, P #121, P #127, P #128, P #132, P #133, P #136-P #142, P #148-P #153, P #156-P #166, P #224-P #226, P #285-P #288, P #297-P #302, P #490-P #494, P #501-P #514, P #517-P #539, P #541-P #547, P #551, P #554-P #580, P #585, P #586, P #589-P #593, P #628, P #889-P #891, P #899, P #900, P #902-P #916 (C10:0); and

selecting said cattle when the one or more nucleotides of the at least one allele is one or more nucleotides corresponding to the “fat allele” for C10:0 of the respective polymorphisms listed in table 1.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being selected from the group consisting of P #3, P #9, P #10, P #16-P #20, P #49-P #53, P #61, P #62, P #64, P #67, P #69-P #72, P #74, P #83, P #85-P #87, P #89-P #96, P #104-P #113, P #115, P #117-P #122, P #127, P #128, P #132, P #133, P #138, P #139, P #141, P #142, P #148-P #151, P #153, P #219, P #224-P #226, P #276, P #285-P #288, P #290-P #302, P #487-P #495, P #497, P #501-P #515, P #517-P #547, P #551, P #554-P #580, P #585, P #586, P #588-P #594, P #887-P #891, P #893-P #916 (C12:0); and

selecting said cattle when the one or more nucleotides of the at least one allele is one or more nucleotides corresponding to the “fat allele” for C12:0 of the respective polymorphisms listed in table 1.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being selected from the group consisting of P #9, P #10, P #17-P #19, P #34, P #69, P #70, P #83, P #85, P #117, P #120, P #121, P #127, P #128, P #132, P #133, P #139, P #141, P #148-P #151, P #153, P #224-P #226, P #275, P #276, P #279-P #281, P #284-P #302, P #483-P #494, P #497, P #501-P #514, P #517-P #539, P #541, P #543-P #580, P #584-P #594, P #889-P #892, P #899, P #900, P #902-P #916 (C14:0); and

selecting said cattle when the one or more nucleotides of the at least one allele is one or more nucleotides corresponding to the “fat allele” for C14:0 of the respective polymorphisms listed in table 1.

Numerous techniques are known in the art for determining the identity of one or more nucleotides of an allele present at a polymorphic site. For example, the determination may involve sequence analysis of the cattle to be tested using, e.g., traditional sequence methodologies (e.g., the “dideoxy-mediated chain termination method,” also known as the “Sanger Method” (Sanger, F., et al., J. Molec. Biol. 94: 441 (1975); Prober et al. Science 238: 336-340 (1987)) and the “chemical degradation method” also known as the “Maxam-Gilbert method” (Maxam, A. M., et al., Proc. Natl. Acad. Sci. (U.S.A) 74: 560 (1977). Alternatively, the determination may involve single base extension of DNA oligonucleotides terminating at the polymorphic site (e.g. iPLEX assays from Sequenom (San Diego, USA) and Infinium assays from Illumina (San Diego, USA), allele-specific ligation assays (e.g. Axiom technology from Affymetrix (San Diego, USA), allele-specific PCR (e.g. SNPtype assays from Fluidigm (San Francisco) or KASP assays from LGC Genomics (Teddington, UK)), or competitive hybridisation of probes complementary to the different alleles (e.g. the TaqMan assay from Applied Biosystems (Foster City, USA)).

Methods for the detection of allelic variation are also reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2nd Edition by Newton & Graham, BIOS Scientific Publishers Limited, 1997.

For analyzing SNPs, it may for example be appropriate to use oligonucleotides specific for alternative SNP alleles. Such oligonucleotides which detect single nucleotide variations in target sequences may be referred to by such terms as “allele-specific oligonucleotides”, “allele-specific probes”, or “allele-specific primers”. The design and use of allele-specific probes for analyzing polymorphisms is described in, e.g., Mutation Detection A Practical Approach, ed. Cotton et al. Oxford University Press, 1998; Saiki et al., Nature 324, 163-166 (1986); Dattagupta, EP235726; and Saiki, WO 89/11548.

Non-Human Gamete

The present invention provides in a second aspect a non-human gamete, such as an isolated non-human gamete, comprising within its genome at least one allele which in a female milk-producing cattle is indicative of a desired milk fatty acid composition.

As used herein, “isolated” means that an organism or a biological component, such as a cell, population of cells or a nucleic acid molecule, has been separated from its natural environment.

The at least one allele referred to in the second aspect of the present invention refers to an allele which determines a specific phenotype (milk fatty acid composition) only in female milk-producing cattle. A non-human gamete which comprises within its genome at least one allele which in a female milk-producing cattle is indicative of a desired milk fatty acid composition may find use in various breeding programs.

Since the at least one allele referred to in the second aspect of the present invention determines a specific phenotype only in female milk-producing cattle, it has been decided to differentiate between a female cattle, which not necessarily produces milk, and a female milk-producing cattle, which produces milk by definition.

According to particular embodiments, the non-human gamete is a non-human semen. In particular embodiments the non-human semen has been isolated from a male cattle, in particular from a male cattle selected by the method according to the first aspect of the present invention.

Semen, also known as seminal fluid, is an organic fluid that may contain spermatozoa. It is secreted by the gonads (sexual glands) and other sexual organs of male or hermaphroditic animals and can fertilize female ova. In one embodiment according to the present invention said non-human semen comprises at least one spermatozoa.

According to particular embodiments, the non-human gamete is a non-human spermatozoa. In particular embodiments, the non-human spermatozoa has been isolated from a male cattle, in particular from a male cattle selected by the method according to the first aspect of the present invention.

A spermatozoon is a motile sperm cell, or moving form of the haploid cell that is the male gamete. A spermatozoon joins an ovum to form a zygote. A zygote is a single cell, with a complete set of chromosomes, that normally develops into an embryo.

According to particular embodiments, the non-human gamete is a non-human sperm. In particular embodiments, the non-human sperm has been produced by a male cattle, in particular a male cattle selected by the method according to the first aspect of the present invention.

Sperm is the male reproductive cell. In the types of sexual reproduction known as anisogamy and its subtype oogamy, there is a marked difference in the size of the gametes with the smaller one being termed the “male” or sperm. A uniflagellar sperm that is motile is referred to as a spermatozoon, whereas a non-motile sperm cell is referred to as a spermatium. Sperm cannot divide and have a limited life span, but after fusion with egg cells during fertilization, a new organism begins developing, starting as a totipotent zygote.

In one embodiment according to the present invention the non-human sperm is a non-human spermatozoon. In another embodiment according to the present invention, the non-human sperm is a non-human spermatium.

According to particular embodiments, the non-human gamete is a non-human ovum. In another embodiment according to the present invention, the non-human ovum is fertilized. In another embodiment according to the present invention, the non-human ovum is unfertilized. In particular embodiments, the non-human ovum has been isolated from a female milk-producing cattle, in particular from a female cattle, preferably a female milk-producing cattle, selected by the method according to the first aspect of the present invention.

The egg cell, or ovum, is the female reproductive cell (gamete) in oogamous organisms. The egg cell is typically not capable of active movement, and it is much larger (visible to the naked eye) than the motile sperm cells. When egg and sperm fuse, a diploid cell (the zygote) is formed, which gradually grows into a new organism.

In one embodiment according to the present invention, said non-human gamete is selected from the group consisting of bovine gamete, cattle gamete and in particular Norwegian Red cattle gamete.

In one embodiment according to the present invention, said female milk-producing cattle is selected from the group consisting of female milk-producing cattle and female milk-producing Norwegian Red cattle.

According to particular embodiments, the at least one allele is an allele of at least one polymorphism. The at least one polymorphism may be selected from the polymorphisms listed in table 1.

In another embodiment according to the second aspect of the present invention, the at least one allele is an allele of at least one polymorphism selected from the group consisting of P #1 to P #916. In another embodiment according to the second aspect of the present invention, the at least one allele is an allele of at least one polymorphism selected from the group consisting of P #33, P #241-P #248, P #303-P #312, P #314-P #344, P #346-P #475 and, P #477-P #481 (C16:0), preferably the at least one allele represents a “non-fat allele” for C16:0. In another embodiment according to the second aspect of the present invention, the at least one allele is an allele of at least one polymorphism selected from the group consisting of P #19, P #34, P #38, P #39, P #141, P #148, P #153, P #233-P #240, P #242-P #246, P #271-P #278, P #280-P #283, P #285, P #289-P #302, P #311-P #334, P #339, P #340, P #343-P #475, P #477-P #481, P #484-P #494, P #497, P #543, P #588, P #589, P #916, P #911, P #14, P #37, P #903 and P #7 (C18:1), preferably the at least one allele represents a “fat allele” for C18:1. In another embodiment according to the second aspect of the present invention, the at least one allele is an allele of at least one polymorphism selected from the group consisting of P #1, P #12, P #15-P #17, P #24, P #41, P #63, P #64, P #69-P #71, P #74-P #79, P #83, P #85-P #87, P #89-P #93, P #97-P #103, P #112-P #118, P #120, P #121, P #123-P #135, P #138-P #141, P #148, P #153-P #168, P #218, P #220-P #226, P #249, P #250, P #253-P #260, P #267, P #495, P #499, P #501, P #503-P #514, P #517-P #539, P #541, P #542, P #544-P #547, P #554-P #585, P #590-P #592, P #595-P #597, P #599-P #614, P #617-P #621, P #623-P #652, P #654-P #669, P #672-P #674, P #676-P #678, P #681, P #682, P #688-P #744, P #746-P #769, P #771-P #813, P #815, P #818-P #824, P #826-P #831 and P #872 (C6:0), preferably the at least one allele represents a “fat allele” for C6:0. In another embodiment according to the second aspect of the present invention, the at least one allele is an allele of at least one polymorphism selected from the group consisting of P #1, P #10-P #12, P #16-P #19, P #40-P #45, P #48, P #49, P #51, P #56-P #60, P #63-P #66, P #68-P #93, P #97, P #99-P #101, P #103-P #108, P #111-P #118, P #120, P #121, P #123-P #218, P #220, P #224-P #231, P #254, P #258, P #259, P #268, P #270, P #285-P #288, P #302, P #495, P #496, P #498-P #539, P #541-P #551, P #554-P #586, P #590-P #594, P #628, P #682 and P #683 (C8:0), preferably the at least one allele represents a “fat allele” for C8:0. In another embodiment according to the second aspect of the present invention, the at least one allele is an allele of at least one polymorphism selected from the group consisting of P #3, P #8-P #10, P #16-P #20, P #49-P #51, P #56-P #62, P #64, P #69-P #72, P #74, P #76-P #78, P #80-P #96, P #104-P #109, P #111-P #115, P #117, P #118, P #120, P #121, P #127, P #128, P #132, P #133, P #136-P #142, P #148-P #153, P #156-P #166, P #224-P #226, P #285-P #288, P #297-P #302, P #490-P #494, P #501-P #514, P #517-P #539, P #541-P #547, P #551, P #554-P #580, P #585, P #586, P #589-P #593, P #628, P #889-P #891, P #899, P #900, P #902-P #916 (C10:0), preferably the at least one allele represents a “fat allele” for C10:0. In another embodiment according to the second aspect of the present invention, the at least one allele is an allele of at least one polymorphism selected from the group consisting of P #3, P #9, P #10, P #16-P #20, P #49-P #53, P #61, P #62, P #64, P #67, P #69-P #72, P #74, P #83, P #85-P #87, P #89-P #96, P #104-P #113, P #115, P #117-P #122, P #127, P #128, P #132, P #133, P #138, P #139, P #141, P #142, P #148-P #151, P #153, P #219, P #224-P #226, P #276, P #285-P #288, P #290-P #302, P #487-P #495, P #497, P #501-P #515, P #517-P #547, P #551, P #554-P #580, P #585, P #586, P #588-P #594, P #887-P #891, P #893-P #916 (C12:0), preferably the at least one allele represents a “fat allele” for C12:0. In another embodiment according to the second aspect of the present invention, the at least one allele is an allele of at least one polymorphism selected from the group consisting of P #9, P #10, P #17-P #19, P #34, P #69, P #70, P #83, P #85, P #117, P #120, P #121, P #127, P #128, P #132, P #133, P #139, P #141, P #148-P #151, P #153, P #224-P #226, P #275, P #276, P #279-P #281, P #284-P #302, P #483-P #494, P #497, P #501-P #514, P #517-P #539, P #541, P #543-P #580, P #584-P #594, P #889-P #892, P #899, P #900, P #902-P #916 (C14:0), preferably the at least one allele represents a “fat allele” for C14:0. In another embodiment according to the second aspect of the present invention, the at least one allele is an allele of at least one polymorphism selected from the group consisting of P #243-P #246, P #314-P #332, P #334-P #340, P #346-P #352, P #354-P #363, P #366-P #392, P #394-P #396, P #398-P #410, P #412-P #475 and P #477-P #481 (C14:1 cis-9), preferably the at least one allele represents a “fat allele” for C14:1 cis-9.

In another embodiment according to the second aspect of the present invention, a desired milk fatty acid composition is decreased amount of C16:0 in milk; and/or increased amount of C18:1 in milk; and/or increased amount of C14:1 cis-9 in milk; and/or increased amount of one or more fatty acids selected from the group consisting of C6:0, C8:0, C10:0, C12:0 and C14:0 in milk; and/or optionally any combination thereof.

According to other particular embodiments, the at least one allele is a “fat allele” or a “non-fat allele”, preferably the latter, for C16:0 of at least one polymorphism selected from the group consisting of P #33, P #241-P #248, P #303-P #312, P #314-P #344, P #346-P #475 and, P #477-P #481.

According to other particular embodiments, the at least one allele is a “non-fat allele” or a “fat allele”, preferably the latter, for C18:1 of at least one polymorphism selected from the group consisting of P #19, P #34, P #38, P #39, P #141, P #148, P #153, P #233-P #240, P #242-P #246, P #271-P #278, P #280-P #283, P #285, P #289-P #302, P #311-P #334, P #339, P #340, P #343-P #475, P #477-P #481, P #484-P #494, P #497, P #543, P #588, P #589, P #916, P #911, P #14, P #37, P #903 and P #7.

According to other particular embodiments, the at least one allele is a “non-fat allele” or a “fat allele”, preferably the latter, for C6:0 of at least one polymorphism selected from the group consisting of P #1, P #12, P #15-P #17, P #24, P #41, P #63, P #64, P #69-P #71, P #74-P #79, P #83, P #85-P #87, P #89-P #93, P #97-P #103, P #112-P #118, P #120, P #121, P #123-P #135, P #138-P #141, P #148, P #153-P #168, P #218, P #220-P #226, P #249, P #250, P #253-P #260, P #267, P #495, P #499, P #501, P #503-P #514, P #517-P #539, P #541, P #542, P #544-P #547, P #554-P #585, P #590-P #592, P #595-P #597, P #599-P #614, P #617-P #621, P #623-P #652, P #654-P #669, P #672-P #674, P #676-P #678, P #681, P #682, P #688-P #744, P #746-P #769, P #771-P #813, P #815, P #818-P #824, P #826-P #831 and P #872.

According to other particular embodiments, the at least one allele is a “non-fat allele” or a “fat allele”, preferably the latter, for C8:0 of at least one polymorphism selected from the group consisting of P #1, P #10-P #12, P #16-P #19, P #40-P #45, P #48, P #49, P #51, P #56-P #60, P #63-P #66, P #68-P #93, P #97, P #99-P #101, P #103-P #108, P #111-P #118, P #120, P #121, P #123-P #218, P #220, P #224-P #231, P #254, P #258, P #259, P #268, P #270, P #285-P #288, P #302, P #495, P #496, P #498-P #539, P #541-P #551, P #554-P #586, P #590-P #594, P #628, P #682 and P #683.

According to other particular embodiments, the at least one allele is a “non-fat allele” or a “fat allele”, preferably the latter, for C10:0 of at least one polymorphism selected from the group consisting of P #3, P #8-P #10, P #16-P #20, P #49-P #51, P #56-P #62, P #64, P #69-P #72, P #74, P #76-P #78, P #80-P #96, P #104-P #109, P #111-P #115, P #117, P #118, P #120, P #121, P #127, P #128, P #132, P #133, P #136-P #142, P #148-P #153, P #156-P #166, P #224-P #226, P #285-P #288, P #297-P #302, P #490-P #494, P #501-P #514, P #517-P #539, P #541-P #547, P #551, P #554-P #580, P #585, P #586, P #589-P #593, P #628, P #889-P #891, P #899, P #900, P #902-P #916.

According to other particular embodiments, the at least one allele is a “non-fat allele” or a “fat allele”, preferably the latter, for C12:0 of at least one polymorphism selected from the group consisting of P #3, P #9, P #10, P #16-P #20, P #49-P #53, P #61, P #62, P #64, P #67, P #69-P #72, P #74, P #83, P #85-P #87, P #89-P #96, P #104-P #113, P #115, P #117-P #122, P #127, P #128, P #132, P #133, P #138, P #139, P #141, P #142, P #148-P #151, P #153, P #219, P #224-P #226, P #276, P #285-P #288, P #290-P #302, P #487-P #495, P #497, P #501-P #515, P #517-P #547, P #551, P #554-P #580, P #585, P #586, P #588-P #594, P #887-P #891, P #893-P #916.

According to other particular embodiments, the at least one allele is a “non-fat allele” or a “fat allele”, preferably the latter, for C14:0 of at least one polymorphism selected from the group consisting of P #9, P #10, P #17-P #19, P #34, P #69, P #70, P #83, P #85, P #117, P #120, P #121, P #127, P #128, P #132, P #133, P #139, P #141, P #148-P #151, P #153, P #224-P #226, P #275, P #276, P #279-P #281, P #284-P #302, P #483-P #494, P #497, P #501-P #514, P #517-P #539, P #541, P #543-P #580, P #584-P #594, P #889-P #892, P #899, P #900, P #902-P #916.

According to other particular embodiments, the at least one allele is a “non-fat allele” or a “fat allele”, preferably the latter, for C14:1 cis-9 of at least one polymorphism selected from the group consisting of P #243-P #246, P #314-P #332, P #334-P #340, P #346-P #352, P #354-P #363, P #366-P #392, P #394-P #396, P #398-P #410, P #412-P #475 and P #477-P #481.

It is to be understood that a “fat-allele” for a specific trait is indicative of increased amount of that trait in milk; and a “non-fat allele” for a specific trait is indicative of decreased amount of that trait in milk (“fat allele”, “non-fat allele” and the respective traits are specified in table 1).

According to particular embodiments, the non-human gamete comprises within its genome at least one nucleotide sequence selected from the group consisting of a) the nucleotide sequences set forth in any one of SEQ ID NOs: 1 to 916; and b) nucleotide sequences which are derived from any one of SEQ ID NOs: 1 to 916 by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition.

In one embodiment according to the present invention, the nucleotide substitution(s) and/or nucleotide deletions (preferably substitutions) are not in the polymorphic site (i.e. position 60) of the derived sequence.

According to particular embodiments, the non-human gamete comprises within its genome at least one nucleotide sequence selected from the group consisting of a) the nucleotide sequences set forth in any one of SEQIDNOs 33, 241-248, 303-312, 314-344, 346-475 and, 477-481 (C16:0); and b) nucleotide sequences which are derived from any one of SEQIDNOs 33, 241-248, 303-312, 314-344, 346-475 and, 477-481 (C16:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “non-fat allele” for C16:0.

According to particular embodiments, the non-human gamete comprises within its genome at least one nucleotide sequence selected from the group consisting of a) the nucleotide sequences set forth in any one of SEQ ID NOs: 19, 34, 38, 39, 141, 148, 153, 233-240, 242-246, 271-278, 280-283, 285, 289-302, 311-334, 339, 340, 343-475, 477-481, 484-494, 497, 543, 588, 589, 916, 911, 14, 37, 903 and 7 (C18:1); and b) nucleotide sequences which are derived from any one of SEQ ID NOs: 19, 34, 38, 39, 141, 148, 153, 233-240, 242-246, 271-278, 280-283, 285, 289-302, 311-334, 339, 340, 343-475, 477-481, 484-494, 497, 543, 588, 589, 916, 911, 14, 37, 903 and 7 (C18:1) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C18:1.

According to particular embodiments, the non-human gamete comprises within its genome at least one nucleotide sequence selected from the group consisting of a) the nucleotide sequences set forth in any one of SEQ ID NOs: 243-246, 314-332, 334-340, 346-352, 354-363, 366-392, 394-396, 398-410, 412-475 and 477-481 (C14:1 cis-9); and b) nucleotide sequences which are derived from any one of SEQ ID NOs: 243-246, 314-332, 334-340, 346-352, 354-363, 366-392, 394-396, 398-410, 412-475 and 477-481 (C14:1 cis-9) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C14:1 cis-9.

According to particular embodiments, the non-human gamete comprises within its genome at least one nucleotide sequence selected from the group consisting of a) the nucleotide sequences set forth in any one of SEQ ID NOs: 1, 12, 15-17, 24, 41, 63, 64, 69-71, 74-79, 83, 85-87, 89-93, 97-103, 112-118, 120, 121, 123-135, 138-141, 148, 153-168, 218, 220-226, 249, 250, 253-260, 267, 495, 499, 501, 503-514, 517-539, 541, 542, 544-547, 554-585, 590-592, 595-597, 599-614, 617-621, 623-652, 654-669, 672-674, 676-678, 681, 682, 688-744, 746-769, 771-813, 815, 818-824, 826-831 and 872 (C6:0); and b) nucleotide sequences which are derived from any one of SEQ ID NOs: 1, 12, 15-17, 24, 41, 63, 64, 69-71, 74-79, 83, 85-87, 89-93, 97-103, 112-118, 120, 121, 123-135, 138-141, 148, 153-168, 218, 220-226, 249, 250, 253-260, 267, 495, 499, 501, 503-514, 517-539, 541, 542, 544-547, 554-585, 590-592, 595-597, 599-614, 617-621, 623-652, 654-669, 672-674, 676-678, 681, 682, 688-744, 746-769, 771-813, 815, 818-824, 826-831 and 872 (C6:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C6:0.

According to particular embodiments, the non-human gamete comprises within its genome at least one nucleotide sequence selected from the group consisting of a) the nucleotide sequences set forth in any one of SEQ ID NOs: 1, 10-12, 16-19, 40-45, 48, 49, 51, 56-60, 63-66, 68-93, 97, 99-101, 103-108, 111-118, 120, 121, 123-218, 220, 224-231, 254, 258, 259, 268, 270, 285-288, 302, 495, 496, 498-539, 541-551, 554-586, 590-594, 628, 682 and 683 (C8:0); and b) nucleotide sequences which are derived from any one of SEQ ID NOs: 1, 10-12, 16-19, 40-45, 48, 49, 51, 56-60, 63-66, 68-93, 97, 99-101, 103-108, 111-118, 120, 121, 123-218, 220, 224-231, 254, 258, 259, 268, 270, 285-288, 302, 495, 496, 498-539, 541-551, 554-586, 590-594, 628, 682 and 683 (C8:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C8:0.

According to particular embodiments, the non-human gamete comprises within its genome at least one nucleotide sequence selected from the group consisting of a) the nucleotide sequences set forth in any one of SEQ ID NOs: 3, 8-10, 16-20, 49-51, 56-62, 64, 69-72, 74, 76-78, 80-96, 104-109, 111-115, 117, 118, 120, 121, 127, 128, 132, 133, 136-142, 148-153, 156-166, 224-226, 285-288, 297-302, 490-494, 501-514, 517-539, 541-547, 551, 554-580, 585, 586, 589-593, 628, 889-891, 899, 900, 902-916 (C10:0); and b) nucleotide sequences which are derived from any one of SEQ ID NOs: 3, 8-10, 16-20, 49-51, 56-62, 64, 69-72, 74, 76-78, 80-96, 104-109, 111-115, 117, 118, 120, 121, 127, 128, 132, 133, 136-142, 148-153, 156-166, 224-226, 285-288, 297-302, 490-494, 501-514, 517-539, 541-547, 551, 554-580, 585, 586, 589-593, 628, 889-891, 899, 900, 902-916 (C10:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C10:0.

According to particular embodiments, the non-human gamete comprises within its genome at least one nucleotide sequence selected from the group consisting of a) the nucleotide sequences set forth in any one of SEQ ID NOs: 3, 9, 10, 16-20, 49-53, 61, 62, 64, 67, 69-72, 74, 83, 85-87, 89-96, 104-113, 115, 117-122, 127, 128, 132, 133, 138, 139, 141, 142, 148-151, 153, 219, 224-226, 276, 285-288, 290-302, 487-495, 497, 501-515, 517-547, 551, 554-580, 585, 586, 588-594, 887-891, 893-916 (C12:0); and b) nucleotide sequences which are derived from any one of SEQ ID NOs: 3, 9, 10, 16-20, 49-53, 61, 62, 64, 67, 69-72, 74, 83, 85-87, 89-96, 104-113, 115, 117-122, 127, 128, 132, 133, 138, 139, 141, 142, 148-151, 153, 219, 224-226, 276, 285-288, 290-302, 487-495, 497, 501-515, 517-547, 551, 554-580, 585, 586, 588-594, 887-891, 893-916 (C12:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C12:0.

According to particular embodiments, the non-human gamete comprises within its genome at least one nucleotide sequence selected from the group consisting of a) the nucleotide sequences set forth in any one of SEQ ID NOs: 9, 10, 17-19, 34, 69, 70, 83, 85, 117, 120, 121, 127, 128, 132, 133, 139, 141, 148-151, 153, 224-226, 275, 276, 279-281, 284-302, 483-494, 497, 501-514, 517-539, 541, 543-580, 584-594, 889-892, 899, 900, 902-916 (C14:0); and b) nucleotide sequences which are derived from any one of SEQ ID NOs: 9, 10, 17-19, 34, 69, 70, 83, 85, 117, 120, 121, 127, 128, 132, 133, 139, 141, 148-151, 153, 224-226, 275, 276, 279-281, 284-302, 483-494, 497, 501-514, 517-539, 541, 543-580, 584-594, 889-892, 899, 900, 902-916 (C14:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C14:0.

Method for Selective Breeding

The present invention provides in a third aspect a method for selective breeding of a cattle, the method comprises:

providing non-human semen or non-human sperm comprising within its genome at least one allele which in a female milk-producing cattle is indicative of a desired milk fatty acid composition; and

fertilizing a (suitable) female (milk-producing) cattle, preferably a female (milk-producing) cattle selected by the method according to the first aspect of the present invention, using the non-human semen or non-human sperm.

The present invention provides in an alternative aspect a method for selective breeding of a cattle, the method comprises:

fertilizing a (suitable) female (milk-producing) cattle, preferably a female (milk-producing) cattle selected by the method according to the first aspect of the present invention, using the semen or sperm according to the second aspect of the present invention.

In the context of the present invention, a suitable female (milk-producing) cattle is a cattle that is capable of being fertilized with the semen or sperm in the sense that the sperm fuses with an ovum and thereby initiates development of a new organism.

The cattle referred to in the above method may be male or female. In one embodiment according to the present invention, said cattle is male. In another embodiment according to the present invention, said cattle is a female or a female milk-producing cattle.

In certain embodiments according to the present invention, said cattle is selected from the group consisting of Norwegian Red cattle.

In one embodiment according to the present invention, said (suitable) female (milk-producing) cattle is selected from the group consisting of female milk-producing Norwegian Red cattle.

According to particular embodiments, the method comprises:

providing at least one non-human semen or non-human sperm as defined in the second aspect of the present invention; and

fertilizing a (suitable) female (milk-producing) cattle, preferably a female (milk-producing) cattle selected by the method according to the first aspect of the present invention, using the at least one non-human semen or non-human sperm.

In one embodiment according to the present invention, the (suitable) female (milk-producing) cattle that is to be fertilized using the semen or sperm according to the second aspect of the present invention comprises within its genome at least one allele which in a female milk-producing cattle is indicative of a desired milk fatty acid composition.

Nearly all mammals, including non-human mammals such as cattle and in particular Norwegian Red cattle, are diploid organisms and thus possess at least one copy of the polymorphisms of the invention. In a preferred embodiment, the (suitable) female (milk-producing) cattle that is to be fertilized using the semen or sperm according to the second aspect of the present invention is homozygote with respect to the at least one allele which in a female milk-producing cattle is indicative of a desired milk fatty acid composition. In an alternative embodiment, the (suitable) female (milk-producing) cattle that is to be fertilized using the semen or sperm according to the second aspect of the present invention is heterozygote with respect to the at least one allele which in a female milk-producing cattle is indicative of a desired milk fatty acid composition.

In one embodiment according to the third aspect of the present invention, the at least one allele is an allele of at least one polymorphism, such as at least one single nucleotide polymorphism (SNP). In a preferred embodiment, the at least one polymorphism is selected from the polymorphisms listed in table 1.

In another embodiment according to the third aspect of the present invention, the at least one allele is an allele of at least one polymorphism selected from the group consisting of P #1 to P #916, such as P #1 to P #310. In another embodiment according to the third aspect of the present invention, the at least one allele is an allele of at least one polymorphism selected from the group consisting of P #33, P #241-P #248, P #303-P #312, P #314-P #344, P #346-P #475 and, P #477-P #481 (C16:0), preferably the at least one allele represents a “non-fat allele” for C16:0. In another embodiment according to the third aspect of the present invention, the at least one allele is an allele of at least one polymorphism selected from the group consisting of P #19, P #34, P #38, P #39, P #141, P #148, P #153, P #233-P #240, P #242-P #246, P #271-P #278, P #280-P #283, P #285, P #289-P #302, P #311-P #334, P #339, P #340, P #343-P #475, P #477-P #481, P #484-P #494, P #497, P #543, P #588, P #589, P #916, P #911, P #14, P #37, P #903 and P #7 (C18:1), preferably the at least one allele represents a “fat allele” for C18:1. In another embodiment according to the third aspect of the present invention, the at least one allele is an allele of at least one polymorphism selected from the group consisting of P #1, P #12, P #15-P #17, P #24, P #41, P #63, P #64, P #69-P #71, P #74-P #79, P #83, P #85-P #87, P #89-P #93, P #97-P #103, P #112-P #118, P #120, P #121, P #123-P #135, P #138-P #141, P #148, P #153-P #168, P #218, P #220-P #226, P #249, P #250, P #253-P #260, P #267, P #495, P #499, P #501, P #503-P #514, P #517-P #539, P #541, P #542, P #544-P #547, P #554-P #585, P #590-P #592, P #595-P #597, P #599-P #614, P #617-P #621, P #623-P #652, P #654-P #669, P #672-P #674, P #676-P #678, P #681, P #682, P #688-P #744, P #746-P #769, P #771-P #813, P #815, P #818-P #824, P #826-P #831 and P #872 (C6:0), preferably the at least one allele represents a “fat allele” for C6:0. In another embodiment according to the third aspect of the present invention, the at least one allele is an allele of at least one polymorphism selected from the group consisting of P #1, P #10-P #12, P #16-P #19, P #40-P #45, P #48, P #49, P #51, P #56-P #60, P #63-P #66, P #68-P #93, P #97, P #99-P #101, P #103-P #108, P #111-P #118, P #120, P #121, P #123-P #218, P #220, P #224-P #231, P #254, P #258, P #259, P #268, P #270, P #285-P #288, P #302, P #495, P #496, P #498-P #539, P #541-P #551, P #554-P #586, P #590-P #594, P #628, P #682 and P #683 (C8:0), preferably the at least one allele represents a “fat allele” for C8:0. In another embodiment according to the third aspect of the present invention, the at least one allele is an allele of at least one polymorphism selected from the group consisting of P #3, P #8-P #10, P #16-P #20, P #49-P #51, P #56-P #62, P #64, P #69-P #72, P #74, P #76-P #78, P #80-P #96, P #104-P #109, P #111-P #115, P #117, P #118, P #120, P #121, P #127, P #128, P #132, P #133, P #136-P #142, P #148-P #153, P #156-P #166, P #224-P #226, P #285-P #288, P #297-P #302, P #490-P #494, P #501-P #514, P #517-P #539, P #541-P #547, P #551, P #554-P #580, P #585, P #586, P #589-P #593, P #628, P #889-P #891, P #899, P #900, P #902-P #916 (C10:0), preferably the at least one allele represents a “fat allele” for C10:0. In another embodiment according to the third aspect of the present invention, the at least one allele is an allele of at least one polymorphism selected from the group consisting of P #3, P #9, P #10, P #16-P #20, P #49-P #53, P #61, P #62, P #64, P #67, P #69-P #72, P #74, P #83, P #85-P #87, P #89-P #96, P #104-P #113, P #115, P #117-P #122, P #127, P #128, P #132, P #133, P #138, P #139, P #141, P #142, P #148-P #151, P #153, P #219, P #224-P #226, P #276, P #285-P #288, P #290-P #302, P #487-P #495, P #497, P #501-P #515, P #517-P #547, P #551, P #554-P #580, P #585, P #586, P #588-P #594, P #887-P #891, P #893-P #916 (C12:0), preferably the at least one allele represents a “fat allele” for C12:0. In another embodiment according to the third aspect of the present invention, the at least one allele is an allele of at least one polymorphism selected from the group consisting of P #9, P #10, P #17-P #19, P #34, P #69, P #70, P #83, P #85, P #117, P #120, P #121, P #127, P #128, P #132, P #133, P #139, P #141, P #148-P #151, P #153, P #224-P #226, P #275, P #276, P #279-P #281, P #284-P #302, P #483-P #494, P #497, P #501-P #514, P #517-P #539, P #541, P #543-P #580, P #584-P #594, P #889-P #892, P #899, P #900, P #902-P #916 (C14:0), preferably the at least one allele represents a “fat allele” for C14:0. In another embodiment according to the third aspect of the present invention, the at least one allele is an allele of at least one polymorphism selected from the group consisting of P #243-P #246, P #314-P #332, P #334-P #340, P #346-P #352, P #354-P #363, P #366-P #392, P #394-P #396, P #398-P #410, P #412-P #475 and P #477-P #481 (C14:1 cis-9), preferably the at least one allele represents a “fat allele” for C14:1 cis-9.

In another embodiment according to the third aspect of the present invention, a desired milk fatty acid composition is decreased amount of C16:0 in milk; and/or increased amount of C18:1 in milk; and/or increased amount of C14:1 cis-9 in milk; and/or increased amount of one or more fatty acids selected from the group consisting of C6:0, C8:0, C10:0, C12:0 and C14:0 in milk; and/or optionally any combination thereof.

According to other particular embodiments, the at least one allele is a “fat allele” or a “non-fat allele”, preferably the latter, for C16:0 of at least one polymorphism selected from the group consisting of P #33, P #241-P #248, P #303-P #312, P #314-P #344, P #346-P #475 and, P #477-P #481.

According to other particular embodiments, the at least one allele is a “non-fat allele” or a “fat allele”, preferably the latter, for C18:1 of at least one polymorphism selected from the group consisting of P #19, P #34, P #38, P #39, P #141, P #148, P #153, P #233-P #240, P #242-P #246, P #271-P #278, P #280-P #283, P #285, P #289-P #302, P #311-P #334, P #339, P #340, P #343-P #475, P #477-P #481, P #484-P #494, P #497, P #543, P #588, P #589, P #916, P #911, P #14, P #37, P #903 and P #7.

According to other particular embodiments, the at least one allele is a “non-fat allele” or a “fat allele”, preferably the latter, for C6:0 of at least one polymorphism selected from the group consisting of P #1, P #12, P #15-P #17, P #24, P #41, P #63, P #64, P #69-P #71, P #74-P #79, P #83, P #85-P #87, P #89-P #93, P #97-P #103, P #112-P #118, P #120, P #121, P #123-P #135, P #138-P #141, P #148, P #153-P #168, P #218, P #220-P #226, P #249, P #250, P #253-P #260, P #267, P #495, P #499, P #501, P #503-P #514, P #517-P #539, P #541, P #542, P #544-P #547, P #554-P #585, P #590-P #592, P #595-P #597, P #599-P #614, P #617-P #621, P #623-P #652, P #654-P #669, P #672-P #674, P #676-P #678, P #681, P #682, P #688-P #744, P #746-P #769, P #771-P #813, P #815, P #818-P #824, P #826-P #831 and P #872.

According to other particular embodiments, the at least one allele is a “non-fat allele” or a “fat allele”, preferably the latter, for C8:0 of at least one polymorphism selected from the group consisting of P #1, P #10-P #12, P #16-P #19, P #40-P #45, P #48, P #49, P #51, P #56-P #60, P #63-P #66, P #68-P #93, P #97, P #99-P #101, P #103-P #108, P #111-P #118, P #120, P #121, P #123-P #218, P #220, P #224-P #231, P #254, P #258, P #259, P #268, P #270, P #285-P #288, P #302, P #495, P #496, P #498-P #539, P #541-P #551, P #554-P #586, P #590-P #594, P #628, P #682 and P #683.

According to other particular embodiments, the at least one allele is a “non-fat allele” or a “fat allele”, preferably the latter, for C10:0 of at least one polymorphism selected from the group consisting of P #3, P #8-P #10, P #16-P #20, P #49-P #51, P #56-P #62, P #64, P #69-P #72, P #74, P #76-P #78, P #80-P #96, P #104-P #109, P #111-P #115, P #117, P #118, P #120, P #121, P #127, P #128, P #132, P #133, P #136-P #142, P #148-P #153, P #156-P #166, P #224-P #226, P #285-P #288, P #297-P #302, P #490-P #494, P #501-P #514, P #517-P #539, P #541-P #547, P #551, P #554-P #580, P #585, P #586, P #589-P #593, P #628, P #889-P #891, P #899, P #900, P #902-P #916.

According to other particular embodiments, the at least one allele is a “non-fat allele” or a “fat allele”, preferably the latter, for C12:0 of at least one polymorphism selected from the group consisting of P #3, P #9, P #10, P #16-P #20, P #49-P #53, P #61, P #62, P #64, P #67, P #69-P #72, P #74, P #83, P #85-P #87, P #89-P #96, P #104-P #113, P #115, P #117-P #122, P #127, P #128, P #132, P #133, P #138, P #139, P #141, P #142, P #148-P #151, P #153, P #219, P #224-P #226, P #276, P #285-P #288, P #290-P #302, P #487-P #495, P #497, P #501-P #515, P #517-P #547, P #551, P #554-P #580, P #585, P #586, P #588-P #594, P #887-P #891, P #893-P #916.

According to other particular embodiments, the at least one allele is a “non-fat allele” or a “fat allele”, preferably the latter, for C14:0 of at least one polymorphism selected from the group consisting of P #9, P #10, P #17-P #19, P #34, P #69, P #70, P #83, P #85, P #117, P #120, P #121, P #127, P #128, P #132, P #133, P #139, P #141, P #148-P #151, P #153, P #224-P #226, P #275, P #276, P #279-P #281, P #284-P #302, P #483-P #494, P #497, P #501-P #514, P #517-P #539, P #541, P #543-P #580, P #584-P #594, P #889-P #892, P #899, P #900, P #902-P #916.

According to other particular embodiments, the at least one allele is a “non-fat allele” or a “fat allele”, preferably the latter, for C14:1 cis-9 of at least one polymorphism selected from the group consisting of P #243-P #246, P #314-P #332, P #334-P #340, P #346-P #352, P #354-P #363, P #366-P #392, P #394-P #396, P #398-P #410, P #412-P #475 and P #477-P #481.

It is to be understood that a “fat-allele” for a specific trait is indicative of increased amount of that trait in milk; and a “non-fat allele” for a specific trait is indicative of decreased amount of that trait in milk (“fat allele”, “non-fat allele” and the respective traits are specified in table 1).

According to particular embodiments, the (suitable) female (milk-producing) cattle comprises within its genome at least one nucleotide sequence selected from the group consisting of a) the nucleotide sequences set forth in any one of SEQ ID NOs: 1 to 916, such as SEQ ID NOs: 1 to 310; and b) nucleotide sequences which are derived from any one of SEQ ID NOs: 1 to 916, such as SEQ ID NOs: 1 to 310, by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition.

In one embodiment according to the present invention, the nucleotide substitution(s) and/or nucleotide deletions (preferably substitutions) are not in the polymorphic site (i.e. position 60) of the derived sequence.

According to particular embodiments, the (suitable) female (milk-producing) cattle comprises within its genome at least one nucleotide sequence selected from the group consisting of a) the nucleotide sequences set forth in any one of SEQIDNOs 33, 241-248, 303-312, 314-344, 346-475 and, 477-481 (C16:0); and b) nucleotide sequences which are derived from any one of SEQIDNOs 33, 241-248, 303-312, 314-344, 346-475 and, 477-481 (C16:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “non-fat allele” for C16:0.

According to particular embodiments, the (suitable) female (milk-producing) cattle comprises within its genome at least one nucleotide sequence selected from the group consisting of a) the nucleotide sequences set forth in any one of SEQ ID NOs: 19, 34, 38, 39, 141, 148, 153, 233-240, 242-246, 271-278, 280-283, 285, 289-302, 311-334, 339, 340, 343-475, 477-481, 484-494, 497, 543, 588, 589, 916, 911, 14, 37, 903 and 7 (C18:1); and b) nucleotide sequences which are derived from any one of SEQ ID NOs: 19, 34, 38, 39, 141, 148, 153, 233-240, 242-246, 271-278, 280-283, 285, 289-302, 311-334, 339, 340, 343-475, 477-481, 484-494, 497, 543, 588, 589, 916, 911, 14, 37, 903 and 7 (C18:1) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C18:1.

According to particular embodiments, the (suitable) female (milk-producing) cattle comprises within its genome at least one nucleotide sequence selected from the group consisting of a) the nucleotide sequences set forth in any one of SEQ ID NOs: 243-246, 314-332, 334-340, 346-352, 354-363, 366-392, 394-396, 398-410, 412-475 and 477-481 (C14:1 cis-9); and b) nucleotide sequences which are derived from any one of SEQ ID NOs: 243-246, 314-332, 334-340, 346-352, 354-363, 366-392, 394-396, 398-410, 412-475 and 477-481 (C14:1 cis-9) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C14:1 cis-9.

According to particular embodiments, the (suitable) female (milk-producing) cattle comprises within its genome at least one nucleotide sequence selected from the group consisting of a) the nucleotide sequences set forth in any one of SEQ ID NOs: 1, 12, 15-17, 24, 41, 63, 64, 69-71, 74-79, 83, 85-87, 89-93, 97-103, 112-118, 120, 121, 123-135, 138-141, 148, 153-168, 218, 220-226, 249, 250, 253-260, 267, 495, 499, 501, 503-514, 517-539, 541, 542, 544-547, 554-585, 590-592, 595-597, 599-614, 617-621, 623-652, 654-669, 672-674, 676-678, 681, 682, 688-744, 746-769, 771-813, 815, 818-824, 826-831 and 872 (C6:0); and b) nucleotide sequences which are derived from any one of SEQ ID NOs: 1, 12, 15-17, 24, 41, 63, 64, 69-71, 74-79, 83, 85-87, 89-93, 97-103, 112-118, 120, 121, 123-135, 138-141, 148, 153-168, 218, 220-226, 249, 250, 253-260, 267, 495, 499, 501, 503-514, 517-539, 541, 542, 544-547, 554-585, 590-592, 595-597, 599-614, 617-621, 623-652, 654-669, 672-674, 676-678, 681, 682, 688-744, 746-769, 771-813, 815, 818-824, 826-831 and 872 (C6:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C6:0.

According to particular embodiments, the (suitable) female (milk-producing) cattle comprises within its genome at least one nucleotide sequence selected from the group consisting of a) the nucleotide sequences set forth in any one of SEQ ID NOs: 1, 10-12, 16-19, 40-45, 48, 49, 51, 56-60, 63-66, 68-93, 97, 99-101, 103-108, 111-118, 120, 121, 123-218, 220, 224-231, 254, 258, 259, 268, 270, 285-288, 302, 495, 496, 498-539, 541-551, 554-586, 590-594, 628, 682 and 683 (C8:0); and b) nucleotide sequences which are derived from any one of SEQ ID NOs: 1, 10-12, 16-19, 40-45, 48, 49, 51, 56-60, 63-66, 68-93, 97, 99-101, 103-108, 111-118, 120, 121, 123-218, 220, 224-231, 254, 258, 259, 268, 270, 285-288, 302, 495, 496, 498-539, 541-551, 554-586, 590-594, 628, 682 and 683 (C8:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C8:0.

According to particular embodiments, the (suitable) female (milk-producing) cattle comprises within its genome at least one nucleotide sequence selected from the group consisting of a) the nucleotide sequences set forth in any one of SEQ ID NOs: 3, 8-10, 16-20, 49-51, 56-62, 64, 69-72, 74, 76-78, 80-96, 104-109, 111-115, 117, 118, 120, 121, 127, 128, 132, 133, 136-142, 148-153, 156-166, 224-226, 285-288, 297-302, 490-494, 501-514, 517-539, 541-547, 551, 554-580, 585, 586, 589-593, 628, 889-891, 899, 900, 902-916 (C10:0); and b) nucleotide sequences which are derived from any one of SEQ ID NOs: 3, 8-10, 16-20, 49-51, 56-62, 64, 69-72, 74, 76-78, 80-96, 104-109, 111-115, 117, 118, 120, 121, 127, 128, 132, 133, 136-142, 148-153, 156-166, 224-226, 285-288, 297-302, 490-494, 501-514, 517-539, 541-547, 551, 554-580, 585, 586, 589-593, 628, 889-891, 899, 900, 902-916 (C10:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C10:0.

According to particular embodiments, the (suitable) female (milk-producing) cattle comprises within its genome at least one nucleotide sequence selected from the group consisting of a) the nucleotide sequences set forth in any one of SEQ ID NOs: 3, 9, 10, 16-20, 49-53, 61, 62, 64, 67, 69-72, 74, 83, 85-87, 89-96, 104-113, 115, 117-122, 127, 128, 132, 133, 138, 139, 141, 142, 148-151, 153, 219, 224-226, 276, 285-288, 290-302, 487-495, 497, 501-515, 517-547, 551, 554-580, 585, 586, 588-594, 887-891, 893-916 (C12:0); and b) nucleotide sequences which are derived from any one of SEQ ID NOs: 3, 9, 10, 16-20, 49-53, 61, 62, 64, 67, 69-72, 74, 83, 85-87, 89-96, 104-113, 115, 117-122, 127, 128, 132, 133, 138, 139, 141, 142, 148-151, 153, 219, 224-226, 276, 285-288, 290-302, 487-495, 497, 501-515, 517-547, 551, 554-580, 585, 586, 588-594, 887-891, 893-916 (C12:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C12:0.

According to particular embodiments, the (suitable) female (milk-producing) cattle comprises within its genome at least one nucleotide sequence selected from the group consisting of a) the nucleotide sequences set forth in any one of SEQ ID NOs: 9, 10, 17-19, 34, 69, 70, 83, 85, 117, 120, 121, 127, 128, 132, 133, 139, 141, 148-151, 153, 224-226, 275, 276, 279-281, 284-302, 483-494, 497, 501-514, 517-539, 541, 543-580, 584-594, 889-892, 899, 900, 902-916 (C14:0); and b) nucleotide sequences which are derived from any one of SEQ ID NOs: 9, 10, 17-19, 34, 69, 70, 83, 85, 117, 120, 121, 127, 128, 132, 133, 139, 141, 148-151, 153, 224-226, 275, 276, 279-281, 284-302, 483-494, 497, 501-514, 517-539, 541, 543-580, 584-594, 889-892, 899, 900, 902-916 (C14:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C14:0.

In Vitro Method for Selective Breeding

The present invention provides in a fourth aspect a method for selective breeding of a cattle, the method comprises:

in vitro fertilizing the non-human ovum defined in the second aspect of the present invention using the non-human semen or non-human sperm defined in the second aspect of the present invention; and

implanting the in vitro fertilized non-human ovum in the uterus of a (suitable) female (milk-producing) cattle.

An alternative aspect of the present invention relates to a method for selective breeding of a cattle, the method comprises:

providing non-human ovum such as cattle ovum, comprising within its genome at least one allele which in a female milk-producing cattle is indicative of a desired milk fatty acid composition;

providing non-human semen or non-human sperm comprising within its genome at least one allele which in a female milk-producing cattle is indicative of a desired milk fatty acid composition;

in vitro fertilizing the non-human ovum using the non-human semen or non-human sperm; and

implanting the in vitro fertilized non-human ovum in the uterus of a (suitable) female (milk-producing) cattle.

In vitro fertilization is a process by which an ovum is fertilized by semen or sperm outside the body. The process typically involves monitoring and stimulating a cattle's ovulatory process, removing an ovum from the animal's ovaries and letting semen or sperm fertilize them in a liquid in a laboratory. The fertilized ovum is typically cultured for some days, e.g. 2-6 days, in a growth medium and is then implanted in the same or another female cattle's uterus, with the intention of establishing a successful pregnancy.

In the context of in vitro fertilization, a suitable female (milk-producing) cattle is a cattle that is capable of being implanted with an in vitro fertilized non-human ovum in the sense that the in vitro fertilized non-human ovum develops into a new individual organisms within the body of the suitable female (milk-producing) cattle.

According to particular embodiments, the method comprises:

providing a non-human semen or non-human sperm as defined in the second aspect of the present invention;

providing a non-human ovum as defined in the second aspect of the present invention;

in vitro fertilizing the ovum using the non-human semen or non-human sperm; and

implanting the in vitro fertilized ovum in the uterus of a (suitable) female (milk-producing) cattle.

Cattle

The present invention provides in a fifth aspect, a cattle obtainable by the method according to the first aspect of the present invention, the method according to the third aspect of the present invention or the method according to the fourth aspect of the present invention.

The present invention provides in a sixth aspect, a cattle comprising within its genome at least one allele, such as two, three or four alleles, which in a female milk-producing cattle is indicative of a desired milk fatty acid composition.

The at least one allele referred to in the sixth aspect of the present invention refers to at least one allele which determines a specific phenotype only in female milk-producing cattle. Said cattle may be male or female, but the at least one allele referred to will only determine the specific phenotype in female milk-producing cattle.

A female milk-producing cattle according to the fifth or sixth aspect of the present invention will have a desired milk fatty acid composition. A male cattle according to the fifth or sixth aspect of the present invention possesses a genotype which in a female milk-producing cattle is indicative of a desired milk fatty acid composition and may therefore find use as a breeding animal or as a producer of semen and/or sperm which may be used in various breeding programs.

Since the at least one allele referred to above determines a specific phenotype only in female milk-producing cattle, it has been decided to differentiate between a female cattle, which not necessarily produces milk, and a female milk-producing cattle, which produces milk by definition.

The cattle referred to in the above method may be male or female. In one embodiment according to the present invention, said cattle is male. In another embodiment according to the present invention, said cattle is female, preferably a female milk-producing cattle.

In one embodiment according to the present invention, said cattle is selected from the group consisting of Norwegian Red cattle.

In one embodiment according to the present invention, said female milk-producing cattle is selected from the group consisting of female milk-producing Norwegian Red cattle.

In one embodiment according to the present invention, the cattle is heterozygous with respect to the at least one allele which in a female milk-producing cattle is indicative of a desired milk fatty acid composition. In another embodiment according to the present invention, the cattle is homozygous with respect to the at least one allele which in a female milk-producing cattle is indicative of a desired milk fatty acid composition.

In one embodiment according to the sixth aspect of the present invention, the at least one allele is an allele of at least one polymorphism, such as at least one single nucleotide polymorphism (SNP). In a preferred embodiment, the at least one polymorphism is selected from the polymorphisms listed in table 1.

In another embodiment according to the sixth aspect of the present invention, the at least one allele is an allele of at least one polymorphism selected from the group consisting of P #1 to P #916, such as P #1 to P #310. In another embodiment according to the sixth aspect of the present invention, the at least one allele is an allele of at least one polymorphism selected from the group consisting of P #33, P #241-P #248, P #303-P #312, P #314-P #344, P #346-P #475 and, P #477-P #481 (C16:0), preferably the at least one allele represents a “non-fat allele” for C16:0. In another embodiment according to the sixth aspect of the present invention, the at least one allele is an allele of at least one polymorphism selected from the group consisting of P #19, P #34, P #38, P #39, P #141, P #148, P #153, P #233-P #240, P #242-P #246, P #271-P #278, P #280-P #283, P #285, P #289-P #302, P #311-P #334, P #339, P #340, P #343-P #475, P #477-P #481, P #484-P #494, P #497, P #543, P #588, P #589, P #916, P #911, P #14, P #37, P #903 and P #7 (C18:1), preferably the at least one allele represents a “fat allele” for C18:1. In another embodiment according to the sixth aspect of the present invention, the at least one allele is an allele of at least one polymorphism selected from the group consisting of P #1, P #12, P #15-P #17, P #24, P #41, P #63, P #64, P #69-P #71, P #74-P #79, P #83, P #85-P #87, P #89-P #93, P #97-P #103, P #112-P #118, P #120, P #121, P #123-P #135, P #138-P #141, P #148, P #153-P #168, P #218, P #220-P #226, P #249, P #250, P #253-P #260, P #267, P #495, P #499, P #501, P #503-P #514, P #517-P #539, P #541, P #542, P #544-P #547, P #554-P #585, P #590-P #592, P #595-P #597, P #599-P #614, P #617-P #621, P #623-P #652, P #654-P #669, P #672-P #674, P #676-P #678, P #681, P #682, P #688-P #744, P #746-P #769, P #771-P #813, P #815, P #818-P #824, P #826-P #831 and P #872 (C6:0), preferably the at least one allele represents a “fat allele” for C6:0. In another embodiment according to the sixth aspect of the present invention, the at least one allele is an allele of at least one polymorphism selected from the group consisting of P #1, P #10-P #12, P #16-P #19, P #40-P #45, P #48, P #49, P #51, P #56-P #60, P #63-P #66, P #68-P #93, P #97, P #99-P #101, P #103-P #108, P #111-P #118, P #120, P #121, P #123-P #218, P #220, P #224-P #231, P #254, P #258, P #259, P #268, P #270, P #285-P #288, P #302, P #495, P #496, P #498-P #539, P #541-P #551, P #554-P #586, P #590-P #594, P #628, P #682 and P #683 (C8:0), preferably the at least one allele represents a “fat allele” for C8:0. In another embodiment according to the sixth aspect of the present invention, the at least one allele is an allele of at least one polymorphism selected from the group consisting of P #3, P #8-P #10, P #16-P #20, P #49-P #51, P #56-P #62, P #64, P #69-P #72, P #74, P #76-P #78, P #80-P #96, P #104-P #109, P #111-P #115, P #117, P #118, P #120, P #121, P #127, P #128, P #132, P #133, P #136-P #142, P #148-P #153, P #156-P #166, P #224-P #226, P #285-P #288, P #297-P #302, P #490-P #494, P #501-P #514, P #517-P #539, P #541-P #547, P #551, P #554-P #580, P #585, P #586, P #589-P #593, P #628, P #889-P #891, P #899, P #900, P #902-P #916 (C10:0), preferably the at least one allele represents a “fat allele” for C10:0. In another embodiment according to the sixth aspect of the present invention, the at least one allele is an allele of at least one polymorphism selected from the group consisting of P #3, P #9, P #10, P #16-P #20, P #49-P #53, P #61, P #62, P #64, P #67, P #69-P #72, P #74, P #83, P #85-P #87, P #89-P #96, P #104-P #113, P #115, P #117-P #122, P #127, P #128, P #132, P #133, P #138, P #139, P #141, P #142, P #148-P #151, P #153, P #219, P #224-P #226, P #276, P #285-P #288, P #290-P #302, P #487-P #495, P #497, P #501-P #515, P #517-P #547, P #551, P #554-P #580, P #585, P #586, P #588-P #594, P #887-P #891, P #893-P #916 (C12:0), preferably the at least one allele represents a “fat allele” for C12:0. In another embodiment according to the sixth aspect of the present invention, the at least one allele is an allele of at least one polymorphism selected from the group consisting of P #9, P #10, P #17-P #19, P #34, P #69, P #70, P #83, P #85, P #117, P #120, P #121, P #127, P #128, P #132, P #133, P #139, P #141, P #148-P #151, P #153, P #224-P #226, P #275, P #276, P #279-P #281, P #284-P #302, P #483-P #494, P #497, P #501-P #514, P #517-P #539, P #541, P #543-P #580, P #584-P #594, P #889-P #892, P #899, P #900, P #902-P #916 (C14:0), preferably the at least one allele represents a “fat allele” for C14:0. In another embodiment according to the sixth aspect of the present invention, the at least one allele is an allele of at least one polymorphism selected from the group consisting of P #243-P #246, P #314-P #332, P #334-P #340, P #346-P #352, P #354-P #363, P #366-P #392, P #394-P #396, P #398-P #410, P #412-P #475 and P #477-P #481 (C14:1 cis-9), preferably the at least one allele represents a “fat allele” for C14:1 cis-9.

In another embodiment according to the sixth aspect of the present invention, a desired milk fatty acid composition is decreased amount of C16:0 in milk; and/or increased amount of C18:1 in milk; and/or increased amount of C14:1 cis-9 in milk; and/or increased amount of one or more fatty acids selected from the group consisting of C6:0, C8:0, C10:0, C12:0 and C14:0 in milk; and/or optionally any combination thereof.

According to other particular embodiments, the at least one allele is a “fat allele” or a “non-fat allele”, preferably the latter, for C16:0 of at least one polymorphism selected from the group consisting of P #33, P #241-P #248, P #303-P #312, P #314-P #344, P #346-P #475 and, P #477-P #481.

According to other particular embodiments, the at least one allele is a “non-fat allele” or a “fat allele”, preferably the latter, for C18:1 of at least one polymorphism selected from the group consisting of P #19, P #34, P #38, P #39, P #141, P #148, P #153, P #233-P #240, P #242-P #246, P #271-P #278, P #280-P #283, P #285, P #289-P #302, P #311-P #334, P #339, P #340, P #343-P #475, P #477-P #481, P #484-P #494, P #497, P #543, P #588, P #589, P #916, P #911, P #14, P #37, P #903 and P #7.

According to other particular embodiments, the at least one allele is a “non-fat allele” or a “fat allele”, preferably the latter, for C6:0 of at least one polymorphism selected from the group consisting of P #1, P #12, P #15-P #17, P #24, P #41, P #63, P #64, P #69-P #71, P #74-P #79, P #83, P #85-P #87, P #89-P #93, P #97-P #103, P #112-P #118, P #120, P #121, P #123-P #135, P #138-P #141, P #148, P #153-P #168, P #218, P #220-P #226, P #249, P #250, P #253-P #260, P #267, P #495, P #499, P #501, P #503-P #514, P #517-P #539, P #541, P #542, P #544-P #547, P #554-P #585, P #590-P #592, P #595-P #597, P #599-P #614, P #617-P #621, P #623-P #652, P #654-P #669, P #672-P #674, P #676-P #678, P #681, P #682, P #688-P #744, P #746-P #769, P #771-P #813, P #815, P #818-P #824, P #826-P #831 and P #872.

According to other particular embodiments, the at least one allele is a “non-fat allele” or a “fat allele”, preferably the latter, for C8:0 of at least one polymorphism selected from the group consisting of P #1, P #10-P #12, P #16-P #19, P #40-P #45, P #48, P #49, P #51, P #56-P #60, P #63-P #66, P #68-P #93, P #97, P #99-P #101, P #103-P #108, P #111-P #118, P #120, P #121, P #123-P #218, P #220, P #224-P #231, P #254, P #258, P #259, P #268, P #270, P #285-P #288, P #302, P #495, P #496, P #498-P #539, P #541-P #551, P #554-P #586, P #590-P #594, P #628, P #682 and P #683.

According to other particular embodiments, the at least one allele is a “non-fat allele” or a “fat allele”, preferably the latter, for C10:0 of at least one polymorphism selected from the group consisting of P #3, P #8-P #10, P #16-P #20, P #49-P #51, P #56-P #62, P #64, P #69-P #72, P #74, P #76-P #78, P #80-P #96, P #104-P #109, P #111-P #115, P #117, P #118, P #120, P #121, P #127, P #128, P #132, P #133, P #136-P #142, P #148-P #153, P #156-P #166, P #224-P #226, P #285-P #288, P #297-P #302, P #490-P #494, P #501-P #514, P #517-P #539, P #541-P #547, P #551, P #554-P #580, P #585, P #586, P #589-P #593, P #628, P #889-P #891, P #899, P #900, P #902-P #916.

According to other particular embodiments, the at least one allele is a “non-fat allele” or a “fat allele”, preferably the latter, for C12:0 of at least one polymorphism selected from the group consisting of P #3, P #9, P #10, P #16-P #20, P #49-P #53, P #61, P #62, P #64, P #67, P #69-P #72, P #74, P #83, P #85-P #87, P #89-P #96, P #104-P #113, P #115, P #117-P #122, P #127, P #128, P #132, P #133, P #138, P #139, P #141, P #142, P #148-P #151, P #153, P #219, P #224-P #226, P #276, P #285-P #288, P #290-P #302, P #487-P #495, P #497, P #501-P #515, P #517-P #547, P #551, P #554-P #580, P #585, P #586, P #588-P #594, P #887-P #891, P #893-P #916.

According to other particular embodiments, the at least one allele is a “non-fat allele” or a “fat allele”, preferably the latter, for C14:0 of at least one polymorphism selected from the group consisting of P #9, P #10, P #17-P #19, P #34, P #69, P #70, P #83, P #85, P #117, P #120, P #121, P #127, P #128, P #132, P #133, P #139, P #141, P #148-P #151, P #153, P #224-P #226, P #275, P #276, P #279-P #281, P #284-P #302, P #483-P #494, P #497, P #501-P #514, P #517-P #539, P #541, P #543-P #580, P #584-P #594, P #889-P #892, P #899, P #900, P #902-P #916.

According to other particular embodiments, the at least one allele is a “non-fat allele” or a “fat allele”, preferably the latter, for C14:1 cis-9 of at least one polymorphism selected from the group consisting of P #243-P #246, P #314-P #332, P #334-P #340, P #346-P #352, P #354-P #363, P #366-P #392, P #394-P #396, P #398-P #410, P #412-P #475 and P #477-P #481.

It is to be understood that a “fat-allele” for a specific trait is indicative of increased amount of that trait in milk; and a “non-fat allele” for a specific trait is indicative of decreased amount of that trait in milk (“fat allele”, “non-fat allele” and the respective traits are specified in table 1).

According to particular embodiments, the cattle comprises within its genome at least one nucleotide sequence selected from the group consisting of a) the nucleotide sequences set forth in any one of SEQ ID NOs: 1 to 916, such as SEQ ID NOs: 1 to 310; and b) nucleotide sequences which are derived from any one of SEQ ID NOs: 1 to 916, such as SEQ ID NOs: 1 to 310, by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition.

In one embodiment according to the present invention, the nucleotide substitution(s) and/or nucleotide deletions (preferably substitutions) are not in the polymorphic site (i.e. position 60) of the derived sequence.

According to particular embodiments, the cattle comprises within its genome at least one nucleotide sequence selected from the group consisting of a) the nucleotide sequences set forth in any one of SEQIDNOs 33, 241-248, 303-312, 314-344, 346-475 and, 477-481 (C16:0); and b) nucleotide sequences which are derived from any one of SEQIDNOs 33, 241-248, 303-312, 314-344, 346-475 and, 477-481 (C16:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “non-fat allele” for C16:0.

According to particular embodiments, the cattle comprises within its genome at least one nucleotide sequence selected from the group consisting of a) the nucleotide sequences set forth in any one of SEQ ID NOs: 19, 34, 38, 39, 141, 148, 153, 233-240, 242-246, 271-278, 280-283, 285, 289-302, 311-334, 339, 340, 343-475, 477-481, 484-494, 497, 543, 588, 589, 916, 911, 14, 37, 903 and 7 (C18:1); and b) nucleotide sequences which are derived from any one of SEQ ID NOs: 19, 34, 38, 39, 141, 148, 153, 233-240, 242-246, 271-278, 280-283, 285, 289-302, 311-334, 339, 340, 343-475, 477-481, 484-494, 497, 543, 588, 589, 916, 911, 14, 37, 903 and 7 (C18:1) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C18:1.

According to particular embodiments, the cattle comprises within its genome at least one nucleotide sequence selected from the group consisting of a) the nucleotide sequences set forth in any one of SEQ ID NOs: 243-246, 314-332, 334-340, 346-352, 354-363, 366-392, 394-396, 398-410, 412-475 and 477-481 (C14:1 cis-9); and b) nucleotide sequences which are derived from any one of SEQ ID NOs: 243-246, 314-332, 334-340, 346-352, 354-363, 366-392, 394-396, 398-410, 412-475 and 477-481 (C14:1 cis-9) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C14:1 cis-9.

According to particular embodiments, the cattle comprises within its genome at least one nucleotide sequence selected from the group consisting of a) the nucleotide sequences set forth in any one of SEQ ID NOs: 1, 12, 15-17, 24, 41, 63, 64, 69-71, 74-79, 83, 85-87, 89-93, 97-103, 112-118, 120, 121, 123-135, 138-141, 148, 153-168, 218, 220-226, 249, 250, 253-260, 267, 495, 499, 501, 503-514, 517-539, 541, 542, 544-547, 554-585, 590-592, 595-597, 599-614, 617-621, 623-652, 654-669, 672-674, 676-678, 681, 682, 688-744, 746-769, 771-813, 815, 818-824, 826-831 and 872 (C6:0); and b) nucleotide sequences which are derived from any one of SEQ ID NOs: 1, 12, 15-17, 24, 41, 63, 64, 69-71, 74-79, 83, 85-87, 89-93, 97-103, 112-118, 120, 121, 123-135, 138-141, 148, 153-168, 218, 220-226, 249, 250, 253-260, 267, 495, 499, 501, 503-514, 517-539, 541, 542, 544-547, 554-585, 590-592, 595-597, 599-614, 617-621, 623-652, 654-669, 672-674, 676-678, 681, 682, 688-744, 746-769, 771-813, 815, 818-824, 826-831 and 872 (C6:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C6:0.

According to particular embodiments, the cattle comprises within its genome at least one nucleotide sequence selected from the group consisting of a) the nucleotide sequences set forth in any one of SEQ ID NOs: 1, 10-12, 16-19, 40-45, 48, 49, 51, 56-60, 63-66, 68-93, 97, 99-101, 103-108, 111-118, 120, 121, 123-218, 220, 224-231, 254, 258, 259, 268, 270, 285-288, 302, 495, 496, 498-539, 541-551, 554-586, 590-594, 628, 682 and 683 (C8:0); and b) nucleotide sequences which are derived from any one of SEQ ID NOs: 1, 10-12, 16-19, 40-45, 48, 49, 51, 56-60, 63-66, 68-93, 97, 99-101, 103-108, 111-118, 120, 121, 123-218, 220, 224-231, 254, 258, 259, 268, 270, 285-288, 302, 495, 496, 498-539, 541-551, 554-586, 590-594, 628, 682 and 683 (C8:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C8:0.

According to particular embodiments, the cattle comprises within its genome at least one nucleotide sequence selected from the group consisting of a) the nucleotide sequences set forth in any one of SEQ ID NOs: 3, 8-10, 16-20, 49-51, 56-62, 64, 69-72, 74, 76-78, 80-96, 104-109, 111-115, 117, 118, 120, 121, 127, 128, 132, 133, 136-142, 148-153, 156-166, 224-226, 285-288, 297-302, 490-494, 501-514, 517-539, 541-547, 551, 554-580, 585, 586, 589-593, 628, 889-891, 899, 900, 902-916 (C10:0); and b) nucleotide sequences which are derived from any one of SEQ ID NOs: 3, 8-10, 16-20, 49-51, 56-62, 64, 69-72, 74, 76-78, 80-96, 104-109, 111-115, 117, 118, 120, 121, 127, 128, 132, 133, 136-142, 148-153, 156-166, 224-226, 285-288, 297-302, 490-494, 501-514, 517-539, 541-547, 551, 554-580, 585, 586, 589-593, 628, 889-891, 899, 900, 902-916 (C10:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C10:0.

According to particular embodiments, the cattle comprises within its genome at least one nucleotide sequence selected from the group consisting of a) the nucleotide sequences set forth in any one of SEQ ID NOs: 3, 9, 10, 16-20, 49-53, 61, 62, 64, 67, 69-72, 74, 83, 85-87, 89-96, 104-113, 115, 117-122, 127, 128, 132, 133, 138, 139, 141, 142, 148-151, 153, 219, 224-226, 276, 285-288, 290-302, 487-495, 497, 501-515, 517-547, 551, 554-580, 585, 586, 588-594, 887-891, 893-916 (C12:0); and b) nucleotide sequences which are derived from any one of SEQ ID NOs: 3, 9, 10, 16-20, 49-53, 61, 62, 64, 67, 69-72, 74, 83, 85-87, 89-96, 104-113, 115, 117-122, 127, 128, 132, 133, 138, 139, 141, 142, 148-151, 153, 219, 224-226, 276, 285-288, 290-302, 487-495, 497, 501-515, 517-547, 551, 554-580, 585, 586, 588-594, 887-891, 893-916 (C12:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C12:0.

According to particular embodiments, the cattle comprises within its genome at least one nucleotide sequence selected from the group consisting of a) the nucleotide sequences set forth in any one of SEQ ID NOs: 9, 10, 17-19, 34, 69, 70, 83, 85, 117, 120, 121, 127, 128, 132, 133, 139, 141, 148-151, 153, 224-226, 275, 276, 279-281, 284-302, 483-494, 497, 501-514, 517-539, 541, 543-580, 584-594, 889-892, 899, 900, 902-916 (C14:0); and b) nucleotide sequences which are derived from any one of SEQ ID NOs: 9, 10, 17-19, 34, 69, 70, 83, 85, 117, 120, 121, 127, 128, 132, 133, 139, 141, 148-151, 153, 224-226, 275, 276, 279-281, 284-302, 483-494, 497, 501-514, 517-539, 541, 543-580, 584-594, 889-892, 899, 900, 902-916 (C14:0) by 1 to 30, such as 1 to 20, 1 to 15, 1 to 10, 1 to 5, 1 to 3 or 1 to 2 nucleotide substitutions and/or deletions (preferably substitutions);

wherein

the one or more nucleotides at position 60 of said nucleotide sequences is one or more nucleotides corresponding to the allele which in a female milk-producing cattle is indicative of a desired milk fatty acid composition. In one embodiment, the one or more nucleotides at position 60 of the nucleotide sequences correspond to the “fat allele” for C14:0.

Milk

Bovine milk is widely regarded as a valuable food source in human nutrition, and serves as an important source of proteins, minerals, vitamins and fats in western diets. In addition to being an important source of energy, the milk fat contains valuable fat-soluble vitamins and bio-active lipid components. Of the roughly 400 different fatty acids found in Bovine milk, only around 15 are present at the 1% level or higher.

The present invention provides female milk-producing cattle which herein have been shown to be associated with a desired milk fatty acid composition.

Thus, a seventh aspect of the present invention, relates to milk produced by the female milk-producing cattle according to the fifth or sixth aspect of the present invention.

Use

The present invention provides in an eighth aspect, use of an (isolated) nucleic acid molecule in an in vitro method for determining the presence of at least one allele, which in a female milk-producing cattle is indicative of a desired milk fatty acid composition, within the genome of a cattle;

wherein

the (isolated) nucleic acid molecule comprises at least one nucleotide sequence selected from the group consisting of a) a nucleotide sequence set forth in any one of SEQ ID NO: 1 to 916, such as SEQ ID NOs: 1 to 310; b) a nucleotide sequence derived from any one of SEQ ID NO: 1 to 916, such as SEQ ID NOs: 1 to 310, by 1 to 30, such as 1 to 20, 1 to 10, 1 to 5, 1 to 3 or 1 to 2 nucleotide substitutions and/or deletions (preferably substitutions); and c) complements to a) and b); the one or more nucleotides at position 60 of said nucleotide sequences being selected from the two alternative forms of the allele to be determined.

It is to be understood that the at least one allele is an allele of at least one polymorphism selected from the polymorphisms listed in table 1.

If the at least one allele to be determined is an allele of P #1, then the isolated nucleic acid molecule comprises a nucleotide sequence selected from the group consisting of a) a nucleotide sequence set forth in SEQ ID NO: 1; b) a nucleotide sequence derived from SEQ ID NO: 1 by 1 to 30, such as 1 to 20, 1 to 10, 1 to 5, 1 to 3 or 1 to 2 nucleotide substitutions and/or deletions (preferably substitutions); and c) complements to a) and b). If the at least one allele to be determined corresponds to the “fat allele” of P #1, the nucleotide at position 60 of SEQIDNO1 is a guanine. If the at least one allele to be determined corresponds to the “non-fat allele” of P #1, the nucleotide at position 60 of SEQIDNO1 is an adenine.

If the at least one allele to be determined is an allele of P #10, then the isolated nucleic acid molecule comprises a nucleotide sequence selected from the group consisting of a) a nucleotide sequence set forth in SEQ ID NO: 10; b) a nucleotide sequence derived from SEQ ID NO: 10 by 1 to 30, such as 1 to 20, 1 to 10, 1 to 5, 1 to 3 or 1 to 2 nucleotide substitutions and/or deletions (preferably substitutions); and c) complements to a) and b).

In view of the above examples and the information provided in table 1 and table 2, a man skilled in the art will easily know what nucleic acid molecule to use in order to determine the presence of an allele of a polymorphism selected from the polymorphisms listed in table 1.

The nucleic acid molecule may have a length of at least 119 nucleotides, such as at least 120 nucleotides, at least 121 nucleotides, at least 130 nucleotides or at least 140 nucleotides or at least 150 nucleotides, at least 160 nucleotides or even more than 160 nucleotides.

According to certain embodiments, the nucleic acid molecule has a length from 119 nucleotides to 400 nucleotides, such as from 119 nucleotides to 300 nucleotides or from 119 to 200 nucleotides, e.g. from 119 to 150 nucleotides.

According to certain embodiments, the nucleic acid molecule has a length from 120 nucleotides to 400 nucleotides, such as from 120 nucleotides to 300 nucleotides or from 120 to 200 nucleotides, e.g. from 120 to 150 nucleotides.

According to certain embodiments, the nucleic acid molecule has a length from 121 nucleotides to 400 nucleotides, such as from 121 nucleotides to 300 nucleotides or from 121 to 200 nucleotides, e.g. from 121 to 150 nucleotides.

It is to be understood that the above use involves analyzing a biological sample from a cattle for the presence of at least one allele, which in a female milk-producing cattle is indicative of a desired milk fatty acid composition.

The present invention provides in a ninth aspect, use of an (isolated) oligonucleotide in an in vitro method for determining the presence of at least one allele, which in a female milk-producing cattle is indicative of a desired milk fatty acid composition, within the genome of a cattle;

wherein the (isolated) oligonucleotide comprises at least 8 contiguous nucleotides of a nucleotide sequence selected from the group consisting of a) a nucleotide sequence set forth in any one of SEQ ID NO: 1 to 916, such as SEQ ID NOs: 1 to 310; b) a nucleotide sequence derived from any one of SEQ ID NO: 1 to 916, such as SEQ ID NOs: 1 to 310, by 1 to 30, such as 1 to 20, 1 to 10, 1 to 5, 1 to 3 or 1 to 2 nucleotide substitutions and/or deletions (preferably substitutions); and c) complements to a) and b); said at least 8 contiguous nucleotides include the one or more nucleotides at position 60 of a) or b); and the one or more nucleotides at position 60 of said nucleotide sequences being selected from the two alternative forms of the allele to be determined.

It is to be understood that the at least one allele is an allele of at least one polymorphism selected from the polymorphisms listed in table 1.

If the at least one allele to be determined is an allele of P #1, then the isolated oligonucleotide comprises at least 8 contiguous nucleotides of a nucleotide sequence selected from the group consisting of a) a nucleotide sequence set forth in SEQ ID NO: 1; b) a nucleotide sequence derived from SEQ ID NO: 1 by 1 to 30, such as 1 to 20, 1 to 10, 1 to 5, 1 to 3 or 1 to 2 nucleotide substitutions and/or deletions (preferably substitutions); and c) complements to a) and b); said at least 8 contiguous nucleotides include the one or more nucleotides at position 60 of a) or b).

If the at least one allele to be determined is an allele of P #10, then the isolated oligonucleotide comprises at least 8 contiguous nucleotides of a nucleotide sequence selected from the group consisting of a) a nucleotide sequence set forth in SEQ ID NO: 10; b) a nucleotide sequence derived from SEQ ID NO: 10 by 1 to 30, such as 1 to 20, 1 to 10, 1 to 5, 1 to 3 or 1 to 2 nucleotide substitutions and/or deletions (preferably substitutions); and c) complements to a) and b); said at least 8 contiguous nucleotides include the one or more nucleotides at position 60 of a) or b).

In view of the above examples and the information provided in table 1 and table 2, a man skilled in the art will easily know what oligonucleotide to use in order to determine the presence of an allele of a polymorphism selected from the polymorphisms listed in table 1.

It is to be understood that the above use involves analyzing a biological sample from a cattle for the presence of at least one allele, which in a female milk-producing cattle is indicative of a desired milk fatty acid composition.

As used herein, an “oligonucleotide” is a plurality of joined nucleotides joined by native phosphodiester bonds, typically from 8 to 300 nucleotides in length.

According to certain embodiments, the oligonucleotide or complement thereof has a length of at least 8 nucleotides, such as at least 10 nucleotides.

According to certain embodiments, the oligonucleotide or complement thereof has a length of at least 15 nucleotides, such as at least 20 nucleotides.

According to certain embodiments, the oligonucleotide or complement thereof has a length of at least 30 nucleotides, such as at least 40 nucleotides.

According to certain embodiments, the oligonucleotide or complement thereof has a length of at least 50 nucleotides, such as at least 60 nucleotides.

According to certain embodiments, the oligonucleotide or complement thereof has a length of at least 70 nucleotides, such as at least 80 nucleotides.

According to certain embodiments, the oligonucleotide or complement thereof has a length of 30 to 200 nucleotides, such as 30 to 150 nucleotides.

According to certain embodiments, the oligonucleotide or complement thereof has a length of 30 to 100 nucleotides, such as 30 to 70 nucleotides.

According to certain embodiments, the oligonucleotide or complement thereof has a length of 30 to 100 nucleotides, such as 30 to 70 nucleotides.

According to certain embodiments, the oligonucleotide or complement thereof has a length of 30 to 50 nucleotides, such as 30 to 40 nucleotides.

According to certain embodiments, the oligonucleotide or complement thereof has a length of 8 to 200 nucleotides, such as 8 to 150 nucleotides.

According to certain embodiments, the oligonucleotide or complement thereof has a length of 8 to 100 nucleotides, such as 8 to 70 nucleotides.

According to certain embodiments, the oligonucleotide or complement thereof has a length of 8 to 100 nucleotides, such as 8 to 70 nucleotides.

According to certain embodiments, the oligonucleotide or complement thereof has a length of 8 to 50 nucleotides, such as 8 to 40 nucleotides.

According to certain embodiments, the oligonucleotide or complement thereof is a primer, such as a PCR primer.

According to certain embodiments, the oligonucleotide or complement thereof is a probe, such as a hybridization probe.

As used herein, “probes” and “primer” are isolated oligonucleotides of at least 8 nucleotides, such as at least 10 nucleotides, capable of hybridizing, preferably hybridizing under stringent conditions, to a target nucleic acid.

The term “hybridization stringency” refers to the degree to which mismatches are tolerated in a hybridization assay. The more stringent the conditions, the more likely mismatched heteroduplexes are to be forced apart, whereas less stringent hybridization conditions enhance the stability of mismatched heteroduplexes. In other words, increasing the stringency increases the specificity of the hybridization reaction. A person skilled in the art is able to select the hybridization conditions such that a desired level of stringency is achieved. Generally, the stringency may be increased by increasing temperatures (closer to the melting temperature (Tm) of the heteroduplex), lowering the salt concentrations, and using organic solvents. As known in the art, stringent hybridization conditions are sequence dependent and, thus, they are different under different experimental parameters.

The hybridization conditions can be chosen such that a single mismatch renders a heteroduplex unstable. Such hybridization conditions may be called as “highly stringent hybridization conditions”.

The Tm is the temperature (under defined ionic strength, pH, and DNA concentration) at which 50% of the target motifs are hybiridized with their matched binding units. Stringent conditions may be obtained by performing the hybridization in a temperature equal or close to the Tm for the probe in question.

Exemplary stringent hybridization conditions for short binding units include 6×SSC, 0.5% Tween20, and 20% form amide incubated at 37° C. in 600 rpm for one hour, followed by washing twice in TBS buffer containing 0.05% Tween20 at room temperature.

According to certain embodiments, the present invention provides a complement to the oligonucleotide specified above. Such complement may be used as a probe, such as a hybridization probe.

A probe or primer according to the present invention may have attached to it a detectable label or reporter molecule. Typical labels include radioactive isotopes, enzyme substrates, co-factors, ligands, chemiluminescent or fluorescent agents, haptens, and enyzmes. Methods for labelling and guidance in the choice of labels appropriate for various purposes are discussed, for example, in Sambrook et al. (In Molecular Cloning, A Laboratory Manual, CSHL, New York, 1989) and Ausubel et al. (In Current Protocols in Molecular Biology, John Wiley & Sons, New York, 1998). As a particular example, a probe or primer may include one fluorophor, such as an acceptor fluorophore or donor fluorophor. Such fluorophore may be attached at the 5′- or 3′ end of the probe/primer.

Probes are generally at least 15 nucleotides in length, such as at least 16, at least 17, at least 18, at least 19, at least 20, at least 25, at least 30, at least 35, at least 40, at least 45, at least 50, at least 55, at least 60, at least 65, at least 70, or more contiguous nucleotides complementary to the target nucleic acid molecule, such as 20 to 70 nucleotides, 20 to 60 nucleotides, 20 to 50 nucleotides, 20 to 40 nucleotides, or 20 to 30 nucleotides.

Primers are shorter in length. An oligonucleotide used as primer may be at least 10 nucleotides in length. The specificity of a primer increases with its length. Thus, for example, a primer that includes 30 consecutive nucleotides will anneal to a target sequence with a higher specificity that a corresponding primer of only 15 nucleotides. Thus, to obtain greater specificity, primers of the invention are at least 15 nucleotides in length, such as at least 16, at least 17, at least 18, at least 19, at least 20, at least 25, at least 30, at least 35, at least 40, at least 45, at least 50, at least 55, at least 60, at least 65, at least 70, or more contiguous nucleotides complementary to the target nucleic acid molecule, such as 15 to 70 nucleotides, 15 to 60 nucleotides, 15 to 50 nucleotides, 15 to 40 nucleotides, or 15 to 30 nucleotides. Primer pairs can be used for amplification of nucleic acid sequences, for example, by PCT, real-time-PCR, or other nucleic-acid amplification methods known in the art.

Method for Predicting

The present invention provides in an alternative aspect a method for predicting milk fatty acid composition in a female milk-producing cattle, the method comprises:

determining the presence of at least one allele, such as at least two, three or four alleles, which in a female milk-producing cattle is indicative of altered milk fatty acid composition, within the genome of said female milk-producing cattle.

In one embodiment according to the present invention, said female milk-producing cattle is female milk-producing cattle and in particular Norwegian Red cattle.

In one embodiment according to the alternative aspect of the present invention, the at least one allele is an allele of at least one polymorphism, such as at least one single nucleotide polymorphism (SNP). In a preferred embodiment, the at least one polymorphism is selected from the polymorphisms listed in table 1.

In another embodiment according to the alternative aspect of the present invention, the at least one allele is an allele of at least one polymorphism selected from the group consisting of P #1 to P #916, such as P #1 to P #310. In another embodiment according to the alternative aspect of the present invention, the at least one allele is an allele of at least one polymorphism selected from the group consisting of P #33, P #241-P #248, P #303-P #312, P #314-P #344, P #346-P #475 and, P #477-P #481 (C16:0), preferably the at least one allele represents a “non-fat allele” for C16:0. In another embodiment according to the alternative aspect of the present invention, the at least one allele is an allele of at least one polymorphism selected from the group consisting of P #19, P #34, P #38, P #39, P #141, P #148, P #153, P #233-P #240, P #242-P #246, P #271-P #278, P #280-P #283, P #285, P #289-P #302, P #311-P #334, P #339, P #340, P #343-P #475, P #477-P #481, P #484-P #494, P #497, P #543, P #588, P #589, P #916, P #911, P #14, P #37, P #903 and P #7 (C18:1), preferably the at least one allele represents a “fat allele” for C18:1. In another embodiment according to the alternative aspect of the present invention, the at least one allele is an allele of at least one polymorphism selected from the group consisting of P #1, P #12, P #15-P #17, P #24, P #41, P #63, P #64, P #69-P #71, P #74-P #79, P #83, P #85-P #87, P #89-P #93, P #97-P #103, P #112-P #118, P #120, P #121, P #123-P #135, P #138-P #141, P #148, P #153-P #168, P #218, P #220-P #226, P #249, P #250, P #253-P #260, P #267, P #495, P #499, P #501, P #503-P #514, P #517-P #539, P #541, P #542, P #544-P #547, P #554-P #585, P #590-P #592, P #595-P #597, P #599-P #614, P #617-P #621, P #623-P #652, P #654-P #669, P #672-P #674, P #676-P #678, P #681, P #682, P #688-P #744, P #746-P #769, P #771-P #813, P #815, P #818-P #824, P #826-P #831 and P #872 (C6:0), preferably the at least one allele represents a “fat allele” for C6:0. In another embodiment according to the alternative aspect of the present invention, the at least one allele is an allele of at least one polymorphism selected from the group consisting of P #1, P #10-P #12, P #16-P #19, P #40-P #45, P #48, P #49, P #51, P #56-P #60, P #63-P #66, P #68-P #93, P #97, P #99-P #101, P #103-P #108, P #111-P #118, P #120, P #121, P #123-P #218, P #220, P #224-P #231, P #254, P #258, P #259, P #268, P #270, P #285-P #288, P #302, P #495, P #496, P #498-P #539, P #541-P #551, P #554-P #586, P #590-P #594, P #628, P #682 and P #683 (C8:0), preferably the at least one allele represents a “fat allele” for C8:0. In another embodiment according to the alternative aspect of the present invention, the at least one allele is an allele of at least one polymorphism selected from the group consisting of P #3, P #8-P #10, P #16-P #20, P #49-P #51, P #56-P #62, P #64, P #69-P #72, P #74, P #76-P #78, P #80-P #96, P #104-P #109, P #111-P #115, P #117, P #118, P #120, P #121, P #127, P #128, P #132, P #133, P #136-P #142, P #148-P #153, P #156-P #166, P #224-P #226, P #285-P #288, P #297-P #302, P #490-P #494, P #501-P #514, P #517-P #539, P #541-P #547, P #551, P #554-P #580, P #585, P #586, P #589-P #593, P #628, P #889-P #891, P #899, P #900, P #902-P #916 (C10:0), preferably the at least one allele represents a “fat allele” for C10:0. In another embodiment according to the alternative aspect of the present invention, the at least one allele is an allele of at least one polymorphism selected from the group consisting of P #3, P #9, P #10, P #16-P #20, P #49-P #53, P #61, P #62, P #64, P #67, P #69-P #72, P #74, P #83, P #85-P #87, P #89-P #96, P #104-P #113, P #115, P #117-P #122, P #127, P #128, P #132, P #133, P #138, P #139, P #141, P #142, P #148-P #151, P #153, P #219, P #224-P #226, P #276, P #285-P #288, P #290-P #302, P #487-P #495, P #497, P #501-P #515, P #517-P #547, P #551, P #554-P #580, P #585, P #586, P #588-P #594, P #887-P #891, P #893-P #916 (C12:0), preferably the at least one allele represents a “fat allele” for C12:0. In another embodiment according to the alternative aspect of the present invention, the at least one allele is an allele of at least one polymorphism selected from the group consisting of P #9, P #10, P #17-P #19, P #34, P #69, P #70, P #83, P #85, P #117, P #120, P #121, P #127, P #128, P #132, P #133, P #139, P #141, P #148-P #151, P #153, P #224-P #226, P #275, P #276, P #279-P #281, P #284-P #302, P #483-P #494, P #497, P #501-P #514, P #517-P #539, P #541, P #543-P #580, P #584-P #594, P #889-P #892, P #899, P #900, P #902-P #916 (C14:0), preferably the at least one allele represents a “fat allele” for C14:0.

In another embodiment according to the alternative aspect of the present invention, the at least one allele is an allele of at least one polymorphism selected from the group consisting of P #243-P #246, P #314-P #332, P #334-P #340, P #346-P #352, P #354-P #363, P #366-P #392, P #394-P #396, P #398-P #410, P #412-P #475 and P #477-P #481 (C14:1 cis-9), preferably the at least one allele represents a “fat allele” for C14:1 cis-9.

In another embodiment according to the alternative aspect of the present invention, altered milk fatty acid composition is decreased amount of C16:0 in milk; and/or increased amount of C18:1 in milk; and/or increased amount of C14:1 cis-9 in milk; and/or increased amount of one or more fatty acids selected from the group consisting of C6:0, C8:0, C10:0, C12:0 and C14:0 in milk; and/or optionally any combination thereof.

According to other particular embodiments, the at least one allele is a “fat allele” or a “non-fat allele”, preferably the latter, for C16:0 of at least one polymorphism selected from the group consisting of P #33, P #241-P #248, P #303-P #312, P #314-P #344, P #346-P #475 and, P #477-P #481.

According to other particular embodiments, the at least one allele is a “non-fat allele” or a “fat allele”, preferably the latter, for C18:1 of at least one polymorphism selected from the group consisting of P #19, P #34, P #38, P #39, P #141, P #148, P #153, P #233-P #240, P #242-P #246, P #271-P #278, P #280-P #283, P #285, P #289-P #302, P #311-P #334, P #339, P #340, P #343-P #475, P #477-P #481, P #484-P #494, P #497, P #543, P #588, P #589, P #916, P #911, P #14, P #37, P #903 and P #7.

According to other particular embodiments, the at least one allele is a “non-fat allele” or a “fat allele”, preferably the latter, for C6:0 of at least one polymorphism selected from the group consisting of P #1, P #12, P #15-P #17, P #24, P #41, P #63, P #64, P #69-P #71, P #74-P #79, P #83, P #85-P #87, P #89-P #93, P #97-P #103, P #112-P #118, P #120, P #121, P #123-P #135, P #138-P #141, P #148, P #153-P #168, P #218, P #220-P #226, P #249, P #250, P #253-P #260, P #267, P #495, P #499, P #501, P #503-P #514, P #517-P #539, P #541, P #542, P #544-P #547, P #554-P #585, P #590-P #592, P #595-P #597, P #599-P #614, P #617-P #621, P #623-P #652, P #654-P #669, P #672-P #674, P #676-P #678, P #681, P #682, P #688-P #744, P #746-P #769, P #771-P #813, P #815, P #818-P #824, P #826-P #831 and P #872.

According to other particular embodiments, the at least one allele is a “non-fat allele” or a “fat allele”, preferably the latter, for C8:0 of at least one polymorphism selected from the group consisting of P #1, P #10-P #12, P #16-P #19, P #40-P #45, P #48, P #49, P #51, P #56-P #60, P #63-P #66, P #68-P #93, P #97, P #99-P #101, P #103-P #108, P #111-P #118, P #120, P #121, P #123-P #218, P #220, P #224-P #231, P #254, P #258, P #259, P #268, P #270, P #285-P #288, P #302, P #495, P #496, P #498-P #539, P #541-P #551, P #554-P #586, P #590-P #594, P #628, P #682 and P #683.

According to other particular embodiments, the at least one allele is a “non-fat allele” or a “fat allele”, preferably the latter, for C10:0 of at least one polymorphism selected from the group consisting of P #3, P #8-P #10, P #16-P #20, P #49-P #51, P #56-P #62, P #64, P #69-P #72, P #74, P #76-P #78, P #80-P #96, P #104-P #109, P #111-P #115, P #117, P #118, P #120, P #121, P #127, P #128, P #132, P #133, P #136-P #142, P #148-P #153, P #156-P #166, P #224-P #226, P #285-P #288, P #297-P #302, P #490-P #494, P #501-P #514, P #517-P #539, P #541-P #547, P #551, P #554-P #580, P #585, P #586, P #589-P #593, P #628, P #889-P #891, P #899, P #900, P #902-P #916.

According to other particular embodiments, the at least one allele is a “non-fat allele” or a “fat allele”, preferably the latter, for C12:0 of at least one polymorphism selected from the group consisting of P #3, P #9, P #10, P #16-P #20, P #49-P #53, P #61, P #62, P #64, P #67, P #69-P #72, P #74, P #83, P #85-P #87, P #89-P #96, P #104-P #113, P #115, P #117-P #122, P #127, P #128, P #132, P #133, P #138, P #139, P #141, P #142, P #148-P #151, P #153, P #219, P #224-P #226, P #276, P #285-P #288, P #290-P #302, P #487-P #495, P #497, P #501-P #515, P #517-P #547, P #551, P #554-P #580, P #585, P #586, P #588-P #594, P #887-P #891, P #893-P #916.

According to other particular embodiments, the at least one allele is a “non-fat allele” or a “fat allele”, preferably the latter, for C14:0 of at least one polymorphism selected from the group consisting of P #9, P #10, P #17-P #19, P #34, P #69, P #70, P #83, P #85, P #117, P #120, P #121, P #127, P #128, P #132, P #133, P #139, P #141, P #148-P #151, P #153, P #224-P #226, P #275, P #276, P #279-P #281, P #284-P #302, P #483-P #494, P #497, P #501-P #514, P #517-P #539, P #541, P #543-P #580, P #584-P #594, P #889-P #892, P #899, P #900, P #902-P #916.

According to other particular embodiments, the at least one allele is a “non-fat allele” or a “fat allele”, preferably the latter, for C14:1 cis-9 of at least one polymorphism selected from the group consisting of P #243-P #246, P #314-P #332, P #334-P #340, P #346-P #352, P #354-P #363, P #366-P #392, P #394-P #396, P #398-P #410, P #412-P #475 and P #477-P #481.

It is to be understood that a “fat-allele” for a specific trait is indicative of increased amount of that trait in milk; and a “non-fat allele” for a specific trait is indicative of decreased amount of that trait in milk (“fat allele”, “non-fat allele” and the respective traits are specified in table 1).

According to other particular embodiments, the present invention provides a method for predicting milk fatty acid composition in a female milk-producing cattle, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 1 to 916, such as SEQ ID NOs: 1 to 310; or at a position corresponding to position 60 of a nucleotide sequence which is derived from any one of SEQ ID NOs: 1 to 916, such as SEQ ID NOs: 1 to 310, by 1 to 30, such as 1 to 20, 1 to 15, 1 to 10, 1 to 5, 1 to 3 or 1 to 2 nucleotide substitutions and/or deletions (preferably substitutions).

In one embodiment according to the present invention, the nucleotide substitution(s) and/or nucleotide deletions (preferably substitutions) are not in the polymorphic site (i.e. position 60) of the derived sequence.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQIDNOs 33, 241-248, 303-312, 314-344, 346-475 and, 477-481 (C16:0); or at a position corresponding to position 60 of a nucleotide sequence which is derived from any one of 33, 241-248, 303-312, 314-344, 346-475 and, 477-481 (C16:0) by 1 to 30, such as 1 to 20, 1 to 15, 1 to 10, 1 to 5, 1 to 3 or 1 to 2 nucleotide substitutions and/or deletions (preferably substitutions).

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 19, 34, 38, 39, 141, 148, 153, 233-240, 242-246, 271-278, 280-283, 285, 289-302, 311-334, 339, 340, 343-475, 477-481, 484-494, 497, 543, 588, 589, 916, 911, 14, 37, 903 and 7 (C18:1); or ata position corresponding to position 60 of a nucleotide sequence which is derived from any one of SEQ ID NOs: 19, 34, 38, 39, 141, 148, 153, 233-240, 242-246, 271-278, 280-283, 285, 289-302, 311-334, 339, 340, 343-475, 477-481, 484-494, 497, 543, 588, 589, 916, 911, 14, 37, 903 and 7 (C18:1) by 1 to 30, such as 1 to 20, 1 to 15, 1 to 10, 1 to 5, 1 to 3 or 1 to 2 nucleotide substitutions and/or deletions (preferably substitutions).

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 243-246, 314-332, 334-340, 346-352, 354-363, 366-392, 394-396, 398-410, 412-475 and 477-481 (C14:1 cis-9); or at a position corresponding to position 60 of a nucleotide sequence which is derived from any one of SEQ ID NOs: 243-246, 314-332, 334-340, 346-352, 354-363, 366-392, 394-396, 398-410, 412-475 and 477-481 (C14:1 cis-9) by 1 to 30, such as 1 to 20, 1 to 15, 1 to 10, 1 to 5, 1 to 3 or 1 to 2 nucleotide substitutions and/or deletions (preferably substitutions).

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 1, 12, 15-17, 24, 41, 63, 64, 69-71, 74-79, 83, 85-87, 89-93, 97-103, 112-118, 120, 121, 123-135, 138-141, 148, 153-168, 218, 220-226, 249, 250, 253-260, 267, 495, 499, 501, 503-514, 517-539, 541, 542, 544-547, 554-585, 590-592, 595-597, 599-614, 617-621, 623-652, 654-669, 672-674, 676-678, 681, 682, 688-744, 746-769, 771-813, 815, 818 824, 826-831 and 872 (C6:0); or at a position corresponding to position 60 of a nucleotide sequence which is derived from any one of SEQ ID NOs: 1, 12, 15-17, 24, 41, 63, 64, 69-71, 74-79, 83, 85-87, 89-93, 97-103, 112-118, 120, 121, 123-135, 138-141, 148, 153-168, 218, 220-226, 249, 250, 253-260, 267, 495, 499, 501, 503-514, 517-539, 541, 542, 544-547, 554-585, 590-592, 595-597, 599-614, 617-621, 623-652, 654-669, 672-674, 676-678, 681, 682, 688-744, 746-769, 771-813, 815, 818-824, 826-831 and 872 (C6:0) by 1 to 30, such as 1 to 20, 1 to 15, 1 to 10, 1 to 5, 1 to 3 or 1 to 2 nucleotide substitutions and/or deletions (preferably substitutions).

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 1, 10-12, 16-19, 40-45, 48, 49, 51, 56-60, 63-66, 68-93, 97, 99-101, 103-108, 111-118, 120, 121, 123-218, 220, 224-231, 254, 258, 259, 268, 270, 285-288, 302, 495, 496, 498-539, 541-551, 554-586, 590-594, 628, 682 and 683 (C8:0); or at a position corresponding to position 60 of a nucleotide sequence which is derived from any one of SEQ ID NOs: 1, 10-12, 16-19, 40-45,48,49,51,56-60, 63-66, 68-93,97,99-101, 103-108, 111-118, 120, 121, 123-218, 220, 224-231, 254, 258, 259, 268, 270, 285-288, 302, 495, 496, 498-539, 541-551, 554-586, 590-594, 628, 682 and 683 (C8:0) by 1 to 30, such as 1 to 20, 1 to 15, 1 to 10, 1 to 5, 1 to 3 or 1 to 2 nucleotide substitutions and/or deletions (preferably substitutions).

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 3, 8-10, 16-20, 49-51, 56-62, 64, 69-72, 74, 76-78, 80-96, 104-109, 111-115, 117, 118, 120, 121, 127, 128, 132, 133, 136-142, 148-153, 156-166, 224-226, 285-288, 297-302, 490-494, 501-514, 517-539, 541-547, 551, 554-580, 585, 586, 589-593, 628, 889-891, 899, 900, 902-916 (C10:0); or at a position corresponding to position 60 of a nucleotide sequence which is derived from any one of SEQ ID NOs: 3, 8-10, 16-20, 49-51, 56-62, 64, 69-72, 74, 76-78, 80-96, 104-109, 111-115, 117, 118, 120, 121, 127, 128, 132, 133, 136-142, 148-153, 156-166, 224-226, 285-288, 297-302, 490-494, 501-514, 517-539, 541-547, 551, 554-580, 585, 586, 589-593, 628, 889-891, 899, 900, 902-916 (C10:0) by 1 to 30, such as 1 to 20, 1 to 15, 1 to 10, 1 to 5, 1 to 3 or 1 to 2 nucleotide substitutions and/or deletions (preferably substitutions).

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 3, 9, 10, 16-20, 49-53, 61, 62, 64, 67, 69-72, 74, 83, 85-87, 89-96, 104-113, 115, 117-122, 127, 128, 132, 133, 138, 139, 141, 142, 148-151, 153, 219, 224-226, 276, 285-288, 290-302, 487-495, 497, 501-515, 517-547, 551, 554-580, 585, 586, 588-594, 887-891, 893-916 (C12:0); or at a position corresponding to position 60 of a nucleotide sequence which is derived from any one of SEQ ID NOs: 3, 9, 10, 16-20, 49-53, 61, 62, 64, 67, 69-72, 74, 83, 85-87, 89-96, 104-113, 115, 117-122, 127, 128, 132, 133, 138, 139, 141, 142, 148-151, 153, 219, 224-226, 276, 285-288, 290-302, 487-495, 497, 501-515, 517-547, 551, 554-580, 585, 586, 588-594, 887-891, 893-916 (C12:0) by 1 to 30, such as 1 to 20, 1 to 15, 1 to 10, 1 to 5, 1 to 3 or 1 to 2 nucleotide substitutions and/or deletions (preferably substitutions).

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 9, 10, 17-19, 34, 69, 70, 83, 85, 117, 120, 121, 127, 128, 132, 133, 139, 141, 148-151, 153, 224-226, 275, 276, 279-281, 284-302, 483-494, 497, 501-514, 517-539, 541, 543-580, 584-594, 889-892, 899, 900, 902-916 (C14:0); or at a position corresponding to position 60 of a nucleotide sequence which is derived from any one of SEQ ID NOs: 9, 10, 17-19, 34, 69, 70, 83, 85, 117, 120, 121, 127, 128, 132, 133, 139, 141, 148-151, 153, 224-226, 275, 276, 279-281, 284-302, 483-494, 497, 501-514, 517-539, 541, 543 580, 584-594, 889-892, 899, 900, 902-916 (C14:0) by 1 to 30, such as 1 to 20, 1 to 15, 1 to 10, 1 to 5, 1 to 3 or 1 to 2 nucleotide substitutions and/or deletions (preferably substitutions).

According to more particular embodiments, the method comprises:

determining the identity of one or more nucleotides of at least one allele, such as two, three or four alleles, of at least one polymorphism, which in a female milk-producing cattle is indicative of altered milk fatty acid composition, within the genome of said female milk-producing cattle, said at least one polymorphism being selected from the group consisting of P #1 to P #916.

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being selected from the group consisting of P #33, P #241-P #248, P #303-P #312, P #314-P #344, P #346-P #475 and, P #477-P #481 (C16:0).

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being selected from the group consisting of P #19, P #34, P #38, P #39, P #141, P #148, P #153, P #233-P #240, P #242-P #246, P #271-P #278, P #280-P #283, P #285, P #289-P #302, P #311-P #334, P #339, P #340, P #343-P #475, P #477-P #481, P #484-P #494, P #497, P #543, P #588, P #589, P #916, P #911, P #14, P #37, P #903 and P #7 (C18:1).

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being selected from the group consisting of of P #243-P #246, P #314-P #332, P #334-P #340, P #346-P #352, P #354-P #363, P #366-P #392, P #394-P #396, P #398-P #410, P #412-P #475 and P #477-P #481 (C14:1 cis-9).

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being selected from the group consisting of P #1, P #12, P #15-P #17, P #24, P #41, P #63, P #64, P #69-P #71, P #74-P #79, P #83, P #85-P #87, P #89-P #93, P #97-P #103, P #112-P #118, P #120, P #121, P #123-P #135, P #138-P #141, P #148, P #153-P #168, P #218, P #220-P #226, P #249, P #250, P #253-P #260, P #267, P #495, P #499, P #501, P #503-P #514, P #517-P #539, P #541, P #542, P #544-P #547, P #554-P #585, P #590-P #592, P #595-P #597, P #599-P #614, P #617-P #621, P #623-P #652, P #654-P #669, P #672-P #674, P #676-P #678, P #681, P #682, P #688-P #744, P #746-P #769, P #771-P #813, P #815, P #818-P #824, P #826-P #831 and P #872 (C6:0).

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being selected from the group consisting of P #1, P #10-P #12, P #16-P #19, P #40-P #45, P #48, P #49, P #51, P #56-P #60, P #63-P #66, P #68-P #93, P #97, P #99-P #101, P #103-P #108, P #111-P #118, P #120, P #121, P #123-P #218, P #220, P #224-P #231, P #254, P #258, P #259, P #268, P #270, P #285-P #288, P #302, P #495, P #496, P #498-P #539, P #541-P #551, P #554-P #586, P #590-P #594, P #628, P #682 and P #683 (C8:0).

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being selected from the group consisting of P #3, P #8-P #10, P #16-P #20, P #49-P #51, P #56-P #62, P #64, P #69-P #72, P #74, P #76-P #78, P #80-P #96, P #104-P #109, P #111-P #115, P #117, P #118, P #120, P #121, P #127, P #128, P #132, P #133, P #136-P #142, P #148-P #153, P #156-P #166, P #224-P #226, P #285-P #288, P #297-P #302, P #490-P #494, P #501-P #514, P #517-P #539, P #541-P #547, P #551, P #554-P #580, P #585, P #586, P #589-P #593, P #628, P #889-P #891, P #899, P #900, P #902-P #916 (C10:0).

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being selected from the group consisting of P #3, P #9, P #10, P #16-P #20, P #49-P #53, P #61, P #62, P #64, P #67, P #69-P #72, P #74, P #83, P #85-P #87, P #89-P #96, P #104-P #113, P #115, P #117-P #122, P #127, P #128, P #132, P #133, P #138, P #139, P #141, P #142, P #148-P #151, P #153, P #219, P #224-P #226, P #276, P #285-P #288, P #290-P #302, P #487-P #495, P #497, P #501-P #515, P #517-P #547, P #551, P #554-P #580, P #585, P #586, P #588-P #594, P #887-P #891, P #893-P #916 (C12:0).

According to more particular embodiments, the method comprises:

determining the identity of one or more nucleotides of at least one allele, such as two, three or four alleles, of at least one polymorphism, which in a female milk-producing cattle is indicative of a desired milk fatty acid composition, within the genome of said cattle, said at least one polymorphism being selected from the group consisting of P #9, P #10, P #17-P #19, P #34, P #69, P #70, P #83, P #85, P #117, P #120, P #121, P #127, P #128, P #132, P #133, P #139, P #141, P #148-P #151, P #153, P #224-P #226, P #275, P #276, P #279-P #281, P #284-P #302, P #483-P #494, P #497, P #501-P #514, P #517-P #539, P #541, P #543-P #580, P #584-P #594, P #889-P #892, P #899, P #900, P #902-P #916 (C14:0).

Having generally described this invention, a further understanding can be obtained by reference to certain specific examples, which are provided herein for purposes of illustration only, and are not intended to be limiting unless otherwise specified.

### EXAMPLES

### Example 1: FTIR Spectroscopy and Variance Component Estimation

Estimation of Bovine Milk Fat Composition from FTIR Spectroscopy Data

Traditionally, detailed milk fat composition has been determined using gas chromatography (GC). This is an accurate but expensive method and, therefore, Fourier transform infrared spectroscopy (FTIR) has become the current standard for routine milk recording.

Liquid milk samples from Norwegian Red (NR) cattle have routinely been analyzed using an FT-IR MilkoScan Combifoss 6500 instrument (Foss, Hillerod, Denmark), and the results recorded with the Regional Laboratories of the Norwegian Herd Recording System. Samples have been homogenized and temperature regulated before entering a cuvette (37 μm) for transmission measurements in the spectral range from 925 cm-1 to 5011 cm-1. The instrument was equipped with a DTGS detector. All spectra were transformed from transmittance to absorbance units. Absorbance spectra were preprocessed by taking the second derivative using a polynomial of degree two and a window size of 9 channels followed by extended multiplicative signal correction (Martens and Stark, 1991) in order to correct for baseline variations and multiplicative effect (Zimmermann and Kohler, 2013).

Recent studies have shown that FT-IR data, calibrated against gas chromatography with flame ionization detector (GC-FID) reference data from the same samples, has the potential to give detailed prediction of milk fat composition (e. g.; Soyeurt et al., 2006; Afseth et al., 2010). An advantage of this approach is that the millions of records obtained by routine recording of cattle can be utilized to estimate genetic parameters and improve traits, such as milk fat composition, by breeding.

To obtain a calibration model for FTIR spectra, approx. 1000 samples obtained from a feeding experiment (Afseth et al., 2010) and from field sampling were analyzed in parallel by FTIR spectroscopy and GC-FID reference analysis. FTIR spectra (regressors) were subsequently calibrated against GC-FID reference values (regressands) by using Powered Partial Least Squares Regression (PPLSR, Indahl, 2005). Calibration was assessed by 20-fold cross-validation, i.e. the calibration data was divided randomly into 20 segments and each of them was used as independent test set at a time. The number of components was selected automatically by estimating if there was a significant improvement of the cross-validated prediction of the regressands when increasing the number of PLS components (linear channel combinations) in the reduced-rank PPLSR model. If improvement of the calibration model was not significant when moving from component number A to component number A-1, A was chosen as the optimal number of components. However, in order to avoid overfitting, maximum number of components was set to 25.

Subsequently, the obtained calibration model was applied to approx. 1,650,000 infrared spectra from the Regional Laboratories of the Norwegian Herd Recording System in the periods February to November 2007 and July 2008 to March 2009.

A total of 24 individual fatty acids and 12 combined traits were calibrated for in the study. Individual fatty acids included seven short- and medium-chained, even-numbered saturated FAs (C4:0, C6:0, C8:0, C10:0, C12:0, C14:0, C16:0), two long-chained saturated FAs (C18:0, C20:0), two odd-numbered saturated FAs (C15:0, C17:0), seven monounsaturated FAs (C14:1 cis-9, C16:1 cis-9, C18:1 cis-9, C18:1 cis-11, C18:1 trans-9, C18:1 trans-10, C18:1 trans-11) and six polyunsaturated FAs (C18:2 cis-9,cis-12, C18:3 cis-9,cis-12,cis-15, arachinonic acid (ARA), conjugated linoleic acid (CLA), docosahexaenoic acid (DHA) and eicosapentaenoic acid (EPA)). The combined traits were CIS (% FAs with cis bonds), TRANS (% FAs with trans bonds), TRANS:CIS (trans:cis ratio), N3 (total amount of omega-3 FAs), N6 (total amount of omega-6 FAs), N3:N6 (omega-3:omega-6 ratio), DNS (de novo FA synthesis, i.e., sum of the short-chained FAs C6:0 to C12:0), SAT (% saturated FAs), MUFA (% monounsaturated FAs), PUFA (% polyunsaturated FAs), TOTAL (total fat yield), and iodine value. NEFA (free fatty acids) and UREA were also included in the genome-wide association analyses, but these traits have built-in prediction equations in the FT-IR instrument and are stored as a routine in the Norwegian Dairy Herd Recording System as parameters of milk quality and feeding, and were therefore not calibrated for in the present study.

Estimation of Genetic Variance Components

The ˜1,650,000 FTIR-based fatty acid (FA) profile predictions for individual cattle (Y) were related to the pedigree structure of the NR population. To condense the information for genetic analyses and remove obvious outliers, a subset of the data was formed. Only FA profiles matching cattle in the herd-recording system were kept. Further, the cattle had to be in 1st to 4th lactation and the test-day between 10 and 320 days after calving. The milk yield at the test-day had to be between 5 and 50 kg, and the fat percentage between 1.75 and 7.0. Finally, the sire had to be an AI bull of NR. Milk samples were recorded on a bimonthly basis. This left 950,170 profiles from 300,126 cattle, with a pedigree of 871,455 animals.

The data was analyzed with the following mixed linear animal repeatability model:

Y=RYMᵢ+RPLⱼ+htdₖ+peₗ+aₘ+eᵢⱼₖₗₘ,

where RYM=fixed effect of region (9 regions) by year and month of the test-day, i=1 to 170. RPL=fixed effect of region by lactation number by 10-day period in lactation of the test-day, j=1 to 1116. htd=random effect of herd by test-day, k=1 to 83,850. pe=random permanent environmental effect of the cattle on her repeated records, 1=1 to 300,126. a=random genetic effect of the animal, m=1 to 871,455. e=random residual effect.

The distributional assumptions for the random effects were htd ˜N(0,Iσ²htd), pe ˜N(0,Iσ²pe), a ˜N(0,Aσ²a) and e ˜N(0,Iσ²e), where 0 is the null vector, I the identity matrix and A is the additive genetic relationship matrix.

The variance components were estimated by the DMU software (Madsen and Jensen, 2007) using an average information algorithm. Given the variance components, breeding value and fixed effects were estimated by the DMU software using iteration on data algorithm.

Results

A key element in this study was to estimate fatty acid composition in milk samples from the nationwide recording based on FTIR spectroscopy data using a GC-FID reference analysis method [11]. The results showed that 29 of the fatty acids, that together constituted more than 90% of the total fat content, achieved cross-validated correlation coefficients above 0.5. These fatty acids where considered predictable and included in further analysis. Mean concentration of these traits from the GC-FID reference analyses as well as cross-validated correlation coefficients and heritabilities are shown in Table 3.

TABLE 3
Mean concentrations from the GC FID reference
analyses, cross validated correlation coefficients
(R²CV) and heritabilities for all traits.

| Trait | Cons | R²CV | h² |
|---|---|---|---|
| C4:0 | 4.16 | 0.73 | 0.353 |
| C6:0 | 2.48 | 0.89 | 0.231 |
| C8:0 | 1.48 | 0.91 | 0.187 |
| C10:0 | 3.2 | 0.91 | 0.171 |
| C12:0 | 3.55 | 0.91 | 0.179 |
| C14:0 | 11.21 | 0.86 | 0.109 |
| C14:1cis-9 | 0.98 | 0.52 | 0.222 |
| C15:0 | 1 | 0.59 | 0.146 |
| C16:0 | 25.25 | 0.77 | 0.145 |
| C16:1cis-9 | 1.17 | 0.51 | 0.146 |
| C17:0 | 0.49 | 0.43 | 0.142 |
| C18:0 | 11.29 | 0.54 | 0.175 |
| C18:1trans-9 | 0.24 | 0.74 | 0.141 |
| C18:1trans-10 | 0.36 | 0.56 | 0.171 |
| C18:1trans-11 | 1.33 | 0.67 | 0.092 |
| C18:1cis-9 | 21.4 | 0.94 | 0.127 |
| C18:1cis-11 | 0.79 | 0.73 | 0.146 |
| C18:2cis-9, cis-12 | 1.39 | 0.61 | 0.172 |
| C18:2cis-9, trans-11 | 0.62 | 0.65 | 0.120 |
| C18:3cis-9, cis-12, cis-15 | 0.54 | 0.42 | 0.190 |
| C20:0 | 0.2 | 0.39 | 0.161 |
| ARA | 0.07 | 0.46 | 0.236 |
| EPA | 0.06 | 0.16 | 0.173 |
| DHA | 0.02 | 0.62 | 0.159 |
| SAT | 64.31 | 0.96 | 0.137 |
| MUFA | 26.28 | 0.96 | 0.130 |
| PUFA | 2.7 | 0.72 | 0.171 |
| CIS | 26.43 | 0.96 | 0.138 |
| TRANS | 2.56 | 0.73 | 0.103 |
| TRANS:CIS | 0.1 | 0.64 | 0.096 |
| DNS | 10.72 | 0.92 | 0.165 |
| N3 | 0.62 | 0.37 | 0.191 |
| N6 | 1.47 | 0.62 | 0.170 |
| N3:N6 | 0.44 | 0.42 | 0.193 |
| TOTAL | 93.29 | 0.59 | 0.106 |

All fatty acids are expressed as percentage by weight of total fatty acid content (on a fatty acid methyl ester basis).

Estimation of variance components showed that relatively high heritabilities were estimated from the FTIR predictions (Table 3). Heritabilities were in general higher for the shorter saturated acids than for the medium length saturated acids and the unsaturated acids.

### Example 2: Genome Wide Association Study (GWAS) and Follow-Up Studies of a Region on BTA13 with Effect on De Novo Synthesized Short-Chained Fatty Acids

Daughter yield deviations (DYD) were derived from the results provided in example 1 as sire averages of daughters' predicted fatty acid compositions. Only traits with an R2CV>0.5 was included in the association study. The study was performed on 991 bulls with phenotypic and genotypic information. The average number of daughters per bull was ˜300.

Genotypes for Genome-Wide Association Analyses

Initial genotyping for the GWAS was performed on 991 Norwegian Red A.I. bulls with phenotypic information using the Affymetrix 25K bovine SNP chip (Affymetrix, Santa Clara, Calif., USA) using standard procedures. SNP filtering reduced the number of useful markers to 17,343. The markers were positioned in the genome using the UMD 3.1 assembly.

Construction of a High Density SNP Dataset with 16,679 SNPs on BTA13

Next, a dense map for fine-mapping on BTA13 was constructed by combining genotypes from the Affymetrix 25K SNP chip with genotypes from the Illumina BovineSNP50 BeadChip (54K) (Illumina, San Diego, Calif., USA) and the Illumina BovineHD Genotyping BeadChip (777K). 1575 NR bulls were genotyped for the 54K chip. 536 of these bulls were also among the 2552 genotyped for the 25K chip. Next, 384 of the 1575 bulls were genotyped for the 777K chip. The three data sets were filtered to remove SNPs with minor allele frequency <0.05 and positioned according to the UMD 3.1 assembly. The 25K dataset was imputed to 54K before the combined 54K dataset was imputed to 777K. All imputations and phasing were performed using BEAGLE v3.3.1 [18] with default options. Phase information of the imputed haplotypes were utilized to identify double recombinants and if possible correct or remove these. The resulting dataset consisted of 1024 NR bulls and 16,679 SNPs on BTA13. Average number of daughters per bull was 278. The 991 bulls used in the previous GWAS step were among these 1024 bulls.

Genome Re-Sequencing and Construction of a Sequence-Level SNP Dataset in the Candidate Gene Region

Whole-genome re-sequencing data were obtained for five NR elite bulls on an Illumina Genome Analyzer GAIIx instrument (Illumina, San Diego, Calif., USA) with 2×108 paired end reads. The five bulls were selected since they had large groups of offspring and were relatively unrelated and therefore represented the genetic diversity of the population. Library preparation was performed using a TruSeq SBS V2-GA kit (Illumina, San Diego, Calif., USA). Adaptor- and quality-trimming of raw reads in FASTQ-format was performed using the FASTX-toolkit v0.0.13 [19]. The reads were aligned against BTA13 (UMD 3.1) using Bowtie v0.12.7 [20] with default parameters. Sorting, marking of PCR duplicates and indexing of the resulting SAM files was done using Samtools v0.1.17 [21]. Between 98.7 and 99.7 percent of the reads were mapped to the bovine reference genome assembly UMD 3.1, including all chromosomes and unplaced scaffolds. Average whole genome sequence coverage for each animal was estimated using total number of sequenced fragments times read length divided by the length of the bovine genome (3 gigabases). Two bulls in the dataset had an average whole genome sequence coverage of about 10×, while three bulls had an average coverage of 4×. Variant calling was performed with Freebayes v0.1.0 [22] with a minimum read coverage of two and a minimum alternate allele count of one. The settings were chosen to maximize calling sensitivity given the relatively low sequence coverage for three of the samples.

Since the settings for the variant calling was set to detect as much variation as possible, the criteria for selecting a novel marker for further genotyping were set rather strict. A total of 1260 markers were found within the two genes NCOA6 and ACSS2 or within 2000 bp on either side of these genes. Of these, all markers in exons and UTRs were selected for genotyping together with intron SNPs that was present in the dbSNP database and co-segregated with the most significant SNP from the analyses of the high density data on BTA13. This approach resulted in 71 markers that were genotyped in 570 animals. However, as expected given the relatively relaxed SNP detection criteria applied initially, several of these markers were found to be monomorphic and hence false positives after genotyping. In total only 17 SNPs passed all steps. Of these, two exonic and 11 intronic SNPs were positioned in NCOA6, one exonic and two intronic SNPs were located in ACSS2, and one SNP was found in the neighboring gene GSS. In order to include missing genotypes, include bulls with trait data that were not genotyped, and to cover also the regions outside the two genes, the 17 novel SNPs were imputed together with SNPs from the BovineHD array positioned in the QTL region using BEAGLE v3.3.1. Hence, the final map consisted of 204 markers situated between 63,488,876 and 65,786,868 bp. Of these, 15 and 9 SNPs were located within NCOA6 and ACSS2, respectively. The total number of bulls with genotype and trait data in the dataset was 782, and the average number of daughters per son was 362. This dataset was used for fine-mapping in the candidate gene region and for haplotype analyses.

Statistical Analyses

A single marker association model was utilized for the GWAS, for the re-sequenced BTA13 map and for the candidate gene map. The analysis was performed for the 29 traits regarded predictable according to the analyses described in example 1, and on preexisting records for urea and NEFA. The model fitted to the performance information for each trait and each SNP was:

DYDᵢ=μ+b+aᵢ+eᵢ

where DYDᵢ is performance of bull i, μ is the overall mean, b is the random effect of the SNP, aᵢ is a random polygenic effect of bull i, and eᵢ is a residual effect. The DYD were weighed by the number of daughters. The genetic and residual variances were estimated from the data. The a were assumed to be from a normal distribution ˜N(0,Aσ²_{A}) where A is the relationship matrix derived from the pedigree, and σ²_{A} is the additive genetic variance. The e were assumed to be from a normal distribution ˜N(0,Wσ²ₑ) where σ²ₑ is the environmental variance.

Since the SNP was coded as a random effect, significance levels were found from the log-likelihoods (log L) of a model containing the SNP effect (Log L(H1)) as well as a model without this SNP effect (Log L(H0)), which were both calculated for each marker using the ASREML package version 2.0 [24]. A Likelihood Ratio Test-statistic (LRT) was calculated as LRT=2*(Log L(H1)−Log L(H0)). Following Baret et al. [25], the distribution of the LRT under the null hypothesis can be seen as a mixture of two chi square distributions with 0 and 1 degrees of freedom, respectively. The significance levels are then found from a chi square distribution with 1 df but doubling the probability levels. Due to the amount of multiple testing performed, we required a rather stringent significance threshold of p=0.00025. The corresponding LRT were thus found from a chi square distribution with 1 df and p=0.0005, thus the LRT must be 12.12 or higher.

Results

A total of 200 significant marker—trait associations were detected. The associations were found on 24 chromosomes and for 32 of the traits. The most interesting results were detected on BTA13, BTA1 and BTA15. P #, SEQIDNO, traits, p-values and allele substitution effects for the most relevant associations are presented in Table 4.

TABLE 4
Most significant associations (i.e., LRT > 12.12) detected
by the genome-wide association analyses. Trait refers to
one or more fatty acids that are significantly associated to
the SNP. Effect is the effect of the SNP on the trait, i.e.,
the difference in concentration of the fatty acid in question
between the two alleles of the SNP, measured as % by
weight of total fat.

| P # | SEQ ID NO | Trait | LRT | Effect |
|---|---|---|---|---|
| 1 | 1 | C8:0 | 12.36 | −0.00802103 |
| | | C6:0 | 12.66 | −0.0105822 |
| 2 | 2 | DHA | 13.8 | 0.000672764 |
| 3 | 3 | DNS | 13.04 | −0.00401878 |
| | | C12:0 | 12.51 | −0.00973376 |
| | | C10:0 | 12.93 | −0.00925379 |
| 4 | 4 | CLA | 13.2 | 0.000416303 |
| 5 | 5 | DHA | 19.66 | 0.000168834 |
| 6 | 6 | CLA | 15.48 | 0.00575959 |
| | | SAT | 13.55 | −0.129466 |
| 7 | 7 | C18:1trans-9 | 15.8 | 0.0024417 |
| 8 | 8 | C10:0 | 12.94 | 0.00463456 |
| | | DNS | 12.31 | 0.00183667 |
| 9 | 9 | DNS | 12.63 | 0.0010928 |
| | | C14:0 | 15.15 | −0.00184979 |
| | | C12:0 | 13.83 | −0.00486508 |
| | | C10:0 | 14.65 | −0.00279912 |
| 10 | 10 | DNS | 17.02 | −0.000256256 |
| | | C8:0 | 16.08 | −0.000256256 |
| | | C14:0 | 13.54 | −0.00037812 |
| | | C12:0 | 15.59 | −0.00142054 |
| | | C10:0 | 17.05 | −0.0011639 |
| 11 | 11 | C8:0 | 12.22 | 0.000683987 |
| 12 | 12 | C8:0 | 16.74 | 0.00128692 |
| | | C6:0 | 14.3 | 0.00477017 |
| 13 | 13 | C4:0 | 18.54 | 0.0143173 |
| 14 | 14 | C18:1cis-11 | 15.62 | 0.000971328 |
| 15 | 15 | C4:0 | 14.44 | −0.00759081 |
| | | C6:0 | 20.66 | −0.00757684 |
| | | CLA | 14.24 | 0.00262246 |
| 16 | 16 | DNS | 16.63 | −0.0138831 |
| | | C8:0 | 21.32 | −0.0138831 |
| | | C6:0 | 14.58 | −0.0144535 |
| | | C10:0 | 14.04 | −0.0367898 |
| 17 | 17 | DNS | 26.24 | −0.0071442 |
| | | C8:0 | 26.98 | −0.0071442 |
| | | C6:0 | 18.76 | −0.00928815 |
| | | C14:0 | 13.16 | −0.0251529 |
| | | C12:0 | 21.97 | −0.0171577 |
| | | C10:0 | 23.69 | −0.0167307 |
| 18 | 18 | DNS | 19.87 | −0.00982001 |
| | | C8:0 | 17.5 | −0.00982001 |
| | | C14:0 | 14.71 | −0.0355515 |
| | | C12:0 | 20.04 | −0.0255966 |
| | | C10:0 | 21.32 | −0.0242391 |
| 19 | 19 | C10:0 | 19.62 | 0.0124673 |
| | | C12:0 | 23.07 | 0.0164781 |
| | | C14:0 | 15.64 | 0.0161827 |
| | | C18:1cis-9 | 14.06 | −0.058246 |
| | | C8:0 | 17.66 | 0.00356834 |
| | | DNS | 25.77 | 0.00356834 |
| 20 | 20 | C12:0 | 15.74 | −0.000167818 |
| | | C10:0 | 14.86 | 0.000867926 |
| 21 | 21 | C4:0 | 18.52 | −0.0129863 |
| 22 | 22 | C4:0 | 14.36 | 0.0169964 |
| 23 | 23 | C4:0 | 12.74 | 0.0168102 |
| 24 | 24 | C6:0 | 12.16 | −0.0036454 |
| | | C4:0 | 20.6 | −0.011403 |
| 25 | 25 | C4:0 | 13 | −0.0181556 |
| 26 | 26 | C4:0 | 14 | 0.0144705 |
| 27 | 27 | C4:0 | 12.7 | 0.0295257 |
| 28 | 28 | C4:0 | 13.5 | 0.0124391 |
| 29 | 29 | C4:0 | 14.48 | −0.0162925 |
| 30 | 30 | C4:0 | 13.74 | 0.0113422 |
| 31 | 31 | C4:0 | 17.6 | 0.031742 |
| 32 | 32 | C4:0 | 12.54 | 0.00846089 |
| 33 | 33 | C16:0 | 12.27 | 0.0413636 |
| 34 | 34 | SAT | 15.56 | −0.0399403 |
| | | MUFA | 13.68 | 0.0342139 |
| | | C18:1cis-9 | 14.45 | 0.0173785 |
| | | C14:0 | 12.12 | −0.0118568 |
| 35 | 35 | SAT | 13.14 | −0.098612 |
| 36 | 36 | NEFA | 14.96 | 0.00103029 |
| 37 | 37 | C18:1cis-11 | 13.14 | −0.000169859 |
| 38 | 38 | SAT | 12.31 | 0.0177589 |
| | | C18:1cis-9 | 12.18 | −0.0100095 |
| 39 | 39 | SAT | 14.2 | 0.0109863 |
| | | MUFA | 13.75 | −0.0181838 |
| | | C18:1cis-9 | 13.12 | −0.0193747 |

The most notable results were detected on BTA13, where a large number of SNPs located between 55.4 and 66.1 Mb were strongly associated to all short- and medium-chained, saturated de novo synthesized milk fatty acids (i.e.; C4:0 to C14:0 and DNS). The most significant marker was situated close to a very likely candidate gene; acyl-CoA synthetase short-chain family member 2 (ACSS2). ACSS2 is encoding an enzyme that catalyzes the activation of acetate for use in de novo synthesis of short-chained fatty acids.

The next steps therefore aimed to fine-map this region and, if possible, to identify the causal DNA variation underlying the variation in de novo synthesis. First, all traits found significant by the initial GWAS was reanalyzed for 16,679 SNPs on a high density map covering the entire length of BTA13. All significant results are shown in Table 5. The results pointed out a nearby gene, nuclear receptor coactivator 6 (NCOA6), as more significant than ACSS2. The putative role of this gene in fatty acid synthesis have so far not been investigated. However, NCOA6 is a ligand for transcription factors such as PPARy, which affects transcription of genes involved in fatty acid transport, and is proposed as a major regulator of bovine milk fat synthesis.

TABLE 5
Significant results (i.e., LRT > 12.12) from the
analyses of de novo-synthesized fatty acids using
a high density marker material on BTA13. Trait
refers to one or more fatty acids that are significantly
associated to the SNP. Effect is the effect of the SNP
on the trait, i.e., the difference in concentration of
the fatty acid in question between the two alleles of
the SNP, measured as % by weight of total fat.

| P# | SEQ ID NO | Trait | LRT | Effect |
|---|---|---|---|---|
| 40 | 40 | C8:0 | 13.4 | 0.00188608 |
| 41 | 41 | C6:0 | 13.2 | 0.00192254 |
| | | C8:0 | 14.8 | 0.000788914 |
| 42 | 42 | C8:0 | 12.4 | 0.000903481 |
| 43 | 43 | C8:0 | 13.3 | −0.000952205 |
| 44 | 44 | C8:0 | 14.6 | 0.00246485 |
| | | DNS | 12.1 | 0.00246485 |
| 45 | 45 | C8:0 | 12.8 | 0.00227257 |
| 46 | 46 | C4:0 | 12.1 | −0.00640506 |
| 15 | 15 | C4:0 | 14.3 | −0.00759081 |
| | | C6:0 | 16 | −0.00757684 |
| 47 | 47 | C4:0 | 12.6 | −0.00687608 |
| 48 | 48 | C8:0 | 12.1 | −0.00293636 |
| 49 | 49 | DNS | 17.5 | −0.00617226 |
| | | C8:0 | 17.5 | −0.00617226 |
| | | C12:0 | 14.4 | −0.0178275 |
| | | C10:0 | 16 | −0.0160308 |
| 50 | 50 | C12:0 | 15.1 | −0.0135792 |
| | | C10:0 | 12.6 | −0.0103807 |
| 51 | 51 | C10:0 | 13.9 | −0.00996145 |
| | | C12:0 | 14.1 | −0.0113767 |
| | | C8:0 | 12.2 | −0.00374251 |
| | | DNS | 15.6 | −0.00374251 |
| 52 | 52 | C12:0 | 12.3 | −0.00769547 |
| 53 | 53 | C12:0 | 12.3 | −0.00769547 |
| 54 | 54 | C4:0 | 13.3 | −0.0098508 |
| 55 | 55 | C4:0 | 12.3 | −0.01215 |
| 56 | 56 | C10:0 | 12.8 | −0.0133895 |
| | | C8:0 | 16.4 | −0.00578424 |
| | | DNS | 14.9 | −0.00578424 |
| 57 | 57 | DNS | 15.3 | −0.00578424 |
| | | C8:0 | 16.9 | −0.00578424 |
| | | C10:0 | 13.2 | −0.0133895 |
| 58 | 58 | C10:0 | 12.1 | −0.0125997 |
| | | C8:0 | 15.6 | −0.005502 |
| | | DNS | 14.1 | −0.005502 |
| 59 | 59 | DNS | 14.9 | −0.00578424 |
| | | C8:0 | 16.4 | −0.00578424 |
| | | C10:0 | 12.8 | −0.0133895 |
| 60 | 60 | C10:0 | 12.6 | −0.0133895 |
| | | C8:0 | 16.1 | −0.00578424 |
| | | DNS | 14.8 | −0.00578424 |
| 61 | 61 | C12:0 | 23.1 | 0.0144813 |
| | | C10:0 | 20.1 | 0.0130699 |
| 62 | 62 | C12:0 | 16.1 | 0.0141153 |
| | | C10:0 | 13.1 | 0.0121343 |
| 63 | 63 | C8:0 | 13.7 | −0.0175384 |
| | | C6:0 | 13.5 | −0.0192056 |
| 64 | 64 | C10:0 | 20.3 | −0.0625506 |
| | | C12:0 | 12.7 | −0.067408 |
| | | C6:0 | 29.7 | −0.0273063 |
| | | C8:0 | 33.4 | −0.0248163 |
| | | DNS | 23.5 | −0.0248163 |
| 65 | 65 | C8:0 | 16.3 | 0.00773878 |
| | | DNS | 12.4 | 0.00773878 |
| 66 | 66 | DNS | 12.4 | 0.00773878 |
| | | C8:0 | 16.3 | 0.00773878 |
| 67 | 67 | C12:0 | 12.9 | 0.0162291 |
| 68 | 68 | C8:0 | 16.3 | 0.00775108 |
| | | DNS | 12.4 | 0.00775108 |
| 69 | 69 | DNS | 30.2 | −0.0198406 |
| | | C8:0 | 41.7 | −0.0198406 |
| | | C6:0 | 31.1 | −0.0213763 |
| | | C12:0 | 17.8 | −0.0532377 |
| | | C10:0 | 26.5 | −0.0495702 |
| 70 | 70 | C10:0 | 26.5 | −0.0495702 |
| | | C12:0 | 17.8 | −0.0532377 |
| | | C6:0 | 31.1 | −0.0213763 |
| | | C8:0 | 41.7 | −0.0198406 |
| | | DNS | 30.2 | −0.0198406 |
| 71 | 71 | DNS | 16.9 | −0.00905516 |
| | | C8:0 | 18.8 | −0.00905516 |
| | | C6:0 | 12.3 | −0.0115379 |
| | | C12:0 | 12.8 | −0.0203055 |
| | | C10:0 | 16 | −0.0205959 |
| 72 | 72 | DNS | 17.2 | −0.00884674 |
| | | C8:0 | 18.8 | −0.00884674 |
| | | C12:0 | 13.2 | −0.019146 |
| | | C10:0 | 16.5 | −0.0197048 |
| 73 | 73 | C8:0 | 17.1 | −0.00840382 |
| | | DNS | 12.9 | −0.00840382 |
| 74 | 74 | DNS | 19.1 | −0.0079169 |
| | | C8:0 | 20.3 | −0.0079169 |
| | | C6:0 | 13.8 | −0.0107992 |
| | | C12:0 | 15.1 | −0.0161472 |
| | | C10:0 | 17.2 | −0.0167854 |
| 75 | 75 | C6:0 | 24.3 | −0.0207355 |
| | | C8:0 | 25 | −0.0179043 |
| | | DNS | 15.9 | −0.0179043 |
| 76 | 76 | DNS | 17.1 | −0.0178593 |
| | | C8:0 | 27 | −0.0178593 |
| | | C6:0 | 24.6 | −0.020456 |
| | | C10:0 | 13.2 | −0.0446094 |
| 77 | 77 | C10:0 | 12.8 | −0.0185742 |
| | | C6:0 | 12.6 | −0.00918383 |
| | | C8:0 | 14.3 | −0.00741921 |
| | | DNS | 12.7 | −0.00741921 |
| 78 | 78 | DNS | 12.7 | −0.00727749 |
| | | C8:0 | 14.3 | −0.00727749 |
| | | C6:0 | 12.6 | −0.0090346 |
| | | C10:0 | 12.8 | −0.0181508 |
| 79 | 79 | C6:0 | 12.3 | −0.00879036 |
| | | C8:0 | 13.8 | −0.00720897 |
| 80 | 80 | C8:0 | 14.6 | 0.00414185 |
| | | C10:0 | 13.8 | 0.0150976 |
| 81 | 81 | C8:0 | 14.6 | 0.00450224 |
| | | C10:0 | 13.8 | 0.0154715 |
| 82 | 82 | C10:0 | 13.2 | 0.0154715 |
| | | C8:0 | 14.2 | 0.00450224 |
| 83 | 83 | C10:0 | 23.5 | 0.0283554 |
| | | C12:0 | 19 | 0.0311255 |
| | | C6:0 | 19.6 | 0.0100658 |
| | | C8:0 | 27.8 | 0.0101171 |
| | | DNS | 22.8 | 0.0101171 |
| 84 | 84 | C10:0 | 13.2 | 0.0154715 |
| | | C8:0 | 14.2 | 0.00450224 |
| 85 | 85 | DNS | 22.8 | 0.0102997 |
| | | C8:0 | 27.8 | 0.0102997 |
| | | C6:0 | 19.6 | 0.0104809 |
| | | C12:0 | 19 | 0.0315693 |
| | | C10:0 | 23.5 | 0.0286623 |
| 86 | 86 | C10:0 | 27.1 | 0.0217681 |
| | | C12:0 | 27.2 | 0.0260882 |
| | | C6:0 | 12.6 | 0.00443943 |
| | | C8:0 | 23.1 | 0.00630516 |
| | | DNS | 24.8 | 0.00630516 |
| 87 | 87 | DNS | 24.8 | 0.00662192 |
| | | C8:0 | 23.1 | 0.00662192 |
| | | C6:0 | 12.6 | 0.00478141 |
| | | C12:0 | 27.2 | 0.0273646 |
| | | C10:0 | 27.1 | 0.0227998 |
| 88 | 88 | C10:0 | 13.2 | 0.0154715 |
| | | C8:0 | 14.2 | 0.00450224 |
| 89 | 89 | C10:0 | 28 | 0.0222747 |
| | | C12:0 | 27.8 | 0.02671 |
| | | C6:0 | 13.4 | 0.0045743 |
| | | C8:0 | 24 | 0.00645377 |
| | | DNS | 25.7 | 0.00645377 |
| 90 | 90 | C10:0 | 28 | 0.0221045 |
| | | C12:0 | 27.8 | 0.0264912 |
| | | C6:0 | 13.4 | 0.00450331 |
| | | C8:0 | 24 | 0.00639396 |
| | | DNS | 25.7 | 0.00639396 |
| 91 | 91 | C10:0 | 28 | 0.0222226 |
| | | C12:0 | 27.8 | 0.0267306 |
| | | C6:0 | 13.4 | 0.00439091 |
| | | C8:0 | 24 | 0.00638839 |
| | | DNS | 25.7 | 0.00638839 |
| 92 | 92 | DNS | 25.7 | 0.00645377 |
| | | C8:0 | 24 | 0.00645377 |
| | | C6:0 | 13.4 | 0.0045743 |
| | | C12:0 | 27.8 | 0.02671 |
| | | C10:0 | 28 | 0.0222747 |
| 93 | 93 | C10:0 | 28.7 | 0.0222747 |
| | | C12:0 | 28.6 | 0.02671 |
| | | C6:0 | 13.8 | 0.0045743 |
| | | C8:0 | 24.9 | 0.00645377 |
| | | DNS | 26.5 | 0.00645377 |
| 94 | 94 | C12:0 | 18.6 | 0.0266584 |
| | | C10:0 | 16.9 | 0.0221645 |
| 95 | 95 | C10:0 | 16.9 | 0.0221645 |
| | | C12:0 | 18.6 | 0.0266584 |
| 96 | 96 | C12:0 | 19.6 | 0.0266583 |
| | | C10:0 | 17.8 | 0.0221645 |
| 97 | 97 | C8:0 | 16.4 | 0.00806355 |
| | | C6:0 | 16.5 | 0.0101885 |
| 98 | 98 | C6:0 | 14.3 | −0.0112651 |
| 99 | 99 | C6:0 | 15.1 | −0.0126808 |
| | | C8:0 | 14.2 | −0.0105791 |
| 100 | 100 | C6:0 | 16.5 | −0.0126917 |
| | | C8:0 | 14.3 | −0.0110808 |
| 101 | 101 | C8:0 | 14.3 | −0.0109968 |
| | | C6:0 | 16.5 | −0.0125584 |
| 102 | 102 | C6:0 | 12.1 | −0.0134234 |
| 103 | 103 | C6:0 | 14.4 | −0.0125358 |
| | | C8:0 | 12.8 | −0.0108727 |
| 104 | 104 | DNS | 14.3 | 0.00787918 |
| | | C8:0 | 14.2 | 0.00787918 |
| | | C12:0 | 15 | 0.0278349 |
| | | C10:0 | 13.9 | 0.024037 |
| 105 | 105 | DNS | 14.3 | 0.00787918 |
| | | C8:0 | 14.2 | 0.00787918 |
| | | C12:0 | 15 | 0.0278349 |
| | | C10:0 | 13.9 | 0.024037 |
| 106 | 106 | DNS | 14.3 | 0.00787918 |
| | | C8:0 | 14.2 | 0.00787918 |
| | | C12:0 | 15 | 0.0278349 |
| | | C10:0 | 13.9 | 0.024037 |
| 107 | 107 | DNS | 14.3 | 0.00787918 |
| | | C8:0 | 14.2 | 0.00787918 |
| | | C12:0 | 15 | 0.0278349 |
| | | C10:0 | 13.9 | 0.024037 |
| 108 | 108 | C10:0 | 13.9 | 0.024037 |
| | | C12:0 | 15 | 0.0278349 |
| | | C8:0 | 14.2 | 0.00787918 |
| | | DNS | 14.3 | 0.00787918 |
| 109 | 109 | C12:0 | 13.9 | 0.0197669 |
| | | C10:0 | 12.6 | 0.0177329 |
| 110 | 110 | C12:0 | 13.1 | 0.0197669 |
| 111 | 111 | DNS | 15.5 | 0.00564728 |
| | | C8:0 | 13 | 0.00564728 |
| | | C12:0 | 16.1 | 0.0190808 |
| | | C10:0 | 15.7 | 0.0168402 |
| 112 | 112 | DNS | 18.9 | −0.0105537 |
| | | C8:0 | 23.6 | −0.0105537 |
| | | C6:0 | 17.9 | −0.0117802 |
| | | C12:0 | 13.8 | −0.0284888 |
| | | C10:0 | 18.9 | −0.0273379 |
| 113 | 113 | C10:0 | 17.7 | −0.0273379 |
| | | C12:0 | 12.9 | −0.0284888 |
| | | C6:0 | 17.3 | −0.0117802 |
| | | C8:0 | 22.4 | −0.0105537 |
| | | DNS | 17.7 | −0.0105537 |
| 114 | 114 | DNS | 14.8 | −0.0106052 |
| | | C8:0 | 18 | −0.0106052 |
| | | C6:0 | 14.8 | −0.0126255 |
| | | C10:0 | 13.8 | −0.0264331 |
| 115 | 115 | DNS | 20.2 | −0.0103952 |
| | | C8:0 | 24.4 | −0.0103952 |
| | | C6:0 | 18.3 | −0.0118818 |
| | | C12:0 | 14.9 | −0.0278326 |
| | | C10:0 | 20.3 | −0.0270128 |
| 116 | 116 | C8:0 | 12.1 | 0.00862018 |
| | | C6:0 | 13.7 | 0.0121927 |
| 117 | 117 | DNS | 33.5 | −0.0284217 |
| | | C8:0 | 49.7 | −0.0284217 |
| | | C6:0 | 39 | −0.0294283 |
| | | C12:0 | 19.7 | −0.0817469 |
| | | C10:0 | 29.6 | −0.0745318 |
| 118 | 118 | C10:0 | 24.3 | −0.0256072 |
| | | C12:0 | 23.9 | −0.030413 |
| | | C6:0 | 16.4 | −0.00703065 |
| | | C8:0 | 26.1 | −0.0083195 |
| | | DNS | 25.5 | −0.0083195 |
| 119 | 119 | C12:0 | 13.2 | 0.0147947 |
| 120 | 120 | C10:0 | 24.2 | −0.0310331 |
| | | C12:0 | 19.7 | −0.0332942 |
| | | C6:0 | 24 | −0.0146789 |
| | | C8:0 | 31.5 | −0.0122436 |
| | | DNS | 25.8 | −0.0122436 |
| 121 | 121 | DNS | 25.8 | −0.0122114 |
| | | C8:0 | 31.5 | −0.0122114 |
| | | C6:0 | 24 | −0.0146497 |
| | | C12:0 | 19.7 | −0.0330648 |
| | | C10:0 | 24.2 | −0.0309041 |
| 122 | 122 | C12:0 | 13.3 | 0.036836 |
| 123 | 123 | C6:0 | 18.7 | −0.0102934 |
| | | C8:0 | 16.7 | −0.00870533 |
| 124 | 124 | C6:0 | 16.1 | 0.0106208 |
| | | C8:0 | 15.9 | 0.00682733 |
| 125 | 125 | C8:0 | 15.9 | 0.0068272 |
| | | C6:0 | 16.1 | 0.0106206 |
| 126 | 126 | C8:0 | 15.9 | 0.0068272 |
| | | C6:0 | 16.1 | 0.0106206 |
| 127 | 127 | C10:0 | 12.2 | −0.0271098 |
| | | C6:0 | 16.2 | −0.0118919 |
| | | C8:0 | 20.1 | −0.0106605 |
| | | DNS | 14.6 | −0.0106605 |
| 128 | 128 | DNS | 18.1 | −0.00893882 |
| | | C8:0 | 22 | −0.00893882 |
| | | C6:0 | 16.1 | −0.00997547 |
| | | C12:0 | 12.9 | −0.0256983 |
| | | C10:0 | 15.2 | −0.0232924 |
| 129 | 129 | C8:0 | 13 | −0.0118772 |
| 130 | 130 | C8:0 | 13 | −0.0118772 |
| 131 | 131 | C8:0 | 13 | −0.0118772 |
| 132 | 132 | C10:0 | 16 | −0.0383403 |
| | | C6:0 | 25 | −0.0173749 |
| | | C8:0 | 28.6 | −0.0155207 |
| | | DNS | 18.5 | −0.0155207 |
| 133 | 133 | C10:0 | 16.3 | −0.0322227 |
| | | C6:0 | 27.5 | −0.0154619 |
| | | C8:0 | 30 | −0.013215 |
| | | DNS | 19.7 | −0.013215 |
| 134 | 134 | C8:0 | 13 | −0.0118772 |
| 135 | 135 | C8:0 | 13 | −0.0118772 |
| 136 | 136 | DNS | 13.5 | −0.0101673 |
| | | C8:0 | 17.2 | −0.0101673 |
| | | C10:0 | 13 | −0.0256289 |
| 137 | 137 | DNS | 13.6 | −0.0101604 |
| | | C8:0 | 17.5 | −0.0101604 |
| | | C10:0 | 13.2 | −0.0254864 |
| 138 | 138 | DNS | 18.4 | −0.0138838 |
| | | C8:0 | 23.2 | −0.0138838 |
| | | C6:0 | 13.6 | −0.0144543 |
| | | C12:0 | 13.8 | −0.04023 |
| | | C10:0 | 17.7 | −0.0367916 |
| 16 | 16 | DNS | 20.6 | −0.0138831 |
| | | C8:0 | 25.2 | −0.0138831 |
| | | C6:0 | 13.6 | −0.0144535 |
| | | C12:0 | 15.4 | −0.040228 |
| | | C10:0 | 19.5 | −0.0367898 |
| 139 | 139 | C10:0 | 23.7 | 0.028243 |
| | | C12:0 | 24.5 | 0.0335376 |
| | | C6:0 | 12.2 | 0.00718847 |
| | | C8:0 | 24.7 | 0.00897561 |
| | | DNS | 25.8 | 0.00897561 |
| 140 | 140 | DNS | 21.7 | −0.0119712 |
| | | C8:0 | 33.4 | −0.0119712 |
| | | C6:0 | 27.2 | −0.013484 |
| | | C10:0 | 16.8 | −0.0294542 |
| 141 | 141 | C10:0 | 43.6 | −0.0389109 |
| | | C12:0 | 35.1 | −0.0415682 |
| | | C6:0 | 46.5 | −0.0176825 |
| | | C8:0 | 62.6 | −0.0154576 |
| | | DNS | 48.8 | −0.0154576 |
| 142 | 142 | DNS | 13 | −0.00761768 |
| | | C8:0 | 12.3 | −0.00761768 |
| | | C12:0 | 12.5 | −0.0193179 |
| | | C10:0 | 13 | −0.0188101 |
| 143 | 143 | DNS | 12.2 | −0.00711438 |
| | | C8:0 | 14 | −0.00711438 |
| 144 | 144 | C8:0 | 14.5 | −0.0071442 |
| | | DNS | 12.7 | −0.0071442 |
| 145 | 145 | C8:0 | 14.5 | −0.0071442 |
| | | DNS | 12.7 | −0.0071442 |
| 146 | 146 | DNS | 13.1 | −0.00712916 |
| | | C8:0 | 15 | −0.00712916 |
| 17 | 17 | DNS | 12.6 | −0.0071442 |
| | | C8:0 | 14.5 | −0.0071442 |
| 147 | 147 | DNS | 12.7 | −0.00714426 |
| | | C8:0 | 14.5 | −0.00714426 |
| 148 | 148 | DNS | 26.4 | −0.0140383 |
| | | C8:0 | 29.6 | −0.0140383 |
| | | C6:0 | 19.7 | −0.015477 |
| | | C12:0 | 19.8 | −0.0437927 |
| | | C10:0 | 24.2 | −0.0394483 |
| 149 | 149 | C10:0 | 14.9 | −0.0166395 |
| | | C12:0 | 14.8 | −0.0186586 |
| 150 | 150 | C12:0 | 14 | −0.0186586 |
| | | C10:0 | 14.1 | −0.0166395 |
| 151 | 151 | C10:0 | 12.2 | −0.0240498 |
| | | C8:0 | 12.2 | −0.00897534 |
| 152 | 152 | C8:0 | 12.4 | −0.00899286 |
| | | C10:0 | 12.4 | −0.0240665 |
| 153 | 153 | DNS | 21.7 | −0.0108675 |
| | | C8:0 | 24.1 | −0.0108675 |
| | | C6:0 | 15.6 | −0.0129423 |
| | | C12:0 | 17.5 | −0.0283391 |
| | | C10:0 | 20.5 | −0.0270795 |
| 18 | 18 | DNS | 15.6 | −0.00982001 |
| | | C8:0 | 18.6 | −0.00982001 |
| | | C12:0 | 12.5 | −0.0255966 |
| | | C10:0 | 14.2 | −0.0242391 |
| 154 | 154 | C8:0 | 17.5 | −0.0163058 |
| | | C6:0 | 17.5 | −0.0195499 |
| 155 | 155 | C6:0 | 13.2 | −0.00577609 |
| | | C8:0 | 13.9 | −0.00605426 |
| 156 | 156 | C10:0 | 15.8 | −0.0576564 |
| | | C6:0 | 30.7 | −0.0234211 |
| | | C8:0 | 31.3 | −0.0222813 |
| | | DNS | 19.9 | −0.0222813 |
| 157 | 157 | DNS | 15 | −0.0105046 |
| | | C8:0 | 21.3 | −0.0105046 |
| | | C6:0 | 16.3 | −0.0108847 |
| | | C10:0 | 13.1 | −0.0289462 |
| 158 | 158 | C10:0 | 13.1 | −0.0289462 |
| | | C6:0 | 16.3 | −0.0108847 |
| | | C8:0 | 21.3 | −0.0105046 |
| | | DNS | 15 | −0.0105046 |
| 159 | 159 | DNS | 15 | −0.0105046 |
| | | C8:0 | 21.3 | −0.0105046 |
| | | C6:0 | 16.3 | −0.0108847 |
| | | C10:0 | 13.1 | −0.0289462 |
| 160 | 160 | DNS | 15 | −0.0105046 |
| | | C8:0 | 21.3 | −0.0105046 |
| | | C6:0 | 16.3 | −0.0108847 |
| | | C10:0 | 13.1 | −0.0289462 |
| 161 | 161 | DNS | 15 | −0.0105046 |
| | | C8:0 | 21.3 | −0.0105046 |
| | | C6:0 | 16.3 | −0.0108847 |
| | | C10:0 | 13.1 | −0.0289462 |
| 162 | 162 | C10:0 | 13.1 | −0.0289462 |
| | | C6:0 | 16.3 | −0.0108847 |
| | | C8:0 | 21.3 | −0.0105046 |
| | | DNS | 15 | −0.0105046 |
| 163 | 163 | C10:0 | 13.1 | −0.0282738 |
| | | C6:0 | 16.3 | −0.0104727 |
| | | C8:0 | 21.3 | −0.010219 |
| | | DNS | 15 | −0.010219 |
| 164 | 164 | DNS | 18.1 | −0.0222813 |
| | | C8:0 | 29.6 | −0.0222813 |
| | | C6:0 | 30.3 | −0.0234211 |
| | | C10:0 | 13.5 | −0.0576564 |
| 165 | 165 | DNS | 18.1 | −0.0222813 |
| | | C8:0 | 29.6 | −0.0222813 |
| | | C6:0 | 30.3 | −0.0234211 |
| | | C10:0 | 13.5 | −0.0576564 |
| 166 | 166 | DNS | 19.9 | −0.0222813 |
| | | C8:0 | 31.3 | −0.0222813 |
| | | C6:0 | 30.7 | −0.0234211 |
| | | C10:0 | 15.8 | −0.0576564 |
| 167 | 167 | C6:0 | 13.5 | 0.00620273 |
| | | C8:0 | 16.7 | 0.00722304 |
| | | DNS | 12.4 | 0.00722304 |
| 168 | 168 | C8:0 | 12.9 | −0.0124996 |
| | | C6:0 | 13.3 | −0.0149711 |
| 169 | 169 | C8:0 | 14.8 | 0.0103145 |
| 170 | 170 | DNS | 12.1 | 0.0103145 |
| | | C8:0 | 15.1 | 0.0103145 |
| 171 | 171 | C8:0 | 15 | 0.0103951 |
| 172 | 172 | C8:0 | 15 | 0.0103951 |
| 173 | 173 | C8:0 | 15 | 0.0104669 |
| 174 | 174 | C8:0 | 15 | 0.0103951 |
| 175 | 175 | C8:0 | 15 | 0.0103282 |
| 176 | 176 | C8:0 | 15 | 0.0103282 |
| 177 | 177 | C8:0 | 15.7 | 0.0104445 |
| | | DNS | 12.3 | 0.0104445 |
| 178 | 178 | C8:0 | 15 | 0.0105151 |
| 179 | 179 | C8:0 | 12.1 | 0.0093436 |
| 180 | 180 | C8:0 | 15.1 | 0.0102479 |
| | | DNS | 12.1 | 0.0102479 |
| 181 | 181 | C8:0 | 15.2 | 0.0103358 |
| | | DNS | 12.1 | 0.0103358 |
| 182 | 182 | C8:0 | 14.3 | 0.0103198 |
| 183 | 183 | C8:0 | 14.2 | 0.0110059 |
| 184 | 184 | C8:0 | 14.2 | 0.0110059 |
| 185 | 185 | C8:0 | 14.2 | 0.0110059 |
| 186 | 186 | C8:0 | 14.2 | 0.0100395 |
| 187 | 187 | C8:0 | 14.2 | 0.0100946 |
| 188 | 188 | C8:0 | 14.2 | 0.0100946 |
| 189 | 189 | C8:0 | 14.2 | 0.0100946 |
| 190 | 190 | C8:0 | 14.2 | 0.0100946 |
| 191 | 191 | C8:0 | 14.2 | 0.0100946 |
| 192 | 192 | C8:0 | 14.2 | 0.0101953 |
| 193 | 193 | C8:0 | 14.2 | 0.0100946 |
| 194 | 194 | C8:0 | 14.2 | 0.0100508 |
| 195 | 195 | C8:0 | 14.2 | 0.0100946 |
| 196 | 196 | C8:0 | 15.2 | 0.0099443 |
| 197 | 197 | C8:0 | 14 | 0.0100946 |
| 198 | 198 | C8:0 | 14.2 | 0.0102796 |
| 199 | 199 | C8:0 | 14.2 | 0.0100946 |
| 200 | 200 | C8:0 | 14.2 | 0.0100946 |
| 201 | 201 | C8:0 | 14.2 | 0.0100946 |
| 202 | 202 | C8:0 | 14.2 | 0.0102213 |
| 203 | 203 | C8:0 | 14.2 | 0.0102215 |
| 204 | 204 | C8:0 | 13.9 | 0.00960664 |
| 205 | 205 | C8:0 | 13.9 | 0.00960664 |
| 206 | 206 | C8:0 | 13.9 | 0.00973167 |
| 207 | 207 | C8:0 | 12.7 | 0.0102215 |
| 208 | 208 | C8:0 | 12.5 | 0.0102215 |
| 209 | 209 | C8:0 | 12.1 | 0.0102213 |
| 210 | 210 | C8:0 | 12.6 | 0.0102284 |
| 211 | 211 | C8:0 | 12.5 | 0.0102201 |
| 212 | 212 | C8:0 | 13.3 | 0.00597043 |
| 213 | 213 | C8:0 | 13 | 0.0059564 |
| 214 | 214 | C8:0 | 12.9 | 0.00487396 |
| 215 | 215 | C8:0 | 12.9 | 0.00626975 |
| 216 | 216 | C8:0 | 13 | 0.00615764 |
| 217 | 217 | C8:0 | 12.6 | 0.00642756 |
| 218 | 218 | C6:0 | 13.7 | −0.0117201 |
| | | C8:0 | 13.5 | −0.0105932 |
| 219 | 219 | C12:0 | 12.8 | 0.0163537 |
| 220 | 220 | C6:0 | 15 | −0.00181288 |
| | | C8:0 | 12.1 | −0.00261333 |
| 221 | 221 | C6:0 | 14.7 | −0.00455885 |
| 222 | 222 | C6:0 | 14.7 | −0.00360259 |
| 223 | 223 | C6:0 | 14.7 | −0.00413304 |

In the final step, all existing variations in a region covering NCOA6 and ACSS2 was aimed identified, and selected markers were genotyped and reanalyzed for the relevant traits. Again, the most significant associations were detected for markers within NCOA6, and none of the ACSS2 SNPs were among the 20 most significant for any of the traits. All significant results are shown in table 6.

TABLE 6
Significant results (i.e., LRT > 12.12) from the analyses of de
novo-synthesized fatty acids for markers in the candidate gene region.
Trait refers to one or more fatty acids that are significantly associated
to the SNP. Effect is the effect of the SNP on the trait, i.e., the
difference in concentration of the fatty acid in question between the
two alleles of the SNP, measured as % by weight of total fat.

| P# | SEQIDNO | Trait | LRT | Effect |
|---|---|---|---|---|
| 120 | 120 | C10:0 | 25.12 | −0.0310331 |
| | | C12:0 | 20.66 | −0.0332942 |
| | | C14:0 | 17.96 | −0.0490037 |
| | | C6:0 | 25.02 | −0.0146789 |
| | | C8:0 | 32.08 | −0.0122436 |
| | | DNS | 26.49 | −0.0122436 |
| 121 | 121 | C10:0 | 25.12 | −0.0309041 |
| | | C12:0 | 20.66 | −0.0330648 |
| | | C14:0 | 17.96 | −0.0486681 |
| | | C6:0 | 25.02 | −0.0146497 |
| | | C8:0 | 32.08 | −0.0122114 |
| | | DNS | 26.49 | −0.0122114 |
| 123 | 123 | C6:0 | 17.12 | −0.0102934 |
| | | C8:0 | 17.54 | −0.00870533 |
| 127 | 127 | C10:0 | 16.28 | −0.0271098 |
| | | C12:0 | 12.7 | −0.029506 |
| | | C14:0 | 16.52 | −0.0426528 |
| | | C6:0 | 18.04 | −0.0118919 |
| | | C8:0 | 24.7 | −0.0106605 |
| | | DNS | 18.52 | −0.0106605 |
| 128 | 128 | C10:0 | 13.12 | −0.0232924 |
| | | C14:0 | 12.12 | −0.0358518 |
| | | C6:0 | 19.22 | −0.00997547 |
| | | C8:0 | 22.36 | −0.00893882 |
| | | DNS | 16.17 | −0.00893882 |
| 129 | 129 | C6:0 | 14.24 | −0.0142968 |
| | | C8:0 | 14.52 | −0.0118772 |
| 130 | 130 | C6:0 | 14.24 | −0.0142968 |
| | | C8:0 | 14.52 | −0.0118772 |
| 131 | 131 | C6:0 | 14.24 | −0.0142968 |
| | | C8:0 | 14.52 | −0.0118772 |
| 132 | 132 | C10:0 | 24.76 | −0.0383403 |
| | | C12:0 | 18.44 | −0.0413743 |
| | | C14:0 | 20.18 | −0.0554531 |
| | | C6:0 | 30.28 | −0.0173749 |
| | | C8:0 | 39.14 | −0.0155207 |
| | | DNS | 26.83 | −0.0155207 |
| 133 | 133 | C10:0 | 19.72 | −0.0322227 |
| | | C12:0 | 14.2 | −0.0348721 |
| | | C14:0 | 20.04 | −0.0477038 |
| | | C6:0 | 30 | −0.0154619 |
| | | C8:0 | 34.1 | −0.013215 |
| | | DNS | 22.74 | −0.013215 |
| 134 | 134 | C6:0 | 14.24 | −0.0142968 |
| | | C8:0 | 14.52 | −0.0118772 |
| 135 | 135 | C6:0 | 14.24 | −0.0142968 |
| | | C8:0 | 14.52 | −0.0118772 |
| 136 | 136 | C8:0 | 17.08 | −0.0101673 |
| 137 | 137 | C8:0 | 17.08 | −0.0101604 |
| 16 | 16 | C10:0 | 16.94 | −0.0367898 |
| | | C12:0 | 12.44 | −0.040228 |
| | | C6:0 | 14.28 | −0.0144535 |
| | | C8:0 | 23.64 | −0.0138831 |
| | | DNS | 17.03 | −0.0138831 |
| 139 | 139 | C10:0 | 23.8 | 0.028243 |
| | | C12:0 | 23.9 | 0.0335376 |
| | | C14:0 | 13.6 | 0.0443111 |
| | | C6:0 | 14.46 | 0.00718847 |
| | | C8:0 | 26.98 | 0.00897561 |
| | | DNS | 27.01 | 0.00897561 |
| 140 | 140 | C10:0 | 16.04 | −0.0294542 |
| | | C6:0 | 26.7 | −0.013484 |
| | | C8:0 | 31.84 | −0.0119712 |
| | | DNS | 20.2 | −0.0119712 |
| 224 | 224 | C10:0 | 39.1 | −0.0387091 |
| | | C12:0 | 29.76 | −0.0415053 |
| | | C14:0 | 14.86 | −0.0543235 |
| | | C6:0 | 40.56 | −0.016854 |
| | | C8:0 | 56.9 | −0.0152685 |
| | | DNS | 41.82 | −0.0152685 |
| 225 | 225 | C10:0 | 47.5 | −0.0385647 |
| | | C12:0 | 37.06 | −0.0412351 |
| | | C14:0 | 23.78 | −0.0570337 |
| | | C6:0 | 53.38 | −0.0171145 |
| | | C8:0 | 69.82 | −0.0152227 |
| | | DNS | 52.79 | −0.0152227 |
| 141 | 141 | C10:0 | 45.98 | −0.0389109 |
| | | C12:0 | 35.18 | −0.0415682 |
| | | C14:0 | 22.44 | −0.0579718 |
| | | C6:0 | 53.12 | −0.0176825 |
| | | C8:0 | 68.88 | −0.0154576 |
| | | DNS | 51.03 | −0.0154576 |
| 226 | 226 | c10:0 | 47.16 | −0.0374487 |
| | | C12:0 | 36.3 | −0.0401502 |
| | | C14:0 | 21.36 | −0.0555947 |
| | | C6:0 | 45.78 | −0.0160816 |
| | | C8:0 | 64.48 | −0.0145849 |
| | | DNS | 50.24 | −0.0145849 |
| 227 | 227 | C8:0 | 13.18 | −0.00634763 |
| 228 | 228 | C8:0 | 13.16 | −0.00634763 |
| 229 | 229 | C8:0 | 13.88 | −0.00634763 |
| 230 | 230 | C8:0 | 14.52 | −0.00634763 |
| 231 | 231 | C8:0 | 15.1 | −0.00634763 |
| | | DNS | 12 | −0.00634763 |
| 143 | 143 | C8:0 | 14.16 | −0.00711438 |
| 148 | 148 | C10:0 | 30.78 | −0.0394483 |
| | | C12:0 | 25.94 | −0.0437927 |
| | | C14:0 | 24.2 | −0.0645359 |
| | | C6:0 | 25.26 | −0.015477 |
| | | C8:0 | 35.36 | −0.0140383 |
| | | DNS | 33.09 | −0.0140383 |
| 149 | 149 | C10:0 | 15.2 | −0.0166395 |
| | | C12:0 | 15.44 | −0.0186586 |
| | | C14:0 | 15.08 | −0.0314341 |
| | | C8:0 | 12.72 | −0.0054648 |
| | | DNS | 15.74 | −0.0054648 |
| 150 | 150 | C10:0 | 15.96 | −0.0166395 |
| | | C12:0 | 16.28 | −0.0186586 |
| | | C14:0 | 16.06 | −0.0314341 |
| | | C8:0 | 13.28 | −0.0054648 |
| | | DNS | 16.62 | −0.0054648 |
| 151 | 151 | C10:0 | 14.54 | −0.0240498 |
| | | C12:0 | 12.34 | −0.0260339 |
| | | C14:0 | 13.2 | −0.0413449 |
| | | C8:0 | 15.04 | −0.00897534 |
| | | DNS | 14.97 | −0.00897534 |
| 232 | 232 | DNS | 12.41 | −0.00721868 |
| 152 | 152 | C10:0 | 13.16 | −0.0240665 |
| | | C8:0 | 13.82 | −0.00899286 |
| | | DNS | 13.37 | −0.00899286 |
| 153 | 153 | C10:0 | 24.96 | −0.0270795 |
| | | C12:0 | 20.44 | −0.0283391 |
| | | C14:0 | 21.38 | −0.0458741 |
| | | C6:0 | 21.36 | −0.0129423 |
| | | C8:0 | 31.62 | −0.0108675 |
| | | DNS | 27.2 | −0.0108675 |

### Example 3. Genome-Wide Association Analyses Using High Density Marker Data

FTIR Spectroscopy and Variance Component Estimation

FTIR predictions and estimations of genetic variance components were performed as described in Example 1, but on a larger animal material, fewer traits and a more stringent R2CV. The calibration model was applied to 3,813,049 infrared spectra from the periods February to November 2007 and July 2008 to June 2014. A total of 28 traits were calibrated for (C4:0, C6:0, C8:0, C10:0, C12:0, C14:0, C16:0, C18:0, C20:0, C15:0, C17:0, C14:1 cis-9, C16:1 cis-9, C18:1 cis-9, C18:1 cis-11, C18:1 trans-9, C18:1 trans-10, C18:1 trans-11, C18:2 cis-9,cis-12, C18:3 cis-9,cis-12,cis-15, ARA, CLA, DHA, EPA, SAT, MUFA, PUFA, and TOTAL). The acids were considered predictable if their cross-validated correlation coefficient (R2CV) was above 0.7. Estimation of variance components was performed on 2,209,486 profiles from 426,505 cattle with a pedigree of 596,581 animals. Results are shown in Table 7.

TABLE 7
Mean concentration from the GC-FID reference analyses, cross-
validated correlation coefficients (R²CV) and heritabilities (h²) for
the traits with R²CV > 0.7. C4:0 is butyric acid, C6:0
is hexanoic acid, C8:0 is octanoic acid, C10:0 is decanoic
acid, C12:0 is dodecanoic acid, C14:0 is tetradecanoic acid,
C16:0 is hexadecenoic acid, and C18:1 is oleic acid.

| Trait | Cons | R²CV | h² |
|---|---|---|---|
| C4:0 | 4.15 | 0.72 | 0.3742 |
| C6:0 | 2.48 | 0.87 | 0.2628 |
| C8:0 | 1.48 | 0.9 | 0.2073 |
| C10:0 | 3.2 | 0.9 | 0.1915 |
| C12:0 | 3.55 | 0.9 | 0.1954 |
| C14:0 | 11.22 | 0.85 | 0.1399 |
| C16:0 | 25.25 | 0.75 | 0.1606 |
| C18:1 | 21.4 | 0.94 | 0.1434 |

Genotyping

A high density SNP dataset was constructed by combining genotypes from the Affymetrix 25K SNP chip with genotypes from the Illumina BovineSNP50 BeadChip (54K) and the Illumina BovineHD Genotyping BeadChip (777K; Illumina, http://www.illumina.com). 1575 NR bulls were genotyped for the 54K chip. 536 of these bulls were also among the 2552 genotyped for the 25K chip. Next, 384 of the 1575 bulls were genotyped for the 777K chip. The three data sets were filtered to remove SNP with minor allele frequency <0.05 and positioned according to the UMD 3.1 assembly (Zimin et al., 2009). The 25K dataset was imputed to 54K before the combined 54K dataset was imputed to 777K. All imputations and phasing were performed by BEAGLE v3.3.1 (Browning and Browning, 2009). Phase information of the imputed haplotypes was utilized to identify double recombinants and correct (if possible) or remove these. The resulting dataset consisted of 1883 bulls with genotypes for 609,361 SNPs.

Statistical Analyses

A mixed linear model based single model association analysis was performed with the −mlma-loco option of the GCTA software (Yang et al., 2011). The model fitted to the performance information for each trait and each SNP was:

y=a+bx+g⁻+e

where y is the phenotype, a is the mean term, b is the additive effect (fixed effect) of the candidate SNP to be tested for association, x is the SNP genotype indicator variable coded as 0, 1 or 2, g is the polygenic effect (random effect) i.e. the accumulated effect of all SNPs except those on the chromosome where the candidate SNP is located, and e is the residual. For the ease of computation, the genetic variance, var(g), is estimated based on the null model i.e. y=a+g+e and then fixed while testing for the association between each SNP and the trait. The var(g−) will be re-estimated each time when a chromosome is excluded from calculating the GRM. A marker was considered significant if the −log(10) of its p-value was 5 or higher.

Results

Significant results were detected for all tested traits and most chromosomes. The most interesting results were detected on BTAS, 11, 13, 17, 19 and 27. P #, SEQIDNO, traits, p-values and allele substitution effects for the most relevant associations are presented in Table 8.

TABLE 8
Most significant associations detected by the genome-wide association
analyses. Trait refers to one or more fatty acids that are significantly
associated to the SNP. C4:0 is butyric acid, C6:0 is hexanoic acid,
C8:0 is octanoic acid, C10:0 is decanoic acid, C12:0 is dodecanoic acid,
C14:0 is tetradecanoic acid, C16:0 is hexadecanoic acid, C18:1 is oleic
acid. Effect is the effect of the SNP on the trait, i.e., the difference
in concentration of the fatty acid in question between the two alleles
of the SNP, measured as % by weight of total fat.

| P# | SEQIDNO | Trait | p-value | Effect |
|---|---|---|---|---|
| 233 | 233 | c18:1 | 2.25e−07 | 0.124561 |
| 234 | 234 | c18:1 | 8.76e−07 | 0.119818 |
| 235 | 235 | c18:1 | 7.41e−07 | 0.120606 |
| 236 | 236 | c18:1 | 7.41e−07 | 0.120606 |
| 237 | 237 | c18:1 | 7.41e−07 | 0.120606 |
| 238 | 238 | c18:1 | 4.97e−07 | 0.128575 |
| 239 | 239 | c18:1 | 6.21e−07 | 0.120676 |
| 240 | 240 | c18:1 | 7.23e−07 | 0.119724 |
| 241 | 241 | c16:0 | 5.05e−11 | 0.160781 |
| 242 | 242 | c18:1 | 7.54e−06 | 0.104799 |
| | | c16:0 | 4.47e−14 | 0.173165 |
| | | c4:0 | 2.4e−08 | 0.0202406 |
| 243 | 243 | c4:0 | 5.26e−10 | 0.0236089 |
| | | c16:0 | 8.65e−16 | 0.193433 |
| | | c18:1 | 5.54e−07 | 0.122794 |
| 244 | 244 | c4:0 | 5.53e−10 | 0.0236175 |
| | | c16:0 | 2.22e−15 | 0.190968 |
| | | c18:1 | 1.05e−06 | 0.119952 |
| 245 | 245 | c18:1 | 3.81e−07 | 0.124574 |
| | | c16:0 | 1.08e−15 | 0.192819 |
| | | c4:0 | 7.03e−10 | 0.0234385 |
| 246 | 246 | c4:0 | 3.17e−10 | 0.0237665 |
| | | c16:0 | 4.63e−16 | 0.19409 |
| | | c18:1 | 4.74e−07 | 0.122789 |
| 247 | 247 | c16:0 | 7.65e−12 | 0.154336 |
| 248 | 248 | c16:0 | 2.13e−11 | 0.150894 |
| 64 | 64 | c8:0 | 1.34e−11 | 0.0229585 |
| | | c10:0 | 8.21e−09 | 0.0575634 |
| 69 | 69 | c14:0 | 2.66e−07 | 0.0759402 |
| | | c12:0 | 6.19e−08 | 0.0563511 |
| | | c10:0 | 2.93e−09 | 0.05246 |
| | | c8:0 | 5.38e−12 | 0.0207033 |
| | | c6:0 | 5.09e−09 | 0.0218123 |
| 70 | 70 | c6:0 | 5.09e−09 | 0.0218123 |
| | | c8:0 | 5.38e−12 | 0.0207033 |
| | | c10:0 | 2.93e−09 | 0.05246 |
| | | c12:0 | 6.19e−08 | 0.0563511 |
| | | c14:0 | 2.66e−07 | 0.0759402 |
| 83 | 83 | c14:0 | 1.86e−07 | 0.0450041 |
| 85 | 85 | c14:0 | 1.86e−07 | 0.0450041 |
| 117 | 117 | c14:0 | 4.00E−10 | 0.0995064 |
| | | c12:0 | 1.67e−11 | 0.0755669 |
| | | c10:0 | 4.16e−13 | 0.0690894 |
| | | c8:0 | 2.87e−16 | 0.0264777 |
| | | c6:0 | 1.75e−11 | 0.0270638 |
| 118 | 118 | c12:0 | 7.46e−08 | 0.0331528 |
| 140 | 140 | c6:0 | 2.1e−09 | 0.0137123 |
| 141 | 141 | c6:0 | 3.14e−14 | 0.016716 |
| | | c8:0 | 7.33e−17 | 0.0147749 |
| | | c10:0 | 5.52e−13 | 0.0375993 |
| | | c12:0 | 3.74e−11 | 0.0406151 |
| | | c14:0 | 4.13e−10 | 0.0543995 |
| | | c18:1 | 3.53e−06 | 0.11322 |
| 148 | 148 | c18:1 | 1.05e−07 | 0.138411 |
| | | c14:0 | 1.66e−09 | 0.0559529 |
| | | c12:0 | 1.34e−09 | 0.0396809 |
| | | c10:0 | 9.85e−11 | 0.0359642 |
| | | c8:0 | 9.68e−13 | 0.0134826 |
| | | c6:0 | 3.03e−11 | 0.0156058 |
| 153 | 153 | c18:1 | 9.75e−06 | 0.10971 |
| | | c14:0 | 7.08e−08 | 0.0476701 |
| | | c10:0 | 5.77e−09 | 0.0308499 |
| | | c8:0 | 2.83e−11 | 0.0119857 |
| 156 | 156 | c8:0 | 2.03e−11 | 0.0217386 |
| 166 | 166 | c10:0 | 6.54e−09 | 0.0554598 |
| | | c8:0 | 1.4e−11 | 0.0219458 |
| 249 | 249 | c6:0 | 8.49e−09 | 0.0169082 |
| 250 | 250 | c6:0 | 8.49e−09 | 0.0169082 |
| 251 | 251 | c4:0 | 6.87e−10 | 0.0233284 |
| 252 | 252 | c4:0 | 1.52e−10 | 0.0324591 |
| 253 | 253 | c6:0 | 1.05e−09 | 0.0186681 |
| 254 | 254 | c8:0 | 2.57e−06 | 0.00798137 |
| | | c6:0 | 7.91e−10 | 0.0130226 |
| 255 | 255 | c6:0 | 1.06e−09 | 0.0130483 |
| 256 | 256 | c6:0 | 4.66e−09 | 0.0135847 |
| 257 | 257 | c6:0 | 4.66e−09 | 0.0135847 |
| 258 | 258 | c6:0 | 1.32e−10 | 0.0133183 |
| | | c8:0 | 5.04e−07 | 0.00834534 |
| 259 | 259 | c6:0 | 8.22e−11 | 0.0140297 |
| | | c8:0 | 9.72e−07 | 0.00847286 |
| 260 | 260 | c6:0 | 8.44e−09 | 0.0166838 |
| 261 | 261 | c4:0 | 3.83e−10 | 0.0305028 |
| 262 | 262 | c4:0 | 2.11e−10 | 0.0309214 |
| 263 | 263 | c4:0 | 2.02e−10 | 0.0310054 |
| 264 | 264 | c4:0 | 3.03e−10 | 0.0306546 |
| 265 | 265 | c4:0 | 2.11e−10 | 0.0309214 |
| 266 | 266 | c4:0 | 3.83e−10 | 0.0305028 |
| 267 | 267 | c6:0 | 7.16e−09 | 0.01595 |
| | | c4:0 | 1.11e−10 | 0.0306284 |
| 268 | 268 | c8:0 | 2.07e−06 | 0.00800641 |
| 269 | 269 | c4:0 | 1.42e−10 | 0.0425918 |
| 270 | 270 | c8:0 | 3.56e−07 | 0.00937742 |
| 271 | 271 | c18:1 | 3.21e−08 | 0.132082 |
| 272 | 272 | c18:1 | 3.21e−08 | 0.132082 |
| 273 | 273 | c18:1 | 3.21e−08 | 0.132082 |
| 274 | 274 | c18:1 | 1.19e−07 | 0.16861 |
| 275 | 275 | c14:0 | 1.39e−08 | 0.0605594 |
| | | c18:1 | 5.84e−09 | 0.174364 |
| 276 | 276 | c12:0 | 4.09e−07 | 0.0380963 |
| | | c14:0 | 2.42e−09 | 0.0638052 |
| | | c18:1 | 1.48e−09 | 0.181465 |
| 277 | 277 | c18:1 | 7.63e−07 | 0.155925 |
| 278 | 278 | c18:1 | 4.36e−07 | 0.160184 |
| 279 | 279 | c14:0 | 8.43e−08 | 0.0757869 |
| 280 | 280 | c14:0 | 6.84e−08 | 0.0763375 |
| | | c18:1 | 5.41e−07 | 0.198993 |
| 281 | 281 | c18:1 | 5.8e−07 | 0.198818 |
| | | c14:0 | 5.73e−08 | 0.0769278 |
| 282 | 282 | c18:1 | 9.82e−07 | 0.193985 |
| 283 | 283 | c18:1 | 9.82e−07 | 0.193985 |
| 284 | 284 | c14:0 | 5.64e−08 | 0.077563 |
| 285 | 285 | c8:0 | 6.61e−07 | 0.0134962 |
| | | c10:0 | 1.46e−08 | 0.0453257 |
| | | c12:0 | 1.46e−08 | 0.053392 |
| | | c14:0 | 1.66e−10 | 0.0855873 |
| | | c18:1 | 1.58e−08 | 0.212481 |
| 286 | 286 | c8:0 | 8.5e−07 | 0.0166128 |
| | | c10:0 | 7.58e−08 | 0.0534673 |
| | | cl2:0 | 1.21e−07 | 0.0619985 |
| | | c14:0 | 2.32e−08 | 0.0930236 |
| 287 | 287 | c8:0 | 5.75e−07 | 0.0168623 |
| | | c10:0 | 4.09e−08 | 0.0545418 |
| | | c12:0 | 6.4e−08 | 0.063328 |
| | | c14:0 | 1.19e−08 | 0.0948983 |
| 288 | 288 | c8:0 | 5.93e−08 | 0.0205638 |
| | | c10:0 | 1.59e−09 | 0.0674685 |
| | | c12:0 | 2.46e−09 | 0.0785509 |
| | | c14:0 | 2.48e−10 | 0.118462 |
| 289 | 289 | c14:0 | 9.81e−09 | 0.0494494 |
| | | c18:1 | 1.16e−06 | 0.117704 |
| 290 | 290 | c18:1 | 1.16e−06 | 0.117704 |
| | | c14:0 | 9.81e−09 | 0.0494494 |
| 291 | 291 | c14:0 | 9.81e−09 | 0.0494494 |
| | | c18:1 | 1.16e−06 | 0.117704 |
| 292 | 292 | c18:1 | 1.16e−06 | 0.117704 |
| | | c14:0 | 9.81e−09 | 0.0494494 |
| 293 | 293 | c14:0 | 9.81e−09 | 0.0494494 |
| | | c18:1 | 1.16e−06 | 0.117704 |
| 294 | 294 | c14:0 | 9.81e−09 | 0.0494494 |
| | | c18:1 | 1.16e−06 | 0.117704 |
| 295 | 295 | c18:1 | 1.16e−06 | 0.117704 |
| | | c14:0 | 9.81e−09 | 0.0494494 |
| 296 | 296 | c18:1 | 1.16e−06 | 0.117704 |
| | | c14:0 | 9.81e−09 | 0.0494494 |
| 297 | 297 | c10:0 | 2.29e−07 | 0.0267072 |
| | | c12:0 | 6.69e−08 | 0.0328482 |
| | | c14:0 | 3.61e−09 | 0.0509863 |
| | | c18:1 | 6.4e−07 | 0.120731 |
| 298 | 298 | c18:1 | 6.4e−07 | 0.120731 |
| | | c14:0 | 3.61e−09 | 0.0509863 |
| | | c12:0 | 6.69e−08 | 0.0328482 |
| | | c10:0 | 2.29e−07 | 0.0267072 |
| 299 | 299 | c18:1 | 6.4e−07 | 0.120731 |
| | | c14:0 | 3.61e−09 | 0.0509863 |
| | | c12:0 | 6.69e−08 | 0.0328482 |
| | | c10:0 | 2.29e−07 | 0.0267072 |
| 300 | 300 | c18:1 | 6.4e−07 | 0.120731 |
| | | c14:0 | 3.61e−09 | 0.0509863 |
| | | c12:0 | 6.69e−08 | 0.0328482 |
| | | c10:0 | 2.29e−07 | 0.0267072 |
| 301 | 301 | c18:1 | 5.82e−07 | 0.121087 |
| | | c14:0 | 3.89e−09 | 0.0508402 |
| | | c12:0 | 7.81e−08 | 0.0326544 |
| | | c10:0 | 2.73e−07 | 0.0265195 |
| 302 | 302 | c8:0 | 4.19e−06 | 0.0183894 |
| | | c10:0 | 8.86e−10 | 0.0722146 |
| | | c12:0 | 3.11e−10 | 0.0874091 |
| | | c14:0 | 3.29e−11 | 0.130797 |
| | | c18:1 | 6.26e−07 | 0.275795 |
| 303 | 303 | c16:0 | 3.79e−11 | 0.204002 |
| 304 | 304 | c16:0 | 6.38e−08 | 0.129454 |
| 305 | 305 | c16:0 | 5.04e−08 | 0.135218 |
| 306 | 306 | c16:0 | 5.91e−08 | 0.134387 |
| 307 | 307 | c16:0 | 4.74e−08 | 0.135212 |
| 308 | 308 | c16:0 | 3.74e−08 | 0.136219 |
| 309 | 309 | c16:0 | 7.71e−08 | 0.13307 |
| 310 | 310 | c16:0 | 3.74e−08 | 0.136219 |

### Example 4. Association Analyses Using Sequence-Level Markers in Selected Genomic Regions

The final step was to re-analyze the most significant regions detected in Example 3 using sequence-level variants on BTA11, 13, 17 and 19. Trait data was found as described in Example 3.

Whole-Genome Sequencing and Imputation

168 animals of the Norwegian Red cattle breed where sequenced. Sequencing was performed by the Norwegian Sequencing Centre, Oslo, Norway using a HiSeq 2500 platform according to the manufacturer's protocols. Samples were prepared for paired-end sequencing (2×125 bp) using TruSeq DNA PCR-free library preparation kits and sequenced with the manufacturers V4 kit (Illumina, San Diego, Calif., USA) to generate an average of 9×coverage. Sequence data from 21 Norwegian Red bulls used for artificial insemination were also available from another project (Olsen et al., unpublished). All reads were aligned against the bovine reference genome UMD 3.1, using BWA-mein version 0.7.10 (Li, 2013). Variant calling was done with FreeBayes version 1.0.2 (Garrison & Marth, 2012). Genotypes of the called variants were refined and phased using Beagle version 4.1 (Browning & Browning, 2009). The resulting phased dataset was then used as a reference panel for imputing 1816 animals to full sequence, also using Beagle 4.1.

Statistical Analyses

Association analyses were performed with the ASREML package version 2.0 (Gilmour et al., 2006). The model that was fitted to the information on performance for each trait—marker combination was:

DYD=1μXb+Za+e,

where DYD is the vector of bull performances weighed by the number of daughters, 1 is a vector of ones, μ is the overall mean, X is a vector of SNP genotypes coded as 0, 1, or 2 depending on the number of copies of the first allele, b is the fixed effect of the marker, Z is an incidence matrix relating phenotypes to the corresponding random polygenic effects, a is a vector of random polygenic effects, and e is a vector of residual effects. Genetic and residual variances were estimated from the data. a was assumed to follow a normal distribution ˜N(0,Aσ_{A}²) where A is the relationship matrix derived from the pedigree, and σ_{A}² is the additive genetic variance. e was assumed to follow a normal distribution ˜N(0, Ioₑ²) where σₑ² is the residual variance. Association analysis was performed for each individual marker, and then the p-value for the marker effect was calculated with the R function pf( ).

Results

For all tested chromosomes (i.e., BTA11, 13, 17 and 19), highly significant associations were detected close to or within known candidate genes. P #, SEQIDNO, traits, p-values and allele substitution effects for the most relevant associations are presented in Table 9.

TABLE 9
Significant associations detected by the genome-wide association
analyses. Trait refers to one or more fatty acids that are significantly
associated to the polymorphism. Effect is the effect of the polymorphism
on the trait, i.e., the difference in concentration of the fatty
acid in question between the two alleles of the polymorphism,
measured as % by weight of total fat.

| P# | SEQIDNO | Trait | P-value | Effect |
|---|---|---|---|---|
| 117 | 117 | C8:0 | 2.3014830551197799E−16 | 0.02815 |
| | | C10:0 | 5.8976455450284501E−13 | 0.0732 |
| | | C12:0 | 2.5389592799163501E−11 | 0.08002 |
| | | C14:0 | 3.7247485266275898E−11 | 0.1121 |
| 141 | 141 | C6:0 | 1.3070524624908399E−15 | 0.01795 |
| | | C8:0 | 8.0096072416566797E−18 | 0.01557 |
| | | C10:0 | 1.1989284537494899E−13 | 0.03978 |
| | | C12:0 | 1.8801231438726598E−11 | 0.04251 |
| | | C14:0 | 9.7948456090688495E−12 | 0.06081 |
| 148 | 148 | C12:0 | 3.03855288409611E−11 | 0.04825 |
| | | C14:0 | 5.2870202455405602E−12 | 0.0705 |
| | | C18:1cis-9 | 1.66685761535244E−8 | 0.1643 |
| 224 | 224 | C6:0 | 1.67177926214398E−15 | 0.01803 |
| | | C8:0 | 7.4456438235387396E−18 | 0.01571 |
| | | C10:0 | 1.4255384497315401E−13 | 0.03999 |
| | | C12:0 | 2.2163898231281201E−11 | 0.04272 |
| | | C14:0 | 4.7503119381623603E−11 | 0.05927 |
| 225 | 225 | C6:0 | 6.8224346074786399E−16 | 0.01826 |
| | | C8:0 | 7.2315971463352595E−18 | 0.01569 |
| | | C10:0 | 1.5887627833528101E−13 | 0.03986 |
| | | C12:0 | 2.8573276932427101E−11 | 0.04243 |
| | | C14:0 | 1.2010385440067199E−11 | 0.06098 |
| 226 | 226 | C6:0 | 5.1094854026620902E−15 | 0.0174 |
| | | C8:0 | 1.86730902209013E−17 | 0.01524 |
| | | C10:0 | 1.01891692801114E−13 | 0.0395 |
| | | C12:0 | 1.4659211231729599E−11 | 0.04232 |
| | | C14:0 | 3.6985796833596601E−12 | 0.06142 |
| 242 | 242 | C4:0 | 1.2928384351839337e−7 | 0.02075 |
| | | C16:0 | 2.2630269905553917e−12 | 0.1758 |
| | | C18:1cis-9 | 4.756542897324087e−6 | 0.1174 |
| 243 | 243 | C4:0 | 1.8152184244684335e−8 | 0.02268 |
| | | C16:0 | 5.095336086824539e−13 | 0.1856 |
| | | C14:1cis-9 | 1.7203708485289344e−32 | 0.01703 |
| | | C18:1cis-9 | 7.531875593020596e−6 | 0.118 |
| 244 | 244 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 245 | 245 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 246 | 246 | C4:0 | 3.011420785499756e−8 | 0.02242 |
| | | C16:0 | 3.901378661041524e−13 | 0.1872 |
| | | C14:1cis-9 | 8.701149888615851e−32 | 0.01691 |
| | | C18:1cis-9 | 2.5899127171794412e−6 | 0.1243 |
| 247 | 247 | C4:0 | 4.6019784418698985e−7 | 0.01905 |
| 290 | 290 | C12:0 | 5.0351573929741803E−6 | 0.02967 |
| 291 | 291 | C12:0 | 5.5255980595812297E−6 | 0.02931 |
| | | C14:0 | 8.8207972109658898E−7 | 0.04464 |
| 292 | 292 | C12:0 | 7.9329768149947606E−6 | 0.02904 |
| 293 | 293 | C12:0 | 7.9329768149947606E−6 | 0.02904 |
| 294 | 294 | C12:0 | 7.9329768149947606E−6 | 0.02904 |
| 295 | 295 | C12:0 | 7.9329768149947606E−6 | 0.02904 |
| 296 | 296 | C12:0 | 5.7884041878071203E−6 | 0.02925 |
| | | C14:0 | 9.1428382189332197E−7 | 0.04458 |
| 302 | 302 | C10:0 | 1.4385618706753499E−8 | 0.06922 |
| | | C12:0 | 1.09508753457544E−8 | 0.08223 |
| | | C14:0 | 1.07501268719868E−9 | 0.1236 |
| 311 | 311 | C4:0 | 1.5137820662783228e−7 | 0.02128 |
| | | C16:0 | 2.437824562717747e−12 | 0.1811 |
| | | C18:1cis-9 | 2.7266827435303454e−6 | 0.1242 |
| 312 | 312 | C4:0 | 1.5137820662783228e−7 | 0.02128 |
| | | C16:0 | 2.437824562717747e−12 | 0.1811 |
| | | C18:1cis-9 | 2.7266827435303454e−6 | 0.1242 |
| 313 | 313 | C4:0 | 2.1294923450168264e−7 | 0.02045 |
| | | C18:1cis-9 | 7.650150055787524e−6 | 0.1152 |
| 314 | 314 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 315 | 315 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 316 | 316 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 317 | 317 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 318 | 318 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 319 | 319 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 320 | 320 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 321 | 321 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 322 | 322 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 323 | 323 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 324 | 324 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 325 | 325 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 326 | 326 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 327 | 327 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 328 | 328 | C4:0 | 7.278974349272435e−8 | 0.02197 |
| | | C16:0 | 1.734970266060783e−13 | 0.1916 |
| | | C14:1cis-9 | 2.4769509682289905e−31 | 0.01693 |
| | | C18:1cis-9 | 1.2109416972909078e−6 | 0.1294 |
| 329 | 329 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 330 | 330 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 331 | 331 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 332 | 332 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 333 | 333 | C16:0 | 8.114314687461337e−13 | 0.1872 |
| | | C18:1cis-9 | 9.714062226470646e−7 | 0.1312 |
| 334 | 334 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 335 | 335 | C4:0 | 1.5767373661220744e−7 | 0.0221 |
| | | C16:0 | 6.3553468887018205e−12 | 0.1847 |
| | | C14:1cis-9 | 1.1235150399561972e−30 | 0.01729 |
| 336 | 336 | C4:0 | 1.5767373661220744e−7 | 0.0221 |
| | | C16:0 | 6.3553468887018205e−12 | 0.1847 |
| | | C14:1cis-9 | 1.1235150399561972e−30 | 0.01729 |
| 337 | 337 | C4:0 | 1.5767373661220744e−7 | 0.0221 |
| | | C16:0 | 6.3553468887018205e−12 | 0.1847 |
| | | C14:1cis-9 | 1.1235150399561972e−30 | 0.01729 |
| 338 | 338 | C4:0 | 1.353447649674352e−7 | 0.02223 |
| | | C16:0 | 5.811599778385854e−12 | 0.1851 |
| | | C14:1cis-9 | 1.4735867085928354e−30 | 0.01726 |
| 339 | 339 | C4:0 | 4.494458973358419e−8 | 0.02271 |
| | | C16:0 | 8.399593306089093e−13 | 0.1894 |
| | | C14:1cis-9 | 6.771136889228095e−32 | 0.01738 |
| | | C18:1cis-9 | 8.750764458845112e−6 | 0.1206 |
| 340 | 340 | C4:0 | 1.7636328224756067e−7 | 0.02149 |
| | | C16:0 | 7.645371862105105e−13 | 0.188 |
| | | C14:1cis-9 | 5.0024549398183224e−30 | 0.0167 |
| | | C18:1cis-9 | 2.7548250183340124e−6 | 0.126 |
| 341 | 341 | C4:0 | 7.442792433548095e−7 | 0.02062 |
| | | C16:0 | 3.2852640568732234e−12 | 0.185 |
| 342 | 342 | C4:0 | 7.71434569875298e−7 | 0.02059 |
| | | C16:0 | 3.3179747247742437e−12 | 0.1849 |
| 343 | 343 | C4:0 | 9.721829816678287e−7 | 0.0198 |
| | | C16:0 | 6.103136316054401e−13 | 0.1854 |
| | | C18:1cis-9 | 9.888231648289864e−7 | 0.1291 |
| 344 | 344 | C16:0 | 4.192896736223586e−13 | 0.1874 |
| | | C18:1cis-9 | 1.241649214752762e−6 | 0.1284 |
| 345 | 345 | C18:1cis-9 | 9.485667292863972e−6 | 0.1367 |
| 346 | 346 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 347 | 347 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 348 | 348 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 349 | 349 | C4:0 | 7.205410314354753e−8 | 0.02199 |
| | | C16:0 | 2.403578558716973e−13 | 0.1906 |
| | | C14:1cis-9 | 2.7451035248502325e−31 | 0.01693 |
| | | C18:1cis-9 | 1.255213977040492e−6 | 0.1293 |
| 350 | 350 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 351 | 351 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 352 | 352 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 353 | 353 | C4:0 | 7.143926265738729e−7 | 0.02011 |
| | | C16:0 | 6.493404779341801e−13 | 0.1858 |
| | | C18:1cis-9 | 3.4731332592943718e−6 | 0.1229 |
| 354 | 354 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 55 | 355 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 356 | 356 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 357 | 357 | C4:0 | 6.443792098934717e−8 | 0.02207 |
| | | C16:0 | 2.5253095891876994e−13 | 0.1904 |
| | | C14:1cis-9 | 2.7447244560589406e−31 | 0.01693 |
| | | C18:1cis-9 | 1.2360469599051865e−6 | 0.1293 |
| 358 | 358 | C4:0 | 3.3665599590093695e−8 | 0.0224 |
| | | C16:0 | 2.231812198071488e−13 | 0.1897 |
| | | C14:1cis-9 | 6.113418920441182e−32 | 0.017 |
| | | C18:1cis-9 | 2.195605222243276e−7 | 0.1373 |
| 359 | 359 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 360 | 360 | C4:0 | 7.132640516922274e−8 | 0.02184 |
| | | C16:0 | 1.3094322833208976e−13 | 0.1913 |
| | | C14:1cis-9 | 1.8730971384864796e−31 | 0.01684 |
| | | C18:1cis-9 | 4.0199932688293885e−8 | 0.1452 |
| 361 | 361 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 362 | 362 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 363 | 363 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 364 | 364 | C4:0 | 8.812704304509378e−7 | 0.02024 |
| | | C16:0 | 6.066803065128827e−14 | 0.1967 |
| | | C18:1cis-9 | 7.144032066720917e−7 | 0.1332 |
| 365 | 365 | C4:0 | 7.995573787063973e−7 | 0.02032 |
| | | C16:0 | 7.839591476556987e−14 | 0.1959 |
| | | C18:1cis-9 | 8.036775888619343e−7 | 0.1326 |
| 366 | 366 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 367 | 367 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 368 | 368 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 369 | 369 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 370 | 370 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 371 | 371 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 372 | 372 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 373 | 373 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 374 | 374 | C4:0 | 6.346390796548164e−8 | 0.02209 |
| | | C16:0 | 2.500479056866569e−13 | 0.1905 |
| | | C14:1cis-9 | 2.7968934535856455e−31 | 0.01693 |
| | | C18:1cis-9 | 1.2297178880669441e−6 | 0.1294 |
| 375 | 375 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 376 | 376 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 377 | 377 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 378 | 378 | C4:0 | 6.346390796548164e−8 | 0.02209 |
| | | C16:0 | 2.500479056866569e−13 | 0.1905 |
| | | C14:1cis-9 | 2.7968934535856455e−31 | 0.01693 |
| | | C18:1cis-9 | 1.2297178880669441e−6 | 0.1294 |
| 379 | 379 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 380 | 380 | C4:0 | 6.346390796548164e−8 | 0.02209 |
| | | C16:0 | 2.500479056866569e−13 | 0.1905 |
| | | C14:1cis-9 | 2.7968934535856455e−31 | 0.01693 |
| | | C18:1cis-9 | 1.2297178880669441e−6 | 0.1294 |
| 381 | 381 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 382 | 382 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 383 | 383 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 384 | 384 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 385 | 385 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 386 | 386 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 387 | 387 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 388 | 388 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 389 | 389 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 390 | 390 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 391 | 391 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 392 | 392 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 393 | 393 | C4:0 | 2.1559811336931593e−8 | 0.02249 |
| | | C16:0 | 9.942389518170167e−13 | 0.1827 |
| | | C18:1cis-9 | 1.9926107976021747e−6 | 0.1248 |
| 394 | 394 | C4:0 | 3.577620016917801e−8 | 0.02231 |
| | | C16:0 | 1.1865936126672857e−13 | 0.1914 |
| | | C14:1cis-9 | 1.3072036972397023e−30 | 0.0166 |
| | | C18:1cis-9 | 7.254826833945297e−7 | 0.131 |
| 395 | 395 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 396 | 396 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 397 | 397 | C4:0 | 1.4313954046104633e−7 | 0.02177 |
| | | C16:0 | 1.0332931244476925e−13 | 0.196 |
| | | C18:1cis-9 | 9.516673708273554e−7 | 0.1324 |
| 398 | 398 | C4:0 | 1.1384816126435856e−7 | 0.02191 |
| | | C16:0 | 5.6064937335358955e−14 | 0.1978 |
| | | C14:1cis-9 | 1.5533049883745766e−30 | 0.01691 |
| | | C18:1cis-9 | 5.071147162114983e−7 | 0.1355 |
| 399 | 399 | C4:0 | 5.365050090229759e−7 | 0.02021 |
| | | C16:0 | 1.391247960614076e−13 | 0.1899 |
| | | C14:1cis-9 | 9.675984237123455e−32 | 0.01684 |
| | | C18:1cis-9 | 1.8643345505033625e−6 | 0.1254 |
| 400 | 400 | C4:0 | 8.139698937794954e−8 | 0.02191 |
| | | C16:0 | 2.439412688304182e−13 | 0.1907 |
| | | C14:1cis-9 | 2.5847556040758126e−31 | 0.01694 |
| | | C18:1cis-9 | 1.1155508871296394e−6 | 0.13 |
| 401 | 401 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 402 | 402 | C4:0 | 1.3743120059032624e−7 | 0.02143 |
| | | C16:0 | 9.79468415499461e−14 | 0.1929 |
| | | C14:1cis-9 | 1.5105995593840388e−30 | 0.01667 |
| | | C18:1cis-9 | 2.2428579889633397e−6 | 0.1257 |
| 403 | 403 | C4:0 | 1.3743120059032624e−7 | 0.02143 |
| | | C16:0 | 9.79468415499461e−14 | 0.1929 |
| | | C14:1cis-9 | 1.5105995593840388e−30 | 0.01667 |
| | | C18:1cis-9 | 2.2428579889633397e−6 | 0.1257 |
| 404 | 404 | C4:0 | 6.443792098934717e−8 | 0.02208 |
| | | C16:0 | 2.475893088729249e−13 | 0.1905 |
| | | C14:1cis-9 | 2.8492685771524566e−31 | 0.01693 |
| | | C18:1cis-9 | 1.2424030932700843e−6 | 0.1293 |
| 405 | 405 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 406 | 406 | C4:0 | 1.8184395005057115e−7 | 0.0215 |
| | | C16:0 | 8.574332970891014e−14 | 0.1958 |
| | | C14:1cis-9 | 1.9635728823887853e−30 | 0.01685 |
| | | C18:1cis-9 | 1.9623263053383544e−6 | 0.128 |
| 407 | 407 | C4:0 | 1.7344347301554467e−8 | 0.02279 |
| | | C16:0 | 2.34482969221407e−13 | 0.1889 |
| | | C14:1cis-9 | 3.206335307398801e−31 | 0.01674 |
| | | C18:1cis-9 | 8.24504574505618e−7 | 0.1302 |
| 408 | 408 | C4:0 | 1.769883723743664e−8 | 0.02276 |
| | | C16:0 | 2.2651322023310465e−13 | 0.1889 |
| | | C14:1cis-9 | 3.297514139655115e−31 | 0.01673 |
| | | C18:1cis-9 | 8.20293094909458e−7 | 0.1302 |
| 409 | 409 | C4:0 | 9.625863506374483e−8 | 0.02201 |
| | | C16:0 | 4.1396971873752493e−13 | 0.1907 |
| | | C14:1cis-9 | 2.571995323610433e−31 | 0.01711 |
| | | C18:1cis-9 | 2.04434028213731e−6 | 0.128 |
| 410 | 410 | C4:0 | 9.432119242564688e−8 | 0.02202 |
| | | C16:0 | 3.4647234925139456e−13 | 0.1913 |
| | | C14:1cis-9 | 2.6950184884574883e−31 | 0.01711 |
| | | C18:1cis-9 | 1.7433347196914435e−6 | 0.1289 |
| 411 | 411 | C4:0 | 8.306762983283962e−8 | 0.02188 |
| | | C16:0 | 2.36832509070101e−13 | 0.1906 |
| | | C18:1cis-9 | 1.132858006469425e−6 | 0.1297 |
| 412 | 412 | C4:0 | 1.7878797515737317e−8 | 0.02281 |
| | | C16:0 | 2.7060583938165915e−13 | 0.1887 |
| | | C14:1cis-9 | 2.8130986201265264e−31 | 0.01679 |
| | | C18:1cis-9 | 1.0120206967655212e−6 | 0.1294 |
| 413 | 413 | C4:0 | 1.7878797515737317e−8 | 0.02281 |
| | | C16:0 | 2.7060583938165915e−13 | 0.1887 |
| | | C14:1cis-9 | 2.8130986201265264e−31 | 0.01679 |
| | | C18:1cis-9 | 1.0120206967655212e−6 | 0.1294 |
| 414 | 414 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 415 | 415 | C4:0 | 4.6805407049219326e−8 | 0.02223 |
| | | C16:0 | 4.098958348828484e−13 | 0.1881 |
| | | C14:1cis-9 | 3.19491320822314e−32 | 0.01712 |
| | | C18:1cis-9 | 3.1983961715065176e−6 | 0.1238 |
| 416 | 416 | C4:0 | 4.75230955988751e−8 | 0.02225 |
| | | C16:0 | 3.676483332686772e−13 | 0.1888 |
| | | C14:1cis-9 | 3.813440085778458e−32 | 0.01713 |
| | | C18:1cis-9 | 2.75484697268075e−6 | 0.1248 |
| 417 | 417 | C4:0 | 1.1634008599491163e−8 | 0.02304 |
| | | C16:0 | 1.0762761292334859e−12 | 0.1835 |
| | | C14:1cis-9 | 9.283687196248366e−31 | 0.01661 |
| | | C18:1cis-9 | 1.261783342372347e−6 | 0.1279 |
| 418 | 418 | C4:0 | 1.1634008599491163e−8 | 0.02283 |
| | | C16:0 | 5.329125986254982e−13 | 0.1842 |
| | | C14:1cis-9 | 7.1437372441913e−31 | 0.01649 |
| | | C18:1cis-9 | 4.229600405536392e−8 | 0.1432 |
| 419 | 419 | C4:0 | 3.4669246927481577e−9 | 0.02373 |
| | | C16:0 | 3.0637351462276833e−12 | 0.1789 |
| | | C14:1cis-9 | 2.650905951040827e−31 | 0.01668 |
| | | C18:1cis-9 | 1.1804042528860565e−6 | 0.1276 |
| 420 | 420 | C4:0 | 7.348018678688381e−9 | 0.02326 |
| | | C16:0 | 9.7471771406907e−13 | 0.1831 |
| | | C14:1cis-9 | 8.376623785752058e−31 | 0.01656 |
| | | C18:1cis-9 | 1.3539395131267877e−8 | 0.1491 |
| 421 | 421 | C4:0 | 6.189854495279285e−9 | 0.02355 |
| | | C16:0 | 2.4025426097180356e−12 | 0.1812 |
| | | C14:1cis-9 | 1.3753815674381856e−30 | 0.01662 |
| | | C18:1cis-9 | 4.7214763557842583e−7 | 0.1334 |
| 422 | 422 | C4:0 | 6.0662758585827845e−9 | 0.02358 |
| | | C16:0 | 3.048682564573627e−12 | 0.1805 |
| | | C14:1cis-9 | 3.0354445535356497e−30 | 0.01654 |
| | | C18:1cis-9 | 6.684979946696033e−7 | 0.1317 |
| 423 | 423 | C4:0 | 4.999606867873136e−8 | 0.02225 |
| | | C16:0 | 2.488155653647394e−13 | 0.1904 |
| | | C14:1cis-9 | 6.339440522103002e−32 | 0.0171 |
| | | C18:1cis-9 | 1.07072951258652e−6 | 0.1301 |
| 424 | 424 | C4:0 | 3.860214491652213e−8 | 0.02242 |
| | | C16:0 | 3.185474007525172e−13 | 0.1894 |
| | | C14:1cis-9 | 5.163345070037479e−32 | 0.01711 |
| | | C18:1cis-9 | 1.1212902389279808e−6 | 0.1297 |
| 425 | 425 | C4:0 | 3.3836124043964913e−8 | 0.0225 |
| | | C16:0 | 3.7314190059794324e−13 | 0.1888 |
| | | C14:1cis-9 | 4.815234483620888e−32 | 0.0171 |
| | | C18:1cis-9 | 1.16824218168249e−6 | 0.1294 |
| 426 | 426 | C4:0 | 4.6805407049219326e−8 | 0.02229 |
| | | C16:0 | 2.6012349668551466e−13 | 0.1903 |
| | | C14:1cis-9 | 6.135801782045503e−32 | 0.0171 |
| | | C18:1cis-9 | 1.07072951258652e−6 | 0.13 |
| 427 | 427 | C4:0 | 3.999663694839671e−8 | 0.02241 |
| | | C16:0 | 2.8291852447940065e−13 | 0.19 |
| | | C14:1cis-9 | 1.641110214369391e−32 | 0.01726 |
| | | C18:1cis-9 | 1.6391420527725762e−6 | 0.1278 |
| 428 | 428 | C4:0 | 4.9743070974814015e−8 | 0.02225 |
| | | C16:0 | 2.439412688304182e−13 | 0.1905 |
| | | C14:1cis-9 | 6.339440522103002e−32 | 0.0171 |
| | | C18:1cis-9 | 1.0652548554351088e−6 | 0.1301 |
| 429 | 429 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 430 | 430 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 431 | 431 | C4:0 | 6.346390796548164e−8 | 0.02209 |
| | | C16:0 | 2.653216867291837e−13 | 0.1903 |
| | | C14:1cis-9 | 2.783854972863858e−31 | 0.01693 |
| | | C18:1cis-9 | 1.2360413508923763e−6 | 0.1293 |
| 432 | 432 | C4:0 | 5.233192492552268e−8 | 0.02213 |
| | | C16:0 | 9.50244078190953e−14 | 0.1929 |
| | | C14:1cis-9 | 8.996521334901145e−33 | 0.01726 |
| | | C18:1cis-9 | 6.82250700871394e−7 | 0.1319 |
| 433 | 433 | C4:0 | 5.050594250300961e−8 | 0.02216 |
| | | C16:0 | 9.316797075465843e−14 | 0.193 |
| | | C14:1cis-9 | 9.33776713381136e−33 | 0.01726 |
| | | C18:1cis-9 | 6.787683291596898e−7 | 0.1319 |
| 434 | 434 | C4:0 | 5.233192492552268e−8 | 0.02213 |
| | | C16:0 | 9.50244078190953e−14 | 0.1929 |
| | | C14:1cis-9 | 8.996521334901145e−33 | 0.01726 |
| | | C18:1cis-9 | 6.82250700871394e−7 | 0.1319 |
| 435 | 435 | C4:0 | 5.233192492552268e−8 | 0.02213 |
| | | C16:0 | 9.50244078190953e−14 | 0.1929 |
| | | C14:1cis-9 | 8.996521334901145e−33 | 0.01726 |
| | | C18:1cis-9 | 6.82250700871394e−7 | 0.1319 |
| 436 | 436 | C4:0 | 6.443792098934717e−8 | 0.02207 |
| | | C16:0 | 2.512863600755294e−13 | 0.1905 |
| | | C14:1cis-9 | 2.7319294110163084e−31 | 0.01693 |
| | | C18:1cis-9 | 1.2360469599051865e−6 | 0.1293 |
| 437 | 437 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 438 | 438 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 439 | 439 | C4:0 | 6.476591276091663e−8 | 0.02207 |
| | | C16:0 | 2.5378173275509097e−13 | 0.1904 |
| | | C14:1cis-9 | 2.7447244560589406e−31 | 0.01693 |
| | | C18:1cis-9 | 1.2360469599051865e−6 | 0.1293 |
| 440 | 440 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 441 | 441 | C4:0 | 6.779423288772559e−8 | 0.02183 |
| | | C16:0 | 3.6227528835872935e−13 | 0.1874 |
| | | C14:1cis-9 | 6.3324542156145576e−31 | 0.01667 |
| | | C18:1cis-9 | 1.4792508710959468e−6 | 0.1272 |
| 442 | 442 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 443 | 443 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 444 | 444 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 445 | 445 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 446 | 446 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 447 | 447 | C4:0 | 3.249192944267236e−8 | 0.02236 |
| | | C16:0 | 3.297692175654934e−13 | 0.1878 |
| | | C14:1cis-9 | 1.5967584472209152e−31 | 0.01684 |
| | | C18:1cis-9 | 2.8121560494414533e−6 | 0.1238 |
| 448 | 448 | C4:0 | 3.249192944267236e−8 | 0.02236 |
| | | C16:0 | 3.297692175654934e−13 | 0.1878 |
| | | C14:1cis-9 | 1.5967584472209152e−31 | 0.01684 |
| | | C18:1cis-9 | 2.8121560494414533e−6 | 0.1238 |
| 449 | 449 | C4:0 | 1.0357668021584458e−8 | 0.02278 |
| | | C16:0 | 1.4886947039786878e−13 | 0.1875 |
| | | C14:1cis-9 | 1.8659338846662636e−31 | 0.01655 |
| | | C18:1cis-9 | 8.563916602878727e−8 | 0.1392 |
| 450 | 450 | C4:0 | 3.523609489908919e−8 | 0.02229 |
| | | C16:0 | 3.169907723250786e−13 | 0.1878 |
| | | C14:1cis-9 | 2.5347927796226416e−31 | 0.01677 |
| | | C18:1cis-9 | 2.826677856104394e−6 | 0.1237 |
| 451 | 451 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 452 | 452 | C4:0 | 2.891826347380931e−8 | 0.02245 |
| | | C16:0 | 2.7061789219454134e−13 | 0.1886 |
| | | C14:1cis-9 | 1.3124265867869915e−31 | 0.01687 |
| | | C18:1cis-9 | 2.174421685370506e−6 | 0.1252 |
| 453 | 453 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 454 | 454 | C4:0 | 3.595771825173331e−8 | 0.02231 |
| | | C16:0 | 1.309462987689054e−13 | 0.1912 |
| | | C14:1cis-9 | 3.719509922115289e−31 | 0.01675 |
| | | C18:1cis-9 | 3.771598489537187e−6 | 0.1223 |
| 455 | 455 | C4:0 | 3.595771825173331e−8 | 0.02231 |
| | | C16:0 | 1.309462987689054e−13 | 0.1912 |
| | | C14:1cis-9 | 3.719509922115289e−31 | 0.01675 |
| | | C18:1cis-9 | 3.771598489537187e−6 | 0.1223 |
| 456 | 456 | C4:0 | 2.1342350383768972e−8 | 0.02262 |
| | | C16:0 | 1.5874008557626008e−13 | 0.1901 |
| | | C14:1cis-9 | 1.9889611657374917e−31 | 0.01679 |
| | | C18:1cis-9 | 1.6731590847585173e−6 | 0.1264 |
| 457 | 457 | C4:0 | 2.123461373479123e−8 | 0.02263 |
| | | C16:0 | 1.564064291826473e−13 | 0.1901 |
| | | C14:1cis-9 | 2.0076282248558306e−31 | 0.01679 |
| | | C18:1cis-9 | 1.6645889394847738e−6 | 0.1264 |
| 458 | 458 | C4:0 | 4.1441656818356155e−8 | 0.02204 |
| | | C16:0 | 1.3452000582278674e−14 | 0.1971 |
| | | C14:1cis-9 | 4.658577156152001e−32 | 0.01687 |
| | | C18:1cis-9 | 1.0875609059743566e−7 | 0.1393 |
| 459 | 459 | C4:0 | 4.1441656818356155e−8 | 0.02204 |
| | | C16:0 | 1.3452000582278674e−14 | 0.1971 |
| | | C14:1cis-9 | 4.658577156152001e−32 | 0.01687 |
| | | C18:1cis-9 | 1.0875609059743566e−7 | 0.1393 |
| 460 | 460 | C4:0 | 2.9065112163706328e−8 | 0.02253 |
| | | C16:0 | 9.643870137669794e−14 | 0.1928 |
| | | C14:1cis-9 | 6.468699750053031e−30 | 0.01646 |
| | | C18:1cis-9 | 3.439605203642688e−7 | 0.1352 |
| 461 | 461 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 462 | 462 | C4:0 | 2.9810649930934736e−8 | 0.02243 |
| | | C16:0 | 4.525190120362981e−13 | 0.1868 |
| | | C14:1cis-9 | 1.4478237941937539e−31 | 0.01686 |
| | | C18:1cis-9 | 3.1656254410155834e−6 | 0.1233 |
| 463 | 463 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 464 | 464 | C4:0 | 3.8212749674616415e−8 | 0.02236 |
| | | C16:0 | 9.982923596453529e−14 | 0.1929 |
| | | C14:1cis-9 | 4.0653359243903524e−30 | 0.01654 |
| | | C18:1cis-9 | 3.13772277624039e−7 | 0.1358 |
| 465 | 465 | C4:0 | 2.891826347380931e−8 | 0.02246 |
| | | C16:0 | 2.588471941432331e−13 | 0.1887 |
| | | C14:1cis-9 | 1.3687458227832632e−31 | 0.01687 |
| | | C18:1cis-9 | 2.1521795116735227e−6 | 0.1253 |
| 466 | 466 | C4:0 | 6.00171654737797e−8 | 0.02189 |
| | | C16:0 | 1.4814330923969496e−13 | 0.1902 |
| | | C14:1cis-9 | 4.046571109259362e−31 | 0.0167 |
| | | C18:1cis-9 | 1.622394823542191e−6 | 0.1265 |
| 467 | 467 | C4:0 | 4.186466514671147e−8 | 0.02202 |
| | | C16:0 | 4.1807514204675e−13 | 0.1857 |
| | | C14:1cis-9 | 1.011834685681615e−31 | 0.01677 |
| | | C18:1cis-9 | 5.4676200339773355e−6 | 0.1193 |
| 468 | 468 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 469 | 469 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 470 | 470 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 471 | 471 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 472 | 472 | C4:0 | 2.4467903856632792e−8 | 0.02268 |
| | | C16:0 | 5.095552826280808e−13 | 0.1873 |
| | | C14:1cis-9 | 1.5676510406490099e−31 | 0.01693 |
| | | C18:1cis-9 | 1.7344036212755845e−6 | 0.1271 |
| 473 | 473 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 474 | 474 | C4:0 | 2.8772158824693984e−8 | 0.02246 |
| | | C16:0 | 2.692839941936475e−13 | 0.1886 |
| | | C14:1cis-9 | 1.3124265867869915e−31 | 0.01687 |
| | | C18:1cis-9 | 2.1968947070395785e−6 | 0.1252 |
| 475 | 475 | C4:0 | 2.8772158824693984e−8 | 0.02245 |
| | | C16:0 | 2.692839941936475e−13 | 0.1886 |
| | | C14:1cis-9 | 1.3124265867869915e−31 | 0.01687 |
| | | C18:1cis-9 | 2.185629190341092e−6 | 0.1252 |
| 476 | 476 | C4:0 | 7.44290206536497e−7 | 0.01877 |
| 477 | 477 | C4:0 | 2.8772158824693984e−8 | 0.02245 |
| | | C16:0 | 2.692839941936475e−13 | 0.1886 |
| | | C14:1cis-9 | 1.3124265867869915e−31 | 0.01687 |
| | | C18:1cis-9 | 2.185629190341092e−6 | 0.1252 |
| 478 | 478 | C4:0 | 1.2677140785248468e−8 | 0.02289 |
| | | C16:0 | 2.653157694797189e−13 | 0.1876 |
| | | C14:1cis-9 | 4.317494454151923e−33 | 0.01717 |
| | | C18:1cis-9 | 1.5333022239870944e−6 | 0.1264 |
| 479 | 479 | C4:0 | 1.7973821612500078e−8 | 0.02258 |
| | | C16:0 | 3.4547738903348437e−13 | 0.186 |
| | | C14:1cis-9 | 1.541015639769122e−32 | 0.01698 |
| | | C18:1cis-9 | 4.035230831530114e−6 | 0.1208 |
| 480 | 480 | C4:0 | 2.8773914333859296e−8 | 0.02214 |
| | | C16:0 | 6.152748171738368e−13 | 0.1831 |
| | | C14:1cis-9 | 3.4592948055240004e−33 | 0.01705 |
| | | C18:1cis-9 | 7.810598625171563e−6 | 0.1165 |
| 481 | 481 | C4:0 | 1.9782323131435696e−8 | 0.02265 |
| | | C16:0 | 4.306749252971704e−13 | 0.1864 |
| | | C14:1cis-9 | 1.86100369806756e−32 | 0.01705 |
| | | C18:1cis-9 | 5.008376049131687e−6 | 0.1204 |
| 482 | 482 | C4:0 | 7.144243777499045e−7 | 0.01881 |
| 483 | 483 | C14:0 | 3.97527101522368E−11 | 0.07208 |
| 484 | 484 | C14:0 | 5.54360447823387E−12 | 0.07474 |
| | | C18:1cis-9 | 9.3367189098250398E−8 | 0.1623 |
| 485 | 485 | C14:0 | 5.54360447823387E−12 | 0.07474 |
| | | C18:1cis-9 | 9.3367189098250398E−8 | 0.1623 |
| 486 | 486 | C14:0 | 5.3271734853319101E−12 | 0.07482 |
| | | C18:1cis-9 | 9.1488120708533204E−8 | 0.1625 |
| 487 | 487 | C12:0 | 4.0196419308599601E−11 | 0.05107 |
| | | C14:0 | 4.1348380093633499E−12 | 0.07585 |
| | | C18:1cis-9 | 9.3842974698324402E−8 | 0.1637 |
| 488 | 488 | C12:0 | 4.90176628620921E−12 | 0.05378 |
| | | C14:0 | 1.4189374400058499E−12 | 0.07804 |
| | | C18:1cis-9 | 6.4113364373631603E−8 | 0.1669 |
| 489 | 489 | C12:0 | 4.7341555642885902E−12 | 0.05381 |
| | | C14:0 | 1.34362366530036E−12 | 0.0781 |
| | | C18:1cis-9 | 6.1248248552359403E−8 | 0.1672 |
| 490 | 490 | C10:0 | 9.8960147737552498E−13 | 0.04697 |
| | | C12:0 | 4.7101571732416404E−12 | 0.05382 |
| | | C14:0 | 1.3301890805958101E−12 | 0.07811 |
| | | C18:1cis-9 | 6.0322589767257897E−8 | 0.1672 |
| 491 | 491 | C10:0 | 9.798085801963551E−13 | 0.04697 |
| | | C12:0 | 4.6635122971289602E−12 | 0.05382 |
| | | C14:0 | 1.2976574638717299E−12 | 0.07814 |
| | | C18:1cis-9 | 5.9112639767034598E−8 | 0.1673 |
| 492 | 492 | C10:0 | 9.4142521644103096E−13 | 0.04694 |
| | | C12:0 | 4.4588898500815303E−12 | 0.0538 |
| | | C14:0 | 1.1074265931101701E−12 | 0.07827 |
| | | C18:1cis-9 | 5.2598129988874097E−8 | 0.1677 |
| 493 | 493 | C10:0 | 9.4142521644103096E−13 | 0.04694 |
| | | C12:0 | 4.4588898500815303E−12 | 0.0538 |
| | | C14:0 | 1.1074265931101701E−12 | 0.07827 |
| | | C18:1cis-9 | 5.2598129988874097E−8 | 0.1677 |
| 494 | 494 | C10:0 | 9.4142521644103096E−13 | 0.04694 |
| | | C12:0 | 4.4588898500815303E−12 | 0.0538 |
| | | C14:0 | 1.1074265931101701E−12 | 0.07827 |
| | | C18:1cis-9 | 5.2598129988874097E−8 | 0.1677 |
| 495 | 495 | C6:0 | 8.6526872145536899E−13 | 0.03035 |
| | | C8:0 | 3.2087627978321099E−16 | 0.02791 |
| | | C12:0 | 8.8845096834888303E−11 | 0.07756 |
| 496 | 496 | C8:0 | 1.26331219870779E−15 | 0.02731 |
| 497 | 497 | C12:0 | 4.6053030477721603E−11 | 0.03385 |
| | | C14:0 | 7.0501025182482499E−14 | 0.0546 |
| | | C18:1cis-9 | 7.7836265611296704E−11 | 0.1328 |
| 498 | 498 | C8:0 | 8.1070756299992592E−15 | 0.02516 |
| 499 | 499 | C6:0 | 5.9695018587987398E−13 | 0.02775 |
| | | C8:0 | 9.3485919420065296E−15 | 0.02407 |
| 500 | 500 | C8:0 | 6.8938814238877397E−15 | 0.02504 |
| 501 | 501 | C6:0 | 6.46832445103233E−14 | 0.03208 |
| | | C8:0 | 6.4600902324189202E−18 | 0.02968 |
| | | C10:0 | 5.8193809226297705E−14 | 0.07652 |
| | | C12:0 | 4.5863400743583397E−12 | 0.08319 |
| | | C14:0 | 9.0540251767141702E−12 | 0.1159 |
| 502 | 502 | C8:0 | 1.96548709943932E−16 | 0.02974 |
| | | C10:0 | 4.3338353448316299E−13 | 0.0774 |
| | | C12:0 | 1.9534286026786999E−11 | 0.08476 |
| | | C14:0 | 7.0492835056981495E−11 | 0.1157 |
| 503 | 503 | C6:0 | 6.4678027041489594E−14 | 0.03211 |
| | | C8:0 | 6.4352786975035901E−18 | 0.02973 |
| | | C10:0 | 5.7618779673050795E−14 | 0.07672 |
| | | C12:0 | 4.3857342114073604E−12 | 0.08347 |
| | | C14:0 | 9.7064600229982607E−12 | 0.116 |
| 504 | 504 | C6:0 | 5.8370300122890094E−14 | 0.03208 |
| | | C8:0 | 5.9828049254068398E−18 | 0.02969 |
| | | C10:0 | 5.6777583013235299E−14 | 0.07656 |
| | | C12:0 | 4.4517957144493097E−12 | 0.08324 |
| | | C14:0 | 9.1445131272512596E−12 | 0.1158 |
| 505 | 505 | C6:0 | 5.8606259988554695E−14 | 0.03208 |
| | | C8:0 | 6.0119529741251203E−18 | 0.02969 |
| | | C10:0 | 5.7056605006040905E−14 | 0.07656 |
| | | C12:0 | 4.47387414896631E−12 | 0.08324 |
| | | C14:0 | 9.1900963229264497E−12 | 0.1158 |
| 506 | 506 | C6:0 | 5.8370300122890094E−14 | 0.03208 |
| | | C8:0 | 5.9828049254068398E−18 | 0.02969 |
| | | C10:0 | 5.6777583013235299E−14 | 0.07656 |
| | | C12:0 | 4.4517957144493097E−12 | 0.08324 |
| | | C14:0 | 9.1445131272512596E−12 | 0.1158 |
| 507 | 507 | C6:0 | 5.8370300122890094E−14 | 0.03208 |
| | | C8:0 | 5.9828049254068398E−18 | 0.02969 |
| | | C10:0 | 5.6777583013235299E−14 | 0.07656 |
| | | C12:0 | 4.4517957144493097E−12 | 0.08324 |
| | | C14:0 | 9.1445131272512596E−12 | 0.1158 |
| 508 | 508 | C6:0 | 5.2844296657701699E−14 | 0.03229 |
| | | C8:0 | 5.1216104581478098E−18 | 0.02988 |
| | | C10:0 | 4.8502395204224899E−14 | 0.07712 |
| | | C12:0 | 3.81704798800288E−12 | 0.08389 |
| | | C14:0 | 7.2037733862774998E−12 | 0.117 |
| 509 | 509 | C6:0 | 5.2844296657701699E−14 | 0.03229 |
| | | C8:0 | 5.1216104581478098E−18 | 0.02988 |
| | | C10:0 | 4.8502395204224899E−14 | 0.07712 |
| | | C12:0 | 3.81704798800288E−12 | 0.08389 |
| | | C14:0 | 7.2037733862774998E−12 | 0.117 |
| 510 | 510 | C6:0 | 2.8691885622826501E−14 | 0.03241 |
| | | C8:0 | 4.0955533857384502E−18 | 0.02978 |
| | | C10:0 | 6.4836699238860897E−14 | 0.07623 |
| | | C12:0 | 5.7624022031460201E−12 | 0.08265 |
| | | C14:0 | 1.08271402419137E−11 | 0.1152 |
| 511 | 511 | C6:0 | 8.6090132586570202E−14 | 0.03183 |
| | | C8:0 | 5.9828049254068398E−18 | 0.02965 |
| | | C10:0 | 4.0413783097584002E−14 | 0.0769 |
| | | C12:0 | 3.0526698792752799E−12 | 0.08376 |
| | | C14:0 | 6.3614861864064697E−12 | 0.1166 |
| 512 | 512 | C6:0 | 7.4954777718639699E−13 | 0.02986 |
| | | C8:0 | 1.1336634082662E−16 | 0.0278 |
| | | C10:0 | 3.2527348815077898E−13 | 0.07227 |
| | | C12:0 | 2.22798629292619E−11 | 0.07836 |
| | | C14:0 | 1.6683600965042399E−11 | 0.1113 |
| 513 | 513 | C6:0 | 7.4954777718639699E−13 | 0.02986 |
| | | C8:0 | 1.1336634082662E−16 | 0.0278 |
| | | C10:0 | 3.2527348815077898E−13 | 0.07227 |
| | | C12:0 | 2.22798629292619E−11 | 0.07836 |
| | | C14:0 | 1.6683600965042399E−11 | 0.1113 |
| 514 | 514 | C6:0 | 6.8958658606376797E−14 | 0.03187 |
| | | C8:0 | 4.8782858716258203E−18 | 0.02965 |
| | | C10:0 | 3.5393630579030497E−14 | 0.07687 |
| | | C12:0 | 2.6705116123192701E−12 | 0.08377 |
| | | C14:0 | 6.5879292893616699E−12 | 0.1162 |
| 515 | 515 | C8:0 | 2.9490947376025798E−15 | 0.02496 |
| | | C12:0 | 6.1729347068226006E−11 | 0.07231 |
| 516 | 516 | C8:0 | 3.6778283679910603E−15 | 0.0406 |
| 517 | 517 | C6:0 | 7.8777696618941104E−14 | 0.03187 |
| | | C8:0 | 5.4023514315186601E−18 | 0.02968 |
| | | C10:0 | 3.60900965378338E−14 | 0.07703 |
| | | C12:0 | 2.67015727068385E−12 | 0.08397 |
| | | C14:0 | 6.7194813785435399E−12 | 0.1164 |
| 518 | 518 | C6:0 | 7.8777696618941104E−14 | 0.03187 |
| | | C8:0 | 5.4023514315186601E−18 | 0.02968 |
| | | C10:0 | 3.60900965378338E−14 | 0.07703 |
| | | C12:0 | 2.67015727068385E−12 | 0.08397 |
| | | C14:0 | 6.7194813785435399E−12 | 0.1164 |
| 519 | 519 | C6:0 | 7.8777696618941104E−14 | 0.03187 |
| | | C8:0 | 5.4023514315186601E−18 | 0.02968 |
| | | C10:0 | 3.60900965378338E−14 | 0.07703 |
| | | C12:0 | 2.67015727068385E−12 | 0.08397 |
| | | C14:0 | 6.7194813785435399E−12 | 0.1164 |
| 520 | 520 | C6:0 | 7.8777696618941104E−14 | 0.03187 |
| | | C8:0 | 5.4023514315186601E−18 | 0.02968 |
| | | C10:0 | 3.60900965378338E−14 | 0.07703 |
| | | C12:0 | 2.67015727068385E−12 | 0.08397 |
| | | C14:0 | 6.7194813785435399E−12 | 0.1164 |
| 521 | 521 | C6:0 | 7.8777696618941104E−14 | 0.03187 |
| | | C8:0 | 5.4023514315186601E−18 | 0.02968 |
| | | C10:0 | 3.60900965378338E−14 | 0.07703 |
| | | C12:0 | 2.67015727068385E−12 | 0.08397 |
| | | C14:0 | 6.7194813785435399E−12 | 0.1164 |
| 522 | 522 | C6:0 | 7.8777696618941104E−14 | 0.03187 |
| | | C8:0 | 5.4023514315186601E−18 | 0.02968 |
| | | C10:0 | 3.60900965378338E−14 | 0.07703 |
| | | C12:0 | 2.67015727068385E−12 | 0.08397 |
| | | C14:0 | 6.7194813785435399E−12 | 0.1164 |
| 523 | 523 | C6:0 | 7.8777696618941104E−14 | 0.03187 |
| | | C8:0 | 5.4023514315186601E−18 | 0.02968 |
| | | C10:0 | 3.60900965378338E−14 | 0.07703 |
| | | C12:0 | 2.67015727068385E−12 | 0.08397 |
| | | C14:0 | 6.7194813785435399E−12 | 0.1164 |
| 524 | 524 | C6:0 | 7.8777696618941104E−14 | 0.03187 |
| | | C8:0 | 5.4023514315186601E−18 | 0.02968 |
| | | C10:0 | 3.60900965378338E−14 | 0.07703 |
| | | C12:0 | 2.67015727068385E−12 | 0.08397 |
| | | C14:0 | 6.7194813785435399E−12 | 0.1164 |
| 525 | 525 | C6:0 | 6.3731745223760804E−14 | 0.03183 |
| | | C8:0 | 5.5352228294095803E−18 | 0.02952 |
| | | C10:0 | 4.5014602845649898E−14 | 0.07634 |
| | | C12:0 | 3.40361060193182E−12 | 0.08313 |
| | | C14:0 | 8.6981209348386702E−12 | 0.1152 |
| 526 | 526 | C6:0 | 7.8777696618941104E−14 | 0.03187 |
| | | C8:0 | 5.4023514315186601E−18 | 0.02968 |
| | | C10:0 | 3.60900965378338E−14 | 0.07703 |
| | | C12:0 | 2.67015727068385E−12 | 0.08397 |
| | | C14:0 | 6.7194813785435399E−12 | 0.1164 |
| 527 | 527 | C6:0 | 7.8777696618941104E−14 | 0.03187 |
| | | C8:0 | 5.4023514315186601E−18 | 0.02968 |
| | | C10:0 | 3.60900965378338E−14 | 0.07703 |
| | | C12:0 | 2.67015727068385E−12 | 0.08397 |
| | | C14:0 | 6.7194813785435399E−12 | 0.1164 |
| 528 | 528 | C6:0 | 8.6099510700857397E−13 | 0.02985 |
| | | C8:0 | 1.2560358657241399E−16 | 0.02782 |
| | | C10:0 | 3.3337528695859998E−13 | 0.07241 |
| | | C12:0 | 2.2389699973462499E−11 | 0.07853 |
| | | C14:0 | 1.7103561141567702E−11 | 0.1115 |
| 529 | 529 | C6:0 | 7.8777696618941104E−14 | 0.03187 |
| | | C8:0 | 5.4023514315186601E−18 | 0.02968 |
| | | C10:0 | 3.60900965378338E−14 | 0.07703 |
| | | C12:0 | 2.67015727068385E−12 | 0.08397 |
| | | C14:0 | 6.7194813785435399E−12 | 0.1164 |
| 530 | 530 | C6:0 | 8.9997524897581702E−14 | 0.03185 |
| | | C8:0 | 6.0412432342423701E−18 | 0.02969 |
| | | C10:0 | 3.75352307596589E−14 | 0.0771 |
| | | C12:0 | 2.7099870413625398E−12 | 0.08408 |
| | | C14:0 | 6.9569812272975103E−12 | 0.1165 |
| 531 | 531 | C6:0 | 8.9997524897581702E−14 | 0.03185 |
| | | C8:0 | 6.0412432342423701E−18 | 0.02969 |
| | | C10:0 | 3.75352307596589E−14 | 0.0771 |
| | | C12:0 | 2.7099870413625398E−12 | 0.08408 |
| | | C14:0 | 6.9569812272975103E−12 | 0.1165 |
| 532 | 532 | C6:0 | 1.17069127353711E−13 | 0.03179 |
| | | C8:0 | 7.3378389392513199E−18 | 0.02966 |
| | | C10:0 | 4.1211431392182997E−14 | 0.0771 |
| | | C12:0 | 2.87594935447973E−12 | 0.08411 |
| | | C14:0 | 7.4577782254279306E−12 | 0.1165 |
| 533 | 533 | C6:0 | 1.17069127353711E−13 | 0.03179 |
| | | C8:0 | 7.3378389392513199E−18 | 0.02966 |
| | | C10:0 | 4.1211431392182997E−14 | 0.0771 |
| | | C12:0 | 2.87594935447973E−12 | 0.08411 |
| | | C14:0 | 7.4577782254279306E−12 | 0.1165 |
| 534 | 534 | C6:0 | 1.17069127353711E−13 | 0.03179 |
| | | C8:0 | 7.3378389392513199E−18 | 0.02966 |
| | | C10:0 | 4.1211431392182997E−14 | 0.0771 |
| | | C12:0 | 2.87594935447973E−12 | 0.08411 |
| | | C14:0 | 7.4577782254279306E−12 | 0.1165 |
| 535 | 535 | C6:0 | 1.17069127353711E−13 | 0.03179 |
| | | C8:0 | 7.3378389392513199E−18 | 0.02966 |
| | | C10:0 | 4.1211431392182997E−14 | 0.0771 |
| | | C12:0 | 2.87594935447973E−12 | 0.08411 |
| | | C14:0 | 7.4577782254279306E−12 | 0.1165 |
| 536 | 536 | C6:0 | 1.18050197534709E−13 | 0.03176 |
| | | C8:0 | 7.4819301094848397E−18 | 0.02967 |
| | | C10:0 | 4.02121462221512E−14 | 0.07717 |
| | | C12:0 | 2.7779431024913298E−12 | 0.08421 |
| | | C14:0 | 7.2751038856886499E−12 | 0.1166 |
| 537 | 537 | C6:0 | 9.7869401731918304E−14 | 0.03203 |
| | | C8:0 | 6.4986965004188899E−18 | 0.02987 |
| | | C10:0 | 3.9408520446929702E−14 | 0.07758 |
| | | C12:0 | 2.9474123618934201E−12 | 0.08452 |
| | | C14:0 | 7.4932481165886701E−12 | 0.1171 |
| 538 | 538 | C6:0 | 2.3103353192058902E−13 | 0.0314 |
| | | C8:0 | 1.51467923500645E−17 | 0.02941 |
| | | C10:0 | 5.8748065597100296E−14 | 0.0767 |
| | | C12:0 | 3.6310663124227601E−12 | 0.08379 |
| | | C14:0 | 9.1888404317268005E−12 | 0.1161 |
| 539 | 539 | C6:0 | 4.5583011817933699E−14 | 0.0321 |
| | | C8:0 | 3.7526867917927399E−18 | 0.02976 |
| | | C10:0 | 2.31583193419613E−14 | 0.07742 |
| | | C12:0 | 1.6498972566502901E−12 | 0.08455 |
| | | C14:0 | 4.4696569228457802E−12 | 0.1171 |
| 540 | 540 | C12:0 | 3.9320152099305201E−11 | 0.05508 |
| 541 | 541 | C6:0 | 7.3485275600098796E−13 | 0.03026 |
| | | C8:0 | 2.1501128207339601E−16 | 0.02804 |
| | | C10:0 | 4.64277210485625E−13 | 0.07274 |
| | | C12:0 | 2.95950043511028E−11 | 0.07888 |
| | | C14:0 | 2.0058022469162001E−11 | 0.1123 |
| 542 | 542 | C6:0 | 6.1189066506996495E−13 | 0.03033 |
| | | C8:0 | 1.37811661932649E−16 | 0.02805 |
| | | C10:0 | 4.4850896787682901E−13 | 0.07271 |
| | | C12:0 | 3.0041756692055502E−11 | 0.07878 |
| 543 | 543 | C8:0 | 6.2974756280658802E−15 | 0.01766 |
| | | C10:0 | 3.6772846519945002E−13 | 0.04857 |
| | | C12:0 | 5.2376115799734699E−12 | 0.05437 |
| | | C14:0 | 2.84669598414608E−12 | 0.07758 |
| | | C18:1cis-9 | 2.1778978217475099E−8 | 0.1767 |
| 544 | 544 | C6:0 | 2.9781681949215698E−15 | 0.0328 |
| | | C8:0 | 1.8205919140614801E−18 | 0.02936 |
| | | C10:0 | 5.29450458855621E−14 | 0.07462 |
| | | C12:0 | 4.2126395123090899E−12 | 0.08113 |
| | | C14:0 | 5.31729041193598E−12 | 0.114 |
| 545 | 545 | C6:0 | 1.9935163607514599E−14 | 0.03147 |
| | | C8:0 | 6.4075463746674299E−18 | 0.02855 |
| | | C10:0 | 8.9794236047964494E−14 | 0.07292 |
| | | C12:0 | 5.6939448076521802E−12 | 0.07965 |
| | | C14:0 | 1.19747216505542E−11 | 0.1101 |
| 546 | 546 | C6:0 | 6.1577210140203499E−15 | 0.03156 |
| | | C8:0 | 6.5935949195694402E−18 | 0.0281 |
| | | C10:0 | 1.4120329594138201E−13 | 0.07137 |
| | | C12:0 | 9.4198441244611398E−12 | 0.07764 |
| | | C14:0 | 9.1388972384758007E−12 | 0.1097 |
| 547 | 547 | C6:0 | 3.3175615415457701E−15 | 0.03213 |
| | | C8:0 | 2.4008482035435101E−18 | 0.02871 |
| | | C10:0 | 6.3831124638089105E−14 | 0.07298 |
| | | C12:0 | 4.76859011214647E−12 | 0.0794 |
| | | C14:0 | 5.4774797253576498E−12 | 0.1118 |
| 548 | 548 | C8:0 | 8.9441680841612995E−15 | 0.0224 |
| | | C14:0 | 7.7898404836716797E−11 | 0.09264 |
| 549 | 549 | C8:0 | 8.9441680841612995E−15 | 0.0224 |
| | | C14:0 | 7.7898404836716797E−11 | 0.09264 |
| 550 | 550 | C8:0 | 8.9441680841612995E−15 | 0.0224 |
| | | C14:0 | 7.7898404836716797E−11 | 0.09264 |
| 551 | 551 | C8:0 | 4.6302927506374902E−16 | 0.02468 |
| | | C10:0 | 5.8358489429137297E−13 | 0.06482 |
| | | C12:0 | 1.8359609669685801E−11 | 0.07134 |
| | | C14:0 | 1.74594372230884E−11 | 0.1009 |
| 552 | 552 | C14:0 | 8.9143996334826603E−11 | 0.09232 |
| 553 | 553 | C14:0 | 8.9143996334826603E−11 | 0.09232 |
| 554 | 554 | C6:0 | 1.98362859176475E−14 | 0.0311 |
| | | C8:0 | 8.2060250520263206E−18 | 0.02815 |
| | | C10:0 | 8.40747139441296E−14 | 0.07233 |
| | | C12:0 | 6.6494155920830596E−12 | 0.07852 |
| | | C14:0 | 3.6989178961073298E−12 | 0.1122 |
| 555 | 555 | C6:0 | 1.9836814556188401E−14 | 0.0311 |
| | | C8:0 | 8.0871943049434207E−18 | 0.02815 |
| | | C10:0 | 8.1621684319154994E−14 | 0.07235 |
| | | C12:0 | 6.4539668067409299E−12 | 0.07855 |
| | | C14:0 | 3.53729967674195E−12 | 0.1122 |
| 556 | 556 | C6:0 | 1.9836814556188401E−14 | 0.0311 |
| | | C8:0 | 8.0871943049434207E−18 | 0.02815 |
| | | C10:0 | 8.1621684319154994E−14 | 0.07235 |
| | | C12:0 | 6.4539668067409299E−12 | 0.07855 |
| | | C14:0 | 3.53729967674195E−12 | 0.1122 |
| 557 | 557 | C6:0 | 1.9836814556188401E−14 | 0.0311 |
| | | C8:0 | 8.0871943049434207E−18 | 0.02815 |
| | | C10:0 | 8.1621684319154994E−14 | 0.07235 |
| | | C12:0 | 6.4539668067409299E−12 | 0.07855 |
| | | C14:0 | 3.53729967674195E−12 | 0.1122 |
| 558 | 558 | C6:0 | 1.9836814556188401E−14 | 0.0311 |
| | | C8:0 | 8.0871943049434207E−18 | 0.02815 |
| | | C10:0 | 8.1621684319154994E−14 | 0.07235 |
| | | C12:0 | 6.4539668067409299E−12 | 0.07855 |
| | | C14:0 | 3.53729967674195E−12 | 0.1122 |
| 559 | 559 | C6:0 | 1.94523350166284E−14 | 0.0311 |
| | | C8:0 | 7.7033972493254596E−18 | 0.02816 |
| | | C10:0 | 7.7692815395340406E−14 | 0.0724 |
| | | C12:0 | 6.1716936497831503E−12 | 0.07861 |
| | | C14:0 | 3.39958755179364E−12 | 0.1123 |
| 560 | 560 | C6:0 | 4.6035529933010502E−14 | 0.02061 |
| | | C8:0 | 1.0759089800870299E−18 | 0.01951 |
| | | C10:0 | 7.5972957518285096E−16 | 0.0523 |
| | | C12:0 | 4.7934482954406602E−14 | 0.05769 |
| | | C14:0 | 5.1964277076951602E−14 | 0.08118 |
| 561 | 561 | C6:0 | 1.94523350166284E−14 | 0.0311 |
| | | C8:0 | 7.7033972493254596E−18 | 0.02816 |
| | | C10:0 | 7.7692815395340406E−14 | 0.0724 |
| | | C12:0 | 6.1716936497831503E−12 | 0.07861 |
| | | C14:0 | 3.39958755179364E−12 | 0.1123 |
| 562 | 562 | C6:0 | 1.94523350166284E−14 | 0.0311 |
| | | C8:0 | 7.7033972493254596E−18 | 0.02816 |
| | | C10:0 | 7.7692815395340406E−14 | 0.0724 |
| | | C12:0 | 6.1716936497831503E−12 | 0.07861 |
| | | C14:0 | 3.39958755179364E−12 | 0.1123 |
| 563 | 563 | C6:0 | 1.94523350166284E−14 | 0.0311 |
| | | C8:0 | 7.7033972493254596E−18 | 0.02816 |
| | | C10:0 | 7.7692815395340406E−14 | 0.0724 |
| | | C12:0 | 6.1716936497831503E−12 | 0.07861 |
| | | C14:0 | 3.39958755179364E−12 | 0.1123 |
| 564 | 564 | C6:0 | 1.94523350166284E−14 | 0.0311 |
| | | C8:0 | 7.7033972493254596E−18 | 0.02816 |
| | | C10:0 | 7.7692815395340406E−14 | 0.0724 |
| | | C12:0 | 6.1716936497831503E−12 | 0.07861 |
| | | C14:0 | 3.39958755179364E−12 | 0.1123 |
| 565 | 565 | C6:0 | 1.94523350166284E−14 | 0.0311 |
| | | C8:0 | 7.7033972493254596E−18 | 0.02816 |
| | | C10:0 | 7.7692815395340406E−14 | 0.0724 |
| | | C12:0 | 6.1716936497831503E−12 | 0.07861 |
| | | C14:0 | 3.39958755179364E−12 | 0.1123 |
| 566 | 566 | C6:0 | 2.0330308130571501E−14 | 0.03109 |
| | | C8:0 | 7.5183934897634105E−18 | 0.02818 |
| | | C10:0 | 7.1799581917588301E−14 | 0.07253 |
| | | C12:0 | 5.7283518442957199E−12 | 0.07876 |
| | | C14:0 | 3.1089957748248099E−12 | 0.1125 |
| 567 | 567 | C6:0 | 4.1715114945833403E−14 | 0.03076 |
| | | C8:0 | 1.8402369977282401E−17 | 0.02788 |
| | | C10:0 | 1.49738523552676E−13 | 0.07168 |
| | | C12:0 | 1.12072025696161E−11 | 0.07777 |
| | | C14:0 | 6.4836206798199E−12 | 0.111 |
| 568 | 568 | C6:0 | 2.0330308130571501E−14 | 0.03109 |
| | | C8:0 | 7.5183934897634105E−18 | 0.02818 |
| | | C10:0 | 7.1799581917588301E−14 | 0.07253 |
| | | C12:0 | 5.7283518442957199E−12 | 0.07876 |
| | | C14:0 | 3.1089957748248099E−12 | 0.1125 |
| 569 | 569 | C6:0 | 2.0330308130571501E−14 | 0.03109 |
| | | C8:0 | 7.5183934897634105E−18 | 0.02818 |
| | | C10:0 | 7.1799581917588301E−14 | 0.07253 |
| | | C12:0 | 5.7283518442957199E−12 | 0.07876 |
| | | C14:0 | 3.1089957748248099E−12 | 0.1125 |
| 570 | 570 | C6:0 | 2.0330308130571501E−14 | 0.03109 |
| | | C8:0 | 7.5183934897634105E−18 | 0.02818 |
| | | C10:0 | 7.1799581917588301E−14 | 0.07253 |
| | | C12:0 | 5.7283518442957199E−12 | 0.07876 |
| | | C14:0 | 3.1089957748248099E−12 | 0.1125 |
| 571 | 571 | C6:0 | 1.5515552482962701E−15 | 0.01787 |
| | | C8:0 | 5.8390249193305097E−17 | 0.01513 |
| | | C10:0 | 6.6990538012810396E−13 | 0.03851 |
| | | C12:0 | 8.9153993573030205E−11 | 0.04103 |
| | | C14:0 | 1.97577389061098E−11 | 0.05987 |
| 572 | 572 | C6:0 | 2.3104976993783701E−14 | 0.03115 |
| | | C8:0 | 6.7887314298445697E−18 | 0.02833 |
| | | C10:0 | 5.7239491626225202E−14 | 0.07312 |
| | | C12:0 | 4.6964631674301602E−12 | 0.07941 |
| | | C14:0 | 2.37837536205307E−12 | 0.1136 |
| 573 | 573 | C6:0 | 2.3104976993783701E−14 | 0.03115 |
| | | C8:0 | 6.7887314298445697E−18 | 0.02833 |
| | | C10:0 | 5.7239491626225202E−14 | 0.07312 |
| | | C12:0 | 4.6964631674301602E−12 | 0.07941 |
| | | C14:0 | 2.37837536205307E−12 | 0.1136 |
| 574 | 574 | C6:0 | 2.3104976993783701E−14 | 0.03115 |
| | | C8:0 | 6.7887314298445697E−18 | 0.02833 |
| | | C10:0 | 5.7239491626225202E−14 | 0.07312 |
| | | C12:0 | 4.6964631674301602E−12 | 0.07941 |
| | | C14:0 | 2.37837536205307E−12 | 0.1136 |
| 575 | 575 | C6:0 | 2.3104976993783701E−14 | 0.03115 |
| | | C8:0 | 6.7887314298445697E−18 | 0.02833 |
| | | C10:0 | 5.7239491626225202E−14 | 0.07312 |
| | | C12:0 | 4.6964631674301602E−12 | 0.07941 |
| | | C14:0 | 2.37837536205307E−12 | 0.1136 |
| 576 | 576 | C6:0 | 2.3104976993783701E−14 | 0.03115 |
| | | C8:0 | 6.7887314298445697E−18 | 0.02833 |
| | | C10:0 | 5.7239491626225202E−14 | 0.07312 |
| | | C12:0 | 4.6964631674301602E−12 | 0.07941 |
| | | C14:0 | 2.37837536205307E−12 | 0.1136 |
| 577 | 577 | C6:0 | 1.11813746694359E−13 | 0.03043 |
| | | C8:0 | 4.3583673414414098E−17 | 0.02773 |
| | | C10:0 | 2.3241356856986502E−13 | 0.07157 |
| | | C12:0 | 1.5800231331830499E−11 | 0.0777 |
| | | C14:0 | 6.7806708187726702E−12 | 0.1116 |
| 578 | 578 | C6:0 | 5.1094854026620902E−15 | 0.0174 |
| | | C8:0 | 1.86730902209013E−17 | 0.01524 |
| | | C10:0 | 1.01891692801114E−13 | 0.0395 |
| | | C12:0 | 1.4659211231729599E−11 | 0.04232 |
| | | C14:0 | 3.6985796833596601E−12 | 0.06142 |
| 579 | 579 | C6:0 | 3.1974097320826697E−14 | 0.03109 |
| | | C8:0 | 7.09233371815551E−18 | 0.02841 |
| | | C10:0 | 4.81821658986466E−14 | 0.07359 |
| | | C12:0 | 3.9870678737670197E−12 | 0.07995 |
| | | C14:0 | 1.9798379235384702E−12 | 0.1145 |
| 580 | 580 | C6:0 | 5.2326051203402503E−14 | 0.03085 |
| | | C8:0 | 1.1422488315846599E−17 | 0.02825 |
| | | C10:0 | 7.2930274343917101E−14 | 0.07313 |
| | | C12:0 | 5.9934868100761697E−12 | 0.07937 |
| | | C14:0 | 2.65358421316355E−12 | 0.1139 |
| 581 | 581 | C6:0 | 9.2737948003598496E−13 | 0.03034 |
| | | C8:0 | 1.83293017350549E−15 | 0.02722 |
| 582 | 582 | C6:0 | 8.5251120766593496E−13 | 0.0304 |
| | | C8:0 | 1.8149939038893202E−15 | 0.02724 |
| 583 | 583 | C6:0 | 8.6956358601963199E−13 | 0.03043 |
| | | C8:0 | 1.6863492757359999E−15 | 0.02731 |
| 584 | 584 | C6:0 | 1.5640280767144001E−13 | 0.03117 |
| | | C8:0 | 2.4049504936820502E−16 | 0.0279 |
| | | C14:0 | 7.9905304249101799E−11 | 0.1093 |
| 585 | 585 | C6:0 | 3.6882234002487498E−14 | 0.03257 |
| | | C8:0 | 1.8383449209801899E−18 | 0.03035 |
| | | C10:0 | 1.36924786745353E−14 | 0.07896 |
| | | C12:0 | 9.7634820058186092E−13 | 0.08637 |
| | | C14:0 | 1.72486244075151E−12 | 0.1207 |
| 586 | 586 | C8:0 | 1.5140034590119901E−15 | 0.02442 |
| | | C10:0 | 3.3831081182036098E−13 | 0.06588 |
| | | C12:0 | 7.5666702304575396E−12 | 0.07313 |
| | | C14:0 | 6.2309245299310697E−12 | 0.1036 |
| 587 | 587 | C14:0 | 9.3056726983825702E−11 | 0.06549 |
| 588 | 588 | C12:0 | 3.45891155467598E−11 | 0.04769 |
| | | C14:0 | 1.39249980298512E−12 | 0.07174 |
| | | C18:1cis-9 | 2.6283604811214599E−8 | 0.1606 |
| 589 | 589 | C10:0 | 3.1143358297115698E−13 | 0.0447 |
| | | C12:0 | 5.4053323750460298E−12 | 0.04984 |
| | | C14:0 | 1.1641333915061901E−13 | 0.07546 |
| | | C18:1cis-9 | 3.9350672508404297E−9 | 0.1706 |
| 590 | 590 | C6:0 | 6.6291988274311306E−14 | 0.03217 |
| | | C8:0 | 3.7528511457224296E−18 | 0.03001 |
| | | C10:0 | 1.5256093268115298E−14 | 0.07863 |
| | | C12:0 | 8.4994339979418203E−13 | 0.08639 |
| | | C14:0 | 8.7499116323703404E−13 | 0.122 |
| 591 | 591 | C6:0 | 6.6291988274311306E−14 | 0.03217 |
| | | C8:0 | 3.7528511457224296E−18 | 0.03001 |
| | | C10:0 | 1.5256093268115298E−14 | 0.07863 |
| | | C12:0 | 8.4994339979418203E−13 | 0.08639 |
| | | C14:0 | 8.7499116323703404E−13 | 0.122 |
| 592 | 592 | C6:0 | 3.39681306090115E−13 | 0.03129 |
| | | C8:0 | 4.0116611887275599E−17 | 0.02912 |
| | | C10:0 | 1.08848446213586E−13 | 0.07616 |
| | | C12:0 | 4.6324517499887898E−12 | 0.0837 |
| | | C14:0 | 5.0126633891006798E−12 | 0.118 |
| 593 | 593 | C8:0 | 2.18105714910406E−16 | 0.02826 |
| | | C10:0 | 4.5123976060765801E−13 | 0.07377 |
| | | C12:0 | 1.6130877182238299E−11 | 0.08104 |
| | | C14:0 | 1.7030748351964E−11 | 0.1143 |
| 594 | 594 | C8:0 | 3.9585123328324296E−15 | 0.02648 |
| | | C12:0 | 5.3891410098447302E−11 | 0.07723 |
| | | C14:0 | 3.7992610279941E−11 | 0.1099 |
| 595 | 595 | C4:0 | 1.1618660950668801E−7 | 0.02482 |
| | | C6:0 | 1.87506150599083E−5 | 0.01168 |
| 596 | 596 | C4:0 | 8.5775026528943598E−10 | 0.04225 |
| | | C6:0 | 2.4443641788527101E−5 | 0.01692 |
| 597 | 597 | C4:0 | 2.8770112674904698E−7 | 0.02885 |
| | | C6:0 | 1.9727754006559E−7 | 0.01701 |
| 598 | 598 | C4:0 | 5.2592132122375695E−7 | 0.03631 |
| 599 | 599 | C4:0 | 8.3230409248311496E−10 | 0.04228 |
| | | C6:0 | 2.3940221412394202E−5 | 0.01694 |
| 600 | 600 | C4:0 | 2.86237147958134E−7 | 0.02886 |
| | | C6:0 | 1.9727754006559E−7 | 0.01701 |
| 601 | 601 | C4:0 | 1.24382885819805E−9 | 0.04174 |
| | | C6:0 | 3.1712579023418199E−5 | 0.01665 |
| 602 | 602 | C4:0 | 1.15355196196173E−9 | 0.04183 |
| | | C6:0 | 3.1220701089658397E−5 | 0.01666 |
| 603 | 603 | C6:0 | 1.4767300384339199E−5 | 0.05324 |
| 604 | 604 | C4:0 | 1.74611849994922E−7 | 0.03013 |
| | | C6:0 | 1.6590699704015801E−7 | 0.01755 |
| 605 | 605 | C4:0 | 9.1602659442445695E−10 | 0.04152 |
| | | C6:0 | 3.2890832433734701E−5 | 0.01639 |
| 606 | 606 | C4:0 | 2.5073060684692002E−7 | 0.02976 |
| | | C6:0 | 2.9064348122720599E−7 | 0.01721 |
| 607 | 607 | C4:0 | 1.5452471254047201E−7 | 0.03026 |
| | | C6:0 | 1.90370428670056E−7 | 0.01747 |
| 608 | 608 | C4:0 | 1.8187351847701201E−7 | 0.03008 |
| | | C6:0 | 1.71927844759767E−7 | 0.01752 |
| 609 | 609 | C6:0 | 1.4767300384339199E−5 | 0.05324 |
| 610 | 610 | C6:0 | 1.4767300384339199E−5 | 0.05324 |
| 611 | 611 | C4:0 | 1.88475630137757E−7 | 0.03004 |
| | | C6:0 | 1.7999328304956801E−7 | 0.01749 |
| 612 | 612 | C6:0 | 6.9930642700733294E−5 | 0.01813 |
| 613 | 613 | C6:0 | 1.4767300384339199E−5 | 0.05324 |
| 614 | 614 | C6:0 | 1.4767300384339199E−5 | 0.05324 |
| 615 | 615 | C4:0 | 4.3319641311340998E−7 | 0.0361 |
| 616 | 616 | C4:0 | 4.3319641311340998E−7 | 0.0361 |
| 617 | 617 | C6:0 | 1.4767300384339199E−5 | 0.05324 |
| 618 | 618 | C6:0 | 1.4767300384339199E−5 | 0.05324 |
| 619 | 619 | C6:0 | 1.4767300384339199E−5 | 0.05324 |
| 620 | 620 | C6:0 | 1.4767300384339199E−5 | 0.05324 |
| 621 | 621 | C6:0 | 1.4767300384339199E−5 | 0.05324 |
| 622 | 622 | C4:0 | 3.3102592682480099E−7 | 0.0348 |
| 623 | 623 | C4:0 | 1.00282241869483E−7 | 0.03053 |
| | | C6:0 | 2.4944854469524201E−7 | 0.01746 |
| 624 | 624 | C4:0 | 1.01307092549129E−7 | 0.03052 |
| | | C6:0 | 1.62561961846854E−7 | 0.01746 |
| 625 | 625 | C4:0 | 3.47572458817078E−7 | 0.02948 |
| | | C6:0 | 8.9951404636187296E−7 | 0.01653 |
| 626 | 626 | C6:0 | 2.9024820328687102E−5 | 0.04755 |
| 627 | 627 | C4:0 | 9.92548073128146E−8 | 0.02642 |
| | | C6:0 | 2.39436512069864E−7 | 0.0149 |
| 628 | 628 | C6:0 | 8.0555988387369701E−6 | 0.01203 |
| | | C8:0 | 1.1563117021960201E−6 | 0.01057 |
| | | C10:0 | 6.0606651550769701E−6 | 0.02945 |
| 629 | 629 | C4:0 | 9.5785004332955504E−8 | 0.02645 |
| | | C6:0 | 2.05485444983831E−7 | 0.01498 |
| 630 | 630 | C6:0 | 3.0897053714715597E−5 | 0.04728 |
| 631 | 631 | C6:0 | 3.0897053714715597E−5 | 0.04728 |
| 632 | 632 | C6:0 | 3.0897053714715597E−5 | 0.04728 |
| 633 | 633 | C4:0 | 1.08126790372323E−9 | 0.0411 |
| | | C6:0 | 2.10216527171677E−5 | 0.01669 |
| 634 | 634 | C4:0 | 1.08126790372323E−9 | 0.0411 |
| | | C6:0 | 2.10216527171677E−5 | 0.01669 |
| 635 | 635 | C4:0 | 1.08126790372323E−9 | 0.0411 |
| | | C6:0 | 2.10216527171677E−5 | 0.01669 |
| 636 | 636 | C4:0 | 1.08671142852652E−9 | 0.04109 |
| | | C6:0 | 2.11312165605433E−5 | 0.01668 |
| 637 | 637 | C4:0 | 2.9576637385185398E−9 | 0.03848 |
| | | C6:0 | 1.63824025137785E−5 | 0.01625 |
| 638 | 638 | C4:0 | 1.0231515461465901E−9 | 0.04116 |
| | | C6:0 | 2.0696377017675298E−5 | 0.0167 |
| 639 | 639 | C4:0 | 1.0544456822863601E−9 | 0.04113 |
| | | C6:0 | 2.0165458950355998E−5 | 0.01672 |
| 640 | 640 | C4:0 | 1.0544456822863601E−9 | 0.04113 |
| | | C6:0 | 2.0165458950355998E−5 | 0.01672 |
| 641 | 641 | C4:0 | 1.07046270940047E−9 | 0.04112 |
| | | C6:0 | 2.0589250985754599E−5 | 0.01671 |
| 642 | 642 | C4:0 | 2.0784675981577999E−9 | 0.0407 |
| | | C6:0 | 3.5381161771517703E−5 | 0.01634 |
| 643 | 643 | C4:0 | 1.8237336796506801E−9 | 0.04085 |
| | | C6:0 | 3.6888517435643801E−5 | 0.01631 |
| 644 | 644 | C4:0 | 1.8237336796506801E−9 | 0.04085 |
| | | C6:0 | 3.6888517435643801E−5 | 0.01631 |
| 645 | 645 | C4:0 | 1.0544456822863601E−9 | 0.04113 |
| | | C6:0 | 2.0165458950355998E−5 | 0.01672 |
| 646 | 646 | C4:0 | 1.0544456822863601E−9 | 0.04113 |
| | | C6:0 | 2.0270535452615499E−5 | 0.01672 |
| 647 | 647 | C4:0 | 1.0544456822863601E−9 | 0.04113 |
| | | C6:0 | 2.0165458950355998E−5 | 0.01672 |
| 648 | 648 | C4:0 | 8.0938078560925399E−11 | 0.04558 |
| | | C6:0 | 4.55501839917025E−7 | 0.02056 |
| 649 | 649 | C4:0 | 1.0544456822863601E−9 | 0.04113 |
| | | C6:0 | 2.0165458950355998E−5 | 0.01672 |
| 650 | 650 | C4:0 | 1.0544456822863601E−9 | 0.04113 |
| | | C6:0 | 2.0165458950355998E−5 | 0.01672 |
| 651 | 651 | C4:0 | 1.0544456822863601E−9 | 0.04113 |
| | | C6:0 | 2.0165458950355998E−5 | 0.01672 |
| 652 | 652 | C4:0 | 1.01809514887765E−7 | 0.02637 |
| | | C6:0 | 2.4436979963224998E−7 | 0.01487 |
| 653 | 653 | C4:0 | 2.21736477708823E−8 | 0.04184 |
| 654 | 654 | C4:0 | 1.0544456822863601E−9 | 0.04113 |
| | | C6:0 | 2.0165458950355998E−5 | 0.01672 |
| 655 | 655 | C4:0 | 9.9291151836065809E−10 | 0.04144 |
| | | C6:0 | 1.97506018238042E−5 | 0.01684 |
| 656 | 656 | C4:0 | 3.2951542250039699E−10 | 0.04151 |
| | | C6:0 | 2.9481899757977401E−5 | 0.01606 |
| 657 | 657 | C4:0 | 1.89032228314557E−10 | 0.04193 |
| | | C6:0 | 1.66579155899724E−5 | 0.0165 |
| 658 | 658 | C4:0 | 1.89032228314557E−10 | 0.04193 |
| | | C6:0 | 1.66579155899724E−5 | 0.0165 |
| 659 | 659 | C4:0 | 4.12878830005107E−10 | 0.04141 |
| | | C6:0 | 1.7435555767282701E−5 | 0.01657 |
| 660 | 660 | C4:0 | 1.2635073090562301E−9 | 0.04101 |
| | | C6:0 | 2.4828183205161101E−5 | 0.01657 |
| 661 | 661 | C4:0 | 1.8606853132403501E−9 | 0.04066 |
| | | C6:0 | 3.6505725452266703E−5 | 0.01625 |
| 662 | 662 | C4:0 | 1.8606853132403501E−9 | 0.04066 |
| | | C6:0 | 3.6505725452266703E−5 | 0.01625 |
| 663 | 663 | C4:0 | 1.8606853132403501E−9 | 0.04066 |
| | | C6:0 | 3.6505725452266703E−5 | 0.01625 |
| 664 | 664 | C4:0 | 1.8606853132403501E−9 | 0.04066 |
| | | C6:0 | 3.6505725452266703E−5 | 0.01625 |
| 665 | 665 | C4:0 | 5.3957665177536797E−8 | 0.02693 |
| | | C6:0 | 2.0245348381075399E−7 | 0.01498 |
| 666 | 666 | C4:0 | 1.0544456822863601E−9 | 0.04113 |
| | | C6:0 | 2.0165458950355998E−5 | 0.01672 |
| 667 | 667 | C4:0 | 1.0544456822863601E−9 | 0.04113 |
| | | C6:0 | 2.0165458950355998E−5 | 0.01672 |
| 668 | 668 | C6:0 | 3.18783454582949E−5 | 0.0472 |
| 669 | 669 | C4:0 | 1.0651007654582601E−9 | 0.04113 |
| | | C6:0 | 2.08355182461092E−5 | 0.01671 |
| 670 | 670 | C4:0 | 4.3070407070282199E−7 | 0.03019 |
| 671 | 671 | C4:0 | 1.9140576289726499E−7 | 0.03065 |
| 672 | 672 | C4:0 | 1.0544456822863601E−9 | 0.04113 |
| | | C6:0 | 2.0165458950355998E−5 | 0.01672 |
| 673 | 673 | C4:0 | 1.05498276438438E−7 | 0.02634 |
| | | C6:0 | 2.35802231722049E−7 | 0.01489 |
| 674 | 674 | C4:0 | 1.05498276438438E−7 | 0.02634 |
| | | C6:0 | 2.35802231722049E−7 | 0.01489 |
| 675 | 675 | C4:0 | 4.4667754205104502E−7 | 0.03611 |
| 676 | 676 | C4:0 | 1.05498276438438E−7 | 0.02634 |
| | | C6:0 | 2.35802231722049E−7 | 0.01489 |
| 677 | 677 | C4:0 | 3.0267147342798303E−8 | 0.02571 |
| | | C6:0 | 2.1622889193497601E−7 | 0.01401 |
| 678 | 678 | C6:0 | 2.78405600516989E−5 | 0.04777 |
| 679 | 679 | C4:0 | 4.2226348309097701E−7 | 0.03619 |
| 680 | 680 | C4:0 | 4.20112169499275E−7 | 0.03619 |
| 681 | 681 | C4:0 | 2.48431465395573E−8 | 0.0258 |
| | | C6:0 | 2.4436979963224998E−7 | 0.01391 |
| 682 | 682 | C6:0 | 1.0382905138351801E−6 | 0.01187 |
| | | C8:0 | 7.0528001767501699E−5 | 0.00780 |
| 683 | 683 | C8:0 | 1.3128300867654499E−6 | 0.01271 |
| 684 | 684 | C4:0 | 8.9298821335757596E−5 | 0.01577 |
| 685 | 685 | C4:0 | 8.9298821335757596E−5 | 0.01577 |
| 686 | 686 | C4:0 | 6.5548266959015295E−5 | 0.01605 |
| 687 | 687 | C4:0 | 6.5889625302977805E−5 | 0.016 |
| 688 | 688 | C6:0 | 4.3842418759943598E−6 | 0.01004 |
| 689 | 689 | C4:0 | 1.16228820731041E−6 | 0.01883 |
| | | C6:0 | 8.7055486840779193E−6 | 0.01002 |
| 690 | 690 | C4:0 | 1.16228820731041E−6 | 0.01883 |
| | | C6:0 | 8.7055486840779193E−6 | 0.01002 |
| 691 | 691 | C4:0 | 1.03282877090213E−5 | 0.01701 |
| | | C6:0 | 1.02247149082522E−6 | 0.01098 |
| 692 | 692 | C4:0 | 6.7450857480694106E−8 | 0.02206 |
| | | C6:0 | 5.8603618002275503E−9 | 0.01364 |
| 693 | 693 | C4:0 | 1.15042935283043E−6 | 0.01886 |
| | | C6:0 | 4.4805552511256197E−5 | 0.00999 |
| 694 | 694 | C4:0 | 1.15042935283043E−6 | 0.01886 |
| | | C6:0 | 4.4805552511256197E−5 | 0.00999 |
| 695 | 695 | C4:0 | 2.0338431734047502E−6 | 0.01856 |
| | | C6:0 | 1.5313518234136699E−5 | 0.00984 |
| 696 | 696 | C4:0 | 1.1155663194914399E−6 | 0.01887 |
| | | C6:0 | 7.4543968075378499E−6 | 0.01011 |
| 697 | 697 | C4:0 | 1.15042935283043E−6 | 0.01886 |
| | | C6:0 | 4.4805552511256197E−5 | 0.00999 |
| 698 | 698 | C4:0 | 3.18605912710819E−7 | 0.02 |
| | | C6:0 | 1.4240667177477399E−5 | 0.00990 |
| 699 | 699 | C4:0 | 4.9685390313044501E−7 | 0.01971 |
| | | C6:0 | 1.2712107498138099E−6 | 0.01096 |
| 700 | 700 | C4:0 | 4.9685390313044501E−7 | 0.01971 |
| | | C6:0 | 1.2712107498138099E−6 | 0.01096 |
| 701 | 701 | C4:0 | 4.9685390313044501E−7 | 0.01971 |
| | | C6:0 | 1.2712107498138099E−6 | 0.01096 |
| 702 | 702 | C4:0 | 4.9685390313044501E−7 | 0.01971 |
| | | C6:0 | 1.2712107498138099E−6 | 0.01096 |
| 703 | 703 | C4:0 | 4.9685390313044501E−7 | 0.01971 |
| | | C6:0 | 1.2712107498138099E−6 | 0.01096 |
| 704 | 704 | C4:0 | 4.9685390313044501E−7 | 0.01971 |
| | | C6:0 | 1.2712107498138099E−6 | 0.01096 |
| 705 | 705 | C4:0 | 4.9685390313044501E−7 | 0.01971 |
| | | C6:0 | 1.2712107498138099E−6 | 0.01096 |
| 706 | 706 | C4:0 | 4.9685390313044501E−7 | 0.01971 |
| | | C6:0 | 1.2712107498138099E−6 | 0.01096 |
| 707 | 707 | C4:0 | 1.3742690993513E−7 | 0.02065 |
| | | C6:0 | 1.7435555767282701E−5 | 0.00981 |
| 708 | 708 | C4:0 | 3.4046304440987299E−7 | 0.01998 |
| | | C6:0 | 2.11312165605433E−5 | 0.00971 |
| 709 | 709 | C4:0 | 4.9685390313044501E−7 | 0.01971 |
| | | C6:0 | 1.2712107498138099E−6 | 0.01096 |
| 710 | 710 | C4:0 | 4.9685390313044501E−7 | 0.01971 |
| | | C6:0 | 1.2712107498138099E−6 | 0.01096 |
| 711 | 711 | C4:0 | 3.4220541746175402E−7 | 0.01998 |
| | | C6:0 | 2.0815183591539699E−5 | 0.00972 |
| 712 | 712 | C4:0 | 4.9685390313044501E−7 | 0.01971 |
| | | C6:0 | 1.2712107498138099E−6 | 0.01096 |
| 713 | 713 | C4:0 | 4.9685390313044501E−7 | 0.01971 |
| | | C6:0 | 1.2712107498138099E−6 | 0.01096 |
| 714 | 714 | C4:0 | 4.9685390313044501E−7 | 0.01971 |
| | | C6:0 | 1.2712107498138099E−6 | 0.01096 |
| 715 | 715 | C4:0 | 4.9685390313044501E−7 | 0.01971 |
| | | C6:0 | 1.2712107498138099E−6 | 0.01096 |
| 716 | 716 | C4:0 | 4.9685390313044501E−7 | 0.01971 |
| | | C6:0 | 1.2712107498138099E−6 | 0.01096 |
| 717 | 717 | C4:0 | 4.9685390313044501E−7 | 0.01971 |
| | | C6:0 | 1.2712107498138099E−6 | 0.01096 |
| 718 | 718 | C4:0 | 4.9685390313044501E−7 | 0.01971 |
| | | C6:0 | 1.2712107498138099E−6 | 0.01096 |
| 719 | 719 | C4:0 | 9.5656515974022401E−7 | 0.01906 |
| | | C6:0 | 5.60227624475701E−5 | 0.00913 |
| 720 | 720 | C4:0 | 5.9119092743757697E−7 | 0.01957 |
| | | C6:0 | 2.9036124641835601E−5 | 0.00955 |
| 721 | 721 | C4:0 | 2.08045225636637E−5 | 0.01623 |
| | | C6:0 | 5.1504182374085899E−5 | 0.00903 |
| 722 | 722 | C4:0 | 2.08045225636637E−5 | 0.01623 |
| | | C6:0 | 5.1504182374085899E−5 | 0.00903 |
| 723 | 723 | C4:0 | 9.1207647537005999E−6 | 0.01689 |
| | | C6:0 | 5.3725346623991897E−5 | 0.00896 |
| 724 | 724 | C4:0 | 9.1207647537005999E−6 | 0.01689 |
| | | C6:0 | 5.3725346623991897E−5 | 0.00896 |
| 725 | 725 | C4:0 | 9.1207647537005999E−6 | 0.01689 |
| | | C6:0 | 5.3725346623991897E−5 | 0.00896 |
| 726 | 726 | C4:0 | 2.08045225636637E−5 | 0.01623 |
| | | C6:0 | 5.1504182374085899E−5 | 0.00903 |
| 727 | 727 | C4:0 | 2.08045225636637E−5 | 0.01623 |
| | | C6:0 | 5.1504182374085899E−5 | 0.00903 |
| 728 | 728 | C4:0 | 2.08045225636637E−5 | 0.01623 |
| | | C6:0 | 4.9934153759076597E−5 | 0.00901 |
| 729 | 729 | C4:0 | 2.9025268849138001E−5 | 0.01597 |
| | | C6:0 | 4.6899530677258403E−5 | 0.00905 |
| 730 | 730 | C4:0 | 2.1241646155138502E−5 | 0.01621 |
| | | C6:0 | 4.7640771250574599E−5 | 0.00903 |
| 731 | 731 | C4:0 | 2.1241646155138502E−5 | 0.01621 |
| | | C6:0 | 4.7640771250574599E−5 | 0.00903 |
| 732 | 732 | C4:0 | 2.1241646155138502E−5 | 0.01621 |
| | | C6:0 | 4.7640771250574599E−5 | 0.00903 |
| 733 | 733 | C4:0 | 2.1241646155138502E−5 | 0.01621 |
| | | C6:0 | 4.7640771250574599E−5 | 0.00903 |
| 734 | 734 | C4:0 | 2.0912947997407001E−5 | 0.01622 |
| | | C6:0 | 4.7392096983275499E−5 | 0.00903 |
| 735 | 735 | C4:0 | 2.1241646155138502E−5 | 0.01621 |
| | | C6:0 | 4.7640771250574599E−5 | 0.00903 |
| 736 | 736 | C4:0 | 1.36621221873764E−5 | 0.0166 |
| | | C6:0 | 3.3935934612473601E−5 | 0.00921 |
| 737 | 737 | C4:0 | 1.36621221873764E−5 | 0.0166 |
| | | C6:0 | 3.3935934612473601E−5 | 0.00921 |
| 738 | 738 | C4:0 | 1.36621221873764E−5 | 0.0166 |
| | | C6:0 | 3.3935934612473601E−5 | 0.00921 |
| 739 | 739 | C4:0 | 1.36621221873764E−5 | 0.0166 |
| | | C6:0 | 3.3935934612473601E−5 | 0.00921 |
| 740 | 740 | C4:0 | 1.36621221873764E−5 | 0.0166 |
| | | C6:0 | 3.3935934612473601E−5 | 0.00921 |
| 741 | 741 | C4:0 | 2.1241646155138502E−5 | 0.01621 |
| | | C6:0 | 4.7640771250574599E−5 | 0.00903 |
| 742 | 742 | C4:0 | 2.1463718783526501E−5 | 0.01621 |
| | | C6:0 | 4.6655400362015797E−5 | 0.00904 |
| 743 | 743 | C4:0 | 2.1463718783526501E−5 | 0.01621 |
| | | C6:0 | 4.6655400362015797E−5 | 0.00904 |
| 744 | 744 | C4:0 | 2.1463718783526501E−5 | 0.01621 |
| | | C6:0 | 4.6655400362015797E−5 | 0.00904 |
| 745 | 745 | C4:0 | 6.6936075540827497E−5 | 0.01529 |
| 746 | 746 | C4:0 | 2.13523628740137E−5 | 0.01621 |
| | | C6:0 | 4.8140727121426502E−5 | 0.00903 |
| 747 | 747 | C4:0 | 2.08045225636637E−5 | 0.01624 |
| | | C6:0 | 4.6412549569670603E−5 | 0.00905 |
| 748 | 748 | C4:0 | 2.04826263644331E−5 | 0.01625 |
| | | C6:0 | 4.3139992759751598E−5 | 0.00909 |
| 749 | 749 | C4:0 | 2.1241646155138502E−5 | 0.01621 |
| | | C6:0 | 4.7640771250574599E−5 | 0.00903 |
| 750 | 750 | C4:0 | 2.1241646155138502E−5 | 0.01621 |
| | | C6:0 | 4.7640771250574599E−5 | 0.00903 |
| 751 | 751 | C4:0 | 2.1241646155138502E−5 | 0.01621 |
| | | C6:0 | 4.7640771250574599E−5 | 0.00903 |
| 752 | 752 | C4:0 | 2.1241646155138502E−5 | 0.01621 |
| | | C6:0 | 4.7640771250574599E−5 | 0.00903 |
| 753 | 753 | C4:0 | 2.1241646155138502E−5 | 0.01621 |
| | | C6:0 | 4.7640771250574599E−5 | 0.00903 |
| 754 | 754 | C4:0 | 2.1241646155138502E−5 | 0.01621 |
| | | C6:0 | 4.7640771250574599E−5 | 0.00903 |
| 755 | 755 | C4:0 | 2.1241646155138502E−5 | 0.01621 |
| | | C6:0 | 4.7640771250574599E−5 | 0.00903 |
| 756 | 756 | C4:0 | 2.08045225636637E−5 | 0.01624 |
| | | C6:0 | 4.6412549569670603E−5 | 0.00905 |
| 757 | 757 | C4:0 | 2.08045225636637E−5 | 0.01624 |
| | | C6:0 | 4.6412549569670603E−5 | 0.00905 |
| 758 | 758 | C4:0 | 2.1241646155138502E−5 | 0.01621 |
| | | C6:0 | 4.7640771250574599E−5 | 0.00903 |
| 759 | 759 | C4:0 | 2.1241646155138502E−5 | 0.01621 |
| | | C6:0 | 4.7640771250574599E−5 | 0.00903 |
| 760 | 760 | C4:0 | 2.1241646155138502E−5 | 0.01621 |
| | | C6:0 | 4.7640771250574599E−5 | 0.00903 |
| 761 | 761 | C4:0 | 3.1384437958949701E−5 | 0.0158 |
| | | C6:0 | 5.9028789795099598E−5 | 0.00888 |
| 762 | 762 | C4:0 | 2.1241646155138502E−5 | 0.01621 |
| | | C6:0 | 4.7640771250574599E−5 | 0.00903 |
| 763 | 763 | C4:0 | 2.1241646155138502E−5 | 0.01621 |
| | | C6:0 | 4.7640771250574599E−5 | 0.00903 |
| 764 | 764 | C4:0 | 2.1241646155138502E−5 | 0.01621 |
| | | C6:0 | 4.7640771250574599E−5 | 0.00903 |
| 765 | 765 | C4:0 | 2.1241646155138502E−5 | 0.01621 |
| | | C6:0 | 4.7640771250574599E−5 | 0.00903 |
| 766 | 766 | C4:0 | 2.1241646155138502E−5 | 0.01621 |
| | | C6:0 | 4.7640771250574599E−5 | 0.00903 |
| 767 | 767 | C6:0 | 1.40936982278649E−5 | 0.00972 |
| 768 | 768 | C4:0 | 2.1241646155138502E−5 | 0.01621 |
| | | C6:0 | 4.7640771250574599E−5 | 0.00903 |
| 769 | 769 | C4:0 | 2.1241646155138502E−5 | 0.01621 |
| | | C6:0 | 4.7640771250574599E−5 | 0.00903 |
| 770 | 770 | C4:0 | 8.7898627092370901E−5 | 0.01489 |
| 771 | 771 | C4:0 | 3.3066034142548501E−5 | 0.01589 |
| | | C6:0 | 7.7503698326846496E−5 | 0.00882 |
| 772 | 772 | C4:0 | 3.4654155831171199E−5 | 0.01585 |
| | | C6:0 | 9.9185236336810805E−5 | 0.00877 |
| 773 | 773 | C4:0 | 4.1810185195979403E−5 | 0.01564 |
| | | C6:0 | 7.3872568075370802E−5 | 0.00884 |
| 774 | 774 | C4:0 | 2.1241646155138502E−5 | 0.01621 |
| | | C6:0 | 4.7640771250574599E−5 | 0.00903 |
| 775 | 775 | C4:0 | 2.1241646155138502E−5 | 0.01621 |
| | | C6:0 | 4.7640771250574599E−5 | 0.00903 |
| 776 | 776 | C4:0 | 2.1241646155138502E−5 | 0.01621 |
| | | C6:0 | 4.7640771250574599E−5 | 0.00903 |
| 777 | 777 | C4:0 | 2.1241646155138502E−5 | 0.01621 |
| | | C6:0 | 4.7640771250574599E−5 | 0.00903 |
| 778 | 778 | C4:0 | 2.1241646155138502E−5 | 0.01621 |
| | | C6:0 | 4.7640771250574599E−5 | 0.00903 |
| 779 | 779 | C4:0 | 1.9144780350299398E−5 | 0.01628 |
| | | C6:0 | 3.8061485537182102E−5 | 0.00914 |
| 780 | 780 | C4:0 | 2.1241646155138502E−5 | 0.01621 |
| | | C6:0 | 4.7640771250574599E−5 | 0.00903 |
| 781 | 781 | C4:0 | 2.1021941739060599E−5 | 0.01622 |
| | | C6:0 | 4.92123990752145E−5 | 0.00904 |
| 782 | 782 | C4:0 | 2.1241646155138502E−5 | 0.01621 |
| | | C6:0 | 4.7640771250574599E−5 | 0.00903 |
| 783 | 783 | C4:0 | 3.8061763425272699E−5 | 0.0156 |
| | | C6:0 | 2.0376218965095099E−5 | 0.00939 |
| 784 | 784 | C4:0 | 2.1241646155138502E−5 | 0.01621 |
| | | C6:0 | 4.7640771250574599E−5 | 0.00903 |
| 785 | 785 | C4:0 | 2.1241646155138502E−5 | 0.01621 |
| | | C6:0 | 4.7640771250574599E−5 | 0.00903 |
| 786 | 786 | C4:0 | 4.1375982509336099E−5 | 0.01565 |
| | | C6:0 | 6.8346132976930193E−5 | 0.00885 |
| 787 | 787 | C4:0 | 2.1021941739060599E−5 | 0.01622 |
| | | C6:0 | 4.92123990752145E−5 | 0.00904 |
| 788 | 788 | C4:0 | 2.1241646155138502E−5 | 0.01621 |
| | | C6:0 | 4.7640771250574599E−5 | 0.00903 |
| 789 | 789 | C4:0 | 2.1241646155138502E−5 | 0.01621 |
| | | C6:0 | 4.7640771250574599E−5 | 0.00903 |
| 790 | 790 | C4:0 | 2.1241646155138502E−5 | 0.01621 |
| | | C6:0 | 4.7640771250574599E−5 | 0.00903 |
| 791 | 791 | C4:0 | 2.1241646155138502E−5 | 0.01621 |
| | | C6:0 | 4.7640771250574599E−5 | 0.00903 |
| 792 | 792 | C4:0 | 2.1241646155138502E−5 | 0.01621 |
| | | C6:0 | 4.7640771250574599E−5 | 0.00903 |
| 793 | 793 | C4:0 | 2.1241646155138502E−5 | 0.01621 |
| | | C6:0 | 4.7640771250574599E−5 | 0.00903 |
| 794 | 794 | C4:0 | 1.31074434096477E−5 | 0.01679 |
| | | C6:0 | 6.7280814906985498E−5 | 0.00895 |
| 795 | 795 | C4:0 | 2.0912947997407001E−5 | 0.01622 |
| | | C6:0 | 4.7392096983275499E−5 | 0.00903 |
| 796 | 796 | C4:0 | 2.46995942865043E−5 | 0.01612 |
| | | C6:0 | 5.1251123056161799E−5 | 0.00905 |
| 797 | 797 | C4:0 | 3.1221017868571102E−5 | 0.0159 |
| | | C6:0 | 8.9856519429302496E−5 | 0.00899 |
| 798 | 798 | C4:0 | 1.4541485158180799E−5 | 0.0167 |
| | | C6:0 | 5.4291331059260198E−5 | 0.00904 |
| 799 | 799 | C4:0 | 1.4541485158180799E−5 | 0.0167 |
| | | C6:0 | 5.4291331059260198E−5 | 0.00904 |
| 800 | 800 | C4:0 | 2.1241646155138502E−5 | 0.01621 |
| | | C6:0 | 4.7640771250574599E−5 | 0.00903 |
| 801 | 801 | C4:0 | 2.1241646155138502E−5 | 0.01621 |
| | | C6:0 | 4.7640771250574599E−5 | 0.00903 |
| 802 | 802 | C4:0 | 2.1241646155138502E−5 | 0.01621 |
| | | C6:0 | 4.7640771250574599E−5 | 0.00903 |
| 803 | 803 | C4:0 | 2.1241646155138502E−5 | 0.01621 |
| | | C6:0 | 4.7640771250574599E−5 | 0.00903 |
| 804 | 804 | C4:0 | 3.2720344437166301E−5 | 0.01584 |
| | | C6:0 | 3.7666353554999901E−5 | 0.00915 |
| 805 | 805 | C4:0 | 2.04826263644331E−5 | 0.01625 |
| | | C6:0 | 4.6366482807218097E−5 | 0.00905 |
| 806 | 806 | C4:0 | 3.4471807344232797E−5 | 0.01578 |
| | | C6:0 | 5.4863189909845899E−5 | 0.00895 |
| 807 | 807 | C4:0 | 3.4471292991151098E−5 | 0.01574 |
| | | C6:0 | 4.5929800536192898E−5 | 0.00902 |
| 808 | 808 | C4:0 | 3.5752706598229098E−5 | 0.0157 |
| | | C6:0 | 4.7640329357455599E−5 | 0.009 |
| 809 | 809 | C4:0 | 3.6127819240129302E−5 | 0.01581 |
| | | C6:0 | 5.1524819156835798E−5 | 0.00903 |
| 810 | 810 | C4:0 | 2.1241646155138502E−5 | 0.01621 |
| | | C6:0 | 4.7640771250574599E−5 | 0.00903 |
| 811 | 811 | C4:0 | 2.1241646155138502E−5 | 0.01621 |
| | | C6:0 | 4.7640771250574599E−5 | 0.00903 |
| 812 | 812 | C4:0 | 3.2891245107355698E−5 | 0.01583 |
| | | C6:0 | 6.5542446369305698E−5 | 0.00886 |
| 813 | 813 | C4:0 | 2.1241646155138502E−5 | 0.01621 |
| | | C6:0 | 4.7640329357455599E−5 | 0.00903 |
| 814 | 814 | C4:0 | 4.8393241944496497E−5 | 0.01552 |
| 815 | 815 | C4:0 | 2.1021941739060599E−5 | 0.01623 |
| | | C6:0 | 4.3821245403415497E−5 | 0.00908 |
| 816 | 816 | C4:0 | 4.2028903023284097E−5 | 0.01585 |
| 817 | 817 | C4:0 | 3.0379040074163501E−6 | 0.0185 |
| 818 | 818 | C4:0 | 3.5091424976887601E−6 | 0.01842 |
| | | C6:0 | 8.1248071273996598E−5 | 0.00912 |
| 819 | 819 | C4:0 | 3.5091424976887601E−6 | 0.01842 |
| | | C6:0 | 8.1248071273996598E−5 | 0.00912 |
| 820 | 820 | C4:0 | 2.1241646155138502E−5 | 0.01621 |
| | | C6:0 | 4.7640329357455599E−5 | 0.00903 |
| 821 | 821 | C4:0 | 2.1241646155138502E−5 | 0.01621 |
| | | C6:0 | 4.7640329357455599E−5 | 0.00903 |
| 822 | 822 | C4:0 | 1.6814180189790299E−5 | 0.01655 |
| | | C6:0 | 1.49213326470014E−5 | 0.00970 |
| 823 | 823 | C4:0 | 2.1241646155138502E−5 | 0.01621 |
| | | C6:0 | 4.7640329357455599E−5 | 0.00903 |
| 824 | 824 | C4:0 | 2.62922196623481E−5 | 0.01622 |
| | | C6:0 | 8.9292516060599403E−5 | 0.00881 |
| 825 | 825 | C4:0 | 8.9039211208208401E−7 | 0.01957 |
| 826 | 826 | C4:0 | 1.6382639126981499E−5 | 0.01642 |
| | | C6:0 | 4.9674118045879501E−5 | 0.00901 |
| 827 | 827 | C4:0 | 2.0912947997407001E−5 | 0.01622 |
| | | C6:0 | 4.7392096983275499E−5 | 0.00903 |
| 828 | 828 | C4:0 | 2.1241646155138502E−5 | 0.01621 |
| | | C6:0 | 6.1148459045767806E−5 | 0.00903 |
| 829 | 829 | C4:0 | 2.1241646155138502E−5 | 0.01621 |
| | | C6:0 | 6.1148459045767806E−5 | 0.00903 |
| 830 | 830 | C4:0 | 2.1241646155138502E−5 | 0.01621 |
| | | C6:0 | 6.1148459045767806E−5 | 0.00903 |
| 831 | 831 | C4:0 | 2.1241646155138502E−5 | 0.01621 |
| | | C6:0 | 6.1148459045767806E−5 | 0.00903 |
| 832 | 832 | C4:0 | 8.7898627092370901E−5 | 0.0149 |
| 833 | 833 | C4:0 | 8.7898627092370901E−5 | 0.0149 |
| 834 | 834 | C4:0 | 5.5149093214902699E−5 | 0.01537 |
| 835 | 835 | C4:0 | 7.0159993557734204E−5 | 0.0151 |
| 836 | 836 | C4:0 | 8.7898627092370901E−5 | 0.0149 |
| 837 | 837 | C4:0 | 5.8110516031289698E−5 | 0.01513 |
| 838 | 838 | C4:0 | 8.7898627092370901E−5 | 0.0149 |
| 839 | 839 | C4:0 | 8.7898627092370901E−5 | 0.0149 |
| 840 | 840 | C4:0 | 8.8360982493528294E−5 | 0.01489 |
| 841 | 841 | C4:0 | 8.2105101627525799E−5 | 0.01495 |
| 842 | 842 | C4:0 | 8.7898627092370901E−5 | 0.0149 |
| 843 | 843 | C4:0 | 8.7898627092370901E−5 | 0.0149 |
| 844 | 844 | C4:0 | 8.8360982493528294E−5 | 0.01489 |
| 845 | 845 | C4:0 | 8.8360982493528294E−5 | 0.01489 |
| 846 | 846 | C4:0 | 8.7898627092370901E−5 | 0.0149 |
| 847 | 847 | C4:0 | 9.2149492721276202E−5 | 0.01467 |
| 848 | 848 | C4:0 | 4.6171079390002102E−5 | 0.01546 |
| 849 | 849 | C4:0 | 5.7807590208202899E−5 | 0.01536 |
| 850 | 850 | C4:0 | 5.7807590208202899E−5 | 0.01536 |
| 851 | 851 | C4:0 | 5.6610693840423797E−5 | 0.01537 |
| 852 | 852 | C4:0 | 5.6610693840423797E−5 | 0.01537 |
| 853 | 853 | C4:0 | 5.6610693840423797E−5 | 0.01537 |
| 854 | 854 | C4:0 | 5.6610693840423797E−5 | 0.01537 |
| 855 | 855 | C4:0 | 5.6610693840423797E−5 | 0.01537 |
| 856 | 856 | C4:0 | 5.6610693840423797E−5 | 0.01537 |
| 857 | 857 | C4:0 | 5.6610693840423797E−5 | 0.01537 |
| 858 | 858 | C4:0 | 5.6610693840423797E−5 | 0.01537 |
| 859 | 859 | C4:0 | 6.7283419035809103E−5 | 0.01532 |
| 860 | 860 | C4:0 | 6.6932030681008002E−5 | 0.01533 |
| 861 | 861 | C4:0 | 6.7283419035809103E−5 | 0.01532 |
| 862 | 862 | C4:0 | 7.1647532994452502E−5 | 0.01523 |
| 863 | 863 | C4:0 | 6.7283419035809103E−5 | 0.01532 |
| 864 | 864 | C4:0 | 6.6932030681008002E−5 | 0.01533 |
| 865 | 865 | C4:0 | 6.7283419035809103E−5 | 0.01532 |
| 866 | 866 | C4:0 | 4.18112831510607E−5 | 0.0159 |
| 867 | 867 | C4:0 | 6.7283419035809103E−5 | 0.01532 |
| 868 | 868 | C4:0 | 6.7283419035809103E−5 | 0.01532 |
| 869 | 869 | C4:0 | 6.7283419035809103E−5 | 0.01532 |
| 870 | 870 | C4:0 | 2.1916270366483601E−5 | 0.0161 |
| 871 | 871 | C4:0 | 6.7232554716256698E−6 | 0.01723 |
| 872 | 872 | C4:0 | 9.8623264055202404E−6 | 0.01731 |
| | | C6:0 | 7.0898406466732503E−5 | 0.00903 |
| 873 | 873 | C4:0 | 6.7283419035809103E−5 | 0.01532 |
| 874 | 874 | C4:0 | 7.5107465797901495E−5 | 0.01521 |
| 875 | 875 | C4:0 | 6.8349208848892803E−5 | 0.01534 |
| 876 | 876 | C4:0 | 6.8349208848892803E−5 | 0.01534 |
| 877 | 877 | C4:0 | 6.8349208848892803E−5 | 0.01534 |
| 878 | 878 | C4:0 | 6.8349208848892803E−5 | 0.01534 |
| 879 | 879 | C4:0 | 6.8349062214906504E−5 | 0.01535 |
| 880 | 880 | C4:0 | 9.9701087779821606E−5 | 0.01495 |
| 881 | 881 | C4:0 | 6.8349208848892803E−5 | 0.01534 |
| 882 | 882 | C4:0 | 6.8349208848892803E−5 | 0.01534 |
| 883 | 883 | C4:0 | 6.8349208848892803E−5 | 0.01534 |
| 884 | 884 | C4:0 | 6.8349208848892803E−5 | 0.01534 |
| 885 | 885 | C4:0 | 6.8349208848892803E−5 | 0.01534 |
| 886 | 886 | C4:0 | 6.8349208848892803E−5 | 0.01534 |
| 887 | 887 | C12:0 | 9.9609642871672794E−6 | 0.02894 |
| 888 | 888 | C12:0 | 7.3791347134148397E−6 | 0.02914 |
| 889 | 889 | C10:0 | 2.9212843771927E−7 | 0.08302 |
| | | C12:0 | 4.0916532283562E−7 | 0.09665 |
| | | C14:0 | 6.2547862297914996E−9 | 0.1561 |
| 890 | 890 | C10:0 | 3.9921713195219098E−6 | 0.02533 |
| | | C12:0 | 1.3014523386799699E−6 | 0.03131 |
| | | C14:0 | 1.2427901953404301E−7 | 0.04814 |
| 891 | 891 | C10:0 | 1.54149843323962E−6 | 0.02629 |
| | | C12:0 | 5.0470287011740601E−7 | 0.03238 |
| | | C14:0 | 3.7693339495266303E−8 | 0.04989 |
| 892 | 892 | C14:0 | 8.2945697001932298E−7 | 0.04471 |
| 893 | 893 | C12:0 | 7.6509952186251208E−6 | 0.02902 |
| 894 | 894 | C12:0 | 9.5075588436017696E−6 | 0.0289 |
| 895 | 895 | C12:0 | 7.5723131110883999E−6 | 0.029 |
| 896 | 896 | C12:0 | 7.5723131110883999E−6 | 0.029 |
| 897 | 897 | C12:0 | 7.07995488380744E−6 | 0.02931 |
| 898 | 898 | C12:0 | 7.7707308024999804E−6 | 0.02898 |
| 899 | 899 | C10:0 | 2.8623391726682899E−7 | 0.08273 |
| | | C12:0 | 3.9884613447224798E−7 | 0.09633 |
| | | C14:0 | 5.9761084872016902E−9 | 0.1557 |
| 900 | 900 | C10:0 | 3.36786191733193E−6 | 0.08683 |
| | | C12:0 | 4.8061182424494699E−6 | 0.1007 |
| | | C14:0 | 1.1442574673726901E−7 | 0.1646 |
| 901 | 901 | C12:0 | 8.2678409466809695E−6 | 0.02913 |
| 902 | 902 | C10:0 | 1.33292595395347E−7 | 0.08454 |
| | | C12:0 | 1.95277480454429E−7 | 0.09831 |
| | | C14:0 | 3.7400036131947403E−9 | 0.1569 |
| 903 | 903 | C10:0 | 1.8523529872076101E−8 | 0.08735 |
| | | C12:0 | 3.7827293716781299E−8 | 0.1007 |
| | | C14:0 | 1.1582149669298101E−9 | 0.157 |
| | | C18:1cis-11 | 4.4636936092160943e−7 | 0.3692 |
| 904 | 904 | C10:0 | 3.2024672488398301E−7 | 0.08293 |
| | | C12:0 | 4.8431470042416999E−7 | 0.09625 |
| | | C14:0 | 5.9755054826785199E−9 | 0.1567 |
| 905 | 905 | C10:0 | 2.8916935986997599E−7 | 0.0827 |
| | | C12:0 | 4.0294123092189202E−7 | 0.09629 |
| | | C14:0 | 5.9161307729461301E−9 | 0.1557 |
| 906 | 906 | C10:0 | 2.8916935986997599E−7 | 0.0827 |
| | | C12:0 | 4.0294123092189202E−7 | 0.09629 |
| | | C14:0 | 5.9161307729461301E−9 | 0.1557 |
| 907 | 907 | C10:0 | 2.8916935986997599E−7 | 0.0827 |
| | | C12:0 | 4.0294123092189202E−7 | 0.09629 |
| | | C14:0 | 5.9161307729461301E−9 | 0.1557 |
| 908 | 908 | C10:0 | 2.9064838996105402E−7 | 0.08269 |
| | | C12:0 | 4.0294123092189202E−7 | 0.09629 |
| | | C14:0 | 5.8863686023439203E−9 | 0.1558 |
| 909 | 909 | C10:0 | 2.9064838996105402E−7 | 0.08269 |
| | | C12:0 | 4.0294123092189202E−7 | 0.09629 |
| | | C14:0 | 5.8863686023439203E−9 | 0.1558 |
| 910 | 910 | C10:0 | 2.9064838996105402E−7 | 0.08269 |
| | | C12:0 | 4.0294123092189202E−7 | 0.09629 |
| | | C14:0 | 5.8863686023439203E−9 | 0.1558 |
| 911 | 911 | C10:0 | 5.2145845868009402E−9 | 0.07313 |
| | | C12:0 | 7.4985219009258199E−9 | 0.08529 |
| | | C14:0 | 4.0745822009914199E−11 | 0.1371 |
| | | C18:1cis-10 | 6.989162215542427e−8 | 0.3182 |
| 912 | 912 | C10:0 | 1.7523345672918E−6 | 0.06245 |
| | | C12:0 | 1.6560556975478499E−6 | 0.07378 |
| | | C14:0 | 9.9754172683479696E−8 | 0.1156 |
| 913 | 913 | C10:0 | 1.05438969857545E−6 | 0.0629 |
| | | C12:0 | 1.0985192789977301E−6 | 0.07402 |
| | | C14:0 | 4.6106555954215799E−8 | 0.117 |
| 914 | 914 | C10:0 | 2.8916935986997599E−7 | 0.08272 |
| | | C12:0 | 4.0294123092189202E−7 | 0.09632 |
| | | C14:0 | 5.8863686023439203E−9 | 0.1558 |
| 915 | 915 | C10:0 | 2.8916935986997599E−7 | 0.08272 |
| | | C12:0 | 4.0294123092189202E−7 | 0.09632 |
| | | C14:0 | 5.8863686023439203E−9 | 0.1558 |
| 916 | 916 | C10:0 | 3.7224648692225599E−6 | 0.02668 |
| | | C12:0 | 1.4376674114458401E−6 | 0.0328 |
| | | C14:0 | 1.4761793085685999E−10 | 0.06159 |
| | | C18:1cis-9 | 3.3856835714823224e−11 | 0.1794 |

On BTA11, a series of highly significant markers spanning the progestogen associated endometrial protein (PAEP) gene were found. PAEP encodes the β-lactoglobulin, which is one of the major proteins in milk. The markers were found to affect C16:0 and C18:1 cis-9 in an opposite manner. On BTA13, markers with effect on de novo-synthesis of short and medium-chained acids were detected in a region that spans the nuclear receptor coactivator 6 (NCOA6) gene, which is involved in regulation of bovine milk fat synthesis. For BTA17, significant associations for C4:0 and C6:0 were detected for markers near and within acetoacetyl-CoA synthetase (AACS) which activate ketone bodies for fatty acid synthesis. BTA19 were found to contain two distinct regions with effect on fatty acid composition. The first region is close to (although not overlapping) the sterol regulatory element binding transcription factor 1 (SREBF1) gene, which is one of the major regulators of fatty acid synthesis. The second region overlaps the fatty acid synthase (FASN) gene. Within each region, extensive linkage disequilibrium among markers makes it difficult to identify the underlying causal polymorphism. Some of the markers are situated in coding sequences and cause a shift in amino acid (P #446, P #463, P #523, P #564, P #597, P #748, P #749, P #781, P #782), while some others are positioned in putative regulatory sequences immediately upstream or downstream of the gene. Such markers are traditionally regarded as more likely to be causal as compared to markers in introns or intergenic regions, since they may affect protein sequence or gene expression. However, recent research indicates that also markers in introns and intergenic regions as well as exonic markers not causing amino acids shifts may have important biological roles, and all of the markers should be regarded as putatively causal.

1.-34. (canceled)

35. A method for selecting a cattle which possesses a genotype which in a female milk-producing cattle is indicative of a desired milk fatty acid composition, the method comprising:
determining the presence of at least one allele, which in a female milk-producing cattle is indicative of a desired milk fatty acid composition, within the genome of said cattle; and
selecting said cattle when the at least one allele, which in a female milk-producing cattle is indicative of a desired milk fatty acid composition, is present;
wherein
the at least one allele is an allele of at least one polymorphism selected from the group consisting of P #242-P #246, P #311, P #312, P #314-P #334, P #339, P #340, P #343, P #344 and P #346-481 listed in table 1.

36. The method according to claim 35, wherein the polymorphisms are either spanning the progestogen associated endometrial protein (PAEP) gene on BTA11; or
in strong linkage disequilibrium with the polymorphisms spanning the PAEP gene on BTA11, wherein two polymorphisms are in strong linkage disequilibrium if r2≥0.7.

37. The method according to claim 35, wherein the polymorphisms are spanning the PAEP gene on BTA11.

38. The method according to claim 35, wherein the at least one allele is a “non-fat allele” for C:16:0 and a “fat allele” for C18:1 cis9.

39. The method according to claim 35, wherein the desired milk fatty acid composition is decreased amount of C16:0 and increased amount of C18:1 cis-9 in milk.

40. The method according to claim 35, wherein
the polymorphisms are either
spanning the progestogen associated endometrial protein (PAEP) gene on BTA11; or
in strong linkage disequilibrium with the polymorphisms spanning the PAEP gene on BTA11, wherein two polymorphisms are in strong linkage disequilibrium if r2≥0.7;
the at least one allele is a “non-fat allele” for C:16:0 and a “fat allele” for C18:1 cis9; and
the desired milk fatty acid composition is decreased amount of C16:0 and increased amount of C18:1 cis-9 in milk.

41. The method according to claim 35, wherein the cattle is homozygote with respect to the at least one allele.

42. The method according to claim 35, wherein the method comprises determining the identity of one or more nucleotides of at least one allele of at least one polymorphism, which in a female milk-producing cattle is indicative of a desired milk fatty acid composition, within the genome of said cattle, said at least one polymorphism being located within said genome at a position corresponding to position 60 of the nucleotide sequence set forth in any one of SEQ ID NOs: 242-246, 311, 312, 314-334, 339, 340, 343, 344 and 346-481; or at a position corresponding to position 60 of a nucleotide sequence which is derived from any one of SEQ ID NOs: 242-246, 311, 312, 314-334, 339, 340, 343, 344 and 346-481 by 1 to 30 nucleotide substitutions; and selecting said cattle when the one or more nucleotides of the at least one allele is one or more nucleotides corresponding to the allele which in a female milk-producing cattle is indicative of the desired milk fatty acid composition.

43. The method according to claim 35, wherein the cattle is Norwegian Red cattle.

44. A (isolated) cattle gamete, comprising within its genome at least one allele which in a female milk-producing cattle is indicative of a desired milk fatty acid composition; wherein the at least one allele is an allele of at least one polymorphism selected from the group consisting of P #242-P #246, P #311, P #312, P #314-P #334, P #339, P #340, P #343, P #344 and P #346-481 listed in table 1.

45. The (isolated) cattle gamete according to claim 44, wherein the at least one allele is a “non-fat allele” for C:16:0 and a “fat allele” for C18:1 cis9.

46. The (isolated) cattle gamete according to claim 44, wherein the (isolated) cattle gamete is a cattle semen, a cattle sperm or a cattle ovum.

47. The (isolated) cattle gamete according to claim 44, wherein the (isolated) cattle gamete is a cattle ovum.

48. A (isolated) cattle, comprising within its genome at least one allele which in a female milk-producing cattle is indicative of a desired milk fatty acid composition; wherein the at least one allele is an allele of at least one polymorphism selected from the group consisting of P #242-P #246, P #311, P #312, P #314-P #334, P #339, P #340, P #343, P #344 and P #346-481 listed in table 1.

49. The (isolated) cattle according to claim 48, wherein the (isolated) cattle is a (isolated) female milk-producing cattle.

50. The (isolated) cattle according to claim 48, wherein the (isolated) cattle is a (isolated) male cattle.

51. The (isolated) cattle according to claim 48, wherein the at least one allele is a “non-fat allele” for C:16:0 and a “fat allele” for C18:1 cis9.

52. Milk produced by the (isolated) cattle according to claim 48, wherein the cattle is a female milk-producing cattle.


SEQUENCE LISTING (916 sequences)

SEQ ID NO: 1 - DNA, 119 bp, Bos taurus
  misc_feature (60)..(60): A OR C
tctatatccccttagtcccacttaagtaaagttcaacactagccggtagcatactcagtnagtggtgggtaggatgataatgcaagagagctgaccaaagaaatacaacaacaacggca

SEQ ID NO: 2 - DNA, 119 bp, Bos taurus
  misc_feature (60)..(60): A OR C
tttaagtttctgatttacagatgtcttcccagaaaatgataatggtgtaaagaaagatanacgtcgacattctattcttggttcttatcaaatagaggaaggtctggctaagggtcagg

SEQ ID NO: 3 - DNA, 119 bp, Bos taurus
  misc_feature (60)..(60): A OR G
cacctcagaagtgacacagtccagacaggctacctgtctccttatttaaaaaaggggggnaaaagggctttctttgtttcagttttagaggcagtaaatacctttgccttggagctatc

SEQ ID NO: 4 - DNA, 119 bp, Bos taurus
  misc_feature (60)..(60): A OR T
actcaaagcaccttgatttgtaatagccaaaaactggaaacaacccaaattgtccatccncaagtgaatgcatgtacaaactgtagtatacttgaacaacagaacactatacagaagta

SEQ ID NO: 5 - DNA, 119 bp, Bos taurus
  misc_feature (60)..(60): A OR G
aatataaatcaggtattaaaagtgatgtacgagaataaaatacatgaagggatagtaagntgtttgttattttagataaataatttaagaaagacagctatccacccatgtccacatta

SEQ ID NO: 6 - DNA, 119 bp, Bos taurus
  misc_feature (60)..(60): T OR C
ccttttgggggcttggtgtgtggtttggccctgaaggcctactgagttatgtagggctgnctgtgtctacacctgtgattctcaaactgccctttaggaaagaatcaccctgtatgctt

SEQ ID NO: 7 - DNA, 119 bp, Bos taurus
  misc_feature (60)..(60): C OR A
catacaatgaaaatttataggctttagatttggatagaactgaattaaagttttgctcanaatcgcttgcttaactgtgtggctctgggtaataaataaggtggaaagtacattttttg

SEQ ID NO: 8 - DNA, 119 bp, Bos taurus
  misc_feature (60)..(60): A OR G
gcaatactttctcgttgctatgttgtattcctcctattcataataccaatgatggatatnaggattatttctagtttttagctttcaggacaaagatggctgagaaaattcctgcatac

SEQ ID NO: 9 - DNA, 119 bp, Bos taurus
  misc_feature (60)..(60): T OR A
ttatcttggtatataagctaccccttcctggaatgtcttccttccaaattcacttctttntatgtcctccccttctgaattcacatctttaaaatgccctccatttcttagatcaggct

SEQ ID NO: 10 - DNA, 119 bp, Bos taurus
  misc_feature (60)..(60): G OR A
ttttggccaatccaataaaataatcccaagaaagaatagatagataaggatatgaaagantgagcaaagtaagaagtgtacccacagagtttagttaattttactattttctttgatga

SEQ ID NO: 11 - DNA, 119 bp, Bos taurus
  misc_feature (60)..(60): G OR A
accttgagagcagtacactaagtgagataagctacacagagaaagatgaaaactctgtgntatcactaatatgtggactctaaaaatcctgaactcacagaaagagacaaaatgatggt

SEQ ID NO: 12 - DNA, 119 bp, Bos taurus
  misc_feature (60)..(60): G OR A
ttgttaaaccattagtaacgctgccatggtgggagtattgacactgtggaaattggcaantactacaaattagagtttgtttttttgttttgttttgttttgttttccccagagagctg

SEQ ID NO: 13 - DNA, 119 bp, Bos taurus
  misc_feature (60)..(60): A OR G
gcatttggcaggcaggttctttaccactgtgtcacctggaagcctaacgtttcagtaccntaacagaaatcggaaagtgtaaaaatctttaaagacaccatctttcatgattgaaagaa

SEQ ID NO: 14 - DNA, 119 bp, Bos taurus
  misc_feature (60)..(60): C OR T
taataatatgagataaagtccgagtctgagggggtattaaatgtactctcaaactggttntctagcaattacagcgttgtcaggataataaacaggggtgctattctgggtaataaagc

SEQ ID NO: 15 - DNA, 119 bp, Bos taurus
  misc_feature (60)..(60): A OR G
cctaaatcctgaccctctgctgtccagagccccgggcagctcctggtggcagtaacactngtcctctcagcctgggtcacccttgatggagggtggacgaagcagagtcctgagctgcg

SEQ ID NO: 16 - DNA, 119 bp, Bos taurus
  misc_feature (60)..(60): G OR A
tcaacacagatggggagatgtaagcctggagctggaggagcacctggctagggtctaaangcaagtcagaggtgggcggtggagcctggggtctcagcctcagaccccccaggcccctg

SEQ ID NO: 17 - DNA, 119 bp, Bos taurus
  misc_feature (60)..(60): C OR T
catcataactgtaatgctcgatcatggttgctcgctctgctagagaccgtctctcttccntgtgatcctgtgggagcattagtgagtgactaaggactgagatttcagggtcctatcca

SEQ ID NO: 18 - DNA, 119 bp, Bos taurus
  misc_feature (60)..(60): C OR A
ttatgaggaaactgaggctctgagaggcaaaaagaagttgccctggttcaaaagaaagtnaaagcttctctcccagttttgtccttatttcccacagatgtcttccatgctcatagtca

SEQ ID NO: 19 - DNA, 119 bp, Bos taurus
  misc_feature (60)..(60): T OR C
ctgactactacattaacatctccttccccatgatattgattttaggtataatgaatctcncggaatgatgagaaattcacaaagattttatcaagtctacactattcaaaatatgacaa

SEQ ID NO: 20 - DNA, 119 bp, Bos taurus
  misc_feature (60)..(60): G OR A
tatgagtgggacagacacgctagacagagttctccatacacagggaactcagtgaactcngggttatcactctactgttttgaagatagattttatactaaggagtactgtgtggggct

SEQ ID NO: 21 - DNA, 119 bp, Bos taurus
  misc_feature (60)..(60): A OR G
ttaccactagcgccacctaggaagcaccagctgtaaagtcaccatggattttaaactcanagagggtaggcacccctaaactcctcaactttcaagaggcaactgtatttgcatacaat

SEQ ID NO: 22 - DNA, 119 bp, Bos taurus
  misc_feature (60)..(60): A OR G
atccttggcaaagcacttgcttctatttcagtgagcatctatcatttttctattaagaanggaaagtaagctctcagataatgtcgtctaccttttcagtaatagtctcattgttgttt

SEQ ID NO: 23 - DNA, 119 bp, Bos taurus
  misc_feature (60)..(60): A OR C
ggaatgctaaaggcaaatcttcatagtgtccagctatccctatagggaaatccattcaanttaactttaaaaacttactctcctggggacagaaataagaactagacactatgtgatca

SEQ ID NO: 24 - DNA, 119 bp, Bos taurus
  misc_feature (60)..(60): C OR T
gctatgcttcacctttgcattttcacatgattttcagaataagcttatggacaaaaatgnatctactatgactctgagatggcattgaatttatcaatcaatttgggaaatgtttaaga

SEQ ID NO: 25 - DNA, 119 bp, Bos taurus
  misc_feature (60)..(60): C OR A
agcaccttccactccacagcacacagatgcaacacctggacatcagcagcacaactcccnatcaagaaacttgctgttaaaaaaaaaaaatccatgaagagtaatgggatggaatggct

SEQ ID NO: 26 - DNA, 119 bp, Bos taurus
  misc_feature (60)..(60): G OR C
aaagatgctcaacattgctgattatcagagaaatgcaaatcaaaactacagtgaggcacnatctcacacccgtcagaatggctaccactgaaaagtctacaaataacaaatgctggaga

SEQ ID NO: 27 - DNA, 119 bp, Bos taurus
  misc_feature (60)..(60): A OR G
tccccacctagttcatgctctcctgaaatgctcatggtcagagcagcagaaacagggatnggtggctgaactccaaatacctaaaggagcccccagccagtggagagttatctgtatgc

SEQ ID NO: 28 - DNA, 119 bp, Bos taurus
  misc_feature (60)..(60): A OR G
tatggggaatgggtccatgctgcgtcattgaacagattatttatctttttgaaaaggtcntgtatgtttagcgtgaagattcatcaggagcagaaaatataaaatgaaaagtaagcttc

SEQ ID NO: 29 - DNA, 119 bp, Bos taurus
  misc_feature (60)..(60): A OR G
tcccgaagagacgccgctgtgcttgttagctttaagaagcacacagctttattgacaaangcatcacctccataactaagaaaaggggtgatcgacaatttcatatttccactttcagg

SEQ ID NO: 30 - DNA, 119 bp, Bos taurus
  misc_feature (60)..(60): G OR A
aaaacacagtcttgcattttaaaactcggcaataaatacgagaaatccattcattgaganccatcttcattactgaaatggaaatagattcactgagacattattgactatctcacgag

SEQ ID NO: 31 - DNA, 119 bp, Bos taurus
  misc_feature (60)..(60): G OR T
aggggtaggtgagggagaagaaggtgccaggctgatcacctagggctggtgacgagtttngggttttattatctgtgctagaggctagtaatgaggtttatgtgaagtgatgtgatggg

SEQ ID NO: 32 - DNA, 119 bp, Bos taurus
  misc_feature (60)..(60): C OR A
tttattgacacagggccacacacacctgttgaggaattaccatggcagcttttctgttanaacagcagagttgaatagtcatgacaaaccgcaaagcttgacatattcaccatctggtc

SEQ ID NO: 33 - DNA, 119 bp, Bos taurus
  misc_feature (60)..(60): G OR A
agcagctaagcacagcacagggttgcctgtagttttccagtagagcatgggtttctgaanccatcgttagaagaggccccaagttgctatctgagcactgggttcagccatcacttctg

SEQ ID NO: 34 - DNA, 119 bp, Bos taurus
  misc_feature (60)..(60): A OR G
ggcattttgtctgtctgtagttgttaactattacaggcaatgctgtaagtgttatctgtntagtcttagaagtggcaaaagcctccttaggtcatcagtattgtcctccttgccctctt

SEQ ID NO: 35 - DNA, 119 bp, Bos taurus
  misc_feature (60)..(60): A OR G
acctgttagcttcctgccctttttaaatgagttatctaatctccttataccgtaccttanttatttgtaagtaaataatggtgttggcttcccagcccggtggtaaggaatcagcccgc

SEQ ID NO: 36 - DNA, 119 bp, Bos taurus
  misc_feature (60)..(60): A OR C
tttaaaaaaatagcttttgaaaagatttcaggtaatttaattggtatttctatttacttnctcagtttggtcttaaatactttatattggtgtttaaagttctccattattaggaagaa

SEQ ID NO: 37 - DNA, 119 bp, Bos taurus
  misc_feature (60)..(60): C OR A
tgtgttttctggaatattagggaatatggtagtgaagagttctaaccttgaggctatttntccaagttaaaatggtcttgaagattcccattaagctcgaaggttgggaactgggtggg

SEQ ID NO: 38 - DNA, 119 bp, Bos taurus
  misc_feature (60)..(60): T OR A
ttaggaaaggggcaaaaatgagccactttacagtggagaaattcaaataatactaccttntccatgttctcaatatcaccatcaacagtgatgttggtagtgtgtatccttgatataat

SEQ ID NO: 39 - DNA, 119 bp, Bos taurus
  misc_feature (60)..(60): C OR G
gagggagctgtcgattggacattttccaccaagaaaagcagttgctcttcagtttcaaanatagttacacctagttttgatatttataataccagcaagacaaagcgttctaaacagat

SEQ ID NO: 40 - DNA, 119 bp, Bos taurus
  misc_feature (60)..(60): C OR T
aagtgtgggaagcaggcacagactcccgtgcagagagaaggtgccaagaatgaggccatngtggaggaaatgatgcacaggtgactggggaagaaaagggaacaggaaaggtgagagga

SEQ ID NO: 41 - DNA, 119 bp, Bos taurus
  misc_feature (60)..(60): G OR T
tttctgtcctctcttcagagatgacatatttttattctaggctcaaagaagtggggaagngtaaagggtaaaataggctaacaaattcttgctgctgctgctgctgctgctaagtcact

SEQ ID NO: 42 - DNA, 119 bp, Bos taurus
  misc_feature (60)..(60): T OR C
tttgctaaaatatgagcgaacaagtccagtgtagggagttgaacagagcactatggtatngagtgtgtagtttcttgccttacgatgcctactatgattttaaagttacttaaaattgt

SEQ ID NO: 43 - DNA, 119 bp, Bos taurus
  misc_feature (60)..(60): T OR C
tgtgatcagtcagcagacagaggacagagctgctgaggtcttgggggcccaatgggtacntgggtgcttgtaacattcctgaggatagtagggactcttttgataacagcctttctctg

SEQ ID NO: 44 - DNA, 119 bp, Bos taurus
  misc_feature (60)..(60): T OR G
gtaaacagtctaactgcatcccagatgcagaactctcatattccggttattacaggctgncagaggagcccctgattcctctcccagcatcctagaaatgtacctgatgtcctctggtg

SEQ ID NO: 45 - DNA, 119 bp, Bos taurus
  misc_feature (60)..(60): T OR C
atagaaccaatatttctcagctcaggatatcattggtataaaatctccaattgcctgaangctgattcttcctcgtgcttcaaaaagctcgtctctgcttctctcctttccccaaggtg

SEQ ID NO: 46 - DNA, 119 bp, Bos taurus
  misc_feature (60)..(60): C OR T
aatgaatacccacaatctttcctgaataagccatcttcttcccaactgattagaaacaangcttttataagtactcatttttattttgtataatgatcaaatgtaccatctattctcat

SEQ ID NO: 47 - DNA, 119 bp, Bos taurus
  misc_feature (60)..(60): C OR T
ccaccccccaggtgcgcacccggcttcttcgggaaccccctggtgctgggcagctcgtgncagccctgtgactgtagtgggaacggtgaccccaacatgctcttcagcgactgcgaccc

SEQ ID NO: 48 - DNA, 119 bp, Bos taurus
  misc_feature (60)..(60): G OR A
gaagtggaccctccaggcttaggcaggaggattcaggaggggttgtctgctcagggaatngggtggttgttgggaggtgcagaggaaggaaaccaatgtttttctccctttgcccccat

SEQ ID NO: 49 - DNA, 119 bp, Bos taurus
  misc_feature (60)..(60): A OR G
agggctacagtccatggggtcgcaaagagttggacacgaccgagccactaacattgtcantcaccagtcaggatgcacagcacttctggtccatctctgttcccctgcttgctccccca

SEQ ID NO: 50 - DNA, 119 bp, Bos taurus
  misc_feature (60)..(60): A OR C
tgctgcgacttgcttagcttttcctcctctgtgcccttgatattgctgcctgtggtcacngtgattttgctggggtcctgcacacaaaccaaagaaagggagacacttgagggcaggag

SEQ ID NO: 51 - DNA, 119 bp, Bos taurus
  misc_feature (60)..(60): G OR T
gcatggcagagcagaacagtagcagtgcttcctcagccactcggcaacaaatgcctcctncaccccaaaggaagactgacgaaaagttctaaaattagaagatgagctggggctgaaag

SEQ ID NO: 52 - DNA, 119 bp, Bos taurus
  misc_feature (60)..(60): G OR T
tttctaaataatttaaaagaaaaaggagactaactccagcaatgaaaaggacaggcaaangacatcaaaatagagggagaatgctaattaataccagtgtttaggaaggcaactgggca

SEQ ID NO: 53 - DNA, 119 bp, Bos taurus
  misc_feature (60)..(60): A OR C
ttagtccctcaaaatttagttcatcatgcattatctcagtgaagcccttccttctatatnctgtcctaactcaacccttcagaataatcaccctctgtgagcttcaacagacctgagac

SEQ ID NO: 54 - DNA, 119 bp, Bos taurus
  misc_feature (60)..(60): G OR A
ggcaacacggtatggacgttcggggtgagacacagacctgtatgctataccaaggggccnaggctgctgcttcctgctcagaacataacccaaggctgttcagtccaatgggccctctg

SEQ ID NO: 55 - DNA, 119 bp, Bos taurus
  misc_feature (60)..(60): G OR A
ttgtgtgaccacaggcacatccttctcttacagtacaatcgccccttttacacaaggaanagaatggaaaggaaattttcaatcagcaaactctaaaggagacacaatttgggactcta

SEQ ID NO: 56 - DNA, 119 bp, Bos taurus
  misc_feature (60)..(60): C OR T
aggtggctccaggctgttcacagtctcctgggagtggttacctgcaccagtagggtctgngaagtgagaacagaggacagtccttcttcctccaaatctaaaccctgaaagccttttgc

SEQ ID NO: 57 - DNA, 119 bp, Bos taurus
  misc_feature (60)..(60): G OR A
tggggccactccaggtaccagtatggaaaggtgagggggggtaggtggtgtgccagcacnatgctgcgagggtggtcctccttctggaacactgttcaacatccggaggatgcaatgga

SEQ ID NO: 58 - DNA, 119 bp, Bos taurus
  misc_feature (60)..(60): C OR T
gacagcaacctcctgggagactctgagccagaagcccccacccataccagctgcagagtnctagaaaaaagtggatctcctgacaaaaagtgaccagatccgctgagtacagaggaaga

SEQ ID NO: 59 - DNA, 119 bp, Bos taurus
  misc_feature (60)..(60): A OR C
cctcagggaagccttcccctttaaagccacacagtcggtcactcaggcttcctgtccgcngtggcctgtgtttgcattagtagcgctcttatcccaggatctcataagtagctgtttaa

SEQ ID NO: 60 - DNA, 119 bp, Bos taurus
  misc_feature (60)..(60): C OR T
ccgggagtcactcaaaagtaatgcccagaactccagcccaggctgtgcctctgagtaagngagggccctcctgggcctcagtggccttacctgagaaatgggacttgattgtgtgtttg

SEQ ID NO: 61 - DNA, 119 bp, Bos taurus
  misc_feature (60)..(60): A OR G
tgggggcgagggacgctgactcccagtcccggattactaggtctaaagttgagttgactncacttctgattctgatctcaaggcgtaatctgattctacagagttgatattctacatgg

SEQ ID NO: 62 - DNA, 119 bp, Bos taurus
  misc_feature (60)..(60): G OR A
catttattcccagaatccagagggctgaaccaacaagcacccctcttcaccccatttgangaagggaccaatctagcctccagctggagacatcacaggtccagggtttgtgaggctgc

SEQ ID NO: 63 - DNA, 119 bp, Bos taurus
  misc_feature (60)..(60): A OR G
gagtcccccttaccccatggacttggatctcaccaagggcaggactgagatgtcaggagnaaaagaaaactcatcttcttaatacagttattggtataatctgaaaatatgactaataa

SEQ ID NO: 64 - DNA, 119 bp, Bos taurus
  misc_feature (60)..(60): C OR T
gcaggctgtcaccacctgtgcctactgtgccagtgggctcgcagcaggggcacaataaangaattgcccctcctcactgtgtgtccctgttcctcccatgaaactgtcagggcaccgct

SEQ ID NO: 65 - DNA, 119 bp, Bos taurus
  misc_feature (60)..(60): C OR T
atctcaggcactgtttgttcacaccattccttctgcctggagtgaccttcctgtacacangcccttcaaatcctgcttcccttcaaaccccgtttgccacaggcttggtgaatggctct

SEQ ID NO: 66 - DNA, 119 bp, Bos taurus
  misc_feature (60)..(60): A OR G
gcaaggtgaggagggatcactcagaacctttcccattgccacgcagagggcataggcccnagaggtaagtaaccaggcttcagatcagagccagaccaggcagaaactccaggcccttg

SEQ ID NO: 67 - DNA, 119 bp, Bos taurus
  misc_feature (60)..(60): A OR G
cccagccttagtcagttcccaagccccctcagcagcaagcctgcgggctggagatcttgnggttttctccctggtctgttgctctgactgggcttccactgttcagctggagggttcca

SEQ ID NO: 68 - DNA, 119 bp, Bos taurus
  misc_feature (60)..(60): C OR T
gcaagtctgtgctacaaggctgcctatctgggcttgggacaactacctcctcctcctgcnaggttctagctgagcctggcctctccccatctttatacagggaccagttctgttccaaa

SEQ ID NO: 69 - DNA, 119 bp, Bos taurus
  misc_feature (60)..(60): G OR A
tgtctgtaccatcagcccggcagttgggcagcagagaactcagctgcagcggccagcacngtggctctctgccctttctcccccgagacagccttaagtccttccaagagtggggagcg

SEQ ID NO: 70 - DNA, 119 bp, Bos taurus
  misc_feature (60)..(60): T OR G
cattgaaactttcattaaaaaataaaggagtgtgtacaatccattacttctggggccccncttcctagaccccacgtgactcctggctctggcttcagacaccctgcaggctgcagcct

SEQ ID NO: 71 - DNA, 119 bp, Bos taurus
  misc_feature (60)..(60): A OR G
ttccagggaactttttgccgaaatgtcggggtgcccggctgcaggcacaacataagatcnatttcattctcaccccaggcactcagggaggcagaaagtccacaggctacttggacaca

SEQ ID NO: 72 - DNA, 119 bp, Bos taurus
  misc_feature (60)..(60): A OR C
ccctctgctccgtgggcctggaagctctcctccctggaatctggggccagaggggagaangcccctggcctgacatccaaggccgcacagtcagaccctatctatcttccaaatctctg

SEQ ID NO: 73 - DNA, 119 bp, Bos taurus
  misc_feature (60)..(60): C OR T
ttcagggagtcaaacagacttgaggttgaatcctgactgccatttgctgtggcattcttngtgcccacagatcccagttgactgcttcccacctgaattagcccatcagttgacccttt

SEQ ID NO: 74 - DNA, 119 bp, Bos taurus
  misc_feature (60)..(60): G OR A
gagccttctcataggacgcagaagaagctgacactggatctgtcccgtcacctgaactcntcggtaatttattgatgctggatgctggcacctagcagcttgccgggtcagcctcaggg

SEQ ID NO: 75 - DNA, 119 bp, Bos taurus
  misc_feature (60)..(60): T OR C
gaggaatctgaaacctgaggccatgagacagcacatctgtctcacctggctgtgtttccntggcatttgcctgccctgacatgctgggaccctaggatgtgtgaggatccagctaaagt

SEQ ID NO: 76 - DNA, 119 bp, Bos taurus
  misc_feature (60)..(60): A OR G
aataatccatttttttatagaaattcattgtaaaaccaattttttggctactggggtggngaatttgtaactggatcccgccttctaggcggggtcacatctgcccgaggtgggatctc

SEQ ID NO: 77 - DNA, 119 bp, Bos taurus
  misc_feature (60)..(60): C OR T
gacaaaggcagagatttgtatctggaatgagtcagcaactgagtagggtccagaagtggnaagagggaaaggaacttatgaaggtgacattgaccggttagggtgaggccagtcaaggg

SEQ ID NO: 78 - DNA, 119 bp, Bos taurus
  misc_feature (60)..(60): A OR G
tatttggagagggactctgggctggcagccacatttgggacccccagagggtctgaggantctgaggcccagggaggggctgggacccgccaagctctccccgtgagtcaggtggctgc

SEQ ID NO: 79 - DNA, 119 bp, Bos taurus
  misc_feature (60)..(60): C OR T
ggtcaccatgaagacaggcaagagcctgctgcacctccacggcaccctggagatgtttgntgcacggtggcggggcaagccgcctgtatccctctttgtcctggaaactgtgagtggac

SEQ ID NO: 80 - DNA, 119 bp, Bos taurus
  misc_feature (60)..(60): A OR G
tgcggccctggcaatcatagaggtgagtttcccatggtggggagggtaagccttgatggncctaaagcttgtctctggagaggggactgctctgcctttgacaaggtctgtcgctgccc

SEQ ID NO: 81 - DNA, 119 bp, Bos taurus
  misc_feature (60)..(60): A OR G
gactggatacctgcctgggtgacccttttctgaacctcagtgatcttgtctgcagcatgngcatagtcaggagacctctcagggatcaacagaagggcctgagtcccttgaatgacatg

SEQ ID NO: 82 - DNA, 119 bp, Bos taurus
  misc_feature (60)..(60): T OR C
ctgaatgagacctttgcttctccgcagccatctcgggcaccttgcagcaaagtgatgctnttcgctcagccctgagagaggtgcccatgggtaaagctggtggtgatggtggcgggcct

SEQ ID NO: 83 - DNA, 119 bp, Bos taurus
  misc_feature (60)..(60): G OR T
tccattcctggaggcacacaggtgacccagaaggcgagagggacccagggggctgggacncgtccctgggggtctgaaaagggccctaaggaatgcggagggacagattggtgagagga

SEQ ID NO: 84 - DNA, 119 bp, Bos taurus
  misc_feature (60)..(60): T OR C
gggggtctcacagggagcccccagcgagcaatgaccgctgtatgatggcagtgacattcnattcaccttccctttcctccaggacacagaactcttggccatgttttccgtggagaatg

SEQ ID NO: 85 - DNA, 119 bp, Bos taurus
  misc_feature (60)..(60): G OR A
ttggttcctccttcgtggaattcctaaaggatcttgccaggtagtgatgatgctcatgcnggcatagcacccaccgggtaccaggggtgattctatgggatttacttgaattaccctat

SEQ ID NO: 86 - DNA, 119 bp, Bos taurus
  misc_feature (60)..(60): C OR T
gacttcctctccagccacagggctggggttcggggtgggggctcaaagatcccttcaganctgagcgaatctctgccttttcattcttcctctgcaggttggcctcttggaggtgctgg

SEQ ID NO: 87 - DNA, 119 bp, Bos taurus
  misc_feature (60)..(60): A OR G
ggtaaggggagattagagctctgttctgccttcctctgcaaacccaggaattgggaggangaagagaagggaagggaagggcaggacaatagcagcctctaccttcaaaggatggggct

SEQ ID NO: 88 - DNA, 119 bp, Bos taurus
  misc_feature (60)..(60): T OR C
cttgaatgggcaatgtgggtcagacgccggctctgggccatccttgggggcaccagtcgnggtcccttctcttgagcgcctgcaggacctcagcactactctgagcctcagtttcctca

SEQ ID NO: 89 - DNA, 119 bp, Bos taurus
  misc_feature (60)..(60): G OR T
ctccactgttcagagttactgctgccgagaaatgcgactggcctgaccctaaggcaggcngctgtctgctgagctgtagccagaagccaccccattcactaaccagctctgtgttctgc

SEQ ID NO: 90 - DNA, 119 bp, Bos taurus
  misc_feature (60)..(60): T OR G
cattcctcaattctcccaccccaactccacagcgcacggggagggtcagcactcagaagnagcaagttcaaagtcattagactgactgcacactgctggagggctgccctctggcctaa

SEQ ID NO: 91 - DNA, 119 bp, Bos taurus
  misc_feature (60)..(60): C OR T
ctggccatgtacggtctgctcagagcaactgctccccaagccccatcccaaagctccttntctgccagacagtgaggaaaacggccaatctcttcatgttgaaaacaatcagacagcat

SEQ ID NO: 92 - DNA, 119 bp, Bos taurus
  misc_feature (60)..(60): A OR G
gagtccttgccaggagagaagaggagtctttcttcttcctattgtcatgggatgtgaganttccccattctggcctcccaactctactgaagtttctgtttaatttgtgcacctctgat

SEQ ID NO: 93 - DNA, 119 bp, Bos taurus
  misc_feature (60)..(60): T OR C
gactttgaggaatattgctttcgtttaccatccccattgggcagtcagggacactgaggngggaaagagcaagcaggtgccagggttaggcttggactcggttgggcttgacctctttg

SEQ ID NO: 94 - DNA, 119 bp, Bos taurus
  misc_feature (60)..(60): C OR A
gggggtcaggggcctctgacttgccttgcctctgattcctctccactgggcatgggcccngggagacccccagtgaagaggagcgaacagtaacagagacatccatcctcgtggtcaca

SEQ ID NO: 95 - DNA, 119 bp, Bos taurus
  misc_feature (60)..(60): T OR C
ttggagtaaggagcaaattcccagggggcgaaggttgccttggggaatcaggtgaaagcngtggtttcttcccagaacttagtggatgtgcccttggagacatgagcacaccagagagc

SEQ ID NO: 96 - DNA, 119 bp, Bos taurus
  misc_feature (60)..(60): A OR G
ggatgccgaggggaggggctgggtgaggattgttacactgagtgtctgcaaacagcaacnatagtcctagctgtcttattccatttgttctacttatgaggaataattcctgcctcctt

SEQ ID NO: 97 - DNA, 119 bp, Bos taurus
  misc_feature (60)..(60): A OR G
tctgggtttctcaccaagactgggtctcaggcaggaggcaggaaggagatgggacccccntcaagcaggaggggcaggctgttgtccccctggggagccaaggagggcctgactcatcc

SEQ ID NO: 98 - DNA, 119 bp, Bos taurus
  misc_feature (60)..(60): A OR G
ttccctcagggaagtcacccatgggcagggtccctggtctatgtgctataataacagcanatgcaacatgtcaggagacacgttcatcagcgacgatcacatcgttatgacacagggga

SEQ ID NO: 99 - DNA, 119 bp, Bos taurus
  misc_feature (60)..(60): T OR G
gcctcccacccatcaccacaatataagggccatcagcacccagaattccaggagactgcncagtggagcaaggattttgtaagtattctcctccaaacctgtgaatggtgggcttcccc

SEQ ID NO: 100 - DNA, 119 bp, Bos taurus
  misc_feature (60)..(60): C OR T
aggtggtttgcagggtgcttccagtggataatgatcagtcctctttctctcgtgtctctnctctccccgatctccatcccaacatcattcctgtcttcctctctccatctcccgctgtg

SEQ ID NO: 101 - DNA, 119 bp, Bos taurus
  misc_feature (60)..(60): G OR A
atgaaggctgcctcacaaggtcaactcacgtcagggaacccttgagcccctggggttatntccggggccttcaaccagctctcctcagctctgccgagcggaccttaggtcaggctgtg

SEQ ID NO: 102 - DNA, 119 bp, Bos taurus
  misc_feature (60)..(60): T OR C
tgcttcttaagctgtgtaccacactaaatggagactgttttgcagctatctttcagcaantgaagactgatttcatgctcccccaggaatccaatacttctgaggagggccaggagaag

SEQ ID NO: 103 - DNA, 119 bp, Bos taurus
  misc_feature (60)..(60): T OR C
cagacttcagtacaccaagcttccctgtccttgttcagagagatcccatataatgtccanatagaagagagactaaagtgggcaatgtgaagggacttctccatctccattcactgtga

SEQ ID NO: 104 - DNA, 119 bp, Bos taurus
  misc_feature (60)..(60): G OR A
gttctggctgagagagaactagtctggagttgaaggctggggcaagttggccttagcagntaacaaggctgagtcatgagcagaggtcagttcatcctgcccaacaggtgtctgatcac

SEQ ID NO: 105 - DNA, 119 bp, Bos taurus
  misc_feature (60)..(60): A OR G
aaagcctggatgcagattatcttaagaaccacatcactaagtcacagcttggggaaatgntgagggggcatcctatgcatccttacaggggagggcatgctgctctatgccagctgcac

SEQ ID NO: 106 - DNA, 119 bp, Bos taurus
  misc_feature (60)..(60): G OR A
caggagtgacaactgagcccagtcaaaggacactctcagatactcctccctacaatcagnacacactatgctcatcatcctccatccccagcaataaagagccatttctgcatcctgtg

SEQ ID NO: 107 - DNA, 119 bp, Bos taurus
  misc_feature (60)..(60): C OR A
cagccacctctccccagcccccagcaggtcccgctgtcctggccttgatctctcccaccnaggactgatgagttttctctcggggcccttctcaaagtgcaagtcctcagtttcccctt

SEQ ID NO: 108 - DNA, 119 bp, Bos taurus
  misc_feature (60)..(60): T OR C
accctgctaccttagtctcccaatctgtaaaataaggaggcctttgcagctctagaatcntcacctttaaaatgttctaagaaacttttcccataatcaagtctctctttaaaaagaat

SEQ ID NO: 109 - DNA, 119 bp, Bos taurus
  misc_feature (60)..(60): A OR G
tccaaaactagcaacacatttgtagaccatgtctccctcctccaatctgatgcctactcnagggtcacacaaccacactgggaacaacaaatatcaaagccacaggagagcttagaggt

SEQ ID NO: 110 - DNA, 119 bp, Bos taurus
  misc_feature (60)..(60): A OR G
ctggggacccaaatgctgggccctttccttgtgagatcttggccagtcacctcccttccntaaacctctttcctcagcagaaaatggggctttctgaacctcctcacagagttgtcctg

SEQ ID NO: 111 - DNA, 119 bp, Bos taurus
  misc_feature (60)..(60): G OR A
cctctccctcctcacctcctggcttctttctttccttccctctttccaattctctctgcntgtgtctctctctcttttgctggctctgtatgaccctcagctgtgcccacgattccgcg

SEQ ID NO: 112 - DNA, 119 bp, Bos taurus
  misc_feature (60)..(60): A OR G
ttaggctggctctgagaacaagcccagtggctgggtctgcagttggctgtgggtttcacnagacagctgtatggagctgttaggggtgcttggggtcagcttgtagcccaggactccta

SEQ ID NO: 113 - DNA, 119 bp, Bos taurus
  misc_feature (60)..(60): T OR C
gtcccatcagtgacctcctggcaaccctcacctgctccctctctttccatcctcacaccnactggccgggctcatagtccactttctgaacctcctccggctttccctgactcagacct

SEQ ID NO: 114 - DNA, 119 bp, Bos taurus
  misc_feature (60)..(60): A OR G
gggaactcactgtgaatggaagacacaagaagtgattagatatcgtttgaagagtttcantgcaaatgttcccagcactgggccttggtcatcatgaaaggatgagagaccccttcagt

SEQ ID NO: 115 - DNA, 119 bp, Bos taurus
  misc_feature (60)..(60): A OR C
tgctggattccaagggagtgaagaatggtgatagagtggtaaagtagaaacattgccccnaaaagatctgcattcaagttcaattcaacccaataagaagttccatgagcatatggaca

SEQ ID NO: 116 - DNA, 119 bp, Bos taurus
  misc_feature (60)..(60): A OR G
ctggaaggtcatagtccagacagatgggctaaggtcctgcccaaggcccaaggaatcgcnacacagaacacttttgggtgtccagcaaaagagtaaaaagtaaaggctaaatttaaagg

SEQ ID NO: 117 - DNA, 119 bp, Bos taurus
  misc_feature (60)..(60): G OR A
gatcagagcaggcattattagtttgccaggtgagggaattctacctcgtaatataaatantgtcttgaacaggactataacttttttcttttaaaatatatttcctaaaagccaaccag

SEQ ID NO: 118 - DNA, 119 bp, Bos taurus
  misc_feature (60)..(60): C OR T
gaaggtggatttttaaccactggactgccagggaagtccctcctttctgccattctgcanctattgttgcctccttccaaaccgggttgatgctgtaaagcccaaagctgaccatgtga

SEQ ID NO: 119 - DNA, 119 bp, Bos taurus
  misc_feature (60)..(60): T OR C
aaaaaaagaaagctaagacgcaatcaagaatgagattcctaggccccatataaatatcanttcttccagggagcactcaggtattgaagttactggcttcctccattgtccaccccaac

SEQ ID NO: 120 - DNA, 119 bp, Bos taurus
  misc_feature (60)..(60): T OR C
gaggccaagagaggtggtggcattgatttgacccagtggtcagactctagatgaggcccngacagggaattctgtctttatttcagttgctcagtcctttctatgatagagaatgctgt

SEQ ID NO: 121 - DNA, 119 bp, Bos taurus
  misc_feature (60)..(60): A OR G
ggcgctttctccccgtctggtgtctcgtgcagtgcacagctggggctcataggtagccanaacagctttgggttttttagagaccttgtttataaatccttcatttccttctttgagcc

SEQ ID NO: 122 - DNA, 119 bp, Bos taurus
  misc_feature (60)..(60): C OR T
gacaacgtgggtatcaggctttagagctggaatgtggaggatccagactgccaggctaangcatctctgcacaggacattgggcatttgaatttcttatgtaaatatctcctggtcaca

SEQ ID NO: 123 - DNA, 119 bp, Bos taurus
  misc_feature (60)..(60): T OR C
tagtatagccccctaccctgccacctaagggtacttctgtccttggactcctggcccatntggactttcttcaggcccagattgacagaagttctaacaataaagttcagaagaatgag

SEQ ID NO: 124 - DNA, 119 bp, Bos taurus
  misc_feature (60)..(60): C OR T
atttgaaacaggattccattccaagataaaagttgtgattattaacttatatggtacttnttcttgtgtaacttttaaattgcctttagaattctaaagatattttaagcaacttgcag

SEQ ID NO: 125 - DNA, 119 bp, Bos taurus
  misc_feature (60)..(60): A OR G
tcagtatttggtacagctagtttggctgagaaactaaaccagaacaaaaattaagtgttncttcttccagcttttggcatgtcagaaactgtggctatgagccagaagagtaacaggct

SEQ ID NO: 126 - DNA, 119 bp, Bos taurus
  misc_feature (60)..(60): A OR G
ttaagggtacacaagtagcctttacaaaatccttctttgtgcgactacttaacctccccnaagatcaagaagcaggaatcagaagttatggattcattgaatgttaaaataggtctttc

SEQ ID NO: 127 - DNA, 119 bp, Bos taurus
  misc_feature (60)..(60): C OR T
tgagatcgtgctgttttgtagctcaaaggggaaaaacaggcaggagatggagatgcgtgngaatggatcagagattacaaatccacccctgaagacagaccacattttgcttggtagga

SEQ ID NO: 128 - DNA, 119 bp, Bos taurus
  misc_feature (60)..(60): G OR A
atgggcattgaagcttgagaaccacagcactagatgatatgaatggggtagggttatagnaatctgtatctttagcatgctttcctaaggtatattgcaggaaacactgacaacggagg

SEQ ID NO: 129 - DNA, 119 bp, Bos taurus
  misc_feature (60)..(60): A OR G
ataaggatcttcattactctcagccgtggcactctctttaatggtggatcatgtatatantctcataatttcccagctttaaaagaatagaaaaggtaaaatcaaagtagttcaattgc

SEQ ID NO: 130 - DNA, 119 bp, Bos taurus
  misc_feature (60)..(60): G OR A
tatttacacatttacaccttaaactttaaaatcatgccagttcattagtcacactttctntgatttgactcttaatttatagaaataacatgaatagccattttgctgttccagtttga

SEQ ID NO: 131 - DNA, 119 bp, Bos taurus
  misc_feature (60)..(60): T OR C
cccagaagaatttaaacttcatttttcaggcgttagggaatcattgaaaagtattgacanggtcacatctgttcacaattctagtagctatgaggaattgggctggagaagtgagagta

SEQ ID NO: 132 - DNA, 119 bp, Bos taurus
  misc_feature (60)..(60): C OR T
gcatattataaccttagaatgtattagcatctgggttctcaggattctttatcttgttcntaaacatctaaacttacatatactaatagatcaggtaaatatgtatggtgagataaaca

SEQ ID NO: 133 - DNA, 119 bp, Bos taurus
  misc_feature (60)..(60): C OR T
ttggggcatatgctttatttgtggaaccagggatggctctgatattaaatgagaagcacntgatagaccctcatcattgatccaggaaataaaatcccagtttatagcctatgttgatg

SEQ ID NO: 134 - DNA, 119 bp, Bos taurus
  misc_feature (60)..(60): A OR G
ataacaaagaagcagttaagttacatacccccattatattttcagaagtgagatggtacnatatgaaaaaaaaaatctctgaatggagatgtcagtttccatcctgcatcagctagagt

SEQ ID NO: 135 - DNA, 119 bp, Bos taurus
  misc_feature (60)..(60): G OR A
aaggcctaccacccacggtgataggtgcattttctggtgcatctgaccctcgcagttcanctaagatatgaaatgatctttttttccttttttgtttagtattctaaacgcatatgacc

SEQ ID NO: 136 - DNA, 119 bp, Bos taurus
  misc_feature (60)..(60): A OR G
atcactcccgtgcaggcatgatctcagactcgagtaactgccctgcaggacagtggcacngtgctgggactatagaacgggggcgctgaccacgtccaggggtggagggagacgtgagg

SEQ ID NO: 137 - DNA, 119 bp, Bos taurus
  misc_feature (60)..(60): G OR A
tggttgcaccaggtaaggggtcttccccacccctacttgagagcagaccctcactccatncttggtagctatcgggaaggcttcgggtgtgggtgtgtccatgggttccacagaaaggc

SEQ ID NO: 138 - DNA, 119 bp, Bos taurus
  misc_feature (60)..(60): G OR A
ctttcaagctcctttgtagtgtcttgctccaagtgtctcatgtgtcaatcccccaccccnaccacctttatctttctctaaccgagaacgccacaaagccaaggccgtgtgttttagtg

SEQ ID NO: 139 - DNA, 119 bp, Bos taurus
  misc_feature (60)..(60): T OR C
agggagcatcatgtgcgaatctcccgccaaatcccactccagccagcatgcacttccctnatcctgacccttcctcagatcacgcctctcagcgcaaaacccgtgtgggacacaccccc

SEQ ID NO: 140 - DNA, 119 bp, Bos taurus
  misc_feature (60)..(60): G OR A
ttgcacccccagagctgccagctagaaaggattgacctggactagtgctggtgattacanagcgtgtgctcccctgtctctgctcacagagaagcaccggagacccagcaggagagcct

SEQ ID NO: 141 - DNA, 119 bp, Bos taurus
  misc_feature (60)..(60): T OR C
ccactgttgttagggaagctcccaggtacaggcggattggccaaaggagtaggactgacnaatacatttcttggcaactctacattttgcaacaaggttgcatttgtttgagaggcaag

SEQ ID NO: 142 - DNA, 119 bp, Bos taurus
  misc_feature (60)..(60): G OR A
gcagggcccgggcgggcctgagggacgtcagctaggagagaagtgggggttcacaggagngggaggaattgggggtgttccgtcgggaaatggaagttccgtgaggagatggaggttcc

SEQ ID NO: 143 - DNA, 119 bp, Bos taurus
  misc_feature (60)..(60): C OR A
gatattgctgcagaatccttgaggcccttctttctagtttccctaccatctgtacttgantagagcctgtctttccacatatgtaaaatgaaaggagtagattagatcagtgttttccc

SEQ ID NO: 144 - DNA, 119 bp, Bos taurus
  misc_feature (60)..(60): C OR T
ctgtgctttgcccttgcgtgtacctagtgccttttctgtgaccatccacaccagcaccanagactagctgcatttgctcaaagcactattgacacacttcttcttaaacacattagtgc

SEQ ID NO: 145 - DNA, 119 bp, Bos taurus
  misc_feature (60)..(60): T OR C
gttgttggggaggggctgtagtgagctagtttctcaaaccacaaacagatcccaggggcngactgccctcttcccactcagtctccatcatgttaataatgattgacatttgtagaatt

SEQ ID NO: 146 - DNA, 119 bp, Bos taurus
  misc_feature (60)..(60): C OR A
ggaaaaatgtgtaatgatttgttggttacagttagagaaaagattggccccattgccacnccggattacatccagaatgcacctggattgcctaaaactcgctcaggtatgttcagagg

SEQ ID NO: 147 - DNA, 119 bp, Bos taurus
  misc_feature (60)..(60): G OR A
tttggaaaagaatacacatcctaggccctgctagagattctgattcaagaggtctaagcngagggaagagctgaacctctctttttaaaataaatattcctggaatgggaaccactgca

SEQ ID NO: 148 - DNA, 119 bp, Bos taurus
  misc_feature (60)..(60): A OR G
agacatttatgtggctccttaatggaggaactgatgatggagggactgatggaagatcangttgaaactcttccgtgagctctgtaaggttcctagtgttcctagttcttggtcgtctt

SEQ ID NO: 149 - DNA, 119 bp, Bos taurus
  misc_feature (60)..(60): T OR C
gtttgtgattgaaattcagagaaagaattttgtggttaccagggtaagaaaacatcaagngggcgactgaagttttttcttctcatgctaacaagataaagctgattaactctgtgatg

SEQ ID NO: 150 - DNA, 119 bp, Bos taurus
  misc_feature (60)..(60): G OR A
ctccgtccctccgttcagcactgggggctgcctagcttcctgcaccatcctgtcagcacnctcaaccctgccctgggccagcttctctaggtaaagtctgtggcaaaaacagaacataa

SEQ ID NO: 151 - DNA, 119 bp, Bos taurus
  misc_feature (60)..(60): C OR T
cttgccaaagtattataggttgtcagcgatcaaggagcttgtttttaagttcctgagganactgcaggcccccagtcaggatctttctattcaaactaattgtgaactgaacaatgaac

SEQ ID NO: 152 - DNA, 119 bp, Bos taurus
  misc_feature (60)..(60): G OR A
ctcactcggaacatcttacaattaatttttcctctcctaataatttctcaaaactccatntccttcaggtctagaccccagaaggctccttgactattccaggctgccccatggcccca

SEQ ID NO: 153 - DNA, 119 bp, Bos taurus
  misc_feature (60)..(60): G OR A
gtgtgtgtgtgtgtgtgtgtatgagaattcaatctgtgttgagaagatctatggcacagnaagagggaagttgaagtccaaatacctgttgcttgtaattcagtgaaatcaattagtgg

SEQ ID NO: 154 - DNA, 119 bp, Bos taurus
  misc_feature (60)..(60): G OR A
tccgaagagggaagacttttccttaattcgtatggtaatttggtccttttcaagaccctnacacaattctgcttcttatatgtgcgtaaggcatttttcaattgatgtgaaattctccc

SEQ ID NO: 155 - DNA, 119 bp, Bos taurus
  misc_feature (60)..(60): C OR T
tggatcagtagcaaaaaatatagtaccattaatttgactttttattaatgcgtcatgccngaaaatggtcctcagttatgcagggattccattgctggctttcctctgcatgcccccat

SEQ ID NO: 156 - DNA, 119 bp, Bos taurus
  misc_feature (60)..(60): T OR C
gaggagaataccaatttgataaaagctgtgtgtgctgaaaaagcccagaaattgctgttnagcagattggagcccgatcgttccattattcctgggctctgcgtgtcctcagctgattc

SEQ ID NO: 157 - DNA, 119 bp, Bos taurus
  misc_feature (60)..(60): G OR A
ctgatgagaaattgcctggtgtgggggagctgctggattgccagtgaggaagaggtcccnactggaacggatatgtggaccctcagaggagccccaggaagcccttcactgcagcaaac

SEQ ID NO: 158 - DNA, 119 bp, Bos taurus
  misc_feature (60)..(60): T OR C
tcagccttttccccaagcaaggggagccgcaggcggccagcaggactgggaattcaactngctgccaccgtgacagcaggcgtgttgggggaagtctcttggtgacaagtaagtgggca

SEQ ID NO: 159 - DNA, 119 bp, Bos taurus
  misc_feature (60)..(60): G OR A
ggcccatgttcccaatgctgttgtgtcccagagaggagggagaggcccttggtcccatgntgatgacctctacattccacacccgagctgtgcagcaggggacctggccacccccagac

SEQ ID NO: 160 - DNA, 119 bp, Bos taurus
  misc_feature (60)..(60): A OR G
ggtctcgctagggggataaatccactcctttaccagctggtgaccccaggatcgctctgntacctgctgctgtggagaaggggggtgggcagaggggctgctgctgctccaggtccccc

SEQ ID NO: 161 - DNA, 119 bp, Bos taurus
  misc_feature (60)..(60): G OR A
tctgggtggcttggcaggtccttgcccctctctgggcctcattttcccacctgaacagcnccaagttctcctaagctctaagcatctagaacacctccatctgtatcctcccaagtgct

SEQ ID NO: 162 - DNA, 119 bp, Bos taurus
  misc_feature (60)..(60): G OR T
cagccccctccaggcaggtgcccttgggacgtgaggtgtggaggctctgggagcatctgnaaggttagagctggagccaggaggggggcctcgttcagagctatcaattagggggctgg

SEQ ID NO: 163 - DNA, 119 bp, Bos taurus
  misc_feature (60)..(60): C OR T
tgaatgaccacacggggaagctaccaggggcgctggaccttgtctagactaatggttccnagttttttttggaggtcacgatcctcttgagaattggactggttgatgaaaactgggtt

SEQ ID NO: 164 - DNA, 119 bp, Bos taurus
  misc_feature (60)..(60): A OR C
acatacttgagtcagacactcactttataaaaacactaatggatttaatccttacaacangggcatagatgtaaacaacagctgattccctttgctgtatagcagaaactaaccacatt

SEQ ID NO: 165 - DNA, 119 bp, Bos taurus
  misc_feature (60)..(60): A OR G
gtccctctttttcctctgggtcctctgccccttttgccaacacatcagccctccagtgcncagagcatggtctgaagagcctgaggctgcagagatgaaggcagcctccgattggatgg

SEQ ID NO: 166 - DNA, 119 bp, Bos taurus
  misc_feature (60)..(60): G OR A
cctcctttcaccctgatggaaacccatcgctgcccttccttccacccgtgagacccaccntaccatctcccatcctcctgtgggacggactcacccagccctaggaggagaagtgccat

SEQ ID NO: 167 - DNA, 119 bp, Bos taurus
  misc_feature (60)..(60): C OR T
agcctgcaagagcaagcctgttctcaggcctctcctccactggatgctaggagctcctcnccatccgctgagaccaaggagacagccaggattggcctacacaccatgagggactaacc

SEQ ID NO: 168 - DNA, 119 bp, Bos taurus
  misc_feature (60)..(60): C OR A
agatttgggtgcattttgtcattggaccctgctcccacgtcgttcagagtcagaccatgnaggaggctacaaaatgctgacggaatgggtgaacaggagcagcagtcatcacatctcat

SEQ ID NO: 169 - DNA, 119 bp, Bos taurus
  misc_feature (60)..(60): G OR A
gagttctgaacagtcttcccccacccggaacttctttccagtctgctcaggctctgcaancacagggaagaggcaatcttgaatttcaagtgtatttcattgaactcatctaatgattg

SEQ ID NO: 170 - DNA, 119 bp, Bos taurus
  misc_feature (60)..(60): G OR A
gtgcaggtgaacaagttacctagagacgtgcccatcgcttagtgctcaggaactgggacnggaccccacgatggttgttacccatgtgatggacaagattgaacacagaacaagcctgg

SEQ ID NO: 171 - DNA, 119 bp, Bos taurus
  misc_feature (60)..(60): C OR T
ggatatatatccagcagtggaagtagttctcttttttaggtttttgtgcctcatgctctnttctaagaaaggggtccctgctcagcccagcactcaagagttgagtcaggtagttggat

SEQ ID NO: 172 - DNA, 119 bp, Bos taurus
  misc_feature (60)..(60): T OR C
acaaaaaaacccgaaacctgtttgccagaaacttatagccacctgacccccggatgacgnggcatctggccatcagggctgtgttctgttggacctctgagaccccgtctacagggctc

SEQ ID NO: 173 - DNA, 119 bp, Bos taurus
  misc_feature (60)..(60): G OR A
ggggaacagatggaaccccagcctggtgttgcttctttatctcagagccttcggagggangtcagctggccatcgcccttaaaccagctggatcctgtctgatggggttgcctaccgtg

SEQ ID NO: 174 - DNA, 119 bp, Bos taurus
  misc_feature (60)..(60): T OR C
tctgtcaccccagaaagctcctacgttccctcttccaggcagcccttctaccccaaaganatgagtgtgtgcttagtcacttcagtcgtgttcaactctttgggaccccatggactgta

SEQ ID NO: 175 - DNA, 119 bp, Bos taurus
  misc_feature (60)..(60): C OR T
ggcagcagcgaaggcaaggcccccttgatcggaggcagaacttctccgtacagtagcaangggacctcggtgtattacacggtcaccagtggagaccccccactcctgaagttcaaggc

SEQ ID NO: 176 - DNA, 119 bp, Bos taurus
  misc_feature (60)..(60): A OR G
ctgctccccacagccttgcccatgcctagagccagctgtgtgagcacccaggggccaagntagaacaccaggtaattctgctcctggaataatccaaatctattttgctcttggatgga

SEQ ID NO: 177 - DNA, 119 bp, Bos taurus
  misc_feature (60)..(60): T OR C
tttcagtcagggataaaccattcccatcaagtcccagggaaactttccctgtggatctcnttggtgtcgagggacaagctggggccaaggtcattgagttattcactctgtagtttatt

SEQ ID NO: 178 - DNA, 119 bp, Bos taurus
  misc_feature (60)..(60): A OR G
gcagtacccaaggtggcgggggctggtagggtgtagcagacaccttcctccagagccatnatcaactgccaccttgcttgctaactggcagtgcccagcttcctggctgtttgcctgtg

SEQ ID NO: 179 - DNA, 119 bp, Bos taurus
  misc_feature (60)..(60): A OR G
tcagttgtattgacctgtggacctagctactccacgtgtgtatatctgtcccaaagacanatctgatacttaaattgctaattttgtaataacagcccatgaaacttagagaaaacaat

SEQ ID NO: 180 - DNA, 119 bp, Bos taurus
  misc_feature (60)..(60): T OR C
aagtttaaagaggaaaaagaaaaagtccagtccactgtgtactttatgctggaacagtanagaggaaacgccaccatatgtgcctggcccctcactcctggggccagtgaaaaactggt

SEQ ID NO: 181 - DNA, 119 bp, Bos taurus
  misc_feature (60)..(60): T OR C
tatcttctgtcctgttggctccatcactgcccttgtcactctttaaacctcttcctgtcngtgacctgtatgatcctgagccttgcttggcgtccaagaccttccatgctcttgcacac

SEQ ID NO: 182 - DNA, 119 bp, Bos taurus
  misc_feature (60)..(60): C OR T
ggcctcggaagcttaggcgtgcacagtggtcccgagggattcagggatccgcccccgacntgggcagaaattctctgtgacctcgttacagtctcagcctccccgggcctcgttttcct

SEQ ID NO: 183 - DNA, 119 bp, Bos taurus
  misc_feature (60)..(60): G OR T
aatgacctcacatttccaaacctcagatccctcatttgcaaaataatttttaaaatatcngctgaaacaagaaccatctgctatttcctttgtcagtaattttaacccagggtcatcct

SEQ ID NO: 184 - DNA, 119 bp, Bos taurus
  misc_feature (60)..(60): C OR T
aatagaacttatacagtagaggtaaaaaaaaaaaaatcactaaaaaagcccatagaatanggctttccagatgtctagtggtatgaccatacagaaaatttctaaaaaggaacttgaaa

SEQ ID NO: 185 - DNA, 119 bp, Bos taurus
  misc_feature (60)..(60): A OR C
tagaaccagagctcaaagctgtcctttctcgtgtgtttcactgtttctccccaccgatcnacgtagctgtctaaatatagaaacttagctataaaagacttaaaaacagggacaaatag

SEQ ID NO: 186 - DNA, 119 bp, Bos taurus
  misc_feature (60)..(60): T OR C
tgtgagttcccaggtgagagtaagagggcgaagaagaggagggaatgggctgaagtgganaagagaggaggagtgtgtgagagtggtgaatgggggggcgtggctcaggtagtattttc

SEQ ID NO: 187 - DNA, 119 bp, Bos taurus
  misc_feature (60)..(60): A OR G
acgttgtactcgattcaggttctcagggcacccctggatcctggtttccaggagcagacntggacgtgtttaggggagatacctgggaaggaaagaagacaggcctggtgatcccatgg

SEQ ID NO: 188 - DNA, 119 bp, Bos taurus
  misc_feature (60)..(60): A OR G
aaaattataagaataaaaatatgattttttgaataattattttgttatccttaatctgtnttacgtatagccaatgcctatccccccaccccctgctctgccagttttatattattttc

SEQ ID NO: 189 - DNA, 119 bp, Bos taurus
  misc_feature (60)..(60): G OR A
gttgcagggaggagggctctagaacggtccaaactgggctttacaagactgtcaactcgngtggtattggtgatttgaatcaattggttctcagccctctacatcttagcaggaaggcc

SEQ ID NO: 190 - DNA, 119 bp, Bos taurus
  misc_feature (60)..(60): T OR C
cactgggacaagagacccacttttcctggccagggaatctttgcatgctaaagataggantttggtttgcacgggctttattgcttttctgtctctgcaaataaagccaaaaatagtag

SEQ ID NO: 191 - DNA, 119 bp, Bos taurus
  misc_feature (60)..(60): T OR C
atcctgcacgaggcacatgctgcgcctctgtgtcgctgctgcacctttttcccagtgacntttgcagctcaccctggagggaacaggttggcacaccactggaaaaacaaccagacagg

SEQ ID NO: 192 - DNA, 119 bp, Bos taurus
  misc_feature (60)..(60): T OR C
cagctgacagtaatggggtacagcagatccagcctctaggtttcctccaccagtgcttcnagagatcacagctctgagcagggcgccacctcctctgacccttaacagtttcccagatc

SEQ ID NO: 193 - DNA, 119 bp, Bos taurus
  misc_feature (60)..(60): G OR A
tgggaggattttgtctataatccttctctaaatgatccatagactttctgaatatacacnaaaagggtttaatctcttcctccctgtttttttccctgaccctttttatctagcttctt

SEQ ID NO: 194 - DNA, 119 bp, Bos taurus
  misc_feature (60)..(60): G OR A
agacacaggaaaggcccgcagcccagttaagtcatgtctgaggcgagacctgaaacccangtgggattttagcgagtcaggaggggtggagtgatccaggagtgtgtcggcatgataaa

SEQ ID NO: 195 - DNA, 119 bp, Bos taurus
  misc_feature (60)..(60): A OR C
tgttctgtaattattcagaacctcattcagcgctcggtcattcctgttactcatgtagtntcggcgtaaagacaggggtcaagcccccttttaggtgctgtgggtatgtttcctcttcg

SEQ ID NO: 196 - DNA, 119 bp, Bos taurus
  misc_feature (60)..(60): T OR C
agtaaaggttcgtggaaagaaagaagacatgaggatccagagactcagactggaagtcanagtatcagctttgttaaacttcagattgtctagcattccctctatttccagaagttcaa

SEQ ID NO: 197 - DNA, 119 bp, Bos taurus
  misc_feature (60)..(60): A OR C
cagtcgatggttcaagcactggagttactctatgctctgggaggtacgtctgtctcttantctgtgtttcttacgtatttattagtatgtgatattaggttgtattcactctagggctc

SEQ ID NO: 198 - DNA, 119 bp, Bos taurus
  misc_feature (60)..(60): A OR G
gcggttctcattgattttattttgtcgggaaagttgtttctcaaattctgtgtaccttgngggagggggcacaatgtagcatgtgccagcattttttttaagcctgaaggagttttcat

SEQ ID NO: 199 - DNA, 119 bp, Bos taurus
  misc_feature (60)..(60): A OR G
tgtcctcactctcggtatagctgtgcagagagaggaatccaggtcccattccacagcccnacagagaacagatggactccagtacctggggagaaaggcttccgtagagggatgtattt

SEQ ID NO: 200 - DNA, 119 bp, Bos taurus
  misc_feature (60)..(60): G OR A
catgtcccgatgccactagcgaaagctcatcagctcgaaacccatcccagatcctcggtngcagaaattcacaaccgtttagaggctctaagcttctgtagctcaggaacataacttgt

SEQ ID NO: 201 - DNA, 119 bp, Bos taurus
  misc_feature (60)..(60): T OR C
cacttcccatttcattttacttctgttactcctttctcttagtttgcttaattggtgtcngtggttggcaaaaggcctaactgtaggtgtgagagcaggagaaaagatgtttgcaagat

SEQ ID NO: 202 - DNA, 119 bp, Bos taurus
  misc_feature (60)..(60): A OR G
agtggcaccatttgtgcagaaacacctgtatatatggagcgggagtgaatttgctggatncttgcagtaaagacagtgaggcctccttcctgctcgccctctctccctccctctctctt

SEQ ID NO: 203 - DNA, 119 bp, Bos taurus
  misc_feature (60)..(60): T OR G
tatgcagctgtattgtgctaacacctaactgatggtaactagcaaccctgcatggacacntagatagctgcacccacagtgaaagcatatccttcagatatacacattgagtgggtagc

SEQ ID NO: 204 - DNA, 119 bp, Bos taurus
  misc_feature (60)..(60): A OR G
catttaaaaaaacagtaggtgttcagttaaccctctgaaggtgtgaaggcatcttcctantccttccctaccctgcaactcaccttgggtggtgcacacaccccagcatcagtgcacgg

SEQ ID NO: 205 - DNA, 119 bp, Bos taurus
  misc_feature (60)..(60): T OR C
cctcagtcttcagaacagtttctcagtagagggatgctggaaaagaatggtgtttatttnagaggatgtcggtgaaggcagtggctcccgggcttacctgcacaagcgggatcatttgt

SEQ ID NO: 206 - DNA, 119 bp, Bos taurus
  misc_feature (60)..(60): A OR G
actgtgctaaccacattgtgtctattaacttacttgatcttgatgaaagctccgcagganggaatggaagtttgaaaaggtcaccgctttccagggatcacactccctggtttataaca

SEQ ID NO: 207 - DNA, 119 bp, Bos taurus
  misc_feature (60)..(60): A OR G
accccctgaggcccaaatggagagctaacacttctatgaagctactcccttcctgaaacngggtggaagcccttcttctgtggaatctcagagcactttctgtaccttgtatatttcat

SEQ ID NO: 208 - DNA, 119 bp, Bos taurus
  misc_feature (60)..(60): T OR C
acccacggtttcaaagacatgtagagacgtgaagacacatagatatacatatatgaacgngcatttcaaaacacacagattcacatggatatgtggacagaatacagacacacccccat

SEQ ID NO: 209 - DNA, 119 bp, Bos taurus
  misc_feature (60)..(60): T OR G
gactagggtcagggtctgtgggtggggaccaccctccccttcagagctcaggcaggcttntgatgtccttgagtgctaggggagtaggtaaaactgagttttctcatttttagcaaccc

SEQ ID NO: 210 - DNA, 119 bp, Bos taurus
  misc_feature (60)..(60): A OR G
atgcaacaggaatcctaggccatggccgaggtggttatcccttcatggaattcccttccngccctgatgtgcatctgtggaacccctggcatctggatgtagatgtcacctcttccctg

SEQ ID NO: 211 - DNA, 119 bp, Bos taurus
  misc_feature (60)..(60): G OR A
cttgggatggggcaataagcacagagacctcagctctggtctgggctccatcctgactcntttctagtctggtttgaagccagataagtcttatttcctctccctgagcatctcaagca

SEQ ID NO: 212 - DNA, 119 bp, Bos taurus
  misc_feature (60)..(60): A OR G
gcatgggaggcattgcagcttagaaatctatctttgctttcctttttgctgataagaacntgaactctctcctgttggtccctcatggaatgataagtggtggatccattccctcacaa

SEQ ID NO: 213 - DNA, 119 bp, Bos taurus
  misc_feature (60)..(60): C OR T
gcaaatgacaggggatgttacacaggttagtataatcctcagtgtgaaaatgatttgaantccagatcccaggcaggtatttgggactgtggatttcttcccatgctctcttttctgct

SEQ ID NO: 214 - DNA, 119 bp, Bos taurus
  misc_feature (60)..(60): T OR C
gctttacacagaaaaacaaaacaaaaacagaaaatagagataacagcttctatgggtacntcaagttctcttagaactgcatcaagggggaaaaaaattctcaccaaatatttgagtta

SEQ ID NO: 215 - DNA, 119 bp, Bos taurus
  misc_feature (60)..(60): C OR T
accctgaatcaggagttcatttacacagatgtctagaaactcccgcacatctacacacantcactcacattctttctaaatacatgttttatcttccttgttatcaatgctctcctaac

SEQ ID NO: 216 - DNA, 119 bp, Bos taurus
  misc_feature (60)..(60): C OR T
ccacgaactaggactctgatttgtgttggggcttcccttgtccctcatttctaaggagcntcctgtctggcagggccgggtttaggatttggaaatcaatgtcagaatggaagggccga

SEQ ID NO: 217 - DNA, 119 bp, Bos taurus
  misc_feature (60)..(60): C OR T
tcctcgcagaggcaggacgaagcctctccaggcatttctgagttgcccttcagggcagcncttttgtgatgtgaatgcaatgatgaaaggccccagatctgtggctctgggtggaatac

SEQ ID NO: 218 - DNA, 119 bp, Bos taurus
  misc_feature (60)..(60): G OR T
atttccatggggaagggacttccacatactctctgcagttgtctgcaagtctaagaaagnatggagggagggaagggctggggcctcaggctagagcatggtgctgatgaagttccagc

SEQ ID NO: 219 - DNA, 119 bp, Bos taurus
  misc_feature (60)..(60): C OR T
aaggccacgactagctgagacccactgctgccccaggctcccacgtacctgtgaagtagnggagaagatgccggggttccacagagccaccactcctcggcccagtatctaatgagccc

SEQ ID NO: 220 - DNA, 119 bp, Bos taurus
  misc_feature (60)..(60): G OR T
gccagaggcagggtcgggccccgcacgtccctgtccgtctcctccatcactattacagtnacggctctgggagagcactcaggcgtgaaaaagctccttagaaacactgggaactgagg

SEQ ID NO: 221 - DNA, 119 bp, Bos taurus
  misc_feature (60)..(60): T OR C
agaaaaacatgaaaatgatctaagttttaaaatttaagagaattaagccatgttaaaaanggaatgagcagacatcaagaagcctgctattcaggccccagtttatgatagctgatgca

SEQ ID NO: 222 - DNA, 119 bp, Bos taurus
  misc_feature (60)..(60): A OR G
aacttttttttttttaacacacactaaaatgtagtaggctgtgcacttaagatacctacnttcactatataatagactttagcatagaggaagaatagtcaagtcacgaaagcaatgct

SEQ ID NO: 223 - DNA, 119 bp, Bos taurus
  misc_feature (60)..(60): C OR A
acagagggaaactgctggctttattggctatgctctagagaagatgaactgatccaccanacaatcacaagtgctaaaattatcaagaacaattatgtaaagtttaaaaataaaatcaa

SEQ ID NO: 224 - DNA, 119 bp, Bos taurus
  misc_feature (60)..(60): C OR T
tcagaagccacagctggctcgtcagtgcgcgagtttcttcggcttggagttttggacttntcaattatctctttgggctcactgctaacagagggaagcacaaagtccaaagaggtgag

SEQ ID NO: 225 - DNA, 119 bp, Bos taurus
  misc_feature (60)..(60): A OR G
ccacctgcaagtggctcagatgctatgccaacatgattgctttttgtggtcggcgccgcngagatgagttcagagggtaccagagcggttactattgaccgtacattggtggggagagt

SEQ ID NO: 226 - DNA, 119 bp, Bos taurus
  misc_feature (60)..(60): A OR G
cggtggctccaacaatccataacaccttgcaagaaattgtagactggctaatgaaaatgntagtcagagaacaaagaaagctggagaataccaaaacaaagctaattaattttgttgca

SEQ ID NO: 227 - DNA, 119 bp, Bos taurus
  misc_feature (60)..(60): A OR G
agctgggcagttagaaatctggcatcagagagagagctgaattctatgatgctctgtgcngggagtgcagcctcttcaattctcaaaggttggaggtcaatacctgactgagaagcagg

SEQ ID NO: 228 - DNA, 119 bp, Bos taurus
  misc_feature (60)..(60): T OR G
atcctttaaacactgaacagtacctggtgtacataacaggcactcattagctatttactnactacatgatgaaaagtcatcttatcataaccttacaaagaatcataggagtttttcca

SEQ ID NO: 229 - DNA, 119 bp, Bos taurus
  misc_feature (60)..(60): T OR C
tcaaggaaacaaaatcacatctgatggcctcaagggttgtgtttttgaaatgaatcttgntgatctgcagaatgatgaagctgcctttagacaattcaagctaattacggaggatgttc

SEQ ID NO: 230 - DNA, 119 bp, Bos taurus
  misc_feature (60)..(60): G OR A
gaaaagggaatggcagggatatccatacagaggagacaccaatgcgaaagacctgtgacnggaagaagaaatgtatattcaaggaaccaaaagaatgctgaagaggctagaacagacag

SEQ ID NO: 231 - DNA, 119 bp, Bos taurus
  misc_feature (60)..(60): A OR T
tttcttcctgagtgagactgcagtctggtcttaagaaaatatacataaatgtactaatantcaacaggttcaatatcagtaaaggagctggtttttggcaatttttgttgcctctttgt

SEQ ID NO: 232 - DNA, 119 bp, Bos taurus
  misc_feature (60)..(60): G OR A
gcaagttaaaagagagcctagcaaaacacagttacagaacctcctgaatgattaaagagnttaaagtatgttggaaggtaagaagaaaataagtaaaatgagttcaaaatggtaaacaa

SEQ ID NO: 233 - DNA, 119 bp, Bos taurus
  misc_feature (60)..(60): C OR T
ccaacgtagtgggttattgtcaagatttaagtgagataacatgtggaacatacctagcanagtgccaggtccatgaaaaccctcaataaagcttattatctccttatcacgatcatcac

SEQ ID NO: 234 - DNA, 119 bp, Bos taurus
  misc_feature (60)..(60): C OR A
gaaatgcagatcttcttgaactcaccccagtgatatttccatgatgctactttatcctcncaaacctaccaaatcaattaatttttttctatttctgccacaagaaataaagctgactt

SEQ ID NO: 235 - DNA, 119 bp, Bos taurus
  misc_feature (60)..(60): G OR A
cgacttagcagcagcagtagcaacagcagtagcagctatattgctataatattgttaccnatttgtgactctattgttacactccccagaagtaaacacaatgtataagtttttgaaaa

SEQ ID NO: 236 - DNA, 119 bp, Bos taurus
  misc_feature (60)..(60): C OR A
aagaaacagttaacagcaaaaagtacttcatatatccatcacccaggaaacactgttacnttttctgactttttccatgttgttctatgtgctaagtcacttcagtcatgtccggctct

SEQ ID NO: 237 - DNA, 119 bp, Bos taurus
  misc_feature (60)..(60): C OR A
tacagatgctccctctgggcttctgtttcaccacaaactcctcatgtaacctctccctgnctagcccctcctcaatctcctttgcctgccgctaccatcttccttctgcatgtttttta

SEQ ID NO: 238 - DNA, 119 bp, Bos taurus
  misc_feature (60)..(60): T OR C
acccattgagccattggggaagccattaacaagtgaggtataagttaaatatttagttcnccactataatttcattttactgcatgctgtatcattgatttatgtattgcatatttgct

SEQ ID NO: 239 - DNA, 119 bp, Bos taurus
  misc_feature (60)..(60): C OR T
aaaatttttggtcaacccaatacatgaaagagaggtttagattctttatttagttttgantagcaacatgtaattttaactgaagcagttttaagtataaaattgcagaagaaatttta

SEQ ID NO: 240 - DNA, 119 bp, Bos taurus
  misc_feature (60)..(60): A OR G
ataaacctttttagatttcatcactgagattccaccaacaacttggcatttaataatggnatgctatgtactcactgcagatggtgaatgcagccatgaaattaaaagacacttactcc

SEQ ID NO: 241 - DNA, 119 bp, Bos taurus
  misc_feature (60)..(60): T OR G
ccatctcacggggacgggtgccatcagctcaatgggatcagtccctggccttcctggccncgggtgcaggctgcagattgccaccatgaccgtcctaaaatacggtgcacaggagaacg

SEQ ID NO: 242 - DNA, 119 bp, Bos taurus
  misc_feature (60)..(60): G OR A
agctgttttccagaatttaacacatcgtttaaccagacatattcttagcagcactcaagncgttccgtggagtgggtgtatttgcaaattctggtcacgcagagctgattcacaaccac

SEQ ID NO: 243 - DNA, 119 bp, Bos taurus
  misc_feature (60)..(60): T OR C
ctcatctgggtccctctttgggctcagagtgagtctggggagagcattcctcagggtgcngagttggggggagcatctcagggctgcccaggccagggtgggacagagagcccactgtg

SEQ ID NO: 244 - DNA, 119 bp, Bos taurus
  misc_feature (60)..(60): T OR C
tgtggggggcgctgggggctgaccagaaacccccctcctgctggaactcactttcctccngtcttgatctctaccagccttgaacgagaacaaagtccttgtgctggacaccgactaca

SEQ ID NO: 245 - DNA, 119 bp, Bos taurus
  misc_feature (60)..(60): A OR G
tttctctctagttgcagtatgagaacttctctcattgcggagtgcagtctctagaatgcntgagctcagtagctgcgtcgtgcaggcttagatgtgggatctttgttccctgaccaggg

SEQ ID NO: 246 - DNA, 119 bp, Bos taurus
  misc_feature (60)..(60): G OR T
tatttgttgaggtgtttttcgtaggagctgttcaacctgagtccgaggtcgtggatgacnccattcaggtactcctccgcgaggttcaggacctcctggggtgtgccgcccacaaatgc

SEQ ID NO: 247 - DNA, 119 bp, Bos taurus
  misc_feature (60)..(60): C OR T
caccagcgataacaggtaagacttgtggagcccctttctgcgtgccggacgccgtggctnctgcggtctgggtcgtgagcatgtcgtggacaatttttttcaatctttaaagaggcata

SEQ ID NO: 248 - DNA, 119 bp, Bos taurus
  misc_feature (60)..(60): C OR T
gagacagtcccctgcgtggctgtcttccccttatgtttgcagactccaaagagctgagcngactcattggaaaagatcctgatgctggaaaagattgatggcaggatgataagggggca

SEQ ID NO: 249 - DNA, 119 bp, Bos taurus
  misc_feature (60)..(60): A OR G
gaggttgaaattgagaatgtataggctgaatataactcagagtatttttttaactttttnttttatattggggtatagttgattaacagtgttgtgattgtctcacgtgaacaacaaag

SEQ ID NO: 250 - DNA, 119 bp, Bos taurus
  misc_feature (60)..(60): C OR T
gaatcccccaggaatgggattattctgcccagagggcatttgtgcccaagatctccaggnacccggcatggccaatgcacaaaaaagcagcctgtgttcccttcaggctccctcaaaca

SEQ ID NO: 251 - DNA, 119 bp, Bos taurus
  misc_feature (60)..(60): G OR A
gccatggctgacttgagcatattgggggacagtgtttccattcaccaaaataccgaaaancaggttcgtggggacaggaggagatgtgaagttcaaacagtatagccacaccgtgtttg

SEQ ID NO: 252 - DNA, 119 bp, Bos taurus
  misc_feature (60)..(60): C OR T
ccttcaattctggagtagccactgttggttgtgtaccaacatccattctttccctctaanttactaacagaacctgtttgttcagtgtgtgatgagcccaaagtaagaattttcacctc

SEQ ID NO: 253 - DNA, 119 bp, Bos taurus
  misc_feature (60)..(60): G OR T
caagaatacagttgtataagaatacataattttacacggggtttcctacattcacgattntctttgggagggccatttatgtggtcaggtgtgccattcattcattcactgtgctgcag

SEQ ID NO: 254 - DNA, 119 bp, Bos taurus
  misc_feature (60)..(60): A OR G
ttgccattcccttctcccggaaaatccatttgtgtaccctaactcaggcacacggaggcnccaggcctaagaggagctgggggtgggcaggagaaggcactcagggtgggagcaggtcc

SEQ ID NO: 255 - DNA, 119 bp, Bos taurus
  misc_feature (60)..(60): C OR T
caacctcaggtatttggaaaattaaatcttttgtgcaaagaggcatgtggtggtggtggnggtttggttgctaagtcgtgtccaactcttgcaacccagtggactgtagcctgccaggc

SEQ ID NO: 256 - DNA, 119 bp, Bos taurus
  misc_feature (60)..(60): T OR G
ccacacgtccattctctacatctgtgtctctattcccgccctgaaaatagatagacattnctccaagaaagatagacagatggccgagagacacaggaaaagatgctcaataacactaa

SEQ ID NO: 257 - DNA, 119 bp, Bos taurus
  misc_feature (60)..(60): T OR C
cactaaacgctttgtgccatgagtaagttatacagacaccacagcacatgtggaataagnccggctcctcctctcgtggaaatgtcaatatcaagtactgagcttgaccaagcaacaca

SEQ ID NO: 258 - DNA, 119 bp, Bos taurus
  misc_feature (60)..(60): C OR T
caccctgaagcgggtagggaggagttctatagtgctcagtgagcagggtgtggtcagctnatgcacagttctaggattggttgccattgaactgaagtttccagcgtcatcaaacttct

SEQ ID NO: 259 - DNA, 119 bp, Bos taurus
  misc_feature (60)..(60): C OR T
taatagaacaatcttcttactagtctttgccaccaggagataatgaatgaggaaatccantgtggggggcataatgttagacacctgagaaatgaaagaaatgtttcctgctgtatcag

SEQ ID NO: 260 - DNA, 119 bp, Bos taurus
  misc_feature (60)..(60): A OR G
ctgtagcacaaatcccaccaccttttccagtgagctaaccctgaattcttcactctgccntccttggctttccccaggaatgcccataaagtcatggcctaactttcccctcactgttg

SEQ ID NO: 261 - DNA, 119 bp, Bos taurus
  misc_feature (60)..(60): C OR T
ggaaacccttcagagcagagtcctcgaaggcatggatttcagctctgaggacaggcacgngcaaagtggggttttaattccagtgagaacttccccaaagcaacacgtgctgactcctg

SEQ ID NO: 262 - DNA, 119 bp, Bos taurus
  misc_feature (60)..(60): C OR T
gcatataaggactatgcagacttcacggtgctggtggtagtgacaagaacattcagatgntggccctttcataagccaactcagctctaaatgctgacatttccaagaatcatcccctt

SEQ ID NO: 263 - DNA, 119 bp, Bos taurus
  misc_feature (60)..(60): G OR T
catcattgtaagcacagaaagcaaatattaaacaaagggaacctacagaaacaatcaganatcctctccctttgtgatatccatcacttcagaataaatattaataaacaaacctaagg

SEQ ID NO: 264 - DNA, 119 bp, Bos taurus
  misc_feature (60)..(60): C OR A
cagcagattagatgatgagaggagagaagttggggtactgataccctggcttcttcctgntggtgatggtggctgagcttcctgccctgtgagcatggctcctgtggtccagccctgtc

SEQ ID NO: 265 - DNA, 119 bp, Bos taurus
  misc_feature (60)..(60): C OR T
tgaaacttctctactcaaacttcatgctggtgcctctgtggcaactttggccaccaaggntggctgaggggtagggaagctggcatggatttaaaggcctcgagactaggggtgacccc

SEQ ID NO: 266 - DNA, 119 bp, Bos taurus
  misc_feature (60)..(60): G OR A
cgtgagcctcagggtaaggtggacatcttattccacttcattccactcccccccagttcntggatggaagggaagacccataatgggtgtgaacatcagtctacaaagtcattattttc

SEQ ID NO: 267 - DNA, 119 bp, Bos taurus
  misc_feature (60)..(60): G OR A
tgcacacacatacagaaactattaaccagtgaagtttcagctgccaaactgggaatgatntgcacaatacattcctggatgtgatggagctacaccctgtgagtattttgatcggcaag

SEQ ID NO: 268 - DNA, 119 bp, Bos taurus
  misc_feature (60)..(60): A OR G
atctgaaggcttgaccagggctggagggtccattttcaagttggctcttccatgcgactngccaaatagtgctggctgttggcaggagacctcagctcctcgccttgtagacctgccca

SEQ ID NO: 269 - DNA, 119 bp, Bos taurus
  misc_feature (60)..(60): A OR G
caaagactgcccatatgaaggattccacacaggacagaaatggctgaccctgtgcccccnccacactcaggcactgcctgggggctgcctctgctgaaaagcagagatggagactagat

SEQ ID NO: 270 - DNA, 119 bp, Bos taurus
  misc_feature (60)..(60): T OR C
gcaggtggaggcagcaggatggggaaagggaaacaaggctggagggctgagtctagaaanaagtctggggtcaaggggaaggaccggggtggcagtggagagtctcaatttgctgtgct

SEQ ID NO: 271 - DNA, 119 bp, Bos taurus
  misc_feature (60)..(60): A OR G
tctctgtggtttttctactctcttctcttcctcagagtccccagcacagttctctcagcngcattgaccactggagcctgagatgttggcagatagccctttgccagcaggtaggatcc

SEQ ID NO: 272 - DNA, 119 bp, Bos taurus
  misc_feature (60)..(60): T OR C
agtgggtgagagggtttgcctgcaggggcggcctggccgctgggctctggggttttctgngccctcctgaggcggctgtgtttggcactgaccatcgacggaacttattttcagcaggt

SEQ ID NO: 273 - DNA, 119 bp, Bos taurus
  misc_feature (60)..(60): A OR G
taggggcccacccactctctacagtgttgactaacctgcttaagtgaagttgctcagtcntgtccgactcttctcaaccccatggactgcagcccaccaggctcccccatgggattttc

SEQ ID NO: 274 - DNA, 119 bp, Bos taurus
  misc_feature (60)..(60): C OR A
aggttatagcccggcctcttcagggagatggttgttgatgtttattatcatcattttatntccattttacagaaaacacacatgagactcggggcatttaaagaacttgcctgtggtta

SEQ ID NO: 275 - DNA, 119 bp, Bos taurus
  misc_feature (60)..(60): T OR C
tgatgacaaagggatctggcttttagggtttaagagtgcagcatgatcctcacaatcctnggggcttgggatgaggcctttggactcagtggaagggtgaggacctcagggacccaggt

SEQ ID NO: 276 - DNA, 119 bp, Bos taurus
  misc_feature (60)..(60): C OR T
caggtgtagcggtaccatgcgtccttctgggctggcaaccctagagcagggtccaagaancctaagcacaggctgctgccatgtcccctccctacccatgttcaagggctctcagagta

SEQ ID NO: 277 - DNA, 119 bp, Bos taurus
  misc_feature (60)..(60): A OR G
gatccatgctgccctgcgaacaggggagaagcacaactttcttatttgcaggctgctgcnttatatgtttacctggccccagagagggcaagttggtccaggcactgagataccagagc

SEQ ID NO: 278 - DNA, 119 bp, Bos taurus
  misc_feature (60)..(60): C OR T
cactgcgcctgtcctccatgctgttgacaccctcacctgctgcatgatggcgccattttnattggaccccaggccttttaacacctgagagaccctctgcctggagccaggaaactgag

SEQ ID NO: 279 - DNA, 119 bp, Bos taurus
  misc_feature (60)..(60): C OR T
attcacagtgatgcaatgacgatgatgctttagcctgaaccacaaatcacaggtgacaangcaacattagctaaaaatatatctaattttttctgggttagatttacacctatcgcatt

SEQ ID NO: 280 - DNA, 119 bp, Bos taurus
  misc_feature (60)..(60): C OR T
tcttgatgatagctcttcttactgaatatgaggtaagatacctcactgtggtttcgtttngcatttctctaataatcagtgatatggagtatctttttatgtgtctttggccacctgtg

SEQ ID NO: 281 - DNA, 119 bp, Bos taurus
  misc_feature (60)..(60): A OR C
gtctttccactgtttataagcaaatataccccctatttttaggtaactggtagcatactntactggagaaggaaatggcaacccattccagtgttctcgcctggagaatcctgtggaca

SEQ ID NO: 282 - DNA, 119 bp, Bos taurus
  misc_feature (60)..(60): A OR G
atactagaacagaaacttcagtgaccacacatgatgagtaatatcatctttgcaaaaatngtttggaaaagtcactaagcaaatggatgactatattcctcaacaagcaacaacagcaa

SEQ ID NO: 283 - DNA, 119 bp, Bos taurus
  misc_feature (60)..(60): C OR T
cacactttctgtgcactctctcacaggtgctgccctgagactcttggccctctgttctcnccccaccgccctgtcctagaccattggacccacgccatcagacttcacttaccacaaga

SEQ ID NO: 284 - DNA, 119 bp, Bos taurus
  misc_feature (60)..(60): C OR T
aaagaaaaaactagaacattttcatgagcctgggtcctgccactttgggcatgtttgccngtcatttgccccgccaccccacacacctttaaccacctacctcacagacacccagcggc

SEQ ID NO: 285 - DNA, 119 bp, Bos taurus
  misc_feature (60)..(60): C OR T
tcctttgtcactcagccggctagtcactgctaactggatgactcttctctttccaggccnccagtcctcgctctgtggaggcagagcctgggtctcacctttactgtcttgtccccagc

SEQ ID NO: 286 - DNA, 119 bp, Bos taurus
  misc_feature (60)..(60): T OR G
caaacacaatcccacccgatgcggttttgattcttggcgcagaataagcgccctcggcgntgggatttatgcttcactcgcagtcctccgacctcggactctgcccagatgccgggaac

SEQ ID NO: 287 - DNA, 119 bp, Bos taurus
  misc_feature (60)..(60): T OR C
ccgccgcaaaccaaactccgcaccctttaaccctgtgtaccaccaagttgggctctgggngtgatgttggggggagagtggacaaggagagaggggtgggaggcagcacagtccctcca

SEQ ID NO: 288 - DNA, 119 bp, Bos taurus
  misc_feature (60)..(60): T OR G
aaatatttcgtattagtggctgcttctcttttctaaatgaaggcggccccttctgagggngtaagaggagggttggagtgaaggtgaatacctgccaggtccttcaggggagagccaac

SEQ ID NO: 289 - DNA, 119 bp, Bos taurus
  misc_feature (60)..(60): C OR T
tctggctccaagagacatgtcctggcccctacttcctcacacacttggtgttggcttggngtgctacaggtgttgtacctgggctggtgggagggatgggtagatatttcttcttgggt

SEQ ID NO: 290 - DNA, 119 bp, Bos taurus
  misc_feature (60)..(60): G OR A
gggcatagagagaaaacaaatgcctcttttttgttgtttttattaatgagcctccctacncaaatttttctttttgtattttactgccaggctggcaattacaagaagacatttgtctc

SEQ ID NO: 291 - DNA, 119 bp, Bos taurus
  misc_feature (60)..(60): T OR C
gacgccacagatgtccttctccccaagggaacagtgggcaattggaagcgcccccagganggtaggaatgttattctgacaaaatcgctctttctgcaggaggagctgtgccagtcgat

SEQ ID NO: 292 - DNA, 119 bp, Bos taurus
  misc_feature (60)..(60): G OR A
tggagaaccactaaacatttccacagtggctgagcactttccactcccagaaggtgcacnagggcccccgtgtctcacgtcctcaccagcagcaactgctttctgtctttttgactcta

SEQ ID NO: 293 - DNA, 119 bp, Bos taurus
  misc_feature (60)..(60): T OR C
aaaggcctcagatactgcagctgttggaggagaggtgggtgccctctggtgctctgcatnctgtgccgggacttgtctggggtgcagtcttggccagtggggagcctcccctccctccc

SEQ ID NO: 294 - DNA, 119 bp, Bos taurus
  misc_feature (60)..(60): G OR T
tgaagggcctcgaagggagaatacgcagcccaggacgctgtctggcccagtgaacacgangggggccactgtgccttggcagaggcaaacatgtgaggagtgtctttaggggctggtga

SEQ ID NO: 295 - DNA, 119 bp, Bos taurus
  misc_feature (60)..(60): G OR A
gacaccacttccaagggatcagctagcttcctttgccaacattgggacagagggacatcntgagccaatggacaggagagggatagcctcactcctgactcccaagtatgagattttgg

SEQ ID NO: 296 - DNA, 119 bp, Bos taurus
  misc_feature (60)..(60): G OR A
gctgtgcgactttccaaaggggtcgtcatgtcagccgcctggggcctgtgcagccccatntgcgaattcccactccaggtctcccaccagtcctggtcctgtgcccttgagagaccctg

SEQ ID NO: 297 - DNA, 119 bp, Bos taurus
  misc_feature (60)..(60): T OR C
ataattggagcctgtgatgttttcttaatccagtacacagctgggattctttagagtgtnggttatcaggatcttaacatctgaccattttggaagactttattttggagggcaagtaa

SEQ ID NO: 298 - DNA, 119 bp, Bos taurus
  misc_feature (60)..(60): G OR A
ccagttctaactgttgcttcttgacctgcatagtcctgtaaagcaaatagtccaagtccnctcctgttgcctcttgacatgcaagtcagataaagcagaagagaggaaacctttttata

SEQ ID NO: 299 - DNA, 119 bp, Bos taurus
  misc_feature (60)..(60): G OR A
cgcaatccatgttgccagtcaaatctaagtagaaacaagaagacaccctttgaacctacncaaattattatattgccaggaaaggaagaatactcaacaagagttaacaagttctggag

SEQ ID NO: 300 - DNA, 119 bp, Bos taurus
  misc_feature (60)..(60): G OR A
gaaagaagaaatgcaggatcactgaggcctaggcaccctgggctgggcctcacctgggcnggtctgctggaacccgataggctttgttccagactcctctcatgtgtgtgcgtgtgcac

SEQ ID NO: 301 - DNA, 119 bp, Bos taurus
  misc_feature (60)..(60): C OR A
tcgtgcaaagacagttgttgccggtatttagggagggctgcccacacttagagcgtctgntatagctccagggtccttggcctgggcctggccacgcgcatcagggctgctgtgccgct

SEQ ID NO: 302 - DNA, 119 bp, Bos taurus
  misc_feature (60)..(60): C OR T
gctggccatgtggatctttgttgcagggctttctgagctgctgttctcagacccttgagngggccagatggaggagggagttcatgaagccagggttgggaagcagctgggtctccagc

SEQ ID NO: 303 - DNA, 119 bp, Bos taurus
  misc_feature (60)..(60): C OR T
ctggcacgtagactccagtccacacctcatctttgccagctccctgtctccgtgcagganctcccagggggcctggagtccaggcccacaaactgaggctaggacagcccaccttgaat

SEQ ID NO: 304 - DNA, 119 bp, Bos taurus
  misc_feature (60)..(60): A OR G
gtttctccctggtcacaagaagttctacgcaactggcccagcatggaggaagtgttagcntagctcggggtgtttgagagagaggaggtcctgggggttacagagcggggctgggaaat

SEQ ID NO: 305 - DNA, 119 bp, Bos taurus
  misc_feature (60)..(60): A OR G
gagagggatgattctggggtaatcgctcttcctgtctctgggtgagctcagctccaggangcattgggaccagccggcagcggcaggggtggccagggagtgtggacctccggtcgcca

SEQ ID NO: 306 - DNA, 119 bp, Bos taurus
  misc_feature (60)..(60): T OR C
ctgagctgttcccctgctttgaaccaactgtcaccatctcatctcagctcatttttaggnatcaagtctacaactgtgccacaggacctctaggcaatttgggaagaaaaaccagaatg

SEQ ID NO: 307 - DNA, 119 bp, Bos taurus
  misc_feature (60)..(60): T OR C
cttccactctctctcagtcagttctcattgtctgcagggggcatgttctctcaagtcgcnaggacccctgaattagccaggcctggaccactgctccccagcaaagtgctgctcagggt

SEQ ID NO: 308 - DNA, 119 bp, Bos taurus
  misc_feature (60)..(60): A OR G
tggcgaacgactttgcgaacgatctggaaaagtgagagggagggtaggagggggcttggngaagccgtcgaggaagagcttgaaagggggcaggtcccagagggtggtgcagtgggtgc

SEQ ID NO: 309 - DNA, 119 bp, Bos taurus
  misc_feature (60)..(60): T OR C
ccgccccagctgagctcaggctgtatcaggcaaagtaaggaccgccacgtctggaactgnagcaggtacccgaaccgtcccatctgagccgggcttaccctgctgttagggtagtaaga

SEQ ID NO: 310 - DNA, 119 bp, Bos taurus
  misc_feature (60)..(60): C OR T
taaagggcgttttgaacaacgctgcccccacccccaccaccgagagcccgcccctatgcnagccagaggcagctttagctgtgcccaggaaaagggtcccatttaggactcgggaaaaa

SEQ ID NO: 311 - DNA, 119 bp, Bos taurus
  misc_feature (60)..(60): T OR C
tgaccgcacaccgtcctttggctttattctttgtgtgtgcatacgtgctccgttgtgtcngactctttgcagccccaaggactgcggcctgccaggctcctctgtccatggaattctcc

SEQ ID NO: 312 - DNA, 119 bp, Bos taurus
  misc_feature (60)..(60): C OR G
gtcctttggctttattctttgtgtgtgcatacgtgctccgttgtgtccgactctttgcanccccaaggactgcggcctgccaggctcctctgtccatggaattctccaggcaagaagac

SEQ ID NO: 313 - DNA, 119 bp, Bos taurus
  misc_feature (60)..(60): C OR T
attctttgtgtgtgcatacgtgctccgttgtgtccgactctttgcagccccaaggactgnggcctgccaggctcctctgtccatggaattctccaggcaagaagactggagtgggttgc

SEQ ID NO: 314 - DNA, 119 bp, Bos taurus
  misc_feature (60)..(60): A OR G
cccacatctctttcatctcctacattggtaggcagattctttagcactgagccgcctggnaagctgaagaaaactacaccaggacatgaatagcaaattattaaaaaccagcaacagag

SEQ ID NO: 315 - DNA, 119 bp, Bos taurus
  misc_feature (60)..(60): TA OR T
agcactgagccgcctgggaagctgaagaaaactacaccaggacatgaatagcaaattatnaaaaccagcaacagagaaaaaattcaggggaaaaaaaggaacaaagttaaggatggcag

SEQ ID NO: 316 - DNA, 119 bp, Bos taurus
  misc_feature (60)..(60): GC OR G
ttttaagaatgatttttgctggaaattgtggttttatacattccattaggttaaagaaanccttctgtttctatttaaataaaaggttttgttttggcttgcttttaaccaagaattgc

SEQ ID NO: 317 - DNA, 119 bp, Bos taurus
  misc_feature (60)..(60): A OR G
aaattgcattaatacgttttataacatcagatgatccttgcattgttggggaaaaaacantcacgatggattgttatccagtgctgatttgattttctactcacgccctggggtgcatt

SEQ ID NO: 318 - DNA, 119 bp, Bos taurus
  misc_feature (60)..(60): T OR C
tgcattaatacgttttataacatcagatgatccttgcattgttggggaaaaaacagtcangatggattgttatccagtgctgatttgattttctactcacgccctggggtgcatttacc

SEQ ID NO: 319 - DNA, 119 bp, Bos taurus
  misc_feature (60)..(60): C OR T
gtgacagagctcgagtcaaacatattagaagtaatatcatatggatgtcaatttaaagangtgccatattcttttttaccagcctctggttctttccacagaaggaattactgcaggag

SEQ ID NO: 320 - DNA, 119 bp, Bos taurus
  misc_feature (60)..(60): A OR T
tcaaacatattagaagtaatatcatatggatgtcaatttaaagatgtgccatattctttnttaccagcctctggttctttccacagaaggaattactgcaggagctattaaactaaaga

SEQ ID NO: 321 - DNA, 119 bp, Bos taurus
  misc_feature (60)..(60): T OR G
taaagatgtgccatattcttttttaccagcctctggttctttccacagaaggaattactncaggagctattaaactaaagacaccgctactgctatataaatgttccgtagttattaaa

SEQ ID NO: 322 - DNA, 119 bp, Bos taurus
  misc_feature (60)..(60): T OR G
taaactaaagacaccgctactgctatataaatgttccgtagttattaaaataattatacntttaatagttgcattattcagttcagttcagttcagtcgctcagtcatatccaactctt

SEQ ID NO: 323 - DNA, 119 bp, Bos taurus
  misc_feature (60)..(60): C OR A
ttatacgtttaatagttgcattattcagttcagttcagttcagtcgctcagtcatatccnactctttgcaaccccatggaccgcagcatgccaggcctccctgtccatcaccaactccc

SEQ ID NO: 324 - DNA, 119 bp, Bos taurus
  misc_feature (60)..(60): A OR G
actctttgcaaccccatggaccgcagcatgccaggcctccctgtccatcaccaactcccngagtttatccaagctcatgtccattgagtcggtgatgccatccaaccatctcatcttct

SEQ ID NO: 325 - DNA, 119 bp, Bos taurus
  misc_feature (60)..(60): G OR A
agttctttgcatcaggtggccagagtattggagtttcagcttcaacatcagtccctccantgaatattcagggcttgtttcctttaggatggactggttagatctccttgcagtccaaa

SEQ ID NO: 326 - DNA, 119 bp, Bos taurus
  misc_feature (60)..(60): A OR G
aggtggccagagtattggagtttcagcttcaacatcagtccctccaatgaatattcaggncttgtttcctttaggatggactggttagatctccttgcagtccaaaggactctcaagag

SEQ ID NO: 327 - DNA, 119 bp, Bos taurus
  misc_feature (60)..(60): C OR A
ggttagatctccttgcagtccaaaggactctcaagagtcttctccaacaccacagtttcnaaagcatcaattcttcagcacttagctttcttcacagtccaactctcacatccatacat

SEQ ID NO: 328 - DNA, 119 bp, Bos taurus
  misc_feature (60)..(60): G OR T
acaccacagtttcaaaagcatcaattcttcagcacttagctttcttcacagtccaactcncacatccatacatgactactggaaaaaccatagccttgactagatggacctttgttgac

SEQ ID NO: 329 - DNA, 119 bp, Bos taurus
  misc_feature (60)..(60): A OR T
tcaattcttcagcacttagctttcttcacagtccaactctcacatccatacatgactacnggaaaaaccatagccttgactagatggacctttgttgacaaagtattgtctctgtgttt

SEQ ID NO: 330 - DNA, 119 bp, Bos taurus
  misc_feature (60)..(60): C OR T
ctactggaaaaaccatagccttgactagatggacctttgttgacaaagtattgtctctgngtttttaatatgctgtctaagttggtcataacttttcttccaaggagtaagtgtctttt

SEQ ID NO: 331 - DNA, 119 bp, Bos taurus
  misc_feature (60)..(60): G OR A
attgtctctgtgtttttaatatgctgtctaagttggtcataacttttcttccaaggagtnagtgtcttttaattttatggctgcagtcaccatctgcagtgattttagagccccccaaa

SEQ ID NO: 332 - DNA, 119 bp, Bos taurus
  misc_feature (60)..(60): C OR T
gtctctgtgtttttaatatgctgtctaagttggtcataacttttcttccaaggagtaagngtcttttaattttatggctgcagtcaccatctgcagtgattttagagccccccaaaata

SEQ ID NO: 333 - DNA, 119 bp, Bos taurus
  misc_feature (60)..(60): A OR C
agtaagtgtcttttaattttatggctgcagtcaccatctgcagtgattttagagcccccnaaaataaagtcagccactgtttccactgtttccccaactatttgccgtgaagtgatggg

SEQ ID NO: 334 - DNA, 119 bp, Bos taurus
  misc_feature (60)..(60): C OR G
tagagccccccaaaataaagtcagccactgtttccactgtttccccaactatttgccgtnaagtgatgggaccggatgccatgatcttagtttcctgaatgttgagctttaagccaact

SEQ ID NO: 335 - DNA, 119 bp, Bos taurus
  misc_feature (60)..(60): G OR C
gatgggaccggatgccatgatcttagtttcctgaatgttgagctttaagccaactttttnactctcctctttcactttcatcaagaggctctttagttcctcttcactttctgccataa

SEQ ID NO: 336 - DNA, 119 bp, Bos taurus
  misc_feature (60)..(60): G OR A
atgggaccggatgccatgatcttagtttcctgaatgttgagctttaagccaactttttcnctctcctctttcactttcatcaagaggctctttagttcctcttcactttctgccataag

SEQ ID NO: 337 - DNA, 119 bp, Bos taurus
  misc_feature (60)..(60): A OR C
accggatgccatgatcttagtttcctgaatgttgagctttaagccaactttttcactctnctctttcactttcatcaagaggctctttagttcctcttcactttctgccataagtgtgg

SEQ ID NO: 338 - DNA, 119 bp, Bos taurus
  misc_feature (60)..(60): T OR C
atcttagtttcctgaatgttgagctttaagccaactttttcactctcctctttcactttnatcaagaggctctttagttcctcttcactttctgccataagtgtggtgtcatctgcata

SEQ ID NO: 339 - DNA, 119 bp, Bos taurus
  misc_feature (60)..(60): G OR T
cttagtttcctgaatgttgagctttaagccaactttttcactctcctctttcactttcancaagaggctctttagttcctcttcactttctgccataagtgtggtgtcatctgcatatc

SEQ ID NO: 340 - DNA, 119 bp, Bos taurus
  misc_feature (60)..(60): T OR C
agctttaagccaactttttcactctcctctttcactttcatcaagaggctctttagttcntcttcactttctgccataagtgtggtgtcatctgcatatctgaggttattgatatttct

SEQ ID NO: 341 - DNA, 119 bp, Bos taurus
  misc_feature (60)..(60): G OR T
ctctcctctttcactttcatcaagaggctctttagttcctcttcactttctgccataagngtggtgtcatctgcatatctgaggttattgatatttctcccggcaatcttgattctagc

SEQ ID NO: 342 - DNA, 119 bp, Bos taurus
  misc_feature (60)..(60): A OR G
tctcctctttcactttcatcaagaggctctttagttcctcttcactttctgccataagtntggtgtcatctgcatatctgaggttattgatatttctcccggcaatcttgattctagcc

SEQ ID NO: 343 - DNA, 119 bp, Bos taurus
  misc_feature (60)..(60): C OR T
gcccagcgtttctcatgatgtactctgcatataagttaaataagcagggtgacaatatanagccttgacgtactccttttcctatttggaaccaatctgttgttccatgtccagttcta

SEQ ID NO: 344 - DNA, 119 bp, Bos taurus
  misc_feature (60)..(60): A OR G
tctcatgatgtactctgcatataagttaaataagcagggtgacaatatatagccttgacntactccttttcctatttggaaccaatctgttgttccatgtccagttctaactgctgctt

SEQ ID NO: 345 - DNA, 119 bp, Bos taurus
  misc_feature (60)..(60): A OR G
ttaaataagcagggtgacaatatatagccttgacgtactccttttcctatttggaaccantctgttgttccatgtccagttctaactgctgcttcctgacctgcatacaggtttctcaa

SEQ ID NO: 346 - DNA, 119 bp, Bos taurus
  misc_feature (60)..(60): G OR T
tttttctggaactctcttgctttttcgatgatccagcagatgttggcaatttgatctctngttcctctgccttttctaaaaccaggttgaacatctggaagttcacagttcacgtattg

SEQ ID NO: 347 - DNA, 119 bp, Bos taurus
  misc_feature (60)..(60): A OR G
ttccagaatttaacacatcgtttaaccagacatattcttagcagcactcaagacgttccntggagtgggtgtatttgcaaattctggtcacgcagagctgattcacaaccacatttctt

SEQ ID NO: 348 - DNA, 119 bp, Bos taurus
  misc_feature (60)..(60): T OR C
cacgcagagctgattcacaaccacatttcttgctctgtggcaccagtcttttcccccagnctctcttctgagcttcgtgttatcctttgctgaatttcccaaggagaaggcggagaatc

SEQ ID NO: 349 - DNA, 119 bp, Bos taurus
  misc_feature (60)..(60): C OR T
tcttgctctgtggcaccagtcttttcccccagcctctcttctgagcttcgtgttatcctntgctgaatttcccaaggagaaggcggagaatctggcttcagaatgtgtgtttccctcaa

SEQ ID NO: 350 - DNA, 119 bp, Bos taurus
  misc_feature (60)..(60): C OR T
agaatgtgtgtttccctcaaagagacaccaagcagaccttgcatgaggactgtctcagcngagatacccgtcctgcccctggccgtgtttctggggtttgggccatagaccccgcaggg

SEQ ID NO: 351 - DNA, 119 bp, Bos taurus
  misc_feature (60)..(60): T OR C
tcaaagagacaccaagcagaccttgcatgaggactgtctcagctgagatacccgtcctgnccctggccgtgtttctggggtttgggccatagaccccgcagggcagacggggggctctg

SEQ ID NO: 352 - DNA, 119 bp, Bos taurus
  misc_feature (60)..(60): A OR T
tgcatgaggactgtctcagctgagatacccgtcctgcccctggccgtgtttctggggttngggccatagaccccgcagggcagacggggggctctggccctgggctcacctttgttccc

SEQ ID NO: 353 - DNA, 119 bp, Bos taurus
  misc_feature (60)..(60): T OR C
aaaccaaacttggatgctctcactcacacgcagtaaagccagtctcctgacgccagggtngtggtgaagaaaagggcagtgtttcctgggttgggaatattccctggagaaggaaatgg

SEQ ID NO: 354 - DNA, 119 bp, Bos taurus
  misc_feature (60)..(60): A OR G
attccctggagaaggaaatggcaatccactccagcactcttgcctggaaaatcccatggncggaggaacctggtaggctgcagtccacggggtcgcaaagagtcggacacgactgagcg

SEQ ID NO: 355 - DNA, 119 bp, Bos taurus
  misc_feature (60)..(60): T OR C
gaaggaaatggcaatccactccagcactcttgcctggaaaatcccatgggcggaggaacntggtaggctgcagtccacggggtcgcaaagagtcggacacgactgagcgacttcaattt

SEQ ID NO: 356 - DNA, 119 bp, Bos taurus
  misc_feature (60)..(60): A OR G
gggcggaggaacctggtaggctgcagtccacggggtcgcaaagagtcggacacgactgancgacttcaatttcacttttactttactgcaggtgccaagcaaggagtccaagtgctcag

SEQ ID NO: 357 - DNA, 119 bp, Bos taurus
  misc_feature (60)..(60): T OR C
tgggctacaggttatgtgggctacagggtatctgatcagcttgtggacattcttcttatnggttaacgttgaggtcatcgggggtcaccgtggtcagccttctagttccacctgatctg

SEQ ID NO: 358 - DNA, 119 bp, Bos taurus
  misc_feature (60)..(60): G OR GGT
tccacctgatctggggtccacctggtctgtgggcagcatatagttaccttcttccacctngtgtggggggtggttctcagtatctgcagaaaaactcacaggacatggctcaggatatt

SEQ ID NO: 359 - DNA, 119 bp, Bos taurus
  misc_feature (60)..(60): G OR T
ggtccacctggtctgtgggcagcatatagttaccttcttccacctggtgtggggggtggntctcagtatctgcagaaaaactcacaggacatggctcaggatattctccatagccctgg

SEQ ID NO: 360 - DNA, 119 bp, Bos taurus
  misc_feature (60)..(60): A OR G
ccatagccctggtttagtcgctaagtcgtgtctcactcttttgtgaccccatggactgtngcccaccaggctcctctgtccatgggattctccaggcaagaatactggagtgggttgcc

SEQ ID NO: 361 - DNA, 119 bp, Bos taurus
  misc_feature (60)..(60): G OR C
cttttgtgaccccatggactgtggcccaccaggctcctctgtccatgggattctccaggnaagaatactggagtgggttgccatagcccttgaggaggaattaaatgtcctggactttg

SEQ ID NO: 362 - DNA, 119 bp, Bos taurus
  misc_feature (60)..(60): A OR G
caccaggctcctctgtccatgggattctccaggcaagaatactggagtgggttgccatancccttgaggaggaattaaatgtcctggactttgcttaatggctaagctattgctgtatt

SEQ ID NO: 363 - DNA, 119 bp, Bos taurus
  misc_feature (60)..(60): A OR T
ggctcctctgtccatgggattctccaggcaagaatactggagtgggttgccatagccctngaggaggaattaaatgtcctggactttgcttaatggctaagctattgctgtatttactt

SEQ ID NO: 364 - DNA, 119 bp, Bos taurus
  misc_feature (60)..(60): A OR T
ctctttggaagtgcgtgaaagcttaggaggctaaagcttttttacagacaagaggcaggngtaggacatggggtgggggaaaagcctgttttggggagaccccatggggtcctgctcag

SEQ ID NO: 365 - DNA, 119 bp, Bos taurus
  misc_feature (60)..(60): G OR T
ctttggaagtgcgtgaaagcttaggaggctaaagcttttttacagacaagaggcaggtgnaggacatggggtgggggaaaagcctgttttggggagaccccatggggtcctgctcagtt

SEQ ID NO: 366 - DNA, 119 bp, Bos taurus
  misc_feature (60)..(60): C OR A
ggaaaagcctgttttggggagaccccatggggtcctgctcagtttcactaggacattggngagggcagtgactcctccctgccccaggacatcgctgaccatatttttcctgatttatt

SEQ ID NO: 367 - DNA, 119 bp, Bos taurus
  misc_feature (60)..(60): C OR T
atcgctgaccatatttttcctgatttatttgaactatagtcctgtaaacaaaccttcatnaaaactcaatcatgcatcctcctaactcacgcactcctgcgtctagtctgtccacaaac

SEQ ID NO: 368 - DNA, 119 bp, Bos taurus
  misc_feature (60)..(60): T OR G
aacaaaccttcattaaaactcaatcatgcatcctcctaactcacgcactcctgcgtctantctgtccacaaacacggaccctgctgggagatgaatgagctcatagccctgacctggag

SEQ ID NO: 369 - DNA, 119 bp, Bos taurus
  misc_feature (60)..(60): G OR A
cctggagcaattctatctcatctccctttttttatgtgttttctctcagcagccccaccntgcttttaagaaattaggagacaaagaaggagtgatgactctctaagccgctcagtcta

SEQ ID NO: 370 - DNA, 119 bp, Bos taurus
  misc_feature (60)..(60): C OR G
tccctactagttagatccagtccaaaccatgtgtgttgaggcacgtgtgttgcggtgaanttcaggaggccattctgacaattagcacagttgcccaggaaacagcagtgttctggacg

SEQ ID NO: 371 - DNA, 119 bp, Bos taurus
  misc_feature (60)..(60): G OR C
gtattgcagtccaggtaacacgcctgtctcatctgtgccctcttagccacttcttccgcnacactcaggaccaggatctcacctgctgcttagctgctcaggcttcagcatgtgcagca

SEQ ID NO: 372 - DNA, 119 bp, Bos taurus
  misc_feature (60)..(60): T OR C
tatcttaaatatctttactaagataatcttctcaaaaatctccaaaggctccttattgtntagtgaattaagtaaaatgtttctctgactttttgagatttccaaatacgtaaacctag

SEQ ID NO: 373 - DNA, 119 bp, Bos taurus
  misc_feature (60)..(60): A OR G
aaagtagtctggttggattcttttgcatgcagctgaagagttttccttacgaacaaagcnagtggaggtgatggaaccccacttgagctacttcaaatcctgaaagatgatgctgtgag

SEQ ID NO: 374 - DNA, 119 bp, Bos taurus
  misc_feature (60)..(60): C OR A
caaaattctccaagccaggcttcagcaatatgtgatccgtgaacttccagatgttcaagntggttttagaaaaggcagaggaaccagagatcaaattgccaacatctgctggatcatgg

SEQ ID NO: 375 - DNA, 119 bp, Bos taurus
  misc_feature (60)..(60): C OR G
aaaaggcagaggaaccagagatcaaattgccaacatctgctggatcatggaaaaagcaanagagttccagaaaaacatctacttctgctttattgactatgccaaagcctttgactgtg

SEQ ID NO: 376 - DNA, 119 bp, Bos taurus
  misc_feature (60)..(60): C OR T
agagttccagaaaaacatctacttctgctttattgactatgccaaagcctttgactgtgnggatcacaataaactctggaaaattctgaaagagatgggaataccagacctcctgatct

SEQ ID NO: 377 - DNA, 119 bp, Bos taurus
  misc_feature (60)..(60): G OR C
atctacttctgctttattgactatgccaaagcctttgactgtgtggatcacaataaactntggaaaattctgaaagagatgggaataccagacctcctgatctgcttcttgagaaacct

SEQ ID NO: 378 - DNA, 119 bp, Bos taurus
  misc_feature (60)..(60): C OR T
atcacaataaactctggaaaattctgaaagagatgggaataccagacctcctgatctgcntcttgagaaacctgtatgcaggtcataaagcaacagttagaactggacgtggaacaaca

SEQ ID NO: 379 - DNA, 119 bp, Bos taurus
  misc_feature (60)..(60): C OR T
tgcagccatgaaattaaaagacacttactccttggaaggaaagttatgaccaaactaganagcatattgaaaagcagagacattactttgtcaacaaaggtctgtctagtcaaggctat

SEQ ID NO: 380 - DNA, 119 bp, Bos taurus
  misc_feature (60)..(60): C OR T
ttatgaccaaactagatagcatattgaaaagcagagacattactttgtcaacaaaggtcngtctagtcaaggctatggtttttccagtggtcatgtatggatgtgagagtaggactata

SEQ ID NO: 381 - DNA, 119 bp, Bos taurus
  misc_feature (60)..(60): G OR A
ggagttggtgatggacagggaggcctggcatgctgcagttcatggggtcacaaagaatcngacatgactgaatgactgaactgaagagttttcccagcaccatttattgaagagactgt

SEQ ID NO: 382 - DNA, 119 bp, Bos taurus
  misc_feature (60)..(60): T OR C
ggtatatcagtatagtcagtatagatcagtatagatcagtgtcagtatagtcactttaanaatattaactcttccaatctgtgagcatgatatattttcccctctatatcatcttcaat

SEQ ID NO: 383 - DNA, 119 bp, Bos taurus
  misc_feature (60)..(60): C OR T
ggttagagtcattcctcggtattttattccttctggtacaattgtgaatgagataatttncttagtttctctttctgatagctcattgttagtgtatagagaagcaacagatttctatg

SEQ ID NO: 384 - DNA, 119 bp, Bos taurus
  misc_feature (60)..(60): C OR T
tatcctgctattttactgaattcacttattagctttttggtgacatctaaagattttctnaagaaaatggcatggtatggtatgacaagctgtcaagccacctgcaaacagtggtggtt

SEQ ID NO: 385 - DNA, 119 bp, Bos taurus
  misc_feature (60)..(60): C OR G
cggatttcttttatttctttttgtctgagtactgtgactaggattcccagtactgtattnaatgaaagtggcaagagtgtacttccttgccttatttttctggtcttagaggaaatgct

SEQ ID NO: 386 - DNA, 119 bp, Bos taurus
  misc_feature (60)..(60): A OR G
tttcttttatttctttttgtctgagtactgtgactaggattcccagtactgtattgaatnaaagtggcaagagtgtacttccttgccttatttttctggtcttagaggaaatgctctca

SEQ ID NO: 387 - DNA, 119 bp, Bos taurus
  misc_feature (60)..(60): A OR G
tttttgtctgagtactgtgactaggattcccagtactgtattgaatgaaagtggcaagantgtacttccttgccttatttttctggtcttagaggaaatgctctcaggttttcaccatc

SEQ ID NO: 388 - DNA, 119 bp, Bos taurus
  misc_feature (60)..(60): C OR T
ttgtctgagtactgtgactaggattcccagtactgtattgaatgaaagtggcaagagtgnacttccttgccttatttttctggtcttagaggaaatgctctcaggttttcaccatcaat

SEQ ID NO: 389 - DNA, 119 bp, Bos taurus
  misc_feature (60)..(60): T OR C
attatgatgtttgctatgggcttgtcatatgtggcctttattatatggaggtttattccntctatacccactttgttgagagtttgtatcataaaagtatgttgaattttgtcaaatgg

SEQ ID NO: 390 - DNA, 119 bp, Bos taurus
  misc_feature (60)..(60): T OR C
tgctatgggcttgtcatatgtggcctttattatatggaggtttattccctctatacccantttgttgagagtttgtatcataaaagtatgttgaattttgtcaaatggttttcctgcat

SEQ ID NO: 391 - DNA, 119 bp, Bos taurus
  misc_feature (60)..(60): A OR T
ccctctatacccactttgttgagagtttgtatcataaaagtatgttgaattttgtcaaanggttttcctgcatctattgagatgatgtttatttttcaattcattaatgatttttattc

SEQ ID NO: 392 - DNA, 119 bp, Bos taurus
  misc_feature (60)..(60): T OR G
tgtatcataaaagtatgttgaattttgtcaaatggttttcctgcatctattgagatgatntttatttttcaattcattaatgatttttattcttcaatttgttaatgtggtatatccca

SEQ ID NO: 393 - DNA, 119 bp, Bos taurus
  misc_feature (60)..(60): T OR A
caatgatattgacctaaggtttttttttttttttttgtaaagtttttgtctggttttagnatcagggtgatgctggcctcatagagagagtttagaagcatttccttctctttgatttt

SEQ ID NO: 394 - DNA, 119 bp, Bos taurus
  misc_feature (60)..(60): C OR T
gtctggttttagaatcagggtgatgctggcctcatagagagagtttagaagcatttcctnctctttgattttttggaatagtttgagtaggataggtattaactcttctttaaaggtct

SEQ ID NO: 395 - DNA, 119 bp, Bos taurus
  misc_feature (60)..(60): T OR C
cacccactgtgtgcccacgggccacagccagagaaaacccacagacggcaatgaagtccnagcacaaccagaaaaagaagttcggtagatacagctgtgaagccctctggtcctggact

SEQ ID NO: 396 - DNA, 119 bp, Bos taurus
  misc_feature (60)..(60): A OR G
catattttctatttcttcctggctcagtcttgggtttgtacatgtctaggaatgtatccntttcttctgggttgtcccttttattagacatgtgtgggggcacacagcaccgaccagtg

SEQ ID NO: 397 - DNA, 119 bp, Bos taurus
  misc_feature (60)..(60): G OR A
catgtgtgggggcacacagcaccgaccagtgagactcataccggcttcctggggccaggntgcgggccccaagcagcacggcatcctagactgtgtgaatgcccactgaccctgcccag

SEQ ID NO: 398 - DNA, 119 bp, Bos taurus
  misc_feature (60)..(60): G OR C
gtgtgggggcacacagcaccgaccagtgagactcataccggcttcctggggccaggatgngggccccaagcagcacggcatcctagactgtgtgaatgcccactgaccctgcccagccc

SEQ ID NO: 399 - DNA, 119 bp, Bos taurus
  misc_feature (60)..(60): G OR A
ccagccccacagtttcattctgagagaagtgatttcttgcttctgcacttacaggcccangacctgacctgcttctgaagagcaggggttttggcaggagggggagatgctgagagccg

SEQ ID NO: 400 - DNA, 119 bp, Bos taurus
  misc_feature (60)..(60): A OR G
acctgcttctgaagagcaggggttttggcaggagggggagatgctgagagccgatggggntccaggtctcctcccaggtcccactctctggggcagcgcttggaaaagattgtccagcc

SEQ ID NO: 401 - DNA, 119 bp, Bos taurus
  misc_feature (60)..(60): C OR T
agctgaaggttcctggaagttatgaatagctttgccatgaagggcatggtttgtggtcanggttcacaggaacttgggagaccctgcagctcggacgtcctgaggttggtggcaccctg

SEQ ID NO: 402 - DNA, 119 bp, Bos taurus
  misc_feature (60)..(60): T OR C
tgccatgaagggcatggtttgtggtcatggttcacaggaacttgggagaccctgcagctnggacgtcctgaggttggtggcaccctgatttcctaagctcgctggggaacggggtgcta

SEQ ID NO: 403 - DNA, 119 bp, Bos taurus
  misc_feature (60)..(60): C OR T
gggcatggtttgtggtcatggttcacaggaacttgggagaccctgcagctcggacgtccngaggttggtggcaccctgatttcctaagctcgctggggaacggggtgctacttctccct

SEQ ID NO: 404 - DNA, 119 bp, Bos taurus
  misc_feature (60)..(60): T OR C
ggacgtcctgaggttggtggcaccctgatttcctaagctcgctggggaacggggtgctanttctccctggctgacctccctctgctctgcatcacccagttctgagagcagagtggtgc

SEQ ID NO: 405 - DNA, 119 bp, Bos taurus
  misc_feature (60)..(60): C OR T
tgctacttctccctggctgacctccctctgctctgcatcacccagttctgagagcagagnggtgctgggggcacagcctctcgcatctgacacttgtgttcaaaccacccatgctggtg

SEQ ID NO: 406 - DNA, 119 bp, Bos taurus
  misc_feature (60)..(60): TG OR T
ttctccctggctgacctccctctgctctgcatcacccagttctgagagcagagtggtgcnggggcacagcctctcgcatctgacacttgtgttcaaaccacccatgctggtgttcgggg

SEQ ID NO: 407 - DNA, 119 bp, Bos taurus
  misc_feature (60)..(60): C OR G
agtggtgctgggggcacagcctctcgcatctgacacttgtgttcaaaccacccatgctgntgttcggggggccacctatggggaaggctcctcactgcaggggtgcccctgtcccctga

SEQ ID NO: 408 - DNA, 119 bp, Bos taurus
  misc_feature (60)..(60): C OR G
ggtgttcggggggccacctatggggaaggctcctcactgcaggggtgcccctgtcccctnagagatcagaagtcccagtctggatgtcgaatggccgagctccctccagaggctccagg

SEQ ID NO: 409 - DNA, 119 bp, Bos taurus
  misc_feature (60)..(60): C OR T
ggggaaggctcctcactgcaggggtgcccctgtcccctgagagatcagaagtcccagtcnggatgtcgaatggccgagctccctccagaggctccagggagggatccttgccccctccg

SEQ ID NO: 410 - DNA, 119 bp, Bos taurus
  misc_feature (60)..(60): C OR T
aaggctcctcactgcaggggtgcccctgtcccctgagagatcagaagtcccagtctggangtcgaatggccgagctccctccagaggctccagggagggatccttgccccctccgccgc

SEQ ID NO: 411 - DNA, 119 bp, Bos taurus
  misc_feature (60)..(60): T OR C
tccagaggctccagggagggatccttgccccctccgccgccgcctccagctcctggtgcngcacccttgggcccgatctcgtagacgcctcagtccagtctctgcctccgtgttcactg

SEQ ID NO: 412 - DNA, 119 bp, Bos taurus
  misc_feature (60)..(60): C OR T
agctcctggtgccgcacccttgggcccgatctcgtagacgcctcagtccagtctctgccnccgtgttcactggcattctccccatgtcccctctgtgtccccgttttctctcacaagga

SEQ ID NO: 413 - DNA, 119 bp, Bos taurus
  misc_feature (60)..(60): G OR T
ccgcacccttgggcccgatctcgtagacgcctcagtccagtctctgcctccgtgttcacnggcattctccccatgtcccctctgtgtccccgttttctctcacaaggacaccggacata

SEQ ID NO: 414 - DNA, 119 bp, Bos taurus
  misc_feature (60)..(60): A OR G
gacctagattccaaacaagattccatcctgaagttcctggtggacgtgagttctggagcnacgcccttcaaccccatcacagcttgcggttcatcgcaaaacacggaacctgggattta

SEQ ID NO: 415 - DNA, 119 bp, Bos taurus
  misc_feature (60)..(60): A OR G
agcttgcggttcatcgcaaaacacggaacctgggatttatcgtaaaacccaggttcttcntgaaacactgagcttcgaggcttgttgcaagaattaaaggtgctaatacagatcagggc

SEQ ID NO: 416 - DNA, 119 bp, Bos taurus
  misc_feature (60)..(60): G OR A
aacagtctctccgggaaggaaaccagaggccagagagcaagccagagctagtctaggagntccctgagcctccacccaagatgccgaccaggccagcgggccccctggaaagaccctac

SEQ ID NO: 417 - DNA, 119 bp, Bos taurus
  misc_feature (60)..(60): C OR A
tccctgagcctccacccaagatgccgaccaggccagcgggccccctggaaagaccctacngtctagggggggaacaggagccgacccgccaggcccccgctatcaggagacaccccaac

SEQ ID NO: 418 - DNA, 119 bp, Bos taurus
  misc_feature (60)..(60): G OR C
agggggggaacaggagccgacccgccaggcccccgctatcaggagacaccccaaccttgntcctgttcccctaccccagtacgcccacccgacccctgagatgagtggtttacttgctt

SEQ ID NO: 419 - DNA, 119 bp, Bos taurus
  misc_feature (60)..(60): A OR C
caggcccccgctatcaggagacaccccaaccttgctcctgttcccctaccccagtacgcncacccgacccctgagatgagtggtttacttgcttagaatgtcaattgaaggcttttgta

SEQ ID NO: 420 - DNA, 119 bp, Bos taurus
  misc_feature (60)..(60): C OR T
caccccaaccttgctcctgttcccctaccccagtacgcccacccgacccctgagatgagnggtttacttgcttagaatgtcaattgaaggcttttgtaccccctttgccagtggcacag

SEQ ID NO: 421 - DNA, 119 bp, Bos taurus
  misc_feature (60)..(60): A OR G
accccaaccttgctcctgttcccctaccccagtacgcccacccgacccctgagatgagtngtttacttgcttagaatgtcaattgaaggcttttgtaccccctttgccagtggcacagg

SEQ ID NO: 422 - DNA, 119 bp, Bos taurus
  misc_feature (60)..(60): G OR C
accttgctcctgttcccctaccccagtacgcccacccgacccctgagatgagtggtttanttgcttagaatgtcaattgaaggcttttgtaccccctttgccagtggcacagggcaccc

SEQ ID NO: 423 - DNA, 119 bp, Bos taurus
  misc_feature (60)..(60): G OR A
ccctccctgctcgggccccctccatactcagcgacacacccagcaccagcattcccaccnctcctgaggtctgaaggcagctcgctgtggtctgagcggtgcggagggaagtgccctgg

SEQ ID NO: 424 - DNA, 119 bp, Bos taurus
  misc_feature (60)..(60): C OR G
gagggaagtgccctgggagatttaaaatgtgagaggtgggaggtgggaggttgggtcctntaggccttcccatcccacgtgcctgcacggagccctagtgctactcagtcatgcccccg

SEQ ID NO: 425 - DNA, 119 bp, Bos taurus
  misc_feature (60)..(60): T OR C
gtgagaggtgggaggtgggaggttgggtcctgtaggccttcccatcccacgtgcctgcanggagccctagtgctactcagtcatgcccccgcagcaggggtcaggtcactttcccatcc

SEQ ID NO: 426 - DNA, 119 bp, Bos taurus
  misc_feature (60)..(60): C OR A
aggccttcccatcccacgtgcctgcacggagccctagtgctactcagtcatgcccccgcngcaggggtcaggtcactttcccatcctgggggttattatgactgttgtcattgttgttg

SEQ ID NO: 427 - DNA, 119 bp, Bos taurus
  misc_feature (60)..(60): G OR C
gggcagcgggtgcttgcagagccctcgatactgaccaggttcccccctcggagctcgacntgaaccccatgtcaccctcgccccagcctgcagagggtggggtgactgcagagatccct

SEQ ID NO: 428 - DNA, 119 bp, Bos taurus
  misc_feature (60)..(60): G OR C
atactgaccaggttcccccctcggagctcgacctgaaccccatgtcaccctcgccccagnctgcagagggtggggtgactgcagagatccctttacccaaggccacagtcacatggttt

SEQ ID NO: 429 - DNA, 119 bp, Bos taurus
  misc_feature (60)..(60): T OR C
aagtgttcctggcgctggcagccagcctggacccagagcctggacaccccctgcgccccnacttctggggcgtaccaggaaccgtccaggcccagagggggccttcctgcttggcctcg

SEQ ID NO: 430 - DNA, 119 bp, Bos taurus
  misc_feature (60)..(60): C OR G
cgctggcagccagcctggacccagagcctggacaccccctgcgcccccacttctggggcntaccaggaaccgtccaggcccagagggggccttcctgcttggcctcgaatggaagaagg

SEQ ID NO: 431 - DNA, 119 bp, Bos taurus
  misc_feature (60)..(60): C OR G
gaatggaagaaggcctcctattgtcctcgtagaggaagcaaccccagggcccaaggatangccaggggggattcggggaaccgcgtggctgggggcccggcccgggctggctggctggc

SEQ ID NO: 432 - DNA, 119 bp, Bos taurus
  misc_feature (60)..(60): T OR C
tataaggccccgagcccactgtctcagccctccactccctgcagagctcagaagcgtgancccagctgcagccatgaagtgcctcctgcttgccctggccctcacttgtggcgcccagg

SEQ ID NO: 433 - DNA, 119 bp, Bos taurus
  misc_feature (60)..(60): G OR A
aggccccgagcccactgtctcagccctccactccctgcagagctcagaagcgtgaccccngctgcagccatgaagtgcctcctgcttgccctggccctcacttgtggcgcccaggccct

SEQ ID NO: 434 - DNA, 119 bp, Bos taurus
  misc_feature (60)..(60): C OR T
ctcagaagcgtgaccccagctgcagccatgaagtgcctcctgcttgccctggccctcacntgtggcgcccaggccctcattgtcacccagaccatgaagggcctggatatccagaaggt

SEQ ID NO: 435 - DNA, 119 bp, Bos taurus
  misc_feature (60)..(60): C OR T
gcagccatgaagtgcctcctgcttgccctggccctcacttgtggcgcccaggccctcatngtcacccagaccatgaagggcctggatatccagaaggttcgagggtgcccgggtgggtg

SEQ ID NO: 436 - DNA, 119 bp, Bos taurus
  misc_feature (60)..(60): G OR C
caggccctcattgtcacccagaccatgaagggcctggatatccagaaggttcgagggtgnccgggtgggtggtgagttgcagggcaggcaggggagctgggcctcagagaccaagggag

SEQ ID NO: 437 - DNA, 119 bp, Bos taurus
  misc_feature (60)..(60): A OR G
gggcagcttcaaccaggcgtttagtgtcttgcattctggaggctggaagcctgcaatccnggcatcggcccagctggcttctcctgcggccactctccggggagcagacagccatcttc

SEQ ID NO: 438 - DNA, 119 bp, Bos taurus
  misc_feature (60)..(60): G OR A
cctcttcctgtgaggtcaccaggcctgctggatccacgcccgcccacacagcctcacgtnacctttgtcatctctttaaaggccgtgtctccagtcctgtgttgaggttctgggggtta

SEQ ID NO: 439 - DNA, 119 bp, Bos taurus
  misc_feature (60)..(60): C OR T
agagtcactctgcccctcaaattttccccacctccagctatgtctccccaagatccaaangttgccacgtgtgcgggggctcatctgggtccctctttgggctcagagtgagtctgggg

SEQ ID NO: 440 - DNA, 119 bp, Bos taurus
  misc_feature (60)..(60): G OR C
cagggctgcccaggccagggtgggacagagagcccactgtggggctgggggccccttccngcccctggagtgcagctcaaggtccctccccaggtggcggggacttggtactccttggc

SEQ ID NO: 441 - DNA, 119 bp, Bos taurus
  misc_feature (60)..(60): C OR CA
gaagcccacccctgagggcgacctggagatcctgctgcagaaatggtgggcgtccccccnaaaaaaagcatggaacccccactccccagggatatggacccccccggggtggggtgcag

SEQ ID NO: 442 - DNA, 119 bp, Bos taurus
  misc_feature (60)..(60): T OR C
gggcttggagtttcctggtacccctggaggtccacccaaggctgcttatccagggctttntctttctttttttcccccaacttttattaatttgatgcttcagaacatcatcaaacaaa

SEQ ID NO: 443 - DNA, 119 bp, Bos taurus
  misc_feature (60)..(60): G OR A
agatggggacctgaaccccaggactgccttttggggtgcctgtggtcaaggctctccccnaccttttctccctggctccatctgacttctcctggcccatccacccggtcacctgtggc

SEQ ID NO: 444 - DNA, 119 bp, Bos taurus
  misc_feature (60)..(60): G OR A
cttctcctggcccatccacccggtcacctgtggccccagaggtgacagtgagtgcagccnaggccggttggccagccggccccctatgcccacgccacccgcctccagcccctcctggg

SEQ ID NO: 445 - DNA, 119 bp, Bos taurus
  misc_feature (60)..(60): T OR C
cacccggtcacctgtggccccagaggtgacagtgagtgcagccaaggccggttggccagncggccccctatgcccacgccacccgcctccagcccctcctggggccgccttctgcccct

SEQ ID NO: 446 - DNA, 119 bp, Bos taurus
  misc_feature (60)..(60): A OR G
tcctgatgaaaatggtccatgcccgtggctcagaaagcagctgtctttcagggagaacgntgagtgtgctcagaagaagatcattgcagaaaaaaccaagatccctgcggtgttcaaga

SEQ ID NO: 447 - DNA, 119 bp, Bos taurus
  misc_feature (60)..(60): A OR G
gggacgcccaccaccccccagggactgtgggcaggtgcagggggctggcgtcaggccccnagatgctaaggggctggtggtgatgaagacactgccgtgccacctgcttccctggcctc

SEQ ID NO: 448 - DNA, 119 bp, Bos taurus
  misc_feature (60)..(60): G OR A
ggacgcccaccaccccccagggactgtgggcaggtgcagggggctggcgtcaggccccgngatgctaaggggctggtggtgatgaagacactgccgtgccacctgcttccctggcctcc

SEQ ID NO: 449 - DNA, 119 bp, Bos taurus
  misc_feature (60)..(60): C OR T
gatgaagacactgccgtgccacctgcttccctggcctccctgccacctgcccggggcctnggggccggtggccgtgggcaggtcccggctgggcaggtctgacaccccagggtgacacc

SEQ ID NO: 450 - DNA, 119 bp, Bos taurus
  misc_feature (60)..(60): A OR G
atgaagacactgccgtgccacctgcttccctggcctccctgccacctgcccggggccttngggccggtggccgtgggcaggtcccggctgggcaggtctgacaccccagggtgacaccc

SEQ ID NO: 451 - DNA, 119 bp, Bos taurus
  misc_feature (60)..(60): T OR C
gcaggtctgacaccccagggtgacacccgagctctctttgctgagggtggggtggtgctnggggccctcaggctgagctcaggaggtccctgtgcccacccaggggtaaccgagagccg

SEQ ID NO: 452 - DNA, 119 bp, Bos taurus
  misc_feature (60)..(60): T OR TA
tggggtggtgctcggggccctcaggctgagctcaggaggtccctgtgcccacccaggggnaaccgagagccgctgcccgctccaggggtccaggtgccccacgatcccagcccacccca

SEQ ID NO: 453 - DNA, 119 bp, Bos taurus
  misc_feature (60)..(60): A OR G
cacatctgcccttggcctcttcaggactcactctgactggaggccctgcactgactgatnccagggtgcccagcccagggtctcctgtgccatccggctgcacggggtttggatgctgg

SEQ ID NO: 454 - DNA, 119 bp, Bos taurus
  misc_feature (60)..(60): G OR A
ggggtttggatgctggtcctgcccccaagctgcccagacactgcagggcagctggggccncccgcaggcctcggtcagggagagccccagctgcccccgctcagcgctgccccccaaca

SEQ ID NO: 455 - DNA, 119 bp, Bos taurus
  misc_feature (60)..(60): T OR C
cactgcagggcagctggggccacccgcaggcctcggtcagggagagccccagctgccccngctcagcgctgccccccaacaattccccagtcctcaggacgcatccctcttcccttgct

SEQ ID NO: 456 - DNA, 119 bp, Bos taurus
  misc_feature (60)..(60): G OR A
gtggagggtccctggcggatccagagttgggcttccagagtgagggcttcctgggccccntgtgcctggcagtggcagcagggaaggggccacaccattttggggctgggggatgccag

SEQ ID NO: 457 - DNA, 119 bp, Bos taurus
  misc_feature (60)..(60): A OR G
tccctggcggatccagagttgggcttccagagtgagggcttcctgggccccatgtgcctngcagtggcagcagggaaggggccacaccattttggggctgggggatgccagagggcgct

SEQ ID NO: 458 - DNA, 119 bp, Bos taurus
  misc_feature (60)..(60): C OR T
gagggcttcctgggccccatgtgcctggcagtggcagcagggaaggggccacaccatttnggggctgggggatgccagagggcgctccccaccccgtcctcaccaagtggtgaccccgg

SEQ ID NO: 459 - DNA, 119 bp, Bos taurus
  misc_feature (60)..(60): A OR G
agggcttcctgggccccatgtgcctggcagtggcagcagggaaggggccacaccattttngggctgggggatgccagagggcgctccccaccccgtcctcaccaagtggtgaccccggg

SEQ ID NO: 460 - DNA, 119 bp, Bos taurus
  misc_feature (60)..(60): C OR T
ctccccaccccgtcctcaccaagtggtgaccccgggggagccccgctggttgtggggggngctgggggctgaccagaaacccccctcctgctggaactcactttcctcctgtcttgatc

SEQ ID NO: 461 - DNA, 119 bp, Bos taurus
  misc_feature (60)..(60): A OR T
gggggctgaccagaaacccccctcctgctggaactcactttcctcctgtcttgatctctnccagccttgaacgagaacaaagtccttgtgctggacaccgactacaaaaagtacctgct

SEQ ID NO: 462 - DNA, 119 bp, Bos taurus
  misc_feature (60)..(60): C OR T
gaaacccccctcctgctggaactcactttcctcctgtcttgatctctaccagccttgaangagaacaaagtccttgtgctggacaccgactacaaaaagtacctgctcttctgcatgga

SEQ ID NO: 463 - DNA, 119 bp, Bos taurus
  misc_feature (60)..(60): T OR C
ctacaaaaagtacctgctcttctgcatggagaacagtgctgagcccgagcaaagcctggnctgccagtgcctgggtgggtgccaaccctggctgcccagggagaccagctgtgtggtcc

SEQ ID NO: 464 - DNA, 119 bp, Bos taurus
  misc_feature (60)..(60): T OR C
gggtccccgagtcccgccaggagagggtggtcatataccgggagccggtgtcctgggggnctgtgggtgactggggacgggggccagacacacaggctgggagacggggggctgcagcg

SEQ ID NO: 465 - DNA, 119 bp, Bos taurus
  misc_feature (60)..(60): T OR G
tgccactgggcattttcagggcccatgtgccaggagggcgtgggcatcggcgagtggagnctcctggccgtgtcagctggcccagggggaggaggggacccagacagccagaggtgggg

SEQ ID NO: 466 - DNA, 119 bp, Bos taurus
  misc_feature (60)..(60): A OR G
ccccctccccaccccctccccagctccctctgtcctggggtgtccagtcccatcctgacnctcccccgccacggctctccctcccccacagagcagtgccacatctaggtgagcccctg

SEQ ID NO: 467 - DNA, 119 bp, Bos taurus
  misc_feature (60)..(60): C OR T
ccctccccaccccctccccagctccctctgtcctggggtgtccagtcccatcctgacgcncccccgccacggctctccctcccccacagagcagtgccacatctaggtgagcccctgcc

SEQ ID NO: 468 - DNA, 119 bp, Bos taurus
  misc_feature (60)..(60): G OR A
ccaaggaggggccgtcctgaatccccagccacggacaggctggcaagggtctggcaggtnccccaggaatcacaggggagccccatgtccatttcagagcccgggagccttggcccctc

SEQ ID NO: 469 - DNA, 119 bp, Bos taurus
  misc_feature (60)..(60): G OR A
gcatcaccgacaaaatggacatgggtttgggtggactccaggagttggtgatggacaganaggcctggcatgctgcggtccatggggtcacaaagagtcggacacgactgagcgactga

SEQ ID NO: 470 - DNA, 119 bp, Bos taurus
  misc_feature (60)..(60): G OR A
actcatatttatatatatacgtgaatgctcagtcacactcagtcatatctgactctgtgncccatggactgtagccctccaggctccttctgtccacagaattctccaggcaagaatac

SEQ ID NO: 471 - DNA, 119 bp, Bos taurus
  misc_feature (60)..(60): A OR G
atcccctagcctggaaggcagattcttaaccactggaccaccagggaagtcccctgcctncctgcatcttaacctcagttcagtcgctcagttgtgtccgactctttgcgacccgatga

SEQ ID NO: 472 - DNA, 119 bp, Bos taurus
  misc_feature (60)..(60): G OR A
gacacagcattgctgtccggcgaatggagccaccatctggatccttcaagtgaccctggncggggcagggctggcactggccgtccggcctgcacttccgtcgggctccggactccagg

SEQ ID NO: 473 - DNA, 119 bp, Bos taurus
  misc_feature (60)..(60): C OR A
acaagaagtgtgggagaaatgaccaagatatcttgagggaaaaaaataaggttatatatntaactctgcacattatcttaaaattaattctgaatgaaaagaaatcttgaaagctctat

SEQ ID NO: 474 - DNA, 119 bp, Bos taurus
  misc_feature (60)..(60): G OR A
tataccttaaaaactaaaaaaataaaataaaataggctttgatgtcccaagcaatcaaanaaaaagattctatgtttgcttatcaaattagcaaaattttttaaaaatttaactcagca

SEQ ID NO: 475 - DNA, 119 bp, Bos taurus
  misc_feature (60)..(60): G OR A
ttttcggaagggcatcaccaaacactatccacatgggaggcgccaggtcgacactgcagnattcccagggaggaaggcaggaggtcgggcctgtatttcagctgcaatggttacagaag

SEQ ID NO: 476 - DNA, 119 bp, Bos taurus
  misc_feature (60)..(60): C OR T
tgttttgtagaataatattagaaaccaaggtctggggctgggagtgctcgctgctcctgngggctcctcgtttctaggtcctctcagctgggcaagcagggaaggcatgtgtgtagttg

SEQ ID NO: 477 - DNA, 119 bp, Bos taurus
  misc_feature (60)..(60): T OR G
ctgatgctggaaaagattgatggcaggatgataagggggcaaccgaggataagatgcttngatggcatcaccaactcaatggacatgaatttaagcagattccaggagttggtgaagga

SEQ ID NO: 478 - DNA, 119 bp, Bos taurus
  misc_feature (60)..(60): T OR TA
aagggggcaaccgaggataagatgcttggatggcatcaccaactcaatggacatgaattnaagcagattccaggagttggtgaaggacagggaagcctggcgtgctgcagtccatgggc

SEQ ID NO: 479 - DNA, 119 bp, Bos taurus
  misc_feature (60)..(60): G OR A
ccatgggctgagaaacaacaaagcacagactccactggtttccaaggtgacttctctcancaccttcctcagccctcagggagcttacttctggcatgtttgatacagttcgatgactt

SEQ ID NO: 480 - DNA, 119 bp, Bos taurus
  misc_feature (60)..(60): G OR A
ctttactgtctctgcagttttgccttttccagaatttcacgtggctggaatcatacagtnggtagtcttttcagattggcttctggcttctttcccttaccaatgtgtactgatacttc

SEQ ID NO: 481 - DNA, 119 bp, Bos taurus
  misc_feature (60)..(60): T OR C
gtgtccgactctgtgtgaccccatagactatacagtccatggaattctccaggccagaanactggagtggatagccattcccttctccaggggatcttcccaacccagggatcgaactc

SEQ ID NO: 482 - DNA, 119 bp, Bos taurus
  misc_feature (60)..(60): A OR AG
taggctatcccagggacttccctggtggcccagtggctaggactctgcggttccagttcngggggcctgggtttgaccccaggtcagggaactagatcccacttgccaaaactaagggt

SEQ ID NO: 483 - DNA, 119 bp, Bos taurus
  misc_feature (60)..(60): A OR C
caaaaatatactttccagtttcattgctgatagaactgatctcggaaacagagggtctgnatcaaggtcacctagtaggttacaggaccagatgtagtttccatctctgtctgcaccat

SEQ ID NO: 484 - DNA, 119 bp, Bos taurus
  misc_feature (60)..(60): G OR A
atactttccagtttcattgctgatagaactgatctcggaaacagagggtctgaatcaagntcacctagtaggttacaggaccagatgtagtttccatctctgtctgcaccattatcatt

SEQ ID NO: 485 - DNA, 119 bp, Bos taurus
  misc_feature (60)..(60): G OR A
tttcattgctgatagaactgatctcggaaacagagggtctgaatcaaggtcacctagtangttacaggaccagatgtagtttccatctctgtctgcaccattatcattaggtgattctg

SEQ ID NO: 486 - DNA, 119 bp, Bos taurus
  misc_feature (60)..(60): C OR T
ttgctgatagaactgatctcggaaacagagggtctgaatcaaggtcacctagtaggttanaggaccagatgtagtttccatctctgtctgcaccattatcattaggtgattctgtgttg

SEQ ID NO: 487 - DNA, 119 bp, Bos taurus
  misc_feature (60)..(60): G OR A
ctgatagaactgatctcggaaacagagggtctgaatcaaggtcacctagtaggttacagnaccagatgtagtttccatctctgtctgcaccattatcattaggtgattctgtgttgcag

SEQ ID NO: 488 - DNA, 119 bp, Bos taurus
  misc_feature (60)..(60): T OR C
atctcggaaacagagggtctgaatcaaggtcacctagtaggttacaggaccagatgtagnttccatctctgtctgcaccattatcattaggtgattctgtgttgcagagccaagtagca

SEQ ID NO: 489 - DNA, 119 bp, Bos taurus
  misc_feature (60)..(60): C OR T
agggtctgaatcaaggtcacctagtaggttacaggaccagatgtagtttccatctctgtntgcaccattatcattaggtgattctgtgttgcagagccaagtagcactactatgattac

SEQ ID NO: 490 - DNA, 119 bp, Bos taurus
  misc_feature (60)..(60): C OR A
gtctgaatcaaggtcacctagtaggttacaggaccagatgtagtttccatctctgtctgnaccattatcattaggtgattctgtgttgcagagccaagtagcactactatgattacata

SEQ ID NO: 491 - DNA, 119 bp, Bos taurus
  misc_feature (60)..(60): C OR T
ctgaatcaaggtcacctagtaggttacaggaccagatgtagtttccatctctgtctgcancattatcattaggtgattctgtgttgcagagccaagtagcactactatgattacatatc

SEQ ID NO: 492 - DNA, 119 bp, Bos taurus
  misc_feature (60)..(60): T OR A
aatcaaggtcacctagtaggttacaggaccagatgtagtttccatctctgtctgcaccantatcattaggtgattctgtgttgcagagccaagtagcactactatgattacatatcttt

SEQ ID NO: 493 - DNA, 119 bp, Bos taurus
  misc_feature (60)..(60): C OR A
aaggtcacctagtaggttacaggaccagatgtagtttccatctctgtctgcaccattatnattaggtgattctgtgttgcagagccaagtagcactactatgattacatatcttttcct

SEQ ID NO: 494 - DNA, 119 bp, Bos taurus
  misc_feature (60)..(60): T OR A
gtaggttacaggaccagatgtagtttccatctctgtctgcaccattatcattaggtgatnctgtgttgcagagccaagtagcactactatgattacatatcttttcctgtagccttttg

SEQ ID NO: 495 - DNA, 119 bp, Bos taurus
  misc_feature (60)..(60): C OR T
gtaccatctccctctgggacggtcggtgctgccaccatcccccaggagtgacaactgagnccagtcaaggacactctcagatacgcctcctcacagtcaggataccctatgctactcgc

SEQ ID NO: 496 - DNA, 119 bp, Bos taurus
  misc_feature (60)..(60): A OR C
cctgagatagttacttaaattctctataccacatgtttcctcattctgaaaacagagatnatgatagtacttttacaaatgctattgctgtagagattaaatgtattaatccaggtaaa

SEQ ID NO: 497 - DNA, 119 bp, Bos taurus
  misc_feature (60)..(60): A OR G
caggcacaatagcaaggcccctcttgtgttattttatgttattttcccctcaacctcttnttgtactgttttttctggatattattttatgttattttcccctaaacctcttattattc

SEQ ID NO: 498 - DNA, 119 bp, Bos taurus
  misc_feature (60)..(60): T OR C
tttcagccattggccactacacgtctcctttttctactaatatctgatctaaagcaattncattttctgaaaaattcaatagttataagaggataccttgaaaccataattacagctac

SEQ ID NO: 499 - DNA, 119 bp, Bos taurus
  misc_feature (60)..(60): A OR T
caattaaaataaataaattaaaaatatatataaaatataaataaaaatttttcaggattnaaaaaaaaggtcacaacactggaatttcaagttaccaaaatctgctgctaagctaagtc

SEQ ID NO: 500 - DNA, 119 bp, Bos taurus
  misc_feature (60)..(60): C OR T
ctcctgcctgtgcctccatgtggggcccctttcatttattttccacctatcctcagagcnttgttcaaattccgcctcctccaggaagccttccgggtcaatccagctggaaatgcgct

SEQ ID NO: 501 - DNA, 119 bp, Bos taurus
  misc_feature (60)..(60): G OR A
cccaagcttattcttctatccatgagactctcctgcaagcaccctcctctgtaggcttcngcttccccacctgcagtaatcagaggggccatcagtgagggcctcctgagggccaggca

SEQ ID NO: 502 - DNA, 119 bp, Bos taurus
  misc_feature (60)..(60): C OR G
acaattataaacaatgcatggtgcttgtggaaacccagactcaaggcatttatgaaaacnaaactcccccgtggagggcaggaagggcagtgggatagaggagtagaggaatgcaatgg

SEQ ID NO: 503 - DNA, 119 bp, Bos taurus
  misc_feature (60)..(60): G OR A
cctgccacgcagaagaggcaggttcaatccctgggtcaggaagagaaggaaatggcagcncactccagtatcccatgacagaggaacctggtaggtatagtctatggggttgcaaaaga

SEQ ID NO: 504 - DNA, 119 bp, Bos taurus
  misc_feature (60)..(60): T OR C
ataggtatgatgcagttcataataaaatgtctaaaaagagaccaaaataaataaagaagngatgagaggactttctggtagtccaacgattaagaatccacccaccagtgcagggaatg

SEQ ID NO: 505 - DNA, 119 bp, Bos taurus
  misc_feature (60)..(60): G OR GA
aatgagtagtttctagtgacatgaaaaaacgctgatagtgacatgaaaaaatacagatanaaaaaaaaagataatagtgaaaaaaagctggatgcagaacacattaacagcatggtctg

SEQ ID NO: 506 - DNA, 119 bp, Bos taurus
  misc_feature (60)..(60): G OR A
aaactcccagccataccaggtatttctctggtgtatagataggctttctaaagcaccatnagcatttcagactcctgtttacccaacaaggatcatatacctttaaattgcttcccctc

SEQ ID NO: 507 - DNA, 119 bp, Bos taurus
  misc_feature (60)..(60): C OR T
tcccattctgttgttttcctctatttctttgcattgctgaggaaggctttcttatctctncttgctattctttggaactctgcattcaaatgagtatatctttcctttactcctttgct

SEQ ID NO: 508 - DNA, 119 bp, Bos taurus
  misc_feature (60)..(60): G OR A
tttcatgcactggaggaggaaatggcaacccactccagtgttcttgcctggagaatcccntggacagaggagcctggtgggctgccgtctatggggtcgcacagagtcggacacgactg

SEQ ID NO: 509 - DNA, 119 bp, Bos taurus
  misc_feature (60)..(60): T OR G
ttcatgcactggaggaggaaatggcaacccactccagtgttcttgcctggagaatcccgnggacagaggagcctggtgggctgccgtctatggggtcgcacagagtcggacacgactga

SEQ ID NO: 510 - DNA, 119 bp, Bos taurus
  misc_feature (60)..(60): G OR A
acattagttttaggcttctttgtaattttttcttgtttgttgatggtttgacaaaaggcnaagtttttaaatactcggttttgaaaggtgtttttttattttttatttaagtgtatttg

SEQ ID NO: 511 - DNA, 119 bp, Bos taurus
  misc_feature (60)..(60): C OR T
tctttttgtttggagcaacctcatgaagttgttttgttttttgttttttaacagtcaaantgtatggctcattgtagaaaatacagaaacatataaagaatttactagttcatcatccc

SEQ ID NO: 512 - DNA, 119 bp, Bos taurus
  misc_feature (60)..(60): G OR A
atcacacatatattcctgataggaatgtaagataggacaatcactctggaaaatacttcntcagtctcctctaaaactaaatatatatgtaccgtgtgtatgtgctcagaccctgagtc

SEQ ID NO: 513 - DNA, 119 bp, Bos taurus
  misc_feature (60)..(60): C OR T
gtcacacagggattggtgaactacagaccctcagatatggcagggagccacgagctcgtngtctttgactcccatccccaactctatacctgaatcctgccccatccctcagccctctc

SEQ ID NO: 514 - DNA, 119 bp, Bos taurus
  misc_feature (60)..(60): C OR T
tgtctctgcttttgaatatgctatctagcttggtcataaccttccttccaaggagtaagngtattttcatttcatggctgcagtcaccatctgcagtgattttggagcccaaaaaaata

SEQ ID NO: 515 - DNA, 119 bp, Bos taurus
  misc_feature (60)..(60): A OR G
gagtaagcgtattttcatttcatggctgcagtcaccatctgcagtgattttggagcccanaaaaataaagtctgacactgtttccactatttccccatctatttcccatgaagtgatgg

SEQ ID NO: 516 - DNA, 119 bp, Bos taurus
  misc_feature (60)..(60): GAGATGAGTGCAATTGTGCA OR GAGATGAGTGCAATTGTGCG
ggttcacatattgctgaagcctggcttggagaattttgagcattactttactagcgtgtnagatgagtgcaattgtgcagtagtttgagcattctttggcattgcctttctttgggatt

SEQ ID NO: 517 - DNA, 119 bp, Bos taurus
  misc_feature (60)..(60): T OR C
ggccaggggtccccaaatggaggagacagactacaagtgtcagacattttaaactatctnttaagtggcaggaggaaacaaacaagtgttagatttcttcccctcctctatacaaattt

SEQ ID NO: 518 - DNA, 119 bp, Bos taurus
  misc_feature (60)..(60): C OR A
gcctggagaatcccagggaccggggagcctggtgggctgccatctatggggtcgcacagngtcggacacgactgaatcgacttagcagcagcagcagtagctacataacatccaactaa

SEQ ID NO: 519 - DNA, 119 bp, Bos taurus
  misc_feature (60)..(60): T OR G
tctcctccggaggccaagaatcctggcgtctttcatggctcagcaacaacctttcaatanaagtactattatcagccccattttataaatattgaggcttagagagcctaaatgagttg

SEQ ID NO: 520 - DNA, 119 bp, Bos taurus
  misc_feature (60)..(60): T OR G
acccaaatgaagggatacaggggctggcatgggaaatagtcaagacaatagggccaggcngcaaagcacggagtgcctagaaaaagcgtagttagacccccggctcagaaggcgcacag

SEQ ID NO: 521 - DNA, 119 bp, Bos taurus
  misc_feature (60)..(60): G OR A
ccccggctcagaaggcgcacagaacccaaaatccagcgcccactgcgccgacagtggacnccaagacctagagacacactagggctgctcaagggcgcacagcgcagcggcgccggtcc

SEQ ID NO: 522 - DNA, 119 bp, Bos taurus
  misc_feature (60)..(60): C OR A
cgcccactgcgccgacagtggacgccaagacctagagacacactagggctgctcaagggngcacagcgcagcggcgccggtcccagaggcagcttctccaaccccaccagatcgatcct

SEQ ID NO: 523 - DNA, 119 bp, Bos taurus
  misc_feature (60)..(60): T OR G
cactgccccgttccggaagcccttaagcatggccagtgctgcgtggtagcggcgcttgcncaatagtgcgttgatcgcaaagagcagagcccgcagctgcggtggggcgaccataatgc

SEQ ID NO: 524 - DNA, 119 bp, Bos taurus
  misc_feature (60)..(60): G OR C
aggatgtgtctgctgtcactcatacgagcagggccgatcacagagctcactcaactcacnaggggaatgggacgcaagcactctcccgaagagcggtctgattggaagagagggaaagc

SEQ ID NO: 525 - DNA, 119 bp, Bos taurus
  misc_feature (60)..(60): T OR C
aggagtacatcaaagctgtatactgtcaccctgctgatttaacttatatgcagagtacancatgagaaacgctgggctggaagaagcacaagctggaatcaagattgccagaagaaata

SEQ ID NO: 526 - DNA, 119 bp, Bos taurus
  misc_feature (60)..(60): C OR T
gtcttttctggcaatttgatctctggttcctctgccttttctaaaaccagcttgaacatngggaagttcacggttcatgtactgttgaagccaagattggagaattttgagcattactt

SEQ ID NO: 527 - DNA, 119 bp, Bos taurus
  misc_feature (60)..(60): C OR T
ttttctaatgagtcagctcttcgcaccaagtggccatagtattggaacttcagcttcagnatcagtccttccaatgaatattcagggttgatttcctttaagatggactcatttgatct

SEQ ID NO: 528 - DNA, 119 bp, Bos taurus
  misc_feature (60)..(60): C OR T
tggatggggaagctgatgtgttcagctgggacccacagcctgactttggggtttcatagncatagtccagttcttttgactcgtacttttattcttttttttaaaaattactatatttt

SEQ ID NO: 529 - DNA, 119 bp, Bos taurus
  misc_feature (60)..(60): C OR T
aatagatgccacctctcaatgggaaaaactgcaggtcacactggaaagggcctgggtacnagaagaagtaaagagtcgaggccattcagtcagtgtacaggataccttgcttcattgca

SEQ ID NO: 530 - DNA, 119 bp, Bos taurus
  misc_feature (60)..(60): G OR A
aaggcctgacctcggggttagtgatgctctatgaacgtgcctctgcaagttctctgctcnctcagtactaaccatgacaccccttgtcccaagaacatgcaaggagcccctctgggcct

SEQ ID NO: 531 - DNA, 119 bp, Bos taurus
  misc_feature (60)..(60): G OR A
agctcaagtcccacatgacccagcacaagaatgagcaggtaggtgagggggcgggagccngggtggggcacaaccacccaagtcatgagccttggctgatgctggggtcttgaacacgg

SEQ ID NO: 532 - DNA, 119 bp, Bos taurus
  misc_feature (60)..(60): G OR A
ggactgcaggtgaggaggctggggcaatattggggttctcgtagctaaatctgtcctagnttggcatatgggagacgggcccagagaggggaaggcccacggtcaagggtcactcaaag

SEQ ID NO: 533 - DNA, 119 bp, Bos taurus
  misc_feature (60)..(60): C OR T
ttttcatgttcctggcccactttactcattcatcttatttatagactctgggtatatgantttgaccctggtggctcagatgtaaagaatctgcctgcaatgcaggagacctaggtttg

SEQ ID NO: 534 - DNA, 119 bp, Bos taurus
  misc_feature (60)..(60): G OR A
acttccctggtggtcctgtggttaaaacgccacgcttccactgcagtgagcatagtctgnatccctggttgagaactaagatcccacatgccgatgatgtggccaaaagaaggggatgg

SEQ ID NO: 535 - DNA, 119 bp, Bos taurus
  misc_feature (60)..(60): G OR T
tgcagctgaagaagctgatggtcagggacaggaggatacttttaccagcaagcagaggtngaatgtgaaatcaggtcacctgatcccaaacttagagcttttttttactagaccatatc

SEQ ID NO: 536 - DNA, 119 bp, Bos taurus
  misc_feature (60)..(60): C OR T
cttttatcagctacttgtacgtgtgtgtgtcatctgcactgaactgtgtgcccttgtagngtctcatccatctttctacctcttagtgtgcctgagactatagccgggaaaccaaggaa

SEQ ID NO: 537 - DNA, 119 bp, Bos taurus
  misc_feature (60)..(60): A OR T
aaaggagatcagtcctgggtgttcattggagggactgatgttgaagctgaaatcccaatnctttggccacctgatgcggagagctgattcatttgaaaagaccctgatgctgggaaaga

SEQ ID NO: 538 - DNA, 119 bp, Bos taurus
  misc_feature (60)..(60): C OR G
tctgagcgcctactgcacactgccaggaaagatgggcttgtgtcagtgcggggagcctgncttctccctgtccctggccatgcagaccctccttgctacctctcagctctcagaacagc

SEQ ID NO: 539 - DNA, 119 bp, Bos taurus
  misc_feature (60)..(60): G OR T
gagccgtattcatcgtgtccctgaggcctgggcttcagatgaacagaccctctgcacaangggaagttgaggcaggacagagtcatgccacttaccctaaaaaatacctgctactgaaa

SEQ ID NO: 540 - DNA, 119 bp, Bos taurus
  misc_feature (60)..(60): T OR C
agagtccacatgccaagggacaaccatcacatgccaagtatcacaaccattgaaccctgngggctctagagcctgtgctctgcaacaagagaagacactgcaaggagaagcatgtgccc

SEQ ID NO: 541 - DNA, 119 bp, Bos taurus
  misc_feature (60)..(60): G OR A
ccatgtcttgactgtttgcagccctgtggactgtagcatgccaggctcctctgtccactntctcccaaagtttgctcagattcatgtccattgagtcaatgatgctatctaaccatctc

SEQ ID NO: 542 - DNA, 119 bp, Bos taurus
  misc_feature (60)..(60): G OR A
tgcagtttgctaacacctaagctacagtgtgaagaaagagcttaagtcatggagttattngaaccagttgtaggtcgtacactgtaattttaacaatgctactgcaagagctatctggt

SEQ ID NO: 543 - DNA, 119 bp, Bos taurus
  misc_feature (60)..(60): T OR TA
accaaaacataaaacagaagcaatattattacaaataaaggctttaaaaatggttcacanaaaaaaaaaatcttaagaaaataaagggaatatattggcttgagtgtgtgctgagtcac

SEQ ID NO: 544 - DNA, 119 bp, Bos taurus
  misc_feature (60)..(60): G OR A
ccaccattctcagtatgggtttcatcattaattattaaggtcatctcacagtccaagagnactgcctgaattccagtcagaaaatggattttctctctttctaggcagaaggaaaaaaa

SEQ ID NO: 545 - DNA, 119 bp, Bos taurus
  misc_feature (60)..(60): C OR T
actagtgatttgtatggtttccttattcttttaagttttctatccactttatcttacctngttagcttttgatggagttttgtttttgttttgaaatgtttttaagcttttatttagaa

SEQ ID NO: 546 - DNA, 119 bp, Bos taurus
  misc_feature (60)..(60): T OR G
ccaggttttccacatcagtgccaaccgttgttactctgtgtgtatgtgtggtggtggtgntcagttgctaagtcgtgtccaactctgcgaccccatagactgcagcatgacaggcttcc

SEQ ID NO: 547 - DNA, 119 bp, Bos taurus
  misc_feature (60)..(60): A OR G
ttgctattgaagaaacagaaggctttggacaagagtgaattctgagagcctgcaccaccnaaggacaagaatttatttgcaacgtttgtccttttccttctttgcctattgcacacttg

SEQ ID NO: 548 - DNA, 119 bp, Bos taurus
  misc_feature (60)..(60): G OR T
aaggtgataactgctctggggaaaattaagcaagtactaggagggaggatagtcactggngtgggaactgcaatattcagtaagatcattcaggaaggcttcccttgaggagaccttgg

SEQ ID NO: 549 - DNA, 119 bp, Bos taurus
  misc_feature (60)..(60): C OR T
tcctgggggattcccagaattgtgatagaaggccgccagtgcagttgaggccaagagggnaagagtgaggtagataaacctagagaagcaacagggaggaagatcctacaggcctcacg

SEQ ID NO: 550 - DNA, 119 bp, Bos taurus
  misc_feature (60)..(60): G OR T
agagtctattttggggaagatgatgggttcatcttgagtaggtcaagtctgagatgtctnctagacttccatctagagacataacacatgccaggcctttggattcagggaagatgcag

SEQ ID NO: 551 - DNA, 119 bp, Bos taurus
  misc_feature (60)..(60): C OR T
gggagacggcgcccctccgcgcccgcccctgcgcaggctgtgacgtcacaggactgtgangcgccctgggagcccccgccccttcgcggcgcggggccgggaacacaggctctctgcgc

SEQ ID NO: 552 - DNA, 119 bp, Bos taurus
  misc_feature (60)..(60): G OR T
aggtggggtatgggaccgggagctgggtccctgttctggggaggttgggaaaggtcaagntcaaggctggggtcagtgtccagtcagaggtaactgcctctagaagggcctgggatggg

SEQ ID NO: 553 - DNA, 119 bp, Bos taurus
  misc_feature (60)..(60): G OR C
ccattctgtcaccgcaggaactcatctagaaggctgatggaacccatcttacagacgagnaaactgaggcaccaagagttagttggcctaaggctgtactgggagagctggactggtgg

SEQ ID NO: 554 - DNA, 119 bp, Bos taurus
  misc_feature (60)..(60): C OR T
catggccaacagccctgggctgcagtgtcctggagaggcagggaaccccaggcatgtccnggttccccaggtctcattgtgttggggatgctgacacctgacagccacgactgggagaa

SEQ ID NO: 555 - DNA, 119 bp, Bos taurus
  misc_feature (60)..(60): CAGA OR C
gtgctatgaagggaacacacagaatgccaagacagagaccgtggggcagggaaacctcanagatctctctggggaggtgaccattaactgctatgaacttctaaatgtccatttgcact

SEQ ID NO: 556 - DNA, 119 bp, Bos taurus
  misc_feature (60)..(60): G OR A
ctgagtctgctgcaccagccagaagagaataaatgtgtctgcagttcctatggctcctcncgtcttcttccaccctcttagcttgcgccttgcctatcctgggttcaactaatagttcg

SEQ ID NO: 557 - DNA, 119 bp, Bos taurus
  misc_feature (60)..(60): TTA OR T
taatatgcaatttatatattctatatataatacagaatacatatcttatatattctatantatatatatattctatattatattatataacctaaaccatccctccccactccatctat

SEQ ID NO: 558 - DNA, 119 bp, Bos taurus
  misc_feature (60)..(60): A OR AC
ctgccagagcaactccgtcttaggggaaagaggaccaacgtccccagactcatcccatcncaaactagggtatgttcacacgttctgtgcctttgggacttgccaaggggccccaggct

SEQ ID NO: 559 - DNA, 119 bp, Bos taurus
  misc_feature (60)..(60): C OR G
tccgcagcaagagaccaccgcaatgacaagctagcgcatcctaactggagtgtagccccnactcgccacaaataaagaaaagcctgtatagcaacagagacccagcagaggcaaaagta

SEQ ID NO: 560 - DNA, 119 bp, Bos taurus
  misc_feature (60)..(60): G OR GA
ggaggtcaggtatcttcacaccttctgttcccacagctctgtggcccttggtgggggttngggggggggtgtcacaagatggcggtggcccccaggagctatctgtggcctttcacacg

SEQ ID NO: 561 - DNA, 119 bp, Bos taurus
  misc_feature (60)..(60): G OR A
gaggacaggtgtgctgggttccctgcttcttttagcccagacccaggcctgaccagaacncgggtaggacatatggaccacagacagctggagagcctggctgaggaaggctgccagaa

SEQ ID NO: 562 - DNA, 119 bp, Bos taurus
  misc_feature (60)..(60): G OR A
acattaaaaacatacatatggggttgaaacggatgtcaattcagttcagtcgctcagtcntatccaactcttcgcgaccccatgaatcgcagcacgccaggcctccctccctgtccatc

SEQ ID NO: 563 - DNA, 119 bp, Bos taurus
  misc_feature (60)..(60): C OR G
acgtactccttttcctatttggaaccagtctgttgttccatgtccagttctaactgttgnttcctgacctgcatacagatttctcaagaggcagctcaggtggagtggtattcccatct

SEQ ID NO: 564 - DNA, 119 bp, Bos taurus
  misc_feature (60)..(60): C OR T
gaagccatagactgcaggaatgaaatcccaggaactaagaaggaagaaggagaggcaaangactaccactagacttcccatatacatcatggcgtactcccaaaagaagatccaaaagg

SEQ ID NO: 565 - DNA, 119 bp, Bos taurus
  misc_feature (60)..(60): C OR T
aatatatagaacaaacatggacagagttacagaggaaaaatacacaaatccaccatcagnggataactggtagatctaatagacaaaaatcataatgaaatagaaatatgaatagcata

SEQ ID NO: 566 - DNA, 119 bp, Bos taurus
  misc_feature (60)..(60): T OR G
tccagatttctggtgagcagtgtggtcttgcataaaaaaactataaaatttctcacactnttggtgctaaaaatttagctggaagaacacaccttaaggaaattaaatgtctatactac

SEQ ID NO: 567 - DNA, 119 bp, Bos taurus
  misc_feature (60)..(60): T OR C
catgccccaactagaagttcccatgctgcaagtaaagatcccacatgtagctatgaaganagaagatcctgtgcaccacaactaagtcctggctcagccaaataattaattaattaatt

SEQ ID NO: 568 - DNA, 119 bp, Bos taurus
  misc_feature (60)..(60): G OR C
agctacaattagccaccaggtctgtctcactaaaaaaaatgcacaacctaaaaagttganatttatattttatttggtggacaagactgaggacttaagcttgagacaaaccatctcag

SEQ ID NO: 569 - DNA, 119 bp, Bos taurus
  misc_feature (60)..(60): C OR T
tggaggctggaacccagatcagcatcgagtgaaaaagcatctgtgaagggttgattgtgngtgtgcctgtgtgaggctttgtcccaaaagagacagaatggctctgtctcttttcctgg

SEQ ID NO: 570 - DNA, 119 bp, Bos taurus
  misc_feature (60)..(60): C OR T
tggtccagtggctaagattctgagctcccaatgcagagggcccaagttcgatccctggtnagggaactagatcccacatactgcaacgaagacctggtgcaggcaattaagtaaataaa

SEQ ID NO: 571 - DNA, 119 bp, Bos taurus
  misc_feature (60)..(60): T OR A
ctttgggagcttctcctttgttatgatatgaatgatcaagtggtgtgtctaagcaacacntgtggctgggaagcatggttagcagtgggggaataatcaaccttaggctccaccctcca

SEQ ID NO: 572 - DNA, 119 bp, Bos taurus
  misc_feature (60)..(60): G OR A
actagaagaactaaaatatacaccataaatactgctgaattgtaggaatactggctaacngataacattcttcttcataattctctgatctgaatttcaatttatctttagtaaactct

SEQ ID NO: 573 - DNA, 119 bp, Bos taurus
  misc_feature (60)..(60): G OR A
caaaaaaccagtcaaaaggatgcctcttctgagaagtcttccatgacctcagtccccaanaggagttgccccttctgccttttgggttcagagtctgtgtctggcattctagagcaccc

SEQ ID NO: 574 - DNA, 119 bp, Bos taurus
  misc_feature (60)..(60): T OR C
taatggaggtgcctctttctcctgctaaaacaaaagcctcagcaatactgtcctggtgtntatagggtcaatggtcaggcgactcagggaacaaacacctataaggtatctttggtctg

SEQ ID NO: 575 - DNA, 119 bp, Bos taurus
  misc_feature (60)..(60): G OR C
ctacagttgtatttcatcttgttaggttatgtgggacagcacagtataaaagacaaagcntgagttttgtggtcagagcccatctgcctttctctcacttgaggatcaaacagctacac

SEQ ID NO: 576 - DNA, 119 bp, Bos taurus
  misc_feature (60)..(60): C OR T
ttaaaataatcacaacttctaccttacattttaaaaagttttttattcatttatttattngactgctctgttctggtgtggcttgtggcattttagttccctgaccggggaatgaactg

SEQ ID NO: 577 - DNA, 119 bp, Bos taurus
  misc_feature (60)..(60): TATAC OR T
attttatgaggacctaatataacaaagactaatttgagcaaaatacacacacaaatacanatacacgtgcacatacacacactttcagaggttatatttgaaactttttacttactaac

SEQ ID NO: 578 - DNA, 119 bp, Bos taurus
  misc_feature (60)..(60): C OR T
tatctttagggtagctatcaaattcaaccagggaatccagacttctttttttgtgaatantgcaacggtggctccaacaatccataacaccttgcaagaaattgtagactggctaatga

SEQ ID NO: 579 - DNA, 119 bp, Bos taurus
  misc_feature (60)..(60): A OR G
cccaccagccatcagttttcaatttctaattaagtctagtcagtttctaattaagtctantaagtctaagtctaacctggattgagttaaatccaagtataacttacaaaactggtaga

SEQ ID NO: 580 - DNA, 119 bp, Bos taurus
  misc_feature (60)..(60): C OR T
ctgcctgcaatgcaggagacccaggttcaatccctgggtggggaagatcccttggcgaanggaacggctacccgctccagtattcttgcctggagaatcccacgaacagaggagcctgg

SEQ ID NO: 581 - DNA, 119 bp, Bos taurus
  misc_feature (60)..(60): C OR T
aatcggaagagaagcacttcctggcttcggacccaaatcacagaggtttcctctcccctntccatcaacctcatgcctttccctccacccccaggccccactgacctagatcatgggtc

SEQ ID NO: 582 - DNA, 119 bp, Bos taurus
  misc_feature (60)..(60): G OR A
agagttaggattccaagcctagactgtctccaaagcatatactttgttctacttcttggnaatggcattcaggctatttctcctaaggtttggcaaagacgtaactgtcttcctgtgag

SEQ ID NO: 583 - DNA, 119 bp, Bos taurus
  misc_feature (60)..(60): C OR T
tagctggcatctattgatctctcagtgccagccagacactggggaagggctcagtaatcnctcaaggaggaagttacaaccatccctgaagtatgaagaaattgaggctcgggatgaac

SEQ ID NO: 584 - DNA, 119 bp, Bos taurus
  misc_feature (60)..(60): C OR T
tgggtttcctgcattgcaagtggatgcttttaccaactgagccaccagggaagtgactcnattcagtaggttgtcactttgttttatcaatggtttcctttgcaatgcgaaagcttgta

SEQ ID NO: 585 - DNA, 119 bp, Bos taurus
  misc_feature (60)..(60): C OR G
ctcgtccctgtacctgttgaagaaactgggtcatttgtcctgagtataggcattagactnaactagattcgggttcacttttttttatatataattgtttcttttttattggagtatag

SEQ ID NO: 586 - DNA, 119 bp, Bos taurus
  misc_feature (60)..(60): A OR G
gcatctctggctcccagcctagaggcaaatagacaagtaaggtggcaacaaaggcagtangggaaggcacttgggagagagcatctgagcagagggccctgtgagagggggttgtcagg

SEQ ID NO: 587 - DNA, 119 bp, Bos taurus
  misc_feature (60)..(60): G OR A
ctaggtctcagcttccatatttatattataggaaagatgatacctacggtcctagaattntatacctaccagaatctatcctgtagggtattgtgagtgaggatgaatgatgagaagta

SEQ ID NO: 588 - DNA, 119 bp, Bos taurus
  misc_feature (60)..(60): G OR A
tttagggctggcatgctgaggactaaatcctcacaaggcctgagtgccacacagagtgtnttctatccagtcacatccttctgtctgcttccctttattgctcctgagtaccttccgtg

SEQ ID NO: 589 - DNA, 119 bp, Bos taurus
  misc_feature (60)..(60): C OR T
gagatggctgtctgtggaagttagtcatttactctcttgacaaggcattcaaacacatcngcaagcccgtaatcaccctgaaaagaagctgatggggtacagtaaggtcctgggaagat

SEQ ID NO: 590 - DNA, 119 bp, Bos taurus
  misc_feature (60)..(60): A OR G
tgtatactgtcaccctgcttatttaacttctatgctgggctggatgaatcaccagctggnattaagattaagatatcaacaacctcatatatgcagatgataccaccgtaatggtagaa

SEQ ID NO: 591 - DNA, 119 bp, Bos taurus
  misc_feature (60)..(60): A OR T
atgaggaaactaagacccagggagaggctgcaacttaaggtagttttaggacaaaaaaantttttttaaagtttccatttacaagatgaggagaaaatttacccaaggagcatgaacat

SEQ ID NO: 592 - DNA, 119 bp, Bos taurus
  misc_feature (60)..(60): G OR T
ggcagaatattgaaagcatgctccaaaatgcacacagagcccctcaacaaatactgaganacttactggttccacatttaaggaaatctccatccaatcattacttgaccactaagcta

SEQ ID NO: 593 - DNA, 119 bp, Bos taurus
  misc_feature (60)..(60): T OR G
ttgaaacagagtgcagaggtactgataggagatgaggtcagatcatgccagcaccacaanaagtatggcctcacgagccagagaaaagagggtggattttatttttagtgcagtgggga

SEQ ID NO: 594 - DNA, 119 bp, Bos taurus
  misc_feature (60)..(60): C OR G
cacccactgccatccatgggctcaggccgctgagctggggttcccagttcctcctcctcntccactggcccgactcccatgtctgactccagcaggagctgtttctccccttctgtgtc

SEQ ID NO: 595 - DNA, 119 bp, Bos taurus
  misc_feature (60)..(60): C OR T
gtagggataggttgctgggtggagggaagacttttccagaaatcagcttgccaattttcnccttccctcctctcttctgtcactcgtgggagacgaccaggggcacagaacacggcagc

SEQ ID NO: 596 - DNA, 119 bp, Bos taurus
  misc_feature (60)..(60): T OR C
gacacagtcagtgtgaagtgggttccaagcagtgggccataggagagggctgaggggtcncaggccttacctgagccctgggagggaggctggcaggccggcagaccggtcatatgggg

SEQ ID NO: 597 - DNA, 119 bp, Bos taurus
  misc_feature (60)..(60): G OR A
gtggctgagggacatgctagccagctcggctcaaaagtcctgcagctgagggatgtcccngtacaaatccagggtctcgggattcgagaaggcccctcggtgctccaccgtcttcccag

SEQ ID NO: 598 - DNA, 119 bp, Bos taurus
  misc_feature (60)..(60): T OR C
acaggtgtgatgtggggctgggggcagaactgaactaacagttctcacccccaaaggcangcctcctagtggggtccaccctacacccagttctgagatgcagcggtgcgtgagaaaca

SEQ ID NO: 599 - DNA, 119 bp, Bos taurus
  misc_feature (60)..(60): A OR G
gaggaggcagggatcagagctttaaataaagagacttcccagctcaggggcagggtgccnctccaaagatgagcgaagagggcagcctgagtgagtgacgggcctgaaattaattatcc

SEQ ID NO: 600 - DNA, 119 bp, Bos taurus
  misc_feature (60)..(60): T OR C
ggttaacactccacagcttccagtacagggggcatgggttcgatccctggtaggggaacnaagatcctacatgccatgtggtgtggccaaaaaaataaaacctccaggtctcacctctc

SEQ ID NO: 601 - DNA, 119 bp, Bos taurus
  misc_feature (60)..(60): T OR C
catgttgccatgaagaatgtgttccttcaggtgctggatgagcgtcccctggggagaagngagtgagccaagcagcttcctccggggcacaaaggcacgcgtggaatagacactgggcc

SEQ ID NO: 602 - DNA, 119 bp, Bos taurus
  misc_feature (60)..(60): T OR C
atccaaccatctcatccccttctcctgccttcaatctttcccagcatgagggtgttttcnaatgagtcagctccttgccttaggtgaccaaagtatgaatcttaaggagtcaaatgaat

SEQ ID NO: 603 - DNA, 119 bp, Bos taurus
  misc_feature (60)..(60): A OR G
tttagagatctcttctgactcgtgaaggtcacagcaagaaagtgggatcaaggtcccacnagagtttctatgacagaaatcctgccagacaatgatcctgttctcactgattgcctata

SEQ ID NO: 604 - DNA, 119 bp, Bos taurus
  misc_feature (60)..(60): T OR A
gaagaaagtactattattcccattttacagatggggaaacccaggcacagcaaggttgancgctagcccagggccgcacagctgacatacgacccagagcagtctggctggaagccaca

SEQ ID NO: 605 - DNA, 119 bp, Bos taurus
  misc_feature (60)..(60): A OR C
ctcctgctaatgcaggggtcacgggttcaatccctggtccaggaagatccatcatgctgnggagcaacttagctcatgcacctcagctatcgagcccacacaccctagagcccttactc

SEQ ID NO: 606 - DNA, 119 bp, Bos taurus
  misc_feature (60)..(60): C OR T
ctaggaccagggaggaggccaagagcccaggccatggggcatctatgacagcacagatcngggcaaagtaggaagagtcagcacagctgtgatgaggggaaggggaggccttagggctc

SEQ ID NO: 607 - DNA, 119 bp, Bos taurus
  misc_feature (60)..(60): C OR A
gaagccaatgaccacattgggaggaactcaagcagtcctaggtggcaagtagcggaggcnccctgcccacaaccagcaatgactcactgccatgcgagccagcccctgaatagtgtggc

SEQ ID NO: 608 - DNA, 119 bp, Bos taurus
  misc_feature (60)..(60): G OR A
gcagagcgctcagcctgatggccttcacgcccagccaatgatgtcagtcctctcattatntccattttacagatgagggaaaccaaggctcgggagatccagtgacttccctctgacca

SEQ ID NO: 609 - DNA, 119 bp, Bos taurus
  misc_feature (60)..(60): T OR C
aggaccgggagcaggccggggaccgagccgggggcggcagcagaggccgggagcgggacnccgggcgagcggcgaggcagacgtggagcaactgcacgcggagcagccgccccgcccga

SEQ ID NO: 610 - DNA, 119 bp, Bos taurus
  misc_feature (60)..(60): A OR G
gtgtaggctttgcagatacatcaccgagcaaagcgttaattcctgccctcagtcaacagnagataaggggtaggggacaggtgtggggaggagcggggtcaatcaacaaatacattata

SEQ ID NO: 611 - DNA, 119 bp, Bos taurus
  misc_feature (60)..(60): G OR A
ttatgggtggagaaggcaatggcaacccactccagtattcttgcctggagaatcccaggnatggaggagcctggtgggctgccgtctatggggtcacacagagttggacacaactgacg

SEQ ID NO: 612 - DNA, 119 bp, Bos taurus
  misc_feature (60)..(60): A OR C
tacacctgacagctacttggttcattcttatttctttcagctcagagccccccacccccncccccatagaggcacaggacctgtaggggaagccaagggagaagtaagctacagtcaaa

SEQ ID NO: 613 - DNA, 119 bp, Bos taurus
  misc_feature (60)..(60): T OR C
accaagaatcttaaactttctgtgggtaacccctcttaggtctctgaaattcccatcttncccatcttaccagttgctttcttttttcttttttggctgcactggctcatggttgctaa

SEQ ID NO: 614 - DNA, 119 bp, Bos taurus
  misc_feature (60)..(60): G OR A
ggcctgaggcatgcttggcacagtcaaaaggtagtgaccatacgtatagggccttgggangctctagggtaaggacttcagatttcattcaaagtgcaatcagaagccacttggaaagt

SEQ ID NO: 615 - DNA, 119 bp, Bos taurus
  misc_feature (60)..(60): C OR T
caaaagcttttcagacaagcaaaagctgagagaattctgcaccacctacatgtctatatncttaaagagatgtgaataccagaccacctgacctgccagttgagaaatctgtatcctgg

SEQ ID NO: 616 - DNA, 119 bp, Bos taurus
  misc_feature (60)..(60): C OR A
ccatgtgccacaactaaagatcctgcatgctgcaactaaggcccagtgcagccaaataantaagtattttttaggaaaggaagatgaagccataaaatctgtttgtttgattttttaag

SEQ ID NO: 617 - DNA, 119 bp, Bos taurus
  misc_feature (60)..(60): A OR G
gcaagctccgggagttggtgaaggacagggaggcctggcatgcagcagtccatggggtcncaaagagtcggacacgactgagcaactaaactgagctgactcgaagaaaggcaaataca

SEQ ID NO: 618 - DNA, 119 bp, Bos taurus
  misc_feature (60)..(60): G OR A
cactcatctcacatgctagtaaagtaatgcttaaaattctccaagccaggcaaaacatgnaccatgaacttccagatgtttaagctggttttagaaaaggcagaggaaccagagatcaa

SEQ ID NO: 619 - DNA, 119 bp, Bos taurus
  misc_feature (60)..(60): G OR A
gttagaggaaacttttgggattaaagattttgattcgatatgtacttataagagagtatnttaagttgcatgataaaaatgcaactttaaacagaaaaccttgtgaatctgtgactgta

SEQ ID NO: 620 - DNA, 119 bp, Bos taurus
  misc_feature (60)..(60): A OR G
acaatgggcatgctgctgctactgctgctaagtcgcttcagtcgtgtccgactctgtgcnaccccatagacggcaacccaccaggctcccccgtccctgggattctccaggcaagaaca

SEQ ID NO: 621 - DNA, 119 bp, Bos taurus
  misc_feature (60)..(60): A OR C
gtcgcttcagtcgtgtccgactctgtgcgaccccatagacggcaacccaccaggctcccncgtccctgggattctccaggcaagaacactggagtgggttgccatttccttctccagtg

SEQ ID NO: 622 - DNA, 119 bp, Bos taurus
  misc_feature (60)..(60): T OR A
caaccacttgcataaaagtaatagaaacacaatggggacttttttttttctttttttttnaattttatttaatttttaaactttacataattgtattagttttgccaaatatcaaaatg

SEQ ID NO: 623 - DNA, 119 bp, Bos taurus
  misc_feature (60)..(60): G OR A
acaaataataaatgctggagagggtgtggagaaaagggaaccctcttacactgttggtgngaatgcaaactagtacagccactatggagaacagtgtggagattccttaaaaaactgga

SEQ ID NO: 624 - DNA, 119 bp, Bos taurus
  misc_feature (60)..(60): G OR A
caaataataaatgctggagagggtgtggagaaaagggaaccctcttacactgttggtggnaatgcaaactagtacagccactatggagaacagtgtggagattccttaaaaaactggaa

SEQ ID NO: 625 - DNA, 119 bp, Bos taurus
  misc_feature (60)..(60): C OR T
agactcttgagagtcccttggactgcaaggagatccaaccagtccatcctaaaggagatnagtcctgggtgttctttggaaggaatgatgctacagctgaaactccagtactttggcca

SEQ ID NO: 626 - DNA, 119 bp, Bos taurus
  misc_feature (60)..(60): T OR C
tctataccctgtgtgaaaacagatctgtctctaagtacctgctgctgctgctgctaagtngcttcagtcgtgtccaactctgtgcgaccccatagatggcagcccaccaggctcctgcg

SEQ ID NO: 627 - DNA, 119 bp, Bos taurus
  misc_feature (60)..(60): T OR C
atccccccacccttttcttttcctggctgcaccatgtagtacgtgagatctccgttcccngataaaggattgaacccgcaccctcagcagtgaaagctcagagtcctaaccgctagacc

SEQ ID NO: 628 - DNA, 119 bp, Bos taurus
  misc_feature (60)..(60): TACACACACACACACACACACACACACACAC OR T
gagttgaattctctgtcaccgcatggtatctgcacattgctagctggtggggggaagccnacacacacacacacacactttggaatttggtccaggaacctttttcagtcgcacgccta

SEQ ID NO: 629 - DNA, 119 bp, Bos taurus
  misc_feature (60)..(60): C OR T
tttttaacatggcaattagaacgtttaacgtcacatatgtggctggcattctatttctanggaaccactccaggctaaggactctccattttaaccagtaccttgaaacattctgaagc

SEQ ID NO: 630 - DNA, 119 bp, Bos taurus
  misc_feature (60)..(60): T OR C
gaatacggtggttcaaagccagagggtgggttcccagggatggaggggatgacagtgggngcgtaccagggtatgggtttgggccagcagccagctcaactccaaggactcggtctctg

SEQ ID NO: 631 - DNA, 119 bp, Bos taurus
  misc_feature (60)..(60): T OR C
ggctcaagtcgctccagctggtccctggctcatggactgcacccctccttctctgtctcngttttcacattcacatggccatcatctctgtgtgtctgtgtctctgtccaaatttccct

SEQ ID NO: 632 - DNA, 119 bp, Bos taurus
  misc_feature (60)..(60): C OR T
ccttgctcacggatctcagaggggagatgagtaacaaacacacacacgtgggagaagganaggaggtggaagaatgtgagtgtgagaactgagcatgaggttcctcacacaaggtggtt

SEQ ID NO: 633 - DNA, 119 bp, Bos taurus
  misc_feature (60)..(60): AAC OR A
aaccaagcatgggaaagacaggagcaggcaaagggctgggaagatcgtccccagcagggnacagagactggaaggcgtcaagctgggtatgtccaggggctaggcagcatccttggtgt

SEQ ID NO: 634 - DNA, 119 bp, Bos taurus
  misc_feature (60)..(60): A OR G
ggctgggaagatcgtccccagcagggaacacagagactggaaggcgtcaagctgggtatntccaggggctaggcagcatccttggtgtgaactttgtcaattgaaagtgcatcaggaaa

SEQ ID NO: 635 - DNA, 119 bp, Bos taurus
  misc_feature (60)..(60): A OR G
gctaggcagcatccttggtgtgaactttgtcaattgaaagtgcatcaggaaatcactggncagttttaacgagagtgacttaatctgacttttacattttgtttttttaaaatcactaa

SEQ ID NO: 636 - DNA, 119 bp, Bos taurus
  misc_feature (60)..(60): A OR G
tgtgtttgttaatcaggtggttctgaaaagcctctaaaaatgtaggctggttgccaaggnaaccaacttggttattagagggttgagcctttcgattgaccctcctgacctcttgggag

SEQ ID NO: 637 - DNA, 119 bp, Bos taurus
  misc_feature (60)..(60): A OR C
tgggactaagcctttgacccgtaggatcagatgctacctccagggaggcagggtcagaantgagttcaatctgggcctggaggttttgcaaacaacgtggttcacactgatatatgctt

SEQ ID NO: 638 - DNA, 119 bp, Bos taurus
  misc_feature (60)..(60): T OR C
cccttctccaggggatcttcctgacccagggattgaagccaggtctcctgcattgtaggnggattctttaccatctgagccaacaggcaagtctagggacagacaagttggcaataccc

SEQ ID NO: 639 - DNA, 119 bp, Bos taurus
  misc_feature (60)..(60): T OR C
aaccccagggcttggccaaatcgccctgtggattaatggagcataggactagcttggggngccaagaaccaagtgaatatggtcaaatctccctcctggacttccctggtggtccagtg

SEQ ID NO: 640 - DNA, 119 bp, Bos taurus
  misc_feature (60)..(60): G OR C
gaagcccatgagccctagagcccgtactctgcaaaaagagaagccactgcaatgagaagnctgcacactgcagctagagtagccccagctctcgaaactacagaagagctgcctgcagc

SEQ ID NO: 641 - DNA, 119 bp, Bos taurus
  misc_feature (60)..(60): T OR G
ccagaagccctctgcaaaagtgctcactacacttctgattactcccagagccctctgctnactcttgtcaaagtccctgcctgaattctcatttctaaatggtgaaggtgttgcaacca

SEQ ID NO: 642 - DNA, 119 bp, Bos taurus
  misc_feature (60)..(60): T OR C
cctgaattctcatttctaaatggtgaaggtgttgcaaccaacctatttcacccagaggcngggtgctgctgcatgggagcaactctaaagggcgagaacggcggttctcacagttgagt

SEQ ID NO: 643 - DNA, 119 bp, Bos taurus
  misc_feature (60)..(60): A OR G
caaccaacctatttcacccagaggccgggtgctgctgcatgggagcaactctaaagggcnagaacggcggttctcacagttgagtcccatcgtcacatctcgggcacgtgcaaacgaca

SEQ ID NO: 644 - DNA, 119 bp, Bos taurus
  misc_feature (60)..(60): T OR C
aacctatttcacccagaggccgggtgctgctgcatgggagcaactctaaagggcgagaanggcggttctcacagttgagtcccatcgtcacatctcgggcacgtgcaaacgacagagac

SEQ ID NO: 645 - DNA, 119 bp, Bos taurus
  misc_feature (60)..(60): A OR C
acagggacttcctggtggtccagcagataagacgactctgagctgccaatgcaggggggncagggtttgattcctggtcagggaactaggatcctgcctgccctgcagtgtgaccaaaa

SEQ ID NO: 646 - DNA, 119 bp, Bos taurus
  misc_feature (60)..(60): T OR C
tagcactcaagcttagttgccctgtggcatgtggaatcttattggaccaggggtggaacncatgtcctctgcattggcaggtggattcttaaccactggagcaccagggaagtcctcaa

SEQ ID NO: 647 - DNA, 119 bp, Bos taurus
  misc_feature (60)..(60): G OR GT
gatgtatcatttacatactataaggttctcgtctttaaaatgcatgattcagtgttgttntttttttttaaaaaatcccccataattgaattttagaacatcttcatctccccaaaagg

SEQ ID NO: 648 - DNA, 119 bp, Bos taurus
  misc_feature (60)..(60): G OR GAAAGTGA
ccctgggattctccaggcaagaacactggagtgggttgccatttccttccccaatgcatnaaagtgaaaagtgaaaagtgaaagtgaaatcgctcagtcatgtccgactcttagcgacc

SEQ ID NO: 649 - DNA, 119 bp, Bos taurus
  misc_feature (60)..(60): C OR T
tgtccgactcttagcgaccccatggactgcagcctaccaggctcctctgtccatgggagnttccaggcaagagtactggagtgggttgccagtgccttctccctctcagcctaggcaac

SEQ ID NO: 650 - DNA, 119 bp, Bos taurus
  misc_feature (60)..(60): T OR C
gagtgggttgccagtgccttctccctctcagcctaggcaaccactaatcttcttcccgtntccacggttttgcctttcctggactttctgtacaaatgaaatcaggcaatccgtgacct

SEQ ID NO: 651 - DNA, 119 bp, Bos taurus
  misc_feature (60)..(60): A OR C
gttttgcctttcctggactttctgtacaaatgaaatcaggcaatccgtgaccttttgtgnctggcttcttgcactcaaaataataatgtctggcttcacccatgtggctgcgtgaatca

SEQ ID NO: 652 - DNA, 119 bp, Bos taurus
  misc_feature (60)..(60): C OR T
tcttgccaaatagcactcccttccagggttctccacgctttgttcctcctctcgttattntttaaaagcatcacagtttgtgcccatttgtccataggtagcaatttttacattttgtt

SEQ ID NO: 653 - DNA, 119 bp, Bos taurus
  misc_feature (60)..(60): GT OR G
ctattttgggaagaagggacattacagcttctgctgccgctgaccttctctgtgccttanttctcagctgtaaaatgggactgaggaagaatggacattagggaacttccctgtccacc

SEQ ID NO: 654 - DNA, 119 bp, Bos taurus
  misc_feature (60)..(60): C OR T
gctcagaggaaaggtgaggcccaccttggggaccagccagggcaggaaagggtgatacanggaaaggctcatcgggggccttttggaccgggtgggtgggaacactctgccccctgggg

SEQ ID NO: 655 - DNA, 119 bp, Bos taurus
  misc_feature (60)..(60): A OR G
tgaggcccaccttggggaccagccagggcaggaaagggtgatacatggaaaggctcatcnggggccttttggaccgggtgggtgggaacactctgccccctggggctggcaccgctcac

SEQ ID NO: 656 - DNA, 119 bp, Bos taurus
  misc_feature (60)..(60): C OR G
cctggggctggcaccgctcactgtggcttctgctccagcaagttagtccttggctccctntctgtgtgaagacacaggagccctgaggggatgctgtgggcagcactgtgttcctccac

SEQ ID NO: 657 - DNA, 119 bp, Bos taurus
  misc_feature (60)..(60): A OR C
gtctgtgtgaagacacaggagccctgaggggatgctgtgggcagcactgtgttcctccanatctcagtgaaggtgaactgaggccaatgtggaggacctggatccaacaggactggggg

SEQ ID NO: 658 - DNA, 119 bp, Bos taurus
  misc_feature (60)..(60): C OR T
gtgtgaagacacaggagccctgaggggatgctgtgggcagcactgtgttcctccacatcncagtgaaggtgaactgaggccaatgtggaggacctggatccaacaggactgggggccct

SEQ ID NO: 659 - DNA, 119 bp, Bos taurus
  misc_feature (60)..(60): C OR T
gcactgtgttcctccacatctcagtgaaggtgaactgaggccaatgtggaggacctgganccaacaggactgggggccctacaagaagagatggggggcttccccacaggtcccccagt

SEQ ID NO: 660 - DNA, 119 bp, Bos taurus
  misc_feature (60)..(60): G OR GT
ggacctggatccaacaggactgggggccctacaagaagagatggggggcttccccacagntcccccagtggttagaactcggtgtggtcagtgctacaggcgcaggttcagtcctggtc

SEQ ID NO: 661 - DNA, 119 bp, Bos taurus
  misc_feature (60)..(60): C OR G
gggcttccccacaggtcccccagtggttagaactcggtgtggtcagtgctacaggcgcangttcagtcctggtcagagaactaggatcccgcatgctgagcagcgtggcaaaaaaaagg

SEQ ID NO: 662 - DNA, 119 bp, Bos taurus
  misc_feature (60)..(60): TC OR T
cccacaggtcccccagtggttagaactcggtgtggtcagtgctacaggcgcaggttcagnctggtcagagaactaggatcccgcatgctgagcagcgtggcaaaaaaaaggttaacact

SEQ ID NO: 663 - DNA, 119 bp, Bos taurus
  misc_feature (60)..(60): T OR A
tggtcagtgctacaggcgcaggttcagtcctggtcagagaactaggatcccgcatgctgngcagcgtggcaaaaaaaaggttaacactgcatttgatgggaaaaaaaattttttttaaa

SEQ ID NO: 664 - DNA, 119 bp, Bos taurus
  misc_feature (60)..(60): A OR G
aggttcagtcctggtcagagaactaggatcccgcatgctgagcagcgtggcaaaaaaaangttaacactgcatttgatgggaaaaaaaattttttttaaaagaaagaaaatgaacccac

SEQ ID NO: 665 - DNA, 119 bp, Bos taurus
  misc_feature (60)..(60): A OR T
tgagcagcgtggcaaaaaaaaggttaacactgcatttgatgggaaaaaaaattttttttnaaagaaagaaaatgaacccactccagtcttcttgcccaggaaatcccatggacgaagga

SEQ ID NO: 666 - DNA, 119 bp, Bos taurus
  misc_feature (60)..(60): A OR G
tttttaaaagaaagaaaatgaacccactccagtcttcttgcccaggaaatcccatggacnaaggagcctggtgagctacagtccaaggggctgcaaagagtcaggcaccaccatgccca

SEQ ID NO: 667 - DNA, 119 bp, Bos taurus
  misc_feature (60)..(60): G OR A
cttgcccaggaaatcccatggacgaaggagcctggtgagctacagtccaaggggctgcanagagtcaggcaccaccatgcccatgtttaactgaatctctgctgtacacctgaaactaa

SEQ ID NO: 668 - DNA, 119 bp, Bos taurus
  misc_feature (60)..(60): A OR G
gttcagtgaagcccagagcctgaagcagttcacagaaataggtgaaaagccaaaccaacngcacaggttgtatgttttttctacaacctataaaatatagccaaaattttatagtaact

SEQ ID NO: 669 - DNA, 119 bp, Bos taurus
  misc_feature (60)..(60): A OR G
attttatagtaactataaatgaagtataacctataaaaagtgtgaatgttgtataccgcnaacagcactgtataatcatatactactgtacatcaactctacctcaattcaaaaatgta

SEQ ID NO: 670 - DNA, 119 bp, Bos taurus
  misc_feature (60)..(60): A OR C
ggttcatcatacaattctttctccctttgtgtctcctgggcaatattcaggatgaacagntaaaatgaaaaagattcaagtgagccaactcaggaggcccgcggccatggccagtccaa

SEQ ID NO: 671 - DNA, 119 bp, Bos taurus
  misc_feature (60)..(60): G OR A
ggccatggccagtccaagggagaagacacaattactccagctcagcatttcatcaccccntcctaaatctggagggcatcctaaactctcctcacccccattacccccaagcatattta

SEQ ID NO: 672 - DNA, 119 bp, Bos taurus
  misc_feature (60)..(60): A OR G
gctgggtaactgaggtaccatgtgatatatgctgaggccaaaaagtaagataaaccacgnggagaaaccatgtcagtcctactagaatggttacactcaaaaagtgtaacctacgtcaa

SEQ ID NO: 673 - DNA, 119 bp, Bos taurus
  misc_feature (60)..(60): T OR C
gaggtaccatgtgatatatgctgaggccaaaaagtaagataaaccacggggagaaaccangtcagtcctactagaatggttacactcaaaaagtgtaacctacgtcaacaggtgaatgg

SEQ ID NO: 674 - DNA, 119 bp, Bos taurus
  misc_feature (60)..(60): C OR T
caactctgagcttccactgcacggggtgcaggtcctggttggggaactaggacctgacangctgtgaggcaaggtcaaagagaaagacaactactgtatgataacactttgtggggaat

SEQ ID NO: 675 - DNA, 119 bp, Bos taurus
  misc_feature (60)..(60): T OR A
agaggcagactcacagatacagagaacaaactagcggttaccagcagggaaaggaaagangggggatggacaagataggggtagaggctaagagatacaactactatgtataaagtaag

SEQ ID NO: 676 - DNA, 119 bp, Bos taurus
  misc_feature (60)..(60): A OR T
tgctgataggaacgtcaaatggtctgaacagaaactccttgagatgtcagagctgccatntgctgtgccgtgtgcttactcactcagctgcccaactctttgcgaccccatggactgtc

SEQ ID NO: 677 - DNA, 119 bp, Bos taurus
  misc_feature (60)..(60): C OR T
aaacattttggaccctaacttgatgtgacctccacatctaactcacagtttacaggaaanatgggggatggaagcagaaaaattagaaacagcagaagctaaagtcagaatgtggaacg

SEQ ID NO: 678 - DNA, 119 bp, Bos taurus
  misc_feature (60)..(60): T OR C
tacccaggtcaagaaaaggaacattatcagttttgccaaaaggctccctttgccctctcntgttcactacaccccctccaaaatttaggggattcattttgcctgtttttgaacttctg

SEQ ID NO: 679 - DNA, 119 bp, Bos taurus
  misc_feature (60)..(60): T OR G
tctcctcctgtcttcagtctttctcagcatcagggtctttttcaatgtgtcaattcttcncatcaggtggccaaagtattggagcttgagcttcagtatcagtccttccaatgaatatt

SEQ ID NO: 680 - DNA, 119 bp, Bos taurus
  misc_feature (60)..(60): A OR G
gaatatatagaatattttgcatacattcatactaaacagataaagtacaagaggtgtttngtgagttgcagagtttcgtgcgatacagagggaagaccccattgagaaagcattggatc

SEQ ID NO: 681 - DNA, 119 bp, Bos taurus
  misc_feature (60)..(60): C OR T
tgcatacattcatactaaacagataaagtacaagaggtgtttggtgagttgcagagtttngtgcgatacagagggaagaccccattgagaaagcattggatccttaagacctgagggcc

SEQ ID NO: 682 - DNA, 119 bp, Bos taurus
  misc_feature (60)..(60): G OR A
cgatagggaaagtatcttctgggataagagctcggtgaccagctgagaacaggggggccntcccatactgagaactgcgtgagcaaaggccctgaggcaagagggcgcatgggtctcag

SEQ ID NO: 683 - DNA, 119 bp, Bos taurus
  misc_feature (60)..(60): AGAGCCT OR A
taacagaccttgaagtgccactggcttcacgccacaaaggtttccttgtaggtgatgtcnacccactggcaagtggggggtatgggattctggtccacacactgattcagcgacccagg

SEQ ID NO: 684 - DNA, 119 bp, Bos taurus
  misc_feature (60)..(60): C OR T
ctctttcccattgctcctttggcactgcccacttcctcctggccaccctctggcttcacngatcctcacagtccacccaccgccaccctgcccccggcggcctcccgcttcaagccctg

SEQ ID NO: 685 - DNA, 119 bp, Bos taurus
  misc_feature (60)..(60): T OR C
gcactgcccacttcctcctggccaccctctggcttcactgatcctcacagtccacccacngccaccctgcccccggcggcctcccgcttcaagccctgacagtgtcccggccctccccc

SEQ ID NO: 686 - DNA, 119 bp, Bos taurus
  misc_feature (60)..(60): T OR G
aacactatcctcgcccacccgcccccctgggtgccagagacttgctgactcctgccaaantcggcatggtcaccccctgcctcagggccaaactacggctccagcagcactgactggtc

SEQ ID NO: 687 - DNA, 119 bp, Bos taurus
  misc_feature (60)..(60): A OR G
ctgggtgccagagacttgctgactcctgccaaagtcggcatggtcaccccctgcctcagngccaaactacggctccagcagcactgactggtccagccactgtcaccccttgggtcctc

SEQ ID NO: 688 - DNA, 119 bp, Bos taurus
  misc_feature (60)..(60): C OR G
acagcttcactgtagcagcagcagcagcaaagactgtcagcacccctcacgggccccagngcagcagcatccagaccacaggaagtccccaggaccactggctgacgatgggccttcag

SEQ ID NO: 689 - DNA, 119 bp, Bos taurus
  misc_feature (60)..(60): T OR C
ctcttaacaggaaactgaaagcagaccctgtgcctgtgggctcaaacgctggtgggagcncgcctaccccgggtcagccaggtttctgcccccaagtctgtcactgaatctttctccgc

SEQ ID NO: 690 - DNA, 119 bp, Bos taurus
  misc_feature (60)..(60): G OR A
gcagaccctgtgcctgtgggctcaaacgctggtgggagcccgcctaccccgggtcagccnggtttctgcccccaagtctgtcactgaatctttctccgcatttccagcctggatgggaa

SEQ ID NO: 691 - DNA, 119 bp, Bos taurus
  misc_feature (60)..(60): A OR G
tctgcccccaagtctgtcactgaatctttctccgcatttccagcctggatgggaagggcntttgggggagaacggatccatgtatgtagatggctgagtcccttccctgctcacctgaa

SEQ ID NO: 692 - DNA, 119 bp, Bos taurus
  misc_feature (60)..(60): GAAAAAA OR G
tgaaactatcacaacattgttaactggctacaccccaacacaaaacaaaaaattcgaaanaaaaacttcctccacttttcatactgttaacctttcctgttttcaactttgtttttcct

SEQ ID NO: 693 - DNA, 119 bp, Bos taurus
  misc_feature (60)..(60): A OR C
ctctttttttcaaaatgcttcagttatatcaggcacataaaaatgtatagataatctcanagcaaacttcaatgctcactacctaggttggtttggggggctgctttgctttttccctc

SEQ ID NO: 694 - DNA, 119 bp, Bos taurus
  misc_feature (60)..(60): T OR C
ttatatcaggcacataaaaatgtatagataatctcacagcaaacttcaatgctcactacntaggttggtttggggggctgctttgctttttccctccctcctcctcctacacattcact

SEQ ID NO: 695 - DNA, 119 bp, Bos taurus
  misc_feature (60)..(60): T OR C
gctttttccctccctcctcctcctacacattcactacatgtgtatgtccatttataatanagatatatgtttttcatgcttttaaattctatgtagatggacccatagtctaggtatcc

SEQ ID NO: 696 - DNA, 119 bp, Bos taurus
  misc_feature (60)..(60): G OR A
ctttttccctccctcctcctcctacacattcactacatgtgtatgtccatttataatacngatatatgtttttcatgcttttaaattctatgtagatggacccatagtctaggtatcct

SEQ ID NO: 697 - DNA, 119 bp, Bos taurus
  misc_feature (60)..(60): C OR A
atgtttttcatgcttttaaattctatgtagatggacccatagtctaggtatcctgatatnacctggttttttttagcccaacactttatgagttttatccacgttgatatagacactct

SEQ ID NO: 698 - DNA, 119 bp, Bos taurus
  misc_feature (60)..(60): C OR T
atattcaaatcatgtagggtactgaataaataatggaaacctccctttacctacttctcnatctcaaaatttaccaccatgaccacctatgtgtattctgccagccctttttctaggca

SEQ ID NO: 699 - DNA, 119 bp, Bos taurus
  misc_feature (60)..(60): C OR G
taactccagtgtctgctggataaaagccacactccccattctggcactcagggcctcgtntctcagttccccagtcctcaggcagcctgtacagggtcttactgaaccagcgttcctct

SEQ ID NO: 700 - DNA, 119 bp, Bos taurus
  misc_feature (60)..(60): G OR C
aagccacactccccattctggcactcagggcctcgtgtctcagttccccagtcctcaggnagcctgtacagggtcttactgaaccagcgttcctctgagagcccacgtgttccagacct

SEQ ID NO: 701 - DNA, 119 bp, Bos taurus
  misc_feature (60)..(60): C OR T
ctggcactcagggcctcgtgtctcagttccccagtcctcaggcagcctgtacagggtctnactgaaccagcgttcctctgagagcccacgtgttccagacctcggtcagagagagcagg

SEQ ID NO: 702 - DNA, 119 bp, Bos taurus
  misc_feature (60)..(60): A OR G
gcctcgtgtctcagttccccagtcctcaggcagcctgtacagggtcttactgaaccagcnttcctctgagagcccacgtgttccagacctcggtcagagagagcaggacaggggtcaat

SEQ ID NO: 703 - DNA, 119 bp, Bos taurus
  misc_feature (60)..(60): A OR G
gtgttccagacctcggtcagagagagcaggacaggggtcaatcctgagtctcatgccccncccgcactagccaagttagcccgctcatgggtgccacttccacttctgagaggctggct

SEQ ID NO: 704 - DNA, 119 bp, Bos taurus
  misc_feature (60)..(60): A OR G
gagcaggacaggggtcaatcctgagtctcatgccccgcccgcactagccaagttagcccnctcatgggtgccacttccacttctgagaggctggctccagtgtcctccctgacttgaga

SEQ ID NO: 705 - DNA, 119 bp, Bos taurus
  misc_feature (60)..(60): C OR A
ctgcagacagtggggtctggaggcctcacttggtagacaaaggatggtgcattataattntgttttcacttagcagttcacttctctgccacactcgctaactaagatccaggaagaaa

SEQ ID NO: 706 - DNA, 119 bp, Bos taurus
  misc_feature (60)..(60): T OR A
atgccaattggattcatgagcttcgcctctcccactaatggggtcaggctctggcggccnttattgcacagaggctccgggagaagcaaacacacaggcgagacttcaccgccacttat

SEQ ID NO: 707 - DNA, 119 bp, Bos taurus
  misc_feature (60)..(60): T OR C
agggctgcgcagaacccacattccctaatgtgggggatcgacatacctccctcagcatcnaggcctgaagcttctgaaccctgactttaaggccgggcacactgaaacttgtcggagag

SEQ ID NO: 708 - DNA, 119 bp, Bos taurus
  misc_feature (60)..(60): T OR G
tcgacatacctccctcagcatccaggcctgaagcttctgaaccctgactttaaggccggncacactgaaacttgtcggagagacctagccacagggagttcctagtctgagagaaaccc

SEQ ID NO: 709 - DNA, 119 bp, Bos taurus
  misc_feature (60)..(60): A OR G
gaagtaagtatattattattcctattttatggtgaaggtcagaaaggttagatgtctgantttacacactgttaagtgatggggtcagaattcaaacccggagtcatcttagacactac

SEQ ID NO: 710 - DNA, 119 bp, Bos taurus
  misc_feature (60)..(60): T OR C
gtatattattattcctattttatggtgaaggtcagaaaggttagatgtctgagtttacanactgttaagtgatggggtcagaattcaaacccggagtcatcttagacactacactttgc

SEQ ID NO: 711 - DNA, 119 bp, Bos taurus
  misc_feature (60)..(60): CT OR C
agtgatggggtcagaattcaaacccggagtcatcttagacactacactttgcttctactntttttttttcccctaataaaagtcttttttcttcaattaatttgcttctcttgacttct

SEQ ID NO: 712 - DNA, 119 bp, Bos taurus
  misc_feature (60)..(60): G OR C
gaagtcccagggccataggcctgggccaagaagcacctttgatggtgggtgaggggtctnatgaaaagggcaagaaaggaactgttttcaatcattctcaggacgcaagaatttctggt

SEQ ID NO: 713 - DNA, 119 bp, Bos taurus
  misc_feature (60)..(60): C OR T
caagaagcacctttgatggtgggtgaggggtctcatgaaaagggcaagaaaggaactgtnttcaatcattctcaggacgcaagaatttctggtactgggccacctataccttcaggccc

SEQ ID NO: 714 - DNA, 119 bp, Bos taurus
  misc_feature (60)..(60): T OR C
gtgggtgaggggtctcatgaaaagggcaagaaaggaactgttttcaatcattctcaggangcaagaatttctggtactgggccacctataccttcaggccctctcagaaatccttggga

SEQ ID NO: 715 - DNA, 119 bp, Bos taurus
  misc_feature (60)..(60): G OR A
ccacctataccttcaggccctctcagaaatccttgggatattattgacaggcaacaaacnccgccaaattgtctgccatgcctacacaatgtgtgggcacagagggttatggcttcatt

SEQ ID NO: 716 - DNA, 119 bp, Bos taurus
  misc_feature (60)..(60): G OR A
aattgtctgccatgcctacacaatgtgtgggcacagagggttatggcttcatttgctccntctataatccatgtgtagagaccctcttttccgaacccaaagtgttctgtctaggtcaa

SEQ ID NO: 717 - DNA, 119 bp, Bos taurus
  misc_feature (60)..(60): C OR G
caatggatgaatgcattaatgtaatctcaaagattcgcagtgaaactaagccattgccgntgaaagggtccaagcccagggtgcaaagattattgccccaattccacactccgggctac

SEQ ID NO: 718 - DNA, 119 bp, Bos taurus
  misc_feature (60)..(60): A OR C
taatgtaatctcaaagattcgcagtgaaactaagccattgccggtgaaagggtccaagcncagggtgcaaagattattgccccaattccacactccgggctacttgtaccaggaaccac

SEQ ID NO: 719 - DNA, 119 bp, Bos taurus
  misc_feature (60)..(60): C OR T
cgggctacttgtaccaggaaccaccctgccccctccactggaacgaccacgctgcgcatnccagggaggctcgcctcctctggcggcccagcttccctttctcctcgcgtctttctgtc

SEQ ID NO: 720 - DNA, 119 bp, Bos taurus
  misc_feature (60)..(60): C OR G
tcgggattttctctcccttcttcctgccgccctctcctctaccctgtgacccctgccccntccccgctgtgcccccaccacactctgtcaaactggggccaggagaccagcgctaaggt

SEQ ID NO: 721 - DNA, 119 bp, Bos taurus
  misc_feature (60)..(60): T OR G
aaggtcactgggtttcaacctgcagaatgggaagccatctcccctgtctacaggtgaatnctgttactctgcctccagccatggactgcttgctcttgtgggaaagtggaaagcagctg

SEQ ID NO: 722 - DNA, 119 bp, Bos taurus
  misc_feature (60)..(60): G OR A
caggtgaatgctgttactctgcctccagccatggactgcttgctcttgtgggaaagtggnaagcagctgaccagggaccggggatgctagtcaaccagtaattctcagtgtaacctaga

SEQ ID NO: 723 - DNA, 119 bp, Bos taurus
  misc_feature (60)..(60): G OR A
agtccctaaactatcaggttgggattcaccaaggagattcctgaaaaggagagaaagtancgtggccttgcctaaacctccacatttgcccttataacatctctgccttcactggcctg

SEQ ID NO: 724 - DNA, 119 bp, Bos taurus
  misc_feature (60)..(60): T OR G
ctaaactatcaggttgggattcaccaaggagattcctgaaaaggagagaaagtaacgtgnccttgcctaaacctccacatttgcccttataacatctctgccttcactggcctgggcgt

SEQ ID NO: 725 - DNA, 119 bp, Bos taurus
  misc_feature (60)..(60): C OR CT
aaaaggagagaaagtaacgtggccttgcctaaacctccacatttgcccttataacatctntgccttcactggcctgggcgtggagtgattgcatgtttcttaaggacccactgtgtgcc

SEQ ID NO: 726 - DNA, 119 bp, Bos taurus
  misc_feature (60)..(60): T OR C
tctgccttcactggcctgggcgtggagtgattgcatgtttcttaaggacccactgtgtgncaagcacgcatatatcacagccccactctcagagcgctcgtggcataatagagcagcgt

SEQ ID NO: 727 - DNA, 119 bp, Bos taurus
  misc_feature (60)..(60): A OR G
cactggcctgggcgtggagtgattgcatgtttcttaaggacccactgtgtgccaagcacncatatatcacagccccactctcagagcgctcgtggcataatagagcagcgtggcagggc

SEQ ID NO: 728 - DNA, 119 bp, Bos taurus
  misc_feature (60)..(60): T OR C
gggggtgaagcttcatgccaggctgtactgcacaatgagagagcgagggccaggttctgnctgcactcccccggtcagcagcctcctcagcgcaggacacgacctgctcagcctctgac

SEQ ID NO: 729 - DNA, 119 bp, Bos taurus
  misc_feature (60)..(60): G OR A
tatcctgtatgggaacaggtggggaagctgtctgcaggcaagaaatgggcagactgaggncatggagcacagccttctctcccctccacctcttgctggcacaagcaaactgaatgcag

SEQ ID NO: 730 - DNA, 119 bp, Bos taurus
  misc_feature (60)..(60): T OR C
ctataatagttaatgggtattaaatatttaccatcatgcactgtgctaagtgccccacangcattatctaatttcatcctcaaacaactttggaaatacgtgctattaataatctcatt

SEQ ID NO: 731 - DNA, 119 bp, Bos taurus
  misc_feature (60)..(60): T OR C
actgtgctaagtgccccacacgcattatctaatttcatcctcaaacaactttggaaatangtgctattaataatctcattttactgatgaggattctacgtaacatgcctaaggtcaca

SEQ ID NO: 732 - DNA, 119 bp, Bos taurus
  misc_feature (60)..(60): A OR C
ctcaaacaactttggaaatacgtgctattaataatctcattttactgatgaggattctangtaacatgcctaaggtcacacagctatttagtgagggccctggggatggaattcacagc

SEQ ID NO: 733 - DNA, 119 bp, Bos taurus
  misc_feature (60)..(60): C OR A
gttctatatctgttggactaagtggagaagggctctgaattccatgctagggagcttganctttacaccatgggcaatggggagacactggacggttgtaagcagggagagacgggtca

SEQ ID NO: 734 - DNA, 119 bp, Bos taurus
  misc_feature (60)..(60): A OR G
gagcaagaggccatttttctggcaaatcccaattcctccttgtattttttttaagtcttntcttatctcttagggcaagacttatacgcacagtacatcatgcaaaatgctggactgga

SEQ ID NO: 735 - DNA, 119 bp, Bos taurus
  misc_feature (60)..(60): C OR T
agcaagaggccatttttctggcaaatcccaattcctccttgtattttttttaagtcttgncttatctcttagggcaagacttatacgcacagtacatcatgcaaaatgctggactggat

SEQ ID NO: 736 - DNA, 119 bp, Bos taurus
  misc_feature (60)..(60): T OR C
ggatgaagcataagctggaaccaagattgcagggagaaatatcagtaacctcagatatgnggataagtaacctcagatatgcggataacaccacccttatggcagaaagcaaagaggca

SEQ ID NO: 737 - DNA, 119 bp, Bos taurus
  misc_feature (60)..(60): C OR T
agagcctcttgatgaaggtgaaagaggagaatgaaaaagctggctcaaaactcaacattnggaaaacaaagaccatggcatcgggtcccatcacttcacggcaaatagatagggaaaca

SEQ ID NO: 738 - DNA, 119 bp, Bos taurus
  misc_feature (60)..(60): T OR C
gaaggtgaaagaggagaatgaaaaagctggctcaaaactcaacatttggaaaacaaagancatggcatcgggtcccatcacttcacggcaaatagatagggaaacaatggaaacagtga

SEQ ID NO: 739 - DNA, 119 bp, Bos taurus
  misc_feature (60)..(60): A OR G
gaggagaatgaaaaagctggctcaaaactcaacatttggaaaacaaagaccatggcatcnggtcccatcacttcacggcaaatagatagggaaacaatggaaacagtgacagactttat

SEQ ID NO: 740 - DNA, 119 bp, Bos taurus
  misc_feature (60)..(60): G OR A
actcaacatttggaaaacaaagaccatggcatcgggtcccatcacttcacggcaaatagntagggaaacaatggaaacagtgacagactttattttcttgggttccaaaatcattgcag

SEQ ID NO: 741 - DNA, 119 bp, Bos taurus
  misc_feature (60)..(60): T OR A
aagaaaagctatgacaaacttagccagaggcattattttgctgacaaaggtctgtctagncaaagctatggttttaccagtaatcatatatggatgtgagagttggactataaagaaag

SEQ ID NO: 742 - DNA, 119 bp, Bos taurus
  misc_feature (60)..(60): G OR A
gacaaagctatggttttaccagtaatcatatatggatgtgagagttggactataaagaangctgagtgccaaaaaattgatccttttgaactgtggtattggagaagactcttaagagt

SEQ ID NO: 743 - DNA, 119 bp, Bos taurus
  misc_feature (60)..(60): G OR A
ggttttaccagtaatcatatatggatgtgagagttggactataaagaaagctgagtgccnaaaaattgatccttttgaactgtggtattggagaagactcttaagagtcccttggacac

SEQ ID NO: 744 - DNA, 119 bp, Bos taurus
  misc_feature (60)..(60): G OR A
tttaccagtaatcatatatggatgtgagagttggactataaagaaagctgagtgccaaanaattgatccttttgaactgtggtattggagaagactcttaagagtcccttggacaccaa

SEQ ID NO: 745 - DNA, 119 bp, Bos taurus
  misc_feature (60)..(60): T OR TGCTG
gaagctccaatactttgaccacttgatgtgaagaactgactcactggaaaagaccctgangctggctgggaaagattgaaagcaggaggagaaggggatgacagaggatgaaatggttg

SEQ ID NO: 746 - DNA, 119 bp, Bos taurus
  misc_feature (60)..(60): A OR G
tcactgacttgatggacatgagtttgagcaagctctgggagttggtgatggatagggaancctagcgtgctgcagtcatggagtctcaaagagtcagacaggactgagcaactgaacta

SEQ ID NO: 747 - DNA, 119 bp, Bos taurus
  misc_feature (60)..(60): G OR C
cccttctctgggcagctcatgatatcactggcttttccagcaagtggggtggggagttanaggaaatggcctggggcatcagataagctgaagaaagaggccgagtagctggaaagacg

SEQ ID NO: 748 - DNA, 119 bp, Bos taurus
  misc_feature (60)..(60): T OR C
gggtcccaagatgctgctctgcgtcaccttgcttctcctcctggggctgtctgcatgcantgtggcaggtgacaaggaactggcaatcaatgctgaagttggctcctgggtggctgtga

SEQ ID NO: 749 - DNA, 119 bp, Bos taurus
  misc_feature (60)..(60): G OR A
ccttgcttctcctcctggggctgtctgcatgcactgtggcaggtgacaaggaactggcantcaatgctgaagttggctcctgggtggctgtgaccctggaggtaggtacctttggggaa

SEQ ID NO: 750 - DNA, 119 bp, Bos taurus
  misc_feature (60)..(60): C OR T
aggtaggtacctttggggaaagcagggcatgatagcaagagagcaggctgtacactgcanggtagggtgtctgcctcccctctctgggcatgttcaaggctgttgcccctggcctgata

SEQ ID NO: 751 - DNA, 119 bp, Bos taurus
  misc_feature (60)..(60): C OR T
tcaggcttccctgtccttcaccatctcccagagcttgctcaaactcatgttcatcaagtnggtgatgccatccaaccatctcatcctctgtccccttctcctcctgccttcaatctttc

SEQ ID NO: 752 - DNA, 119 bp, Bos taurus
  misc_feature (60)..(60): GTCA OR G
gagcttgctcaaactcatgttcatcaagttggtgatgccatccaaccatctcatcctctnccttctcctcctgccttcaatctttcccagcatcagggtcttttccagtgagtcagttc

SEQ ID NO: 753 - DNA, 119 bp, Bos taurus
  misc_feature (60)..(60): T OR C
ctcatcctctgtccccttctcctcctgccttcaatctttcccagcatcagggtcttttcnagtgagtcagttcttcacatcaggtaatcaaagtattggagcttcagcttcagcatcag

SEQ ID NO: 754 - DNA, 119 bp, Bos taurus
  misc_feature (60)..(60): G OR A
ccttcaatctttcccagcatcagggtcttttccagtgagtcagttcttcacatcaggtantcaaagtattggagcttcagcttcagcatcagtccttccaatgaatattcaggactgat

SEQ ID NO: 755 - DNA, 119 bp, Bos taurus
  misc_feature (60)..(60): G OR C
atattcaggactgatttcctttaggattgactgatttggtctccttgctgtccaagggantctcaagagtcttcttcaataccacagttcaaaaggatcaattttttggcactcagcct

SEQ ID NO: 756 - DNA, 119 bp, Bos taurus
  misc_feature (60)..(60): C OR T
tcctttaggattgactgatttggtctccttgctgtccaagggactctcaagagtcttctncaataccacagttcaaaaggatcaattttttggcactcagccttctttatagtccaact

SEQ ID NO: 757 - DNA, 119 bp, Bos taurus
  misc_feature (60)..(60): G OR A
ccaagggactctcaagagtcttcttcaataccacagttcaaaaggatcaattttttggcnctcagccttctttatagtccaactctcacatccatacatgactactggaaaaaccatag

SEQ ID NO: 758 - DNA, 119 bp, Bos taurus
  misc_feature (60)..(60): C OR T
gtccaactctcacatccatacatgactactggaaaaaccatagctttgactagacagaantttgtcagcaaaataatgtctctgcttcttaatatgctgagtttgtcatagcttttctt

SEQ ID NO: 759 - DNA, 119 bp, Bos taurus
  misc_feature (60)..(60): C OR G
agacagaattttgtcagcaaaataatgtctctgcttcttaatatgctgagtttgtcatancttttcttccaaggagcaagcatcttttaatttcatggctgcggtcaccatctgcagtg

SEQ ID NO: 760 - DNA, 119 bp, Bos taurus
  misc_feature (60)..(60): G OR A
tgtcactgttatctatttgccatgaagtgatgggacccaatgccatgaactttgttttcnaatgttgagttttgagccagctttttcattctcctctttcaccttcatcaagaggctct

SEQ ID NO: 761 - DNA, 119 bp, Bos taurus
  misc_feature (60)..(60): T OR C
atacaggtttctcaggaggcagataaggtggcctggtattcccatctctttaagaatttnccacagtttgttgtgatccacacagtcaaaggttttagcgtagtcaacgaagcagaagt

SEQ ID NO: 762 - DNA, 119 bp, Bos taurus
  misc_feature (60)..(60): G OR A
aaatccagcttaaacatctggaagttctcggttcacatactgttgaagcctaacttgganaattttgagcattactttgctagcatgtgacattagtgtagttgtgcggtaatttgaac

SEQ ID NO: 763 - DNA, 119 bp, Bos taurus
  misc_feature (60)..(60): T OR C
ttagtgtagttgtgcggtaatttgaacattctttggaattgcctttcttgggattggaangaaaactttttccagtcctatggcaactgctgagtttttccaaattttctggcatattg

SEQ ID NO: 764 - DNA, 119 bp, Bos taurus
  misc_feature (60)..(60): G OR A
tggaacgaaaactttttccagtcctatggcaactgctgagtttttccaaattttctggcntattgagtgcagcactttcacagcatcatcttttaggatttgaaatatctcaactggaa

SEQ ID NO: 765 - DNA, 119 bp, Bos taurus
  misc_feature (60)..(60): G OR C
agatgtctagtctttcccattctattgttttcctctatttgcattgatcacttaagaagnctttcttatctctccttgctattctctggtctctgcattcagatggatatatctttcct

SEQ ID NO: 766 - DNA, 119 bp, Bos taurus
  misc_feature (60)..(60): C OR T
ttttcttggggatggttttgatcaccgcctcctatacaatgtgaacctccgtccatagtncttcaggcactctatcagatctaatcctttgaatctatttgtcacgtcacctgtataat

SEQ ID NO: 767 - DNA, 119 bp, Bos taurus
  misc_feature (60)..(60): A OR AGTAT
aattgtaagggatttgatttatctcatacctgaatggcctagtgatatatggaattaacngtatgtgtgttcatgcttcatttaacaaacttatttaatatatacatgttcctgacact

SEQ ID NO: 768 - DNA, 119 bp, Bos taurus
  misc_feature (60)..(60): C OR G
caatgtcttattctgtgtatctgtgcctcggaggacattgtccagcacatcctttcaagngggtcaacttggagtgggatcactgttttgtctcatgtgtagaaacttattccctgaca

SEQ ID NO: 769 - DNA, 119 bp, Bos taurus
  misc_feature (60)..(60): G OR A
aatctcacagctagagacacagcgtctgtgccccaccccatctagtcagagagacaaagntcctggcctcggaggttcccatcagacaggggcacgaggccagtggactagagacacac

SEQ ID NO: 770 - DNA, 119 bp, Bos taurus
  misc_feature (60)..(60): A OR G
agcaggctgaggagataaaggttcaagtctcagcttctccaccgactctgtgtgacctgnggacatcagtttcctcacttgtaaaatgggagagggggggaaaaagatggcccccaagg

SEQ ID NO: 771 - DNA, 119 bp, Bos taurus
  misc_feature (60)..(60): G OR A
ccgactctgtgtgacctggggacatcagtttcctcacttgtaaaatgggagagggggggnaaaagatggcccccaaggttttttttccagctctgacttggagtctgtgcccatcaggg

SEQ ID NO: 772 - DNA, 119 bp, Bos taurus
  misc_feature (60)..(60): G OR A
cgactctgtgtgacctggggacatcagtttcctcacttgtaaaatgggagaggggggganaaagatggcccccaaggttttttttccagctctgacttggagtctgtgcccatcagggg

SEQ ID NO: 773 - DNA, 119 bp, Bos taurus
  misc_feature (60)..(60): C OR T
gtaatagggaccactctacacggttacgtgggaaacatcatcgatttcaaccagtgcctngtggggctttccatgctgcagctgcatctgaagcttcttcttggctcactctcatttaa

SEQ ID NO: 774 - DNA, 119 bp, Bos taurus
  misc_feature (60)..(60): C OR T
ccagtgccttgtggggctttccatgctgcagctgcatctgaagcttcttcttggctcacnctcatttaacatccctctacttcccaagacacagaatttgggatataggaaccatctca

SEQ ID NO: 775 - DNA, 119 bp, Bos taurus
  misc_feature (60)..(60): A OR C
ccatctcatctgaatgaggaatgatgcctctgttagggatgatctgattttctcccttgntctcaggactcccagttagccctggagctggggatttgagttaaaggggtaagaagagc

SEQ ID NO: 776 - DNA, 119 bp, Bos taurus
  misc_feature (60)..(60): A OR G
gatctgattttctcccttgctctcaggactcccagttagccctggagctggggatttganttaaaggggtaagaagagcttctggtcagggcaagagtccttcctgagtaaaagacacc

SEQ ID NO: 777 - DNA, 119 bp, Bos taurus
  misc_feature (60)..(60): A OR G
agccctggagctggggatttgagttaaaggggtaagaagagcttctggtcagggcaagantccttcctgagtaaaagacacctcccaacgggccccagaaacctaatgggtgggagaaa

SEQ ID NO: 778 - DNA, 119 bp, Bos taurus
  misc_feature (60)..(60): T OR C
gaagatcctctgggggaagatcccctggagaaggaaacggcaacccactccagtactctngcctggaaaatcccatagatggaggagcctaggaggctgcagtccatggggtcacaaag

SEQ ID NO: 779 - DNA, 119 bp, Bos taurus
  misc_feature (60)..(60): A OR G
ggaaaatcccatagatggaggagcctaggaggctgcagtccatggggtcacaaagagtcngacacgactgagtgacttcactttgagctaaaggatgaaaatgtctttgcctgagggag

SEQ ID NO: 780 - DNA, 119 bp, Bos taurus
  misc_feature (60)..(60): A OR G
ctaatcacaaacctgtgtccccatctgtagatggatatttcacatggtggttgtgaagcntaaatgagattatgcctggagtgtctgagcctttgagttagatgacctgggttcgaggc

SEQ ID NO: 781 - DNA, 119 bp, Bos taurus
  misc_feature (60)..(60): A OR T
ttcagactgttttgttgtcagcctgccctcaactctaaggaactctctgcttcttcaggnagatgtcatccccagcatcctgcttcaactccggaacgtgaagaaggggaaagccagcc

SEQ ID NO: 782 - DNA, 119 bp, Bos taurus
  misc_feature (60)..(60): G OR A
taaggaactctctgcttcttcaggtagatgtcatccccagcatcctgcttcaactccggnacgtgaagaaggggaaagccagccagttctttgggctaatggggaagcaggtaggaggt

SEQ ID NO: 783 - DNA, 119 bp, Bos taurus
  misc_feature (60)..(60): A OR C
caggtagatgtcatccccagcatcctgcttcaactccggaacgtgaagaaggggaaagcnagccagttctttgggctaatggggaagcaggtaggaggtgagtgacagatgtgggcgga

SEQ ID NO: 784 - DNA, 119 bp, Bos taurus
  misc_feature (60)..(60): C OR T
ttgggctaatggggaagcaggtaggaggtgagtgacagatgtgggcggacacccaggganaaaggcttatgggtgggttctgttaccccaaacagaggccctaacttaaagcccaggac

SEQ ID NO: 785 - DNA, 119 bp, Bos taurus
  misc_feature (60)..(60): C OR A
tggggaagcaggtaggaggtgagtgacagatgtgggcggacacccagggataaaggcttntgggtgggttctgttaccccaaacagaggccctaacttaaagcccaggactcagcatcc

SEQ ID NO: 786 - DNA, 119 bp, Bos taurus
  misc_feature (60)..(60): A OR G
taccccaaacagaggccctaacttaaagcccaggactcagcatcccttactccagtccantagtggtcactcacgctatggaatgggatacagtgtccatatcaattgttatccaaatc

SEQ ID NO: 787 - DNA, 119 bp, Bos taurus
  misc_feature (60)..(60): A OR G
aaacagaggccctaacttaaagcccaggactcagcatcccttactccagtccagtagtgntcactcacgctatggaatgggatacagtgtccatatcaattgttatccaaatcaggaca

SEQ ID NO: 788 - DNA, 119 bp, Bos taurus
  misc_feature (60)..(60): T OR C
cagcatcccttactccagtccagtagtggtcactcacgctatggaatgggatacagtgtncatatcaattgttatccaaatcaggacatttcagagagtgttaattatgccaggcaaca

SEQ ID NO: 789 - DNA, 119 bp, Bos taurus
  misc_feature (60)..(60): T OR TCTGA
cactgtccatttggaggacattttggcaatatccatcaaagttggcaacatgcatacctnctgactgataggttcgcttcaggaaatccatcaaatagaaatgctattgctacataagc

SEQ ID NO: 790 - DNA, 119 bp, Bos taurus
  misc_feature (60)..(60): G OR A
aaggtgaattcttaaccactggaccaccagggaagtctggcacatccatagcctttgtgnccattgaagtggatgaggcagatctgtggaaagataaagatttccaagatattggctaa

SEQ ID NO: 791 - DNA, 119 bp, Bos taurus
  misc_feature (60)..(60): T OR C
cacacatgaaaacatagggagagttccaggataaatatcaaactgtcaccagtgttttanatctaggatagaagtgaaggtgagggagagcaaaagttaagggggacttttactacact

SEQ ID NO: 792 - DNA, 119 bp, Bos taurus
  misc_feature (60)..(60): C OR T
gagggagagcaaaagttaagggggacttttactacactaaactctaccttaccacttganagtgggcatttattcagaaactgcttttgtaactttaaaaaacacatttaaaaatagag

SEQ ID NO: 793 - DNA, 119 bp, Bos taurus
  misc_feature (60)..(60): A OR AAT
ctttaaaaaacacatttaaaaatagagaaaatcttcctgctacatgcaccataaaataanatagagaaaattaacttgtgaccaaactttggcctcagtttcttccacaaaatgagttt

SEQ ID NO: 794 - DNA, 119 bp, Bos taurus
  misc_feature (60)..(60): C OR CCACAAAATGAGTTTTGG
gcaccataaaataaaatagagaaaattaacttgtgaccaaactttggcctcagtttcttncacaaaatgagttttggcctgggccatttcagagatctctttcaaatccagaaacccag

SEQ ID NO: 795 - DNA, 119 bp, Bos taurus
  misc_feature (60)..(60): T OR C
cctttatcaccagcagggaaactaaggttcacataggctaagtactttcccaaggacgcntgactagcaccaggcagactccacagtatgcacacttcccactaccctaaactgcctcc

SEQ ID NO: 796 - DNA, 119 bp, Bos taurus
  misc_feature (60)..(60): C OR T
ggcagactccacagtatgcacacttcccactaccctaaactgcctcctacccagatgtanctggtgtggcccagtcacaccaggacaacagggggagaccagagtccccaaagacccac

SEQ ID NO: 797 - DNA, 119 bp, Bos taurus
  misc_feature (60)..(60): A OR G
ccacagtatgcacacttcccactaccctaaactgcctcctacccagatgtatctggtgtngcccagtcacaccaggacaacagggggagaccagagtccccaaagacccaccaaaggca

SEQ ID NO: 798 - DNA, 119 bp, Bos taurus
  misc_feature (60)..(60): A OR G
ctaaactgcctcctacccagatgtatctggtgtggcccagtcacaccaggacaacagggngagaccagagtccccaaagacccaccaaaggcatggatcccaaagagccatgagctgtt

SEQ ID NO: 799 - DNA, 119 bp, Bos taurus
  misc_feature (60)..(60): T OR A
aaactgcctcctacccagatgtatctggtgtggcccagtcacaccaggacaacagggggngaccagagtccccaaagacccaccaaaggcatggatcccaaagagccatgagctgtttc

SEQ ID NO: 800 - DNA, 119 bp, Bos taurus
  misc_feature (60)..(60): G OR A
atttcttctctttcccctaggaatacctcccatccagtcagagggaacaggtaagtgttntccacaccttccccccacatgctccctggggaagaccgagttgtcagggaggtgaggga

SEQ ID NO: 801 - DNA, 119 bp, Bos taurus
  misc_feature (60)..(60): C OR G
gcataggcttcctggttggttccttggagaggtggtctgtgtgcgccgagggagcagctncaaggatgaggagatggcgatgaatccagttctgccaaaggttcaaaattaccatgggt

SEQ ID NO: 802 - DNA, 119 bp, Bos taurus
  misc_feature (60)..(60): C OR T
caatccagaaggaccttaaaactgaaaaccctaaccctgtcttcatggcctgggcatggngccctccttggcatactgccctgcactgttctgctgggtcacatggctggtgtcctgag

SEQ ID NO: 803 - DNA, 119 bp, Bos taurus
  misc_feature (60)..(60): A OR G
aaactgaaaaccctaaccctgtcttcatggcctgggcatggtgccctccttggcatactnccctgcactgttctgctgggtcacatggctggtgtcctgagagtaaaaaccaccccacg

SEQ ID NO: 804 - DNA, 119 bp, Bos taurus
  misc_feature (60)..(60): T OR TTGGTTCCCAAGACAG
acctcagttcctagagaaattactctattcctgagtccccagttctggtcctgtcttttntggttcccaagacagtcttcctctgcttcactttctctttttcttctctgtctcctctc

SEQ ID NO: 805 - DNA, 119 bp, Bos taurus
  misc_feature (60)..(60): C OR T
cagttctggtcctgtcttttttggttcccaagacagtcttcctctgcttcactttctctntttcttctctgtctcctctcttcatcacaagggtatcagcgagaaccagtggtccaggg

SEQ ID NO: 806 - DNA, 119 bp, Bos taurus
  misc_feature (60)..(60): C OR T
ggtatcagcgagaaccagtggtccaggggcgaggaggagcaactacagaaggtaggtganagcttctccagcagcacacacaggcacattctgctccccactctcagggccagctgaag

SEQ ID NO: 807 - DNA, 119 bp, Bos taurus
  misc_feature (60)..(60): C OR G
gaaccagtggtccaggggcgaggaggagcaactacagaaggtaggtgatagcttctccancagcacacacaggcacattctgctccccactctcagggccagctgaagccaccgagaat

SEQ ID NO: 808 - DNA, 119 bp, Bos taurus
  misc_feature (60)..(60): A OR G
aggtaggtgatagcttctccagcagcacacacaggcacattctgctccccactctcaggnccagctgaagccaccgagaatgctgtgtggttctccagaccctggaacacgccccttat

SEQ ID NO: 809 - DNA, 119 bp, Bos taurus
  misc_feature (60)..(60): A OR ACC
cttctccagcagcacacacaggcacattctgctccccactctcagggccagctgaagccnccgagaatgctgtgtggttctccagaccctggaacacgccccttattccctaattgtta

SEQ ID NO: 810 - DNA, 119 bp, Bos taurus
  misc_feature (60)..(60): T OR C
tcttaggtattttctgtcttcaacaacctaaataaaggcaatatcactctgggaggggtnatcagctgacgctttctggcctgggccacaaccacaaaggcgttcaaggggatgttcaa

SEQ ID NO: 811 - DNA, 119 bp, Bos taurus
  misc_feature (60)..(60): G OR A
ttgagccatttcatttgaagggccatggaagtcaccccagtgccaacatggaaatgcacntccctccattcaaagcgctggcaggaaccttagaaatcatcatttttcagggatggaaa

SEQ ID NO: 812 - DNA, 119 bp, Bos taurus
  misc_feature (60)..(60): G OR T
tcatttgaagggccatggaagtcaccccagtgccaacatggaaatgcacatccctccatncaaagcgctggcaggaaccttagaaatcatcatttttcagggatggaaacaatttgccc

SEQ ID NO: 813 - DNA, 119 bp, Bos taurus
  misc_feature (60)..(60): G OR A
gtcacacggtgaactgttaacagagccaggactaaacaccgtctcctgctccctagtccnggcacttcatgggaccctcagagggccattggaattttaagtgctggattaccactccc

SEQ ID NO: 814 - DNA, 119 bp, Bos taurus
  misc_feature (60)..(60): A OR G
agagaggattagtgagggttggctcatccactatctagaggcatttatgattgaaaaacngaagctctgccagattaacctggaatttgattgccctaaataactacagttggagaaac

SEQ ID NO: 815 - DNA, 119 bp, Bos taurus
  misc_feature (60)..(60): C OR T
ggctcccctaaccactgcttatgtgcagcactcttcccaggcaaagaggctgagaaccangggtcagagtgagagcccccagccccgcctcccagaggacgaaacccctctccttacct

SEQ ID NO: 816 - DNA, 119 bp, Bos taurus
  misc_feature (60)..(60): T OR G
aggacgaaacccctctccttacctggacgtggcagccaaccagccgaccaggccagcccnctgctccaggtcttctgtgctcgtgtgtccctcccaccacgctgaactcactgggctcc

SEQ ID NO: 817 - DNA, 119 bp, Bos taurus
  misc_feature (60)..(60): T OR C
aacccctctccttacctggacgtggcagccaaccagccgaccaggccagcccgctgctcnaggtcttctgtgctcgtgtgtccctcccaccacgctgaactcactgggctcctcccagg

SEQ ID NO: 818 - DNA, 119 bp, Bos taurus
  misc_feature (60)..(60): G OR A
acccctctccttacctggacgtggcagccaaccagccgaccaggccagcccgctgctccnggtcttctgtgctcgtgtgtccctcccaccacgctgaactcactgggctcctcccagga

SEQ ID NO: 819 - DNA, 119 bp, Bos taurus
  misc_feature (60)..(60): T OR G
cccctctccttacctggacgtggcagccaaccagccgaccaggccagcccgctgctccangtcttctgtgctcgtgtgtccctcccaccacgctgaactcactgggctcctcccaggac

SEQ ID NO: 820 - DNA, 119 bp, Bos taurus
  misc_feature (60)..(60): G OR A
ccaggactctcggtctcaagcagcagtcgtgcacaagtgtgctgttgtcacctctgaaanctagtgaatgaccccacacactcggacctcactgcatagcaggagtcgtcccactgata

SEQ ID NO: 821 - DNA, 119 bp, Bos taurus
  misc_feature (60)..(60): A OR G
gttgtcacctctgaaaactagtgaatgaccccacacactcggacctcactgcatagcagnagtcgtcccactgataccttcctacccccgtttctctcaatcctgtgcccataacacag

SEQ ID NO: 822 - DNA, 119 bp, Bos taurus
  misc_feature (60)..(60): A OR C
cactgataccttcctacccccgtttctctcaatcctgtgcccataacacagcaagtgttnaataaaccaatgaagagcgaatgatttcttctctggtgcagggcagcggacagggatga

SEQ ID NO: 823 - DNA, 119 bp, Bos taurus
  misc_feature (60)..(60): T OR C
tcaatcctgtgcccataacacagcaagtgttcaataaaccaatgaagagcgaatgatttnttctctggtgcagggcagcggacagggatgacctgggctcaacaccctgcccaggggcc

SEQ ID NO: 824 - DNA, 119 bp, Bos taurus
  misc_feature (60)..(60): C OR G
cacagcaagtgttcaataaaccaatgaagagcgaatgatttcttctctggtgcagggcancggacagggatgacctgggctcaacaccctgcccaggggcctggcgtgtcagcagccag

SEQ ID NO: 825 - DNA, 119 bp, Bos taurus
  misc_feature (60)..(60): A OR G
ctggtgcagggcagcggacagggatgacctgggctcaacaccctgcccaggggcctggcntgtcagcagccaggagacctcttccagagttgtaaaggcaaacactaaatagccccggt

SEQ ID NO: 826 - DNA, 119 bp, Bos taurus
  misc_feature (60)..(60): G OR C
accctgcccaggggcctggcgtgtcagcagccaggagacctcttccagagttgtaaaggnaaacactaaatagccccggtcctctgaggtgggaaaacgggcttgggacacaggtagcc

SEQ ID NO: 827 - DNA, 119 bp, Bos taurus
  misc_feature (60)..(60): C OR T
gggggccactgtcctggcccagctgtggtcctgggaggtggctcactgtgacaaccccangtgacgaagagagggctgaagattttattcccatgtttgaagctggagacattcaagct

SEQ ID NO: 828 - DNA, 119 bp, Bos taurus
  misc_feature (60)..(60): A OR C
agattttattcccatgtttgaagctggagacattcaagcttcggctgaagccactgaccnttcagtgagttccctccttgggctggcactttgctgggccctcaggcgggactgggagc

SEQ ID NO: 829 - DNA, 119 bp, Bos taurus
  misc_feature (60)..(60): A OR G
attcccatgtttgaagctggagacattcaagcttcggctgaagccactgacccttcagtnagttccctccttgggctggcactttgctgggccctcaggcgggactgggagcagggagg

SEQ ID NO: 830 - DNA, 119 bp, Bos taurus
  misc_feature (60)..(60): A OR G
cttgggctggcactttgctgggccctcaggcgggactgggagcagggagggagactggantttgtccttgaagaactaagaacaaagagggcagaagcaatgtactcactcaaggcagc

SEQ ID NO: 831 - DNA, 119 bp, Bos taurus
  misc_feature (60)..(60): AG OR A
gggccctcaggcgggactgggagcagggagggagactggagtttgtccttgaagaactanaacaaagagggcagaagcaatgtactcactcaaggcagcccttgctctccaacaactcg

SEQ ID NO: 832 - DNA, 119 bp, Bos taurus
  misc_feature (60)..(60): A OR G
ccccgtgccaatagagctcagaggtccttggagagggttttcacgaaggactcacgtagnatgcatggtcctcatgctcagcttttgacctgcagccccacgacaaactgctttcgttg

SEQ ID NO: 833 - DNA, 119 bp, Bos taurus
  misc_feature (60)..(60): C OR G
cgtgccaatagagctcagaggtccttggagagggttttcacgaaggactcacgtaggatncatggtcctcatgctcagcttttgacctgcagccccacgacaaactgctttcgttggct

SEQ ID NO: 834 - DNA, 119 bp, Bos taurus
  misc_feature (60)..(60): C OR T
ttgacctgcagccccacgacaaactgctttcgttggctcaggtctacagacggcactgcngcacaccctgctgtgggccagaaacctgggagtgactccccagccctcactgccccctc

SEQ ID NO: 835 - DNA, 119 bp, Bos taurus
  misc_feature (60)..(60): G OR C
tgggccagaaacctgggagtgactccccagccctcactgccccctctaaacacctccccntagttagtcattcttgaaaatatacctaaattcttcttgccgctcttactacacttatt

SEQ ID NO: 836 - DNA, 119 bp, Bos taurus
  misc_feature (60)..(60): A OR G
cctctaaacacctccccctagttagtcattcttgaaaatatacctaaattcttcttgccnctcttactacacttattcacaggaggtactatcttcccttgcctagggggcagcagcct

SEQ ID NO: 837 - DNA, 119 bp, Bos taurus
  misc_feature (60)..(60): T OR C
ccttcaaggccctgtgctccttgatcctcaccccccatcccccctcagccactaccaganattctctcagacccttaggcacaccagaggctcgccagcttttgtgatctgtgcttagg

SEQ ID NO: 838 - DNA, 119 bp, Bos taurus
  misc_feature (60)..(60): C OR T
acccttaggcacaccagaggctcgccagcttttgtgatctgtgcttaggctctccctctnggaacgctgcccacgcccccctccccacccaactggctaacccaatcatctaagcaagc

SEQ ID NO: 839 - DNA, 119 bp, Bos taurus
  misc_feature (60)..(60): T OR C
cagaggctcgccagcttttgtgatctgtgcttaggctctccctcttggaacgctgcccangcccccctccccacccaactggctaacccaatcatctaagcaagctcccttctcagtca

SEQ ID NO: 840 - DNA, 119 bp, Bos taurus
  misc_feature (60)..(60): T OR A
ccctcttggaacgctgcccacgcccccctccccacccaactggctaacccaatcatctangcaagctcccttctcagtcagggcttcctgaataaccaagctcagctagttctctccag

SEQ ID NO: 841 - DNA, 119 bp, Bos taurus
  misc_feature (60)..(60): C OR G
caatcatctaagcaagctcccttctcagtcagggcttcctgaataaccaagctcagctanttctctccagttatgcttacccaacacctgtcctttgcccttcagggcactgatcacaa

SEQ ID NO: 842 - DNA, 119 bp, Bos taurus
  misc_feature (60)..(60): C OR CA
cagggcactgatcacaattaggaatttaatgctcatcttctccaggagaccaagttctanatcttttgtgtacaaatgaatgactgaatatattaggcactctaaaagacaagttacct

SEQ ID NO: 843 - DNA, 119 bp, Bos taurus
  misc_feature (60)..(60): A OR T
gactgaatatattaggcactctaaaagacaagttacctccacaagtattatcgtcaatcngcaccggaaatcccagagccaggatttgagggaaggtccttcactccaagcacatttga

SEQ ID NO: 844 - DNA, 119 bp, Bos taurus
  misc_feature (60)..(60): C OR G
tacagacatgtctcaggcagaaaaataaggaagtcttcacaaggaattcctttttagttnggccatttcagtaagtaccaaagaaacatctaaatgtctgcccccacagcatactggtc

SEQ ID NO: 845 - DNA, 119 bp, Bos taurus
  misc_feature (60)..(60): G OR A
gactaatccagctgtatgtggaggctgtgtaggggaatgatgaaaactacccaggtggantccacttatggaaacttgaaagccaagataaatgggaacttaatccaagaatgggaagc

SEQ ID NO: 846 - DNA, 119 bp, Bos taurus
  misc_feature (60)..(60): C OR A
caggtggaatccacttatggaaacttgaaagccaagataaatgggaacttaatccaagantgggaagccacatgaactccaggggaactggagacctatttgtaacaggatattcacct

SEQ ID NO: 847 - DNA, 119 bp, Bos taurus
  misc_feature (60)..(60): G OR C
ctccaggggaactggagacctatttgtaacaggatattcacctttcctgatccacttctntaaggtggtgactaagaattctaaggctaggcaaggacctgagaggcaacttgactgga

SEQ ID NO: 848 - DNA, 119 bp, Bos taurus
  misc_feature (60)..(60): T OR C
catgaatactgctcccctggagctacacaaggtggaggtcctcctctgggcacatctggnttccaatcacaggcaatcattcaccaaaataaaactacaaggcccaactgtgaaggcag

SEQ ID NO: 849 - DNA, 119 bp, Bos taurus
  misc_feature (60)..(60): A OR G
attaattaacccaagttcagaaatgacttcaaagtctgtttcctaaaccgtatacaaatntgtgcgtatctgcatttatgtacacttttcttttaaaaaacccaatacttttattagat

SEQ ID NO: 850 - DNA, 119 bp, Bos taurus
  misc_feature (60)..(60): A OR G
taacccaagttcagaaatgacttcaaagtctgtttcctaaaccgtatacaaatgtgtgcntatctgcatttatgtacacttttcttttaaaaaacccaatacttttattagattctcaa

SEQ ID NO: 851 - DNA, 119 bp, Bos taurus
  misc_feature (60)..(60): T OR A
tcctaaaccgtatacaaatgtgtgcgtatctgcatttatgtacacttttcttttaaaaancccaatacttttattagattctcaaaaggtcagaaggaaaggtcctttactctcaagtc

SEQ ID NO: 852 - DNA, 119 bp, Bos taurus
  misc_feature (60)..(60): T OR A
cttttaaaaaacccaatacttttattagattctcaaaaggtcagaaggaaaggtcctttnctctcaagtccttttttcacttacacactgctatgtatctgtatgtatatctaaccatc

SEQ ID NO: 853 - DNA, 119 bp, Bos taurus
  misc_feature (60)..(60): T OR A
cccaaaatagtcagtggtttaaagtacttccagggacttccctggcagtccaattgttangactgtgcacatctactgtaggggtcatgggtttgatccctagtcagggaactaagatc

SEQ ID NO: 854 - DNA, 119 bp, Bos taurus
  misc_feature (60)..(60): A OR G
cagggacttccctggcagtccaattgttaagactgtgcacatctactgtaggggtcatgngtttgatccctagtcagggaactaagatcccatatgccactcagcatggccaaaaaaat

SEQ ID NO: 855 - DNA, 119 bp, Bos taurus
  misc_feature (60)..(60): T OR C
tccctggcagtccaattgttaagactgtgcacatctactgtaggggtcatgggtttgatncctagtcagggaactaagatcccatatgccactcagcatggccaaaaaaataaaacagt

SEQ ID NO: 856 - DNA, 119 bp, Bos taurus
  misc_feature (60)..(60): C OR T
tgtgcacatctactgtaggggtcatgggtttgatccctagtcagggaactaagatcccanatgccactcagcatggccaaaaaaataaaacagtacttctcatgattctctagggtggc

SEQ ID NO: 857 - DNA, 119 bp, Bos taurus
  misc_feature (60)..(60): C OR G
tcccatatgccactcagcatggccaaaaaaataaaacagtacttctcatgattctctagngtggcaacccttagctggaggattctttagtttcaggaggtcagcagggtagagggtct

SEQ ID NO: 858 - DNA, 119 bp, Bos taurus
  misc_feature (60)..(60): C OR CA
tatgccactcagcatggccaaaaaaataaaacagtacttctcatgattctctagggtggnaacccttagctggaggattctttagtttcaggaggtcagcagggtagagggtctggatt

SEQ ID NO: 859 - DNA, 119 bp, Bos taurus
  misc_feature (60)..(60): C OR G
tctctagggtggcaacccttagctggaggattctttagtttcaggaggtcagcagggtanagggtctggatttttgacacttaagtttctggtctgaaaatgtcaggtttccaactgca

SEQ ID NO: 860 - DNA, 119 bp, Bos taurus
  misc_feature (60)..(60): G OR A
ggatttttgacacttaagtttctggtctgaaaatgtcaggtttccaactgcaaaagtacnaacagctaaaatatcacttctggcacctttgattagtgagaagtccagcccacaccctt

SEQ ID NO: 861 - DNA, 119 bp, Bos taurus
  misc_feature (60)..(60): C OR T
cacccttaaggaaaattagctcttattggaaggagcagcactcatatacagagatgggantgttggtgaccatttctggaggtgacttaccacaccttttttttgagataagggtatag

SEQ ID NO: 862 - DNA, 119 bp, Bos taurus
  misc_feature (60)..(60): C OR T
agatttctaaaaactctgcatatgattgaataaaacgtcagtggtttccaaaacatgtancgagttaagaaacactggttttgtgaatggacatgactggtccagggtaaagatgttct

SEQ ID NO: 863 - DNA, 119 bp, Bos taurus
  misc_feature (60)..(60): A OR G
ggtccagggtaaagatgttctcacacagagcatccagggggagcagcggagccaaagctntacgacaacacaggttattttctccactcgggcacaaatcaacaaaccccaaaccccag

SEQ ID NO: 864 - DNA, 119 bp, Bos taurus
  misc_feature (60)..(60): G OR A
tccagggtaaagatgttctcacacagagcatccagggggagcagcggagccaaagctgtncgacaacacaggttattttctccactcgggcacaaatcaacaaaccccaaaccccagaa

SEQ ID NO: 865 - DNA, 119 bp, Bos taurus
  misc_feature (60)..(60): A OR C
ggacctcacttaatgggacctgtcacctgggcctgcacaaaccatgcacaagccgagggntgagtgaaggctggcaggtggggctcccgtctaccttttcactggagctttctcaaacc

SEQ ID NO: 866 - DNA, 119 bp, Bos taurus
  misc_feature (60)..(60): C OR CA
aactgctcttcaaaggagaaaaaggcagacatgtttatgcctagtgactaatatacttcnaaaaaaactgcttttcactcatttcatattccagaaggaatgcatgatggaagcaagtt

SEQ ID NO: 867 - DNA, 119 bp, Bos taurus
  misc_feature (60)..(60): T OR C
gaatcatcttgccatataaagggcacaccatattcttaaaaactacttaatgtgactcantgttccccctagtgagaaacaggaaagcaggcaccgaggcatcagatgactcacagtag

SEQ ID NO: 868 - DNA, 119 bp, Bos taurus
  misc_feature (60)..(60): G OR A
ggcacaccatattcttaaaaactacttaatgtgactcactgttccccctagtgagaaacnggaaagcaggcaccgaggcatcagatgactcacagtagttttgtcagaagcatgggatc

SEQ ID NO: 869 - DNA, 119 bp, Bos taurus
  misc_feature (60)..(60): C OR T
ttccccctagtgagaaacaggaaagcaggcaccgaggcatcagatgactcacagtagttntgtcagaagcatgggatctggactcaagagaagcaacgctttcattctttttctataac

SEQ ID NO: 870 - DNA, 119 bp, Bos taurus
  misc_feature (60)..(60): C OR CCTG
agaaccagaatgaggcacatctctgtatcctggtttaggtctctgacctacaaatgctgnctgctgctgctgctgctaagtcgcttcagtcgtgtccgactctgcgtgaccccatagtc

SEQ ID NO: 871 - DNA, 119 bp, Bos taurus
  misc_feature (60)..(60): C OR T
ctacaaatgctgcctgctgctgctgctgctaagtcgcttcagtcgtgtccgactctgcgngaccccatagtcggcagcccactaggcacccctgtccctgggattctctaggcaagaac

SEQ ID NO: 872 - DNA, 119 bp, Bos taurus
  misc_feature (60)..(60): T OR TGGGATTCTCTA
cagtcgtgtccgactctgcgtgaccccatagtcggcagcccactaggcacccctgtcccngggattctctaggcaagaacattggagtgggttgccatttccttctccaatgcatgaaa

SEQ ID NO: 873 - DNA, 119 bp, Bos taurus
  misc_feature (60)..(60): G OR A
catttccttctccaatgcatgaaagtgaaaagggaaagtgaagtcgctcagttgtacccnactcttagcgaccccatggactgcagcctaccaggctcctccgtccatgggattttcca

SEQ ID NO: 874 - DNA, 119 bp, Bos taurus
  misc_feature (60)..(60): A OR T
ccccatggactgcagcctaccaggctcctccgtccatgggattttccaggcaagagtatnggagtgggtcgccactgccttctccaaatgctgcctacaggagtaactaaactcaattc

SEQ ID NO: 875 - DNA, 119 bp, Bos taurus
  misc_feature (60)..(60): C OR T
gaagcaatcagcacatgctgaggcttttttcctctagaaatggttggtgcttccacttanggagtctcagagatgcaggaatgactcgagacttttgctcttctaacgaaggagcctaa

SEQ ID NO: 876 - DNA, 119 bp, Bos taurus
  misc_feature (60)..(60): A OR G
ttcctctagaaatggttggtgcttccacttatggagtctcagagatgcaggaatgactcnagacttttgctcttctaacgaaggagcctaagaagctaatcttcttttgtacaggtaca

SEQ ID NO: 877 - DNA, 119 bp, Bos taurus
  misc_feature (60)..(60): A OR G
tttaaagcagagaaatcttcctttgatttctaggtgaggaaagagggatgacaatcaccnaagacttgtgatctgcactgtctatccactatcattgaggtccacattaccttcctcaa

SEQ ID NO: 878 - DNA, 119 bp, Bos taurus
  misc_feature (60)..(60): T OR C
ggatgacaatcaccgaagacttgtgatctgcactgtctatccactatcattgaggtccanattaccttcctcaatttttccaaaatgggagagacaagagaatttccctttgatgcaaa

SEQ ID NO: 879 - DNA, 119 bp, Bos taurus
  misc_feature (60)..(60): A OR G
tgttcccacttcccacagctatcccacctagccttacctcctgctgggtcttcaaatggntaacagaaactactcacagctacgcatacctgttagctggaaccagagtgtgacacaca

SEQ ID NO: 880 - DNA, 119 bp, Bos taurus
  misc_feature (60)..(60): G OR A
aaatgggtaacagaaactactcacagctacgcatacctgttagctggaaccagagtgtgncacacagaatttactccagcaactagacaagctcagagttaagcactcccgccagatag

SEQ ID NO: 881 - DNA, 119 bp, Bos taurus
  misc_feature (60)..(60): T OR G
agagtgtgacacacagaatttactccagcaactagacaagctcagagttaagcactcccnccagatagcctgcatcagaggaaaaaacaactttccccacccacaaatcatgtccctga

SEQ ID NO: 882 - DNA, 119 bp, Bos taurus
  misc_feature (60)..(60): A OR G
aaacctcaagtgcatttattagtggtaggccagcggcaccatttcacactcaaatgaganttcctgggtgaaacaaatttcagcagagaacatgctttattgaagagtagatatagaac

SEQ ID NO: 883 - DNA, 119 bp, Bos taurus
  misc_feature (60)..(60): C OR T
caaaagctgccccaagatagcaggttataatgggctagagaaattagagggagcatctcntccttcacttaaacgacaccaaaaatagaggagaccagagaatggggggtggtggtaaa

SEQ ID NO: 884 - DNA, 119 bp, Bos taurus
  misc_feature (60)..(60): G OR A
gaaattagagggagcatctcttccttcacttaaacgacaccaaaaatagaggagaccagngaatggggggtggtggtaaatcccaaaaaggaaatggagttcttggaagggcataagca

SEQ ID NO: 885 - DNA, 119 bp, Bos taurus
  misc_feature (60)..(60): C OR G
tttcattccctttcaccaagatgttgcccactgttcacaggtgaactcaaatccaaatanagaatcaccacctctcaattcttttctccatctcatttagagcatatgctgtaaccaag

SEQ ID NO: 886 - DNA, 119 bp, Bos taurus
  misc_feature (60)..(60): G OR A
gtaaccaagactaatccctgcaagagtcttcagaagtatttggctctggtcagcctgatnctgattaaaaacaggtctttcagtaaaaatggaggaaataacttaaagtgagatgaaat

SEQ ID NO: 887 - DNA, 119 bp, Bos taurus
  misc_feature (60)..(60): A OR T
catggcccacccttgggcctgtggctctgggcagtggccagtgcgtgaccatagtatttnccccacagcatgctgaatgcagcttaggagggaaacaggccagagctgcaaatagggtg

SEQ ID NO: 888 - DNA, 119 bp, Bos taurus
  misc_feature (60)..(60): T OR G
actaccctgcaagctttgcgtggttaataccatagctactctagtcaatatctctagatntttttctcaagttttgcttcttgggagatttatcagatttgtcgagattctaccaatga

SEQ ID NO: 889 - DNA, 119 bp, Bos taurus
  misc_feature (60)..(60): T OR A
atttcatctgcgtagaatcctacagtttatactcttttgtgtctggtttctttggatatntatttgaattttcagattttctattttgtgcacgtgtcacttggtaatttgtgtctttc

SEQ ID NO: 890 - DNA, 119 bp, Bos taurus
  misc_feature (60)..(60): T OR C
ggcagctgcctgtggggctggatgcataggtggccccagcatttgctgtgctcctctggntccgaggccacatatttcctggggttcctcaccctcgtgtctgctcgctgcttcccagc

SEQ ID NO: 891 - DNA, 119 bp, Bos taurus
  misc_feature (60)..(60): T OR G
gtgctcctctggctccgaggccacatatttcctggggttcctcaccctcgtgtctgctcnctgcttcccagcagggtagggagctggtctggaccttgaaagtggcactgctcccttct

SEQ ID NO: 892 - DNA, 119 bp, Bos taurus
  misc_feature (60)..(60): G OR A
ctgagtgatcaccccacagagaccaaggcagcaccgccaggcaggctgctagggcaaacncagccacagccacagggacagaagattcaccttgggagtgggctacactgcaactttga

SEQ ID NO: 893 - DNA, 119 bp, Bos taurus
  misc_feature (60)..(60): G OR C
atcttttacttttttttccttgtgttatttcccctgactgaactctttattatgatgttnatcatagctgactttcttcctgatcccggagaagaattttcaacactttacttttaagt

SEQ ID NO: 894 - DNA, 119 bp, Bos taurus
  misc_feature (60)..(60): T OR C
gtgttatttcccctgactgaactctttattatgatgttgatcatagctgactttcttccngatcccggagaagaattttcaacactttacttttaagtttaattgttacttttttgtat

SEQ ID NO: 895 - DNA, 119 bp, Bos taurus
  misc_feature (60)..(60): T OR C
tattccatttgttttgagactgtattatattgcctgttggaattgctttaatctgtcttnttgataggtcttttatcatccaaggtacaatttttcttgttgttggtataacgagtgat

SEQ ID NO: 896 - DNA, 119 bp, Bos taurus
  misc_feature (60)..(60): C OR T
aaaacaatcttctgtggctataaatacttttgaaaggacagctttagatgcactccttangttctgataagccatgcttttatggtaattcggctcaaagtaatttctaatagccactg

SEQ ID NO: 897 - DNA, 119 bp, Bos taurus
  misc_feature (60)..(60): T OR C
tgagccaaggcctttctctgatgttccctttgccatctcacctccatatgcccccttcangccacatgccaggcatgtgctggaggcactgctgtgcggtcagcgtggctccaaggcca

SEQ ID NO: 898 - DNA, 119 bp, Bos taurus
  misc_feature (60)..(60): A OR G
acctgcttccctcggggccttggacaccactggctcttcctgggcccctgcccggcacanaggcagccacacacattctcctttttccctttctcttttccccacaccccaccccgttc

SEQ ID NO: 899 - DNA, 119 bp, Bos taurus
  misc_feature (60)..(60): C OR T
gtaccgcctgggcaagggggctcagagcctgggtgtttttcccgggactacctcctggcncacaagctgagaccccaagcagccactgctcctgcctttggcctcctttgatttctgca

SEQ ID NO: 900 - DNA, 119 bp, Bos taurus
  misc_feature (60)..(60): TC OR T
gctcctggagctgggaaaccggctgcgagcggagctggggcaccttaccggtgggtcatnccccccactccgcccctgctctggggatggaggtgcagggtgcagggcagggctggccc

SEQ ID NO: 901 - DNA, 119 bp, Bos taurus
  misc_feature (60)..(60): T OR C
agtgtgcgagcagcaaccgagggcgtctttcacgggagctggctccttcccttgggaccngtttgacaaagccctgctttcgttcagtcctccttctgttccacaagttgaaggggaag

SEQ ID NO: 902 - DNA, 119 bp, Bos taurus
  misc_feature (60)..(60): G OR A
gtgtgcgagcagcaaccgagggcgtctttcacgggagctggctccttcccttgggacctntttgacaaagccctgctttcgttcagtcctccttctgttccacaagttgaaggggaagg

SEQ ID NO: 903 - DNA, 119 bp, Bos taurus
  misc_feature (60)..(60): C OR CT
gtcatagagtggggatcacacaatatgtattctctgatacctaacatttcttaaaggctntttttttttttcccttgatgttgaaaatgctatttgctcactataaaaatttcaatgaa

SEQ ID NO: 904 - DNA, 119 bp, Bos taurus
  misc_feature (60)..(60): G OR GT
agaggcagtaccctgacagctcctcaggatgtgcggtaggcaatgcagtttccatagctntttttttttttccctttagtttcttagcttttgatacaattcatgtgaaagattattct

SEQ ID NO: 905 - DNA, 119 bp, Bos taurus
  misc_feature (60)..(60): T OR A
ccttttttcttgaggtttccagaatatcctgaggctcctggacctgccaggagatgaacnttcttatcccctcatgaggctggcacctggcagttttcctgggagggccttatgagcct

SEQ ID NO: 906 - DNA, 119 bp, Bos taurus
  misc_feature (60)..(60): A OR G
aagttgggaggagctgttaaaaacagagtccattggaacaaattggaacttcaaagcacngtggcttttattggctgggccattgccaggcagagataaaatccttctccagctgccaa

SEQ ID NO: 907 - DNA, 119 bp, Bos taurus
  misc_feature (60)..(60): C OR G
aatgggtcagggggacagagaagcctagggaggcagtgtgccagaccaggagacactggnctgagaaacagaaaggctggctctgagaggggcccaggaggcacagtgttttccacaac

SEQ ID NO: 908 - DNA, 119 bp, Bos taurus
  misc_feature (60)..(60): A OR G
ctgttttcagacgtgggaaaataggtagcagaggaccgggatccttgaacgaggaagacnaacgaggcgcctgtcgggccccctggctttctgccccgaggcatcttctgggctcagtc

SEQ ID NO: 909 - DNA, 119 bp, Bos taurus
  misc_feature (60)..(60): C OR T
gccaggcccctctgtccatgagattctccaggcaagaatactggagtgggttgccatttnctcctccacgggatctttctgaccgacccagggatcgaaccgaggtctcctgcgttaca

SEQ ID NO: 910 - DNA, 119 bp, Bos taurus
  misc_feature (60)..(60): T OR C
ttcttgttcccaagttcgttctgttcctttctctccagtttctccccctgaaggctagtncatctcctggtagatgagatgttagtatttccagataagtgtattggcagccgcttgag

SEQ ID NO: 911 - DNA, 119 bp, Bos taurus
  misc_feature (60)..(60): G OR C
gagcacccagcaccgacagcctgtagcctccagcaccctgaggggagccccactgtgacntgggcagcctttggcgttgaagggatgaagatggaagcccagacaaggggcaagcctac

SEQ ID NO: 912 - DNA, 119 bp, Bos taurus
  misc_feature (60)..(60): C OR G
tttctgacctcccaagggtctatcagtggtcccaggagatagtaagggccgtgcagggcntggccaccagtcagtccttcctgggctcacgtgattcccgatccgtgttgttttcagtc

SEQ ID NO: 913 - DNA, 119 bp, Bos taurus
  misc_feature (60)..(60): A OR G
ggccttggcactgttgaggagaccaagctgtggttctggcctggagggcagctcgagatnctgagcgcagagccagctagtcagtgtgggcacttcttcccacccccacatttgccctt

SEQ ID NO: 914 - DNA, 119 bp, Bos taurus
  misc_feature (60)..(60): G OR A
tgcacaccagaggaatcccagacatgtcctgtcaccccagtttcctcactccccattgcntggctgatcttggaggcgagggcaggacaccaggtgccaggcagagcaggaggcaggat

SEQ ID NO: 915 - DNA, 119 bp, Bos taurus
  misc_feature (60)..(60): G OR A
gacccttgagtgtgacagggtcctggacaggcactgcctggggggaggggcggggaggcngaggccttcccactgcccaggccccagccggagaccttggatgctgctctccaggggct

SEQ ID NO: 916 - DNA, 119 bp, Bos taurus
  misc_feature (60)..(60): T OR C
agacaaagagatggctggatggcatcactgactagatggacgtgagtctgagtgaactcngggagttggtgatggacagggaggcctggcgtgctgtgattcatggggtcgcaaagagt